(19) United States
(12) Patent Application Publication
(10) Pub. No.: US 2024/0398926 A1
(43) Pub. Date: Dec. 5, 2024

(54) NOVEL ADENOVIRUS VACCINE THERAPY FOR THE TREATMENT OF RECURRENT RESPIRATORY PAPILLOMATOSIS

(71) Applicant: PRECIGEN, INC., Germantown, MD (US)

(72) Inventors: Douglas E. BROUGH, Germantown, MD (US); Damodar R. ETTYREDDY, Germantown, MD (US); Qi YANG, Germantown, MD (US); Chen WANG, Germantown, MD (US)

(73) Assignee: PRECIGEN, INC., (US)

(21) Appl. No.: 18/671,685

(22) Filed: May 22, 2024

Related U.S. Application Data
(60) Provisional application No. 63/468,112, filed on May 22, 2023.

(51) Int. Cl. A61K 39/12; A61K 39/00; A61P 31/20; C07K 14/005; C12N 7/00; C12N 15/86

(52) U.S. Cl. CPC A61K 39/12; A61P 31/20; C07K 14/005; C12N 7/00; C12N 15/86; A61K 2039/575; C12N 2710/10043; C12N 2710/20022; C12N 2710/20034

(57) ABSTRACT

Multi-antigenic human papilloma virus (HPV) molecular vaccine constructs for use and treatment of HPV-associated disorders and pathologies, such as HPV molecular vaccines targeting HPV6- and HPV11-associated recurrent respiratory papillomatosis (RRP).

### CROSS REFERENCE TO RELATED APPLICATIONS

This application claims the benefit of priority of U.S. Provisional Application No. 63/468,112, filed May 22, 2023, which is incorporated herein by, reference in its entirety.

### SEQUENCE LISTING

The instant application contains a Sequence Listing which has been submitted electronically in XML file format and is hereby incorporated by reference in its entirety. Said XML copy, created on May 17, 2024, is named 75594-408688_SL.xml and is 271,361 bytes in size.

### BACKGROUND OF THE INVENTION

Recurrent respiratory papillomatosis (RRP) is a rare, difficult-to-treat, and sometimes fatal neoplastic disease of the upper and lower respiratory tracts. RRP is caused by infection with human papillomavirus (HPV) type 6 or 11. Mounts P, Shah K V, Kashima H: Viral etiology of juvenile- and adult-onset squamous papilloma of the larynx. Proc Nat Acad Sci USA 1982, 79(17):5425-5429. Approximately 1,500 new cases of RRP are diagnosed each year in the United States. Derkay C S, Wiatrak B: Recurrent respiratory papillomatosis: a review. Laryngoscope 2008, 118(7):1236-1247.

RRP is classified based on age of onset as juvenile or adult. Juvenile-onset disease has an incidence of 4/100,000 and tends to have an aggressive clinical course. Adult-onset RRP has an incidence of 2-3/100,000 and tends to have a more indolent clinical course.

RRP morbidity and mortality results from papilloma mass effects on the vocal cords, trachea, and lungs. This may cause voice changes, stridor, airway occlusion, loss of lung volume, and/or post-obstructive pneumonia. Silver R D, Rimell F L, Adams G L, Derkay C S, Hester R: Diagnosis and management of pulmonary metastasis from recurrent respiratory papillomatosis. Otolaryngol Head Neck Surg 2003, 129(6):622-629. Repeated procedures are required to debulk and monitor the disease, which exposes participants to anesthetic and surgical risk, and emotional distress. It is estimated that the economic cost of RRP is $150M in the United States each year. Derkay C S, Wiatrak B: Recurrent respiratory papillomatosis: a review. Laryngoscope 2008, 118(7):1236-1247. Although rare (one to three percent of cases) RRP can transform into invasive squamous cell carcinoma. Dedo H H, Yu K C: CO(2) laser treatment in 244 patients with respiratory papillomas. Laryngoscope 2001, 111(9):1639-1644. Subsequent mortality is based upon the clinical stage of the malignancy at the time of diagnosis.

There is no cure for RRP, no approved medical therapies currently exist, and complete regression of RRP has not yet been observed using immunotherapy. The mainstay of treatment for RRP is repeated endoscopic debulking with ablation or excision of papillomatous lesions. Surgical principles dictate that, to minimize morbidity from treatment, papillomatous disease but not normal appearing epithelium is removed. It is thought that latent HPV viral particles persist in an inactive state in the clinically-normal mucosa and subsequently become reactivated leading to RRP recurrence. Armstrong L R, Derkay C S, Reeves W C: Initial results from the national registry for juvenile-onset recurrent respiratory papillomatosis. RRP Task Force. Arch Otolaryngol Head Neck Surg 1999, 125(7):743-748.

Participants with juvenile-onset RRP require on average 20 surgeries over their lifetime to control their disease. Id. Participants with adult-onset RRP generally require fewer interventions; nonetheless greater than 50% will require 5 or more procedures to control symptoms. Kashima H K, Shah F, Lyles A, Glackin R, Muhammad N, Turner L, Van Zandt S, Whitt S, Shah K: A comparison of risk factors in juvenile-onset and adult-onset recurrent respiratory papillomatosis. Laryngoscope 1992, 102(1):9-13. Some individuals with more aggressive disease may require hundreds of lifetime surgeries to maintain a useable voice and patent airway. Adjuvant systemic therapies have been tested in clinical trials, including systemic interferon-α and local injection of anti-viral and antiangiogenic agents. Study results have been inconsistent, and no single adjuvant approach has been widely adopted or accepted as the standard of care.

Local immunotherapies fail to eradicate RRP apparently due to chronic persistence of latent HPV in normal appearing mucosa. This notion is supported by a study demonstrating the presence of HPV DNA in the clinically healthy mucosa of participants with RRP. Smith E M, Pignatari S S, Gray S D, Haugen T H, Turek L P: Human papillomavirus infection in papillomas and nondiseased respiratory sites of patients with recurrent respiratory papillomatosis using the polymerase chain reaction. Arch Otolaryngol Head Neck Surg 1993, 119(5):554-557. Previous efforts to study systemic immunotherapy for RRP have been limited. Adjuvant IFN-α after papilloma treatment was shown to increase short-term time to recurrence but did not demonstrate long-term benefit.

Programmed death ligand 1 (PD-L1) expression on tumor cells has been associated strongly with poor prognosis in a variety of human cancers. Healy G B, Gelber R D, Trowbridge A L, Grundfast K M, Ruben R J, Price K N: Treatment of recurrent respiratory papillomatosis with human leukocyte interferon. Results of a multicenter randomized clinical trial. N Engl J Med 1988, 319(7):401-407. In recent years, a number of agents targeting the programmed death 1 (PD-1)/PD-L1 pathway have received regulatory approval, demonstrating impressive durations of response for multiple tumor types, including head and neck cancer. Seiwert T Y, Burtness B, Mehra R, Weiss J, Berger R, Eder J P, Heath K, McClanahan T, Lunceford J, Gause C et al: Safety and clinical activity of pembrolizumab for treatment of recurrent or metastatic squamous cell carcinoma of the head and neck (KEYNOTE-012): an open-label, multicentre, phase 1b trial. Lancet Oncol 2016, 17(7):956-965; Ferris R L, Blumenschein G, Jr., Fayette J, Guigay J, Colevas A D, Licitra L, Harrington K, Kasper S, Vokes E E, Even C et al: Nivolumab for Recurrent Squamous-Cell Carcinoma of the Head and Neck. N Engl J Med 2016, 375(19):1856-1867. Notably, atezolizumab, durvalumab and avelumab are all anti-PD-L1 antibodies with proven efficacy and regulatory approval. None of these anti-PD-L1 antibodies, however, have been shown to safely and effectively cure adults with RRP.

Thus, there remains in the art a need for relatively non-invasive methods to safely and effectively treat adults with RRP, for both cost and emotional benefits to the patient, including methods that focus on the priming of T-cell responses against HPV antigens.

Therapeutic vaccines are one immunotherapy strategy that may enhance de novo T-cell responses from individuals with RRP. Therapeutic vaccination of participants with a long peptide vaccine encoding HPV-16 antigens induced complete regression of disease in nearly 50% of participants with pre-malignant vulvar intraepithelial neoplasia. Kenter G G, Welters M J, Valentijn A R, Lowik M J, Berends-van der Meer D M, Vloon A P, Essahsah F, Fathers L M, Offringa R, Drijfhout J W et al: Vaccination against HPV-16 oncoproteins for vulvar intraepithelial neoplasia. N Engl J Med 2009, 361(19):1838-1847. The magnitude of HPV 16-specific T-cell responses correlated with the magnitude of clinical response in these participants. van Poelgeest M I, Welters M J, Vermeij R, Stynenbosch L F, Loof N M, Berends-van der Meer D M, Lowik M J, Hamming I L, van Esch E M, Hellebrekers B W et al: Vaccination against Oncoproteins of HPV16 for Noninvasive Vulvar/Vaginal Lesions: Lesion Clearance Is Related to the Strength of the T-Cell Response. Clin Cancer Res 2016, 22(10):2342-2350. Durable remission without recurrence of papillomatous lesions in children and adults with RRP has been reported from Mexico City following high frequency intralesional injections (4 injections total, once every two weeks, injected directly into papillomas) of a modified vaccinia Ankara virus encoding bovine papilloma virus E2. Cabo Beltran OR, Rosales Ledezma R: MVA E2 therapeutic vaccine for marked reduction in likelihood of recurrence of respiratory papillomatosis. Head Neck 2019, 41(3):657-665. Though this study demonstrated clinical activity, additional improvements are needed, and translational research defining the mechanism of papilloma clearance is lacking.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Provided herein are non-naturally occurring polynucleotides (and polypeptides expressed therefrom) encoding non-naturally occurring polypeptides comprising immune response-inducing human papilloma virus (HPV) polypeptides. Also provided herein are non-naturally occurring, variably arrayed, configurations of HPV immune response-inducing polypeptides connected by various polypeptide linker sequences; thereby, comprising fusion proteins useful as vaccine antigens.

The invention includes, but is not limited to, a composition (e.g., a substance comprising a polynucleotide, polypeptide, vector, vaccine, or cell), methods of making and delivering a composition, and uses of non-naturally-occurring polynucleotides encoding non-naturally occurring polypeptides comprising antigens for vaccines directed against human papillomavirus (HPV), in particular HPV6 and HPV11; and, therapeutic approaches to treat pathological conditions caused by these particular HPV agents.

In particular, the invention comprises polynucleotides, and polypeptides encoded by same, encoding multi-antigenic polypeptides derived from HPV6, HPV11 and HPV16 and other polypeptide sequences for use as vaccine components and for treatment of disorders associated with HPV infection.

For example, an embodiment of the present invention relates to the use of compositions described herein as therapeutic vaccines against HPV6 and/or HPV11 (HPV6/11) induced or associated diseases; such as, but not limited to, treatment of recurrent respiratory papillomatosis (RRP), anogenital warts, and other HPV6/11-associated diseases, such as lower genital tract neoplasia (e.g., cervical, vaginal, and vulvar intraepithelial neoplasia), cervical cancer, vulvar cancer, anal cancer, penile cancer, and head and neck cancers. In one embodiment, the compositions described herein are used as therapeutic vaccines against RRP.

The present invention includes unique and innovative HPV polypeptide vaccine design approaches for HPV6/11 induced diseases. The invention includes a mix of design strategies; such as use of full protein sequences, use of peptide “fragments,” hybrid polypeptide constructs, introduction of amino acid substitutions, insertions, deletions and rearrangement of polypeptide and peptide HPV gene products (proteins).

As indicated above, unique modifications of antigens described herein have been made by introducing point mutations, substitution mutations, and/or reordering of viral polypeptide sequences, in particular, to prevent oncogenic gene expression and/or disable essential viral functions (e.g. viral replication).

In certain embodiments, HPV early proteins E2 and E4 have been identified as novel antigens for HPV6/11 and incorporated into vaccine designs described herein.

In certain embodiments, the invention comprises the novel incorporation of four antigenic components from HPVs into one HPV vaccine design.

In certain embodiments, the invention comprises the novel incorporation of four antigenic components from HPVs into one HPV vaccine design.

In certain embodiments, the invention comprises the novel integration and combination of both high cancer risk and low cancer risk HPV genotype epitopes into one HPV antigen construct.

In certain embodiments, the HPV antigen construct comprises a polynucleotide encoding a polypeptide having at least 90% sequence identity with SEQ ID NO: 68 or a functional variant thereof (e.g., a polypeptide having at least 80%, 85%, 90%, 95%, 96%, 97%, 98%, or 99% sequence identity with SEQ ID NO: 68 or a conservatively-substituted variant of SEQ ID NO: 68).

In certain embodiments, the vector is an adenoviral vector, for example, a gorilla adenoviral vector. In some such embodiments, the vector is derived from GC44, GC45, or GC46.

In certain embodiments, the vector is an adenoviral vector lacking all or a portion of the E1 and/or E4 regions of GC46.

In certain embodiments, the vector comprises a nucleic acid sequence having at least 90% sequence identity with SEQ ID NO: 119 or a functional variant thereof (e.g., a nucleic acid having at least 80%, 85%, 90%, 95%, 96%, 97%, 98%, or 99% sequence identity with SEQ ID NO: 119 or a codon degenerate variant of SEQ ID NO: 119).

In certain embodiments, the expression cassette comprises a promoter and expression of the transgene is under the control of the promoter. In some such embodiments, the promoter is a cytomegalovirus promoter or a synthetic promoter.

In certain embodiments, the promoter is a synthetic promoter. In some such embodiments, the synthetic promoter comprises a blocking sequence, an enhancer, and/or a responsive element. In certain embodiments, the enhancer comprises a nucleic acid sequence having at least 90% sequence identity with SEQ ID NO: 96 or a functional variant thereof (e.g., a nucleic acid having at least 80%, 85%, 90%, 95%, 96%, 97%, 98%, or 99% sequence identity with SEQ ID NO: 96 or a codon degenerate variant of SEQ ID NO: 96). In certain embodiments, the responsive element comprises a nucleic acid sequence having at least 90% sequence identity with SEQ ID NO: 98 or a functional variant thereof (e.g., a nucleic acid having at least 80%, 85%, 90%, 95%, 96%, 97%, 98%, or 99% sequence identity with SEQ ID NO: 98 or a codon degenerate variant of SEQ ID NO: 98).

In certain embodiments, the expression cassette comprises a 5′ UTR. In some such embodiments, the 5′ UTR comprises a nucleic acid sequence having at least 90% sequence identity with SEQ ID NO: 99 or a functional variant thereof (e.g., a nucleic acid having at least 80%, 85%, 90%, 95%, 96%, 97%, 98%, or 99% sequence identity with SEQ ID NO: 99 or a codon degenerate variant of SEQ ID NO: 99).

In certain embodiments, the expression cassette comprises a termination sequence. In some such embodiments, the termination sequence comprises a nucleic acid sequence having at least 90% sequence identity with SEQ ID NO: 104 or a functional variant thereof (e.g., a nucleic acid having at least 80%, 85%, 90%, 95%, 96%, 97%, 98%, or 99% sequence identity with SEQ ID NO: 104 or a codon degenerate variant of SEQ ID NO: 104).

In certain embodiments, the vector is an adenoviral vector and the expression cassette is located in the E1 region deletion junction.

In certain embodiments, the expression cassette is cloned in the right-to-left orientation with respect to the adenovirus viral genome.

In certain embodiments, the vector is an adenoviral vector, for example, a gorilla adenoviral vector. In some such embodiments, the vector is derived from GC44, GC45, or GC46.

In certain embodiments, the vector is an adenoviral vector lacking all or a portion of the E1 and/or E4 regions of GC46.

The present invention also relates in part to a pharmaceutical composition comprising the vector described herein and a pharmaceutically-acceptable carrier.

In certain embodiments, the composition further comprises an additional therapeutic agent. In some such embodiments, the additional therapeutic agent treats pathological conditions caused by HPV agents.

The present invention also relates in part to a kit comprising the pharmaceutical composition described herein. In certain embodiments, the kit further comprises a label.

The present invention also relates in part to a method of treating a disease or disorder, including RRP, in a subject in need thereof, the method comprising administering to the subject the vector described herein.

In certain embodiments of the method, the vector is administered in an amount of about 0.1×10¹¹ to about 1×10¹² viral particle units.

In certain embodiments, the method comprises administering a composition comprising the vector. In some such embodiments, the composition is administered subcutaneously, intramuscularly, intravenously, intracranially, intraarticularly, intradermally, transdermally, intratumorally, or intralesionally. In some embodiments, the composition is administered to the limbs, buttocks, and/or abdomen. In some embodiments, the composition is administered to the lungs or upper airways via an aerosol spray or mist.

In certain embodiments, the method comprises administering a composition comprising the vector in doses. In some embodiments, each dose may comprise about 0.1×10¹¹ to about 5×10¹¹ viral particle units. In some embodiments, each dose is administered at least 11 days apart. In certain embodiments, the second dose is administered two weeks after the first dose, the third dose is administered six weeks after the second dose, and the fourth dose is administered twelve weeks after the third dose. In some embodiments, at least one dose is administered at least a month apart from the previous dose.

In some embodiments, a debulking procedure is performed before the first dose is administered. In some embodiments, one or more debulking procedures is performed 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months before the first dose is administered. In one embodiment, three debulking procedures are performed before the first dose is administered. In another embodiment, one or more debulking procedures are performed 12 months before the first dose is administered. In a further embodiment, three debulking procedures are performed 12 months before the first dose is administered.

In some embodiments, one or more debulking procedures is performed after the first dose is administered and before the final dose is administered. In some embodiments, one or more debulking procedures is performed after the final dose is administered. In some embodiments, one or more debulking procedures is performed during the administration of at least one dose of the composition.

In one embodiment, a patient may receive at least one debulking procedure at least 6 months, 1 year, or 2 years following administration of a dose of the composition described herein, or in a time frame determined by the patient's physician, where the patient may then, subsequent to such a debulking procedure, proceed to receive one or more additional doses of the composition. In some embodiments, clinical efficacy of the therapeutic method is measured after the final dose is administered.

In some embodiments, administration of the vector described herein results in a reduced need for surgical intervention (e.g., debulking surgery).

In some embodiments, administration of the vector described herein results in an increase in HPV6/11 antigen-specific T-cell responses.

In some embodiments, administration of the vector described herein results in an improved Derkay score.

In certain embodiments, the method further comprises the administration of one or more additional therapeutic agents. In some such embodiments, the additional therapeutic agent(s) may be a chemotherapy agent, an anti-inflammatory agent, an analgesic, and/or a biological response modifier.

Also provided herein are polynucleotides encoding a fusion protein comprising (a) an HPV6 protein and (b) an HPV11 protein. In some embodiments, the polynucleotide described herein encodes a fusion protein comprising (a) an HPV6 protein selected from an HPV6 E2 protein, an HPV6 E4 protein, an HPV6 E6 protein, and an HPV6 E7 protein; and (b) an HPV11 protein selected from an HPV11 E6 and an HPV11 E7 protein. In some embodiments, the polynucleotide described herein comprises an HPV6 E2 protein; an HPV6 E4 protein; an HPV6 E6 protein; an HPV6 E7 protein; an HPV11 E6; and an HPV11 E7 protein. In some embodiments, the HPV6 E2 protein comprises an amino acid sequence having at least 90% sequence identity with SEQ ID NO: 1. In some embodiments, the HPV6 E2 protein comprises the amino acid sequence of SEQ ID NO: 1. In some embodiments, the HPV6 E4 protein comprises an amino acid sequence having at least 90% sequence identity with SEQ ID NO: 3 or 7. In some embodiments, the HPV6 E4 protein comprises the amino acid sequence of SEQ ID NO: 3 or 7. In some embodiments, the HPV6 E6 protein comprises an amino acid sequence having at least 90% sequence identity with SEQ ID NO: 11 or 40. In some embodiments, the HPV6 E6 protein comprises the amino acid sequence of SEQ ID NO: 11 or 40. In some embodiments, the HPV6 E7 protein comprises an amino acid sequence having at least 90% sequence identity with SEQ ID NO: 5 or 9. In some embodiments, the HPV6 E7 protein comprises the amino acid sequence of SEQ ID NO: 5 or 9. In some embodiments, the HPV11 E6 protein comprises an amino acid sequence having at least 90% sequence identity with SEQ ID NO: 42. In some embodiments, the HPV11 E6 protein comprises the amino acid sequence of SEQ ID NO: 42. In some embodiments, the HPV11 E7 protein comprises an amino acid sequence having at least 90% sequence identity with SEQ ID NO: 45. In some embodiments, the HPV11 E7 protein comprises the amino acid sequence of SEQ ID NO: 45.

In some embodiments, the fusion protein comprises an HPV6 E4 protein comprising the amino acid sequence of SEQ ID NO: 3 and an HPV6 E4 protein comprising the amino acid sequence of SEQ ID NO: 7. In some embodiments, the fusion protein comprises an HPV6 E6 protein comprising the amino acid sequence of SEQ ID NO: 11 and an HPV6 E6 protein comprising the amino acid sequence of SEQ ID NO: 40. In some embodiments, the fusion protein comprises an HPV6 E7 protein comprising the amino acid sequence of SEQ ID NO: 5 and an HPV6 E7 protein comprising the amino acid sequence of SEQ ID NO: 9.

In some embodiments, the fusion protein comprises an amino acid sequence having at least 80% identity with SEQ ID NO: 68. In some embodiments, the fusion protein comprises an amino acid sequence having at least 90% identity with SEQ ID NO: 68. In some embodiments, the fusion protein comprises an amino acid sequence having at least 95% identity with SEQ ID NO: 68. In some embodiments, the fusion protein comprises an amino acid sequence having at least 97% identity with SEQ ID NO: 68. In some embodiments, the fusion protein comprises an amino acid sequence having at least 98% identity with SEQ ID NO: 68. In some embodiments, the fusion protein comprises an amino acid sequence having at least 99% identity with SEQ ID NO: 68. In some embodiments, the fusion protein comprises the amino acid sequence of SEQ ID NO: 68 or a conservatively-substituted variant thereof. In some embodiments, the fusion protein comprises the amino acid sequence of SEQ ID NO: 68.

In some embodiments, the fusion protein comprises an amino acid sequence having at least 80% identity with SEQ ID NO: 70. In some embodiments, the fusion protein comprises an amino acid sequence having at least 80% identity with SEQ ID NO: 72. In some embodiments, the fusion protein comprises an amino acid sequence having at least 80% identity with SEQ ID NO: 74.

In some embodiments, the fusion protein further comprises a rigid linker polypeptide. In some embodiments, the fusion protein further comprises an HPV16 E6 agonist enhancer. In some embodiments, the fusion protein further comprises an HPV16 E7 agonist enhancer.

In some embodiments, the fusion protein is operably linked to at least one of: a promoter; a 5′ untranslated region (UTR); a transcription start site (TSS); a 3′ UTR; a tetracycline responsive element; and a kozak region. In some embodiments, the promoter is operably linked to a promoter enhancer region.

Also provided herein are vectors comprising any of the described herein. In some embodiments, the vector is a plasmid, a viral vector, or a non-viral vector. In some embodiments, the viral vector is an adenoviral vector. In some embodiments, the adenoviral vector is deficient in one or more elements selected from an E1-E4 region and an L1-L5 region. In some embodiments, the adenoviral vector comprises one or more elements selected from E2B, L1, L2, L3, E2A, L4, E3, L5, inverted terminal repeat (ITR), poly(a) site, and a spacer. In some embodiments, the adenoviral vector is a gorilla adenoviral vector. In some embodiments, the adenoviral vector is a GC46 gorilla adenoviral vector.

Also provided herein are methods of inducing an anti-HPV immune response in a subject in need thereof. In some embodiments, the method comprises administering a therapeutically effective amount of any of the vectors described herein to the subject. In some embodiments, the therapeutically effective amount comprises about 1×10¹¹ and about 5×10¹¹ particle units (PU).

Also provided herein are methods of treating an HPV-associated disease or disorder in a subject in need thereof. In some embodiments, the method comprises administering to the subject a therapeutically effective amount of any of the vectors described herein to the subject. In some embodiments, the HPV-associated disease or disorder is a HPV6 associated disease or disorder or an HPV11 associated disease or disorder. In some embodiments, the HPV-associated disease or disorder is a HPV-associated cancer. In some embodiments, the HPV-associated disease or disorder is recurrent respiratory papillomatosis (RRP), anogenital warts, lower genital tract neoplasia, cervical cancer, vulvar cancer, anal cancer, penile cancer, or head and neck cancer. In some embodiments, the HPV-associated disease or disorder is RRP. In some embodiments, the therapeutically effective amount comprises about 1×10¹¹ and about 5×10¹¹ particle units (PU). In some embodiments, the method further comprises administering an additional therapy. In some embodiments, the additional therapy comprises the administration of at least one of the following: an angiogenesis inhibitor, such as Bevacizumab (AVASTIN®) and an immune checkpoint inhibitor, such as a PD-1 inhibitor (e.g., Pembrolizumab (KEYTRUDA®), Nivolumab (OPDIVO®), and Cemiplimab (LIBTAYO®)) and/or a PD-L1 inhibitor (e.g., Atezolizumab (TECENTRIQ®), Avelumab (BAVENCIO®), and Durvalumab (IMFINZI®)). In some embodiments, the method further comprises a debulking procedure.

Also provided herein are fusion proteins encoded by any of the polynucleotides described herein.

Also provided herein are compositions comprising any of the polynucleotides described herein. In some embodiments, any of the compositions described herein are for use in treating a disease or disorder in a subject in need thereof.

Also provided herein are uses of any of the polynucleotides described herein in the manufacture of a medicament for use in treating a disease or disorder in a subject in need thereof.

Also provided herein are kits comprising any of the polynucleotides described herein.

Also provided herein are vaccines comprising any of the polynucleotides described herein. In some embodiments, the vaccine is for use in treating a disease or disorder in a subject in need thereof.

The present invention also relates in part to the use of the vector described herein in the manufacture of a medicament for use in treating a disease or disorder, like RRP, in a subject in need thereof.

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

### BRIEF DESCRIPTION OF FIGURES

FIG. 1A depicts a GC46 vector engineered with E1 and E4 region deletions and the CMV HPV6/11 expression cassette in the E1 region (AdV-HPV6/11).

FIG. 1B depicts the HPV-6/11 proteins (HPV-E2, HPV-E4, HPV-E6 and HPV-E7) expressed from a GC46 vector engineer with E1 and E4 region deletions and the CMV HPV6/11 expression cassette in the E1 region (AdV-HPV6/11).

FIG. 1C depicts the protein/antigen expressed from AdV-HPV6/11, which is a fusion of selected regions of HPV proteins that are expressed in HPV-6 and HPV-11 infected cells (HPV-E2, HPV-E4, HPV-E6 and HPV-E7).

FIG. 1D depicts a schematic of a vector backbone containing a CMV promoter, an SV40 polyadenylation signal and 3′ untranslated region, and an antigen open reading frame flanked by inverted terminal repeats (ITRs) and containing the following genes arranged 5′ to 3′: HPV6 E6, HPV11 E7, HPV6 E7, HPV11 E6, HPV6 E4, HPV6 E6, HPV6 E7, HPV11 E6, HPV6 E4, HPV11 E7, and HPV6 E2.

FIG. 2 depicts the fluorescent activity via flow cytometry 24 hours after autologous dendritic cells cultured from participants with recurrent respiratory papillomatosis (RRP) were transduced with base gorilla adenovirus construct encoding GFP (5×10³ MOI).

FIG. 3 depicts IFNg ELISA of T lymphocytes from three RRP participants stimulated with three rounds of dendritic cells transduced with AdV-HPV6/11 or controls. X-axis indicates adenoviral construct tested (differences in construct include gene linker and antigens encoded).

FIG. 4A depicts the experimental design of peripheral T lymphocytes from wild-type C57BL/6 mice vaccinated with AdV-HPV6/11 assessed for HPV antigen-specific immune responses.

FIG. 4B depicts photomicrographs of representative ELISpot wells demonstrating responses to HPV6 and 11 overlapping 15-mer peptide pools as well as synthesized minimal peptides following in vivo vaccination with AdV-HPV6/11 but not empty GC46.

FIG. 4C depicts a quantification of IFNγ spots in vaccinated male (n=3) and female (n=3) mice.

FIG. 4D depicts a quantification of IFNγ spots in an independent validation in vivo vaccination experiment in female wild-type C57BL/6 mice (n=5).

FIG. 4E depicts Quantification (left) and representative flow cytometry dot plots (right) of CD8+ T-lymphocyte IFNγ production following assessment of HPV antigen-specific T-lymphocyte responses from mice vaccinated with AdV-HPV6/11 or empty GC46, measured by intracellular flow cytometry (n=6/group). Dot plots show gated live, CD3+CD8+ T lymphocytes from mice vaccinated with AdV-HPV6/11.

FIG. 5 depicts an ELISpot assessing peripheral T lymphocytes from mice (N=4) treated with AdV-HPV6/11 (top row) or empty vector (bottom row) for responses against overlapping 15mer peptides from HPV 6 or 11 E6 or the minimal shared epitope IYSYAYKHLK (SEQ ID NO: 120). PMA/Iono is positive control. FIG. 5 discloses SEQ ID NO: 120.

FIG. 6A depicts a diagram of the retroviral transduction to create MOC1 cells that express HPV6 E6.

FIG. 6B depicts flow cytometry dot plots demonstrating E6 (NGFR) positivity in parental MOC1 or MOC1-E6 cells, quantified on the right.

FIG. 6C depicts summary tumor growth curves of mice bearing parental MOC1 or MOC1-E6 tumors (n=7 mice/group) treated with AdV-HPV6/11, empty C46 or control (PBS). Red dots indicated treatments. Day 40 tumor volumes are individually quantified on the right.

FIG. 6D depicts summary growth curves of mice bearing MOC1-E6 tumors treated with AdV-HPV6/11 in the presence or absence of CD8 or CD4 depleting antibodies. Red dots indicate treatments and blue dots indicate depletions. Day 40 tumor volumes are individually quantified on the right.

FIG. 7A depicts representative dot plots of freshly digested parental pMOC1 or MOC1-E6 tumors obtained from mice treated with AdV-HPV6/11 or empty GC46 assessed for T-lymphocyte accumulation via flow cytometry. Normalized quantification is shown on the right.

FIG. 7B depicts representative H&E and immunofluorescence photomicrographs demonstrating localization of T lymphocytes within MOC1-E6 tumors treated with AdV-HPV6/11 or empty GC46.

FIG. 7C depicts a quantification of total T lymphocytes per high power field (HPF). mpIF multiplex immunofluorescence.

FIG. 7D depicts a quantification of tumor margin vs tumor parenchyma localization of CD8+ and CD4+ T lymphocytes.

FIG. 8A depicts a schematic of tumor-infiltrating lymphocytes (TIL) cultured from tumors from MOC1-E6 tumor-bearing mice treated with AdV-HPV6/11 or empty GC46 vector (n=5 mice/group).

FIG. 8B depicts a representative impedance analysis of cultured TIL from MOC1-E6 tumors treated with AdV-HPV6/11, empty GC46, or control (PBS), then co-cultured with either parental MOC1 or MOC1-E6 target tumor cells (n=5 tumors per condition). TIL are added to target cells at experimental time 0. Percent killing (represented as % loss of target cell index) is quantified 16 h after the addition of TIL to targets and is shown on the right.

FIG. 8C depict photomicrographs of representative ELISpot wells and quantification of IFNγ spots demonstrating responses to HPV6 and 11 overlapping 15-mer peptide pools as well as synthesized minimal peptides in TIL cultures from MOC1-E6 tumors treated with AdV-HPV6/11 or empty GC46.

FIG. 9A depicts a bar plot showing the total number of clinically indicated interventions during the 12 months before (left) and after (right) the study for patients treated at DL1 (patients 1-3) and DL2 (patients 4-15). Responders are in blue and non-responders are in gold. Patients that require no interventions in the 12 months after study treatment are considered complete responders, and patients that require 50% or fewer interventions in the 12 months after the study treatment compared to the 12 months before are considered partial responders. Responders are complete and partial responders.

FIG. 9B depicts a waterfall plot showing the change in clinically indicated procedures in the year after the study treatment compared to the year before. Patients are rank ordered in magnitude of reduction in intervention frequency from left to right. The solid horizontal line indicates the threshold for a 50% reduction, separating responders from non-responders.

FIG. 9C depicts representative clinical endoscopy images that demonstrate the appearance of the larynx in 4 of 6 CRs that had no visible disease following AdV-HPV6/11 treatment. The Derkay score (upper-left) and timepoint after completion of the study treatment (upper-right) are inset. Post-treatment images are from the most recent endoscopy exam at the time of data cutoff.

FIG. 10A depicts a dot plot showing the log-transformed fold change in HPV-specific T cell responses from the peripheral blood 6 weeks after completing the study treatment compared to before. Each dot represents the log-transformed fold change in IFNγ concentration following peptide stimulation with an individual pool of HPV peptides encoded in AdV-HPV6/11 Pools to which IFNγ responses were not detected in the pre- or post-treatment samples are not shown. N=14 patients; insufficient pre-treatment PBMC were available from patient 5. Responders are in blue and non-responders are in gold.

FIG. 10B depicts a dot plot summarizing changes in HPV-specific peripheral blood responses. Significance was determined with a Mann-Whitney two-tailed test.

FIG. 10C depicts bar plots showing the fraction of the post-treatment TCRβ repertoire represented by the top 10 CDR3 frequencies determined to be HPV-specific in the HPV 6/11 peptide stimulation FEST assay. The Simpson clonality index is shown above each bar plot. Top horizontal bars indicate treatment response. R, responder; NR, non-responder.

FIG. 10D depicts a dot plot showing the log-transformed fold change in the frequency of the top 10 HPV-specific CDR3 sequences (determined in the FEST assay) from the peripheral blood 6 weeks after completing AdV-HPV6/11 treatment compared to before treatment without peptide stimulation.

FIG. 10E depicts a dot plot showing the summarized changes in the top 10 HPV-specific CDR3 sequences. Significance was determined with an unpaired two-tailed t-test.

FIG. 10F depicts bar plots summarizing the fraction of the top 10 peripheral blood HPV-specific CDR3 sequences (determined in the FEST assay) detected in the post-treatment peripheral blood that were undetectable (emergent), detected at lower frequencies (expanded) or detected and higher frequencies (contracted) compared to the pre-treatment peripheral blood. Top horizontal bars indicate treatment response.

FIG. 10G depicts a dot plot showing the log 2 transformed fold change in HPV-specific papilloma infiltrating lymphocytes (PIL) after completing the study treatment compared to before. Each dot represents the log-transformed fold change in IFNγ spot count following co-culture of PIL with antigen presenting cells loaded with an individual pool of HPV peptides encoded in AdV-HPV6/11. Pools to which IFNγ responses were not detected in the pre- or post-treatment samples are not shown. N=9 patients; no post-treatment biopsies were available for patients 7, 10, 11 and 13, and patients 2 and 3 failed to established pre-treatment PIL cultures.

FIG. 10H depicts a dot plot summarizing changes in HPV-specific PIL responses. Significance was determined with a Mann-Whitney two-tailed test.

FIG. 10I depicts clinical endoscopy images showing the pre-treatment and 6-week timepoint laryngeal appearance for patient 5. The red arrows indicate the pre-treatment and 6-week biopsy locations from which PIL were generated for experiments shown in FIG. 11J and FIG. 11K.

FIG. 10J depicts representative IFNγ ELISpot wells showing IFNγ spots following stimulation with pool 2 peptides (IFNγ spot counts are inset) in the pre-treatment and 6-week timepoints as well as negative (DMSO alone) and positive (PMA/lonomycin) controls.

FIG. 10K depicts a bar plot showing IFNγ concentrations following co-culture of the 6 weeks PIL sample with antigen presenting cells loaded with individual peptides included in pool 2.

FIG. 11A depicts representative photomicrographs of T cell immunofluorescence in baseline papilloma biopsies collected from responders (top row) and non-responders (bottom row)

FIG. 11B depicts dot plots show the density of CD8 T cells in the papilloma and stroma or responders and non-responders. Significance was determined with a Mann-Whitney two-tailed test.

FIG. 11C depicts box-and-whisker plots show papilloma cell normalized HPV gene transcript counts in responders and non-responders, determined from single-cell RNA-seq. Significance determined with a two-way ANOVA.

FIG. 11D depicts box-and-whisker plots show cell normalized reactome IFNγ signaling scores in different cell types (x-axis) in responders and non-responders, determined from single-cell RNA-seq. Significance determined with a two-way ANOVA.

FIG. 11E, heat maps show the row-normalized chemokine transcript counts in different cell types (y-axis). Bar plots on the right indicate mean expression. Top horizontal bars indicate treatment response. Significance of the difference between responders and non-responders was determined with a two-way ANOVA.

FIG. 11F depicts a violin plot showing the CXCR3 transcript counts for CD8 and CD4 papilloma T cells, determined from single-cell RNA-seq. Significance was determined with a Mann-Whitney two-tailed test.

FIG. 11G depicts a violin plot showing the percentage of (total) cells positive for CXCL9 or CXCL10. Significance was determined with a Mann-Whitney two-tailed test. Representative photomicrographs of RNAscope immunofluorescence are shown.

FIG. 11H depicts a dot plot showing the expression of select T cell-related genes across T lymphocyte clusters identified with single-cell RNA-seq, sorted by fold change in cell numbers detected in responders and non-responders (responders/non-responders; lower bar graph). T cells enriched in non-responders are in the left columns, T cells enriched in responders and in the right columns. Circle color corresponds to scaled average expression; circle size denotes fraction of cells with non-zero gene expression of corresponding gene. Top bar graph represents total cell number.

FIG. 12A depicts a dot plot showing the expression of select myeloid cell-related genes across myeloid clusters identified with single-cell RNA-seq, sorted by fold change in cell numbers detected in responders and non-responders (responders/non-responders; lower bar graph). Myeloid cells enriched in non-responders are in the left columns, myeloid cells enriched in responders and in the right columns. Circle color corresponds to scaled average expression; circle size denotes fraction of cells with non-zero gene expression of corresponding gene. Top bar graph represents total cell number.

FIG. 12B depicts heat maps showing the row-normalized chemokine transcript counts in different cell types (y-axis). Bar plots on the right indicate mean expression. Top horizontal bars indicate treatment response. Significance of the difference between responders and non-responders was determined with a two-way ANOVA.

FIG. 12C depicts a heat map showing the row-normalized VEGF transcript counts in different cell types (y-axis). Bar plots on the right indicate mean expression. Top horizontal bars indicate treatment response. Significance of the difference between responders and non-responders was determined with a two-way ANOVA.

FIG. 12D depicts a violin plot showing the percentage of (total) cells positive for CXCL8. Significance was determined with a Mann-Whitney two-tailed test. Representative photomicrographs of RNAscope immunofluorescence are shown.

FIG. 12E depicts representative photomicrographs of myeloid cell immunofluorescence in baseline papilloma biopsies are shown in responders (top row) and non-responders (bottom row).

FIG. 12F depicts dot plots showing the density of neutrophilic cells (PMN) and macrophages (M 0) in the papilloma and stroma or responders and non-responders. Significance was determined with a Mann-Whitney two-tailed test.

FIG. 13 depicts photomicrographs of H&E-stained slides from each of the 15 patients enrolled on this study are shown. Patient number is inset in the upper-left corner of each image.

FIG. 14 depicts a dot plot showing Derkay scores (y-axis) obtained from available clinical endoscopy images from the 12 months before the study, during the study, and the 12 months after the study (x-axis) are plotted for each patient. Lines color coded by response.

FIGS. 15A-15C depict representative pre- and post-treatment clinical endoscopy images of the larynx and trachea, if applicable, in (A) patients that were CRs but had visible disease remaining after treatment, (B) partial responders and (C) non-responders. The Derkay score is inset in the top left and the image timepoint is inset in the top right for each image. For the CRs, post-treatment images are from the most recent endoscopy exam at the time of data cutoff. For PRs and non-responders, the image is from the time of first clinically indicated procedure after completion of the study treatment.

FIG. 16A depicts representative immunofluorescence photomicrographs of T cell staining.

FIG. 16B depicts representative immunofluorescence photomicrographs of T cell phenotypes.

FIGS. 16C-16G depict the papilloma and stroma density of (C) Ki67+ CD8 T cells, (D) total CD4 T cells, (E) Ki67+ CD4 T cells, and (F) regulatory T cells (Tregs) in responders and non-responders. Papilloma cell PD-L1 H-scores are shown in (G). Significance was determined with a Mann-Whitney two-tailed test.

FIG. 17A depicts a scatter plot showing UMAP embedding of all sequenced cells from all 13 patients, annotated by cell type.

FIG. 17B depicts a bar plot showing average chemokine expression within monocytic or neutrophilic cells based upon single-cell RNA-seq.

FIG. 17C depicts representative immunofluorescence photomicrographs of chemokine RNAscope staining.

FIG. 17D depicts a scatter plot showing UMAP embedding of CD8 T cells with individual clusters identified by color.

FIG. 17E depicts a scatter plot showing UMAP embedding of CD4 T cells with individual clusters identified by color.

FIG. 17F depicts a dot plot showing the expression of select T cell-related genes across T lymphocyte clusters identified with single-cell RNA-seq, sorted by fold change in cell numbers detected in responders and non-responders (responders/non-responders; lower bar graph). T cells enriched in non-responders are in the left columns, T cells enriched in responders and in the right columns. Circle color corresponds to scaled average expression; circle size denotes fraction of cells with non-zero gene expression of corresponding gene. Top bar graph represents total cell number.

FIGS. 18A and 18B depict dot plots showing terms enriched in responders and non-responders upon gene set enrichment analysis of (A) all papilloma monocytic cells or (N) all papilloma neutrophilic cells. P-values were computed based on the hypergeometric distribution and adjusted using the Benjamini-Hochberg method.

FIG. 19A depicts a bar plot showing average chemokine expression within monocytic or neutrophilic cells based upon single-cell RNA-seq.

FIG. 19B depicts a representative immunofluorescence photomicrographs of myeloid cell marker staining.

FIG. 20 depicts bar graph results from a neutralizing antibody assay of serum samples collected from clinical study phase 1 participants' baseline (pre-treatment) and 43 days (6 weeks), 12 weeks, and 24 weeks post-treatment with AdV-HPV6/11, further categorized by participants' overall clinical response (complete response (CR), partial response (PR), or no response (NR)).

FIG. 21 depicts a dot plot of neutralizing HIV 6/11 antibody titers elicited in clinical study phase 1 participants pre-treatment (baseline) and post-treatment with DL1 (1×10¹¹ particle units) and DL2 (5×10¹¹ particle units) of AdV-HPV6/11.

FIG. 22 depicts the Phase II study protocol.

FIG. 23 depicts a graph of the number of surgeries in the past 12-months prior to AdV-HPV6/11 treatment compared to the number of surgeries in the 12-months post treatment.

FIG. 24 depicts a bar graph of the number of months since completing surgery and the number of pre-treatment surgeries in 12-months prior to Adv-HPV6/11 treatment. Each bar represents an individual patient who has not has surgery since treatment.

### DETAILED DESCRIPTION OF THE INVENTION

It is to be understood that the present disclosure is not limited to the particular embodiments described herein and as such can vary. Although various features of the disclosure can be described in the context of a single embodiment, the features can also be provided separately or in any suitable combination. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

### Definitions

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Use of the term “including” as well as other forms, such as “include,” “includes,” and “included,” is not limiting; i.e., “including” does not mean “limited to.”

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional possible components, elements, or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “isolated” and its grammatical equivalents as used herein refer to the removal of a nucleic acid, protein, polypeptide, cell, or other material from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Nucleic acid,” “nucleic acid molecule,” “polynucleotide,” “polynucleotide construct,” “oligonucleotide,” and their grammatical equivalents as used herein refer to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA It also includes modified, for example, by methylation and/or by capping, and unmodified forms of the polynucleotide. The term is also meant to include molecules that include non-naturally occurring, synthetic, and semi-synthetic nucleotides and polynucleotides as well as nucleotide analogs. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant polynucleotide” is a polynucleotide that has undergone a molecular biological manipulation. The polynucleotide sequences and vectors disclosed or contemplated herein can be introduced into a cell by, for example, transfection, transformation, or transduction.

The term “fragment,” as applied to a polynucleotide or nucleic acid sequence, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000, or more consecutive nucleotides of a nucleic acid according to the invention.

As used herein, an “isolated polynucleotide” or “isolated nucleic acid fragment” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “gene” and its grammatical equivalents refers to a polynucleotide comprising nucleotides that encode a functional molecule, including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genome” includes chromosomal as well as mitochondrial, chloroplast and viral DNA or RNA. The term “probe” refers to a single-stranded nucleic acid molecule that can base pair with a complementary single stranded target nucleic acid to form a double-stranded molecule.

“Heterologous DNA” refers to DNA not naturally located in the cell, or in a chromosomal site of the cell. The heterologous DNA may include an exogenous gene. “Exogenous gene” means a gene foreign to the subject, that is, a gene which is introduced into the subject through a transformation process, an unmutated version of an endogenous mutated gene or a mutated version of an endogenous unmutated gene. Exogenous genes can be either natural or synthetic genes which are introduced into the subject in the form of DNA or RNA which may function through a DNA intermediate such as by reverse transcriptase. Such genes can be introduced into target cells, directly introduced into the subject, or indirectly introduced by the transfer of transformed cells into the subject.

A “primer” refers to an oligonucleotide that hybridizes to a target nucleic acid sequence to create a double stranded nucleic acid region that can serve as an initiation point for DNA synthesis under suitable conditions. Such primers may be used in a polymerase chain reaction or for DNA sequencing.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxy) terminus. A coding sequence can include, but is not limited to, prokaryotic sequences, cDNA from mRNA, genomic DNA sequences, and even synthetic DNA sequences. If the coding sequence is intended for expression in an eukaryotic cell, a polyadenylation signal and transcription termination sequence will usually be located 3′ to the coding sequence.

“Open reading frame” is abbreviated ORE and refers to a length of nucleic acid sequence, either DNA, cDNA or RNA, that comprises a translation start signal or initiation codon, such as an ATG or AUG, and a termination codon and can be potentially translated into a polypeptide sequence.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

The term “response element” refers to one or more cis-acting DNA elements which confer responsiveness on a promoter mediated through interaction with the DNA-binding domains of a transcription factor. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds, in the presence or absence of a ligand, to the DNA sequence of a response element to initiate or suppress transcription of downstream gene(s) under the regulation of this response element. Examples of DNA sequences for response elements of the natural ecdysone receptor include: RRGG/TTCANTGAC/ACYY (see Cherbas et. al., Genes Dev. 1991); AGGTCAN(n)AGGTCA, where N(n) can be one or more spacer nucleotides (see D'Avino et al., Mol. Cell. Endocrinol. 113:1 1995); and GGGTTGAATGAATTT (see Antoniewski et al., Mol. Cell Biol. 14:4465 1994).

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

As used herein, the term “codon degenerate variant” refers to a modified nucleic acid sequence that encodes the same amino acid sequence as the original sequence but differs in the specific nucleotides comprising the codons. The genetic code is degenerate, meaning that multiple codons can code for the same amino acid. For example, the amino acid leucine can be encoded by six different codons: CTG, CTT, CTC, CTA, TTG, and TTA. A codon degeneracy table, also known as a genetic code table or codon table, is a chart that provides information about the relationship between codons (sequences of three nucleotides) and the corresponding amino acids they encode. The table lists the 64 possible codons and indicates which amino acid each codon represents. Table 1 is an example of a codon degeneracy table:

TABLE 1
Codon Degeneracy Table
Nucleotide Position in Codon - Amino Acid Encoded

| First | Second nucleotide | | | | Third |
|---|---|---|---|---|---|
| nucleotide | U | C | A | G | nucleotide |
| U | UUU - Phe | UCU - Ser | UAU - Tyr | UGU - Cys | U |
| | UUC - Phe | UCC - Ser | UAC - Tyr | UGC - Cys | C |
| | UUA - Leu | UCA - Ser | UAA - * | UGA - * | A |
| | UUG - Leu | UCG - Ser | UAG - * | UGG - Trp | G |
| C | CUU - Leu | CCU - Pro | CAU - His | CGU - Arg | U |
| | CUC - Leu | CCC - Pro | CAC - His | CGC - Arg | C |
| | CUA - Leu | CCA - Pro | CAA - Gln | CGA - Arg | A |
| | CUG - Leu | CCG - Pro | CAG - Gln | CGG - Arg | G |
| A | AUU - Ile | ACU - Thr | AAU - Asn | AGU - Ser | U |
| | AUC - Ile | ACC - Thr | AAC - Asn | AGC - Ser | C |
| | AUA - Ile | ACA - Thr | AAA - Lys | AGA - Arg | A |
| | AUG - Met | ACG - Thr | AAG - Lys | AGG - Arg | G |
| G | GUU - Val | GCU - Ala | GAU - Asp | GGU - Gly | U |
| | GUC - Val | GCC - Ala | GAC - Asp | GGC - Gly | C |
| | GUA - Val | GCA - Ala | GAA - Glu | GGA - Gly | A |
| | GUG - Val | GCG - Ala | GAG - Glu | GGG - Gly | G |

(U indicating “Uracil” as incorporated into mRNA in place of T (Thymine) as found in DNA; both of which form complementary base-pairs with “A” (Adenine); “*” indicates stop codons.)

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

Furthermore, publicly available software resources are readily available for computer-generated “reverse-translation,” also known as, “back translation” of polypeptide sequences, i.e., converting polypeptide sequences into nucleotide sequences encoding same). See, e.g., Madeira, F., et al., Nucleic Acids Res, 47(W1), W636-W641 (2019); Madeira, F., et al., Curr Protoc in Bioinformatics, 66(1):e74 (2019); Chojnacki, S, et al., Nucleic Acids Res. 2017 Jul. 3; 45(W1):W550-W553 (2017); Athey, J., et al., BMC Bioinformatics 18:391 (2017).

As used herein, a codon degenerate variant may be utilized to optimize gene expression or enhance protein production. By modifying the codons within a nucleic acid sequence, it is possible to utilize codons that are more frequently used or preferred by the host organism's translational machinery. This can lead to increased efficiency in protein expression or improved compatibility with a specific host organism.

The term “expression” as used herein refers to the transcription and stable accumulation of sense (mRNA) or antisense RNA derived from a nucleic acid or polynucleotide. Expression may also refer to translation of mRNA into a protein or polypeptide.

The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like. The expression cassettes described herein may comprise a total length of between about 500 to about 10,000 bp, about 1,000 to about 5,000 bp, about 1,500 to about 4,500 bp, about 1,800 to about 4,400 bp, about 2,000 to about 4,500 bp, about 2,100 to about 4,400 bp, about 2,200 to about 4,300 bp, about 2,300 to about 4,200 bp, about 2,400 to about 4,100 bp, about 2,500 to about 4,000 bp, about 2,600 to about 3,900 bp, about 2700 to about 3800 bp, about 2,800 to about 3,800 bp, about 2,900 to about 3,700 bp, about 3,000 to about 3,600 bp, about 3,100 to about 3,500 bp, about 3,150 to about 3,450 bp, about 3,200 to about 3,400 bp, about 3,250 to about 3,350 bp, or about 3300 bp. Alternatively, the expression cassette may comprise any number of base pairs falling within these ranges. For instance, the expression cassette may comprise about 500 bp, about 750 bp, about 1,000 bp, about 1,250 bp, about 1,500 bp, about 1,750 bp, about 2,000 bp, about 2,250 bp, about 2,500 bp, about 2,550 bp, about 2,600 bp, about 2,650 bp, about 2,700 bp, about 2,750 bp, about 2,800 bp, about 2,850 bp, about 2,900 bp, about 2,950 bp, about 3000 bp, about 3,050 bp, about 3,100 bp, about 3,150 bp, about 3,200 bp, about 3,250 bp, about 3,300 bp, about 3,350 bp, about 3,400 bp, about 3450 bp, about 3,500 bp, about 3,550 bp, about 3,600 bp, about 3,650 bp, about 3,700 bp, about 3,750 bp, about 3,800 bp, about 3,850 bp, about 3,900 bp, about 3,950 bp, about 4,000 bp, about 4,050 bp, about 4,100 bp, about 4,150 bp, about 4,200 bp, about 4,250 bp, about 4,300 bp, about 4,350 bp, about 4,400 bp, about 4,450 bp, or about 4,500 bp. In one aspect, the expression cassette comprises about 2,800 bp. In another aspect, the expression comprises 2,825 bp.

As used herein, the term “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both, viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. Another example of vectors that are useful in the invention is the ULTRAVECTOR® Production System (Intrexon Corp., Blacksburg, VA) as described in WO 2007/038276. For example, the insertion of the DN fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

As used herein, the term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the terms “cloning vector” and “replicon” refer to a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

As used herein, the term “viral vector” as used herein refers to a virus, viral particle, or derivative thereof, capable of transferring a nucleic acid into a cell or to the transferred nucleic acid itself. Viral vectors and transfer plasmids contain structural and/or functional genetic elements that are primarily derived from a virus. Viral vectors, and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

As used herein, the term “adenovirus” and “adenoviral vector” as used herein, refers to an adenovirus that retains the ability to participate in the adenovirus life cycle and/or which has been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5^{th} ed., Knipe et al. (eds.), Lippincott Williams & Wilkins, Philadelphia, PA (2006)). Adenoviruses, as used and described herein may also be rendered replication deficient (i.e., do not retain ability to participate in the adenovirus life cycle) by deletion of one or more parts of the naturally occurring viral genome. “Adenoviruses” and “Adenoviral vector,” as used and described herein, may include an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

As used herein, the terms “MOI” or “Multiplicity of Infection” refer to the average number of virus particles that infect a single cell in a specific experiment (e.g., recombinant virus or control virus).

As used herein, the term “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, the term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, the term “electroporation” refers to the use of a transmembrane electric field pulse to transiently increase the permeability of a cell membrane, allowing the introduction of exogenous biological materials, such as DNA, RNA, peptides, polypeptides, proteins, enzymes, or ribonucleoproteins (RNPs), into the cell. The electric field pulse creates transient pores in the cell membrane, facilitating the uptake of the biological material. Electroporation can be performed using specialized buffers and devices that control the pH, conductivity, osmolality, and other parameters to optimize the process and enhance transfection efficiency while minimizing cell damage. Electroporation can be used to introduce exogenous materials (e.g., biological molecules, plasmids, oligonucleotides, expression cassettes, siRNA, drugs, and ions) into various cell types, including primary human blood cells, immune cells, pluripotent precursor cells, fibroblasts, and endothelial cells, for applications in gene therapy, cell therapy, and biotechnology research.

As used herein, the term terms “induce,” “induction” and their grammatical equivalents as used herein refer to an increase in nucleic acid sequence transcription, promoter activity and/or expression brought about by a transcriptional regulator, relative to some basal level of transcription.

As used herein, the terms “promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease SI), as well as protein binding domains (consensus sequences) responsible for the binding of RNA polymerase.

The source of the promoter can be natural or synthetic, and the source of the promoter should not limit the scope of the invention described herein. In other words, the promoter may be directly cloned from cells, or the promoter may have been previously cloned from a different source, or the promoter may have been synthesized.

As used herein, the term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

As used herein, the term “enhancer” refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

As used herein, the term “therapeutic switch promoter” (“TSP”) refers to a promoter that controls expression of a gene switch component. Gene switches and their various components are described in detail elsewhere herein. In certain embodiments a TSP is constitutive, i.e., continuously active. A constitutive TSP may be either constitutive-ubiquitous (i.e., generally functions, without the need for additional factors or regulators, in any tissue or cell) or constitutive-tissue or cell specific (i.e., generally functions, without the need for additional factors or regulators, in a specific tissue type or cell type). In certain embodiments a TSP of the invention is activated under conditions associated with a disease, disorder, or condition. In certain embodiments of the invention where two or more TSPs are involved the promoters may be a combination of constitutive and activatable promoters. As used herein, a “promoter activated under conditions associated with a disease, disorder, or condition” includes, without limitation, disease-specific promoters, promoters responsive to particular physiological, developmental, differentiation, or pathological conditions, promoters responsive to specific biological molecules, and promoters specific for a particular tissue or cell type associated with the disease, disorder, or condition, e.g. tumor tissue or malignant cells. TSPs can comprise the sequence of naturally occurring promoters, modified sequences derived from naturally occurring promoters, or synthetic sequences (e.g., insertion of a response element into a minimal promoter sequence to alter the responsiveness of the promoter).

Therapeutic switch promoters useful in the invention may include any promoter that is useful for treating, ameliorating, or preventing a specific disease, disorder, or condition. Examples include, without limitation, promoters of genes that exhibit increased expression only during a specific disease, disorder, or condition and promoters of genes that exhibit increased expression under specific cell conditions (e.g., proliferation, apoptosis, change in pH, oxidation state, oxygen level). In some embodiments where the gene switch comprises more than one transcription factor sequence, the specificity of the therapeutic methods can be increased by combining a disease- or condition-specific promoter with a tissue- or cell type-specific promoter to limit the tissues in which the therapeutic product is expressed. Thus, tissue- or cell type-specific promoters are encompassed within the definition of therapeutic switch promoter.

The term “ecdysone receptor-based,” with respect to a gene switch, refers to a gene switch comprising at least a functional part of a naturally occurring or synthetic ecdysone receptor ligand binding domain and which regulates gene expression in response to a ligand that binds to the ecdysone receptor ligand binding domain. Examples of ecdysone-responsive systems are described in U.S. Pat. Nos. 7,091,038 and 6,258,603. Additional examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. In one embodiment, the system is the RheoSwitch® Therapeutic System (RTS), which contains two fusion proteins, the DEF domains of a mutagenized ecdysone receptor (EcR) fused with a Gal4 DNA binding domain and the EF domains of a chimeric RXR fused with a VP16 transcription activation domain, expressed under a constitutive promoter.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” refer to DNA regulatory sequences, such as promoters, enhancers, terminators, and the like, that provide for the expression of a coding sequence in a host cell. In eukaryotic cells, polyadenylation signals are control sequences. Enhancers that may be used in embodiments of the invention include but are not limited to: an SV40 enhancer, a cytomegalovirus (CMV) enhancer, an elongation factor 1 (EF 1) enhancer, yeast enhancers, viral gene enhancers, and the like.

The terms “3′ non-coding sequences” and “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

As used herein, the term “regulatory region” refers to a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

As used herein, the term “modulate” means to induce, reduce or inhibit nucleic acid or gene expression, resulting in the respective induction, reduction or inhibition of protein or polypeptide production.

As used herein, the term “inducible promoter” refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch. The inducible promoter can be a gene switch ligand inducible promoter. In some cases, an inducible promoter can be a small molecule ligand-inducible two polypeptide ecdysone receptor-based gene switch. In some cases, a gene switch can be selected from ecdysone-based receptor components as described in, but without limitation to, any of the systems described in: International Patent Applications WO 2001/070816; WO 2002/029075; WO 2002/066613; WO 2002/066614; WO 2002/066612; WO 2002/066615; WO 2003/027266; WO 2003/027289; WO 2005/108617; WO 2009/045370; WO 2009/048560; WO 2010/042189; WO 2010/042189; WO 2011/119773; and WO 2012/122025; and U.S. Pat. Nos. 7,091,038; 7,776,587; 7,807,417; 8,202,718; 8,105,825; 8,168,426; 7,531,326; 8,236,556; 8,598,409; 8,715,959; 7,601,508; 7,829,676; 7,919,269; 8,030,067; 7,563,879; 8,021,878; 8,497,093; 7,935,510; 8,076,454; 9,402,919; 9,493,540; 9,249,207; and 9,492,482, each of which is incorporated by reference in its entirety.

As used herein, two or more individually operable gene regulation systems are said to be “orthogonal” when: a) modulation of each of the given systems by its respective ligand, at a chosen concentration, results in a measurable change in the magnitude of expression of the gene of that system, and b) the change is statistically significantly different than the change in expression of all other systems simultaneously operable in the cell, tissue, or organism, regardless of the simultaneity or sequentiality of the actual modulation. Preferably, modulation of each individually operable gene regulation system effects a change in gene expression at least 2-fold greater than all other operable systems in the cell, tissue, or organism, e.g., at least 5-fold, 10-fold, 100-fold, or 500-fold greater. Ideally, modulation of each of the given systems by its respective ligand at a chosen concentration results in a measurable change in the magnitude of expression of the gene of that system and no measurable change in expression of all other systems operable in the cell, tissue, or organism. In such cases the multiple inducible gene regulation system is said to be “fully orthogonal.” Useful orthogonal ligands and orthogonal receptor-based gene expression systems are described in US 2002/0110861 A1.

As used herein, the term “gene switch” as used herein refers to the combination of a response element associated with a promoter, and a ligand-dependent transcription factor-based system which, in the presence of one or more ligands, modulates the expression of a gene into which the response element and promoter are incorporated. The term “a polynucleotide encoding a gene switch” refers to the combination of a response element associated with a promoter, and a polynucleotide encoding a ligand-dependent transcription factor-based system which, in the presence of one or more ligands, modulates the expression of a gene into which the response element and promoter are incorporated. Tightly regulated inducible gene expression systems or gene switches, such as EcR based systems, are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

As used herein, the term “CAP” or “cap” refers to a modified nucleotide, generally a 7-methyl guanosine, linked 3′ to 5′ (7meG-ppp-G), to the 5′ end of a eukaryotic mRNA, that serves as a required element in the normal translation initiation pathway during expression of protein from that mRNA.

As used herein, the term “Sleeping Beauty (SB) Transposon System” refers a synthetic DNA transposon system for to introducing DNA sequences into the chromosomes of vertebrates. Some exemplary embodiments of the system are described, for example, in U.S. Pat. Nos. 6,489,458, 8,227,432, 9,228,180 and WO/2016/145146. The Sleeping Beauty transposon system is composed of a Sleeping Beauty (SB) transposase and a SB transposon.

In embodiments, the Sleeping Beauty transposon system can include the SB11 transposon system, the SB100X transposon system, or the SB110 transposon system.

As used herein, the term “transposon” or “transposable element” (TE) refers to a vector DNA sequence that can change its position within the genome, sometimes creating or reversing mutations and altering the cell's genome size. Transposition often results in duplication of the TE. Class I TEs are copied in two stages: first they are transcribed from DNA to RNA, and the RNA produced is then reverse transcribed to DNA This copied DNA is then inserted at a new position into the genome. The reverse transcription step is catalyzed by a reverse transcriptase, which can be encoded by the TE itself The characteristics of retrotransposons are similar to retroviruses, such as HIV. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons can be identified by short direct repeats which can be created by the staggered cut in the target DNA and filling in by DNA polymerase, followed by a series of inverted repeats important for the TE excision by transposase. Cut-and-paste TEs can be duplicated if their transposition takes place during S phase of the cell cycle when a donor site has already been replicated, but a target site has not yet been replicated.

Transposition can be classified as either autonomous or non-autonomous in both Class I and Class II TEs. Autonomous TEs can move by themselves while non-autonomous TEs require the presence of another TE to move. This is often because non-autonomous TEs lack transposase (for class II) or reverse transcriptase (for class I).

As used herein, the term “transposase” refers to an enzyme that binds to the end of a transposon and catalyzes the movement of the transposon to another part of the genome by a cut and paste mechanism or a replicative transposition mechanism.

As used herein, the terms “polypeptide,” “peptide,” “polypeptide construct,” and “peptide construct” and their grammatical equivalents, refer to a polymeric compound comprised of covalently linked amino acid residues. A “mature protein” is a protein which is full-length and which, optionally, includes glycosylation or other modifications typical for the protein in a given cellular environment. As disclosed herein, embodiments of the invention include HPV antigens/antigenic polypeptides, peptides, and mature proteins described herein and also polynucleotides (DNA or RNA) that encode the same. Polypeptides and proteins disclosed herein (including functional fragments and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine.

As used herein, the term “polypeptide fragment” refers to a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of at least 2, 3, 4, 5, 6, 8, 10, 13, 14, 15, 16, 17, 18, 19, 20, 21, 22, 25, 26, 30, 35, 40, 45, 50, 100, 200, 240, or 300 or more amino acids.

As used herein, the terms “isolated polypeptide,” “isolated peptide,” or “isolated protein” refer to a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be, for example, due to incomplete purification, addition of stabilizers, or compounding into a pharmaceutically acceptable preparation.

As used herein, the term “identical” or “sequence identity” in the context of two nucleic acid sequences or amino acid sequences of polypeptides refers to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J Mal. Biol., 48:443 (1970); by the search for similarity method of Pearson and Lipman, Proc. Nat. Acad Sci USA., 85:2444 (1988); by computerized implementations of these algorithms (including, but not limited to CLUSTAL in the PC/Gene program by Intelligentics, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment.

In one class of embodiments, the polypeptides herein are at least 80%, 85%, 90%, 98%, 99%, 99.1%, 99.5%, 99.9%, 99.99%, or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99%, 99%, 99.1%, 99.5%, 99.9%, 99.99%, or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

As used herein, the term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including but not limited to those described above or in, e.g., Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M, and Devereux, J., eds.) Stockton Press, New York (1991). Methods to determine identity and similarity are codified in publicly available computer programs. Sequence alignments and percent identity calculations may be performed using sequence analysis software such as the MegAlign (or more recently MegAlign Pro) program of the LASERGENE bioinformatics computing suite (DNASTAR Inc., Madison, Wis.). Multiple alignment of the sequences may be performed using a Clustal method of alignment (Higgins et al., CABIOS. 5:151 1989) with the default parameters (GAP PENALTY=10, GAP LENGTH PENALTY=10). Default parameters for pairwise alignments using a Clustal method may be selected: KTUPLE 1, GAP PENALTY=3, WINDOW=5 and DIAGONALS SAVED=5.

As used herein, the term “substantially similar” and its grammatical equivalents as applied to nucleic acid or amino acid sequences mean that a nucleic acid or amino acid sequence comprises a sequence that has at least 90% sequence identity or more, such as at least 95%, at least 98%, at least 99%, at least 99.1%, at least 99.2%, at least 99.3%, at least 99.4%, at least 99.5%, at least 99.6%, at least 99.7%, at least 99.8%, at least 99.9%, and at least 99.99% sequence identity to a reference sequence using the comparison programs described above, e.g., BLAST, using standard parameters. The term “substantially identical” and its grammatical equivalents as applied to nucleic acid or amino acid sequences mean that a nucleic acid or amino acid sequence comprises a sequence that has at least 99%, such as at least at least 99.1%, at least 99.2%, at least 99.3%, at least 99.4%, at least 99.5%, at least 99.6%, at least 99.7%, at least 99.8%, at least 99.9%, and at least 99.99% sequence identity to a reference sequence using the comparison programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

As used herein, the term “functional fragment,” or its grammatical equivalents, is used herein to mean a portion, fragment, or segment of a biological molecule that retains the essential functional characteristics or activities of the original biological molecule. The term “functional variant,” or its grammatical equivalents, is used herein to mean a modified form of a biological molecule that retains the essential functional characteristics or activities of the original molecule while exhibiting some degree of variation. It includes a biological molecule that has been altered, such as through genetic engineering or mutagenesis techniques, to introduce specific changes while preserving the biological molecule's overall functionality. A functional variant may have one or more amino acid substitutions, insertions, or deletions compared to the original molecule, while still maintaining the desired biological activity or function. The techniques for obtaining these variants, including genetic (suppressions, deletions, mutations, etc.), chemical, and enzymatic techniques, are known to persons having ordinary skill in the art. In one embodiment, a variant biological comprises at least about 14 monomers (e.g., nucleotides or amino acids).

As used herein, the term “homology” in all its grammatical forms and spelling variations refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

The term “substitution,” when used in the context of an amino acid sequence refers to a variation wherein one amino acid in the amino acid sequence is replaced by another. The nomenclature used to denote amino acid substitutions follows a standardized format. Taking “L50G” as an example, “L” represents the amino acid leucine (abbreviated as “L”) at the original position, “50” signifies the position of the amino acid in the amino acid sequence in relation to the N-terminus thereof (in this case, the amino acid is the 50th amino acid from the N-terminus of the sequence), and “G” indicates the substituted amino acid, in this instance, glycine (abbreviated as “G”). Therefore, an “L50G” denotes a substitution where leucine at position 50 of the amino acid sequence (relative to the N-terminus thereof) has been replaced by glycine.

As used herein, the term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (see Schulz, G. E, and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E, and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Exemplary conservative amino acid substitutions are shown in the following chart:

TABLE 2
Exemplary conservative amino acid substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

An amino acid sequence that differs from a reference amino acid sequence by only conservative amino acid substitutions will be referred to herein as a “conservatively-substituted variant” of the reference sequence. Given the established knowledge and well-known techniques in protein science, it is well within the skill of a person of ordinary skill in the art to determine the functional impact of a “conservatively-substituted variant” as compared to the reference amino acid sequence.

In some embodiments, the functional variant may be a conservatively-substituted variant of the reference sequence. In some embodiments, the conservatively-substituted variant may differ from the amino acid sequence of the reference sequence by 100 or fewer conservative amino acid substitutions. In some embodiments, the conservatively-substituted variant may differ from the amino acid sequence of the reference sequence by 90 or fewer amino acid substitutions. In some embodiments, the conservatively-substituted variant may differ from the amino acid sequence of the reference sequence by 80 or fewer amino acid substitutions. In some embodiments, the conservatively-substituted variant may differ from the amino acid sequence of the reference sequence by 70 or fewer conservative amino acid substitutions. In some embodiments, the conservatively-substituted variant may differ from the amino acid sequence of the reference sequence by 60 or fewer conservative amino acid substitutions. In some embodiments, the conservatively-substituted variant may differ from the amino acid sequence of the reference sequence by 50 or fewer conservative amino acid substitutions. In some embodiments, the conservatively-substituted variant may differ from the amino acid sequence of the reference protein by 40 or fewer conservative amino acid substitutions. In some embodiments, the conservatively-substituted variant may differ from the amino acid sequence of the reference sequence by 30 or fewer conservative amino acid substitutions. In some embodiments, the conservatively-substituted variant may differ from the amino acid sequence of the reference sequence by 20 or fewer conservative amino acid substitutions. In some embodiments, the conservatively-substituted variant may differ from the amino acid sequence of the reference sequence by 10 or fewer conservative amino acid substitutions. In some embodiments, the conservatively-substitute variant may differ from the reference sequence by 9 or fewer, 8 or fewer, 7 or fewer, 6 or fewer, 5 or fewer, 4 or fewer, 3 or fewer, 2 or fewer, or 1 conservative amino acid substitutions. In some embodiments, the conservatively-substituted variant may differ from the amino acid sequence of the reference sequence by at least 100 and 150 conservative amino acid substitutions. In some embodiments, the conservatively-substituted variant may differ from the amino acid sequence of the reference sequence by at least 150 conservative amino acid substitutions.

An amino acid sequence that differs from a reference amino acid sequence by at least one non-conservative amino acid substitution will be referred to herein as a “non-conservatively-substituted variant” of the reference sequence. As used herein, the term “non-conservative amino acid substitution” refers to an amino acid substitution between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein. Amino acid substitutability is discussed in more detail, for example, in L. Y. Yampolsky and A. Stoltzfus, “The Exchangeability of Amino acids in Proteins,” Genetics 2005 August; 170(4):1459-1472. Given the established knowledge and well-known techniques in protein science, it is well within the skill of a person of ordinary skill in the art to determine the functional impact of a non-conservative amino acid substitution in a functional variant as compared to the reference amino acid sequence.

In some embodiments, the functional variant may differ from the amino acid sequence of the reference sequence by at least one non-conservative amino acid substitution. In some embodiments, the functional variant may differ from the amino acid sequence of the reference sequence by at least two, at least three, at least four, at least five, at least six, at least seven, at least eight, at least nine, or at least ten non-conservative amino acid substitutions. In some embodiments, the functional variant may differ from the amino acid sequence of the reference sequence by between ten and 20 non-conservative amino acid substitutions. In some embodiments, the functional variant may differ from the amino acid sequence of the reference sequence by between 21 and 30 non-conservative amino acid substitutions. In some embodiments, the functional variant may differ from the amino acid sequence of the reference sequence by between 31 and 40 non-conservative amino acid substitutions. In some embodiments, the functional variant may differ from the amino acid sequence of the reference sequence by between 41 and 50 non-conservative amino acid substitutions. In some embodiments, the functional variant may differ from the amino acid sequence of the reference sequence by between 51 and 60 non-conservative amino acid substitutions. In some embodiments, the functional variant may differ from the amino acid sequence of the reference sequence by between 61 and 70 non-conservative amino acid substitutions. In some embodiments, the functional variant may differ from the amino acid sequence of the reference sequence by between 71 and 80 non-conservative amino acid substitutions. In some embodiments, the functional variant may differ from the amino acid sequence of the reference sequence by between 81 and 90 non-conservative amino acid substitutions. In some embodiments, the functional variant may differ from the amino acid sequence of the reference sequence by between 91 and 100 non-conservative amino acid substitutions. In some embodiments, the functional variant may differ from the amino acid sequence of the reference sequence by at least 100 non-conservative amino acid substitutions.

As used herein, the term “antibody” refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDRI, CDR2, and CDR3, form the “hypervariable region” of an antibody, which is responsible for antigen binding.

As used herein, the term “functional antibody fragment” and “functional fragment of an antibody,” or their grammatical equivalents, are used interchangeably to mean a portion, fragment, or segment of the antibody that retains the essential functional characteristics or activities of the original antibody. In one embodiment, that activity is the ability to specifically bind to an antigen. (See, generally, Holliger et al., Nat. Biotech., 23(9):1126-1129 (2005)). The functional antibody fragment may comprise, for example, one or more CDRs, the variable region (or portions thereof), the constant region (or portions thereof), or combinations thereof. Non-limiting examples of functional antibody fragments include: (i) an antigen-binding fragment (Fab), which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a variable fragment (“Fv”) consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Functional antibody fragments are known in the art and are described in more detail in, e.g., U.S. Pat. No. 8,603,950.

As used herein, the term “antibody-like molecules” can be for example proteins that are members of the Ig-superfamily which are able to selectively bind a partner. MHC molecules and T cell receptors are such molecules. In one embodiment, the antibody-like molecule is a TCR. In one embodiment, the TCR has been modified to increase its MHC binding affinity.

As used herein, the term “antigen recognition moiety” or “antigen recognition domain” refers to a molecule or portion of a molecule that specifically binds to an antigen. In one embodiment, the antigen recognition moiety is an antibody, antibody-like molecule or fragment thereof and the antigen is a tumor antigen.

As used herein, the term “immune cells” includes dendritic cells, macrophages, neutrophils, mast cells, eosinophils, basophils, natural killer cells and lymphocytes (e.g., B and T cells).

As used herein, the terms “T cell” or “T lymphocyte” refer to a type of lymphocyte that plays a central role in cell-mediated immunity. They can be distinguished from other lymphocytes, such as B cells and natural killer cells (NK cells), by the presence of a T-cell receptor (TCR) on the cell surface. “TCRs” are protein molecules found on the surface of T cells, which are a type of white blood cell involved in the adaptive immune response. A TCR's variable domain contains the highly polymorphic loops referred to as complementarity determining regions (CDRs), which are responsible for binding to the peptide-presenting MHC. There are two major forms of TCRs: αβ TCR and TS TCR. Both forms consist of two protein chains, known as alpha (α) and beta (β) chains for αβ TCRs and gamma (γ) and delta (6) chains for γδ TCRs. These chains come together to form a heterodimeric structure. The majority of T cells in the human immune system express αβ TCRs. The α chain and β chain of αβ TCRs are encoded by separate gene segments, which undergo recombination during T cell development to generate diverse TCR specificities. The α and β chains each contain variable (V), diversity (D), and joining (J) gene segments, similar to the antibody gene rearrangement process. The combination of V, D, and J gene segments contributes to the unique antigen-binding specificity of the αβ TCR. The αβ TCR recognizes antigenic peptides presented in the context of major histocompatibility complex (MHC) molecules on the surface of antigen-presenting cells. In contrast to αβ TCRs, TS TCRs are less prevalent in the immune system but still play important roles. The γ and δ chains of γδ TCRs are also encoded by separate gene segments and undergo recombination during T cell development. The γδ TCR gene rearrangement process is distinct from that of αβ TCRs. TS T cells often exhibit a tissue-specific distribution and are found in epithelial tissues, such as the skin and gut. γδ TCRs can recognize a variety of antigens, including certain peptides and non-peptide molecules, independently of MHC presentation. Both αβ TCRs and TS TCRs participate in immune surveillance and response, but they have different functions and specificities. The αβ TCRs are predominantly involved in recognizing peptides presented by major histocompatibility complex (MHC) molecules, while γδ TCRs can have more diverse antigen recognition capabilities.

TCRs, and constructs encoding TCRs, that recognize MHC—antigen complexes, can be generated and introduced into T cells (known as TCR T cells), and the ensuing TCR-peptide-MHC interaction can be exploited to trigger an immune response. Greenbaum et al., Cancer Immunol Res 1 Nov. 2021; 9 (11): 1252-1261. There is an interest in using TCRs with higher than normal range of affinity for peptide-MHC antigens (type I), referred to as high affinity TCRs, to: 1) driving the activity of CD4 helper T cells (which do not have a CD8 co-receptor), or 2) developing soluble TCRs that can be used to directly target cells by attaching “effector” molecules (e.g., antibody Fc regions, toxic drugs, or antibody scFvs such as anti-CD 3 antibodies to form bispecific proteins) (Ashfield and Jakobsen, IDrugs, 9, 554-9 (2006); Foote and Eisen Proc Natl Acad Sci USA, 97:10679-81 (2000); Holler et al., Proc Natl Acad Sci USA, 97:5387-92 (2000); Molloy et al., Curr Opin Pharmacol, 5:438-43 (2005); Richman and Kranz, Biomol Eng, 24:361-73 (2007)). This approach may also overcome the problem faced by some cancer patients whereby their T cells do not express TCRs with sufficient specificity and binding affinity for the underlying tumor antigen. For example, over 300 MHC restricted T cell defined tumor antigens (Cheever et al., Clin Cancer Res. 2009; 15(17):5323-5337) have been identified. These tumor antigens include mutated peptides, differentiation antigens, and over-expressed antigens, all of which serve as targets for therapy. Since most cancer antigens described to date are derived from intracellular proteins that can only be targeted at the cell surface in the context of MHC molecules, TCRs are ideal candidates for therapy as they have evolved to recognize this class of antigens. Similarly, TCRs can detect peptides derived from viral proteins that have been naturally processed in infected cells and displayed on the cell surface by MHC molecules. However, patients with these diseases may not have an optimized TCR that binds and destroys infected cells. Finally, in methods with high specificity, TCRs may be used as receptor antagonists for autoimmune targets, or as delivery agents to immunosuppress local immune cell responses, thereby avoiding general immunosuppression.

As used herein, the term “T helper cells” (TH or Th cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH9, TH17, TH22 or TFH (T follicular helper cells), which secrete different cytokines to facilitate different types of immune responses. Signaling from the APCs directs T cells into particular subtypes.

As used herein, the term “cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

As used herein, the term “memory T cells” refers to a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (TcM cells) and two types of effector memory T cells (TEM cells and TEMRA cells). Memory cells can be either CD4+ or CD8+. Memory T cells typically express the cell surface proteins CD45RO, CD45RA and/or CCR7.

As used herein, the term “regulatory T cells” (Treg cells), formerly known as suppressor T cells, refer to T cells that play a role in the maintenance of immunological tolerance. Their major role is to shut down T cell-mediated immunity toward the end of an immune reaction and to suppress autoreactive T cells that escaped the process of negative selection in the thymus.

As used herein, the term “Natural killer T cells” (NKT cells—not to be confused with natural killer cells of the innate immune system) refer to those cells that bridge the adaptive immune system with the innate immune system. Unlike conventional T cells that recognize peptide antigens presented by major histocompatibility complex (MHC) molecules, NKT cells recognize glycolipid antigen presented by a molecule called CD Id. Once activated, these cells can perform functions ascribed to both T helper (TH) and cytotoxic T (TC) cells (i.e., cytokine production and release of cytolytic/cell killing molecules). They are also able to recognize and eliminate some tumor cells and cells infected with herpes viruses.

As used herein, the term “proliferative disease” refers to a unifying concept in which excessive proliferation of cells and/or turnover of cellular matrix contributes significantly to the pathogenesis of the disease, including cancer.

“Patient” or “subject” as used herein refers to a mammalian subject diagnosed with or suspected of having or developing a disease or disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to human papilloma virus (HPV) infection.

“Administering” is referred to herein as providing one or more compositions described herein to a patient or a subject. By way of example and not limitation, composition administration, e.g., injection, can be performed by intravenous injection, subcutaneous injection, intradermal injection, intraperitoneal injection, or intramuscular injection. One or more such routes can be employed. Parenteral administration can be, for example, by bolus injection or by gradual perfusion over time. Alternatively, or concurrently, administration can be by the oral route. Additionally, administration can also be by surgical deposition, or positioning of a medical device. A pharmaceutical composition can comprise a composition of the invention as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “therapeutic product” refers to a therapeutic polypeptide or therapeutic polynucleotide which imparts a beneficial function to the host cell in which such product is expressed. Therapeutic polypeptides may include, without limitation, peptides as small as three amino acids in length, single- or multiple-chain proteins, and fusion proteins. Therapeutic polynucleotides may include, without limitation, antisense oligonucleotides, small interfering RNAs, ribozymes, and RNA external guide sequences. The therapeutic product may comprise a naturally occurring sequence, a synthetic sequence or a combination of natural and synthetic sequences.

As used herein, the term “treatment,” “treating,” or its grammatical equivalents refers to obtaining a desired pharmacologic and/or physiologic effect. In embodiments, the effect is therapeutic, i.e., the effect partially or completely cures a disease and/or adverse symptom or pathological manifestation attributable to the disease. To this end, the inventive method comprises administering a therapeutically effective amount of a composition of the invention expressing the inventive nucleic acid sequence, or a vector comprising the inventive nucleic acid sequences.

As used herein, a “treatment interval” refers to a treatment cycle, for example, a course of administration of a therapeutic agent that may be repeated, e.g., on a regular schedule. In some embodiments, a dosage regimen may have one or more periods of no administration of the therapeutic agent in between treatment intervals.

As used herein, a “dosage regimen” or “dosing regimen” includes a treatment regimen based on a determined set of doses. The terms “dose” and “dosing” as used herein refers to the administration of a substance to achieve a therapeutic objective (e.g., the treatment of a tumor).

The terms “administered in combination,” “co-administration,” or “co-administering,” or “co-providing” as used herein means that two (or more) different treatments are delivered to the subject during the course of the subject's affliction with a disease or disorder, for example, the two or more treatments are delivered after the subject has been diagnosed with the disease or disorder and before the disease or disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered.

In some embodiments of the present invention, a first treatment and a second treatment may be administered simultaneously (e.g., at the same time), in the same or in separate compositions, or sequentially. Sequential administration refers to administration of one treatment before (e.g., immediately before; less than 5, 10, 15, 30, 45, or 60 minutes before; 1, 2, 3, 4, 6, 8, 10, 12, 16, 20, 24, 48, 72, 96 or more hours before; 4, 5, 6, 7, 8, 9 or more days before; or 1, 2, 3, 4, 5, 6, 7, 8 or more weeks before) administration of an additional (e.g., secondary) treatment. The order of administration of the first and secondary treatment may also be reversed.

The term “therapeutically effective amount,” “therapeutic amount,” “immunologically effective amount,” “anti-tumor effective amount,” “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects.

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease or prevention of manifestation of a target pathology).

The term “Derkay score” refers to a scoring system used to assess the severity of pediatric recurrent respiratory papillomatosis (RRP). The Derkay score assigns points based on factors such as age of onset, frequency of surgeries, location of papillomas, and tracheostomy dependence. It helps clinicians gauge the extent of disease and guide treatment decisions. Higher scores indicate more severe cases requiring more aggressive management. See Hester R P, Derkay C S, Burke B L, Lawson M L: Reliability of a staging assessment system for recurrent respiratory papillomatosis. Int J Pediatr Otorhinolaryngol. 2003; 67(5):505-9; Derkay C S: Recurrent respiratory papillomatosis. Laryngoscope. 2001; 111(i):57-69.

### I. Vectors

Genetic therapy involving the introduction of a transgene (e.g., via vaccination) expressing an exogenous protein to a subject has proven useful in the treatment of diseases and disorders in subjects in need thereof. For such introduction, a vector, for example, a viral vector, comprising a transgene encoding such a protein is typically used.

The present invention is directed in part to a vector comprising an expression cassette, the expression cassette comprising a transgene encoding an HPV antigen design.

In certain embodiments, the vector is a plasmid.

Another suitable vector is an integrating expression vector. Such vectors are able to randomly integrate into the host cell's DNA, or can include a recombination site to enable the specific recombination between the expression vector and the host cell's chromosome. Such integrating expression vectors can utilize the endogenous expression control sequences of the host cell's chromosomes to effect expression of the desired protein. Examples of vectors that integrate in a site-specific manner include, for example, components of the flp-in system from Invitrogen (Carlsbad, Calif.) (e.g., pcDNATM5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.).

A. Methods for Introducing Nucleic Acids into Cells

Methods of introducing and expressing genes into a cell are well known. In the context of an expression vector, the vector can be readily introduced into a host cell, e.g., mammalian, bacterial, yeast, or insect cell by any method in the art. For example, the expression vector can be transferred into a host cell by physical, chemical, or biological means. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### 1. Physical Methods

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, Cold Spring Harbor Laboratory, New York (2001). In embodiments, a method for the introduction of a polynucleotide into a host cell is calcium phosphate transfection or polyethylenimine (PEI) Transfection.

In some embodiments, a method for introduction of a polynucleotide into a host cell is electroporation. Electroporation is a technique that uses electrical pulses to temporarily increase the permeability of cell membranes, allowing the uptake of nucleic acid molecules into the cells. This process enhances the delivery and expression of a biological material (e.g., peptide or nucleic acid) in the subject's cells, potentially improving the immune response against HPV. In some embodiments, the biological material is an HPV antigen. In other embodiments, the biological material is an HPV antigen-encoding nucleic acid.

Electroporation buffers may contain water, sugars, sugar alcohols, chloride salts, and buffering agents. The pH, conductivity, and osmolality of the buffer are carefully controlled. The buffer may be used with an UltraPorator™ electroporation apparatus and cartridge. The UltraPorator™ electroporation apparatus is designed for rapid manufacturing of gene and cell therapies and may be used as a scale-up and commercialization solution for decentralized cell manufacturing. See, e.g., PCT/US20/59984 (filed Nov. 11, 2020) and U.S. patent application Ser. No. 17/095,028 (filed Nov. 11, 2020).

In some embodiments, a suspension is formed by combining cells obtained from a human with an exogenous biological material in the buffer, and then an electric current is applied to the suspension to facilitate the introduction of the biological material into the cells. The voltage pulse may have a field strength of 1-10 kV/cm, a duration of 5-250 s, and a current density of at least 2 A/cm². The method can be used to introduce biological materials, such as nucleic acids, peptides, polypeptides, proteins, enzymes, or RNPs, into primary human blood cells, pluripotent precursor cells, fibroblasts, and endothelial cells. In some embodiments, the method is used to introduce biologically active material into primary human blood cells, pluripotent precursor cells of human blood, as well as primary human fibroblasts and endothelial cells. In some embodiments, the cells are human blood cells, for example immune cells. In certain embodiments, the immune cells are neutrophils, eosinophils, basophils, mast cells, monocytes, macrophages, dendritic cells, natural killer cells, and lymphocytes (B cells and T cells), or some combination thereof. In some embodiments, the lymphocytes are T-cells. In certain embodiments, the cells are obtained from a patient.

In some embodiments, the transfection yield and transfected cell recovery yield using the electroporation buffer may be significantly higher than those obtained using control buffers. In some embodiments, the transfection yield with a buffer of the invention is at least about 1.1 times that of the transfection yield with a control buffer, for example, 1.1, 1.2, 1.3, 1.4, 1.5, 1.6, 1.7, 2.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 times higher than that of a control buffer.

In some of the methods described herein, HPV antigens are administered to a subject. In some aspects, these methods include introducing the nucleic acid molecules of the invention into the subject, followed by electroporation. In a specific embodiment, a nucleic acid molecule (e.g., a plasmid encoding an antigen or therapeutic protein of interest) is injected into the target tissue of a subject, such as the skin or muscle, using a conventional needle or a needle-free injection device. Shortly after the injection, a handheld electroporation device is applied to the injection site, such as by contacting the skin or tissue. The device delivers brief, controlled electrical pulses to the tissue, creating transient pores in the cell membranes, making them more permeable to the nucleic acid molecule, which then enters the cells through the pores created by electroporation. Once inside the cells, the nucleic acid molecule is translated into the desired antigen or therapeutic protein. The produced antigen stimulates an immune response, which can protect against the targeted pathogen. In the case of therapeutic proteins, they can exert their intended effects within the cells or tissues.

In some embodiments, the methods may involve administering multiple copies of a single nucleic acid molecule, such as a single plasmid, or multiple copies of two or more different nucleic acid molecules, such as two or more different plasmids. The number of different nucleic acid molecules administered can vary depending on the specific application and may include two, three, four, five, six, seven, eight, nine, ten, or more distinct nucleic acid sequences. This approach allows for the delivery of multiple HPV antigens or the co-delivery of additional immunostimulatory factors to enhance the immune response.

Genetic constructs containing the HPV antigen-encoding nucleic acids can be administered using various methods, including electroporation devices, traditional syringes, standard needles, side port needles (as described in U.S. Publ. No. 2023/0017972, incorporated herein by reference), needleless injection devices, or “microprojectile bombardment gene guns.” Each of these methods has its advantages and can be selected based on factors such as the target tissue, the desired level of gene expression, and the specific application.

Several minimally invasive electroporation devices and methods have been described in the literature and are incorporated herein by reference. These include the devices and methods disclosed in published U.S. Patent Application No. 20080234655; U.S. Pat. Nos. 6,520,950; 7,171,264; 6,208,893; 6,009,347; 6,120,493; 7,245,963; 7,328,064; 6,763,264; and US20240123052. These devices and methods are designed to efficiently deliver nucleic acid molecules into cells while minimizing tissue damage and discomfort to the subject. By using these minimally invasive electroporation techniques, the HPV antigen-encoding nucleic acids can be effectively introduced into the subject's cells, leading to the production of the HPV antigen and the stimulation of an immune response.

### 2. Chemical Methods

Chemical methods for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

### B. Viral-Based Delivery Systems

Also provided herein are viral-based delivery systems, such as viral vectors for delivering nucleic acids. Representative viral vectors include adeno-associated viral vectors, adenoviral vectors, retroviral vectors, and herpes virus-based vectors. Viral vectors may be used as delivery vehicles for nucleic acids encoding a therapeutic molecule, for example, an anti-inflammatory agent, while also avoiding immune-surveillance by host cells. Retrovirus, adenovirus, adeno-associated virus (AAV), and herpes simplex virus have all been adapted for viral vector applications. Robbins et al., Pharmacology & Therapeutics, 80:35-47 (1998). In particular, recombinant adenoviral vectors offer high levels of transgene expression in which the vector remains as episomal DNA without integration into the host genome. The high transduction efficiency and high levels of short-term gene expression make adenoviral vectors ideal for gene therapy and vaccine applications. Furthermore, these viral vectors may be made replication-defective by the deletion of essential viral genes and the replacement thereof with expressions cassettes comprising a foreign therapeutic gene.

Viral vectors that are not infectious in humans or engineered to remove or inactivate their infectious properties are desired for use in genetic therapy as they are efficient at delivering transgenes and can deliver a high payload of nucleic acids to dendritic cells.

The efficacy of treatment using viral vectors, however, is limited by their immunogenicity. For example, human adenoviral vectors are commonly used in genetic therapy; however, as a majority of the U.S. population has been exposed to wild-type forms of such viruses, much of the population has pre-existing immunity thereto. As a result, such vectors and the transgenes carried thereon are quickly cleared from the bloodstream. Further, the immunogenicity of such vectors limit their efficacy in cases of repeat dosing.

### 1. Retroviral Vectors

In certain embodiments, the viral vector is a retroviral vector, such as a lentivirus vector. Vectors derived from retroviruses are suitable tools for achieving long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

### 2. Adeno-Associated Viral Vectors

In certain embodiments, the viral vector is an adeno-associated viral vector. Such vectors are derived from adeno-associated viruses. An advantage to the use of such vectors is that they are low immunogenicity in humans. Another advantage of using such vectors is that they are small and compact and thus can be efficiently used in delivering genes to a cell. However, their size also limits their payload capacity as compared with the use of an adenoviral vector. They are also more difficult to produce as compared with adenoviral vectors. In addition, they have narrow tissue tropism.

### 3. Herpes Virus-Based Vectors

In certain embodiments, the viral vector is a herpes virus-based vector. Such vectors are derived from the herpes simplex virus (HSV). Such vectors are known to be able to infect a wide variety of cell types and have a long persistence in the host. However, they are more difficult to engineer as compared with adenoviral vectors.

### 4. Adenoviral-Based Vectors

In certain embodiments, the viral vector is an adenoviral vector. Such vectors are derived from the adenovirus, for example, a human adenovirus (e.g. human Ad5 type adenovirus), an avian adenovirus, or a gorilla adenovirus. Adenoviruses are generally associated with benign pathologies in humans, and the genomes of adenoviruses isolated from a variety of species, including humans, have been extensively studied. Adenoviral vectors are advantageous as they are capable of infecting a wide variety of cell types. They are also capable of being relatively easily engineered and can carry a high payload.

The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype.

The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. In certain embodiments, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

Adenovirus is a medium-sized (90-100 nm), non-enveloped icosahedral virus containing approximately 36 kb of double-stranded DNA. The adenovirus capsid mediates the key interactions of the early stages of the infection of a cell by the virus, and is required for packaging adenovirus genomes at the end of the adenovirus life cycle. The capsid comprises 252 capsomeres, which includes 240 hexons, 12 penton base proteins, and 12 fibers. Ginsberg et al., Virology, 28: 782-783 (1966). The hexon comprises three identical proteins, namely polypeptide II. Roberts et al., Science, 232: 1148-1151 (1986). The penton base comprises five identical proteins and the fiber comprises three identical proteins. Proteins IIIa, VI, and IX are present in the adenoviral coat and are believed to stabilize the viral capsid. Stewart et al., Cell, 67: 145-54 (1991) and Stewart et al., EMBO J., 12(7): 2589-99 (1993). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications. For example, adenoviruses can be produced in high titers (e.g., about 10¹³ particle units (PU)), and can transfer genetic material to non-replicating and replicating cells. In addition, the adenoviral genome can be manipulated to carry a large amount of exogenous DNA (up to about 8 kb), and the adenoviral capsid can potentiate the transfer of even longer sequences. Curiel et al., Hum. Gene Ther., 3: 147-154 (1992). Additionally, adenoviruses generally do not integrate into the host cell chromosome, but rather are maintained as a linear episome, thereby minimizing the likelihood that a recombinant adenovirus will interfere with normal cell function.

The adenovirus may be modified, for example, using methods known in the art, to be used as an adenoviral vector, e.g., a gene delivery vehicle. The adenovirus and adenoviral vector may be replication-competent, conditionally replication-competent, or replication-deficient.

A replication-competent adenovirus or adenoviral vector can replicate in typical host cells, i.e., cells typically capable of being infected by an adenovirus. A replication-competent adenovirus or adenoviral vector can have one or more mutations as compared to the wild-type adenovirus (e.g., one or more deletions, insertions, and/or substitutions) in the adenoviral genome that do not inhibit viral replication in host cells. For example, the adenovirus or adenoviral vector can have a partial or entire deletion of the adenoviral early region known as the E3 region, which is not essential for propagation of the adenovirus or adenoviral genome.

A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., promoter. In such an embodiment, replication requires the presence or absence of specific factors that interact with the transcription control sequence. Conditionally-replicating adenoviral vectors are further described in U.S. Pat. No. 5,998,205.

A replication-deficient adenovirus or adenoviral vector is an adenovirus or adenoviral vector that requires complementation of one or more gene functions or regions of the adenoviral genome that are required for replication as a result of, for example, a deficiency in one or more replication-essential gene function or regions, such that the adenovirus or adenoviral vector does not replicate in typical host cells, especially those in a human to be infected by the adenovirus or adenoviral vector.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions may be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

Whether the adenovirus or adenoviral vector is replication-competent or replication-deficient, the adenovirus or adenoviral vector typically retains at least a portion of the adenoviral genome. The adenovirus or adenoviral vector can comprise any portion of the adenoviral genome, including protein coding and/or non-protein coding regions. The adenovirus or adenoviral vector may comprise, for example, at least one nucleic acid sequence that encodes an adenovirus protein. The adenovirus or adenoviral vector can comprise a nucleic acid sequence that encodes any suitable adenovirus protein, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions).

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified to decrease the adenoviral vector's ability or inability to be recognized by a neutralizing antibody directed against the wild-type coat protein, as described in International Patent Application WO 98/40509.

In certain embodiments, the adenovirus or adenoviral vector comprises one or more nucleic acid sequences that encode the pIX protein, the DNA polymerase protein, the penton protein, the hexon protein, and/or the fiber protein. The adenovirus or adenoviral vector can comprise a full-length nucleic acid sequence that encodes a full-length amino acid sequence of an adenovirus protein. Alternatively, the adenovirus or adenoviral vector can comprise a portion of a full-length nucleic acid sequence that encodes a portion of a full-length amino acid sequence of an adenovirus protein. A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The adenovirus pIX protein is present in the adenovirus capsid, has been shown to strengthen hexon nonamer interactions, and is essential for the packaging of full-length genomes. See, e.g., Boulanger et al., J Gen. Virol., 44: 783-800 (1979); Horwitz M. S., “Adenoviridae and their replication” in Virology, 2^{nd} ed., B. N. Fields et al. (eds.), Raven Press, Ltd., New York, pp. 1679-1721 (1990), Ghosh-Choudhury et al., EMBO J., 6: 1733-1739 (1987) and van Oostrum et al., J. Virol., 56: 439-448 (1985). In addition to its contribution to adenovirus structure, pIX also has been shown to exhibit transcriptional properties, such as stimulation of adenovirus major late promoter (MLP) activity. See, e.g., Lutz et al., J. Virol., 71(7): 5102-5109 (1997). Nucleic acid sequences that encode all or a portion of an adenovirus pIX protein have been described, for example, in WO 2019/173465 and WO 2022/115470.

The adenovirus DNA polymerase protein is essential for viral DNA replication both in vitro and in vivo. The polymerase co-purifies in a complex with the precursor (pTP) of the terminal protein (TP), which is covalently attached to the 5′ ends of adenovirus DNA. Field et al., J. Biol. Chem., 259: 9487-9495 (1984). Both the adenovirus DNA polymerase and pTP are encoded by the E2 region. The polymerase protein is required for the expression of all the structural proteins except for pIX. Without the gene sequence for polymerase protein, polymerase protein is not produced. As a result, the viral genome is not replicated, the Major Late Promoter is not activated, and the capsid proteins are not expressed. Nucleic acid sequences that encode all or a portion of an adenovirus DNA polymerase protein have been described, for example, in WO 2019/173465 and WO 2022/115470.

The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, hexon is a primary target for modification in order to reduce neutralization of adenoviral vectors. See, e.g., Gall et al., J. Virol., 72: 10260-264 (1998), and Rux et al., J. Virol., 77(17): 9553-9566 (2003). The major structural features of the hexon protein are shared by adenoviruses across serotypes, but the hexon protein differs in size and immunological properties between serotypes. Jornvall et al., J. Biol. Chem., 256(12): 6181-6186 (1981). A comparison of 15 adenovirus hexon proteins revealed that the predominant antigenic and serotype-specific regions of the hexon appear to be in loops 1 and 2 (i.e., LI or 11, and LII or 12, respectively), within which are seven discrete hypervariable regions (HVR1 to HVR7) varying in length and sequence between adenoviral serotypes. Crawford-Miksza et al., J. Virol., 70(3): 1836-1844 (1996). Nucleic acid sequences that encode all or a portion of an adenovirus hexon protein have been described, for example, in WO 2019/173465 and WO 2022/115470.

The adenovirus fiber protein is a homotrimer of the adenoviral polypeptide IV that has three domains: the tail, shaft, and knob. Devaux et al., J. Molec. Biol., 215: 567-88 (1990), Yeh et al., Virus Res., 33: 179-98 (1991). The fiber protein mediates primary viral binding to receptors on the cell surface via the knob and the shaft domains. Henry et al., J. Virol., 68(8): 5239-46 (1994). The amino acid sequences for trimerization are located in the knob, which appears necessary for the amino terminus of the fiber (the tail) to properly associate with the penton base. Novelli et al., Virology, 185: 365-76 (1991). In addition to recognizing cell receptors and binding the penton base, the fiber contributes to serotype identity. Fiber proteins from different adenoviral serotypes differ considerably. See, e.g., Green et al., EMBO J., 2: 1357-65 (1983), Chroboczek et al., Virology, 186: 280-85 (1992), and Signas et al., J. Virol., 53: 672-78 (1985). Thus, the fiber protein has multiple functions key to the life cycle of adenovirus. Nucleic acid sequences that encode all, or a portion of, an adenovirus fiber protein have been described, for example, in WO 2019/173465 and WO 2022/115470.

The adenovirus penton base protein is located at the vertices of the icosahedral capsid and comprises five identical monomers. The penton base protein provides a structure for bridging the hexon proteins on multiple facets of the icosahedral capsid, and provides the essential interface for the fiber protein to be incorporated in the capsid. Each monomer of the penton base contains an RGD tripeptide motif. Neumann et al., Gene, 69: 153-157 (1988). The RGD tripeptide mediates binding to av integrins and adenoviruses that have point mutations in the RGD sequence of the penton base are restricted in their ability to infect cells. Bai et al., J. Virol., 67: 5198-5205 (1993). Thus, the penton base protein is essential for the architecture of the capsid and for maximum efficiency of virus-cell interaction. Nucleic acid sequences that encode all, or a portion of, an adenovirus penton base protein have been described, for example, in WO 2019/173465 and WO 2022/115470.

The adenovirus or adenoviral vector can comprise one, two, three, four, or all five of the aforementioned sequences alone or in any combination. In this respect, the adenovirus or adenoviral vector may comprise any combination of any two of the aforementioned sequences, any combination of any three of the aforementioned sequences, any combination of any four of the aforementioned sequences, or all five of the aforementioned sequences.

In certain embodiments, the adenovirus or adenoviral vector is replication-deficient, such that the replication-deficient adenovirus or adenoviral vector requires complementation of at least one replication-essential gene function of one or more regions of the adenoviral genome for propagation (e.g., to form adenoviral vector particles).

The replication-deficient adenovirus or adenoviral vector can be modified in any suitable manner to cause the deficiencies in the one or more replication-essential gene functions in one or more regions of the adenoviral genome for propagation. The complementation of the deficiencies in the one or more replication-essential gene functions of one or more regions of the adenoviral genome refers to the use of exogenous means to provide the deficient replication-essential gene functions. Such complementation can be effected in any suitable manner, for example, by using complementing cells and/or exogenous DNA (e.g., helper adenovirus) encoding the disrupted replication-essential gene functions.

In some embodiments, the adenovirus or adenoviral vector is deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenoviral vector (HC-Ad). See Morsy et al., Proc. Natl. Acad. Sci. USA, 95: 965-976 (1998); Chen et al., Proc. Natl. Acad. Sci. USA, 94: 1645-1650 (1997); and Kochanek et al., Hum. Gene Ther., 10: 2451-2459 (1999). The adenoviral vector also can have essentially the entire adenoviral genome removed, in which case at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large insert.

In certain embodiments, the adenoviral vector is “multiply-deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Examples of replication-deficient adenoviral vectors are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the E1B subregion of the adenoviral genome for the adenovirus or adenoviral vector to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus or adenoviral vector to propagate (e.g., to form adenoviral vector particles).

The E3 region does not include any replication-essential gene functions, such that a deletion of the E3 region in part or in whole does not require complementation of any gene functions in the E3 region for the adenovirus or adenoviral vector to propagate (e.g., to form adenoviral vector particles). In the context of the present disclosure, the E3 region is defined as the region that initiates with the open reading frame that encodes a protein with high homology to the 12.5K protein from the E3 region of human adenovirus 5 (NCBI reference sequence AP_000218) and ends with the open reading frame that encodes a protein with high homology to the 14.7K protein from the E3 region of human adenovirus 5 (NCBI reference sequence AP_000224.1). The E3 region can be deleted in whole or in part, or retained in whole or in part. The size of the deletion can be tailored so as to retain an adenovirus or adenoviral vector whose genome closely matches the optimum genome packaging size. A larger deletion will accommodate the insertion of larger heterologous nucleic acid sequences in the adenovirus or adenoviral genome. In some embodiments of the present disclosure, the L4 polyadenylation signal sequences, which reside in the E3 region, are retained.

The E4 region comprises multiple open reading frames (ORFs). An adenovirus or adenoviral vector with a deletion of all of the open reading frames of the E4 region except ORF6, and in some cases ORF3, does not require complementation of any gene functions in the E4 region for the adenovirus or adenoviral vector to propagate (e.g., to form adenoviral vector particles). Conversely, an adenovirus or adenoviral vector with a disruption or deletion of ORF6, and in some cases ORF3, of the E4 region (e.g., with a deficiency in a replication-essential gene function based in ORF6 and/or ORF3 of the E4 region), with or without a disruption or deletion of any of the other open reading frames of the E4 region or the native E4 promoter, polyadenylation sequence, and/or the right-side inverted terminal repeat (ITR), requires complementation of the E4 region (specifically, of ORF6 and/or ORF3 of the E4 region) for the adenovirus or adenoviral vector to propagate (e.g., to form adenoviral vector particles).

The late regions of the adenoviral genome include the L1, L2, L3, L4, and L5 regions. The adenovirus or adenoviral vector also can have a mutation in the major late promoter (MLP), as discussed in International Patent Application Publication WO 2000/000628, which can render the adenovirus or adenoviral vector replication-deficient if desired.

The one or more regions of the adenoviral genome that contain one or more deficiencies in replication-essential gene functions desirably are one or more early regions of the adenoviral genome, i.e., the E1, E2, and/or E4 regions. Thus, in certain embodiments, the adenoviral vector lacks all or part of such regions.

The replication-deficient adenovirus or adenoviral vector also can have one or more mutations as compared to the wild-type adenovirus (e.g., one or more deletions, insertions, and/or substitutions) in the adenoviral genome that do not inhibit viral replication in host cells. Thus, in addition to one or more deficiencies in replication-essential gene functions, the adenovirus or adenoviral vector can be deficient in other respects that are not replication-essential. For example, the adenovirus or adenoviral vector can have a partial or entire deletion of the adenoviral early region known as the E3 region, which is not essential for propagation of the adenovirus or adenoviral genome.

In some embodiments, the adenovirus or adenoviral vector is replication-deficient and requires, at most, complementation of the E1 region or the E4 region of the adenoviral genome, for propagation (e.g., to form adenoviral vector particles). In some such embodiments, the adenoviral vector may lack all or a portion of the E1 and/or E4 region. Thus, the replication-deficient adenovirus or adenoviral vector requires complementation of at least one replication-essential gene function of the E1A subregion and/or the E1B region of the adenoviral genome (denoted an E1-deficient adenoviral vector), or the E4 region of the adenoviral genome (denoted an E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, and at least one gene function of the nonessential E3 region of the adenoviral genome (denoted an E1/E3-deficient adenoviral vector). The adenovirus or adenoviral vector can be deficient in at least one replication-essential gene function (desirably all replication-essential gene functions) of the E4 region of the adenoviral genome, and at least one gene function of the nonessential E3 region of the adenoviral genome (denoted an E3/E4-deficient adenoviral vector).

In some embodiments, the adenovirus or adenoviral vector is replication-deficient and requires, at most, complementation of the E2 region, preferably the E2A subregion, of the adenoviral genome, for propagation (e.g., to form adenoviral vector particles). Thus, the replication-deficient adenovirus or adenoviral vector requires complementation of at least one replication-essential gene function of the E2A subregion of the adenoviral genome (denoted an E2A-deficient adenoviral vector) for propagation (e.g., to form adenoviral vector particles). The adenovirus or adenoviral vector can be deficient in at least one replication-essential gene function (desirably all replication-essential gene functions) of the E2A region of the adenoviral genome and at least one gene function of the nonessential E3 region of the adenoviral genome (denoted an E2A/E3-deficient adenoviral vector).

In some embodiments, the adenovirus or adenoviral vector is replication-deficient and requires, at most, complementation of the E1 and E4 regions of the adenoviral genome for propagation (e.g., to form adenoviral vector particles). In some such embodiments, the adenoviral vector may lack all or a portion of the E1 and/or E4 region. Thus, the replication-deficient adenovirus or adenoviral vector requires complementation of at least one replication-essential gene function of both the E1 and E4 regions of the adenoviral genome (denoted an E1/E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, at least one replication-essential gene function of the E4 region of the adenoviral genome, and at least one gene function of the nonessential E3 region of the adenoviral genome (denoted an E1/E3/E4-deficient adenoviral vector). The adenovirus or adenoviral vector preferably requires, at most, complementation of the E1 region of the adenoviral genome for propagation, and does not require complementation of any other deficiency of the adenoviral genome for propagation. More preferably, the adenovirus or adenoviral vector requires, at most, complementation of the E1 and E4 regions of the adenoviral genome for propagation, and does not require complementation of any other deficiency of the adenoviral genome for propagation.

The adenovirus or adenoviral vector, when deficient in multiple replication-essential gene functions of the adenoviral genome (e.g., an E1/E4-deficient adenoviral vector), can include a spacer sequence to provide viral growth in a complementing cell line similar to that achieved by adenoviruses or adenoviral vectors deficient in a single replication-essential gene function (e.g., an E1-deficient adenoviral vector). The spacer sequence can contain any nucleotide sequence or sequences which are of a desired length, such as sequences at least about 15 base pairs (e.g., between about 15 nucleotides and about 12,000 nucleotides), preferably about 100 nucleotides to about 10,000 nucleotides, more preferably about 500 nucleotides to about 8,000 nucleotides, even more preferably about 1,500 nucleotides to about 6,000 nucleotides, and most preferably about 2,000 to about 3,000 nucleotides in length, or a range defined by any two of the foregoing values. The spacer sequence can be coding or non-coding and native or non-native with respect to the adenoviral genome, but does not restore the replication-essential function to the deficient region. The spacer also can contain an expression cassette. More preferably, the spacer comprises a polyadenylation sequence and/or a gene that is non-native with respect to the adenovirus or adenoviral vector. The use of a spacer in an adenoviral vector is further described in, for example, U.S. Pat. No. 5,851,806 and International Patent Application Publication WO 1997/021826.

By removing all or part of the adenoviral genome, for example, the E1, E3, and E4 regions of the adenoviral genome, the resulting adenovirus or adenoviral vector is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of adenovirus or the adenoviral vector particle. The exogenous nucleic acid sequence preferably is positioned in the E1 region, the E3 region, or the E4 region of the adenoviral genome.

The replication-deficient adenovirus or adenoviral vector of the present disclosure can be produced in complementing cell lines that provide gene functions not present in the replication-deficient adenovirus or adenoviral vector, but required for viral propagation, at appropriate levels in order to generate high titers of viral vector stock. Such complementing cell lines are known and include Human Embryonic Kidney (HEK) 293 cells (described in, e.g., Graham et al., J. Gen. Virol., 36: 59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 95/34671 and Brough et al., J. Virol., 71: 9206-9213 (1997)). Other suitable complementing cell lines to produce the replication-deficient adenovirus or adenoviral vector of the present disclosure include complementing cells that have been generated to propagate adenoviral vectors encoding transgenes whose expression inhibits viral growth in host cells (see, e.g., U.S. Patent Application Publication No. 2008/0233650). Additional suitable complementing cells are described in, for example, U.S. Pat. Nos. 6,677,156 and 6,682,929, and International Patent Application Publication WO 2003/020879.

In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the replication-deficient adenovirus or adenoviral vector. Alternatively, the inventive adenovirus or adenoviral vector can comprise a non-native replication-essential gene that complements for the one or more replication-essential gene functions lacking in the inventive replication-deficient adenovirus or adenoviral vector. For example, an E1/E4-deficient adenoviral vector can be engineered to contain a nucleic acid sequence encoding E4 ORF 6 that is obtained or derived from a different adenovirus (e.g., an adenovirus of a different serotype than the inventive adenovirus or adenoviral vector, or an adenovirus of a different species than the inventive adenovirus or adenoviral vector).

### a. Gorilla-Based Adenovirus Vectors

In some embodiments, the adenovirus described herein is isolated from a gorilla. The Western Gorilla species includes the subspecies Western Lowland Gorilla (Gorilla gorilla gorilla) and Cross River Gorilla (Gorilla gorilla diehli) and the Eastern Gorilla species includes the subspecies Mountain Gorilla (Gorilla beringei beringei) and Eastern Lowland Gorilla (Gorilla beringei graueri). See, e.g., Wilson and Reeder, eds., Mammalian Species of the World, 3^{rd} ed., Johns Hopkins University Press, Baltimore, Md. (2005). In some embodiments, the adenovirus of the present disclosure is isolated from Mountain Gorilla (Gorilla beringei beringei). Previous research has characterized numerous gorilla adenoviruses and their genomic sequences (see, e.g., WO 2013/052832, WO 2013/052811, WO 2013/052799; WO 2019/173465, WO 2022/115470).

Gorilla adenoviruses share similarities with human adenoviruses in terms of vector design and safety, offering benefits like efficient transgene delivery and replication incompetence through targeted deletions. Importantly, compared to human adenoviruses, pre-existing human immunity to gorilla adenoviruses is minimal. This lack of recognition by human immune systems minimizes potential pre-existing immunity hurdles in gene therapy and vaccine applications.

In certain embodiments, the adenoviral vector is derived from a gorilla adenovirus type 40 (GAd40), such as GC44, GC45, or GC46. In certain embodiments, the adenoviral vector represents a functional adaptation of the aforementioned. These adaptations may encompass sequences encoding functional variants of their constituent components, such as the E2B, E2A, E3, and L1-L5 regions, as well as inverted terminal repeats. Also envisaged are functional adaptations of such vectors featuring codon degenerate variants of the sequences encoding the E2B, E2A, E3, and L1-L5 regions.

In particularly preferred embodiments, the adenoviral vector is derived from GC46, a newly-isolated and unique gorilla adenovirus strain, isolated from a healthy African gorilla stool specimen. This adenovirus is closely related to and clusters phylogenetically with the human species C adenoviruses based on hexon, DNA polymerase and Exon 4 ORF6 protein sequence comparison. Duncan et al., Virology, 444:119-123 (2013). The sero-prevalence of gorilla adenovirus type GC46, is less than about 6% in the United States. In comparison, the sero-prevalence of Ad5 type is about 57%, with most of the seropositive individuals having high titers (above 200 IC90). Johnson et al., Molecular Therapy, 22:196-205 (2014). Therefore, compared to traditional adenovirus therapies based on the Ad5 serotype, pre-existing neutralizing activity to gorilla adenovirus type GC46 is rare and weak in the United States. In addition, comparative studies from human sera samples from Sub-Saharan Africa confirmed the rare and weak pre-existing neutralizing activity in the human population. These data suggest that pre-existing neutralizing activity to GC46 will not significantly interfere with molecular vaccines and therapeutics built on this platform, which makes the gorilla adenovirus GC46 well suited as a back bone viral vector.

In preferred embodiments of the present invention, the gorilla adenovirus vaccine encodes a fusion of selected regions of HPV proteins that are expressed in HPV-6 and HPV-11 infected cells (e.g., HPV-E2, HPV-E4, HPV-E6 and HPV-E7).

In a specific embodiment of the present invention, the gorilla adenovirus vaccine encodes 791 amino acids of HPV protein, of which 731 amino acids (92.4%) are derived from HPV-6 and 60 amino acids are derived (7.6%) from HPV-11.

In certain embodiments, the adenovirus vector is a gorilla adenovirus vector engineered to delete portions of or the entire E1 and/or E4 regions. The deletion in the E1 region may, for example, render the adenovirus vector replication-deficient and include bases 459 through 3411, resulting in deletion of the E1A and E1B promoters and open reading frames. The deletion in the E4 region may, for example, be inclusive of bases 34144 to 36824 and remove all the E4 open reading frames (ORFs), therefore eliminating essential elements for gorilla adenovirus replication. (The gorilla adenovirus coordinates provided herein are based on a wild-type adenovirus genome size of 37,213 base pairs.)

The modified gorilla adenovirus vector with the deletions of and/or in the E1 and/or E4 regions may provide one or more advantages over unmodified vector backbones. For example, the extended deletions of the adenoviral genome may provide enhanced payload capacity to the adenovirus vector. A second potential advantage is reduced risk of Replication Competent Adenovirus (RCA) generation during adenovirus vector production. A third advantage is that the elimination of E1 and E4 expression products may work to further silence other regions of the viral genome.

Thus, in one aspect, the gorilla adenovirus vectors described herein have the E1 region, or portions thereof, deleted. In another aspect, the gorilla adenovirus vectors described herein have the E4 region, or portions thereof, deleted. In another aspect the gorilla adenoviral vectors described herein have both the E1 and E4 regions, or portions thereof, deleted. In one aspect, the deletion(s) in the E1 and/or E4 regions comprise from about 100 to about 5,000 base pairs (bp) in length as compared to the wild-type. For example, the deletion(s) in the E1 and/or E4 regions may comprise about 100 bp, about 500 bp, about 1,000 bp, about 1,500 bp, about 2,000 bp, about 2,500 bp, about 3,000 bp, about 3,500 bp, about 4,000 bp, about 4,500 bp, or about 5,000 bp in length as compared to the wild-type. In some embodiments, the deletion(s) in the E1 and/or E4 regions comprise from about 100 bp to about 5,000 bp, or about 500 bp to about 4,500 bp, about 750 bp to about 4,000 bp, or about 1,000 bp to about 3,750 bp, or about 1,250 bp to about 3,500 bp, or about 1,500 bp to about 3,500 bp, or about 1,750 bp to about 3,500 bp, or about 2,000 bp to about 3,500 bp, or about 2,000 bp to about 3,000 bp. In specific embodiments, the deletion(s) in the E1 and/or E4 regions comprise about 3,000 bp in length as compared to the wild-type.

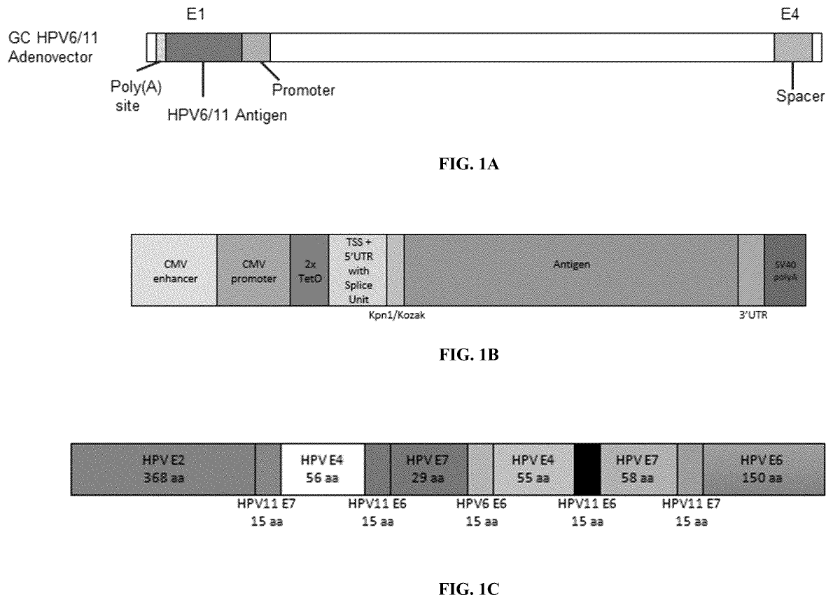

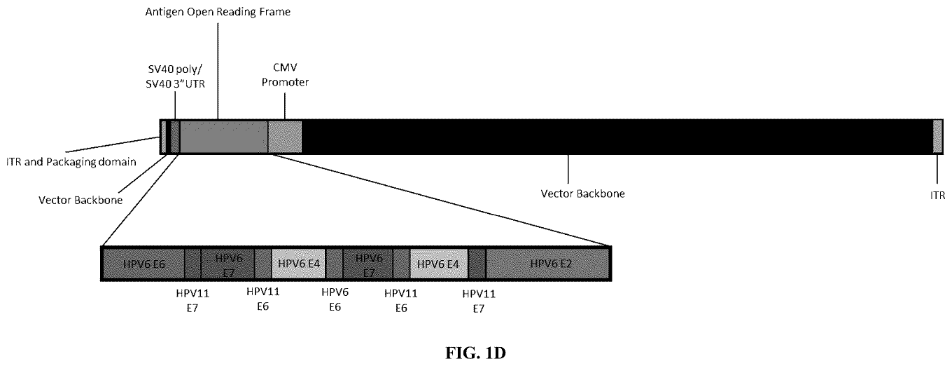

In certain embodiments, deletion of the E4 region removes all predicted open reading frames (ORFs) therein. To avoid the potential for low levels of production of adenoviral vectors with E4 deletions, spacer sequences may be inserted within the E4 deleted region, as depicted in FIG. 1, to stop any potential transcription initiated from the retained E4 promoter. In one aspect, the gorilla adenoviral vector described herein comprises a spacer sequence inserted in place of the deleted portion of the E4 region. In one aspect, the spacer sequence comprises a Bovine Growth Hormone polyadenylation (BGH polyA) signal sequence that is inserted in place of the deleted E4 ORFs, but any suitable spacer sequence may be used. In some aspects, the spacer sequence is about 10 to about 500 base pairs (bp) in length. For example, the spacer sequence may be about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 bp in length. Alternatively, the spacer sequence may be about 50 bp to about 100 bp, about 100 bp to about 150 bp, about 150 bp to about 200 bp, about 200 bp to about 250 bp, about 250 bp to about 300 bp, about 300 bp to about 350 bp, about 350 bp to about 400 bp, about 400 bp to about 450 bp, or about 450 bp to about 500 bp, as compared to the wild-type. The spacer may also be any length within these ranges. For instance, the spacer may be about 250 bp to about 350 bp, about 260 bp to about 340 bp, about 270 bp to about 330 bp, about 280 bp to about 320 bp, or about 290 bp to about 310 bp in length. In one aspect, the spacer sequence is about 300 base pairs in length. In yet another aspect, the spacer sequence is 278 bp.

In another aspect, the location of the spacer sequence is at about 34,700 thru 35,000 base pairs of the vector genome as compared to the wild-type. In yet another aspect, the location of the spacer sequence is at 34,692 thru 34,969 base pairs of the vector genome as compared to the wild-type. In another aspect the spacer sequence comprises a nucleic acid sequence of SEQ ID NO: 95.

In certain embodiments, the vector has all of the E1 and E4 regions deleted. In some such embodiments, the E1 or E4 region is replaced with an expression cassette comprising a transgene or a spacer. In some such embodiments, the E1 region is replaced with the expression cassette and the E4 region is replaced with a spacer.

In one embodiment, an E1/E4-deficient GC46 adenoviral vector can be produced in any complementing cell line that provides for the functions of E1 and E4 ORF6, for example, an engineered 293 cell. Such cells may be cultured, for example, in a serum-free suspension in a shaker flask and infected with master virus bank at a multiplicity of infection (MOI) of 100 PU/cell. The culture harvest may be downstream processed and purified using three rounds of cesium chloride density gradient ultracentrifugation to yield a highly purified material. This material may then be frozen and later thawed and sterile-filtered and filled into vials which can be stored in a freezer at about −60 to about −90° C.

In some embodiments, the vector of the present invention may be made by isolating the GC46 gorilla adenovector from nonhuman primate sources, cloning the isolated GC46 genome, deleting the E1 and E4 regions of GC46, and inserting an expression cassette in the E1 region that expresses a human papilloma virus (HPV) 6/11 antigen design and is under the control of a cytomegalovirus (CMV) immediate early promoter. In one embodiment described below, the CMV-HPV 6/11 antigen design contains epitopes of HPV 6 and 11-namely, key immunogenic peptides from E2 (HPV6), E4 (HPV6), E6 (HPV6/11), and E7 (HPV6/11) where the HPV6-derived peptides have high sequence similarity with HPV11.

In some embodiments, the vector of the present invention encodes any of the HPV antigen regions or variants thereof described herein. For example, a vector can comprise a nucleic acid sequence having at least 80% identity to SEQ ID NO: 68.

### C. Non-Viral Based Delivery Systems

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally-occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −200 C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

In some instances, polynucleotides encoding polypeptides can also be introduced into cells using non-viral based delivery systems, such as the “Sleeping Beauty (SB) Transposon System.” In embodiments, a modified effector cell described herein and other genetic elements are delivered to a cell using the SB11 transposon system, the SB100X transposon system, the SB110 transposon system, the piggyBac transposon system (see, e.g., Wilson et al., “PiggyBac Transposon-mediated Gene Transfer in Human Cells,” Molecular Therapy 15:139-145 (2007), incorporated herein by reference in its entirety) and/or the piggyBac transposon system (see, e.g., Mitra et al., “Functional characterization of piggyBac from the bat Myotis lucifugus unveils an active mammalian DNA transposon,” Proc. Natl. Acad. Sci USA 110:234-239 (2013). Additional transposases and transposon systems are provided in U.S. Pat. Nos. 6,489,458; 6,613,752, 7,148,203; 7,985,739; 8,227,432; 9,228,180; U.S. Patent Pub. No. 2011/0117072; Mates et al., Nat Genet, 41(6): 753-61 (2009). doi: 10.1038/ng.343. Epub 2009 May 3, Gene Ther., 18(9):849-56 (2011). doi: 10.1038/gt.2011.40. Epub 2011 Mar. 31 and in Ivies et al., Cell, 91(4):501-10, (1997), each of which is incorporated herein by reference in their entirety.

Additional suitable non-viral systems can include integrating expression vectors, which can randomly integrate into the host cell's DNA, or can include a recombination site to enable the specific recombination between the expression vector and the host cell's chromosome. Targeted integration of transgenes into predefined genetic loci is a desirable goal for many applications. First, a first recombination site for a site-specific recombinase is inserted at a genomic site, either at a random or at a predetermined location. Subsequently, the cells are transfected with a plasmid carrying the gene or DNA of interest and the second recombination site and a source for recombinase (expression plasmid, RNA, protein, or virus-expressing recombinase). Recombination between the first and second recombination sites leads to integration of plasmid DNA. Such integrating expression vectors can utilize the endogenous expression control sequences of the host cell's chromosomes to effect expression of the desired protein.

In some embodiments, targeted integration is promoted by the presence of sequences on the donor polynucleotide that are homologous to sequences flanking the integration site. For example, targeted integration using the donor polynucleotides described herein can be achieved following conventional transfection techniques, e.g. techniques used to create gene knockouts or knockins by homologous recombination. In other embodiments, targeted integration is promoted both by the presence of sequences on the donor polynucleotide that are homologous to sequences flanking the integration site, and by contacting the cells with donor polynucleotide in the presence of a site-specific recombinase. By a site-specific recombinase, or simply a recombinase, it is meant is a polypeptide that catalyzes conservative site-specific recombination between its compatible recombination sites. As used herein, a site-specific recombinase includes native polypeptides as well as derivatives, variants and/or fragments that retain activity, and native polynucleotides, derivatives, variants, and/or fragments that encode a recombinase that retains activity.

Also provided herein is a system for integrating heterologous genes in a host cell, said system comprising one or more gene expression cassettes. In some instances, the system includes a first gene expression cassette comprising a first polynucleotide encoding a first polypeptide construct. In other instances, the system can include a second gene expression cassette comprising a second polynucleotide encoding a second polypeptide construct. In yet other instances, the system can include a third gene expression cassette. In one embodiment, one of the gene expression cassettes can comprise a gene switch polynucleotide encoding one or more of: (i) a transactivation domain; (ii) nuclear receptor ligand binding domain; (iii) a DNA-binding domain; and (iv) ecdysone receptor binding domain. In another embodiment, the system further includes recombinant attachment sites; and a serine recombinase; such that upon contacting said host cell with at least said first gene expression cassette, in the presence of said serine recombinase, said heterologous genes are integrated in said host cell.

In some instances, the system further comprises a ligand; such that upon contacting said host cell, in the presence of said ligand, said heterologous gene are expressed in said host cell. In one instance, the system also includes recombinant attachment sites. In some instances, one recombination attachment site is a phage genomic recombination attachment site (attP) or a bacterial genomic recombination attachment site (attB). In one instance, the host cell is an eukaryotic cell. In another instance, the host cell is a human cell. In further instances, the host cell is a T cell or NK cell.

### II. Expression Cassettes

### A. Transgenes

The vector of the present invention may comprise an expression cassette for expressing a transgene. A “transgene” comprises a non-native nucleic acid sequence that is operably linked to appropriate regulatory elements (e.g., a promoter), such that the non-native nucleic acid sequence can be expressed to produce a protein (e.g., peptide or polypeptide). The regulatory elements (e.g., promoter) can be native or non-native to the adenovirus or adenoviral vector.

A “non-native” nucleic acid sequence is any nucleic acid sequence (e.g., DNA, RNA, or cDNA sequence) that is not a naturally occurring nucleic acid sequence of an adenovirus in a naturally occurring position. Thus, the non-native nucleic acid sequence can be naturally found in an adenovirus, but located at a non-native position within the adenoviral genome and/or operably linked to a non-native promoter. The terms “non-native nucleic acid sequence,” “heterologous nucleic acid sequence,” and “exogenous nucleic acid sequence” are synonymous and can be used interchangeably in the context of the present disclosure. The non-native nucleic acid sequence preferably is DNA and preferably encodes a protein (i.e., one or more nucleic acid sequences encoding one or more proteins).

The non-native nucleic acid sequence can encode a therapeutic protein that can be used to prophylactically or therapeutically treat a mammal for a disease. Examples of suitable therapeutic proteins include anti-inflammatory agents such as cytokines, toxins, tumor suppressor proteins, growth factors, hormones, receptors, mitogens, immunoglobulins, neuropeptides, neurotransmitters, and enzymes. Alternatively, the non-native nucleic acid sequence can encode an antigen of a pathogen (e.g., a bacterium or a virus), and the adenovirus or adenoviral vector can be used as a vaccine.

### B. Promoters

Genetic regulatory components of the therapeutic expression cassette are selected to confer a high level expression of the transgene. Thus, another aspect of the present disclosure is an expression cassette that further comprises a promoter. A promoter is a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example, by Northern blot analysis or indirectly by determining the amount of product coded for by the linked nucleic acid sequence, such as a reporter nucleic acid sequence linked to the promoter.

### 1. Inducible Promoters

In certain embodiments, the promoter is an inducible promoter. An inducible promoter is a promoter that is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. In some embodiments, the inducible promoter is part of a genetic switch. The inducible promoter can be a gene switch ligand inducible promoter. In some cases, an inducible promoter can be a small molecule ligand-inducible two polypeptide ecdysone receptor-based gene switch, such as RHEOSWITCH® gene switch, such as the system described in WO 2018/132494. Additional examples of gene switch systems include, without limitation, the systems described in U.S. Pat. Nos. 6,258,603, 7,045,315, U.S. Published Patent Application Nos. 2006/001471 1, 2007/0161086, and International Published Application No. WO 01/70816.

In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919).

An inducible promoter typically utilizes a ligand for dose-regulated control of expression of said at least two genes. In some cases, the ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch

### 2. Non-Inducible Promoters

In certain embodiments, the promoter is a non-inducible promoter, including, e.g., tissue-specific, strong constitutive, or minimal promoters known in the art. Suitable non-inducible promoters may include, for example, a CMV promoter, a SV40 promoter, a CAG promoter, or others. In certain embodiments, the promoter is a CMV promoter.

### 3. Tissue-Specific Promoters

In certain embodiments, the promoter may be a tissue-specific promoter. Herein “tissue-specific” refers to regulated expression of a gene in a subset of tissues or cell types. In some cases, the tissue-specific promoter can be regulated spatially such that the promoter drives expression only in certain tissues or cell types of an organism. In other cases, the tissue-specific promoter can be regulated temporally such that the promoter drives expression in a cell type or tissue differently across time, including during development of an organism. In some cases, the tissue-specific promoter is regulated both spatially and temporally. In certain embodiments, the tissue-specific promoter is activated in certain cell types either constitutively or intermittently at particular times or stages of the cell type. For example, the tissue-specific promoter can be a promoter that is activated when a specific cell such as a T cell or a NK cell is activated. T cells can be activated in a variety of ways, for example, when presented with peptide antigens by MHC class II molecules.

### 4. Synthetic and Engineered Promoters

Synthetic promoters are also contemplated for use in the expression cassette described herein, and may be engineered to improve expression characteristics. Synthetic promoters may include a variety of sub-components including, but not limited to, blocking sequences, enhancers, and various responsive elements.

In certain embodiments, the promoter is an engineered promoter or variant thereof. As described herein, the promoter can incorporate minimal promoter sequences from IL-2 and one or more of the following: nuclear factor of activated T-cells (NFAT) response element(s); NFIL2D response element, NF-κB/TCF response element, NFAT/NFIL2B response element or NFIL2A/OCT response element. NFAT transcription factors are key modulators of effector T-cell states. NFATs are early transcriptional checkpoint progressively driving exhaustion. NFATs are quickly activated in T cells following TCR stimulation and form a protein complex with AP-1 induced by appropriate co-stimulation signaling and regulate effector genes and T-cell functions. NFAT response element(s) can be fused with other minimal promoter sequences (e.g. IL2 minimal promoter) to drive expression of transgenes in response to T cell activation. Further examples of response elements are described in Mattila et al., EMBO J., 9(13):4425-33 (1990).

### 5. Activation-Specific Promoters

In certain embodiments, the promoter is an activation-specific promoter, for example, interleukin-2 (IL2) promoter and Programmed Death (PD)-1 (CD279) promoter. Gene switch components can also be conditionally expressed upon immune cell activation by fusing binding sites for other nuclear factors like NF-κB of proinflammatory signaling pathway to minimal promoter sequence (e.g. IL2).

In certain embodiments, the promoter comprises IL-2 core promoter. In some embodiments, at least one promoter comprises IL-2 minimal promoter. In another embodiment, at least one promoter comprises IL-2 enhancer and promoter variant. In yet another embodiment, at least one promoter comprises NF-κB binding site. In some embodiments, at least one promoter comprises (NF-κB)₁-IL2 promoter variant. In some embodiments, at least one promoter comprises (NF-κB)₃-IL2 promoter variant. In some embodiments, at least one promoter comprises (NF-κB)₆-IL2 promoter variant. In some embodiments, at least one promoter comprises 1× nuclear factor of activated T-cells (NFAT) response elements-IL2 promoter variant. In another embodiment, at least one promoter comprises 3× NFAT response element. In yet another embodiment, at least one promoter comprises 6× NFAT response elements-IL2 promoter variant. In some embodiments, at least one promoter comprises human EF1A1 promoter variant. In some embodiment, at least one promoter comprises human EF1A1 promoter and enhancer. In some embodiments, at least one promoter comprises human UBC promoter. In some embodiments, at least one promoter comprises 6 site GAL4-inducible proximal factor binding element (PFB). In some embodiment, at least one promoter comprises synthetic minimal promoter 1 (inducible promoter). Sequences for such promoters are described in, for example, for example, in WO 2019/173465 and WO 2022/115470.

In certain embodiments, the promoter can be any one or more of: IL-2 core promoter, IL-2 minimal promoter, IL-2 enhancer and promoter variant, (NF-κB)₁-IL2 promoter variant, (NF-κB)₃-IL2 promoter variant, (NF-κB)₆-IL2 promoter variant, 1× NFAT response elements-IL2 promoter variant, 3× NFAT response elements-IL2 promoter variant, 6× NFAT response elements-IL2 promoter variant, human EEF1A1 promoter variant, human EEF1A1 promoter and enhancer, human UBC promoter and synthetic minimal promoter 1.

### 6. Constitutive Promoters

In certain embodiments, the promoter is a constitutive promoter. Examples of such promoters include the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter.

### 7. Viral Promoters

Exemplary promoters used in the vectors described herein include viral promoters in operable combination with a heterologous nucleic acid sequence encoding the cytokine. Exemplary viral promoters may be derived from multiple known viruses, including but not limited to retroviral vectors, lentiviral vectors, adenovirus vectors, adeno-associated virus vectors (AAV), alphavirus vectors and the like. Non-limiting examples of potentially useful viral vectors include Human Immunodeficiency Virus (HIV), Respiratory Syncytial Virus (RSV), Cytomegalovirus (CMV), Simian virus 40, Herpes Simplex Virus (HSV), Adenovirus (AV), Adeno-Associated Virus (AAV), or Lentivirus (LV). For example, specific viral promoters contemplated herein include cytomegalovirus (CMV) immediate early promoter, CAG promoter (which is a combination of the CMV early enhancer element and chicken beta-actin promoter), simian virus 40 (SV40) promoter, the 35S RNA and 19S RNA promoters of cauliflower mosaic virus (CaMV), the coat protein promoter to tobacco mosaic virus (TMV), and any variants thereof. Examples of mammalian promoters include human elongation factor 1α-subunit (EF1-1α) promoter, human ubiquitin C (UCB) promoter, murine phosphoglycerate kinase-1 (PGK) promoter, and any variants thereof.

### 8. Other Promoter Elements

Additional promoter elements, e.g., enhancers (e.g., a promoter enhancer), regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Synthetic promoters useful in the present invention may include enhancer sequences. In one aspect, the enhancer may be an mCMV enhancer sequence. In another aspect, the mCMV enhancer sequence is about 500 to about 1,000 bp in length. In another aspect, the enhancer sequence is about 700 bp in length. In yet another aspect, the enhancer sequence comprises a nucleic acid sequence of SEQ ID NO: 96 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 96, or a conservatively-substituted variant of SEQ ID NO: 96, or a non-conservatively-substituted variant of SEQ ID NO: 96.

In another aspect, the mCMV enhancer comprises transcription factor binding sites. In yet another aspect, the transcription factor binding sites comprise SpI, Ebox, ETS, TRE, CREB, and GATA binding sites.

Responsive elements may also be included in the promoters described herein. Various responsive elements are known in the art. For example, to reduce expression of the transgene during adenovirus production, which can sometimes negatively impact overall production titer, a Tetracyline Responsive Element (TRE, 2× TetO) may be positioned between the TATA box and the transcription initiation site within a promoter element of the promoter. Thus, when the vector is produced in cell lines that express the Tetracycline (Tet) repressor, expression of the transgene driven by a promoter containing a TRE is reduced. Gall et al., Molecular Biotechnology, 35:263-273 (2007). In the absence of tetracycline, the Tet repressor interacts with the TRE element and blocks the initiation of transcription. Upon infection of producer cells that do not express the Tet repressor, normal expression levels are observed. Thus, in one aspect described herein, the synthetic promoter comprises a TRE. The TRE comprises about 10 to about 100 bp in length. In another aspect, the TRE ranges from 10 bp to 100 bp in length. In one aspect, the TRE comprises about 50 bp in length. In yet another aspect, the TRE comprises a nucleic acid sequence of SEQ ID NO: 98 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 98, or a conservatively-substituted variant of SEQ ID NO: 98, or a non-conservatively-substituted variant of SEQ ID NO: 98.

In certain embodiments, the promoter comprises a transcription blocker, an enhancer sequence and a responsive element. In some such embodiments, the promoter comprises an mCMV enhancer and a TRE.

### C. Untranslated Regions

In some embodiments, the expression cassette may include an untranslated region (UTR) to regulate or enhance transgene expression. In one aspect, the expression cassette may include an artificial untranslated region. A 5′UTR with a splice unit has been demonstrated to enhance expression of a transgene cassette. Thus, in an embodiment, the cassette comprises a 5′UTR with a splice unit. In certain embodiments, the 5′UTR is engineered to include a synthetic splice site sequence spanning a canine ATP2A2 intron 2 followed by the 5′ UTR of bovine CSN2 gene. In another aspect, the 5′ UTR comprises a nucleic acid sequence of SEQ ID NO: 99 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 99, or a conservatively-substituted variant of SEQ ID NO: 99, or a non-conservatively-substituted variant of SEQ ID NO: 99.

### D. Termination Sequences

In another aspect, the expression cassette further comprises a termination sequence. The open reading frame of the cytokine transgene may be followed by a termination sequence. Like the promoter sequence, the termination sequence may also include various regulatory elements to ensure proper 3′ transcript end processing. In one aspect, the termination sequence comprises a partial human growth hormone (HGH) 3′ untranslated region. In another aspect, the termination sequence comprises a polyadenylation signal, including but not limited to a SV40 polyadenylation signal and/or a LTR polyadenylation signal. In yet another aspect, the termination sequence comprises a human beta actin (ACTb) transcriptional termination signal sequence. In another aspect, the termination sequence comprises a HGH 3′ untranslated region, a polyadenylation signal, and a human beta actin transcriptional termination sequence. In another aspect, the termination sequence comprises the nucleic acid sequence of SEQ ID NO: 104 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 104, or a conservatively-substituted variant of SEQ ID NO: 104, or a non-conservatively-substituted variant of SEQ ID NO: 104.

### E. Polynucleotide Linkers

Also contemplated herein are expression cassettes and constructs comprising a polynucleotide linker to facilitate the expression of the polynucleotides and functionality of the polypeptides described herein.

In some cases, the linker may be a cleavable linker. The polynucleotide linker can be an oligomer. The polynucleotide linker can be a DNA double strand, single strand, or a combination thereof. In some cases, the linker can be RNA. A polynucleotide linker can be a double-stranded segment of DNA containing desired restriction sites that can be added to create end structures that are compatible with a vector comprising a polynucleotide described herein.

In some cases, a polynucleotide linker can be useful for modifying vectors comprising polynucleotides described herein. For example, a vector modification comprising a polynucleotide linker can be a change in a multiple cloning site, or the addition of a poly-histidine tail. Polynucleotide linkers can also be used to adapt the ends of blunt insert DNA for cloning into a vector cleaved with a restriction enzyme with cohesive end termini. The use of polynucleotide linkers can be more efficient than a blunt ligation into a vector and can provide a method of releasing an insert from a vector in downstream applications. The insert may be a polynucleotide sequence encoding polypeptides useful for therapeutic applications.

In some embodiments, the polynucleotide linker may be ligated into a vector comprising a polynucleotide described herein by a T4 ligase in some cases. To facilitate a ligation an excess of polynucleotide linkers can be added to a composition comprising an insert and a vector. In some cases, an insert and vector are pre-treated before a linker is introduced. For example, pre-treatment with a methylase can prevent unwanted cleavage of insert DNA.

In some cases, the polynucleotides or genes described herein may be separated by 1, 2, 3, 4, 5, 6, 7, 8, 9, or up to 10 linkers.

### 1. IRES Sequences

In some embodiments, the polynucleotide(s) described herein may be linked by an “internal ribosome entry site,” or “IRES” element. An IRES can allow simultaneous expression of multiple genes. For example, an IRES sequence can permit production of multiple proteins from a single mRNA transcript. A ribosome can bind to an IRES in a 5′-cap independent manner and initiate translation.

In an expression cassette comprising an IRES sequence, a first gene can be translated by a cap-dependent, ribosome scanning, mechanism with its own 5′-UTR, whereas translation of a subsequent gene can be accomplished by direct recruitment of a ribosome to an IRES in a cap-independent manner. An IRES sequence can allow eukaryotic ribosomes to bind and begin translation without binding to a 5′ capped end. An IRES sequence can allow expression of multiple genes from one transcript (Mountford and Smith, Trends Genet. 11(5):179-84 (1995)).

In certain cases, an IRES region can be derived from a virus, such as picornavirus, encephalomyocarditis virus, hepatitis C virus IRES sequence. In other cases, an IRES sequence can be derived from an encephalomyocarditis virus. The term “EMCV” or “encephalomyocarditis virus” as used herein, refers to any member isolate or strain of the encephalomyocarditis virus species of the genus of the family Picornaviridae. Examples are: EMCV-R (Rueckert) strain virus and Columbia-SK virus. In some cases, a cellular IRES element, such as eukaryotic initiation factor 4G, immunoglobulin heavy chain binding protein, c-myc proto-oncogene, vascular endothelial growth factor, fibroblast growth factor-I IRES, or any combination or modification thereof can be used. In some cases, a cellular IRES can have increased gene expression when compared to a viral IRES.

An IRES sequence of viral, cellular, or a combination thereof can be utilized in the vector. An IRES can be from encephalomyocarditis (EMCV) or poliovirus (PV). In some cases, an IRES element is selected from a group consisting of Poliovirus (PV), Encephalomyelitis virus (EMCV), Foot-and-mouth disease virus (FMDV), Porcine teschovirus-1 (PTV-1), Aichivirus (AiV), Seneca Valley virus (SVV), Hepatitis C virus (HCV), Classical swine fever virus (CSFV), Human immunodeficiency virus-2 (HIV-2), Human immunodeficiency virus-I (HIV-I), Moloney murine leukemia virus (MoMLV), Feline immunodeficiency virus (FIV), Mouse mammary tumor virus (MMTV), Human cytomegalovirus latency (pUL138), Epstein-Barr virus (EBNA-1), Herpes virus Marek's disease (MDV RLORF9), SV40 polycistronic 19S (SV40 19S), Rhopalosiphum padi virus (RhPV), Cricket paralysis virus (CrPV), Ectropis obliqua picorna-like virus (EoPV), Plautia stali intestine virus (PSIV), Triatoma virus (TrV), Bee paralysis dicistrovirus (IAPV, KBV), Black currant reversion virus (BRV), Pelargonium flower break virus (PFBV), Hibiscus chlorotic ringspot virus (HCRSV), Crucifer-infecting tobamovirus (CrTMV), Potato leaf roll polerovirus (PLRV), Tobacco etch virus (TEV), Giardiavirus (GLV), Leishmania RNA virus-I (LRV-1), and combinations or modifications thereof.

In some cases, an IRES is selected from a group consisting of Apaf-1, XIAP, HIAP2/c-IAP1, DAP5, Bel-2, c-myc, CAT-I, INR, Differentiation LEF-1, PDGF2, HIF-1a, VEGF, FGF2, BiP, BAG-I, CIRP, p53, SHMTI, PITSLREp58, CDKI, Rpr, hid, hsp70, grim, skl, Antennapedia, dFoxO, dinR, Adh-Adhr, HSPIOI, ADH, URE-2, GPRI, NCE102, YMR181a, MSNI, BOil, FLO8, GICI, and any combination or modification thereof. When an IRES element is included between two open reading frames (ORFs), initiation of translation can occur by a canonical 5′-m7GpppN cap-dependent mechanism in a first ORF and a cap-independent mechanism in a second ORF downstream of the IRES element.

In some cases, an IRES sequence can be from about 9 to about 1,000 base pairs. For example, an IRES sequence can be from about 9 to about 150 base pairs, or from about 150 to about 400 base pairs, from about 400 to about 600 base pairs, or from about 600 to 1,000 base pairs. In some embodiments, the IRES sequence is about 9, about 25, about 50, about 75, about 100, about 125, about 150, about 175, about 200, about 225, about 250, about 275, about 300, about 325, about 350, about 275, about 400, about 425, about 450, about 475, about 500, about 525, about 550, about 575, about 600, about 625, about 650, about 675, about 700, about 750, about 800, about 850, about 900, about 950, or about 1,000 base pairs.

In some cases, expression of a downstream gene within a vector comprising an IRES sequence can be reduced. For example, a gene following an IRES sequence can have reduced expression over a gene preceding an IRES sequence. Reduced expression can be from 1% to 99.9% reduction over a preceding gene, including, e.g., a 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, or 99.9% reduction over a preceding gene.

### 2. Viral 2A Sequences

In some embodiments, the polynucleotide(s) described herein may be linked by a viral 2A element or sequence. 2A elements can be shorter than IRES, having from 5 to 100 base pairs. In some cases, a 2A sequence may comprise 5, 10, 15, 20, 25, 30, 35, 40, 45, 50, 55, 60, 65, 70, 75, 80, 85, 90, or 100 base pairs. 2A linked genes can be expressed in one single open reading frame and “self-cleavage” can occur co-translationally between the last two amino acids, GP, at the C-terminus of the 2A polypeptide, giving rise to equal amounts of co-expressed proteins.

A viral 2A sequence can be about 20 amino acids. In some cases, a viral 2A sequence can contain a consensus motif Asp-Val/Ile-Glu-X-Asn-Pro-Gly-Pro (SEQ ID NO: 122). A consensus motif sequence can act co-translationally. For example, formation of a normal peptide bond between a glycine and praline residue can be prevented, which can result in ribosomal skipping and cleavage of a nascent polypeptide. This effect can produce multiple genes at equimolar levels.

A 2A peptide can allow translation of multiple proteins in a single open reading frame into a polypeptide that can be subsequently cleaved into individual polypeptide through a ribosome-skipping mechanism (Funston et al., J Gen. Viral. 89(Pt 2):389-96 (2008)). In some embodiments, a 2A sequence can include: F/T2A, T2A, p2A, 2A, T2A, E2A, F2A, and BmCPV2A, BmIFV2A, and any combination thereof.

In some cases, a vector can comprise an IRES sequence and a 2A linker sequence. In other cases, expression of multiple genes linked with 2A peptides can be facilitated by a spacer sequence (GSG) ahead of the 2A peptides. In some cases, constructs can combine a spacers, linkers, adaptors, promoters, or combinations thereof. For example, a linker can have a spacer (SGSG (SEQ ID NO: 121) or GSG or Whitlow linker) and furin linker (R-A-K—R (SEQ ID NO: 123)) cleavage site with different 2A peptides. A spacer can be an I-Ceui. In some cases, a linker can be engineered. For example, a linker can be designed to comprise chemical characteristics such as hydrophobicity. In some cases, at least two linker sequences can produce the same protein. In other cases, multiple linkers can be used in a vector. For example, genes of interest can be separated by at least two linkers.

### 3. Polypeptide Linkers Encoded by the Polynucleotide(s)

In certain embodiments, the polynucleotides described herein may encode two or more polypeptides. In some of those embodiments, the polynucleotides may be separated by an intervening sequence encoding an intervening linker polypeptide. As used herein, the term “intervening linker polypeptide” means an amino acid sequence separating two or more polypeptides encoded by a polynucleotide, and is distinguishable from the term “peptide linker” which refers to the sequence of amino acids, which is optionally included in a polypeptide construct disclosed herein, to connect the transmembrane domain to the cell surface polypeptide (e.g., comprising a truncated variant of a natural polypeptide).

In certain cases, the intervening linker polypeptide is a cleavage-susceptible intervening linker polypeptide. In some embodiments, the intervening linker polypeptide is a cleavable or ribosome skipping linker. In some embodiments, the cleavable linker or ribosome skipping linker sequence is selected from the group consisting of 2A, GSG-2A, GSG linker, SGSG linker (SEQ ID NO: 121), furinlink variants and derivatives thereof. In some embodiments, the 2A linker is a p2A linker, a T2A linker, F2A linker or E2A linker. In some embodiments, polypeptides of interest are expressed as fusion proteins linked by a cleavage-susceptible intervening linker polypeptide. In certain embodiments, cleavage-susceptible intervening linker polypeptide(s) can be any one or more of: F/T2A, T2A, p2A, 2A, GSG-p2A, GSG linker, and furinlink variants. Linkers (polynucleotide and polypeptide sequences), such as those disclosed in PCT/US2016/061668 (WO2017083750) published 18-May-2017, which is incorporated by reference herein.

In certain embodiments, the linker polypeptide comprises a sequence disclosed in Table 3.

TABLE 3
Linker amino acid sequences.

| SEQ ID | | |
|---|---|---|
| NO: | Linker Type | Amino Acid Sequence |
| 76 | RIGID LINKER | EAAAK(n) |
| | | (Where n = sequence repeating any |
| | | multiple number of times such as 1, 2, 3, |
| | | 4, 5 times, etc...) |
| 34 | RIGID LINKER | EAAAK(n) (Where n = 2) |
| 40 | HPV6 E6 Epitope/Linker | TAEIYSYAYKQLKVL |
| 42 | HPV11 E6 Epitope/Linker | TAEIYAYAYKNLKVV |
| 45 | HPV11 E7 Epitope/Linker | HCYEQLEDSSEDEVD |
| 52 | HPV16 E7 agonist | RTLEDLLMGV |
| 48 | HPV16 E6 agonist 1 | KLPQLCTEV |
| 50 | HPV16 E6 agonist 2 | QLYNKPLCDV |
| 57 | Flex linker 1 | VSQTSKLTR |

In certain cases, a linker polypeptide can comprise an amino acid sequence “RAKR” (SEQ PG2DN ID NO: 123). In certain cases, a furin intervening linker polypeptide may be encoded by a polynucleotide sequence polynucleotide sequence comprising “CGTGCAAAGCGT” (SEQ ID NO: 125) or “AGAGCTAAGAGG” (SEQ ID NO: 126).

In some embodiments, the intervening linker polypeptide comprises a furin polypeptide and a 2A polypeptide connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), praline (Pro)(P), phenylalanine (Phe)(F), methionine (Met)(M), tryptophan (Trp)(W). In some cases, a polypeptide linker can also include one or more GS linker sequences, for instance (GS)n (SEQ ID NO: 129), (SG)n (SEQ ID NO: 130), (GSG)n (SEQ ID NO: 131), and (SGSG)n (SEQ ID NO: 132), wherein n can be any number from zero to fifteen.

The linkers described herein can, in certain cases, improve biological activity, increase expression yield, and achieving desirable pharmacokinetic profiles. A linker can also comprise hydrazone, peptide, disulfide, or thioester.

Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n (SEQ ID NO: 127). By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers useful in the present invention may be rich in small or polar amino acids such as Gly and Ser to provide good flexibility and solubility. Flexible linkers can be suitable choices when certain movements or interactions are desired for fusion protein domains. In addition, although flexible linkers cannot have rigid structures, they can serve as a passive linker to keep a distance between functional domains. The length of flexible linkers can be adjusted to allow for proper folding or to achieve optimal biological activity of the fusion proteins.

In some cases, the intervening linker polypeptide described herein is a rigid linker. A rigid linker can be utilized to maintain a fixed distance between domains of a polypeptide. Examples of rigid linkers can be: Alpha helix-forming linkers, Pro-rich sequence, (XP)n, X-Pro backbone, A(EAAAK)nA (n=2-5) (SEQ ID NO: 128), to name a few. Rigid linkers can exhibit relatively stiff structures by adopting a-helical structures or by containing multiple Pro residues in some cases.

In some embodiments, the intervening linker polypeptide may be non-cleavable. Non-cleavable linkers can covalently join functional domains together to act as one molecule throughout an in vivo processes or an ex vivo process.

In other embodiments, the intervening linker polypeptide may be cleavable. A cleavable linker can be introduced to release free functional domains in vivo. A cleavable linker can be cleaved by the presence of reducing reagents, proteases, to name a few. For example, a reduction of a disulfide bond can be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein can also be carried out by proteases that can be expressed in vivo under pathological conditions (e.g. cancer or inflammation), in specific cells or tissues, or constrained within certain cellular compartments. In some cases, a cleavable linker can allow for targeted cleavage. For example, the specificity of many proteases can offer slower cleavage of a linker in constrained compartments. A cleavable linker can also comprise hydrazone, peptides, disulfide, or thioester. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation.

Provided are methods of obtaining an improved expression of a polypeptide construct comprising: providing a polynucleotide encoding said polypeptide construct comprising a first functional polypeptide and a second functional polypeptide, wherein said first functional polypeptide and second functional polypeptide are connected by a linker polypeptide comprising a sequence with at least 60% identity to the sequence APVKQ(SEQ ID NO: 133); and expressing said polynucleotide in a host cell, wherein said expressing results in an improved expression of the polypeptide construct as compared to a corresponding polypeptide construct that does not have a linker polypeptide comprising a sequence with at least 60% identity to the sequence APVKQ (SEQ ID NO: 133).

### 4. Engineered and Designed Linkers

In some embodiments, the polynucleotide linker may be engineered or designed. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

### D. Packaging Sequences

In addition to the expression cassette, the vector may further comprise a packaging sequence. As used herein, the term “packaging sequence” refers to sequences located within the gorilla adenoviral genome which are required for insertion of the viral DNA into the viral capsid or particle. See Ostapchuk et al., Curr. Topics in Microbiology and Immunology, 272:165-185 (1995) and Ahi et al., Frontiers in Microbiology, 7:150 (2016).

### E. HPV Early Region Proteins

Constitutive or induced expression of HPV early (E) region proteins provide targets for an effective HPV vaccine.

HPV genes (E1-E8) regulate viral expression and replication, and late (L) genes control viral protein coding (8-10). HPV early region protein functions include the following: E1, E2 have functions in viral replication/transcription (e.g., E2 regulates expression of E6 and E7; and, E1/E2 interaction is essential for viral replication); E4, E5 have increased expression during late stage of viral replication cycle; and, E6, E7 act co-operatively during replication (E6 is required for episomal genome maintenance, E7 expands compartment of epithelial cells active in DNA replication).

An exemplary embodiment of the present invention is an HPV6/11 vaccine that delivers a multi-epitope antigen design containing epitopes of HPV 6 and 11-namely, key immunogenic peptides from E2 (HPV6), E4 (HPV6), E6 (HPV6/11), and E7 (HPV6/11) where the HPV6-derived peptides have high sequence similarity with HPV11.

### F. Adenoviral Expression Cassettes

In some cases where an adenoviral vector is used, the expression cassette may be located at the E1 region deletion junction or the E4 deletion junction. In certain embodiments, the expression cassette is located in the E1 region deletion junction.

In certain embodiments, the expression cassette is cloned in the right-to-left orientation with respect to the adenovirus viral genome.

In certain embodiments, the expression cassette, as cloned in the right-to-left orientation within the adenovirus viral genome, comprises a nucleic acid sequence of SEQ ID NO: 116 or a functional variant thereof (e.g., a nucleic acid having at least 80%, 85%, 90%, 95%, 96%, 97%, 98%, 99%, 99.1%, 99.5%, 99.9%, or 99.99% sequence identity with SEQ ID NO: 116 or a codon degenerate variant of SEQ ID NO: 116, or a conservatively-substituted variant of SEQ ID NO: 116, or a non-conservatively-substituted variant of SEQ ID NO: 116).

### III. Antigenicity Bioinformatics Workflow for HPV Vaccine Designs

### A. Identification of New HPV Antigenic Components

Based on the important role that HPV E2 and E4 genetic components play in HPV essential functions, the location of the corresponding proteins, as well as in-silico prediction, E2- and E4-derived antigens were identified for an HPV therapeutic vaccine. Non-oncogenic and viral inactivation genetic modifications were also applied to eliminate viral and oncogenic biological activity from HPV proteins, such as in HPV E2 and E6 proteins.

Genetic manipulation was also applied to achieve production of protein sequences reordered in such manner as to retain immunogenic features of the peptides, but eliminate oncogenic and viral amplification functions of E7 and E4, respectively.

As such, some of the innovative aspects of the designs exemplified in this specification include: (1) use of gene constructs encoding fusion proteins comprising four or more different HPV proteins; (2) combining amino acid point mutations and overlapping polypeptide sequence shuffling techniques to inactivate oncogenic and essential viral functions; (3) incorporation of HPV proteins comprising multiple antigenic components from HPV proteins which are highly expressed in host infected cells; (4) first known hybrid antigen designs; (5) combining epitopes from high cancer risk and low cancer risk HPV strains; (6) use of mixed and regularly repeating linkers; (7) use of rigid linkers to stabilize polypeptide subunits and prevent undesirable intra-molecular interactions; (8) use of cleavable linkers between epitopes; and (9) dual use of linker sequence to provide both protein-protein linker (-) function as well as antigens and epitopes, per se (i.e., antigenicity conferred by the linker sequences).

Antigenicity is the capacity to stimulate the production of antibodies or cell-mediated immune responses. The antigenicity of the final design sequences was predicted by the Vaxjen software, which is an alignment-independent model for antigen recognition based on main chemical properties of amino acid sequences. The results indicate that the five antigen sequences are antigenic. See, Table 4 (Antigenicity Virus & Tumor). (SEQ ID NO: 121) (SEQ ID NO: 123) In certain embodiments, two or more polypeptides encoded by a polynucleotide described herein can be separated by an intervening sequence encoding a linker polypeptide. In certain cases, the linker is a cleavage-susceptible linker. In some embodiments, polypeptides of interest are expressed as fusion proteins linked by a cleavage-susceptible linker polypeptide. In certain embodiments, cleavage-susceptible linker polypeptide(s) can be any one or two of: Furinlink, fmdv, p2a, GSG-p2a, and/or fp2a described below. In some cases, a linker is APVKQGSG (SEQ ID NO: 124).

Allergens are small antigens that commonly provoke an antibody response. Allergenicity, whether the antigen is an allergen or non-allergen was predicted by ALLERTOP, a bioinformatics-based allergen prediction software with machine learning methods for classification. It includes logistic regression (LR), decision tree (DT), naive Bayes (NB), random forest (RF), multilayer perceptron (MLP), and k nearest neighbors (kNN). The results indicate that the five antigen sequences are non-allergenic. See, Table 4 (Allergenicity).

Cross-reactivity or invocation of autoimmune side effects in various tissues has important safety implications in adoptive immunotherapy. Sequence homology analyses were performed to assess if those novel antigens have cross-reactivity with human proteome with blast search, basic local alignment search tool. No host cross reactivity was identified in these five antigen sequences. See, Table 4 (Host Cross-Reactivity).

### B. Software/Tools

Software tools utilized in performance of the designs described herein include, but are not limited to:
ALLERTOP (See, AllerTOP v.2—a server for in silico prediction of allergens; J Mol Model. 2014 June; 20(6):2278. doi: 10.1007/s00894-014-2278-5. Epub 2014 May 31.)
ANN (See, Reliable prediction of T-cell epitopes using neural networks with novel sequence representations; Protein Sci. 2003 May; 12(5):1007-17.)
BLAST (Basic Local Alignment Search Tool; NCBI, National Center for Biotechnology Information, U.S. National Library of Medicine 8600 Rockville Pike, Bethesda MD, 20894 USA.)
CLUSTALW2 (EMBL-EBI, Wellcome Genome Campus, Hinxton, Cambridgeshire, CB10 1SD, UK. +44 (0)1223 49 44 44.)
GENEIOUS VI 1.1.5 (See, geneious.com/biopharma/)
IEDB CONSENSUS (See, A consensus epitope prediction approach identifies the breadth of murine T(CD8+)-cell responses to vaccinia virus; Nat Biotechnol. 2006 July; 24(7):817-9. Epub 2006 Jun. 11)
NETMHCPAN 4.0 (See, Gapped sequence alignment using artificial neural networks: application to the MHC class I system; Bioinformatics. 2016 Feb. 15; 32(4):511-7. doi: 10.1093/bioinformatics/btv639. Epub 2015 Oct. 29.)
PHYRE2 (See, The Phyre2 web portal for protein modeling, prediction and analysis; 7 May 2015; nature.com/articles/nprot.2015.053; doi.org/10.1038/nprot.2015.053.)
PYMOL MOLECULAR GRAPHICS SYSTEM V2. 1. 1 (See, pymol.org/2/;sourceforge.net/projects/pymol/support)
VAXJEN (See, VaxiJen: a server for prediction of protective antigens, tumour antigens and subunit vaccines; BMC Bioinformatics. 2007 Jan. 5; 8:4 . . . )

### III. HPV Antigen Design and Variants Thereof

In an exemplary embodiments, the polynucleotide encoding a fusion protein (e.g., an HPV antigen) comprises two or more HPV proteins. For example, the polynucleotide encoding a fusion protein comprises one or more HPV6 proteins, one or more HPV11 proteins, and one or more HPV45 proteins (e.g., an HPV6 protein and an HPV11 protein).

Exemplary HPV6 proteins include, but are not limited to, one or more HPV6 E2 proteins, one or more HPV6 E4 proteins, one or more HPV6 E6 proteins, one or more HPV6 E7 proteins, and combinations thereof, including but not limited to combinations of HPV6 and/or HPV11 protein types and multiple copies or variants of a single HPV6 and/or HPV11 protein.

In some embodiments, the HPV6 E2 protein comprises an amino acid sequence having at least 80% sequence identity (e.g., 85%, 90%, 95%, 97%, 98%, 99%, or 99.5% sequence identity) with SEQ ID NO: 1. In some embodiments, the HPV6 E2 protein comprises an amino acid sequence having at least 90% sequence identity with SEQ ID NO: 1. In some embodiments, the HPV6 E2 protein comprises an amino acid sequence having at least 95% sequence identity with SEQ ID NO: 1. In some embodiments, the HPV6 E2 protein comprises an amino acid sequence having 1-36 amino acid substitutions (e.g., 1-30, 1-25, 1-20, 1-15, 1-10, or 1-5 amino acid substitutions) as compared to SEQ ID NO: 1. In some embodiments, the HPV6 E2 protein comprises the amino acid sequence of SEQ ID NO: 1. In some embodiments, an HPV E2 protein comprises an amino acid sequence having at least 80% sequence identity (e.g., 85%, 90%, 95%, 97%, 98%, 99%, or 99.5% sequence identity) with SEQ ID NO: 105. In some embodiments, the HPV E2 protein comprises an amino acid sequence having at least 90% sequence identity with SEQ ID NO: 105.

In some embodiments, the HPV6 E4 protein comprises an amino acid sequence having at least 80% sequence identity (e.g., 85%, 90%, 95%, 97%, 98%, 99%, or 99.5% sequence identity) with SEQ ID NO: 3 or 7. In some embodiments, the HPV6 E4 protein comprises an amino acid sequence having at least 90% sequence identity with SEQ ID NO: 3 or 7. In some embodiments, the HPV6 E4 protein comprises an amino acid sequence having at least 95% sequence identity with SEQ ID NO: 3 or 7. In some embodiments, the HPV6 E4 protein comprises an amino acid sequence having 1-5 amino acid substitutions (e.g., 1-4, 1-3, 1-2, or 1 amino acid substitutions) as compared to SEQ ID NO: 3 or 7. In some embodiments, the HPV6 E4 protein comprises an amino acid sequence having at least 80% sequence identity (e.g., 85%, 90%, 95%, 97%, 98%, 99%, or 99.5% sequence identity) with SEQ ID NO: 3. In some embodiments, the HPV6 E4 protein comprises an amino acid sequence having at least 90% sequence identity with SEQ ID NO: 3. In some embodiments, the HPV6 E4 protein comprises an amino acid sequence having at least 95% sequence identity with SEQ ID NO: 3. In some embodiments, the HPV6 E4 protein comprises an amino acid sequence having 1-5 amino acid substitutions (e.g., 1-4, 1-3, 1-2, or 1 amino acid substitutions) as compared to SEQ ID NO: 3. In some embodiments, the HPV6 E4 protein comprises an amino acid sequence having at least 80% sequence identity (e.g., 85%, 90%, 95%, 97%, 98%, 99%, or 99.5% sequence identity) with SEQ ID NO: 7. In some embodiments, the HPV6 E4 protein comprises an amino acid sequence having at least 90% sequence identity with SEQ ID NO: 7. In some embodiments, the HPV6 E4 protein comprises an amino acid sequence having at least 95% sequence identity with SEQ ID NO: 7. In some embodiments, the HPV6 E4 protein comprises an amino acid sequence having 1-5 amino acid substitutions (e.g., 1-4, 1-3, 1-2, or 1 amino acid substitutions) as compared to SEQ ID NO: 7. In some embodiments, the HPV6 E4 protein comprises the amino acid sequence of SEQ ID NO: 3 or 7. In some embodiments, the HPV6 E4 protein comprises the amino acid sequence of SEQ ID NO: 3. In some embodiments, the HPV6 E4 protein comprises the amino acid sequence of SEQ ID NO: 7. In some embodiments, the HPV E4 protein comprises an amino acid sequence selected from SEQ ID Nos: 3, 7, 107, 111, and 172-179. In some embodiments, the HPV E4 protein comprises an amino acid sequence selected from SEQ ID Nos: 3 and 7.

In some embodiments, the HPV6 E6 protein comprises an amino acid sequence having at least 80% sequence identity (e.g., 85%, 90%, 95%, 97%, 98%, 99%, or 99.5% sequence identity) with SEQ ID NO: 11 or 40. In some embodiments, the HPV6 E6 protein comprises an amino acid sequence having at least 90% sequence identity with SEQ ID NO: 11 or 40. In some embodiments, the HPV6 E6 protein comprises an amino acid sequence having at least 95% sequence identity with SEQ ID NO: 11 or 40. In some embodiments, the HPV6 E6 protein comprises an amino acid sequence having 1-5 amino acid substitutions (e.g., 1-4, 1-3, 1-2, or 1 amino acid substitution(s)) as compared to SEQ ID NO: 11 or 40. In some embodiments, the HPV6 E6 protein comprises an amino acid sequence having at least 80% sequence identity (e.g., 85%, 90%, 95%, 97%, 98%, 99%, or 99.5% sequence identity) with SEQ ID NO: 11. In some embodiments, the HPV6 E6 protein comprises an amino acid sequence having at least 90% sequence identity with SEQ ID NO: 11. In some embodiments, the HPV6 E6 protein comprises an amino acid sequence having at least 95% sequence identity with SEQ ID NO: 11. In some embodiments, the HPV6 E6 protein comprises an amino acid sequence having 1-5 amino acid substitutions (e.g., 1-4, 1-3, 1-2, or 1 amino acid substitution(s)) as compared to SEQ ID NO: 11. In some embodiments, the HPV6 E6 protein comprises an amino acid sequence having at least 90% sequence identity with SEQ ID NO: 40. In some embodiments, the HPV6 E6 protein comprises an amino acid sequence having at least 95% sequence identity with SEQ ID NO: 40. In some embodiments, the HPV6 E6 protein comprises an amino acid sequence having 1-5 amino acid substitutions (e.g., 1-4, 1-3, 1-2, or 1 amino acid substitution(s)) as compared to SEQ ID NO: 40. In some embodiments, the HPV6 E6 protein comprises the amino acid sequence of SEQ ID NO: 11 or 40. In some embodiments, the HPV6 E6 protein comprises the amino acid sequence of SEQ ID NO: 11. In some embodiments, the HPV6 E6 protein comprises the amino acid sequence of SEQ ID NO: 40. In some embodiments, the HPV6 E6 protein comprises an amino acid sequence having at least 80% sequence identity (e.g., 85%, 90%, 95%, 97%, 98%, 99%, or 99.5% sequence identity) with SEQ ID NO: 110. In some embodiments, the HPV6 E6 protein comprises an amino acid sequence having at least 90% sequence identity with SEQ ID NO: 110. In some embodiments, the HPV6 E6 protein comprises an amino acid sequence having at least 95% sequence identity with SEQ ID NO: 110. In some embodiments, the HPV6 E6 protein comprises an amino acid sequence having 1-5 amino acid substitutions (e.g., 1-4, 1-3, 1-2, or 1 amino acid substitution(s)) as compared to SEQ ID NO: 110. In some embodiments, the HPV6 E6 protein comprises the amino acid sequence of SEQ ID NO: 110. In some embodiments, the HPV6 E6 protein comprises an amino acid sequence selected from SEQ ID NOs: 11, 40, 110, and 197-204. In some embodiments, the HPV6 E6 protein comprises an amino acid sequence selected from SEQ ID NOs: 11, 40, and 110.

In some embodiments, the HPV6 E7 protein comprises an amino acid sequence having at least 80% sequence identity (e.g., 85%, 90%, 95%, 97%, 98%, 99%, or 99.5% sequence identity) with SEQ ID NO: 5 or 9. In some embodiments, the HPV6 E7 protein comprises an amino acid sequence having at least 90% sequence identity with SEQ ID NO: 5 or 9. In some embodiments, the HPV6 E7 protein comprises an amino acid sequence having at least 95% sequence identity with SEQ ID NO: 5 or 9. In some embodiments, the HPV6 E7 protein comprises an amino acid sequence having 1-10 amino acid substitutions (e.g., 1-9, 1-8, 1-7, 1-6, 1-5, 1-4, 1-3, 1-2, or 1 amino acid substitution(s)) as compared to SEQ ID NO: 5 or 9. In some embodiments, the HPV6 E7 protein comprises an amino acid sequence having at least 80% sequence identity (e.g., 85%, 90%, 95%, 97%, 98%, 99%, or 99.5% sequence identity) with SEQ ID NO: 5. In some embodiments, the HPV6 E7 protein comprises an amino acid sequence having at least 90% sequence identity with SEQ ID NO: 5. In some embodiments, the HPV6 E7 protein comprises an amino acid sequence having at least 95% sequence identity with SEQ ID NO: 5. In some embodiments, the HPV6 E7 protein comprises an amino acid sequence having 1-10 amino acid substitutions (e.g., 1-9, 1-8, 1-7, 1-6, 1-5, 1-4, 1-3, 1-2, or 1 amino acid substitution(s)) as compared to SEQ ID NO: 5. In some embodiments, the HPV6 E7 protein comprises the amino acid sequence of SEQ ID NO: 5. In some embodiments, the HPV6 E7 protein comprises an amino acid sequence having at least 80% sequence identity (e.g., 85%, 90%, 95%, 97%, 98%, 99%, or 99.5% sequence identity) with SEQ ID NO: 9. In some embodiments, the HPV6 E7 protein comprises an amino acid sequence having at least 90% sequence identity with SEQ ID NO: 9. In some embodiments, the HPV6 E7 protein comprises an amino acid sequence having at least 95% sequence identity with SEQ ID NO: 9. In some embodiments, the HPV6 E7 protein comprises an amino acid sequence having 1-10 amino acid substitutions (e.g., 1-9, 1-8, 1-7, 1-6, 1-5, 1-4, 1-3, 1-2, or 1 amino acid substitution(s)) as compared to SEQ ID NO: 9. In some embodiments, the HPV6 E7 protein comprises the amino acid sequence of SEQ ID NO: 9. In some embodiments, the HPV6 E7 protein comprises the amino acid sequence of SEQ ID NO: 5 or 9. In some embodiments, the HPV6 E7 protein comprises an amino acid sequence having at least 80% sequence identity (e.g., 85%, 90%, 95%, 97%, 98%, 99%, or 99.5% sequence identity) with SEQ ID NO: 109. In some embodiments, the HPV6 E7 protein comprises an amino acid sequence having at least 90% sequence identity with SEQ ID NO: 109. In some embodiments, the HPV6 E7 protein comprises an amino acid sequence having at least 95% sequence identity with SEQ ID NO: 109. In some embodiments, the HPV6 E7 protein comprises an amino acid sequence having 1-10 amino acid substitutions (e.g., 1-9, 1-8, 1-7, 1-6, 1-5, 1-4, 1-3, 1-2, or 1 amino acid substitution(s)) as compared to SEQ ID NO: 109. In some embodiments, the HPV6 E7 protein comprises the amino acid sequence of SEQ ID NO: 109. In some embodiments, the HPV6 E7 protein comprises an amino acid sequence selected from SEQ ID Nos: 5, 9, 109, and 189-196. In some embodiments, the HPV6 E7 protein comprises an amino acid sequence selected from SEQ ID Nos: 5, 9, and 109.

Exemplary HPV11 proteins include, but are not limited to, one or more HPV11 E6 proteins and one or more HPV11 E7 proteins. In some embodiments, the HPV11 E6 protein comprises an amino acid sequence having at least 80% sequence identity (e.g., 85%, 90%, 95%, 97%, 98%, 99%, or 99.5% sequence identity) with SEQ ID NO: 42. In some embodiments, the HPV11 E6 protein comprises an amino acid sequence having at least 90% sequence identity with SEQ ID NO: 42. In some embodiments, the HPV11 E6 protein comprises an amino acid sequence having at least 95% sequence identity with SEQ ID NO: 42. In some embodiments, the HPV11 E6 protein comprises an amino acid sequence having 1-5 amino acid substitutions (e.g., 1-4, 1-3, 1-2, or 1 amino acid substitution(s)) as compared to SEQ ID NO: 42. In some embodiments, the HPV11 E6 protein comprises the amino acid sequence of SEQ ID NO: 42. In some embodiments, the HPV11 E6 protein comprises an amino acid sequence having at least 80% sequence identity (e.g., 85%, 90%, 95%, 97%, 98%, 99%, or 99.5% sequence identity) with SEQ ID NO: 108. In some embodiments, the HPV11 E6 protein comprises an amino acid sequence having at least 90% sequence identity with SEQ ID NO: 108. In some embodiments, the HPV11 E6 protein comprises an amino acid sequence having at least 95% sequence identity with SEQ ID NO: 108. In some embodiments, the HPV11 E6 protein comprises an amino acid sequence having 1-5 amino acid substitutions (e.g., 1-4, 1-3, 1-2, or 1 amino acid substitution(s)) as compared to SEQ ID NO: 108. In some embodiments, the HPV11 E6 protein comprises the amino acid sequence of SEQ ID NO: 108. In some embodiments, the HPV11 E6 protein comprises an amino acid sequence having at least 80% sequence identity (e.g., 85%, 90%, 95%, 97%, 98%, 99%, or 99.5% sequence identity) with SEQ ID NO: 112. In some embodiments, the HPV11 E6 protein comprises an amino acid sequence having at least 90% sequence identity with SEQ ID NO: 112. In some embodiments, the HPV11 E6 protein comprises an amino acid sequence having at least 95% sequence identity with SEQ ID NO: 112. In some embodiments, the HPV11 E6 protein comprises an amino acid sequence having 1-5 amino acid substitutions (e.g., 1-4, 1-3, 1-2, or 1 amino acid substitution(s)) as compared to SEQ ID NO: 112. In some embodiments, the HPV11 E6 protein comprises the amino acid sequence of SEQ ID NO: 112. In some embodiments, the HPV11 E6 protein comprises an amino acid sequence selected from SEQ ID Nos: 42, 108, 112, 180-188, and 213-220. In some embodiments, the HPV11 E6 protein comprises an amino acid sequence selected from SEQ ID NOs: 42, 108, and 112.

In some embodiments, the HPV11 E7 protein comprises an amino acid sequence having at least 80% sequence identity (e.g., 85%, 90%, 95%, 97%, 98%, 99%, or 99.5% sequence identity) with SEQ ID NO: 45 or 106. In some embodiments, the HPV11 E7 protein comprises an amino acid sequence having at least 90% sequence identity with SEQ ID NO: 45 or 106. In some embodiments, the HPV11 E7 protein comprises an amino acid sequence having at least 95% sequence identity with SEQ ID NO: 45 or 106. In some embodiments, the HPV11 E7 protein comprises an amino acid sequence having 1-5 amino acid substitutions (e.g., 1-4, 1-3, 1-2, or 1 amino acid substitution(s)) as compared to SEQ ID NO: 45 or 106. In some embodiments, the HPV11 E7 protein comprises an amino acid sequence having at least 80% sequence identity (e.g., 85%, 90%, 95%, 97%, 98%, 99%, or 99.5% sequence identity) with SEQ ID NO: 45. In some embodiments, the HPV11 E7 protein comprises an amino acid sequence having at least 90% sequence identity with SEQ ID NO: 45. In some embodiments, the HPV11 E7 protein comprises an amino acid sequence having at least 95% sequence identity with SEQ ID NO: 45. In some embodiments, the HPV11 E7 protein comprises an amino acid sequence having 1-5 amino acid substitutions (e.g., 1-4, 1-3, 1-2, or 1 amino acid substitution(s)) as compared to SEQ ID NO: 45. In some embodiments, the HPV11 E7 protein comprises the amino acid sequence of SEQ ID NO: 45. In some embodiments, the HPV11 E7 protein comprises an amino acid sequence having at least 80% sequence identity (e.g., 85%, 90%, 95%, 97%, 98%, 99%, or 99.5% sequence identity) with SEQ ID NO: 106. In some embodiments, the HPV11 E7 protein comprises an amino acid sequence having at least 90% sequence identity with SEQ ID NO: 106. In some embodiments, the HPV11 E7 protein comprises an amino acid sequence having at least 95% sequence identity with SEQ ID NO: 106. In some embodiments, the HPV11 E7 protein comprises an amino acid sequence having 1-5 amino acid substitutions (e.g., 1-4, 1-3, 1-2, or 1 amino acid substitution(s)) as compared to SEQ ID NO: 106. In some embodiments, the HPV11 E7 protein comprises the amino acid sequence of SEQ ID NO: 106. In some embodiments, the HPV11 E7 protein comprises an amino acid sequence having at least 80% sequence identity (e.g., 85%, 90%, 95%, 97%, 98%, 99%, or 99.5% sequence identity) with SEQ ID NO: 114. In some embodiments, the HPV11 E7 protein comprises an amino acid sequence having at least 90% sequence identity with SEQ ID NO: 114. In some embodiments, the HPV11 E7 protein comprises an amino acid sequence having at least 95% sequence identity with SEQ ID NO: 114. In some embodiments, the HPV11 E7 protein comprises an amino acid sequence having 1-5 amino acid substitutions (e.g., 1-4, 1-3, 1-2, or 1 amino acid substitution(s)) as compared to SEQ ID NO: 114. In some embodiments, the HPV11 E7 protein comprises the amino acid sequence of SEQ ID NO: 114. In some embodiments, the HPV11 E7 protein comprises an amino acid sequence selected from SEQ ID Nos: 45, 106, 114, 164-171, and 229-236. In some embodiments, the HPV11 E7 protein comprises an amino acid sequence selected from SEQ ID Nos: 45, 106, and 114.

A consensus sequence between the HPV6 E2 and the HPV11 E2 protein sequences or fragments thereof can be determined, and can be referred to an HPV E2 protein. In some embodiments, the HPV E2 protein comprises an amino acid sequence having at least 80% sequence identity (e.g., 85%, 90%, 95%, 97%, 98%, 99%, or 99.5% sequence identity) with SEQ ID NO: 105. In some embodiments, the HPV E2 protein comprises an amino acid sequence having at least 90% sequence identity with SEQ ID NO: 105. In some embodiments, the HPV E2 protein comprises an amino acid sequence having at least 95% sequence identity with SEQ ID NO: 105. In some embodiments, the HPV E2 protein comprises an amino acid sequence having 1-36 amino acid substitutions (e.g., 1-30, 1-25, 1-20, 1-15, 1-10, or 1-5 amino acid substitutions) as compared to SEQ ID NO: 105. In some embodiments, the HPV E2 protein comprises an amino acid sequence selected from SEQ ID Nos: 105, and 154-163.

A consensus sequence between the HPV6 E4 and the HPV11 E4 protein sequences or fragments thereof can be determined, and can be referred to an HPV E4 protein, which can be included in any of the polynucleotide or fusion proteins described herein. In some embodiments, the HPV E4 protein comprises an amino acid sequence having at least 80% sequence identity (e.g., 85%, 90%, 95%, 97%, 98%, 99%, or 99.5% sequence identity) with SEQ ID NO: 107. In some embodiments, the HPV E4 protein comprises an amino acid sequence having at least 90% sequence identity with SEQ ID NO: 107. In some embodiments, the HPV E4 protein comprises an amino acid sequence having at least 95% sequence identity with SEQ ID NO: 107. In some embodiments, the HPV E4 protein comprises an amino acid sequence having 1-5 amino acid substitutions (e.g., 1-4, 1-3, 1-2, or 1 amino acid substitutions) as compared to SEQ ID NO: 107. In some embodiments, the HPV6 E4 protein comprises the amino acid sequence of SEQ ID NO: 107. In some embodiments, the HPV E4 protein comprises an amino acid sequence having at least 80% sequence identity (e.g., 85%, 90%, 95%, 97%, 98%, 99%, or 99.5% sequence identity) with SEQ ID NO: 111. In some embodiments, the HPV E4 protein comprises an amino acid sequence having at least 90% sequence identity with SEQ ID NO: 111. In some embodiments, the HPV E4 protein comprises an amino acid sequence having at least 95% sequence identity with SEQ ID NO: 111. In some embodiments, the HPV E4 protein comprises an amino acid sequence having 1-5 amino acid substitutions (e.g., 1-4, 1-3, 1-2, or 1 amino acid substitutions) as compared to SEQ ID NO: 111. In some embodiments, the HPV6 E4 protein comprises the amino acid sequence of SEQ ID NO: 111. In some embodiments, the HPV6 E4 protein comprises an amino acid sequence selected from SEQ ID NOs: 107, 111, 172-179, and 205-212. In some embodiments, the HPV6 E4 protein comprises an amino acid sequence selected from SEQ ID NOs: 107 and 111.

A consensus sequence between the HPV6 E6 and the HPV11 E6 protein sequences or fragments thereof can be determined, and can be referred to an HPV E6 protein, which can be included in any of the polynucleotide or fusion proteins disclosed herein. In some embodiments, the HPV E6 protein comprises an amino acid sequence having at least 80% sequence identity (e.g., 85%, 90%, 95%, 97%, 98%, 99%, or 99.5% sequence identity) with SEQ ID NO: 115. In some embodiments, the HPV E6 protein comprises an amino acid sequence having at least 90% sequence identity with SEQ ID NO: 115. In some embodiments, the HPV E6 protein comprises an amino acid sequence having at least 95% sequence identity with SEQ ID NO: 115. In some embodiments, the HPV E6 protein comprises an amino acid sequence having 1-5 amino acid substitutions (e.g., 1-4, 1-3, 1-2, or 1 amino acid substitution(s)) as compared to SEQ ID NO: 115. In some embodiments, the HPV E6 protein comprises the amino acid sequence of SEQ ID NO: 115. In some embodiments, the HPV E6 protein comprises an amino acid sequence selected from SEQ ID NO: 115 and 237-246.

A consensus sequence between the HPV6 E7 and the HPV11 E7 protein sequences or fragments thereof can be determined, and can be referred to an HPV E7 protein, which can be included in any of the polynucleotide or fusion proteins disclosed herein. In some embodiments, the HPV E7 protein comprises an amino acid sequence having at least 80% sequence identity (e.g., 85%, 90%, 95%, 97%, 98%, 99%, or 99.5% sequence identity) with SEQ ID NO: 113. Exemplary variants include amino acid sequences of SEQ ID NO: 221-228. In some embodiments, the HPV E7 protein comprises an amino acid sequence having at least 90% sequence identity with SEQ ID NO: 113. In some embodiments, the HPV E7 protein comprises an amino acid sequence having at least 95% sequence identity with SEQ ID NO: 113. In some embodiments, the HPV E7 protein comprises an amino acid sequence having 1-5 amino acid substitutions (e.g., 1-4, 1-3, 1-2, or 1 amino acid substitution(s)) as compared to SEQ ID NO: 113. In some embodiments, the HPV E7 protein comprises the amino acid sequence of SEQ ID NO: 113. In some embodiments, the HPV E7 protein comprises an amino acid sequence selected from SEQ ID NO: 113, and 221-228.

In some embodiments, a fusion protein includes one or more copies of an HPV6 and/or and HPV11 protein. For example, a fusion protein includes one or more copies of an HPV6 E4 protein, an HPV6 E6 protein, an HPV6 E7 protein, an HPV11 E6 protein or an HPV11 E7 protein. In some embodiments, the fusion protein includes an HPV6 E4 protein comprising an amino acid sequence having at least 80% sequence identity (e.g., 85%, 90%, 95%, 97%, 98%, 99%, or 99.5% sequence identity) with SEQ ID NO: 3 and an HPV6 E4 protein comprising an amino acid sequence having at least 80% sequence identity (e.g., 85%, 90%, 95%, 97%, 98%, 99%, or 99.5% sequence identity) with SEQ ID NO: 7. In some embodiments, the fusion protein includes an HPV6 E4 protein comprising an amino acid sequence having at least 90% sequence identity with SEQ ID NO: 3 and an HPV6 E4 protein comprising an amino acid sequence having at least 90% sequence identity with SEQ ID NO: 7. In some embodiments, the fusion protein includes an HPV6 E4 protein comprising an amino acid sequence having at least 95% sequence identity with SEQ ID NO: 3 and an HPV6 E4 protein comprising an amino acid sequence having at least 95% sequence identity with SEQ ID NO: 7. In some embodiments, the fusion protein includes an HPV6 E4 protein comprising the amino acid sequence of SEQ ID NO: 3 and an HPV6 E4 protein comprising the amino acid sequence of SEQ ID NO: 7. In some embodiments, the fusion protein comprises an HPV6 E6 protein comprising an amino acid sequence having at least 80% sequence identity (e.g., 85%, 90%, 95%, 97%, 98%, 99%, or 99.5% sequence identity) with SEQ ID NO: 11 and an HPV6 E6 protein comprising an amino acid sequence having at least 80% sequence identity (e.g., 85%, 90%, 95%, 97%, 98%, 99%, or 99.5% sequence identity) with SEQ ID NO: 40. In some embodiments, the fusion protein comprises an HPV6 E6 protein comprising an amino acid sequence having at least 90% sequence identity with SEQ ID NO: 11 and an HPV6 E6 protein comprising an amino acid sequence having at least 90% sequence identity with SEQ ID NO: 40. In some embodiments, the fusion protein includes an HPV6 E6 protein comprising an amino acid sequence having at least 95% sequence identity with SEQ ID NO: 11 and an HPV6 E6 protein comprising an amino acid sequence having at least 95% sequence identity with SEQ ID NO: 40. In some embodiments, the fusion protein comprises an HPV6 E6 protein comprising the amino acid sequence of SEQ ID NO: 11 and an HPV6 E6 protein comprising the amino acid sequence of SEQ ID NO: 40. In some embodiments, the fusion protein comprises an HPV6 E7 protein comprising an amino acid sequence having at least 80% sequence identity (e.g., 85%, 90%, 95%, 97%, 98%, 99%, or 99.5% sequence identity) with SEQ ID NO: 5 and an HPV6 E7 protein comprising an amino acid sequence having at least 80% sequence identity (e.g., 85%, 90%, 95%, 97%, 98%, 99%, or 99.5% sequence identity) with SEQ ID NO: 9. In some embodiments, the fusion protein comprises an HPV6 E7 protein comprising an amino acid sequence having at least 90% sequence identity with SEQ ID NO: 5 and an HPV6 E7 protein comprising an amino acid sequence having at least 90% sequence identity with SEQ ID NO: 9. In some embodiments, the fusion protein comprises an HPV6 E7 protein comprising an amino acid sequence having at least 95% sequence identity with SEQ ID NO: 5 and an HPV6 E7 protein comprising an amino acid sequence having at least 95% sequence identity with SEQ ID NO: 9. In some embodiments, the fusion protein comprises an HPV6 E7 protein comprising the amino acid sequence of SEQ ID NO: 5 and an HPV6 E7 protein comprising the amino acid sequence of SEQ ID NO: 9.

In an exemplary embodiment, the polypeptide construct or fusion protein encoded by the polynucleotide of the present invention has comprises an amino acid sequence of SEQ ID NO: 66, 68, 70, 72, or 74 or a functional variant thereof (e.g., a nucleic acid having at least 80%, 85%, 90%, 95%, 96%, 97%, 98%, 99%, 99.1%, 99.5%, 99.9%, or 99.99% sequence identity with SEQ ID NO: 66, 68, 70, 72, or 74, or a conservatively-substituted variant of SEQ ID NO: 66, 68, 70, 72, or 74). In some embodiments, the fusion protein comprises an amino acid sequence having at least 80% identity (e.g., at least about 80%, 85%, 90%, 95%, 96%, 97%, 98%, 99%, 99.5%, 99.9%, or 100% identity) with SEQ ID NO: 68. In some embodiments, the fusion protein comprises an amino acid sequence having at least 80% identity (e.g., at least about 80%, 85%, 90%, 95%, 96%, 97%, 98%, 99%, 99.5%, 99.9%, or 100% identity) with SEQ ID NO: 66. In some embodiments, the fusion protein comprises an amino acid sequence having at least 80% identity (e.g., at least about 80%, 85%, 90%, 95%, 96%, 97%, 98%, 99%, 99.5%, 99.9%, or 100% identity) with SEQ ID NO: 70. In some embodiments, the fusion protein comprises an amino acid sequence having at least 80% identity (e.g., at least about 80%, 85%, 90%, 95%, 96%, 97%, 98%, 99%, 99.5%, 99.9%, or 100% identity) with SEQ ID NO: 72. In some embodiments, the fusion protein comprises an amino acid sequence having at least 80% identity (e.g., at least about 80%, 85%, 90%, 95%, 96%, 97%, 98%, 99%, 99.5%, 99.9%, or 100% identity) with SEQ ID NO: 74.

In some embodiments, the fusion protein comprises an amino acid sequence having at least 80% identity with SEQ ID NO: 68. In some embodiments, the fusion protein comprises an amino acid sequence having at least 85% identity with SEQ ID NO: 68. In some embodiments, the fusion protein comprises an amino acid sequence having at least 90% identity with SEQ ID NO: 68. In some embodiments, the fusion protein comprises an amino acid sequence having at least 95% identity with SEQ ID NO: 68. In some embodiments, the fusion protein comprises an amino acid sequence having at least 96% identity with SEQ ID NO: 68. In some embodiments, the fusion protein comprises an amino acid sequence having at least 97% identity with SEQ ID NO: 68. In some embodiments, the fusion protein comprises an amino acid sequence having at least 98% identity with SEQ ID NO: 68. In some embodiments, the fusion protein comprises an amino acid sequence having at least 99% identity with SEQ ID NO: 68. In some embodiments, the fusion protein comprises an amino acid sequence of SEQ ID NO: 68.

In an exemplary embodiment, the polypeptide construct of the present invention comprises a sequence of SEQ ID NO: 68 or a functional variant thereof (e.g., an amino acid sequence having at least 80%, 85%, 90%, 95%, 96%, 97%, 98%, 99%, 99.1%, 99.5%, 99.9%, or 99.99% sequence identity with SEQ ID NO: 68, or a conservatively-substituted variant of SEQ ID NO: 68, or a non-conservatively-substituted variant of SEQ ID NO: 68).

In certain embodiments, the polypeptide construct of the present invention has a functional variant of SEQ ID NO: 68 that, when compared to SEQ ID NO: 68, has similar or enhanced binding affinity to HPV6/11-associated proteins and/or effects a similar or enhanced immunogenic response. Such a variant can be readily determined by sequence alignment software such as ClustalW.

In certain embodiments, the polypeptide construct of the present invention comprises any one of SEQ ID NO: 105-115 or a functional variant thereof (e.g., a nucleic acid having at least 80%, 85%, 90%, 95%, 96%, 97%, 98%, 99%, 99.1%, 99.5%, 99.9%, or 99.99% sequence identity with any one of SEQ ID NO: 105-115, or a conservatively-substituted variant of any one of SEQ ID NO: 105-115, or a non-conservatively-substituted variant of any one of SEQ ID NO: 105-115). In an exemplary embodiment, the polypeptide construct of the present invention comprises each of SEQ ID NO: 105-115.

In some embodiments, a polynucleotide construct variant can contain one or more conservatively-substituted variants of any of the antigen regions having SEQ ID NO: 105-115. For example, any hydrophilic amino acid can be substituted with any other hydrophilic amino acid, any aliphatic amino acid can be substituted with any other aliphatic amino acid, any basic amino acid can be substituted with any other basic amino acid, any aromatic amino acid can be substituted with any other aromatic amino acid, and any combination thereof. Exemplary polynucleotide construct variants with conservatively-substituted mutations include, but are not limited to, SEQ ID NO: 143 and SEQ ID NO: 144.

In some embodiments, a polynucleotide construct variant can contain one or more amino acid additions or deletions. For example, one or more amino acids can be added to or removed from any of the antigen regions of a polynucleotide construct described herein. Exemplary polynucleotide construct variants with amino acid additions and/or deletions include, but are not limited to, SEQ ID NO: 144-148.

In certain embodiments, the polypeptide construct variant has the same HPV6/11 antigen regions as SEQ ID NO: 68, but the antigen regions of SEQ ID NO: 68 are shuffled in an order different from the order of antigen regions of SEQ ID NO: 68. In certain embodiments, the variant comprises the HPV E2 (SEQ ID NO: 105), HPV11 E7 (SEQ ID NO: 106), HPV E4 (SEQ ID NO: 107), HPV11 E6 (SEQ ID NO: 108), HPV6 E7 (SEQ ID NO: 109), HPV6 E6 (SEQ ID NO: 110), HPV E4 (SEQ ID NO: 111), HPV11 E6 (SEQ ID NO: 112), HPV E7 (SEQ ID NO: 113), HPV11 E7 (SEQ ID NO: 114), and HPV E6 (SEQ ID NO: 115) antigen regions of SEQ ID NO: 68, but the antigen regions are shuffled compared to the order of antigen regions of SEQ ID NO: 68. Exemplary polypeptide construct variant sequence include, but are not limited to, SEQ ID NO: 134 (Antigen region order: HPV E4, HPV11 E6, HPV6 E7, HPV6 E6, HPV E4, HPV11 E6, HPV E7, HPV11 E7, HPV E6, HPV E2, and HPV11 E7), SEQ ID NO: 135 (Antigen region order: HPV E6, HPV11 E7, HPV E2, HPV11 E7, HPV E4, HPV11 E6, HPV6 E7, HPV6 E6, HPV E4, HPV11 E6, and HPV E7), and SEQ ID NO: 136 (Antigen region order: HPV E7, HPV11 E7, HPV E6, HPV E4, HPV11 E6, HPV6 E7, HPV6 E6, HPV E4, HPV11 E6, HPV E2, and HPV11 E7).

A polypeptide construct contains from about 2 to about 20 antigen regions (e.g., from about 5 to about 15, from about 10 to about 12). For example, a polypeptide construct contains 2, 3, 4, 5, 6, 7, 8, 9, 10, 11, 12, 13, 14, 15, 16, 17, 18, 19, 20 or more antigen regions. In certain embodiments, a polypeptide construct variant has fewer antigen regions as compared to SEQ ID NO: 68. For example, a polypeptide construct variant has 1, 2, 3, 4, 5, 6, 7, 8, 9, or 10 fewer antigen regions compared to SEQ ID NO: 68. A polypeptide construct variant containing fewer antigen regions compared to SEQ ID NO: 68 can lack any of the antigen regions of SEQ ID NO: 68. Exemplary polypeptide construct variants containing fewer antigen regions as compared to SEQ ID NO: 68 include, but are not limited to, SEQ ID NO: 137 (Antigen region order: HPV E2, HPV11 E7, HPV E4, HPV11 E6, HPV6 E7, HPV6 E6, HPV E4, HPV11 E6, HPV E7, and HPV E6), SEQ ID NO: 138 (Antigen region order: HPV E4, HPV11 E6, HPV6 E7, HPV E4, HPV11 E6, HPV E7, HPV11 E7, HPV E6, HPV E2, and HPV11 E7), and SEQ ID NO: 139 (Antigen region order: HPV E7, HPV11 E7, HPV E6, HPV11 E6, HPV6 E7, HPV6 E6, HPV11 E6, HPV E2, HPV11 E7). In certain embodiments, a polypeptide construct variant has more antigen regions as compared to SEQ ID NO: 68. For example, a polypeptide construct variant has 1, 2, 3, 4, 5, 6, 7, 8, 9, or 10 more antigen regions compared to SEQ ID NO: 68. A polypeptide construct variant containing more antigen regions compared to SEQ ID NO: 68 can have one or more antigen regions added to any part of the antigen region section of the polypeptide construct (e.g., the N-terminus, the C-terminus, in between other antigen regions, interrupting other antigen regions, and combinations thereof). Exemplary polypeptide construct variants containing more antigen regions as compared to SEQ ID NO: 68 include, but are not limited to, SEQ ID NO: 140 (Antigen region order: HPV E2, HPV11 E7, HPV E4, HPV11 E6, HPV6 E7, HPV6 E6, HPV E4, HPV11 E6, HPV E7, HPV11 E7, HPV E6, and HPV11 E6), SEQ ID NO: 141 (Antigen region order: HPV E6 E7, HPV E7, HPV11 E7, HPV E6, HPV E4, HPV11 E6, HPV6 E7, HPV6 E6, HPV E4, HPV11 E6, HPV E2, HPV11 E7), and SEQ ID NO: 142 (Antigen region order: HPV E2, HPV11 E7, HPV E4, HPV11 E6, HPV6 E7, HPV E4, HPV6 E6, HPV E4, HPV11 E6, HPV E7, HPV11 E7, HPV E7, HPV E6).

In some embodiments, the antigen region or fusion protein can also include any of the linker peptides described herein (e.g., a rigid linker polypeptide, a flexible linker polypeptide, or a combination thereof).

In some embodiments, the antigen region or fusion protein can also include any of the agonist peptides described herein (also referred to an enhancer agonist peptides or agonist enhancer). An agonist peptide is a modified version of an immunogenic epitope that enhances the immune response. For example, an agonist peptide can improve the recognition of T cells while maintaining compatibility with the native peptide-MHC interaction on tumor cells. See, for example, Tsang, et al., Vaccine 35(19):2605-2611 (2017). Exemplary agonist peptides include, but are not limited to, an HPV6 agonist peptide (e.g., an HPV6 E2 agonist peptide, an HPV6 E4 agonist peptide, an HPV6 E6 agonist peptide, and an HPV E7 agonist peptide), an HPV11 agonist peptide (e.g., an HPV11 E6 agonist peptide and an HPV11 E7 agonist peptide), and an HVP16 agonist peptide. In some embodiments, the agonist peptide, if included, is an HPV16 E6 agonist peptide, for example one comprising the amino acid sequence of SEQ ID NO: 48 or SEQ ID NO: 50. In some embodiments, the agonist peptide, if included, is an HPV16 E7 agonist peptide, for example one comprising the amino acid sequence of SEQ ID NO: 52 or SEQ ID NO: 54.

### IV. Vaccines

The present disclosure provides vaccines comprising the polynucleotides encoding the fusion proteins described herein. In some embodiments, the vaccine comprises a polynucleotide encoding a fusion protein comprising an HPV6 protein selected from an HPV6 E2 protein, an HPV6 E4 protein, an HPV6 E6 protein, and an HPV6 E7 protein; and an HPV11 protein selected from an HPV11 E6 and an HPV11 E7 protein. The vaccines of the present disclosure can be used to prevent and/or treat HPV infection and HPV-associated diseases.

In some embodiments, the vaccine comprises a polynucleotide encoding a fusion protein comprising an HPV6 E2 protein, an HPV6 E4 protein, an HPV6 E6 protein, an HPV6 E7 protein, an HPV11 E6 protein, and an HPV11 E7 protein. In some embodiments, the HPV6 E2 protein comprises the amino acid sequence of SEQ ID NO: 1, the HPV6 E4 protein comprises the amino acid sequence of SEQ ID NO: 3 or 7, the HPV6 E6 protein comprises the amino acid sequence of SEQ ID NO: 11 or 40, the HPV6 E7 protein comprises the amino acid sequence of SEQ ID NO: 5 or 9, the HPV11 E6 protein comprises the amino acid sequence of SEQ ID NO: 42, and the HPV11 E7 protein comprises the amino acid sequence of SEQ ID NO: 45. In some embodiments, the fusion protein comprises an amino acid sequence having at least 80%, 90%, 95%, 97%, 98%, or 99% identity with SEQ ID NO: 68, or comprises the amino acid sequence of SEQ ID NO: 68 or a conservatively-substituted variant thereof. In some embodiments, the fusion protein comprises an amino acid sequence having at least 80% identity with SEQ ID NO: 66, 70, 72, or 74. In some embodiments, the fusion protein further comprises a rigid linker polypeptide and/or an HPV16 E6 or E7 agonist enhancer.

The polynucleotides of the vaccines can be operably linked to elements that facilitate expression of the fusion proteins, such as a promoter, a 5′ untranslated region (UTR), a transcription start site (TSS), a 3′ UTR, a tetracycline responsive element, and/or a kozak region. In some embodiments, the promoter is operably linked to a promoter enhancer region. The vaccines of the present disclosure can be administered by any suitable route, including, for example, intramuscular, subcutaneous, intradermal, intravenous, intraperitoneal, intranasal, oral, or transdermal administration. The vaccines can be administered as a single dose or as multiple doses over time. The vaccines can be administered alone or in combination with other therapeutic agents, such as chemotherapeutic agents, immunomodulatory agents, or other vaccines.

In some embodiments, the vaccine comprises a vector comprising the polynucleotide encoding the fusion protein. The vector can be any suitable vector, such as a plasmid, a viral vector, a bacterial vector, or a yeast vector. In some embodiments, the vector is a plasmid vector, such as a DNA plasmid vector. In some embodiments, the vector is a viral vector, such as an adenoviral vector, an adeno-associated viral vector, a retroviral vector, a lentiviral vector, a vaccinia viral vector, or a herpes viral vector. In some embodiments, the vector is an adenoviral vector, such as a gorilla adenoviral vector.

In some embodiments, the vaccine comprises a polypeptide encoded by the polynucleotide. The polypeptide can be produced by any suitable method, such as expression in a host cell, expression in a cell-free system, or chemical synthesis. The polypeptide can be purified by any suitable method, such as affinity chromatography, ion exchange chromatography, size exclusion chromatography, or high-performance liquid chromatography (HPLC). The polypeptide can be formulated with a pharmaceutically acceptable carrier for administration as a vaccine.

In some embodiments, the vaccine comprises a composition comprising the polynucleotide, vector, or polypeptide. The composition can further comprise additional components, such as adjuvants, stabilizers, preservatives, or other therapeutic agents. Suitable adjuvants include, for example, aluminum salts, oil-in-water emulsions, toll-like receptor (TLR) agonists, and saponins. The choice of adjuvant can depend on factors such as the desired immune response, the route of administration, and the target population.

In some embodiments, the vaccine comprises a cell comprising the polynucleotide, vector, polypeptide, or composition. The cell can be any suitable cell, such as a bacterial cell, a yeast cell, an insect cell, or a mammalian cell. In some embodiments, the cell is an immune cell, such as a dendritic cell, a macrophage, a monocyte, a B cell, a T cell, or a natural killer (NK) cell. In some embodiments, the cell is a dendritic cell, such as a human dendritic cell. In some embodiments, the cell is a T cell, such as a CD4+ T cell or a CD8+ T cell. The T cell can be a naïve T cell, an effector T cell, or a memory T cell. The T cell can be a regulatory T cell (Treg) or a gamma delta T cell. The cell can be autologous or allogeneic to the subject being vaccinated. The cell can be modified to express the fusion protein by any suitable method, such as transfection, transduction, or electroporation. The cell can be administered as a vaccine by any suitable route, such as intravenous, intradermal, or subcutaneous administration. The use of immune cells, such as dendritic cells or T cells, as vaccines can enhance the immune response against the HPV antigens and improve the efficacy of the vaccine.

### V. Methods of Treatment

The present invention relates in part to a method of treating a disease or disorder in a subject in need thereof, comprising administering to the subject a polynucleotide, polypeptide, vector, composition, vaccine, or cell of the present invention. In certain embodiments, the method involves administering a polynucleotide, polypeptide, vector, composition, vaccine, or cell of the present invention to subjects with anogenital warts, lower genital tract neoplasia (e.g., cervical, vaginal, and vulvar intraepithelial neoplasia), cervical cancer, vulvar cancer, anal cancer, penile cancer, or head and neck cancers. In certain embodiments, the method involves administering a polynucleotide, polypeptide, vector, composition, vaccine, or cell of the present invention to subjects with malignancies caused by HPV 6/11.

The present invention also relates in part to a method for priming of T-cell responses against HPV-infected (e.g., HPV 6/11+) cells in a subject in need thereof (e.g., a subject with RRP), the method comprising administering to the subject the vector of the present invention. In certain embodiments, the method involves the administration of a polynucleotide, polypeptide, vector, composition, vaccine, or cell of the present invention to subjects with malignancies caused by HPV 6/11.

The present invention also relates to inducing an anti-HPV immune response in a subject in need thereof, such as those with RRP or other HPV-associated diseases or disorders, including those associated with HPV6 or HPV11. Inducing this immune response can involve increasing the recruitment, quantity, or proliferation of various immune cells, including but not limited to dendritic cells, Langerhans cells, natural killer cells, natural killer T cells, and keratinocytes, compared to an HPV immune response without the administration of the described polynucleotides, vectors, fusion proteins, or compositions. In some cases, inducing an anti-HPV immune response includes administering to the subject a therapeutically effective amount of any of the polynucleotides, vectors, fusion protein, or compositions thereof described herein. In some embodiments, inducing an anti-HPV immune response includes administering to the subject a therapeutically effective amount of any of the vectors described herein (e.g., a vector including SEQ ID NO: 68). In some embodiments, the therapeutically effective amount of the vector comprises about 1×10¹¹ and about 5×10¹¹ particle units (PU).

In certain embodiments, the disease or disorder to be treated is RRP and the route of administration is subcutaneously.

In certain embodiments, the method of the invention protects against disease progression with a lower PU dose than previous methods known in the art. For example, in some embodiments the method protects against disease protection with a 5e⁹ PU dose of the vector, composition, or vaccine. In other embodiments the method protects against disease protection with a 5e10 PU dose of the vector, composition, or vaccine. In some embodiments, the method of the invention protects against disease progression with fewer administrations of the therapeutic composition than previous methods known in the art. For example, in some embodiments the method protects against disease protection with only a single administration of the vector, composition, or vaccine.

In certain embodiments, the subject being treated is a mammal, for example, a primate. In some embodiments, the subject being treated is a human.

The method may involve the administration of the polynucleotide, polypeptide, vector, composition, vaccine, or cell in an amount therapeutically effective to treat the disease or disorder. The method may involve the administration of the polynucleotide, polypeptide, vector, composition, vaccine, or cell in an amount therapeutically effective to increase the activity of T-cell responses against specific HPV proteins or antigens (e.g., HPV6/11-specific proteins or antigens). The method may involve the administration of the polynucleotide, polypeptide, vector, composition, vaccine, or cell in an amount therapeutically effective to treat RRP. The method may involve the administration of the polynucleotide, polypeptide, vector, composition, vaccine, or cell in an amount therapeutically effective to decrease a subject's Derkay score.

The effective amount may vary depending on the subject's condition, age, gender, medical history, and/or weight. The amount may also vary depending on the condition to be treated, the anti-inflammatory agent encoded, the type of vector, cell, and/or vaccine used for administration, and the route of administration.

In certain embodiments, the vector, composition, or vaccine is administered in doses. In certain embodiments, the dosage amount in a dose may comprise about 0.1×10⁹ to about 10×10¹² particle units, 0.1×10⁹ to about 1.0×10¹² particle units, about 0.1×10⁹ to about 10×10¹¹ particle units, about 0.1×10⁹ to about 1.0×10¹¹ particle units, about 0.5×10⁹ to about 0.5×10¹¹ particle units, about 0.5×10⁹ to about 0.1×10¹¹ particle units, about 1.0×10¹⁰ to about 10×10¹¹ particle units, about 1.0×10¹⁰ to about 0.1×10¹¹ particle units, about 0.1×10¹¹ to about 10×10¹¹ particle units, about 0.5×10¹¹ to about 9×10¹¹ particle units, about 0.5×10¹¹ to about 8×10¹¹ particle units, about 0.5×10¹¹ to about 7×10¹¹ particle units, about 0.5×10¹¹ to about 6×10¹¹ particle units, about 0.5×10¹² to about 10×10¹² particle units, about 0.5×10¹² to about 1.0×10¹² particle units, about 1.0×10¹¹ to about 0.1×10¹² particle units, about 0.1×10¹² to about 10×10¹² particle units, about 1×10¹⁰ particle units, about 5×10¹⁰ particle units, about 5×10¹¹ particle units, about 6×10¹¹ particle units, about 7×10¹¹ particle units, about 8×10¹¹ particle units, about 9×10¹¹ particle units, about 10×10¹¹ particle units, about 1×10¹² particle units, about 2×10¹² particle units, about 3×10¹² particle units, about 4×10¹² particle units, about 5×10¹² particle units, about 6×10¹² particle units, about 7×10¹² particle units, about 8×10¹² particle units, about 9×10¹² particle units, or about 10×10¹² particle units.

In certain embodiments, the dosage amount may comprise about 1.0×10⁵ to about 1.0×10¹⁰ plaque forming units (PFU), for example, about 0.5×10⁵ to about 0.5×10¹⁰ PFU, about 0.1×10⁵×0.1×10¹⁰ PFU, about 1×10⁶ to about 1×10⁹ PFU, about 0.5×10⁶ to about 0.5×10⁹ PFU, about 0.1×10⁶ to about 0.1×10⁹ PFU, about 1×10⁷ to about 1×10⁸ PFU, about 0.5×10⁷ to about 0.5×10⁸ PFU, about 0.1×10⁷ to about 0.1×10⁸ PFU, about 1.0×10⁶ to about 1.0×10⁹ PFU, about 0.5×10⁶ to about 0.5×10⁹ PFU, about 1.0×10⁷ to about 1×10⁸ PFU, about 1.0×10⁶ to about 1.0×10⁸ PFU, about 0.5×10⁶ to about 0.5×10⁸ PFU, or about 0.1×10⁶ to about 0.1×10⁸ PFU.

In some embodiments, the viral vector may be quantified by Quantitative PCT Analysis (Q-PCR) or analytical HPLC.

For the treatment of HPV-associated pathologies, a dose of the vector may, for example, be about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

For the treatment of RRP, a dose of the vector may, for example, be about 0.1×10⁹ to about 10×10¹² viral particles, about 0.5×10⁹ to about 9×10¹² viral particles, about 0.5×10⁹ to about 8×10¹² viral particles, about 0.5×10⁹ to about 7×10¹² viral particles, about 0.5×10⁹ to about 6×10¹² viral particles, about 0.5×10⁹ viral particles to about 5×10¹⁰ viral particles, about 0.1×10¹⁰ viral particles to about 10×10¹¹ viral particles, about 0.5×10¹⁰ to about 9×10¹¹ viral particles, about 0.5×10¹⁰ to about 8×10¹¹ viral particles, about 0.5×10¹⁰ to about 7×10¹¹ viral particles, about 0.5×10¹⁰ to about 6×10¹¹ viral particles, about 0.5×10¹⁰ viral particles to about 5×10¹¹ viral particles, about 0.1×10¹¹ to about 10×10¹¹ viral particles, about 0.5×10¹¹ to about 9.0×10¹¹ viral particles, about 0.5×10¹¹ to about 8.0×10¹¹ viral particles, about 0.5×10¹¹ to about 7.0×10¹¹ viral particles, or about 0.5×10¹¹ to about 6.0×10¹¹ viral particles. A dose of the vecor may, for example, be about 0.1×10¹¹ virus particles, about 0.2×10¹¹ virus particles, about 0.3×10¹¹ virus particles, about 0.4×10¹¹ virus particles, about 0.5×10¹¹ virus particles, about 0.6×10¹¹ virus particles, about 0.7×10¹¹ virus particles, about 0.8×10¹¹ virus particles, about 0.9×10¹¹ virus particles, about 1.0×10¹¹ virus particles, about 0.1×10¹⁰ virus particles, about 0.2×10¹⁰ virus particles, about 0.3×10¹⁰ virus particles, about 0.4×10¹⁰ virus particles, about 0.5×10¹⁰ virus particles, about 0.6×10¹⁰ virus particles, about 0.7×10¹⁰ virus particles, about 0.8×10¹⁰ virus particles, about 0.9×10¹⁰ virus particles, about 1.0×10¹⁰ virus particles, about 0.1×10⁹ virus particles, about 0.2×10⁹ virus particles, about 0.3×10⁹ virus particles, about 0.4×10⁹ virus particles, about 0.5×10⁹ virus particles, about 0.6×10⁹ virus particles, about 0.7×10⁹ virus particles, about 0.8×10⁹ virus particles, about 0.9×10⁹ virus particles, or about 1.0×10⁹ virus particles.

The dose may be adjusted during the course of treatment, for example, after the levels of expression of the transgene are monitored. If the levels are higher or lower than desired, the amount or frequency of the dose may be adjusted accordingly.

The specific initial and continuing dosage regimen for each patient will vary according to the nature and severity of the condition as determined by the attending diagnostician, the activity of the therapeutic agent, the age of the patient, the diet of the patient, time of administration, route of administration, rate of excretion of the drug, drug combinations, and the like.

The desired mode of treatment, number of doses, routes of administration, and dose schedules may be ascertained and/or adjusted in accordance with methodologies known in the art.

The method disclosed herein contemplates the use of any administration route known in the art for delivery of a polynucleotide, polypeptide, vector, composition, vaccine, or cell. For example, administration may be oral, subcutaneous, intramuscular, intravenous, intracranial, intra-articular, intradermal, or transdermal. In certain embodiments, the subcutaneous or intra-articular administration is by way of a syringe. In some such embodiments, the dosage amount of the vector is contained in a composition in the form of an injectable formulation.

In certain embodiments, the dosage amount is contained in a composition having a volume of about 0.1 to about 5 ml, about 0.1 to about 4 ml, about 0.1 to about 3 ml, about 0.1 to about 2 ml, about 0.25 to about 1.75 ml, about 0.5 to about 1.5 ml, about 0.75 to about 1.25 ml, or about 1.0 ml. In some embodiments, the dosage amount is contained in a composition having a volume of about 0.1 ml, about 0.2 ml, about 0.3 ml, about 0.4 ml, about 0.5 ml, about 0.6 ml, about 0.7 ml, about 0.8 ml, about 0.9 ml, about 1.0 ml, about 1.2 ml, about 1.3 ml, about 1.4 ml, about 1.5 ml, about 1.6 ml, about 1.7 ml, about 1.8 ml, about 1.9 ml, about 2.0 ml, about 2.1 ml, about 2.2 ml, about 2.3 ml, about 2.4 ml, about 2.5 ml, about 2.6 ml, about 2.7 ml, about 2.8 ml, about 2.9 ml, about 3.0 ml, about 3.1 ml, about 3.2 ml, about 3.3 ml, about 3.4 ml, about 3.5 ml, about 3.6 ml, about 3.7 ml, about 3.8 ml, about 3.9 ml, about 4.0 ml, about 4.1 ml, about 4.2 ml, about 4.3 ml, about 4.4 ml, about 4.5 ml, about 4.6 ml, about 4.7 ml, about 4.8 ml, about 4.9 ml, or about 5.0 ml.

Administration of the polynucleotide, polypeptide, vector, composition or vaccine may be at any suitable site on the subject. The choice of administration site will vary depending on factors such as the volume of the dose to be administered, the subject's age, the subject's sex, and the type of active agent to be administered. Subcutaneous administration may, for example, be to the subject's limbs, buttocks, or abdomen. For doses having larger volumes, intramuscular administration is preferred. Such may be, for example, to the subject's deltoid, vastus lateralis, ventrogluteal, or dorsogluteal muscle. Intravenous administration may, for example, be to the subject's arm (e.g. at the bend of the arm), the back of the subject's hand, or the top of the subject's foot. Intra-articular administration may, for example, be to the subject's knee, hip, shoulder, or ankle.

The dosing regimen will vary depending on the subject's age, the subject's sex, and the type of active agent to be administered. The dose may be administered hourly, daily, weekly, monthly, or annually.

In certain embodiments, the doses are delivered at intervals at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days apart. In certain embodiments, the doses are delivered at intervals of about twice per day, about once every day, about twice per week, about once every week, about once every two weeks, about once every three weeks, about once every four weeks, or about once every five weeks. In certain embodiments, the second dose is administered about one week after the first dose, about two weeks after the first dose, about three weeks after the first dose, about four weeks after the first dose, or about five weeks after the first dose; the third dose is administered two weeks after the second dose, about three weeks after the second dose, about four weeks after the second dose, about five weeks after the second dose, or about six weeks after the second dose; and the fourth dose is administered about three weeks after the third dose, about four weeks after the third dose, about five weeks after the third dose, about six weeks after the third dose, about seven weeks after the third dose, about eight weeks after the third dose, about nine weeks after the third dose, about ten weeks after the third dose, about eleven weeks after the third dose, or about twelve weeks after the third dose. In one embodiment, the second dose is administered about two weeks after the first dose, the third dose is administered about six weeks after the second dose, and the fourth dose is administered about twelve weeks after the third dose.

In some embodiments, the treatment involves surgical debulking, usually by means of debridement, angiolytic laser, cryotherapy, or carbon dioxide laser. In some embodiments, the surgical procedure is performed via microscopic or endoscopic rigid laryngoscopy, for example, using either a laser or microdebrider to remove papillomas. This may be preceded and/or followed by administration of the polynucleotide, polypeptide, vector, composition or vaccine of the present invention (alone or in combination with another therapeutic agent).

In some embodiments, the treatment of a patient with the polynucleotide, polypeptide, vector, vaccine, or cell described herein, or a pharmaceutical composition comprising the same, reduces and/or eliminates the need for repeated surgical debulking.

### VI. Manufacture of a Medicament

The present invention also relates in part to a use of the polynucleotide, polypeptide, vector, vaccine, or cell described herein, or a composition comprising the same, in the manufacture of a medicament for use in treating a disease or disorder in a subject in need thereof. In certain embodiments, the disease or disorder may be a proliferative disease or disorder, such as cancer or RRP (e.g., HPV 6/11 malignancies).

### VII. Combination Therapies

In some embodiments, the compositions and methods of the present invention can be combined with at least one additional active agent or therapy. Such additional therapies include radiation therapy, surgery (e.g., debulking), chemotherapy, gene therapy, DNA therapy, virus therapy, RNA therapy, immunotherapy, bone marrow transplantation, nanotherapy, monoclonal antibody therapy, or a combination of the aforementioned therapies. The additional therapy may be in the form of an adjuvant or neoadjuvant therapy.

In some embodiments, the combination therapy comprises the administration of the polynucleotide, polypeptide, vector, vaccine, or cell described herein, or a composition comprising the same, and the concomitant administration of one or more additional compounds, molecules, compositions, or agents. The present invention also relates in part to a combination therapy comprising the administration of the polynucleotide, polypeptide, vector, vaccine, or cell described herein, or a composition comprising the same, and the concomitant use of a surgical or non-surgical procedure.

### A. Administration of Combination Therapies

In certain embodiments, the compositions of the present invention may be administered before, during, after, or in various combinations with additional therapy, such as immune checkpoint therapy.

The administration may be made at intervals ranging from simultaneous to minutes to days to weeks. In embodiments where the present composition is provided to the patient separately from the additional therapeutic agent, the operator may generally ensure that no significant time has elapsed between each delivery time, so that the two compositions can continue to exert a beneficial combination effect on the patient. The two therapies may therefore be provided to the patient within about 12 to 24 hours, or 48 hours, or 72 hours of each other, more specifically within about 6 to 12 hours of each other. In some situations, treatment periods are significant over several days (2, 3, 4, 5, 6 or 7 days) to weeks (1, 2, 3, 4, 5, 6, 7 or 8 weeks) between each dose It may be desirable to extend the period.

Various combinations may be used. For the following examples, the composition of the present invention is “A” and the additional therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy to a patient will follow the general protocol for administration of the compound, given the toxicity of the agents, if present. Thus, in some embodiments, there is a step of monitoring toxicity resulting from the combination therapy.

In certain embodiments, the at least one additional therapy comprises the co-administration of an additional agent. In some embodiments, the additional agent may be contained in the same composition that contains the polynucleotide, polypeptide, vector, vaccine, or cell described herein. Such combination therapies may serve to enhance the treatment of a disease or disorder (e.g., improving the subject's response, prolonging the effects of the treatment) and/or to reduce any side-effects of treatment with the anti-inflammatory agent. In some embodiments, HPV 6/11 malignancies are being treated. In some embodiments, RRP is being treated.

Any suitable agent that may be combined with the polynucleotide, polypeptide, vector, vaccine, or cell described herein, or a composition comprising the same, may be used. For example, the agent may be a therapeutic agent, such as a chemotherapy agent, an anti-inflammatory agent, an analgesic, a biological response modifier, a vector comprising such agents, or a cell comprising the therapeutic agent or a nucleic acid encoding the same.

In certain embodiments, the additional agent is administered at or near the same location as the composition comprising the vector of the present invention is administered. In certain other embodiments, the additional agent is administered at a different location, for example, at the opposite side or extremity.

Administration of the additional agent may be simultaneous with the administration of the composition comprising the vector of the present invention. In certain embodiments, the additional agent is contained in the same formulation as that containing the vector and can be administered with the vector in one unitary dose. In certain other embodiments, the additional agent is not contained in the same formulation but is administered at the same time or within a limited time frame (e.g., a single day, hour, or fraction of an hour) from the administration of the vector.

Alternatively, administration of the additional agent may be sequential in relation to the administration of the vector of the present invention. Such may be preferred in instances where minimizing adverse reactions is desired. In such embodiments, the additional agent may be administered on a schedule in accordance with approved dosing regimens for that agent. Alternatively, the agent may be administered in accordance with a schedule that serves to better maximize the therapeutic effects of the combination therapy, while minimizing adverse reactions.

The timing of administration can be tailored to the specific mechanisms of action and pharmacokinetics of each therapy, maximizing synergistic effects and minimizing overlaps in potential toxicities. Furthermore, the treatment regimen can be adapted based on individual patient response and disease progression, offering flexibility for personalized therapeutic strategies. For example, in embodiments in which an interleukin, for example IL-12, is one of the immunotherapies, its initial administration may precede other agents to prime the immune system for enhanced response, followed by subsequent therapies to amplify and direct the activated immune response. Alternatively, concurrent administration of the interleukin with an immunotherapy can create a synergistic immediate boost in anti-tumor activity, while continued interleukin treatment supports sustained immune engagement. The sequential or concurrent administration of interleukin with other immunotherapies offers a dynamic approach to orchestrating robust and durable anti-tumor immune responses, providing greater therapeutic potential compared to administration of individual immunotherapies or agents alone.

In some embodiments, more than one doses of a first therapy is administered to the subject. In certain embodiments, more than one doses of a second therapy is administered to the subject. In still further embodiments, more than one doses of a third therapy is administered to the subject.

In some embodiments, subsequent doses of the first therapy are administered once every one, two, three or four weeks after the initial dose of the first therapy. In certain embodiments, subsequent doses of the second therapy are administered once every one, two, three or four weeks after the initial dose of the second therapy. In still further embodiments, subsequent doses of the third therapy are administered once every one, two, three or four weeks after the initial dose of the third therapy.

In some embodiments, the initial dose of the first therapy is administered at about 1, 2, 3, 4, 5, 6, 7, 8, 9, 10, 11 or 12 hours prior to the administration of the second therapy. In some embodiments, the initial dose of the first therapy is administered at about 1, 2, 3, 4, 5, 6, 7, 8, 9, 10, 11, 12, 13, or 14 days prior to the administration of the second therapy. In some embodiments, the initial dose of the first therapy is administered at about 2, 3, 4, 5, 6, 7, 8, 9, or 10 weeks prior to the administration of the second therapy. In some embodiments, the initial dose of the first therapy is administered at about 2, 3, 4, 5, or 6 months prior to the administration of the second therapy.

In some embodiments, the initial dose of the first therapy is administered at about 1, 2, 3, 4, 5, 6, 7, 8, 9, 10, 11 or 12 hours after the administration of the second therapy. In some embodiments, the initial dose of the first therapy is administered at about 1, 2, 3, 4, 5, 6, 7, 8, 9, 10, 11, 12, 13, or 14 days after the administration of the second therapy. In some embodiments, the initial dose of the first therapy is administered at about 2, 3, 4, 5, 6, 7, 8, 9, or 10 weeks after the administration of the second therapy. In some embodiments, the initial dose of the first therapy is administered at about 2, 3, 4, 5, or 6 months after the administration of the second therapy.

In some embodiments, the initial dose of the first therapy is administered at about 1, 2, 3, 4, 5, 6, 7, 8, 9, 10, 11 or 12 hours prior to the administration of the third therapy. In some embodiments, the initial dose of the first therapy is administered at about 1, 2, 3, 4, 5, 6, 7, 8, 9, 10, 11, 12, 13, or 14 days prior to the administration of the third therapy. In some embodiments, the initial dose of the first therapy is administered at about 2, 3, 4, 5, 6, 7, 8, 9, or 10 weeks prior to the administration of the third therapy. In some embodiments, the initial dose of the first therapy is administered at about 2, 3, 4, 5, or 6 months prior to the administration of the third therapy.

In some embodiments, the initial dose of the first therapy is administered at about 1, 2, 3, 4, 5, 6, 7, 8, 9, 10, 11 or 12 hours after the administration of the third therapy. In some embodiments, the initial dose of the first therapy is administered at about 1, 2, 3, 4, 5, 6, 7, 8, 9, 10, 11, 12, 13, or 14 days after the administration of the third therapy. In some embodiments, the initial dose of the first therapy is administered at about 2, 3, 4, 5, 6, 7, 8, 9, or 10 weeks after the administration of the third therapy. In some embodiments, the initial dose of the first therapy is administered at about 2, 3, 4, 5, or 6 months after the administration of the third therapy.

In some embodiments, the initial dose of the second therapy is administered at about 1, 2, 3, 4, 5, 6, 7, 8, 9, 10, 11 or 12 hours prior to the administration of the third therapy. In some embodiments, the initial dose of the second therapy is administered at about 1, 2, 3, 4, 5, 6, 7, 8, 9, 10, 11, 12, 13, or 14 days prior to the administration of the third therapy. In some embodiments, the initial dose of the second therapy is administered at about 2, 3, 4, 5, 6, 7, 8, 9, or 10 weeks prior to the administration of the third therapy. In some embodiments, the initial dose of the second therapy is administered at about 2, 3, 4, 5, or 6 months prior to the administration of the third therapy.

In some embodiments, the initial dose of the second therapy is administered at about 1, 2, 3, 4, 5, 6, 7, 8, 9, 10, 11 or 12 hours after the administration of the third therapy. In some embodiments, the initial dose of the second therapy is administered at about 1, 2, 3, 4, 5, 6, 7, 8, 9, 10, 11, 12, 13, or 14 days after the administration of the third therapy. In some embodiments, the initial dose of the second therapy is administered at about 2, 3, 4, 5, 6, 7, 8, 9, or 10 weeks after the administration of the third therapy. In some embodiments, the initial dose of the second therapy is administered at about 2, 3, 4, 5, or 6 months after the administration of the third therapy.

### B. Exemplary Combination Therapies

Anti-inflammatory agents for use in the such combination therapy include: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

Exemplary analgesics for use in combination therapy include acetaminophen, oxycodone, tramadol or proporxyphene hydrochloride.

Exemplary biological response modifiers suitable for use in combination therapy according to the present invention include, for example, molecules directed against cell surface markers (e.g., CD4, CD5, etc.); cytokine inhibitors, such as the TNF inhibitors (e.g., etanercept (ENBREL®), adalimumab (HUMIRA®), and infliximab (REMICADE®); chemokine inhibitors; cell signaling inhibitors, such as EGFR inhibitors (e.g., Gefinitnib (IRESSA®) and Erlotinib (TARCEVA®)), nucleotide analogs (e.g., Cidofovir), angiogenesis inhibitors, such as Bevacizumab (AVASTIN®), non-steroidal anti-inflammatory compounds (NSAIDs), such as COX-2-selective drugs (e.g., Celexecob (CELEBREX®)), immune checkpoint inhibitors, such as PD-1 inhibitors (e.g., Pembrolizumab (KEYTRUDA®), Nivolumab (OPDIVO®), and Cemiplimab (LIBTAYO®) and PD-L1 inhibitors (e.g., Atezolizumab (TECENTRIQ®), Avelumab (BAVENCIO®), and Durvalumab (IMFINZI®), adhesion molecule inhibitors, and other adjuvant therapies. The biological response modifiers include monoclonal antibodies as well as recombinant forms of molecules. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular), and minocycline.

The HPV vaccine antigens of the present invention may be administered in combination with a second therapeutic agent, such as a biological response modifier. In some embodiments, the biological response modifier is administered at a dose ranging from about 0.1 mg/kg to about 10 mg/kg. In particular embodiments, the biological response modifier is administered at a dose of about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 5 mg/kg, or about 10 mg/kg. In a particular embodiment, the biological response modifier is administered at a dose of 2 mg/kg. In another embodiment, the biological response modifier is administered at a dose of 10 mg/kg.

In some embodiments, the biological response modifier is administered at a dose of 10 mg, 50 mg, 100 mg, 150 mg, 200 mg, 250 mg, 300 mg, 350 mg, 400 mg, 450 mg, 500 mg, 550 mg, 600 mg, 650 mg, 700, mg, 750 mg, 800 mg, 850 mg, 900 mg, 950 mg, or 1,000 mg. In a particular embodiment, the biological response modifier is administered at a dose of 200 mg. In another embodiment, the biological response modifier is administered at a dose of 400 mg.

The dosing regimen for the biological response modifier may be adjusted based on individual patient factors, such as body weight, renal function, and liver function. A person of ordinary skill in the art can determine the most appropriate dosing schedule for each patient.

The biological response modifier may be administered prior to, concurrently with, or subsequent to the administration of the HPV vaccine antigens. For example, the biological response modifier may be administered approximately 1 day, 3 days, 1 week, 2 weeks, or 1 month before or after administration of the HPV vaccine antigens.

In some embodiments, the biological response modifier may be administered multiple times, including but not limited to twice a week, once a week, once every two weeks, once every three weeks, once every four weeks, once every five weeks, once every six weeks, once every seven weeks, once every eight weeks, once every nine weeks, or once every ten weeks.

The duration of treatment with the biological response modifier may vary depending on the cancer type, response to therapy, and tolerability. In some cases, treatment may continue until disease progression or unacceptable toxicity, while in others, a fixed duration of treatment (e.g., 1-2 years) may be recommended. In some embodiments, the duration of therapy for the biological response modifier may be up to 12 months, 18 months, 24 months, 30 months, or 36 months. In a particular embodiment, the duration of therapy for the biological response modifier is up to 24 months.

The HPV vaccine antigens of the present invention may be administered in combination with a biological response modifier at the dosing ranges disclosed herein for the treatment of various cancers. Exemplary cancers that may be treated with the combination of HPV vaccine antigens and a biological response modifier include, but are not limited to, cervical cancer, vulvar cancer, vaginal cancer, anal cancer, penile cancer, oropharyngeal cancer (throat cancer), and recurrent respiratory papillomatosis (RRP), melanoma, non-small cell lung cancer (NSCLC), small cell lung cancer (SCLC), head and neck squamous cell carcinoma (HNSCC), classical Hodgkin lymphoma (cHL), primary mediastinal large B-cell lymphoma (PMBCL), urothelial carcinoma, microsatellite instability-high (MSI-H) or mismatch repair deficient (dMMR) solid tumors, gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) solid tumors, cutaneous squamous cell carcinoma (cSCC), and triple-negative breast cancer (TNBC).

In some embodiments of the present invention, the biological response modifier administered in combination with the HPV vaccine antigens is Pembrolizumab (Pembro). Pembro is a humanized monoclonal antibody that blocks the interaction between PD-1 and its ligands, PD-L1 and PD-L2. Pembro may be administered at the dosing ranges disclosed herein, and may be dependent on the cancer being treated and the patient's individual characteristics. In some embodiments, the dosing regimen for Pembro may be: a) 200 mg, administered as an intravenous infusion over 30 minutes every 3 weeks (Q3W); b) 400 mg, administered as an intravenous infusion over 30 minutes every 6 weeks (Q6W); c) 2 mg/kg, administered as an intravenous infusion over 30 minutes every 3 weeks (Q3W); or d) 10 mg/kg, administered as an intravenous infusion over 30 minutes every 2 weeks (Q2W) or every 3 weeks (Q3W).

In particular embodiments, the combination of HPV vaccine antigens and a biological response modifier, such as Pembro, is administered for the treatment of cervical cancer, HPV-related carcinoma, HPV-related malignancy, and/or oropharyngeal squamous cell carcinoma. These cancers are known to be associated with HPV infection, and the combination therapy disclosed herein may provide enhanced therapeutic efficacy compared to either the HPV vaccine antigens or the biological response modifier alone.

In certain embodiments, HPV vaccine antigens provided herein are co-delivered and/or co-expressed (e.g., as part of the same HPV antigen delivery vector or via a separate vector) along with other cytokines. In certain embodiments, HPV vaccine antigens provided herein, are polynucleotides encoding gene-switch polypeptides and a cytokine, or variant or derivative thereof, and methods and systems incorporating the same. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCLI0, CXCLI1, CXCL12, CXCL13, CXCL14, CXCL15, CXCL16, and CXCLI7; the XC subfamily: XCLI and XCL2; and the CX3C subfamily CX3CL1.

In certain embodiments, HPV vaccine antigens provided herein are co-delivered and/or co-expressed along with cyclin-dependent kinase inhibitors (CKIs). In some embodiments, the CKIs specifically inhibit CDK4 and CDK6 (e.g., p16INK4a). In some embodiments, the CKIs consist of one or more 21Cip1, p27Kip1, or p57Kip2. In some embodiments, the CKIs are delivered via administration of Palbociclib (Ibrance), Ribociclib (Kisqali), or Abemaciclib (Verzenio).

In some embodiments, the dose of the CKIs administered is about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg.

In certain embodiments, HPV vaccine antigens provided herein are co-delivered and/or co-expressed (e.g., as part of the same HPV antigen delivery vector or via a separate vector) along with interferons. Interferons (IFNs) comprise interferon type I (e.g. IFN-α, IFN-β, IFN-ε, IFN-κ, and IFN-ω), interferon type II (e.g. IFN-γ), and interferon type 111. In some embodiments, IFN-α is further classified into about 13 subtypes including IFNAI, IFNA2, IFNA4, IFNA5, IFNA6, IFNA7, IFNA8, IFNA10, IFNA13, IFNA14, IFNA16, IFNA17, and IFNA21.

In certain embodiments, HPV vaccine antigens provided herein are co-delivered and/or co-expressed (e.g., as part of the same HPV antigen delivery vector or via a separate vector) along with an interleukin. Interleukins are expressed by leukocytes or white blood cells and promote the development and differentiation of T and B lymphocytes and hematopoietic cells. Exemplary interleukins include IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36, and functional fragments or variants thereof. In some embodiments, interleukins are IL-2, IL-12, IL-15, IL-21, or functional fragment or variants thereof. In some embodiments, the interleukin is IL-15, or a functional fragment or variant thereof, and is comprised in a fusion protein comprising IL-15, or a functional variant thereof, and IL-15a, or a functional fragment or variant thereof.

In certain embodiments, the interleukin is IL-12, or a functional fragment or variant thereof. IL-12 is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as p70), and a homodimer of p40 are formed following protein synthesis. IL-12 is the master regulator of the immune system. IL-12 induces the local and systemic production of IL-12, initiates a cytokine cascade resulting in downstream endogenous interferon-γ (IFN-γ), and via these signaling pathways activates both innate (i.e., NK cells) and adaptive (i.e., cytotoxic T lymphocytes) immunities. The adaptive immune system induces T cells to change from a naïve phenotype to an effector functional type or a memory type. The Th1/Th2 phenotype reflects the result of naïve T cell activation. IL-12 also acts to remodel the tumor microenvironment (TME) and has anti-angiogenic effects. IL-12 binds to the IL-12 receptor (IL-12R), which is a heterodimeric receptor formed by IL-12R-01 and IL-12R-02. The receptor complex is primarily expressed by T cells, but also other lymphocyte subpopulations have been found to be responsive to IL-12.

IL-12 is a candidate for tumor immunotherapy in humans because it functions in bridging innate and adaptive immunity. Indeed, IL-12 has proven effective in animal models of tumor therapy. However, clinically severe side effects were frequently associated with systemic administration of IL-12 in human therapeutic studies. Despite such hurdles, however, IL-12 continues to be of significant interest for use in human (clinical) oncology, particularly because its full therapeutic potential when used by itself or in combination with other onco-therapeutic compounds and methods of treatment, or in particular, via local production rather than systemic administration, has not been fully investigated, much less realized.

In certain embodiments, the IL-12 is a single chain IL-12 (scIL-12), protease sensitive IL-12, destabilized IL-12, membrane bound IL-12, or intercalated IL-12. In some instances, the IL-12 variants are as described in WO2015/095249, WO2016/048903, WO2017/062953.

In certain embodiments, HPV vaccine antigens provided herein are delivered to, and/or are expressed in a subject, in conjunction with delivery and/or expression of IL-12, or a functional fragment or variant thereof. In some embodiments an IL-12 polypeptide, or functional fragment or variant thereof, is expressed from the same HPV vaccine antigen expression vector. In other embodiments, the IL-12 polypeptide, or functional fragment or variant thereof, is expressed from a separate vector in conjunction with HPV vaccine antigen delivery or expression. In some embodiments, the vector expressing IL-12, or a functional fragment or variant thereof, is a replication-deficient adenoviral vector (e.g., a GC46 Gorilla adenovector).

In certain embodiments, in conjunction with delivery or expression of the present invention (i.e., the novel HPV antigen designs disclosed herein), expression of an interleukin in a subject is controlled by constitutive or inducible regulation of expression. In a preferred embodiment, in conjunction with delivery or expression of the present invention, expression of the interleukin in a subject is controlled by inducible regulation of expression (also referred to as, inducibly regulated expression of interleukin).

In certain embodiments, the IL-12 is expressed in a genetic construct comprising a polynucleotide encoding IL12p40, or a functional fragment or variant thereof, linked by way of an IRES (e.g., an EMCV IRES) to a polynucleotide encoding an IL12p35, or a functional fragment or variant thereof. In certain other embodiments, the IL-12 is expressed as a fusion protein comprising an IL12p40, or a functional fragment or variant thereof, and IL12p35, or a functional fragment or variant thereof. In certain such embodiments, the IL12p40, or a functional fragment or variant thereof, is linked by way of a peptide linker with IL12p35, or a functional fragment or variant thereof.

In certain embodiments, in conjunction with delivery or expression of the present invention, IL-12 is expressed as a single chain IL12p70 built into a GC46 Gorilla adenovector (either the same, or separate from, the adenovector delivering the HPV vaccine antigen of the present invention) that has the capability to deliver dose-dependent production of bioactive IL12. In further embodiments, there is no preexisting immunity or presence of neutralizing antibodies directed against the GC46 gorilla adenovector(s) that may limit utility in treating patients with the present invention in combination with IL-12. In certain embodiments, the single chain IL-12p70 has bioactivity similar to that of natural recombinant protein and no propensity of producing the regulatory IL-12p40 homodimer.

In certain embodiments, the interleukin is delivered intratumorally in conjunction with the present invention. In other embodiments, the interleukin is delivered locally to the site of the tumor or to a lymph node associated with the tumor.

In certain embodiments, the vector expressing the interleukin is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹, or 1×10¹², or 2×10¹² viral particles (vp). In some embodiments, the vector is administered at a dose of about 2×10¹¹ vp. In other embodiments, the vector is administered at a dose of about 5×10¹¹ vp.

The initial dose of the composition or vector expressing the novel HPV antigen designs disclosed herein and the initial dose of interleukin is administered concurrently or sequentially. For example, the initial dose of the composition or vector expressing the HPV antigens may be administered at a period of time after the initial dose of interleukin. Alternatively, the initial dose of the composition or vector expressing the HPV antigens may be administered at a period of time before the initial dose of interleukin. In some embodiments, the initial dose of interleukin is administered at about 1, 2, 3, 4, 5, 6, 7 or more days prior to the administration of the composition or vector expressing the HPV antigens. In some embodiments, one or more subsequent doses of interleukin are administered after the administration of the initial dose of the composition or vector expressing the HPV antigens. In some embodiments, one or more subsequent doses of interleukin are administered within 7 to 28 days after the administration of the composition or vector expressing the HPV antigens. In some embodiments, one or more subsequent doses of interleukin are administered at least 7, 8, 9, 10, 11, 12, 13, 14, 15, 16, 17, 18, 19, 20, 21, 22, 23, 24, 25, 26, 27, 28 or more days after administration of the composition or vector expressing the HPV antigens. In some embodiments, one of the subsequent doses of interleukin is administered at 15 days after administration of the composition or vector of expressing the HPV antigens.

In other embodiments, subsequent doses of interleukin are administered once every one, two, three or four weeks after the first dose of interleukin. In further such embodiments, subsequent doses of interleukin are administered once every two weeks or once every four weeks after the first dose of interleukin.

In some embodiments, the interleukin is a membrane-bound IL-15. In certain such embodiments, the membrane-bound IL-15 (mbIL-15) comprises a full-length IL-15 (e.g., a native IL-15 polypeptide) or functional fragment or variant thereof, fused in frame with a full length IL-15Rα, or a functional fragment or variant thereof. In some cases, the IL-15 is indirectly linked to the IL-15Rα through a linker. In some instances, the mbIL-15 is as described in Hurton et al., “Tethered IL-15 augments antitumor activity and promotes a stem-cell memory subset in tumor-specific T cells,” PNAS 2016.

In certain embodiments, HPV vaccine antigens provided herein are co-delivered and/or co-expressed (e.g., as part of the same HPV antigen delivery vector or via a separate vector) along with tumor necrosis factors. Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

In certain embodiments, HPV vaccine antigens provided herein, are co-delivered and/or co-expressed (e.g., as part of the same HPV antigen delivery vector or via a separate vector) along with colony stimulating factors. Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In certain embodiments, HPV vaccine antigens provided herein may be co-delivered and/or co-expressed (e.g., as part of the same HPV antigen delivery vector or via a separate vector) along with surface active agents such as immune-stimulating complexes (ISCOMS). Freunds incomplete adjuvant, LPS analog including monophosphoryl Lipid A (WL), muramyl peptides, quinone analogs and vesicles such as squalene and squalene, and hyaluronic acid may also be used administered in conjunction with the gene construct.

In certain embodiments, an additional element may be added which serves as a target for cell destruction if it is desirable to eliminate cells receiving the genetic construct for any reason. A herpes thymidine kinase (tk) gene in an expressible form can be included in the gene construct. The drug gangcyclovir can be administered to the individual and that drug will cause the selective killing of any cell producing tk, thus, providing the means for the selective destruction of cells with the genetic construct.

In certain embodiments, the additional therapy is administration of a small molecule enzyme inhibitor or anti-metastatic agent. In some embodiments, the additional therapy is the administration of a side effect limiting agent (e.g., an agent intended to lower the incidence and/or severity of side effects of treatment, such as nausea, etc.). In some embodiments, the additional therapy is radiation therapy. In some embodiments, the additional therapy is surgery. In further embodiments, the surgery is debulking surgery. In some embodiments, the additional therapy is a combination of radiation therapy and surgery. In some embodiments, the additional therapy is gamma irradiation.

### 1. Chemotherapy

In some embodiments, the additional therapy is chemotherapy. The additional therapy can be one or more chemotherapeutic agents known in the art, such as dacarbazine or temozolomide. The term “chemotherapy” refers to the use of drugs to treat cancer. “Chemotherapy agent” refers to a compound or composition administered in the treatment of cancer. These agents or drugs are classified according to their mode of activity within the cell, for example whether they affect the cell cycle and at what stage. Alternatively, the agent can be characterized based on its ability to directly cross-link DNA, insert into DNA, or influence nucleic acid synthesis to induce chromosomal and mitotic mutations.

Exemplary chemotherapy agents that may be administered in combination with the compositions of the present invention include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 1311-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). Examples of further chemotherapeutic agents include Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-F-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the chemotherapy agent is a histone decetylase (“HDAC”) inhibitor. HDACs are enzymes involved in the regulation of gene expression by modifying chromatin structure. They remove acetyl groups from histones, leading to chromatin condensation and repression of gene transcription. In cancer, dysregulation of histone acetylation and deacetylation processes can contribute to the development and progression of the disease. HDAC inhibitors represent a promising class of anticancer agents that target epigenetic alterations and dysregulated gene expression in cancer cells. HDAC inhibitors may inhibit angiogenesis, induce apoptosis or cell cycle arrest in cancer cells, and/or promote histone acetylation, which can lead to re-expression of tumor suppressor genes and inhibition of oncogenes. While several HDAC inhibitors have been developed and are undergoing clinical trials, further research is needed to optimize their efficacy and safety profiles for the treatment of various types of cancer. Examples of HDAC inhibitors that may be administered in combination with the compositions of the present invention include, without limitation, vorinostat, romidepsin, belinostat, panobinostat, entinostat, and trichostatin A.

In some embodiments, the chemotherapy agent is a taxoid. For example, the taxoid is docetaxel. In some embodiments, the chemotherapy agent is a platinum coordination complex. For example, the platinum coordination complex is cisplatin. In some embodiments, two chemotherapy agents are used. For example, the two chemotherapy agents are a taxoid and a platinum coordination complex. In some embodiments, the two chemotherapy agents are docetaxel and cisplatin.

In some embodiments, the chemotherapy agent is administered at a dose ranging from about 0.1 mg/kg to about 10 mg/kg. In particular embodiments, the chemotherapy agent is administered at a dose of about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 5 mg/kg, or about 10 mg/kg. In a particular embodiment, the chemotherapy agent is administered at a dose of 2 mg/kg. In another embodiment, the chemotherapy agent is administered at a dose of 3.675 mg/kg. In another embodiment, the chemotherapy agent is administered at a dose of 10 mg/kg.

In some embodiments, the chemotherapy agent is administered at a dose of 10 mg, 50 mg, 75 mg, 100 mg, 142.5 mg, 150 mg, 200 mg, 250 mg, 300 mg, 350 mg, 400 mg, 450 mg, 500 mg, 550 mg, 600 mg, 650 mg, 700, mg, 750 mg, 800 mg, 850 mg, 900 mg, 950 mg, or 1,000 mg. In a particular embodiment, the chemotherapy agent is administered at a dose of 142.5 mg. In a particular embodiment, the chemotherapy agent is administered at a dose of 200 mg. In another embodiment, the chemotherapy agent is administered at a dose of 400 mg.

The dosing regimen for the chemotherapy agent may be adjusted based on individual patient factors, such as body weight, renal function, and liver function. A person of ordinary skill in the art can determine the most appropriate dosing schedule for each patient.

The chemotherapy agent may be administered prior to, concurrently with, or subsequent to the administration of the HPV vaccine antigens. For example, the chemotherapy agent may be administered approximately 1 day, 3 days, 1 week, 2 weeks, or 1 month before or after administration of the HPV vaccine antigens.

In some embodiments, the chemotherapy agent may be administered multiple times, including but not limited to twice a week, once a week, once every two weeks, once every three weeks, once every four weeks, once every five weeks, once every six weeks, once every seven weeks, once every eight weeks, once every nine weeks, or once every ten weeks.

The duration of treatment with the chemotherapy agent may vary depending on the cancer type, response to therapy, and tolerability. In some cases, treatment may continue until disease progression or unacceptable toxicity, while in others, a fixed duration of treatment (e.g., 1-2 years) may be recommended. In some embodiments, the duration of therapy for the chemotherapy agent may be up to 12 months, 18 months, 24 months, 30 months, or 36 months. In a particular embodiment, the duration of therapy for the chemotherapy agent is up to 24 months.

The HPV vaccine antigens of the present invention may be administered in combination with one or more chemotherapy agents at the dosing ranges disclosed herein for the treatment of various cancers. Exemplary cancers that may be treated with the combination of HPV vaccine antigens and a chemotherapy agent include, but are not limited to, cervical cancer, vulvar cancer, vaginal cancer, anal cancer, penile cancer, oropharyngeal cancer (throat cancer), recurrent respiratory papillomatosis (RRP), melanoma, non-small cell lung cancer (NSCLC), small cell lung cancer (SCLC), head and neck squamous cell carcinoma (HNSCC), classical Hodgkin lymphoma (cHL), primary mediastinal large B-cell lymphoma (PMBCL), urothelial carcinoma, microsatellite instability-high (MSI-H) or mismatch repair deficient (dMMR) solid tumors, gastric cancer, esophageal cancer, hepatocellular carcinoma (HCC), Merkel cell carcinoma (MCC), renal cell carcinoma (RCC), endometrial carcinoma, tumor mutational burden-high (TMB-H) solid tumors, cutaneous squamous cell carcinoma (cSCC), and triple-negative breast cancer (TNBC).

In some embodiments of the present invention, the chemotherapy agent administered in combination with the HPV vaccine antigens is docetaxel, cisplatin, or a combination thereof. Docetaxel, cisplatin, or a combination thereof may be administered at the dosing ranges disclosed herein, and may be dependent on the cancer being treated and the patient's individual characteristics. In some embodiments, the dosing regimen for docetaxel may be 142.5 mg (or 75 mg/m²), administered as an intravenous infusion over 60 minutes every 3 weeks (Q3W). In some embodiments, the dosing regimen for cisplatin may be 142.5 mg (or 75 mg/m²), administered as an intravenous infusion over 60 minutes every 3 weeks (Q3W).

In particular embodiments, the combination of HPV vaccine antigens and one or more chemotherapy agents, such as docetaxel or cisplatin, is administered for the treatment of cervical cancer, HPV-related carcinoma, HPV-related malignancy, head and neck cancer, and/or oropharyngeal squamous cell carcinoma. These cancers are known to be associated with HPV infection, and the combination therapy disclosed herein may provide enhanced therapeutic efficacy compared to either the HPV vaccine antigens or the chemotherapy agent(s) alone.

### 2. Radiation Therapy

In certain embodiments, radiation therapy may be used in combination with any of the methods of treatment described herein. “Radiation therapy” refers to treatment for a disease or disorder (typically, cancer) where radioactive energy is used to destroy cells and their division. Modem radiation therapy systems use relatively high energy beams of radiation from radioactive isotopes or electron beam X-Ray or as γ-rays generators. Radiation therapy includes external beam radiation, intensity modulated radiation therapy (IMRT), focused radiation, and any form of radiosurgery including Gamma Knife, Cyberknife, Linac, and interstitial radiation (e.g. implanted radioactive seeds, GliaSite balloon), and/or with surgery. Other forms of DNA damage factors that may be implemented in radiation therapy include microwave, proton beam irradiation (U.S. Pat. Nos. 5,760,395 and 4,870,287) and ultraviolet irradiation The dose range of X-rays ranges from 50 to 200 rotgens per day for a long period of time (3 to 4 weeks) to 2000 to 6000 lentgens for a single dose. The range of irradiation of radioactive isotopes can vary widely, depending on the half-life of the isotope, the intensity and type of radiation emitted, and the rate of absorption of neoplastic cells.

In certain embodiments, radiation therapy may comprise radiation or associated administration of radiopharmaceuticals to a patient is provided. The source of radiation may be either external or internal to the patient being treated (radiation treatment may, for example, be in tie form of external beam radiation therapy (EBRT) or brachytherapy (BT)). Radioactive elements that may, be used in practicing such methods include, e.g., radium, cesium-137, iridium-192, americium-241, gold-198, cobalt-57, copper-67, technetium-99, iodide-123, iodide-131, and indium-1.

### 3. Immunotherapy

In some embodiments, the subject may also be administered an immunotherapeutic agent. “Immunotherapy” refers to a treatment that uses a subject's immune system to treat cancer, e.g., cancer vaccines, cytokines, use of cancer-specific antibodies, T cell therapy, and dendritic cell therapy. In the context of cancer therapy, immunotherapy generally relies on the use of immune effector cells and molecules to target and destroy cancer cells.

In some embodiments, the subject is also administered an immune modulator. An “immune modulator” is a type of drug (large or small molecule, including but not limited to antibodies (immunoglobulins) and other proteins), vaccine or cell therapy which induces, amplifies, attenuates or prevents change in the immune system cells, such as T cells, and some cancer cells. An immune modulator may be, for example, an immune checkpoint inhibitor, a vaccine, a molecule that stimulates T cells and/or NK cells, a cytokine, an antigen specific binder, a T cell, a NK cell, chimeric antigen receptor (CAR) or T-cell receptor (TCR), or a cell expressing a CAR or TCR. Immune modulators may be used to treat cancer, alone or in conjunction with other compounds. Immune modulators include a chemotherapy or a radiation.

Examples of immune modulators include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interleukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1 alpha”), Chemokine (C—C motif) ligand 4 (“MIP-1 beta”), Macrophase inflammatory protein-1-delta (“MIP-1 delta”), Platelet-derived growth factor subunit B (“PDGF-BB”), Chemokine (C—C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“HMR-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP S′1), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-1”), Chemokine (C—C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3 alpha”), C—C motif chemokine 19 (“MIP-3 beta”), Chemokine (C—C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C—C motif) ligand 17 (“TARC”), Thymus-expressed chemokine (“TECK”), Thymic stromal lymphopoietin (“TSLP 4-1BB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), FoUistatin, Follicle-stimulating hormone (“FSH”), Chemokine (C—X—C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD F”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACT”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor k B (“RANK”).

In certain preferred embodiments, the subject is also administered IFN-gamma (IFNγ). In some embodiments, the subject is pretreated with IFNγ, such as with low doses of IFNγ, prior to administering the TCR-modified immune effector cells disclosed herein (e.g., the adoptive immunotherapy compositions disclosed herein comprising the TCR-T cells disclosed herein).

As noted above, in certain embodiments, the immunotherapy can be a cytokine. In some embodiments, the cytokine is a membrane-bound cytokine, which is co-expressed with a chimeric antigen receptor (CAR) described herein. In some instances, the cytokine comprises a chemokine, an interferon, an interleukin, a colony-stimulating factor or a tumor necrosis factor.

In some instances, one or more methods described herein further comprise administration of a cytokine selected from IL2, IL7, IL12, IL15, a fusion of IL-15 and IL-15Rα, IL21, IFNγ or TNF-α.

Non-limiting examples of immune modulators by target type are shown in Table 4.

TABLE 4
Immune Modulators by Target Type

| Category | Target | Examples | Brief Description |
|---|---|---|---|
| Immune | PD-1 inhibitors | Cemiplimab | Block PD-1 protein on T cells, |
| Checkpoint | | Nivolumab | allowing them to attack cancer |
| Inhibitors | | Pembrolizumab | cells more effectively. |
| | | Pidilizumab | |
| | | Spartalizumab | |
| | | AMP-224 | |
| | | AUNP-12 | |
| | | BGB-A317 | |
| | | MEDI-0680 | |
| | | STI-A1110 | |
| | PD-L1 inhibitors | Atezolizumab | Block PD-L1 protein on cancer |
| | | Avelumab | cells, preventing them from |
| | | Durvalumab | hiding from T cells. |
| | | BMS-936559 | |
| | | CK-301 | |
| | | KD033 | |
| | CTLA-4 | Ipilimumab | Block CTLA-4 protein, |
| | inhibitors | Tremelimumab | boosting the immune system's |
| | | | response to cancer. |
| | Negative | VISTA inhibitors | Target VISTA protein, a |
| | checkpoint | | negative regulator of immune |
| | regulators | | response. |
| | Co-inhibitory | Tim-3 inhibitors | Target various co-inhibitory |
| | receptor targets | Tim-3 ligands | receptors and their ligands, |
| | | Galectin 9 antagonists | enhancing immune response. |
| | | CNC225 | |
| | | HMGB1 | |
| | | VB4-845 | |
| | | Phosphatidyl serine | |
| | | CECAM-1 | |
| | | LAG-3 | |
| | | IMP321 | |
| | | TIGIT | |
| | | BMS986207 | |
| | CD25 (IL2RA) | Daclizumab | Targets CD25 receptor, |
| | | | reducing regulatory T cells that |
| | | | suppress immune response. |
| | Toll-like | Anti-TLR2 antibodies | Blocks TLR2 signaling, |
| | Receptors | TLR2 blockade drugs | potentially modulating the |
| | (TLRs) | CU-CPT22 | immune response. |
| | | C29 | |
| | | MMG-11 | |
| Intrinsic | IDO1 | Indoximod | Inhibits IDO1 enzyme, |
| Pathways | | | preventing cancer cells from |
| | | | suppressing immune response. |
| | TDO | CRD1152 | Inhibits TDO enzyme, another |
| | | | player in immune suppression |
| | | | by cancer cells. |
| | Ectonucleotidase | CD39 inhibitors | Block CD39 and CD73 |
| | inhibitors and | CD73 inhibitors | enzymes, involved in regulating |
| | antibodies | OREG-103/BY40 | immune response. |
| | | BMS-986179 | |
| | B7-H3 | MGC018 | Targets B7-H3 protein, |
| | | | potentially blocking its |
| | | | immune-suppressive effects. |
| | Bispecifics | Beta-TRAP (TGF-beta & | Dual-targeting drugs hitting two |
| | | PD-L1) | immune checkpoints |
| | | BMS-936559 (PD-L1 & | simultaneously. |
| | | B7.1) | |
| Co-Stimulatory | Tumor necrosis | GITR agonists | Target various members of the |
| Receptor Targets | factor (TNF) | (INCAGN01876, JNJ- | TNF superfamily involved in |
| | superfamily | 68284767, JNJ-7424) | immune activation. |
| | members | GITR Ligands (AMG594, | |
| | | ALT-836) | |
| | | CD27 agonists | |
| | | (Varlimumab, CDX-1127, | |
| | | SHR-1210) | |
| | | CD137 (4-1BB) agonists | |
| | | (Urelumab, BMS-663516, | |
| | | Utomilumab, PF- | |
| | | 05080825) | |
| | | TNF-alpha Fusion | |
| | | Proteins (AMG 592, | |
| | | REGN3783) | |
| | Co-Stimulatory | OX40 (CD134) agonists | Stimulate co-stimulatory |
| | receptor targets | MEDI6469 (OX40) | receptors on T cells, boosting |
| | | CD40 agonists | their activity. |
| | | Dacetuzumab (CD40) | |
| | | ICP-870,893 (CD40) | |
| | Toll-like | VTX-2337 (TLR8) | Activate TLR8, a receptor |
| | receptor (TLR) | | triggering immune response. |
| | agonists | | |
| | CD28 | BTLA antagonists | Block BTLA, a co-inhibitory |
| | superfamily | | molecule. |
| | costimulatory | | |
| | molecules | | |
| | | ICOS agonists | Activates ICOS, a molecule on |
| | | Vopratelimab (GSK335) | immune cells that plays a |
| | | STI-A1110 | crucial role in their activation |
| | | AT9283 | and survival. |
| | | A-419 | |
| Tumor-Specific | | MAGE-A antigens | Play a crucial role in the |
| Antigens | | NY-ESO-1 | development of cancer |
| | | CEA (carcinoembryonic | immunotherapies, offering |
| | | antigen) | potential targets for the immune |
| | | HER2/neu | system to recognize and destroy |
| | | EGFR (epidermal growth | cancer cells |
| | | factor receptor) | |
| | | Cetuximab (Erbitux) | |
| | | Blinatumomab | |
| | | Catumaxomab | |
| | | Ertumaxomab | |
| Antibody-Drug | | Daratumumab (CD38) | Combine antibody targeting with |
| Conjugates | | Isatuximab (CD38) | for targeted delivery |
| (ADCs) | | Lirilumab (KIR) | |
| | | Elotuzumab (SLAMF-7) | |
| | | IPH2201 (DNAM-1) | |
| | | Arabinoxylan | |
| | | (NKG2A/D) | |
| | | MGN-3 (Nectin-1/2) | |
| Cancer Vaccines | | Dendritic cell vaccine | Train immune system to |
| | | TUMAP vaccine | recognize and attack specific |
| | | Oncofetal antigen vaccine | cancer cells |
| | | Autologous tumor cell | |
| | | lysate vaccine | |
| Viral Vaccines | | HPV (e.g., HPV peptide | Target specific viruses |
| | | vaccines) | associated with cancer |
| | | HBV | development |
| | | Shingles | |
| | | CMV | |
| | | EBV | |
| Immunostimulant | | TLR9 and STING ligands | Enhance immune response to |
| Adjuvants | | | vaccines or other therapies |
| Cellular | Lymphokine- | Metastatic melanoma | Utilize modified immune cells |
| Therapies | activated killer | Renal cell carcinoma | to directly attack cancer cells |
| | (LAK) cells | Other cancers | |
| | Tumor | Melanoma | TILs are immune cells found |
| | infiltrating | Glioblastoma | within tumors and offer a |
| | lymphocytes | | promising personalized |
| | (TILs) | | approach to cancer |
| | | | immunotherapy |
| | Cytokine- | Interleukin-2 (IL-2) | Stimulate and direct the |
| | activated killer | Interferon-alpha (IFN-α) | immune system against cancer |
| | cells | Granulocyte-macrophage | |
| | | colony-stimulating factor | |
| | | (GM-CSF) | |
| | | Cytokine-induced Killer | |
| | | (CIK) cell | |
| | Chimeric antigen | Approved targets (e.g., | Uses genetically modified T |
| | receptor (CAR) | CD19, BCMA) | cells to target and destroy |
| | | B-cell targets (e.g., CD20, | cancer cells |
| | | CD22, CD33, CD38, | |
| | | CD44, CD79b, CD123, | |
| | | FLT3) | |
| | | T-cell targets (CD25, | |
| | | CD4, CD5, CD7, CTLA- | |
| | | 4, PD-1) | |
| | | Myeloid targets (CD33, | |
| | | CD123) | |
| | | Solid tumor targets (e.g., | |
| | | HER2, EGFR, mesothelin, | |
| | | ROR1, CEA, MAGE-A, | |
| | | GD2, PSMA, mesothelin, | |
| | | CD22, EGFR, Folate | |
| | | receptor α, Mucins, etc.) | |
| | T-Cell Receptor | MAGE-A3 | Non-allergenic |
| | (TCR) | NY-ESO-1 | Uses a patient's naturally |
| | | HPV-specific | occurring TCRs, which |
| | | | receptors recognize specific |
| | | | antigens on the surface of cells. |

In some embodiments, one or more of the methods described herein further comprises administering a chimeric antigen receptor (CAR)-T cell therapy, for example the administration of a CAR-T cell. Chimeric receptor therapies, including CAR-T cells, involve the use of cells engineered to express receptors that target specific antigens expressed on tumor cells. Upon binding to a tumor cell, the engineered cell initiates an immune response that results in the destruction of the tumor cell. In some embodiments, the CAR-T cell expresses an antigen that binds to MUC-16, CD33, ROR-1, mesothelin, CD22, CD19, or B Cell Maturation Antigen (BCMA).

An “immune checkpoint inhibitor” is a type of drug (large or small molecule, including but not limited to antibodies (immunoglobulins) and other proteins) which block certain proteins made by some types of immune system cells, such as T cells, and some cancer cells. These proteins help keep immune responses in check and limit or prevent T cells from killing cancer cells. When these proteins are blocked, the molecular “brakes” on the immune system are released and T cells can better (i.e., more effectively) kill cancer cells. Examples of checkpoint proteins found on T cells or cancer cells include PD-1/PD-L1 and CTLA-4/B7-1/B7-2. Immune checkpoint inhibitors may be used to treat cancer; alone or in conjunction with other compounds.

In some of the embodiments of the methods described herein, the immune checkpoint inhibitor is for example, a PD-1 binder, a PD-L1 binder, a CTLA-4 binder, a V-domain immunoglobulin suppressor of T cell activation (VISTA) binder, a TIM-3 binder, a TIM-3 ligand binder, a LAG-3 binder, a T-cell immunoreceptor with Ig and ITIM domains (TIGIT) binder, a B- and T-cell attenuator (BTLA) binder, a B7-H3 binder, a TGFbeta and PD-L1 bispecific binder or a PD-L1 and B7.1 bispecific binder.

In some embodiments, the PD-1 binder is an antibody that specifically binds PD-1. In some embodiments, the PD-1 binder is an antagonist. In some embodiments, the antibody that binds PD-1 is pembrolizumab (KEYTRUDA, MK-3475; CAS #1374853-91-4) developed by Merck, pidilizumab (CT-011; CAS #1036730-42-3) developed by Curetech Ltd., nivolumab (OPDIVO, BMS-936558, MDX-1106; CAS #946414-94-4) developed by Bristol Myer Squibb, MEDIO680 (AMP-514); developed by AstraZenenca/MedImmune, cemiplimab-rwlc (REGN2810, LIBTAYO® CAS #1801342-60-8) developed by Regeneron Pharmaceuticals, BGB-A317 developed by BeiGene Ltd., spartalizumab (PDR-001; CAS #1935694-88-4) developed by Novartis, or STI-A1110 developed by Sorrento Therapeutics. In some embodiments, the antibody that binds PD-1 is described in PCT Publication WO2014/179664, for example, an antibody identified as APE2058, APE1922, APE1923, APE1924, APE 1950, or APE 1963 developed by Anaptysbio, or an antibody containing the CDR regions of any of these antibodies. In other embodiments, the PD-1 binder is a fusion protein that includes the extracellular domain of PD-L1 or PD-L2, for example, AMP-224 (AstraZeneca/MedImmune). In other embodiments, the PD-1 binder is a peptide inhibitor, for example, AUNP-12 developed by Aurigene.

In some embodiments, the PD-L1 binder is an antibody that specifically binds PD-L1. In some embodiments, the PD-L1 binder is an antagonist. In some embodiments, the antibody that binds PD-L1 is atezolizumab (RG7446, MPDL3280A; Tecentriq; CAS #1380723-44-3) developed by Genentech, durvalumab (MEDI4736, IMFINZI® CAS #1428935-60-7) developed by AstraZeneca/MedImmune, BMS-936559 (MDX-1105) developed by Bristol Myers Squibb, avelumab (MSB0010718C; Merck KGaA; Bavencio; CAS #1537032-82-8), KD033 (Kadmon), the antibody portion of KD033, STI-A 1014 (Sorrento Therapeutics) or CK-301 (Checkpoint Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies.

In some embodiments, the CTLA-4 binder is an antibody that specifically binds CTLA-4. In some embodiments, the CTLA-4 binder is an antagonist. In some embodiments, the antibody that binds CTLA-4 is ipilimumab (YERVOY) developed by Bristol Myer Squibb or tremelimumab (CP-675,206) developed by MedImmune/AtraZenica then Pfizer. In some embodiments, the CTLA-4 binder is an antagonistic CTLA-4 fusion protein or soluble CTLA-4 receptor, for example, KAHR-102 developed by Kahr Medical Ltd.

In some embodiments, the 4-1BB (CD137) binder is a binding molecule, such as an anticalin. In some embodiments, the 4-1BB binder is an agonist. In some embodiments, the anticalin is PRS-343 (Pieris AG). In some embodiments, the 4-1BB binder is an agonistic antibody that specifically binds 4-1BB. In some embodiments, antibody that binds 4-1BB is PF-2566 (PF-05082566) developed by Pfizer or urelumab (BMS-663513) developed by Bristol Myer Squibb.

In some embodiments, the LAG3 binder is an antibody that specifically binds LAG3. In some embodiments, the LAG3 binder is an antagonist. In some embodiments, the antibody that binds LAG3 is IMP701 developed by Prima BioMed, IMP731 developed by Prima BioMed/GlaxoSmithKline, BMS-986016 developed by Bristol Myer Squibb, LAG525 developed by Novartis, and GSK2831781 developed by Glaxo SmithKline. In some embodiments, the LAG-3 antagonist includes a soluble LAG-3 receptor, for example, IMP321 developed by Prima BioMed.

In some embodiments, the KIR binder is an antibody that specifically binds KIR. In some embodiments, the KIR binder is an antagonist. In some embodiments, the antibody that binds KIR is lirilumab developed by Bristol Myer Squibb/Innate Pharma.

In some embodiments, a combination of controlled expression of IL-12 with a check point inhibitor, such as but not limited to, a PD-1-specific antibody (e.g., nivolumab) provides improved cancer treatment, such as but not limited to brain cancer (e.g., gliomas/glioblastomas) wherein IL-12 provides therapeutically effective recruitment and infiltration of T cells (such as killer T-cells) into the tumor while the check point inhibitor (e.g., anti-PD-1 antibody) provides for enhanced and/or improved immune cell function and activity within the tumor (i.e., improved anti-tumor immune cell activity).

### 4. Hormone Therapy

In some embodiments, a therapeutic agent for use in combination with a composition of the present invention as described herein may be a hormonal regulating agent (e.g., hormone therapy), such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogen (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

### 5. Surgery

In some embodiments, the compositions of the present invention are administered conjointly with surgery. Therapeutic surgery includes resection in which all or part of a cancer tissue is physically removed, dissected and/or destroyed, and the treatment, chemotherapy, radiotherapy, hormone therapy, gene therapy, immunotherapy and/or alternative therapy of this embodiment. It can be used in conjunction with other therapies. Tumorectomy refers to the physical removal of at least a portion of a tumor. In addition to tumor resection, surgical treatment may include laser surgery, cold surgery, electrosurgery and microscopically controlled surgery. Debulking refers to the reduction of as much of the volume (i.e., bulk) of a tumor without the intention of a complete eradication. Debulking is usually achieved by surgical removal.

Upon incision of some or all cancer cells, tissues or tumors, cavities may form in the body. Treatment can be by perfusion, direct injection, or topical application to the affected area using additional anti-cancer therapies. Such treatment may be, for example, every 1, 2, 3, 4, 5, 6 or 7 days, every 1, 2, 3, 4 or 5 weeks, or every 1, 2, 3, 4, 5, 6, 7, 8, 9, 10, 11 or 12 months. Such treatment can also be achieved in various dosages.

### a. Surgical Options to Treat RRP

In certain embodiments, the method of treatment is combined with a surgical procedure, which may be performed before or after administration of the composition (e.g., a substance comprising a polynucleotide, polypeptide, vector, or vaccine described herein). Indeed, surgery is a common treatment modality for RRP, often augmented by the use of pharmacotherapy as surgery alone usually cannot control the disease. See, e.g., Schraff S, Derkay C S, Burke B, Lawson L. American Society of Pediatric Otolaryngology. Members' experience with recurrent respiratory papillomatosis and the use of adjuvant therapy. Arch Otolaryngol Head Neck Surg 2004; 130(9):1039-1042; Katsenos S, Becker H D. Recurrent respiratory papillomatosis: a rare chronic disease, difficult to treat, with potential to lung cancer transformation: apropos of two cases and a brief literature review. Case Rep Oncol 2011; 4(1):162-171. Surgical treatment consists of, for example, debulking the papillomas to secure a stable airway while preserving the underlying laryngeal tissues. See Venkatesan N N, Pine H S, Underbrink M P. Recurrent respiratory papillomatosis. Otolaryngol Clin North Am. 2012 June; 45(3):671-94.

Surgery is often performed via microscopic or endoscopic rigid laryngoscopy in the operating room using either a laser or microdebrider to remove papillomas. A variety of lasers have been used for this purpose, including CO2, KTP (potassium titanyl phosphate), and pulse-dyed lasers. Although lasers remain the traditional standard, the use of microdebriders has been gaining favor. The reason for this trend is the ability of the microdebrider to selectively suction the affected tissue, which often allows for more precise debridement, limited damage to the underlying tissues, and greater preservation of normal epithelium. These characteristics become increasingly important because of the nature of RRP, which often necessitates several visits to the operating room for debulking, with an average of 4.1 to 4.4 debulking surgeries during the first year of diagnosis alone. See Armstrong L R, Derkay C S, Reeves W C. Initial results from the national registry for juvenile-onset recurrent respiratory papillomatosis. RRP Task Force. Arch Otolaryngol Head Neck Surg. 1999; 125(7):743-8.

One exemplary repeated surgical debulking method consists of using a carbon dioxide (CO2) laser to achieve clearance by using high-temperature vaporization for exophytic and endophytic tumor debulking where suction diathermy is used to control bleeding if hemorrhage is encountered. Coblation is then used in both exophytic and endophytic tumor volumes by using low-temperature radiofrequency and saline to create a plasma field that dissolves tumor tissue. Debulking for endophytic tumors is performed 1-2 mm anterior to the vocal process, carrying laterally through the width of the tumor including the vocalis muscle on the obstructing side. Debulking is continued until an adequate airway is obtained. See, e.g., Gul F, Teleke Y C, Yalciner G, Babademez M A. Debulking obstructing laryngeal cancers to avoid tracheotomy. Braz J Otorhinolaryngol. 2021 January-February; 87(1):74-79.

Among surgical options, a tracheotomy may be performed, but it is typically reserved for those most aggressive cases with impending airway compromise. Although this procedure may be necessary to secure an airway, it does provide another site for rapid colonization and serves as a conduit for disease spread to the tracheobronchial tree. In a series studying patients with RRP in whom a tracheotomy was performed, tracheal papillomas were present in more than half of those patients. Cole R R, Myer C M, III, Cotton R T. Tracheotomy in children with recurrent respiratory papillomatosis. Head Neck. 1989; 11(3):226-30. It is thus widely accepted that tracheotomy should be reserved for only those cases in which multiple debulking surgeries have failed and/or the child's airway becomes compromised. Furthermore, if a tracheotomy is unavoidable, decannulation should be considered as early as possible once the disease process is controlled and the airway is deemed stable.

In some embodiments, a debulking procedure is performed before the first dose is administered. In some embodiments, one or more debulking procedures is performed 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months before the first dose is administered. In one embodiment, three debulking procedures are performed before the first dose is administered. In another embodiment, one or more debulking procedures are performed 12 months before the first dose is administered. In a further embodiment, three debulking procedures are performed 12 months before the first dose is administered.

In some embodiments, one or more debulking procedures is performed after the first dose is administered and before the final dose is administered. In some embodiments, one or more debulking procedures is performed after the final dose is administered. In some embodiments, one or more debulking procedures is performed during the administration of at least one dose of the composition.

In one embodiment, a patient may receive at least one debulking procedure at least 6 months, 1, 2, 3, 4, or 5 years following administration of a dose of the composition described herein, or in a time frame determined by the patient's physician, where the patient may then, subsequent to such a debulking procedure, proceed to receive one or more additional doses of the composition. In some embodiments, clinical efficacy of the therapeutic method is measured after the final dose is administered.

In some embodiments, administration of the vector described herein results in a reduced need for surgical intervention (e.g., debulking surgery). In some embodiments, administration of the vector described herein results in a reduced need for surgical intervention for a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months. In some embodiments, administration of the vector described herein results in a reduced need for surgical intervention for a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 years.

### C. Combination Adjuvant Therapies for RRP

An embodiment of the present invention relates to the use of compositions described herein as therapeutic vaccines against HPV6 and/or HPV11 (HPV6/11) induced or associated diseases; such as the treatment of recurrent respiratory papillomatosis (RRP) embodiment.

Current adjuvant therapies for RRP have a range of actions, including immunomodulation, disruption of HPV replication, control of inflammation, and prevention of angiogenesis. In addition, some of these therapies have only been evaluated in small group or case studies and need more powerful randomized controlled trials to sufficiently evaluate their efficacy in RRP management. Any of these therapies may be used in conjunction with the method of treatment described herein.

### a. Interferon

Interferon (IFN) therapy is one of the first systemic adjuvant treatments used to manage RRP. Broadhurst M S, Akst L M, Burns J A, et al. Effects of 532 nm pulsed-KTP laser parameters on vessel ablation in the avian chorioallantoic membrane: implications for vocal fold mucosa. Laryngoscope 2007; 117(2):220-225. The clinical efficacy of IFN therapy in the treatment of RRP is unsettled. One group reported that 117 of 160 (73.1%) of patients treated with adjuvant IFN-alpha-2b had complete or partial response measured by extent of recurrence. Nodarse-Cuni H, Iznaga-Marin N, Viera-Alvarez D, et al. Interferon alpha-2b as adjuvant treatment of recurrent respiratory papillomatosis in Cuba: National Programme (1994-1999 report). J Laryngol Otol 2004; 118(9):681-687. Conversely, another group showed that initial growth rate reduction of papillomas from IFN-alpha treatment in the first six months post-treatment was not durable and became insignificant in the second six months post-treatment. Healy G B, Gelber R D, Trowbridge A L, Grundfast K M, Ruben R J, Price K N. Treatment of recurrent respiratory papillomatosis with human leukocyte interferon. Results of a multicenter randomized clinical trial. N Engl J Med 1988; 319(7):401-407. Unmodified recombinant IFN-alpha is no longer on the market and has been replaced by pegylated-IFN-alpha-2a (peg-IFN-alpha-2a). One study treated 11 AO-RRP patients with peg-IFN-alpha-2a in combination with granulocyte monocyte-colony-stimulating factor (GM-CSF) and found that 11/11 (100%) showed no relapse at 12 months' follow-up. Suter-Montano T, Montano E, Martinez C, Plascencia T, Sepulveda M T, Rodriguez M. Adult recurrent respirator papillomatosis: a new therapeutic approach with pegylated interferon alpha 2a (Peg-IFNalpha-2a) and GM-CSF. Otolaryngol Head Neck Surg 2013; 148(2):253-260. Side effects for IFN therapy include neurologic disorders, mental disturbances, thrombocytopenia, leukopenia, hair loss, and fever. Gerein V, Rastorguev E, Gerein J, Jecker P, Pfister H. Use of interferon-alpha in recurrent respiratory papillomatosis: 20-year follow-up. Ann Otol Rhinol Laryngol 2005; 114(6):463-471. Despite some positive evidence for adjuvant IFN therapy, it is seldom used due to the emergence of intralesional adjuvants, such as cidofovir and bevacizumab, which have fewer local and systemic side effects.

### b. Cidofovir

Cidofovir is a cytosine nucleotide analog that blocks the replication of DNA viruses by inhibiting viral DNA polymerase. De Clercq E, Descamps J, De Somer P, Holy A. (S)-9-(2,3-Dihydroxypropyl)adenine: an aliphatic nucleoside analog with broad-spectrum antiviral activity. Science. 1978; 200(4341):563-565. Its mechanism of action against HPV is not well-understood, although it has been hypothesized that it acts by augmenting the immune system or inducing apoptosis. Shehab N, Sweet B V, Hogikyan N D. Cidofovir for the treatment of recurrent respiratory papillomatosis: a review of the literature. Pharmacotherapy 2005; 25(7):977-989. Intralesional administration of cidofovir has been fairly well-tolerated, with limited systemic toxicity. See Broekema F I, Dikkers F G. Side-effects of cidofovir in the treatment of recurrent respiratory papillomatosis. Eur Arch Otorhinolaryngol 2008; 265(8):871-879; Dikkers F G. Treatment of recurrent respiratory papillomatosis with microsurgery in combination with intralesional cidofovir-a prospective study. Eur Arch Otorhinolaryngol 2006; 263(5):440-443; Weiner R D, Lee J H, Hoffman H T, Robinson R A, Smith R J H. Case of progressive dysplasia concomitant with intralesional cidofovir administration for recurrent respiratory papillomatosis. Annals of Otology Rhinology and Laryngology 2005; 114(11):836-839. Pudszuhn A, Welzel C, Bloching M, Neumann K. Intralesional Cidofovir application in recurrent laryngeal papillomatosis. Eur Arch Otorhinolaryngol 2007; 264(1):63-70; Snoeck R, Wellens W, Desloovere C, et al. Treatment of severe laryngeal papillomatosis with intralesional injections of cidofovir [(S)-1-(3-hydroxy-2-phosphonylmethoxypropyl)cytosine]. J Med Virol 1998; 54(3):219-225. Prospective trials in patients treated with intralesional cidofovir have shown marked papilloma regression as well as complete disease remission in both the J O—RRP and A O—RRP populations. See Ivancic R, Iqbal H, deSilva B, Pan Q, Matrka L. Current and future management of recurrent respiratory papillomatosis. Laryngoscope Investig Otolaryngol. 2018 Jan. 14; 3(1):22-34, Table 2.

A group investigated the efficacy of intralesional cidofovir following surgical excision in 16 JO-RRP patients and found a 75% complete remission rate, with stable disease to a mean of 33.6 months. In a separate cohort by the same group, intralesional cidofovir with surgery in 19 AO-RRP patients induced an 89% complete remission rate, with stable disease to a mean of 24 months. See Coulombeau B, Nusa Naiman A, Ceruse P, Froehlich P. Anti-viral injectable treatment (cidofovir) in laryngeal papillomatosis. Rev Laryngol Otol Rhinol (Bord) 2002; 123(5):315-320; Naiman A N, Ayari S, Nicollas R, Landry G, Colombeau B, Froehlich P. Intermediate-term and long-term results after treatment by cidofovir and excision in juvenile laryngeal papillomatosis. Ann Otol Rhinol Laryngol 2006; 115(9):667-672; Naiman A N, Abedipour D, Ayari S, et al. Natural history of adult-onset laryngeal papillomatosis following multiple cidofovir injections. Ann Otol Rhinol Laryngol 2006; 115(3):175-181. Broekema and Dikkers reported that 5 of 188 (2.7%) patients receiving intralesional cidofovir developed dysplasia. However, cidofovir may not be the cause of dysplasia in this cohort since the incidence of spontaneous malignant degeneration in RRP is 2-3%.76 According to a 2013 RRP task force survey of 74 laryngeal surgeons that have used cidofovir to treat RRP, cidofovir may be initiated when surgical debulking is required every two to three months, and dosing should remain below established safe levels (3 mg/kg) and volume. Derkay C S, Volsky P G, Rosen C A, et al. Current use of intralesional cidofovir for recurrent respiratory papillomatosis. Laryngoscope 2013; 123(3):705-712.

### c. Bevacizumab

Bevacizumab is a recombinant monoclonal humanized antibody that blocks angiogenesis by inhibiting the human vascular endothelial growth factor A (VEGF-A). Carifi M, Napolitano D, Morandi M, Dall'Olio D. Recurrent respiratory papillomatosis: current and future perspectives. Ther Clin Risk Manag 2015; 11:731-738. Approved by the FDA in 2004, it was the first angiogenesis inhibitor available in the U.S. and was used as an adjuvant to chemotherapy in metastatic cancers. Shih T, Lindley C. Bevacizumab: An angiogenesis inhibitor for the treatment of solid malignancies. Clin Ther 2006; 28(11):1779-1802. Rahbar et al. conducted a retrospective study to determine the role of VEGF-A in the pathogenesis of RRP patients. Rahbar R, Vargas S O, Folkman J, et al. Role of vascular endothelial growth factor-A in recurrent respiratory papillomatosis. Ann Otol Rhinol Laryngol 2005; 114(4):289-295. Strong expression of VEGF-A mRNA was noted in the squamous epithelium of all 12 RRP patients, and strong expression of VEGFR-1 and VEGFR-2 were noted in the endothelial cells of the papillomas' blood vessels. These observations provided the rationale for assessing the use of bevacizumab in the context of RRP.

Several studies have shown that bevacizumab is relatively safe and active in JO-RRP and AO-RRP. See Ivancic R, Iqbal H, deSilva B, Pan Q, Matrka L. Current and future management of recurrent respiratory papillomatosis. Laryngoscope Investig Otolaryngol. 2018 Jan. 14; 3(1):22-34, Table 3. Sidell et al. treated eight JO-RRP patients by debulking the papillomas with a KTP laser, then administering intralesional bevacizumab at 14.25 mg doses at four- to six-week intervals. Sidell D R, Nassar M, Cotton R T, Zeitels S M, de Alarcon A. High-dose sublesional bevacizumab (avastin) for pediatric recurrent respiratory papillomatosis. Ann Otol Rhinol Laryngol 2014; 123(3):214-221. The median Derkay scores decreased by 58% post-treatment and, moreover, the time between procedures more than doubled. Zeitels et al. studied the efficacy of adjunct bevacizumab with KTP laser excision on 20 adult patients with bilateral vocal fold RRP. Zeitels S M, Barbu A M, Landau-Zemer T, et al. Local injection of bevacizumab (Avastin) and angiolytic KTP laser treatment of recurrent respiratory papillomatosis of the vocal folds: a prospective study. Ann Otol Rhinol Laryngol. 2011; 120(10):627-634. They reported that 19/20 (95%) patients had better disease control in the bevacizumab/KTP laser-treated vocal fold than on the KTP laser-only-treated vocal fold, despite selecting the vocal fold with more extensive disease to receive the bevacizumab/KTP laser treatment. Consistent with these studies, other groups have reported promising results using the combination of KTP laser and bevacizumab, with minimal complications. Sidell D R, Nassar M, Cotton R T, Zeitels S M, de Alarcon A. High-dose sublesional bevacizumab (avastin) for pediatric recurrent respiratory papillomatosis. Ann Otol Rhinol Laryngol 2014; 123(3):214-221; Maturo S, Hartnick C J. Use of 532-nm pulsed potassium titanyl phosphate laser and adjuvant intralesional bevacizumab for aggressive respiratory papillomatosis in children: initial experience. Arch Otolaryngol Head Neck Surg 2010; 136(6):561-565; Best S R, Friedman A D, Landau-Zemer T, et al. Safety and dosing of bevacizumab (avastin) for the treatment of recurrent respiratory papillomatosis. Ann Otol Rhinol Laryngol 2012; 121(9):587-593.; Rogers D J, Ojha S, Maurer R, Hartnick C J. Use of adjuvant intralesional bevacizumab for aggressive respiratory papillomatosis in children. JAMA Otolaryngol Head Neck Surg 2013; 139(5):496-501.

### d. Celecoxib

Celecoxib is a COX-2-selective non-steroidal anti-inflammatory drug used to manage pain and inflammation associated with osteoarthritis, rheumatoid arthritis, ankylosing spondylitis, painful menstruation, and other acute and chronic pain indications. Carifi M, Napolitano D, Morandi M, Dall'Olio D. Recurrent respiratory papillomatosis: current and future perspectives. Ther Clin Risk Manag 2015; 11:731-738. Overexpression of COX-2 was observed in the papillomas of RRP patients, and this increase was proposed to be a consequence of epithelial growth factor receptor (EGFR) and phosphatidylinositol 3-kinase (PI-3K) signaling. Wu R, Abramson A L, Shikowitz M J, Dannenberg A J, Steinberg B M. Epidermal growth factor-induced cyclooxygenase-2 expression is mediated through phosphatidylinositol-3 kinase, not mitogen-activated protein/extracellular signal-regulated kinase, in recurrent respiratory papillomas. Clin Cancer Res 2005; 11(17):6155-6161. In 2009, Limsukon et al. showed success in treating an RRP patient with a combination of celecoxib and erlotinib (a tyrosine kinase inhibitor) at doses of 400 mg per day and 150 mg per day, respectively. Limsukon A, Susanto I, Hoo G W, Dubinett S M, Batra R K. Regression of recurrent respiratory papillomatosis with celecoxib and erlotinib combination therapy. Chest 2009; 136(3):924-926. This patient had several surgical procedures and received IV cidofovir, but her recurrence rate accelerated and her disease began to involve the mainstem and segmental bronchi. The patient underwent a 3-month surveillance bronchoscopy following erlotinib/celecoxib therapy and surprisingly, there was no evidence of disease recurrence.

A randomized double-blind controlled study to determine the safety and efficacy of celecoxib in both pediatric and adult RRP patients was also conducted (NCT 00571701). The primary objective of this trial was to determine the efficacy of celecoxib response in comparison to conventional endoscopy and surgical treatment. All the patients in the study were evaluated under general anesthesia for disease severity at three-month intervals for 30 months; any papillomas present at the time of evaluation were surgically excised. Patients were randomly divided into early and delayed treatment arms. Patients in the early treatment arm received 12 months of 400 mg (adults), 200 mg (pediatric weight greater than 25 kg), or 100 mg (pediatric weight between 12 and 25 kg) of celecoxib daily, then 12 months of placebo daily. The late treatment arm received daily placebo for the first 12 months, then daily celecoxib for the second 12 months. Primary endpoint data showed that celecoxib treatment did not affect the mean percent change in papilloma growth rate at the 12-month measurement compared to baseline (p=0.57). Analysis of secondary outcomes showed no reduction in papilloma growth rate when comparing age (juvenile- vs. adult-onset, p=1.00), gender (male vs. female, p >0.3), or HPV subtype (HPV6 vs. HPV11, p >0.5).

### e. PD-I Inhibitors

Programmed cell death protein 1 (PD-1), which is present on the surface of leukocytes, negatively regulates the immune system when it binds to ligands PD-L1 and PD-L2 on antigen-presenting cells (APCs); PD-L1 has been shown to be highly expressed in HPV-associated head and neck squamous cell carcinoma (HNSCC). Freeman G J, Long A J, Iwai Y, et al. Engagement of the PD-1 immunoinhibitory receptor by a novel B7 family member leads to negative regulation of lymphocyte activation. J Exp Med 2000; 192(7):1027-1034; Lyford-Pike S, Peng S, Young G D, et al. Evidence for a role of the PD-1:PD-L1 pathway in immune resistance of HPV-associated head and neck squamous cell carcinoma. Cancer Res 2013; 73(6):1733-1741. PD-1 inhibitors, such as Pembrolizumab (KEYTRUDA®), Nivolumab (OPDIVO®), and Cemiplimab (LIBTAYO®), block the interaction between PD-1 and its ligands and have clinical efficacy in numerous advanced solid tumors, including HPV-associated HNSCC. Brahmer J R, Drake C G, Wollner I, et al. “Phase I study of single-agent anti-programmed death-1 (MDX-1106) in refractory solid tumors: safety, clinical activity, pharmacodynamics, and immunologic correlates.” J Clin Oncol 2010; 28(19):3167-3175. The activity against HPV-associated HNSCC has prompted investigators to initiate a Phase II clinical trial to assess the efficacy of the PD-1 inhibitor Pembrolizumab in RRP (NCT02632344). RRP patients are being administered 200 mg pembrolizumab as a 30-minute IV infusion every three weeks on day 1 of each cycle, after all procedures and assessments have been completed. This is consistent with current dosing standards in treatment of recurrent or metastatic HNSCC.

### f. EGFR Inhibitors

EGRF inhibitors may be useful in treating or managing RRP. In one study, Gefitinib, an EGFR tyrosine kinase inhibitor, was used in a life-threatening RRP case when all other treatments were exhausted. See Bostrom B, Sidman J, Marker S, Lander T, Drehner D. Gefitinib therapy for life-threatening laryngeal papillomatosis. Arch Otolaryngol Head Neck Surg 2005; 131(1):64-67. This particular case was a 14-year-old black male born with fetal alcohol syndrome. He underwent a tracheostomy at three-months-old due to extensive HPV11-associated RRP and subsequently, disease recurred with complete airway stenosis that extended to the trachea and mainstem bronchi. He was treated with IFN-alpha-2a until he developed hypertension, nephrotic syndrome, and renal failure at age eight. Attempts to control disease with surgical resection, local debulking, oral and inhaled ribavirin (antiviral), indole-3-carbinol, and PDT all failed, and he was not a candidate for cidofovir due to renal failure. After being treated surgically for a life-threatening airway obstruction, gefitinib was administered at a dosage of 250 mg twice daily for 11 months because his papillomas overexpressed EGFR and were becoming increasingly life-threatening. Debulking procedures were significantly reduced from 15 procedures per three months before gefitinib treatment to five procedures per three months during gefitinib treatment, with acceptable toxicity. This study suggests that EGFR inhibitors may be offered as second-line therapy in EGFR-positive RRP.

### g. Other Adjuvants Useful to Treat RRP

Other novel adjuvant approaches have been attempted for the management of RRP, with some clinical success, including those listed out in Table 5.

TABLE 5
Other Adjuvant Therapies for Treatment of RRP.

| | Treatment Type/ | Patients/ | | |
|---|---|---|---|---|
| Treatment | Admin. | Follow-up | Results | Conclusion |
| Acyclovir^{a} | Antiviral agent/ | 3 adults/ | Complete remission | Oral acyclovir as an |
| Antiviral drug that | PO | 1 year | with no residual | adjuvant to surgery |
| targets thymidine kinase | | | disease after 1 year | may reduce |
| expressed by herpes | | | follow-up in 2 | recurrence in RRP; |
| simplex virus-1 and | | | patients | larger cohort studies |
| Epstein-Barr virus, | | | | are needed to assess |
| which are occasional | | | | efficacy |
| concurrent and co- | | | | |
| infections of HPV in | | | | |
| RRP | | | | |
| Ribavirin^{b} | Antiviral agent/ | 4 (1 child; | 2 adults achieved | Ribavirin may be an |
| Antiviral drug that is | PO | 3 adults)/ | minimal recurrence; | effective adjuvant to |
| used to treat respiratory | | 4 months | the other adult and | laser surgery, but |
| syncytial virus | | | child achieved | needs a larger |
| pneumonia in infants | | | increased intervals | controlled clinical |
| and has shown some | | | between surgeries | trial to assess efficacy |
| promise in treating | | | | |
| aggressive RRP | | | | |
| Indole-3-carbinol | Dietary | 33 (9 | After 8 months or | There is potential for |
| (I3C)^{c} | supplement | children; | more of treatment, 11 | I3C as an adjuvant to |
| RRP lesions exhibit | (cruciferous | 24 adults)/ | (33%) patients had | surgery, but larger |
| increased estrogen | vegetables)/ | Mean 4.8 | cessation of | blinded, controlled |
| binding, and a study in | PO | years | papilloma growth | studies need to be |
| mice showed that | | | and did not require | performed |
| inhibition of estrogen | | | further surgery, 10 | |
| metabolism with I3C | | | (30%) had reduced | |
| reduced HPV-induced | | | papilloma growth | |
| papilloma tumor | | | rate, and 12 (36%) | |
| formation by 75%^{d} | | | had no evident | |
| | | | response | |
| Cis-retinoic acid^{e} | Retinoid/ | 9/ | 4/6 (67%) treated | Cis-retinoic acid |
| In the aerodigestive | PO | 18-34 | patients experienced | appears ineffective as |
| tract, vitamin A | | months | recurrence, and all | an adjuvant to surgery |
| deficiency has shown | | | experienced toxicity | in RRP and further |
| increased | | | | studies do not seem |
| hyperkeratinization and | | | | warranted |
| squamous metaplasia, | | | | |
| while excess has shown | | | | |
| to suppress squamous | | | | |
| differentiation and | | | | |
| cause mucous | | | | |
| metaplasia | | | | |

HPV = human papilloma virus;
RRP = Recurrent respiratory papillomatosis
^{a}Chaturvedi J, Sreenivas V, Hemanth V, Nandakumar R. Management of adult recurrent respiratory papillomatosis with oral acyclovir following micro laryngeal surgery: a case series. Indian J Otolaryngol Head Neck Surg 2014; 66(Suppl 1): 359-363.
^{b}McGlennen R C, Adams G L, Lewis C M, Faras A J, Ostrow R S. Pilot trial of ribavirin for the treatment of laryngeal papillomatosis. Head Neck 1993; 15(6): 504-512.
^{c}Rosen C A, Bryson P C. Indole-3-carbinol for recurrent respiratory papillomatosis: long-term results. J Voice 2004; 18(2): 248-253.
^{d}Newfield L, Goldsmith A, Bradlow H L, Auborn K. Estrogen metabolism and human papillomavirus-induced tumors of the larynx: chemo-prophylaxis with indole-3-carbinol. Anticancer Res 1993; 13(2): 337-341.
^{e}Bell R, Hong W K, Itri L M, McDonald G, Strong M S. The use of cis-retinoic acid in recurrent respiratory papillomatosis of the larynx: a randomized pilot study. Am J Otolaryngol 1988; 9(4): 161-164.

### D. Other Combination Therapies

It is also contemplated that other agents may be used in combination with certain aspects of the present embodiments to improve the therapeutic efficacy of the treatment. These additional agents include agents that affect up-regulation of cell surface receptors and GAP junctions, cell proliferation inhibitors and differentiators, cell adhesion inhibitors, agents that increase the sensitivity of hyperproliferative cells to apoptosis inducers, or other biological agents. An increase in intercellular signaling due to an increase in the number of GAP junctions will enhance the anti-hyperproliferative effect on adjacent hyperproliferative cell populations.

In other embodiments, cell proliferation inhibition or differentiation agents can be used in combination with certain aspects of this embodiment to improve the anti-hyperproliferative efficacy of the treatment. It is believed that cell adhesion inhibitors can improve the efficacy of this embodiment. Examples of cell adhesion inhibitors include topical adhesion kinase (FAK) inhibitors and lovastatin. Additionally, it is contemplated that other agents, such as antibody c225, that increase the sensitivity of the hyperproliferative cells to apoptosis, may be used in combination with certain aspects of this embodiment to improve therapeutic efficacy.

### VIII. Pharmaceutical Compositions

The therapeutic compounds, including viral vectors, ligands, and corticosteroids, as described herein, can be integrated into pharmaceutical compositions suitable for administration. These compositions typically consist of the therapeutic compound(s) and a pharmaceutically acceptable carrier.

In certain embodiments, the pharmaceutical composition includes the vector described herein and a carrier, with specific mention of adenovirus or adenoviral vector in some embodiments.

Proper formulation of the pharmaceutical composition is dependent upon the route of administration chosen. A summary of pharmaceutical compositions described herein is found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A, and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Any suitable carrier can be used within the context of the present disclosure, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable pharmaceutical compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The pharmaceutical composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, (e.g., intravenous, intradermal, subcutaneous) oral (including, inhalation), topical; (i.e., transdermal), transmucosal, or rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the therapeutic compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the therapeutic compound(s) are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The therapeutic compound(s) can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of therapeutic compound(s) calculated to produce the desired therapeutic effect in association with the required pharmaceutical carrier. The specification for the dosage unit forms of the disclosure are dictated by and directly dependent on the unique characteristics of the therapeutic compound(s) and the particular therapeutic effect to be achieved, and the limitations inherent in the art of compounding such a therapeutic compound(s) for the treatment of individuals.

The pharmaceutical compositions can be included in a container, pack, or dispenser together with instructions for administration.

In some embodiments, the pharmaceutical composition comprising, e.g., the vector is formulated to protect the vector from damage prior to administration. For example, the pharmaceutical composition can be formulated to reduce loss of the vector on devices used to prepare, store, or administer the vector, such as glassware, syringes, or needles. The pharmaceutical composition can be formulated to decrease the light sensitivity and/or temperature sensitivity of the vector. To this end, the pharmaceutical composition preferably comprises a pharmaceutically-acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition will extend the shelf life of the adenovirus or adenoviral vector, and facilitate its administration. Formulations for vector-containing compositions are further described in, for example, U.S. Pat. Nos. 6,225,289, 6,514,943, and International Patent Application Publication WO 2000/034444.

The pharmaceutical composition also can be formulated to enhance transduction efficiency. In addition, the skilled artisan will appreciate that the polynucleotide, polypeptide, vector, vaccine, or cell can be present in a composition with other therapeutic or biologically-active agents. For example, factors that control inflammation, such as ibuprofen or steroids, can be part of the composition to reduce swelling and inflammation associated with in vivo administration of the polynucleotide, polypeptide, vector, composition, vaccine, or cell. For example, in cases where an adenovirus or adenoviral vector is used to deliver an antigen-encoding nucleic acid sequence to a host, immune system stimulators or adjuvants, e.g., interleukins, lipopolysaccharide, or double-stranded RNA, may be administered to enhance or modify any immune response to the antigen. Antibiotics (e.g., microbicides and fungicides) can be present to treat existing infection and/or reduce the risk of future infection, such as infection associated with gene transfer procedures.

In certain embodiments, the pharmaceutical compositions may include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the pharmaceutical composition in an acceptable range.

In certain embodiments, the pharmaceutical composition may comprise one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical composition may be formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

In certain embodiments, the pharmaceutical composition is a liquid. In some embodiments, the composition may be lyophilized and then reconstituted before use.

In certain embodiments, pharmaceutical compositions may include one or more preservatives, for example, to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, the pharmaceutical composition may include one or more antifoaming agents. Antifoaming agents can reduce foaming during processing which can result in coagulation of aqueous dispersions, bubbles in the finished film, or generally impair processing. Exemplary anti-foaming agents include silicon emulsions or sorbitan sesquoleate.

In certain embodiments, the pharmaceutical composition may include one or more antioxidants. Exemplary antioxidants include butylated hydroxytoluene (BHT), sodium ascorbate, ascorbic acid, sodium metabisulfite and tocopherol. In certain embodiments, the one or more antioxidants enhance chemical stability of the composition.

In certain embodiments, the pharmaceutical composition may include one or more stabilizing agents. Exemplary stabilizing agents include, for example: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In certain embodiments, the pharmaceutical composition may include one or more binders. Binders can impart cohesive qualities. Exemplary binders include: alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

In certain embodiments, the pharmaceutical composition may include a carrier or a pharmaceutically-compatible carrier material. These may include any commonly used excipients in pharmaceutics and should be selected on the basis of compatibility with the pharmaceutical compounds described herein. Exemplary carrier materials include binders, suspending agents, disintegration agents, filling agents, surfactants, solubilizers, stabilizers, lubricants, wetting agents, diluents, and the like. Exemplary pharmaceutically-compatible carrier materials may include acacia, gelatin, colloidal silicon dioxide, calcium glycerophosphate, calcium lactate, maltodextrin, glycerin, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A, and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition may include one or more diffusion facilitating agents, dispersing agents, and/or viscosity modulating agents, for example, to control the diffusion and homogeneity of the composition through liquid media or a granulation or blend method. In some embodiments, these agents also facilitate the effectiveness of a coating or eroding matrix. Exemplary diffusion facilitators and dispersing agents may include hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

In certain embodiments the pharmaceutical composition comprises a combination of one or more erosion facilitators with one or more diffusion facilitators.

In certain embodiments, the pharmaceutical composition may include one or more diluents. A diluent is a chemical compound that is used to dilute the substance of interest prior to delivery. Diluents can also be used to stabilize substances because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the pharmaceutical composition may include one or more filling agent. Filling agents may include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the pharmaceutical composition may include one or more lubricants or glidants. These are compounds that prevent, reduce, or inhibit adhesion or friction of materials. Exemplary lubricants may include compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

In certain embodiments, the pharmaceutical composition may include one or more plasticizers. These are compounds used to soften the microencapsulation material or film coatings to make them less brittle. Exemplary plasticizers may include polyethylene glycols such as PEG 300, PEG 400, PEG 600, PEG 1450, PEG 3350, and PEG 800, stearic acid, propylene glycol, oleic acid, triethyl cellulose and triacetin. In some embodiments, the plasticizers may also function as dispersing agents or wetting agents.

In certain embodiments, the pharmaceutical composition may include one or more solubilizers. Exemplary solubilizers may include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In certain embodiments, the pharmaceutical composition may include one or more stabilizers. Exemplary stabilizers may include any antioxidation agents, buffers, acids, preservatives and the like.

In certain embodiments, the pharmaceutical composition may include one or more suspending agents. Exemplary suspending agents may include compounds such as polyvinylpyrrolidone (e.g., polyvinylpyrrolidone K12, polyvinylpyrrolidone K17, polyvinylpyrrolidone K25, or polyvinylpyrrolidone K30), vinyl pyrrolidone/vinyl acetate copolymer (S630), polyethylene glycol (e.g., the polyethylene glycol can have a molecular weight of about 300 to about 6000, or about 3350 to about 4000, or about 7000 to about 5400), sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics (e.g., sodium carboxymethylcellulose, methylcellulose, sodium carboxymethylcellulose, hydroxypropylmethylcellulose, hydroxyethylcellulose), polysorbate-80, sodium alginate, polyethoxylated sorbitan monolaurate, polyethoxylated sorbitan monolaurate, povidone and the like.

In certain embodiments, the pharmaceutical composition may include one or more surfactants. Exemplary surfactants may include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, the surfactants can be included in the pharmaceutical composition to enhance physical stability or for other purposes.

In certain embodiments, the pharmaceutical composition may include one or more viscosity enhancing agents. Exemplary viscosity enhancing agents may include methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

In certain embodiments, the pharmaceutical composition may include one or more wetting agents. Exemplary wetting agents may include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In certain embodiments, the pharmaceutical composition may be manufactured in a conventional manner, such as by means of conventional mixing, dissolving, granulating, dragee-making, levigating, emulsifying, encapsulating, entrapping, or compression processes.

In certain embodiments, the pharmaceutical composition for administration of the vector (e.g., an adenoviral vector) described herein, may conveniently be presented in unit dosage form and be prepared by any of the methods well known in the art of pharmacy. In general, the pharmaceutical compositions may be prepared by bringing the active ingredient into association with a carrier, and then, if necessary, shaping the product into the desired formulation. In the pharmaceutical composition, the polynucleotide, polypeptide, vector, vaccine, or cell described herein is included in an amount sufficient to produce the desired effect upon the process, condition or disease sought to be treated.

In certain embodiments, the pharmaceutical composition comprises the polynucleotide, polypeptide, vector, vaccine, or cell described herein in a therapeutically-effective amount. An “effective amount” is any amount required to demonstrate a therapeutic effect in the subject. The amount may vary depending on the subject's condition, age, gender, medical history, and/or weight. The dosage may also vary depending on the condition to be treated, the anti-inflammatory agent encoded, the type of polynucleotide, polypeptide, vector, vaccine, or cell being administered, and/or the route of administration.

In some embodiments, the pharmaceutical composition comprises the vector described herein at a concentration of about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

In some embodiments, the pharmaceutical composition may be stored by freezing at a temperature of about 0° C. to about −120° C., about −10° C. to about −110° C., about −20° C. to about −100° C., about −30° C. to about −90° C., about −40° C. to about −90° C., about −50° C. to about −90° C., about −60° C. to about −90° C., about −65° C. to about −85° C., or about −70° C. to about −80° C. In some embodiments, the pharmaceutical composition may be stored by freezing at a temperature of about −60° C., about −61° C., about −62° C., about −63° C., about −64° C., about −65° C., about −66° C., about −66° C., about −67° C., about −68° C., about −69° C., about −70° C., about −71° C., about −72° C., about −73° C., about −74° C., about −75° C., about −76° C., about −77° C., about −78° C., about −79° C., about −80° C., about −81° C., about −82° C., about −83° C., about −84° C., about −85° C., about −86° C., about −87° C., about −88° C., about −89° C., or about −90° C. The pharmaceutical composition may be thawed, for example, in a water bath prior to use, avoiding prolonged exposure of the thawed composition to the water bath. The temperature of water bath used to thaw the pharmaceutical composition may be, for example, between about 30° C. to about 44° C., about 31° C. to about 43° C., about 32° C. to about 42° C., about 33° C. to about 41° C., about 34° C. to about 40° C., or about 35° C. to about 39° C. In some embodiments, the temperature of water bath used to thaw the pharmaceutical composition may be about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. The thawed composition may be stored for up to about 15 minutes, up to about 30 minutes, up to about 45 minutes, up to about 1 hour, up to about 75 minutes, up to about 90 minutes, up to about 105 minutes, or up to about 2 hours at ambient temperature prior to administration. In some embodiments, the thawed composition will appear as a clear to slightly opalescent, colorless liquid and be substantially free of visible particulates.

### IX. Kits/Articles of Manufacture

Another aspect of the present invention is are kits and articles of manufacture for use with one or more methods described herein. Suitable kits may include a package or container that comprise the polynucleotide, polypeptide, vector, vaccine, or cell described herein, or the composition comprising the same. Suitable containers include, for example, bottles, vials, syringes, and test tubes. In some embodiments, the containers are formed from a variety of materials, such as glass or plastic. Suitable articles of manufacture may contain packaging materials. Examples of pharmaceutical packaging materials include blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions may also be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

In some embodiments, the kit contains a vial containing the composition of the present invention. In some such embodiments, the vial comprises, for example, from about 0.1 to about 20 ml of the pharmaceutical composition, from about 0.1 to about 15 ml of the pharmaceutical composition, from about 0.1 to about 10 ml of the pharmaceutical composition, from about 0.1 to about 9 ml of the pharmaceutical composition, from about 0.1 to about 8 ml of the pharmaceutical composition, from about 0.1 to about 7 ml of the pharmaceutical composition, from about 0.1 to about 6 ml of the pharmaceutical composition, from about 0.1 to about 5 ml of the pharmaceutical composition, from about 0.2 to about 5 ml of the pharmaceutical composition, from about 0.2 to about 4 ml of the pharmaceutical composition, from about 0.2 to about 3 ml of the pharmaceutical composition, from about 0.2 to about 2 ml of the pharmaceutical composition, from about 0.2 to about 1 ml of the pharmaceutical composition, from about 0.5 to about 2 ml of the pharmaceutical composition, from about 0.5 to about 1.75 ml of the pharmaceutical composition, from about 0.5 to about 1.5 ml of the pharmaceutical composition, from about 0.5 to about 1.25 ml of the pharmaceutical composition, from about 0.5 to about 1 ml of the pharmaceutical composition, from about 0.75 to about 1.25 ml of the pharmaceutical composition. In other such embodiments, the vial comprises, for example, about 0.5 ml of the pharmaceutical composition, about 0.55 ml of the pharmaceutical composition, about 0.6 ml of the pharmaceutical composition, about 0.65 ml of the pharmaceutical composition, about 0.7 ml of the pharmaceutical composition, about 0.75 ml of the pharmaceutical composition, about 0.8 ml of the pharmaceutical composition, about 0.85 ml of the pharmaceutical composition, about 0.9 ml of the pharmaceutical composition, about 0.95 ml of the pharmaceutical composition, about 1 ml of the pharmaceutical composition, about 1.05 ml of the pharmaceutical composition, about 1.1 ml of the pharmaceutical composition, about 1.15 ml of the pharmaceutical composition, or about 1.2 ml of the pharmaceutical composition.

In some embodiments, the viral vector contained within the composition may be present at a concentration of, for example, about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

### X. Methods of Evaluating Efficacy of Treatment

Regardless of the method used to introduce exogenous nucleic acids into a host cell or otherwise expose a cell to the inhibitor of the present disclosure, in order to confirm the presence of the recombinant DNA sequence in the host cell, a variety of assays can be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

### A. Immune Phenotyping

Exploratory immunologic studies may also be conducted to evaluate the study drug's effect on the immune response before and after treatment, to gain insight into potential biomarkers, and help improve the administered therapy. In one embodiments, for example, such immunologic assays may be performed at the Laboratory of Tumor Immunology and Biology (LTIB) at the NCI's Center for Cancer Research (CCR) in select participants where adequate samples are available. In certain embodiments, the samples collected from participants are blood samples. In other embodiments, the samples may be saliva samples. The immunologic assays may include: (i) analyzing PBMCs for changes in standard immune cell types (CD4 and CD8 T cells, natural killer (NK) cells, regulatory T cells (Tregs), myeloid-derived suppressor cells (MDSCs), and dendritic cells) as well as 123 immune cell subsets, using multi-color flow cytometry; (ii) analyzing PBMCs from selected subjects for function of specific immune cell subsets, including CD4 and CD8 T cells, NK cells, Tregs, and MDSCs using flow-based assays; and/or (iii) analyzing PBMCs for tumor antigen-specific immune responses using an intracellular cytokine staining assay. PBMCs will be stimulated in vitro with overlapping 15-mer peptide pools encoding the tumor-associated HPV6/11 antigens; control peptide pools will involve the use of human leukocyte antigen peptide as a negative control and CEFT peptide mix as a positive control. CEFT is a mixture of peptides of CMV, Epstein-Barr virus, influenza, and tetanus toxin. Post-stimulation analyses of CD4 and CD8 T cells may involve the production of IFN-γ, IL-2, TNF, and the degranulation marker CD107a. If sufficient PBMCs are available, assays may also be performed for the development of T cells to other tumor-associated antigens.

### 1. Soluble Factors

In certain embodiments, Sera may be analyzed pre- and post-therapy for the following soluble factors: sCD27, sCD40 ligand using commercial ELISA kits. For example, in one embodiment, Sera may be analyzed for changes in cytokines (IFN-γ, IL-10, IL-12, IL-2, IL-4, etc.), chemokines, antibodies, tumor-associated antigens, and/or other markers using ELISA or multiplexed assays (e.g., Mesoscale, Luminex, cytokine bead array).

### 2. Anti-Vector Antibodies (AVA)

Anti-vector antibodies are one mechanism of neutralization and inefficacy of viral vectors. Longitudinal detection and titer measurement of AVA to the vaccine adenoviral vector may assist in characterizing cases of loss of efficacy.

### 3. Pharmacokinetics (“PK”)

PK measurements of drugs used in combination with the vaccine adenoviral vector of the present invention disclosed herein may be taken to collect data which will provide insight into population PKs of such drug(s) in participants receiving these novel combinations. For example, in a certain embodiment where the vaccine adenoviral vector is administered alongside a chemotherapeutic agent, PK measurements of the chemotherapy agent may be taken to collect data which may provide insight into population PKs of the chemotherapy agent in participants receiving such novel combination.

### B. Anti-Drug Antibody (“ADA”)

Anti-Drug Antibody development is an accepted mechanism of loss of efficacy of administered human monoclonal antibodies. In certain embodiments of the present invention, measuring titers will ensure that lack of efficacy of a certain combination drug is not due to ADA development. For example, in a certain embodiment where the vaccine adenoviral vector is administered alongside a chemotherapy agent, measuring titers may ensure that lack of efficacy of the chemotherapy agent is not due to ADA development.

### C. Tumor Tissue Analyses for Immune Markers

In other embodiments, analyses may be performed in tumor tissue pre-treatment vs. post-treatment with the vaccine adenoviral vector. In certain embodiments, archival tumor samples may be requested for pre-treatment analysis. In another embodiment, preferably tissue samples from the last 6 months may be analyzed. In yet another embodiment, for participants with lesions amenable to biopsy, two biopsies may be performed at baseline, and at Week 4-5 or Week 9 post-treatment. In certain embodiments, the study of immune infiltration as well as PD-L1 status within the tumor microenvironment pre vs. post treatment by immunohistochemistry and/or multiplex immunofluorescence may be performed.

### D. Single-Cell Proteomic Analysis

In another embodiment, tumor tissue single-cell proteomic analysis of immune and signaling pathways may be performed with the Isoplexis Single Cell Functional proteomic platform by Precigen.

### E. Immune Transcriptomic Analysis

In one embodiment, tumor tissue immune transcriptomic analysis may be performed with the Nanostring platform by Precigen.

### F. Assessment of HPV Status

In yet another embodiment, where participants have available tumor tissue (either archival or by optional biopsy), HPV testing may be performed using the Roche Cobas or Becton Dickinson HPV PCR based DNA assay, if no prior HPV testing of the tumor has been performed.

### G. Circulating Tumor DNA

In another embodiment, where sufficient plasma is available, select participant samples may be analyzed for circulating tumor DNA. Plasma DNA may be isolated with an automated purification system, and the circulating tumor/HPV DNA may be quantified with a digital droplet PCR system from Bio-Rad to obtain precise quantification.

In a further embodiment, RNA expression and T-cell receptor clonality analysis may be done on the peripheral blood, as well as archived tumor tissue or optional biopsies, to help further evaluate changes in immune response and RNA expression levels with treatment, as well as to determine tumor and infiltrating lymphocyte characteristics which may be predictive of response to treatment. In addition, such analyses may also be used to gauge resistance mechanisms and additional targets for future therapy. Coded, linked samples, may be analyzed for RNA expression levels using the Nanostring platform and T-cell receptor clonality using the ImmunoSeq platform.

### H. Salivary HPV DNA

In a certain embodiment, saliva samples may be analyzed for HPV DNA detection and quantification. Salivary HPV DNA may be quantified with digital droplet PCR with no sequencing is involved. In one embodiment, participants with p16-positive oropharyngeal cancer may provide a saliva sample by mouth rinse and gargle with 15-20 mL 0.9% NaCl for 30 seconds and spitting into the collection tube.

### Exemplary Embodiments of the Invention

The foregoing description of specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. Some additional exemplary embodiments (“E”) of the invention include, but are not limited to:

E1. A polynucleotide encoding a non-naturally occurring polypeptide comprising at least one epitope derived from a low cancer risk human papilloma virus (HPV) protein and at least one epitope derived from a high cancer risk HPV protein.

E2. A polynucleotide encoding a non-naturally occurring polypeptide comprising at least one epitope derived from human papilloma virus 6 (HPV6) and at least one epitope derived from human papilloma virus 11 (HPV11).

E3. The polynucleotide of E2, wherein the non-naturally occurring polypeptide further comprises at least one epitope derived from human papilloma virus 16 (HPV16).

E4. A polynucleotide encoding a non-naturally occurring polypeptide comprising HPV epitopes derived from any four of:
an HPV6 E2 protein;
an HPV6 E4 protein;
an HPV6 E6 protein;
an HPV6 E7 protein;
an HPV11 E6; and/or
an HPV11 E7 protein;
wherein the polypeptide comprises at least one HPV6 epitope and at least one HPV11 epitope.

E5. The polynucleotide of E4, wherein the non-naturally occurring polypeptide further comprises at least one epitope derived from HPV16 proteins.

E6. A polynucleotide encoding a non-naturally occurring polypeptide comprising HPV epitopes derived from any three of:
an HPV6 E2 protein;
an HPV6 E4 protein;
an HPV6 E6 protein; or
an HPV6 E7 protein;
wherein the HPV epitopes are linked by amino acid sequences not naturally encoded by an HPV genome.

E7. A polynucleotide encoding a non-naturally occurring polypeptide comprising HPV epitopes derived from each of:
an HPV6 E2 protein;
an HPV6 E4 protein;
an HPV6 E6 protein; and
an HPV6 E7 protein;
wherein the HPV epitopes are linked by amino acid sequences not naturally encoded by an HPV genome.

E8. The polynucleotide of E6 or E7, wherein an amino acid sequence linking at least two of the HPV epitopes comprises the sequence of SEQ ID NO: 34.

E9. A polynucleotide encoding a non-naturally occurring polypeptide, wherein the non-naturally occurring polypeptide comprises HPV epitopes derived from:
an HPV6 E2 protein, an HPV6 E4 protein, an HPV6 E6 protein, an HPV6 E7 protein, an HPV11 E6 protein, and an HPV11 E7 protein; or,
an HPV6 E2 protein, an HPV6 E4 protein, an HPV6 E6 protein, an HPV6 E7 protein, an HPV11 E6 protein, an HPV11 E7 protein; an HPV16 E6 protein, and an HPV16 E7 protein.

E10. The polynucleotide of E9, further comprising one or more polynucleotide sequences encoding one or more polypeptide linkers, wherein the one or more linkers comprise an amino acid sequence of:
a) SEQ ID NO: 45;
b) SEQ ID NO: 42;
c) SEQ ID NO: 40;
d) SEQ ID NO: 34;
e) SEQ ID NO: 52;
f) SEQ ID NO: 48;
g) SEQ ID NO: 50; and/or,
h) SEQ ID NO: 57.

E11. A polynucleotide encoding a non-naturally occurring polypeptide, wherein the non-naturally occurring polypeptide comprises HPV epitopes derived from:
a) an HPV6 E2 protein, an HPV6 E4 protein, an HPV6 E6 protein, an HPV6 E7 protein, an HPV11 E6 protein, and an HPV11 E7 protein, wherein the polypeptide further comprises at least one linker sequence of SEQ ID NO: 45, SEQ ID NO: 42, or SEQ ID NO: 40;
b) an HPV6 E2 protein, an HPV6 E4 protein, an HPV6 E6 protein, an HPV6 E7 protein, an HPV11 E6 protein, and an HPV11 E7 protein, wherein the polypeptide further comprises at least one linker sequence of SEQ ID NO: 34;
c) an HPV6 E2 protein, an HPV6 E4 protein, an HPV6 E6 protein, an HPV11 E6 protein, an HPV11 E7 protein, an HPV16 E6 protein; an HPV16 E7 protein, wherein the polypeptide further comprises at least one linker sequence of SEQ ID NO: 52, SEQ ID NO: 48, or SEQ ID NO: 50; or,
d) an HPV6 E2 protein, an HPV6 E4 protein, an HPV6 E6 protein, an HPV11 E6 protein, an HPV11 E7 protein, an HPV16 E6 protein; and an HPV16 E7 protein, wherein the polypeptide further comprises at least one linker sequence of SEQ ID NO: 57.

E12. A polynucleotide comprising a sequence at least 80% identical, at least 85% identical, at least 90% identical, at least 95% identical, at least 98% identical or I 00% identical to:
a) SEQ ID NO: 67;
b) SEQ ID NO: 69;
c) SEQ ID NO: 71;
d) SEQ ID NO: 73; or,
e) SEQ ID NO: 75.

E13. A polynucleotide comprising a sequence encoding a non-naturally occurring polypeptide comprising an amino acid sequence at least 80% identical to:
a) SEQ ID NO: 66;
b) SEQ ID NO: 68;
c) SEQ ID NO: 70;
d) SEQ ID NO: 72; or,
e) SEQ ID NO: 74.

E14. The polynucleotide of E13, wherein the amino acid sequence is at least 85% identical, at least 90% identical, at least 95% identical, at least 98% identical, or is 100% identical to any one of a), b), c), d) or e).

E15. The polynucleotide in any one of E1 to E14, wherein one or more of the HPV epitopes comprise amino acid substitutions, amino acid insertions, amino sequence deletions, amino acid sequence separations or amino acid sequence rearrangements as compared to the corresponding wild type HPV sequences, wherein said amino acid sequence substitutions, insertions, deletions, separations, or rearrangements function to eliminate naturally-occurring HPV oncogenic biologic activity or essential viral replication functions.

E16. The polynucleotide in any one of E1 to E15, wherein said polynucleotide is useful in generating an in vivo immune response to HPV6 or HPV11 antigens in a mammal, or wherein said polynucleotide is useful in generating in vivo immune responses to HPV6 and HPV11 antigens in a mammal.

E17. A polypeptide encoded by the polynucleotide in any one of E1 to E16.

E18. A vector comprising the polynucleotide in any one of E1 to E16.

E19. The vector of E18, wherein said vector is a plasmid, a viral, or a non-viral vector.

E20. The vector of E19, wherein said viral vector is an adenoviral vector.

E21. The vector of E20, wherein said adenoviral vector is a non-human primate viral vector.

E22. A composition or vaccine comprising a pharmaceutically acceptable carrier and the polynucleotide of any one of E1 to E16, the polypeptide of E17, or the vector of E18 to E21.

E23. A method of generating an HPV6 and HPV11 composition, vaccine or polynucleotide encoding a non-naturally occurring polypeptide for use in a composition or vaccine, the method comprising engineering HPV polynucleotide sequences to encode derivatives of naturally occurring HPV polypeptides, wherein at least one of the viral polypeptides has oncogenic biologic activity or an essential viral replication function, wherein a combination of one or more amino acid sequence substitutions, deletions, insertions, separations, or rearrangements are expressed via the composition, vaccine or encoded by the polynucleotide to generate derivative viral polypeptides, wherein HPV oncogenic biologic activity or essential HPV replication function is eliminated from the derivative viral polypeptides, but wherein host immune-responsiveness and/or immunogenic activity against the derivative viral polypeptides is retained or enhanced compared to the corresponding naturally occurring viral polypeptides.

E24. The method of E23, wherein the polynucleotide or derivative viral polypeptide is designed utilizing steps comprising identification of viral antigens combined with any two or more of: in silico prediction of in vivo antigen epitope recognition, in silico prediction of in vivo allergen recognition, human proteome cross-reactivity sequence homology analysis, amino acid sequence physiochemical property analysis, three-dimensional (3D) structure analysis, identification of oncogenic and/or viral function inactivation mutations, deletions, substitutions and/or rearrangements.

E25. The method of E24, further comprising in vitro or in vivo evaluation of immunogenicity, immune-protective effects, oncogenic, or viral function of the derivative viral polypeptides.

Additional embodiments (“E”) of the invention may also be based on the antigen designs disclosed below in Table 6:

TABLE 6
Nomenclature for Antigen Designs and Component Parts

| Assigned | SEQ ID NO | | |
|---|---|---|---|
| Component | & Sequence | | Sequence |
| Symbol | Type | Descriptive name | type |
| A | 1 AA | HPV6 E2* | Amino acid |
| | 2 DNA | HPV6 E2* | Nucleic acid |
| B | 3 AA | HPV6 E4 1-56 | Amino acid |
| | 4 DNA | HPV6 E4 1-56 | Nucleic acid |
| C | 5 AA | HPV6 E7 70-98 | Amino acid |
| | 6 DNA | HPV6 E7 70-98 | Nucleic acid |
| D | 7 AA | HPV6 E4 36-90 | Amino acid |
| | 8 DNA | HPV6 E4 36-90 | Nucleic acid |
| E | 9 AA | HPV6 E7 1-58 | Amino acid |
| | 10 DNA | HPV6 E7 1-58 | Nucleic acid |
| F | 11 AA | HPV6E6* | Amino acid |
| | 12 DNA | HPV6E6* | Nucleic acid |
| G | 13 AA | HPV6 E2* 1-368 Del 206-306 | Amino acid |
| | 14 DNA | HPV6 E2* 1-368 Del 206-306 | Nucleic acid |
| H | 15 AA | HPV6E4 1-70Del 21-50 | Amino acid |
| | 16 DNA | HPV6E4 1-70Del 21-50 | Nucleic acid |
| I | 17 AA | HPV6 E4 51-70 | Amino acid |
| | 18 DNA | HPV6 E4 51-70 | Nucleic acid |
| J | 19 DNA | HPV6 E4 51-70 | Nucleic acid |
| | 20 AA | HPV6 E6* 1-120 Del C-term | Amino acid |
| | 21 DNA | HPV6 E6* 1-120 Del C-term | Nucleic acid |
| K | 22 AA | HPV6 E7 1-98 Del 40-75 | Amino acid |
| | 23 DNA | HPV6 E7 1-98 Del 40-75 | Nucleic acid |
| L | 24 AA | HPV6 E2* 1-205 | Amino acid |
| | 25 DNA | HPV6 E2* 1-205 | Nucleic acid |
| M | 26 AA | HPV6 E4 1-20 | Amino acid |
| | 27 DNA | HPV6 E4 1-20 | Nucleic acid |
| N | 28 AA | HPV6 E2* 307-368 | Amino acid |
| | 29 DNA | HPV6 E2* 307-368 | Nucleic acid |
| O | 30 AA | HPV6 E4 Epitope-No. 1 | Amino acid |
| | 31 DNA | HPV6 E4 Epitope-No. 1 | Nucleic acid |
| P | 32 AA | HPV6 E4 Epitope-No. 2 | Amino acid |
| | 33 DNA | HPV6 E4 Epitope-No. 2 | Nucleic acid |
| L1 | 34 AA | Rigid linker | Amino acid |
| | 35 DNA | Rigid linker | Nucleic acid |
| | 36 DNA | Rigid linker | Nucleic acid |
| | 37 DNA | Rigid linker | Nucleic acid |
| | 38 DNA | Rigid linker | Nucleic acid |
| | 39 DNA | Rigid linker | Nucleic acid |
| L2 | 40 AA | HPV6 E6 epitope linker | Amino acid |
| | 41 DNA | HPV6 E6 epitope linker | Nucleic acid |
| L3 | 42 AA | HPV11 E6 epitope linker | Amino acid |
| | 43 DNA | HPV11 E6 epitope linker | Nucleic acid |
| | 44 DNA | HPV11 E6 epitope linker | Nucleic acid |
| L4 | 45 AA | HPV11 E7 epitope linker | Amino acid |
| | 46 DNA | HPV11 E7 epitope linker | Nucleic acid |
| | 47 DNA | HPV11 E7 epitope linker | Nucleic acid |
| L5 | 48 AA | HPV16 E6 Agonist Enhancer | Amino acid |
| | | No. 1 | |
| | 49 DNA | HPV16 E6 Agonist Enhancer | Nucleic acid |
| | | No. 1 | |
| L6 | 50 AA | HPV16 E6 Agonist Enhancer | Amino acid |
| | | No. 2 | |
| | 51 DNA | HPV16 E6 Agonist Enhancer | Nucleic acid |
| | | No. 2 | |
| L7 | 52 AA | HPV16 E7 agonist enhancer | Amino acid |
| | 53 DNA | HPV16 E7 agonist enhancer | Nucleic acid |
| | 54 DNA | HPV16 E7 agonist enhancer | Nucleic acid |
| | 55 DNA | HPV16 E7 agonist enhancer | Nucleic acid |
| | 56 DNA | HPV16 E7 agonist enhancer | Nucleic acid |
| L8 | 57 AA | Flex linker | Amino acid |
| | 58 DNA | Flex linker | Nucleic acid |
| | 59 DNA | Flex linker | Nucleic acid |
| | 60 DNA | Flex linker | Nucleic acid |
| | 61 DNA | Flex linker | Nucleic acid |
| | 62 DNA | Flex linker | Nucleic acid |
| | 63 DNA | Flex linker | Nucleic acid |
| | 64 DNA | Flex linker | Nucleic acid |
| | 65 DNA | Flex linker | Nucleic acid |
| D1 | 66 AA | Design No. 1 | Amino acid |
| | 67 DNA | Design No. 1 | Nucleic acid |
| D2 | 68 AA | Design No. 2 | Amino acid |
| | 69 DNA | Design No. 2 | Nucleic acid |
| D3 | 70 AA | Design No. 3 | Amino acid |
| | 71 DNA | Design No. 3 | Nucleic acid |
| D4 | 72 AA | Design No. 4 | Amino acid |
| | 73 DNA | Design No. 4 | Nucleic acid |
| D5 | 74 AA | Design No. 5 | Amino acid |
| | 75 DNA | Design No. 5 | Nucleic acid |
| L9 | 76 AA | Rigid linker polypeptide | Amino acid |
| | 77 DNA | Rigid linker DNA | Nucleic acid |
| | 78 DNA | Rigid linker DNA | Nucleic acid |
| | 79 DNA | Rigid linker DNA | Nucleic acid |
| | 80 DNA | Rigid linker DNA | Nucleic acid |
| | 81 DNA | Rigid linker DNA | Nucleic acid |

E26. A non-naturally occurring polypeptide or polynucleotide, wherein the polypeptide or polynucleotide comprises sequences of any two or more components of Table 6.

E27. A non-naturally occurring polypeptide or polynucleotide, wherein the polypeptide or polynucleotide comprises sequences of any two or more of the components A, B, C, D, E, F, G, H, I, J, K, L, M, N, O, or P of Table 6.

E28. A non-naturally occurring polypeptide or polynucleotide, wherein the polypeptide or polynucleotide comprises sequences of any one or more of the components LI, L2, L3, L4, L5, L6, L7, L8 or L9 of Table 6.

E29. A non-naturally occurring polypeptide or polynucleotide, wherein the polypeptide or polynucleotide comprises sequences of any two or more of the components A, B, C, D, E, or F of Table 6.

E30. A non-naturally occurring polypeptide or polynucleotide, wherein the polypeptide or polynucleotide comprises sequences of any two or more of the components A, B, C, D, E, F, L1, L2, L3, or L4 of Table 6.

E31. A non-naturally occurring polypeptide or polynucleotide, wherein the polypeptide or polynucleotide comprises sequences of any two or more of the components A, B, C, D, E, F, or L1 of Table 6.

E32. A non-naturally occurring polypeptide or polynucleotide, wherein the polypeptide or polynucleotide comprises sequences of any two or more of the components A, B, C, D, E, F, L2, L3, or L4 of Table 6.

E33. A non-naturally occurring polypeptide or polynucleotide, wherein the polypeptide or polynucleotide comprises sequences of components A, B, C, D, E, F and L1 of Table 6.

E34. A non-naturally occurring polypeptide or polynucleotide, wherein the polypeptide or polynucleotide comprises sequences of components A, B, C, D, E, F, L2, L3 and L4 of Table 6.

E35. A non-naturally occurring polypeptide or polynucleotide, wherein the polypeptide or polynucleotide comprises sequences of any two or more of the components G, H, I, J, or K of Table 6.

E36. A non-naturally occurring polypeptide or polynucleotide, wherein the polypeptide or polynucleotide comprises sequences of any two or more of the components G, H, I, J, K, L1, L2, or L4 of Table 6.

E37. A non-naturally occurring polypeptide or polynucleotide, wherein the polypeptide or polynucleotide comprises sequences components G, H, I, J and K of Table 6.

E38. A non-naturally occurring polypeptide or polynucleotide, wherein the polypeptide or polynucleotide comprises sequences of components G, H, I, J, K, L1, L2 and L4 of Table 6.

E39. A non-naturally occurring polypeptide or polynucleotide, wherein the polypeptide or polynucleotide comprises sequences of any two or more of the components I, L, M, or N of Table 6.

E40. A non-naturally occurring polypeptide or polynucleotide, wherein the polypeptide or polynucleotide comprises sequences of any two or more of the components I, L, M, N, L2, L4, L5, L6 or L7 of Table 6.

E41. A non-naturally occurring polypeptide or polynucleotide, wherein the polypeptide or polynucleotide comprises sequences of components I, L, M, and N of Table 6.

E42. A non-naturally occurring polypeptide or polynucleotide, wherein the polypeptide or polynucleotide comprises sequences of components I, L, M, N, L2, L4, L5, L6 and L7 of Table 6.

E43. A non-naturally occurring polypeptide or polynucleotide, wherein the polypeptide or polynucleotide comprises sequences of any two or more of the components N, O, or P of Table 6.

E44. A non-naturally occurring polypeptide or polynucleotide, wherein the polypeptide or polynucleotide comprises sequences of any two or more of the components N, O, P, L2, L3, L4, L5, L6, L7 or L8 of Table 6.

E45. A non-naturally occurring polypeptide or polynucleotide, wherein the polypeptide or polynucleotide comprises sequences of components N, O and P of Table 6.

E46. A non-naturally occurring polypeptide or polynucleotide, wherein the polypeptide or polynucleotide comprises sequences of components N, O, P, L2, L3, L4, L5, L6, L7 and L8 of Table 6.

E47. A non-naturally occurring polypeptide or polynucleotide, wherein the polypeptide or polynucleotide comprises sequences of components A, B, C, D, E, F, and L1 of Table 6, in any order.

E48. A non-naturally occurring polypeptide or polynucleotide, wherein the polypeptide or polynucleotide comprises, in sequential order, sequence components A, L1, B, L1, C, L1, D, L1, E, L1, and F of Table 6.

E49. A non-naturally occurring polypeptide or polynucleotide, wherein the polypeptide or polynucleotide comprises sequences of components A, B, C, D, E, F, L2, L3 and L4 of Table 6, in any order.

E50. A non-naturally occurring polypeptide or polynucleotide, wherein the polypeptide or polynucleotide comprises, in sequential order, sequence components A, L4, B, L3, C, L2, D, L3, E, L4 and F of Table 6.

E51. A non-naturally occurring polypeptide or polynucleotide, wherein the polypeptide or polynucleotide comprises sequences of components G, H, I, J, K, L1, L2 and L4 of Table 6, in any order.

E52. A non-naturally occurring polypeptide or polynucleotide, wherein the polypeptide or polynucleotide comprises, in sequential order, sequence components G, L1, L2, L1, H, L1, L4, L1, I, L1, J, L1 and K of Table 6.

E53. A non-naturally occurring polypeptide or polynucleotide, wherein the polypeptide or polynucleotide comprises sequences of components I, L, M, N, L2, L4, L5, L6 and L7 of Table 6, in any order.

E54. A non-naturally occurring polypeptide or polynucleotide, wherein the polypeptide or polynucleotide comprises, in sequential order, sequence components L, L7, L2, L7, M, L5, L4, L6, I, L7, L2, L7 and N of Table 6.

E55. A non-naturally occurring polypeptide or polynucleotide, wherein the polypeptide or polynucleotide comprises sequences of components N, 0, P, L2, L3, L4, L5, L6, L7 and L8 of Table 6, in any order.

E56. A non-naturally occurring polypeptide or polynucleotide, wherein the polypeptide or polynucleotide comprises, in sequential order, sequence components N, L8, L2, L8, 0, L8, P, L8, L4, L8, L3, L8, L5, L8, L6, L8 and L7 of Table 6.

E57. A polynucleotide or polypeptide in any one of E26 to E56, wherein any one or more LI sequence components are substituted with L9 sequence components of Table 6.

E58. A polypeptide comprising any two or more of the sequences of: AA Subset No. 1, AA Subset No. 2, AA Subset No. 3, AA Subset No. 4, or AA Subset No. 5, wherein the sequences are in any order, or are in sequential order, as shown below.

E59. A polypeptide comprising the sequences of: AA Subset No. 1, AA Subset No. 2, AA Subset No. 3, AA Subset No. 4, or AA Subset No. 5, wherein the sequences are in any order, or are in sequential order, as shown below.

E60. A polynucleotide comprising a nucleic acid sequence encoding a polypeptide comprising any two or more of the sequences of: AA Subset No. 1, AA Subset No. 2, AA Subset No. 3, AA Subset No. 4, or AA Subset No. 5, wherein the sequences are in any order, or are in sequential order, as shown below.

E61. A polynucleotide comprising a nucleic acid sequence encoding a polypeptide comprising the sequences of: AA Subset No. 1, AA Subset No. 2, AA Subset No. 3, AA Subset No. 4, or AA Subset No. 5, wherein the sequences are in any order, or are in sequential order, as shown below.

### AA Subset No. 1:

SEQ ID NO: 1 (HPV6 E2*);
SEQ ID NO: 34 (rigid linker);
SEQ ID NO: 3 (HPV6 E4 1-56);
SEQ ID NO: 34 (rigid linker);
SEQ ID NO: 5 (HPV6 E7 70-98);
SEQ ID NO: 34 (rigid linker);
SEQ ID NO: 7 (HPV6 E4 36-90);
SEQ ID NO: 34 (rigid linker);
SEQ ID NO: 9 (HPV6 E7 1-58);
SEQ ID NO: 34 (rigid linker); and/or,
SEQ ID NO: 11 (HPV6 E6*).

### AA Subset No. 2:

SEQ ID NO: 1 (HPV6 E2*);
SEQ ID NO: 45 (HPV1 1 E7 epitope linker);
SEQ ID NO: 3 (HPV6 E4);
SEQ ID NO: 42 (HPV1 1 E6 linker);
SEQ ID NO: 5 (HPV6 E7);
SEQ ID NO: 40 (HPV6 E6 linker);
SEQ ID NO: 7 (HPV6 E4);
SEQ ID NO: 42 (HPV1 1 E6 linker);
SEQ ID NO: 9 (HPV6 E7);
SEQ ID NO: 45 (HPV1 1 E7 epitope linker); and/or,
SEQ ID NO: 11 (HPV6 E6*).

### AA Subset No. 3:

SEQ ID NO: 13 (HPV6 E2* 1-368 (Del 206-306));
SEQ ID NO: 34 (rigid linker);
SEQ ID NO: 42 (HPV1 1 E6 epitope “linker”);
SEQ ID NO: 34 (rigid linker);
SEQ ID NO: 15 (HPV6 E4 1-70 (Del 21-50));
SEQ ID NO: 34 (rigid linker);
SEQ ID NO: 45 (HPV1 1 E7 epitope “linker”);
SEQ ID NO: 34 (rigid linker);
SEQ ID NO: 17 (HPV6 E4 51-70);
SEQ ID NO: 34 (rigid linker);
SEQ ID NO: 20 (HPV6 E6* 1-120 (Del C-term));
SEQ ID NO: 34 (rigid linker); and/or,
SEQ ID NO: 22 (HPV6 E7 1-98 (Del 40-75)).

### AA Subset No. 4:

SEQ ID NO: 24 (HPV6 E2* 1-205);
SEQ ID NO: 52 (HPV16 E7 agonist enhancer linker);
SEQ ID NO: 42 (HPV11 E6 epitope linker);
SEQ ID NO: 52 (HPV16 E7 agonist enhancer linker);
SEQ ID NO: 26 (HPV6 E4 1-20);
SEQ ID NO: 48 (HPV16 E6 agonist enhancer No. 1 linker);
SEQ ID NO: 45 (HPV11 E7 epitope linker);
SEQ ID NO: 50 (HPV16 E6 agonist enhancer No. 2 linker);
SSEQ ID NO: 17 (HPV6 E4 51-70);
SEQ ID NO: 52 (HPV16 E7 agonist enhancer linker);
SEQ ID NO: 40 (HPV6 E6 epitope linker);
SEQ ID NO: 52 (HPV16 E7 agonist enhancer linker); and/or,
SEQ ID NO: 28 (HPV6 E2* 307-368).

### AA Subset No. 5:

SEQ ID NO: 28 (HPV6 E2*);
SEQ ID NO: 57 (Flex linker);
SEQ ID NO: 40 (HPV6 E6 epitope linker);
SEQ ID NO: 57 (Flex linker);
SEQ ID NO: 30 (HPV6 E4 Epitope—No. 1);
SEQ ID NO: 57 (Flex linker);
SEQ ID NO: 32 (HPV6 E4 Epitope—No. 2);
SEQ ID NO: 57 (Flex linker);
SEQ ID NO: 45 (HPV11 E7 epitope linker);
SEQ ID NO: 57 (Flex linker);
SEQ ID NO: 42 (HPV11 E6 epitope linker);
SEQ ID NO: 57 (Flex linker);
SEQ ID NO: 48 (HPV16 E6 Agonist Enhancer No. 1);
SEQ ID NO: 57 (Flex linker);
SEQ ID NO: 50 (HPV16 E6 Agonist Enhancer No. 2);
SEQ ID NO: 57 (Flex linker); and/or,
SEQ ID NO: 52 (HPV16 E7 agonist enhancer).

E62. A polynucleotide comprising any two or more nucleic acid sequences of: DNA Subset No. 1, DNA Subset No. 2, DNA Subset No. 3, DNA Subset No. 4, or DNA Subset No. 5, wherein the sequences are in any order, or are in sequential order, as shown below.

E63. A polynucleotide comprising nucleic acid sequences of: DNA Subset No. 1, DNA Subset No. 2, DNA Subset No. 3, DNA Subset No. 4, or DNA Subset No. 5, wherein the sequences are in any order, or are in sequential order, as shown below.

### DNA Subset No. 1:

SEQ ID NO: 2 (HPV6 E2*);
SEQ ID NO: 35, 36, 37, 38 or 39 (rigid linker);
SEQ ID NO: 4 (HPV6 E4 1-56);
SEQ ID NO: 35, 36, 37, 38 or 39 (rigid linker);
SEQ ID NO: 6 (HPV6 E7 70-98);
SEQ ID NO: 35, 36, 37, 38 or 39 (rigid linker);
SEQ ID NO: 8 (HPV6 E4 36-90);
SEQ ID NO: 35, 36, 37, 38 or 39 (rigid linker);
SEQ ID NO: 10 (HPV6 E7 1-58);
SEQ ID NO: 35, 36, 37, 38 or 39 (rigid linker); and/or,
SEQ ID NO: 12 (HPV6 E6*).

### DNA Subset No. 2;

SEQ ID NO: 2 (HPV6 E2*);
SEQ ID NO: 46 or 47 (HPV1 1 E7 epitope linker);
SEQ ID NO: 4 (HPV6 E4);
SEQ ID NO: 43 or 44 (HPV1 1 E6 linker);
SEQ ID NO: 6 (HPV6 E7);
SEQ ID NO: 41 (HPV6 E6 linker);
SEQ ID NO: 8 (HPV6 E4);
SEQ ID NO: 43 or 44 (HPV1 1 E6 linker);
SEQ ID NO: 10 (HPV6 E7 1-58);
SEQ ID NO: 46 or 47 (HPV11 E7 epitope linker); and/or,
SEQ ID NO: 12 (HPV6 E6*).

### DNA Subset No. 3;

SEQ ID NO: 14 (HPV6 E2* 1-368 (Del 206-306));
SEQ ID NO: 35, 36, 37, 38 or 39 (rigid linker);
SEQ ID NO: 43 or 44 (HPV1 1 E6 epitope linker);
SEQ ID NO: 35, 36, 37, 38 or 39 (rigid linker);
SEQ ID NO: 16 (HPV6 E4 1-70 (Del 21-50));
SEQ ID NO: 35, 36, 37, 38 or 39 (rigid linker);
SEQ ID NO: 46 or 47 (HPV11 E7 epitope linker);
SEQ ID NO: 35, 36, 37, 38 or 39 (rigid linker);
SEQ ID NO: 18 or 19 (HPV6 E4 51-70);
SEQ ID NO: 35, 36, 37, 38 or 39 (rigid linker);
SEQ ID NO: 21 (HPV6 E6* 1-120 (Del C-term));
SEQ ID NO: 35, 36, 37, 38 or 39 (rigid linker); and/or,
SEQ ID NO: 23 (HPV6 E7 1-98 (Del 40-75)).

### DNA Subset No. 4:

SEQ ID NO: 25 (HPV6 E2* 1-205);
SEQ ID NO: 53, 54, 55, or 56 (HPV16 E7 agonist enhancer linker);
SEQ ID NO: 43 or 44 (HPV11 E6 epitope linker);
SEQ ID NO: 53, 54, 55, or 56 (HPV16 E7 agonist enhancer linker);
SEQ ID NO: 27 (HPV6 E4 1-20);
SEQ ID NO: 49 (HPV16 E6 Agonist Enhancer No. 1 linker);
SEQ ID NO: 46 or 47 (HPV1 1 E7 epitope linker);
SEQ ID NO: 51 (HPV1 6 E6 Agonist Enhancer No. 2 linker);
SEQ ID NO: 18 or 19 (HPV6 E4 51-70);
SEQ ID NO: 53, 54, 55, or 56 (HPV16 E7 agonist enhancer linker);
SEQ ID NO: 41 (HPV6 E6 epitope linker);
SEQ ID NO: 53, 54, 55, or 56 (HPV16 E7 agonist enhancer linker); and/or,
SEQ ID NO: 29 (HPV6 E2* 307-368).

### DNA Subset No. 5:

SEQ ID NO: 29 (HPV6 E2*);
SEQ ID NO: 58, 59, 60, 61, 62, 63, 64 or 65 (Flex linker);
SEQ ID NO: 41 (HPV6 E6 epitope linker);
SEQ ID NO: 58, 59, 60, 61, 62, 63, 64 or 65 (Flex linker);
SEQ ID NO: 31 (HPV6 E4 Epitope-No. 1);
SEQ ID NO: 58, 59, 60, 61, 62, 63, 64 or 65 (Flex linker);
SEQ ID NO: 33 (HPV6 E4 Epitope-No. 2);
SEQ ID NO: 58, 59, 60, 61, 62, 63, 64 or 65 (Flex linker);
SEQ ID NO: 46 or 47 (HPV11 E7 epitope linker);
SEQ ID NO: 58, 59, 60, 61, 62, 63, 64 or 65 (Flex linker);
SEQ ID NO: 43 or 44 (HPV11 E6 epitope linker);
SEQ ID NO: 58, 59, 60, 61, 62, 63, 64 or 65 (Flex linker);
SEQ ID NO: 49 (HPV16 E6 Agonist Enhancer No. 1);
SEQ ID NO: 58, 59, 60, 61, 62, 63, 64 or 65 (Flex linker);
SEQ ID NO: 51 (HPV16 E6 Agonist Enhancer No. 2);
SEQ ID NO: 58, 59, 60, 61, 62, 63, 64 or 65 (Flex linker); and/or,
SEQ ID NO: 53, 54, 55, or 56 (HPV16 E7 agonist enhancer).

E63.1. A polynucleotide comprising a nucleic acid sequence encoding a non-naturally occurring polypeptide comprising an amino acid sequence at least 80% identical to SEQ ID NO: 68.

E63.2. The polynucleotide of E63.1 comprising a nucleic acid sequence encoding a non-naturally occurring polypeptide comprising an amino acid sequence at least 85% identical to SEQ ID NO: 68.

E63.3. The polynucleotide of E63.2 comprising a nucleic acid sequence encoding a non-naturally occurring polypeptide comprising an amino acid sequence at least 90% identical to SEQ ID NO: 68.

E63.4. The polynucleotide of E63.3 comprising a nucleic acid sequence encoding a non-naturally occurring polypeptide comprising an amino acid sequence at least 95% identical to SEQ ID NO: 68.

E63.5. The polynucleotide of E63.4 comprising a nucleic acid sequence encoding the amino acid sequence of SEQ ID NO: 68.

E63.6. The polynucleotide of E63.1, wherein the polynucleotide comprises a nucleic acid sequence at least 80% identical to SEQ ID NO: 116.

E63.7. The polynucleotide of E63.6 comprising a nucleic acid sequence at least 85% identical to SEQ ID NO: 116.

E63.8. The polynucleotide of E63.7 comprising a nucleic acid sequence at least 90% identical to SEQ ID NO: 116.

E63.9. The polynucleotide of E63.8 comprising a nucleic acid sequence at least 95% identical to SEQ ID NO: 116.

E63.10. The polynucleotide of E63.9 comprising the nucleic acid sequence of SEQ ID NO: 116.

E63.11. A polynucleotide comprising a nucleic acid sequence encoding the amino acid sequences of SEQ ID NO: 1, SEQ ID NO: 3, SEQ ID NO: 5, SEQ ID NO: 7, SEQ ID NO: 9, and SEQ ID NO: 11.

E63.12. The polynucleotide of E63.11, wherein the polynucleotide comprises the nucleic acid sequences of SEQ ID NO: 2, SEQ ID NO: 4, SEQ ID NO: 6, SEQ ID NO: 8, SEQ ID NO: 10, and SEQ ID NO: 12.

E63.13. The polynucleotide of E63.11 or E63.12, further comprising a nucleic acid sequence encoding the amino acid sequences of SEQ ID NO: 40, SEQ ID NO: 42, and SEQ ID NO: 45.

E63.14. The polynucleotide of E63.13, wherein the polynucleotide comprises the nucleic acid sequences of SEQ ID NO: 41, SEQ ID NO: 43, and SEQ ID NO: 46.

E63.15. The polynucleotide of E63.13, wherein the polynucleotide comprises the nucleic acid sequences of SEQ ID NO: 41, SEQ ID NO: 43, and SEQ ID NO: 47.

E63.16. The polynucleotide of E63.13, wherein the polynucleotide comprises the nucleic acid sequences of SEQ ID NO: 41, SEQ ID NO: 44, and SEQ ID NO: 46.

E63.17. The polynucleotide of E63.13, wherein the polynucleotide comprises the nucleic acid sequences of SEQ ID NO: 41, SEQ ID NO: 44, and SEQ ID NO: 47.

E63.18. A polynucleotide comprising a nucleic acid sequence encoding a non-naturally occurring polypeptide comprising an amino acid sequence at least 80% identical to SEQ ID NO: 66, SEQ ID NO: 70, SEQ ID NO: 72, or SEQ ID NO: 74.

E63.19. The polynucleotide of claim 18, wherein the polynucleotide comprises a sequence at least 80% identical to SEQ ID NO: 67, SEQ ID NO: 71, SEQ ID NO: 73, or SEQ ID NO: 75.

E63.20. A polynucleotide encoding a non-naturally occurring polypeptide, wherein the non-naturally occurring polypeptide comprises HPV epitopes derived from an HPV6 E2 protein, an EPV6 E4 protein, an HPV6 E6 protein, and HPV6 E7 protein, and HPV11 E6 protein, and an HPV11 E7 protein.

E64. A vector comprising the polynucleotide of any one of E1 to E16 and E26 to E63.20.

E65. The vector of E64, wherein the vector is a plasmid, a viral, or a non-viral vector.

E66. The vector of E65, wherein the viral vector is an adenoviral vector.

E67. The vector of E66, wherein the adenoviral vector is a non-human primate viral vector.

E67.1. The vector of E67, wherein the vector comprises a nucleic acid sequence having at least 90% sequence identity with SEQ ID NO: 119.

E67.2. The vector of E67, wherein the vector comprises a nucleic acid sequence having at least 80%, 85%, 90%, 95%, 96%, 97%, 98%, or 99% sequence identity with SEQ ID NO: 119.

E67.3. The vector of E67, wherein the vector comprises a nucleic acid sequence having a codon degenerate variant of SEQ ID NO: 119.

E68. A composition or vaccine comprising a pharmaceutically acceptable carrier and a polynucleotide in any one of E1 to E16 and E26 to E63.20, a polypeptide of any one of E17 and E26 to E59, or a vector of any one of E18 to E21 and E64 to E67.3.

E69. The polynucleotide, polypeptide, vector, composition or vaccine of any one of E1 to E22 and E26 to E68, wherein the polynucleotide, polypeptide, vector, composition or vaccine is capable of inducing an anti-HPV immune response.

E70. Use of a polynucleotide, polypeptide, vector, composition or vaccine in E69 for treatment of a patient that has an HPV-associated disease or disorder.

E71. The use of a polynucleotide, polypeptide, vector, composition or vaccine in E70, wherein the disease or disorder is an HPV6, HPV11 or HPV16-associated disease or disorder.

E72. The use of a polynucleotide, polypeptide, vector, composition or vaccine in E70, wherein the disease or disorder is recurrent respiratory papillomatosis (RRP), anogenital warts, lower genital tract neoplasia (e.g., cervical, vaginal, and vulvar intraepithelial neoplasia), cervical cancer, vulvar cancer, anal cancer, penile cancer, or head and neck cancers.

E72.1 The use of a polynucleotide, polypeptide, vector, composition or vaccine in E70, wherein the disease or disorder is RRP.

E72.2 The use of a polynucleotide, polypeptide, vector, composition or vaccine in E70, wherein the disease or disorder is anogenital warts.

E72.3 The use of a polynucleotide, polypeptide, vector, composition or vaccine in E70, wherein the disease or disorder is lower genital tract neoplasia.

E72.4 The use of a polynucleotide, polypeptide, vector, composition or vaccine in E70, wherein the disease or disorder is one or more of cervical, vaginal, and vulvar intraepithelial neoplasia.

E72.5 The use of a polynucleotide, polypeptide, vector, composition or vaccine in E70, wherein the disease or disorder is cervical cancer.

E72.6 The use of a polynucleotide, polypeptide, vector, composition or vaccine in E70, wherein the disease or disorder is vulvar cancer.

E72.7 The use of a polynucleotide, polypeptide, vector, composition or vaccine in E70, wherein the disease or disorder is anal cancer.

E72.8 The use of a polynucleotide, polypeptide, vector, composition or vaccine in E70, wherein the disease or disorder is penile cancer.

E72.9 The use of a polynucleotide, polypeptide, vector, composition or vaccine in E70, wherein the disease or disorder is a head and neck cancer.

E73. The use of a polynucleotide, polypeptide, vector, composition or vaccine in E70, wherein the treatment involves treating the patient with one or more additional therapies.

E74. The use of a polynucleotide, polypeptide, vector, composition or vaccine in E73, wherein the one or more additional therapies is a surgical or non-surgical therapy.

E75. The use of a polynucleotide, polypeptide, vector, composition or vaccine in E73, wherein the one or more additional therapies is a surgical therapy.

E76. The use of a polynucleotide, polypeptide, vector, composition or vaccine in E75, wherein the surgical therapy is performed via microscopic or endoscopic rigid laryngoscopy in the operating room using either a laser or microdebrider to remove papillomas.

E77. The use of a polynucleotide, polypeptide, vector, composition or vaccine in E75, wherein the surgical therapy comprises a debulking procedure.

E78. The use of a polynucleotide, polypeptide, vector, composition or vaccine in E76, wherein the surgical therapy comprises a tracheotomy.

E78.1 The use of a polynucleotide, polypeptide, vector, composition or vaccine in E76, wherein the surgical therapy comprises use of a laser or microdebrider.

E79. The use of a polynucleotide, polypeptide, vector, composition or vaccine in E73, wherein the one or more additional therapies is a non-surgical therapy.

E80. The use of a polynucleotide, polypeptide, vector, composition or vaccine in E79, wherein the non-surgical therapy comprises co-administration of an additional agent.

E81. The use of a polynucleotide, polypeptide, vector, composition or vaccine in E80, wherein the additional agent is contained in the same composition that contains the polynucleotide, polypeptide, vector, composition or vaccine.

E82. The use of a polynucleotide, polypeptide, vector, composition or vaccine in E80, wherein the additional agent is a chemotherapy agent, an anti-inflammatory agent, an analgesic, a biological response modifier, a vector comprising such agents, or a cell comprising the agent or a nucleic acid encoding the same.

E83. The use of a polynucleotide, polypeptide, vector, composition or vaccine in E80, wherein the additional agent is administered at or near the same location as the polynucleotide, polypeptide, vector, composition or vaccine.

E84. The use of a polynucleotide, polypeptide, vector, composition or vaccine in E80, wherein the additional agent is administered at a different location than the polynucleotide, polypeptide, vector, composition or vaccine.

E85. The use of a polynucleotide, polypeptide, vector, composition or vaccine in E80, wherein the additional agent is administered simultaneous with the administration of the polynucleotide, polypeptide, vector, composition or vaccine.

E86. The use of a polynucleotide, polypeptide, vector, composition or vaccine in E80, wherein the additional agent is administered at a different time than administration of the polynucleotide, polypeptide, vector, composition or vaccine.

E87. The use of a polynucleotide, polypeptide, vector, composition or vaccine in E80, wherein the additional agent is selected from the group consisting of: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

E88. The use of a polynucleotide, polypeptide, vector, composition or vaccine in E80, wherein the additional agent is selected from the group consisting of: acetaminophen, oxycodone, tramadol, and proporxyphene hydrochloride.

E89. The use of a polynucleotide, polypeptide, vector, composition or vaccine in E80, wherein the additional agent is a molecule directed against cell surface markers (e.g., CD4, CD5, etc.).

E90. The use of a polynucleotide, polypeptide, vector, composition or vaccine in E80, wherein the additional agent is a cytokine inhibitor, such as a TNF inhibitor (e.g., etanercept (ENBREL®), adalimumab (HUMIRA®), and infliximab (REMICADE®)).

E91. The use of a polynucleotide, polypeptide, vector, composition or vaccine in E80, wherein the additional agent is a nucleotide analogs (e.g., Cidofovir).

E92. The use of a polynucleotide, polypeptide, vector, composition or vaccine in E80, wherein the additional agent is an angiogenesis inhibitor, such as Bevacizumab (AVASTIN®).

E93. The use of a polynucleotide, polypeptide, vector, composition or vaccine in E80, wherein the additional agent is a non-steroidal anti-inflammatory compound, such as COX-2-selective drugs (e.g., Celexecob (CELEBREX®)).

E94. The use of a polynucleotide, polypeptide, vector, composition or vaccine in E80, wherein the additional agent is an immune checkpoint inhibitor, such as a PD-1 inhibitor (e.g., Pembrolizumab (KEYTRUDA®), Nivolumab (OPDIVO®), and Cemiplimab (LIBTAYO®)) and/or a PD-L1 inhibitor (e.g., Atezolizumab (TECENTRIQ®), Avelumab (BAVENCIO®), and Durvalumab (IMFINZI®)).

E95. The use of a polynucleotide, polypeptide, vector, composition or vaccine in E80, wherein the additional agent is an adhesion molecule inhibitor.

E96. The use of a polynucleotide, polypeptide, vector, composition or vaccine in E80, wherein the additional agent is a monoclonal antibody.

E97. The use of a polynucleotide, polypeptide, vector, composition or vaccine in E80, wherein the additional agent is an anti-rheumatic drug, such as cyclophosphamide, cyclosporine, methotrexate, penicillamine, leflunomide, sulfasalazine, hydroxychloroquine, Gold (oral (auranofin) and intramuscular), and/or minocycline.

E98. The use of a polynucleotide, polypeptide, vector, composition or vaccine in E80, wherein the additional agent is a colony-stimulating factor, such as macrophage colony-stimulating factor, granulocyte macrophage colony-stimulating factor (GM-CSF), granulocyte colony-stimulating factor (G-CSF) or promegapoietin.

E99. The use of a polynucleotide, polypeptide, vector, composition or vaccine in E80, wherein the additional agent is a tumor necrosis factor, such as TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL)).

E100. The use of a polynucleotide, polypeptide, vector, composition or vaccine in E80, wherein the additional agent is an interleukin, such as IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and/or IL-36.

E100. The use of a polynucleotide, polypeptide, vector, composition or vaccine in E80, wherein the additional agent is an interferon, such as IFN-α, IFN-β, IFN-ε, IFN-κ, and IFN-ω, interferon type II, IFN-γ, interferon type III, IFNAI, IFNA2, IFNA4, IFNA5, IFNA6, IFNA7, IFNA8, IFNA10, IFNA13, IFNA14, IFNA16, IFNA17, and/or IFNA21.

E101. The use of a polynucleotide, polypeptide, vector, composition or vaccine in E80, wherein the additional agent is a chemokine selected from the group consisting of: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL1 7; the XC subfamily: XCLI and XCL2; and the CX3C subfamily CX3CL1.

E102. The use of a polynucleotide, polypeptide, vector, composition or vaccine in E80, wherein the additional agent is a membrane-bound cytokine, which is co-expressed with a chimeric antigen receptor (CAR).

E103. The use of a polynucleotide, polypeptide, vector, composition or vaccine in E80, wherein the additional agent is a cytokine selected from IL2, IL7, IL12, IL15, a fusion of IL-15 and IL-15Rα, IL21, IFNγ or TNF-α.

E104. The use of a polynucleotide, polypeptide, vector, composition or vaccine in E80, wherein administration of the polynucleotide, polypeptide, vector, composition or vaccine in combination with the additional agent enhances the treatment of a disease or disorder and/or reduces any side-effects of treatment with the polynucleotide, polypeptide, vector, composition or vaccine alone.

E105. The use of a polynucleotide, polypeptide, vector, composition or vaccine in E80, wherein the disease being treated is RRP.

E106. A polynucleotide, polypeptide, vector, composition or vaccine in E69 for use in a method of treating a patient that has an HPV-associated disease or disorder.

E107. The polynucleotide, polypeptide, vector, composition or vaccine in E69 for use in a method of treating a patient that has an HPV-associated disease or disorder, wherein the disease or disorder is an HPV6, HPV11 or HPV16-associated disease or disorder.

E108. The polynucleotide, polypeptide, vector, composition or vaccine in E69 for use in a method of treating a patient that has an HPV-associated disease or disorder, wherein the disease or disorder is recurrent respiratory papillomatosis (RRP), anogenital warts, lower genital tract neoplasia (e.g., cervical, vaginal, and vulvar intraepithelial neoplasia), cervical cancer, vulvar cancer, anal cancer, penile cancer, or head and neck cancers.

E108.1 The polynucleotide, polypeptide, vector, composition or vaccine in E69 for use in a method of treating a patient that has an HPV-associated disease or disorder, wherein the disease or disorder is RRP.

E108.2 The polynucleotide, polypeptide, vector, composition or vaccine in E69 for use in a method of treating a patient that has an HPV-associated disease or disorder, wherein the disease or disorder is anogenital warts.

E108.3 The polynucleotide, polypeptide, vector, composition or vaccine in E69 for use in a method of treating a patient that has an HPV-associated disease or disorder, wherein the disease or disorder is lower genital tract neoplasia.

E108.4 The polynucleotide, polypeptide, vector, composition or vaccine in E69 for use in a method of treating a patient that has an HPV-associated disease or disorder, wherein the disease or disorder is one or more of cervical, vaginal, and vulvar intraepithelial neoplasia.

E108.5 The polynucleotide, polypeptide, vector, composition or vaccine in E69 for use in a method of treating a patient that has an HPV-associated disease or disorder, wherein the disease or disorder is cervical cancer.

E108.6 The polynucleotide, polypeptide, vector, composition or vaccine in E69 for use in a method of treating a patient that has an HPV-associated disease or disorder, wherein the disease or disorder is vulvar cancer.

E108.7 The polynucleotide, polypeptide, vector, composition or vaccine in E69 for use in a method of treating a patient that has an HPV-associated disease or disorder, wherein the disease or disorder is anal cancer.

E108.8 The polynucleotide, polypeptide, vector, composition or vaccine in E69 for use in a method of treating a patient that has an HPV-associated disease or disorder, wherein the disease or disorder is penile cancer.

E108.9 The polynucleotide, polypeptide, vector, composition or vaccine in E69 for use in a method of treating a patient that has an HPV-associated disease or disorder, wherein the disease or disorder is a head and neck cancer.

E109. The polynucleotide, polypeptide, vector, composition or vaccine in E69 for use in a method of treating a patient that has an HPV-associated disease or disorder, wherein the treatment involves treating the patient with one or more additional therapies.

E110. The polynucleotide, polypeptide, vector, composition or vaccine in E109 for use in a method of treating a patient that has an HPV-associated disease or disorder, wherein the one or more additional therapies is a surgical or non-surgical therapy.

E111. The polynucleotide, polypeptide, vector, composition or vaccine in E110 for use in a method of treating a patient that has an HPV-associated disease or disorder, wherein the one or more additional therapies is a surgical therapy.

E112. The polynucleotide, polypeptide, vector, composition or vaccine in E111 for use in a method of treating a patient that has an HPV-associated disease or disorder, wherein the surgical therapy is performed via microscopic or endoscopic rigid laryngoscopy in the operating room using either a laser or microdebrider to remove papillomas.

E113. The polynucleotide, polypeptide, vector, composition or vaccine in E111 for use in a method of treating a patient that has an HPV-associated disease or disorder, wherein the surgical therapy comprises a debulking procedure.

E114. The polynucleotide, polypeptide, vector, composition or vaccine in E111 for use in a method of treating a patient that has an HPV-associated disease or disorder, wherein the surgical therapy comprises a tracheotomy.

E114.1 The polynucleotide, polypeptide, vector, composition or vaccine in E111 for use in a method of treating a patient that has an HPV-associated disease or disorder, wherein the surgical therapy comprises use of a laser or microdebrider.

E115. The polynucleotide, polypeptide, vector, composition or vaccine in E109 for use in a method of treating a patient that has an HPV-associated disease or disorder, wherein the one or more additional therapies is a non-surgical therapy.

E116. The polynucleotide, polypeptide, vector, composition or vaccine in E115 for use in a method of treating a patient that has an HPV-associated disease or disorder, wherein the non-surgical therapy comprises co-administration of an additional agent.

E117. The polynucleotide, polypeptide, vector, composition or vaccine in E116 for use in a method of treating a patient that has an HPV-associated disease or disorder, wherein the additional agent is contained in the same composition that contains the polynucleotide, polypeptide, vector, composition or vaccine.

E118. The polynucleotide, polypeptide, vector, composition or vaccine in E116 for use in a method of treating a patient that has an HPV-associated disease or disorder, wherein the additional agent is a chemotherapy agent, an anti-inflammatory agent, an analgesic, a biological response modifier, a vector comprising such agents, or a cell comprising the agent or a nucleic acid encoding the same.

E119. The polynucleotide, polypeptide, vector, composition or vaccine in E116 for use in a method of treating a patient that has an HPV-associated disease or disorder, wherein the additional agent is administered at or near the same location as the polynucleotide, polypeptide, vector, composition or vaccine.

E120. The polynucleotide, polypeptide, vector, composition or vaccine in E116 for use in a method of treating a patient that has an HPV-associated disease or disorder, wherein the additional agent is administered at a different location than the polynucleotide, polypeptide, vector, composition or vaccine.

E121. The polynucleotide, polypeptide, vector, composition or vaccine in E116 for use in a method of treating a patient that has an HPV-associated disease or disorder, wherein the additional agent is administered simultaneous with the administration of the polynucleotide, polypeptide, vector, composition or vaccine.

E122. The polynucleotide, polypeptide, vector, composition or vaccine in E116 for use in a method of treating a patient that has an HPV-associated disease or disorder, wherein the additional agent is administered at a different time than administration of the polynucleotide, polypeptide, vector, composition or vaccine.

E123. The polynucleotide, polypeptide, vector, composition or vaccine in E116 for use in a method of treating a patient that has an HPV-associated disease or disorder, wherein the additional agent is selected from the group consisting of: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

E124. The polynucleotide, polypeptide, vector, composition or vaccine in E116 for use in a method of treating a patient that has an HPV-associated disease or disorder, wherein the additional agent is selected from the group consisting of: acetaminophen, oxycodone, tramadol, and proporxyphene hydrochloride.

E125. The polynucleotide, polypeptide, vector, composition or vaccine in E116 for use in a method of treating a patient that has an HPV-associated disease or disorder, wherein the additional agent is a molecule directed against cell surface markers (e.g., CD4, CD5, etc.).

E126. The polynucleotide, polypeptide, vector, composition or vaccine in E116 for use in a method of treating a patient that has an HPV-associated disease or disorder, wherein the additional agent is a cytokine inhibitor, such as a TNF inhibitor (e.g., etanercept (ENBREL®), adalimumab (HUMIRA®), and infliximab (REMICADE®)).

E127. The polynucleotide, polypeptide, vector, composition or vaccine in E116 for use in a method of treating a patient that has an HPV-associated disease or disorder, wherein the additional agent is a nucleotide analogs (e.g., Cidofovir).

E128. The polynucleotide, polypeptide, vector, composition or vaccine in E116 for use in a method of treating a patient that has an HPV-associated disease or disorder, wherein the additional agent is an angiogenesis inhibitor, such as Bevacizumab (AVASTIN®).

E129. The polynucleotide, polypeptide, vector, composition or vaccine in E116 for use in a method of treating a patient that has an HPV-associated disease or disorder, wherein the additional agent is a non-steroidal anti-inflammatory compound, such as COX-2-selective drugs (e.g., Celexecob (CELEBREX®)).

E130. The polynucleotide, polypeptide, vector, composition or vaccine in E116 for use in a method of treating a patient that has an HPV-associated disease or disorder, wherein the additional agent is an immune checkpoint inhibitor, such as a PD-1 inhibitor (e.g., Pembrolizumab (KEYTRUDA®), Nivolumab (OPDIVO®), and Cemiplimab (LIBTAYO®)) and/or a PD-L1 inhibitor (e.g., Atezolizumab (TECENTRIQ®), Avelumab (BAVENCIO®), and Durvalumab (IMFINZI®)).

E131. The polynucleotide, polypeptide, vector, composition or vaccine in E116 for use in a method of treating a patient that has an HPV-associated disease or disorder, wherein the additional agent is an adhesion molecule inhibitor.

E132. The polynucleotide, polypeptide, vector, composition or vaccine in E116 for use in a method of treating a patient that has an HPV-associated disease or disorder, wherein the additional agent is a monoclonal antibody.

E133. The polynucleotide, polypeptide, vector, composition or vaccine in E116 for use in a method of treating a patient that has an HPV-associated disease or disorder, wherein the additional agent is an anti-rheumatic drug, such as cyclophosphamide, cyclosporine, methotrexate, penicillamine, leflunomide, sulfasalazine, hydroxychloroquine, Gold (oral (auranofin) and intramuscular), and/or minocycline.

E134. The polynucleotide, polypeptide, vector, composition or vaccine in E116 for use in a method of treating a patient that has an HPV-associated disease or disorder, wherein the additional agent is a colony-stimulating factor, such as macrophage colony-stimulating factor, granulocyte macrophage colony-stimulating factor (GM-CSF), granulocyte colony-stimulating factor (G-CSF) or promegapoietin.

E135. The polynucleotide, polypeptide, vector, composition or vaccine in E116 for use in a method of treating a patient that has an HPV-associated disease or disorder, wherein the additional agent is a tumor necrosis factor, such as TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL)).

E136. The polynucleotide, polypeptide, vector, composition or vaccine in E116 for use in a method of treating a patient that has an HPV-associated disease or disorder, wherein the additional agent is an interleukin, such as IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and/or IL-36.

E137. The polynucleotide, polypeptide, vector, composition or vaccine in E116 for use in a method of treating a patient that has an HPV-associated disease or disorder, wherein the additional agent is an interferon, such as IFN-α, IFN-β, IFN-ε, IFN-κ, and IFN-ω, interferon type II, IFN-γ, interferon type III, IFNAI, IFNA2, IFNA4, IFNA5, IFNA6, IFNA7, IFNA8, IFNA10, IFNA13, IFNA14, IFNA16, IFNA17, and/or IFNA21.

E138. The polynucleotide, polypeptide, vector, composition or vaccine in E116 for use in a method of treating a patient that has an HPV-associated disease or disorder, wherein the additional agent is a chemokine selected from the group consisting of: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL1 7; the XC subfamily: XCLI and XCL2; and the CX3C subfamily CX3CL1.

E139. The polynucleotide, polypeptide, vector, composition or vaccine in E116 for use in a method of treating a patient that has an HPV-associated disease or disorder, wherein the additional agent is a membrane-bound cytokine, which is co-expressed with a chimeric antigen receptor (CAR).

E140. The use of a polynucleotide, polypeptide, vector, composition or vaccine in E80, wherein the additional agent is a cytokine selected from IL2, IL7, IL12, IL15, a fusion of IL-15 and IL-15Rα, IL21, IFNγ or TNF-α.

E141. The polynucleotide, polypeptide, vector, composition or vaccine in E116 for use in a method of treating a patient that has an HPV-associated disease or disorder, wherein administration of the polynucleotide, polypeptide, vector, composition or vaccine in combination with the additional agent enhances the treatment of a disease or disorder and/or reduces any side-effects of treatment with the polynucleotide, polypeptide, vector, composition or vaccine alone.

E142. The polynucleotide, polypeptide, vector, composition or vaccine in E116 for use in a method of treating a patient that has an HPV-associated disease or disorder, wherein the disease being treated is RRP.

E143. A method of treating a patient that has an HPV-associated disease or disorder comprising administering the polynucleotide, polypeptide, vector, composition or vaccine in E69.

E144. The method of treatment of E143, wherein the disease or disorder is an HPV6, HPV11 or HPV16-associated disease or disorder.

E145. The method of treatment of E143, wherein the disease or disorder is recurrent respiratory papillomatosis (RRP), anogenital warts, lower genital tract neoplasia (e.g., cervical, vaginal, and vulvar intraepithelial neoplasia), cervical cancer, vulvar cancer, anal cancer, penile cancer, or head and neck cancers.

E145.1 The method of treatment of E143, wherein the disease or disorder is RRP.

E145.2 The method of treatment of E143, wherein the disease or disorder is anogenital warts.

E145.3 The method of treatment of E143, wherein the disease or disorder is lower genital tract neoplasia.

E145.4 The method of treatment of E143, wherein the disease or disorder is one or more of cervical, vaginal, and vulvar intraepithelial neoplasia.

E145.5 The method of treatment of E143, wherein the disease or disorder is cervical cancer.

E145.6 The method of treatment of E143, wherein the disease or disorder is vulvar cancer.

E145.7 The method of treatment of E143, wherein the disease or disorder is anal cancer.

E145.8 The method of treatment of E143, wherein the disease or disorder is penile cancer.

E145.9 The method of treatment of E143, wherein the disease or disorder is a head and neck cancer.

E146. The method of treatment of E143, further comprising treating the patient with one or more additional therapies.

E147. The method of treatment of E146, wherein the one or more additional therapies is a surgical or non-surgical therapy.

E148. The method of treatment of E146, wherein the one or more additional therapies is a surgical therapy.

E149. The method of treatment of E148, wherein the surgical therapy is performed via microscopic or endoscopic rigid laryngoscopy in the operating room using either a laser or microdebrider to remove papillomas.

E150. The method of treatment of E148, wherein the surgical therapy comprises a debulking procedure.

E150.1. The method of treatment of E150, wherein the debulking is by means of debridement, angiolytic laser, cryotherapy, or carbon dioxide laser.

E150.2. The method of treatment of E150, wherein the debulking is preceded by administration of the polynucleotide, polypeptide, vector, composition or vaccine, either alone or in combination with another therapeutic agent.

E150.3. The method of treatment of E150, wherein the debulking is followed by administration of the polynucleotide, polypeptide, vector, composition or vaccine, either alone or in combination with another therapeutic agent.

E150.4. The method of treatment of E150, wherein the treatment reduces and/or eliminates the need for additional surgical debulking.

E151. The method of treatment of E148, wherein the surgical therapy comprises a tracheotomy.

E151.1 The method of treatment of E148, wherein the surgical therapy comprises use of a laser or microdebrider.

E152. The method of treatment of E146, wherein the one or more additional therapies is a non-surgical therapy.

E153. The method of treatment of E152, wherein the non-surgical therapy comprises co-administration of an additional agent.

E154. The method of treatment of E153, wherein the additional agent is contained in the same composition that contains the polynucleotide, polypeptide, vector, composition or vaccineor or com.

E155. The method of treatment of E153, wherein the additional agent is a chemotherapy agent, an anti-inflammatory agent, an analgesic, a biological response modifier, a vector comprising such agents, or a cell comprising the agent or a nucleic acid encoding the same.

E156. The method of treatment of E153, wherein the additional agent is administered at or near the same location as the polynucleotide, polypeptide, vector, composition or vaccine.

E157. The method of treatment of E153, wherein the additional agent is administered at a different location than the polynucleotide, polypeptide, vector, composition or vaccine.

E158. The method of treatment of E153, wherein the additional agent is administered simultaneous with the administration of the polynucleotide, polypeptide, vector, composition or vaccine.

E159. The method of treatment of E153, wherein the additional agent is administered at a different time than administration of the polynucleotide, polypeptide, vector, composition or vaccine.

E160. The method of treatment of E153, wherein the additional agent is selected from the group consisting of: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

E161. The method of treatment of E153, wherein the additional agent is selected from the group consisting of: acetaminophen, oxycodone, tramadol, and proporxyphene hydrochloride.

E162. The method of treatment of E153, wherein the additional agent is a molecule directed against cell surface markers (e.g., CD4, CD5, etc.).

E163. The method of treatment of E153, wherein the additional agent is a cytokine inhibitor, such as a TNF inhibitor (e.g., etanercept (ENBREL®), adalimumab (HUMIRA®), and infliximab (REMICADE®)).

E164. The method of treatment of E153, wherein the additional agent is a nucleotide analogs (e.g., Cidofovir).

E165. The method of treatment of E153, wherein the additional agent is an angiogenesis inhibitor, such as Bevacizumab (AVASTIN®).

E166. The method of treatment of E153, wherein the additional agent is a non-steroidal anti-inflammatory compound, such as COX-2-selective drugs (e.g., Celexecob (CELEBREX®)).

E167. The method of treatment of E153, wherein the additional agent is an immune checkpoint inhibitor, such as a PD-1 inhibitor (e.g., Pembrolizumab (KEYTRUDA®), Nivolumab (OPDIVO®), and Cemiplimab (LIBTAYO®)) and/or a PD-L1 inhibitor (e.g., Atezolizumab (TECENTRIQ®), Avelumab (BAVENCIO®), and Durvalumab (IMFINZI®)).

E168. The method of treatment of E153, wherein the additional agent is an adhesion molecule inhibitor.

E169. The method of treatment of E153, wherein the additional agent is a monoclonal antibody.

E170. The method of treatment of E153, wherein the additional agent is an anti-rheumatic drug, such as cyclophosphamide, cyclosporine, methotrexate, penicillamine, leflunomide, sulfasalazine, hydroxychloroquine, Gold (oral (auranofin) and intramuscular), and/or minocycline.

E171. The method of treatment of E153, wherein the additional agent is a colony-stimulating factor, such as macrophage colony-stimulating factor, granulocyte macrophage colony-stimulating factor (GM-CSF), granulocyte colony-stimulating factor (G-CSF) or promegapoietin.

E172. The method of treatment of E153, wherein the additional agent is a tumor necrosis factor, such as TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL)).

E173. The method of treatment of E153, wherein the additional agent is an interleukin, such as IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and/or IL-36.

E174. The method of treatment of E153, wherein the additional agent is an interferon, such as IFN-α, IFN-β, IFN-ε, IFN-κ, and IFN-ω, interferon type II, IFN-γ, interferon type III, IFNAI, IFNA2, IFNA4, IFNA5, IFNA6, IFNA7, IFNA8, IFNA10, IFNA13, IFNA14, IFNA16, IFNA17, and/or IFNA21.

E175. The method of treatment of E153, wherein the additional agent is a chemokine selected from the group consisting of: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily: XCL1 and XCL2; and the CX3C subfamily CX3CL1.

E176. The method of treatment of E153, wherein the additional agent is a membrane-bound cytokine, which is co-expressed with a chimeric antigen receptor (CAR).

E177 The method of treatment of E153, wherein the additional agent is a cytokine selected from IL2, IL7, IL12, IL15, a fusion of IL-15 and IL-15Rα, IL21, IFNγ or TNF-α.

E178. The method of treatment of E143, wherein administration of the polynucleotide, polypeptide, vector, composition or vaccine in combination with the additional agent enhances the treatment of a disease or disorder and/or reduces any side-effects of treatment with the polynucleotide, polypeptide, vector, composition or vaccine alone.

E179. The method of treatment of E143, wherein the disease being treated is RRP.

E180. A method of treating a patient that has an HPV-associated disease or disorder comprising administering the vector of any one of E6 thru E67.3.

E181. The method of treatment of E180, wherein the patient is administered a dose of between about 1×10¹¹ and about 5×10¹¹ particle units (PU) of vector.

E182. The method of treatment of E180, wherein the patient is administered about 5×10¹¹ particle units (PU) of vector.

E183. The method of treatment of E180, wherein the vector is administered as adjuvant therapy after standard-of-care debulking surgery.

E184. The method of treatment of E180, wherein the disease being treated is RRP.

E185. The method of treatment of any one of E180 thru 184, wherein the patient is administered one or more doses of vector.

E185.1 The method of treatment of E185, where the doses are administered via injection.

E186. The method of treatment of E185, wherein the patient is administered two doses of vector.

E187. The method of treatment of E185, wherein the patient is administered three doses of vector.

E188. The method of treatment of E185, wherein the patient is administered four doses of vector.

E189. The method of treatment of E185, wherein the patient is administered more than four doses of vector.

E190. The method of treatment of E185, wherein the at least one dose is not accompanied by surgery.

E191. The method of treatment of E185, wherein the patient has no recurrence of papillomatous disease.

E192. The method of treatment of E190, wherein the last dose is administered without surgery.

E193. The method of treatment of E185, wherein the patient is administered up to four doses in separate limbs.

E194. The method of treatment of E185, wherein the minimum time between administrations of the vector is 11 days.

E195. The method of treatment of E185, wherein the patient is administered escalating doses of the vector.

E196. The method of treatment of E185, wherein one or more doses of vector is 5×10¹¹ PU.

E197. The method of treatment of E185.1, wherein the volume for each injection is about 1.0 mL.

E198. The method of treatment of E185, wherein the patient's vital signs are measured within about 30 minutes before and about 30 minutes following administration of the vector.

E199. The method of treatment of E185, wherein the patient is observed for at least one hour after administration of the vector and, if no adverse reactions are observed, the patient is observed for less than one hour for after any subsequent doses.

E200. The method of treatment of E185, wherein the patient undergoes a leukapheresis about six weeks following the final dose of the vector.

E201. The method of treatment of E185, wherein the patient is administered 5×10¹¹ PU of vector on days 1, 15, 43, and 85.

E202. The method of treatment of any one of E180 thru E189, E191, and E193 thru E201, wherein one or more debulking procedures are performed before the last dose is administered.

E203. The method of treatment of E202, wherein one or more debulking procedures are performed after the first dose is administered and before the last dose is administered.

E203.1. The method of treatment of E202, wherein one or more debulking procedures are performed during the administration of at least one dose.

E203.2. The method of treatment of E202, wherein one or more debulking procedures are performed at least 6 months, 1 year, or 2 years following administration of at least one dose.

E203.3. The method of treatment of E203.2, wherein at least one dose is administered following at least one debulking procedure.

E204. The method of treatment of any of E180 thru E203.3, wherein clinical efficacy of the treatment is measured after the final dose is administered.

E205. A method of generating a polynucleotide encoding the polypeptide of E63.5, the method comprising engineering HPV polynucleotide sequences to encode derivatives of naturally occurring HPV polypeptides, wherein at least one of the viral polypeptides has oncogenic biologic activity or an essential viral replication function, wherein a combination of one or more amino acid sequence substitutions, deletions, insertions, separations, or rearrangements are expressed via the composition, vaccine or encoded by the polynucleotide to generate derivative viral polypeptides, wherein HPV oncogenic biologic activity or essential HPV replication function is eliminated from the derivative viral polypeptides, but wherein host immune-responsiveness and/or immunogenic activity against the derivative viral polypeptides is retained or enhanced compared to the corresponding naturally occurring viral polypeptides.

E206. The method of E205, wherein the polynucleotide or derivative viral polypeptide is designed utilizing steps comprising identification of viral antigens combined with any two or more of: in silico prediction of in vivo antigen epitope recognition, in silico prediction of in vivo allergen recognition, human proteome cross-reactivity sequence homology analysis, amino acid sequence physiochemical property analysis, three-dimensional (3D) structure analysis, identification of oncogenic and/or viral function inactivation mutations, deletions, substitutions and/or rearrangements.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims.

### EXAMPLES

The following examples are included to demonstrate preferred embodiments of the invention, in addition to those embodiments disclosed earlier herein. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Manufacture of AdV-HPV6/11 Drug Substance

This example describes a method of manufacturing a DNA vaccine for inducing HPV-specific T lymphocyte responses.

AdV-HPV6/11 is a DNA vaccine comprising a non-human primate adenovector engineered to express a multi-epitope modular fusion protein/antigen for the purpose of inducing a cell-mediated immune response against cells infected with human papillomavirus HPV-6 and HPV-11 serotypes.

AdV-HPV6/11 was developed from the base gorilla adenoviral vector GC46, which was isolated from a healthy African gorilla stool. The GC46 vector is replication incompetent due to deletion of E1 and E4 genes. See FIG. 1A.

The deletion of the E1 region includes base pairs 459 through 3411, resulting in deletion of the E1A and E1B promoters and open reading frames. The E4 region deletion (inclusive of 34144 to 36824) removes all the E4 open reading frames (ORFs), therefore eliminating essential elements for GC46 replication. GC46 E1- and E4-deleted vectors were evaluated in human cell lines for growth, and were found to only replicate in our E1 and E4 complementing cell lines, ORF6 and M2A.

In addition to the E1 and E4 region deletions, the Bovine Growth Hormone polyadenylation (BGH poly A) signal sequence was inserted in place of the E4 open reading frames (ORFs) and provides a solution for the low level of production seen with E4-deleted adenovectors without a spacer. The BGH poly A was inserted at the location of the E4 deletion to stop the transcription initiated from the E4 promoter, which is retained in the GC46 E1- and E4-deleted adenovector.

GC46 was further engineered to express a human papilloma virus 6/11 antigen under the control of a cytomegalovirus (CMV) immediate early promoter driven expression cassette. See FIG. 1B.

The CMV-HPV6/11 antigen expression cassette, located at the E1 region deletion junction, is right-to-left, with respect to the GC46 viral genome. The CMV enhancer/promoter controls the initiation of transcription and within this promoter sequence are the viral enhancer CAAT box, TATA box, transcription start site, and 5′ splice site sequences. The expression cassette sequences are followed by an artificial untranslated region (UTR) containing a splice donor sequence, 3′ splice site sequences, the open reading frame of the gene to be expressed followed by the Simian Virus 40 (SV40) early polyadenylation and transcriptional termination signal sequences. The Tet responsive element (TRE, 42 bp of the Tet operator) is positioned between the TATA box and the transcription initiation site. When this adenovector is produced in cell lines that express the Tet repressor, expression of the CMV driven transgene is reduced. See Gall et al., “Rescue and Production of Vaccine and Therapeutic Adenovirus Vectors Expressing Inhibitory Transgenes.” Molecular Biotechnology. 2007, 35: 263-273. In the absence of tetracycline, the Tet repressor interacts with the TRE element and blocks the initiation of transcription. Upon infection of producer cells that do not express the Tet repressor, normal CMV expression levels are observed.

The protein/antigen expressed from AdV-HPV6/11 (see FIG. 1C) is a fusion of selected regions of HPV proteins that are expressed in HPV-6 and HPV-11 infected cells (HPV-E2, HPV-E4, HPV-E6 and HPV-E7). Selection of the viral protein regions, the design of the module fusion was guided by bioinformatics analyses, immune-informatics techniques, and protein engineering and alterations in protein sequence were made to remove viral protein functionality.

As shown in FIG. 1C, the regions of the AdV-HPV6/11 antigen, described from amino- to carboxyl-terminus, are as follows:
HPV E2 region contains 368 amino acids of the HPV-6 E2 protein, that shares similarity with HPV-11 E2 protein
HPV-11 E7 region contains 15 amino acids of the HPV-11 E7 protein
HPV E4 region contains 56 amino acids of the HPV-6 E4 protein that shares similarity with HPV-11 E4 protein
HPV-11 E6 region contains 15 amino acids of the HPV-11 E6 protein
HPV-6 E7 region contains 29 amino acids of the HPV-6 E7 protein
HPV-6 E6 region contains 15 amino acids of the HPV-6 E6 protein
HPV E4 region contains 55 amino acids of the HPV-6 E4 protein that shares similarity with HPV-11 E4 protein
HPV-11 E6 region contains 15 amino acids of the HPV-11 E6 protein
HPV E7 region contains 58 amino acids of the HPV-6 E7 protein that shares similarity with the HPV-11 E7 protein
HPV-11 E7 region contains 15 amino acids of the HPV-11 E7 protein
HPV E6 region contains 150 amino acids of the HPV-6 E6 protein that shares similarity with the HPV-11 E6 protein

### Example 2: Pre-Clinical Induction of HPV-Specific T Lymphocyte Responses by AdV-HPV6/11 in Patients with RRP

This example demonstrates a method for pre-clinical induction of HPV-specific T lymphocyte responses in patients with recurrent respiratory papillomatosis (RRP).

The ability of AdV-HPV6/11 to induce HPV-specific responses has been studied pre-clinically. First, autologous cultured dendritic cells from three participants with RRP (two with HPV 6 and one with HPV11 disease) were exposed to the base gorilla adenovirus construct encoding GFP (5×10³ MOI), and fluorescence was verified 24 hours after transduction via flow cytometry.

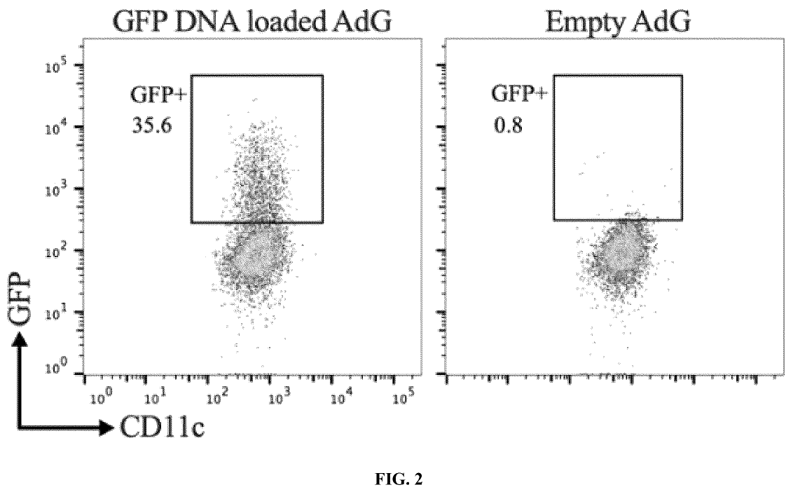

This treatment resulted in strong fluorescent activity in 30-50% of treated dendritic cells. See FIG. 2. This suggests that the adenoviral vector can deliver the nucleic acid payload to dendritic cells. Next, dendritic cells exposed to AdV-HPV6/11 or an empty gorilla adenovirus vector for 24 hours were used to perform three rounds of in vitro stimulation of sorted T lymphocytes from PBMC of three participants with RRP, and positive reactivity was assessed by IFNg ELISA.

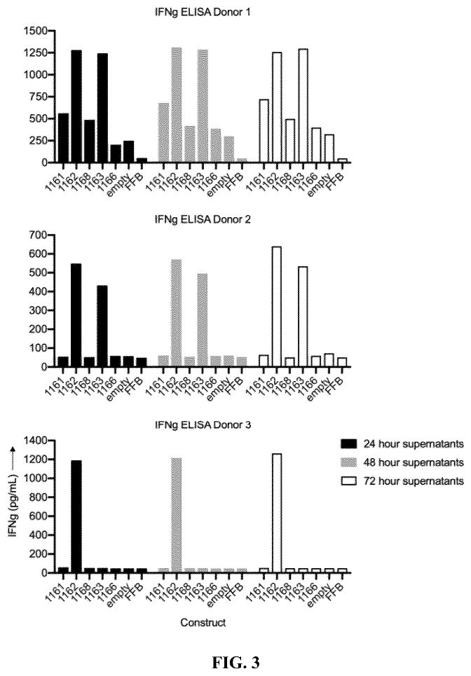

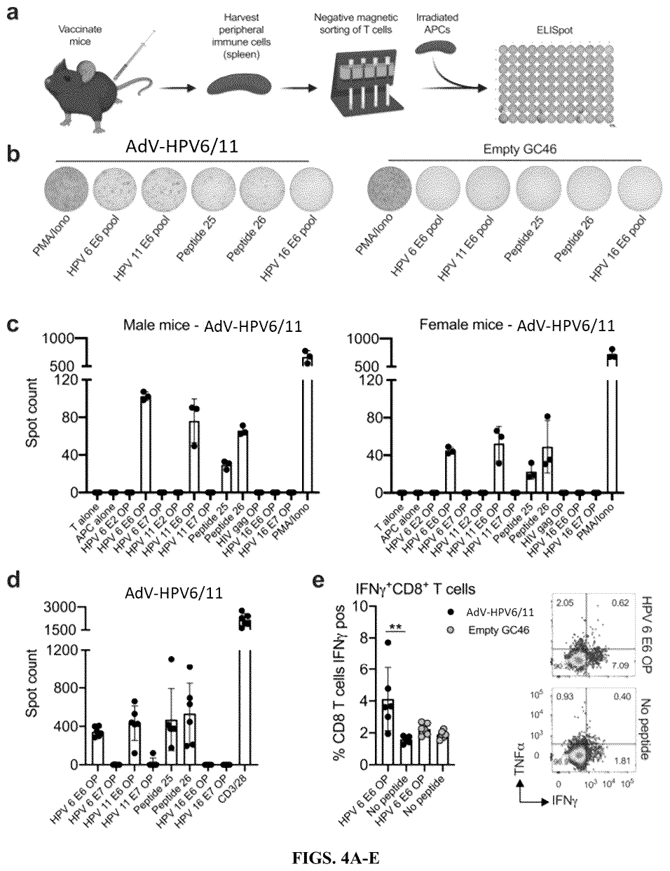

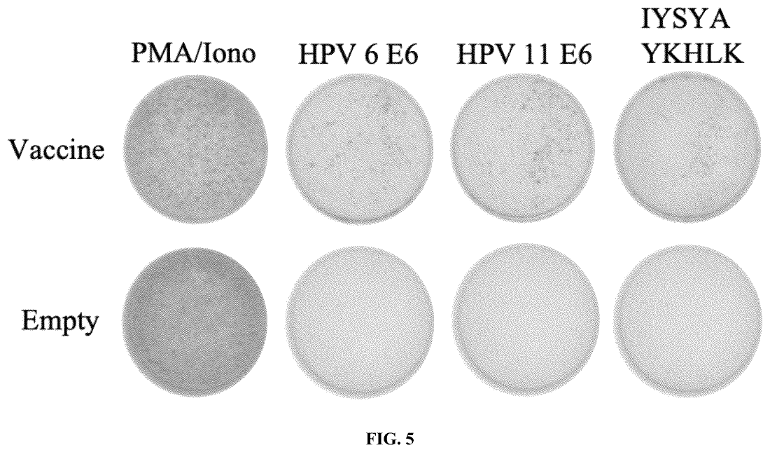

This in vitro stimulation resulted in IFNg production by T lymphocytes exposed to dendritic cells transduced with AdV-HPV6/11 but not an empty vector in all three participant samples. See FIG. 3. The adenoviral construct was able to elicit T cell responses in all 3 participants and therefore was used as the vaccine backbone for AdV-HPV6/11.

### Example 3: Induction by AdV-HPV6/11 of CD8-Dependent Immune Responses Sufficient to Inhibit HPV E6 Antigen-Positive Tumor Growth

This example demonstrates a method for inhibiting tumor growth of carcinomas engineered to express HPV6 E6 by inducing CD8-dependent immune responses.

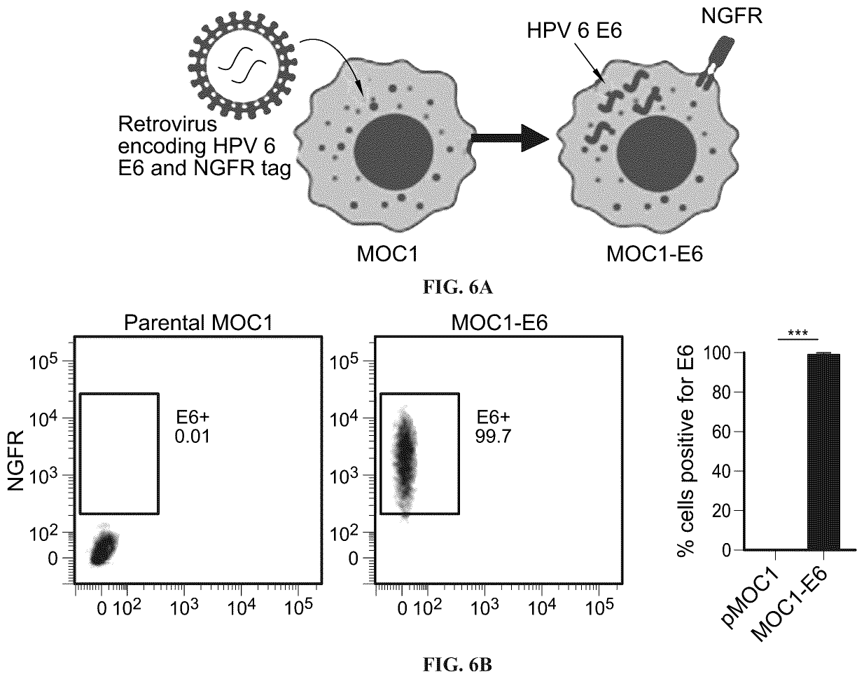

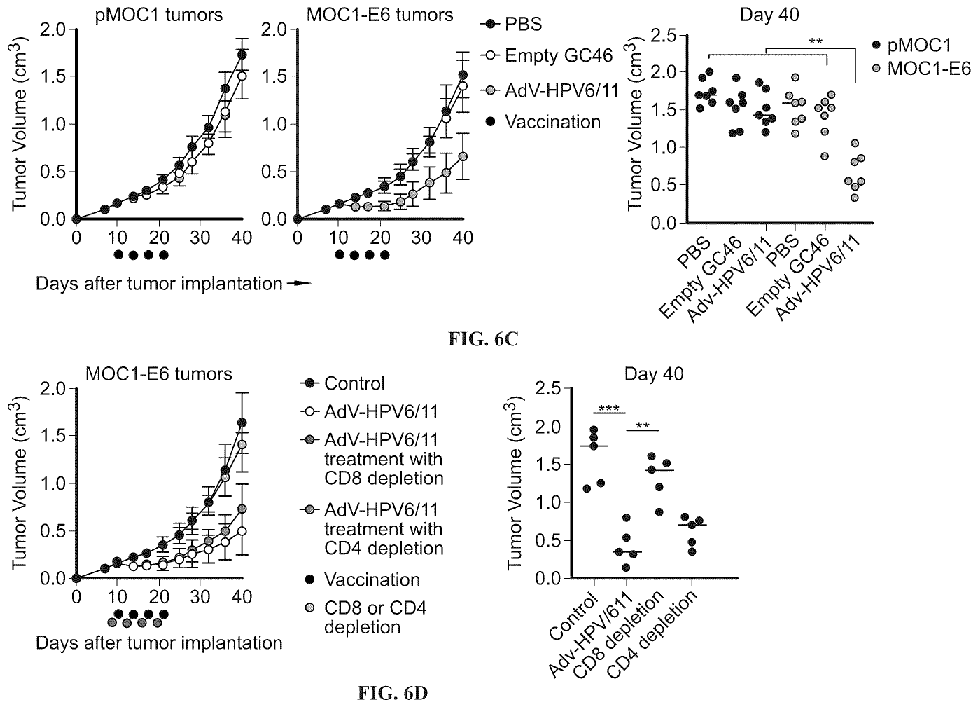

Given the lack of reproducible in vivo murine models of low-risk HPV-driven neoplastic diseases, a transplantable syngeneic mouse model of head and neck cancer (mouse oral cancer 1; MOC1) was engineered to express HPV6 E6 (FIG. 6A) with high purity (FIG. 6B).

Therapeutic vaccination of C57BL/6 mice bearing established MOC1-E6 tumors resulted in tumor growth delay (TGD) (FIG. 6C). Therapeutic vaccination with AdV-HPV6/11 had no effect on mice bearing parental MOC1 tumors lacking HPV6 E6. This experiment was repeated in the presence or absence of CD8 or CD4 depleting antibodies to establish which cell type was responsible for the observed TGD. Systemic depletion of CD8 but not CD4 T lymphocytes with monoclonal antibodies abrogated the TGD observed with AdV-HPV6/11 treatment of MOC1-E6 tumors (FIG. 6D).

These data established that therapeutic vaccination of mice bearing established E6 antigen-positive tumors with AdV-HPV6/11 can induce CD8-dependent immune responses sufficient to inhibit tumor growth.

### Example 4: Induction of Tumor Infiltration of Effector T Lymphocytes Specific for HPV E6 by AdV-HPV6/11

This example demonstrates a method for inducing tumor infiltration of effector T lymphocytes specific for HPV E6

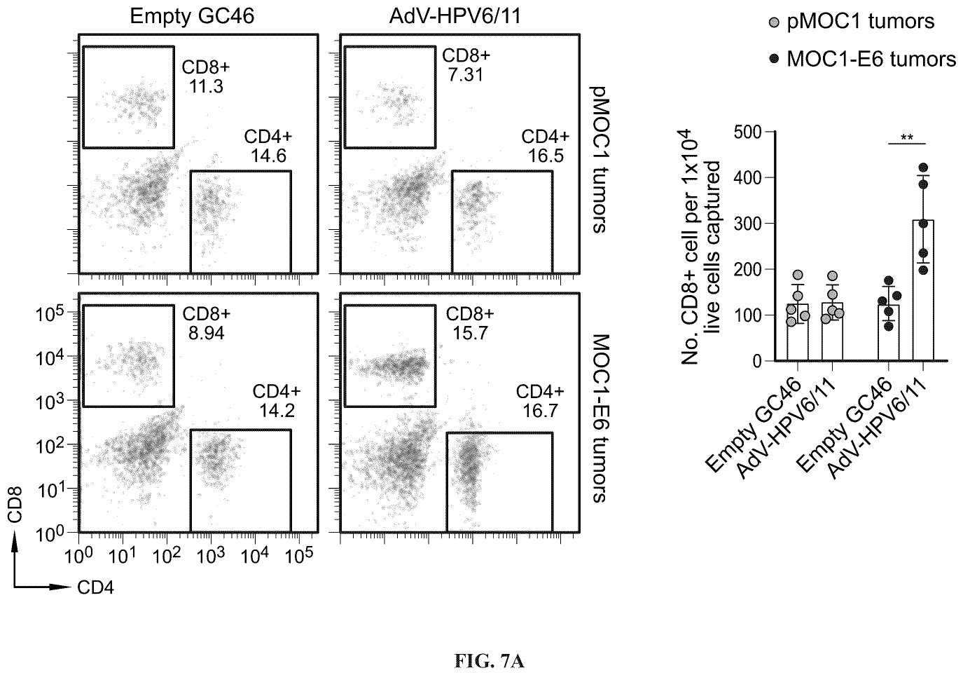

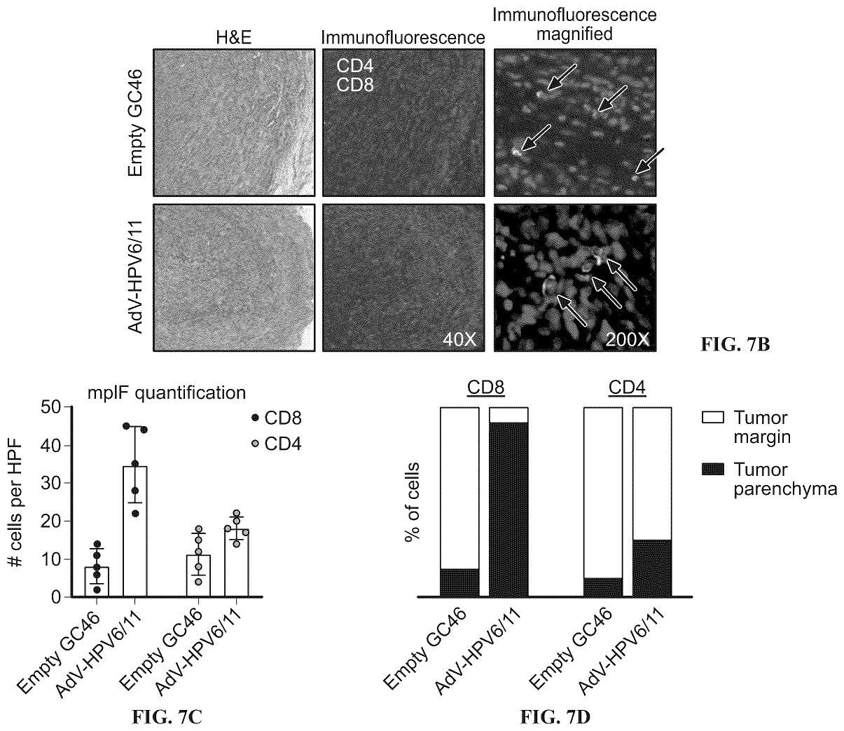

Flow cytometry of tumors harvested from parental MOC1 or MOC1-E6 tumors treated with AdV-HPV6/11 or empty GC46 was used to determine changes in T-lymphocyte infiltration. Significantly greater numbers of CD8+ T lymphocytes were present in MOC1-E6 tumors treated with AdV-HPV6/11, but not in parental MOC1 tumors treated with AdV-HPV6/11 or MOC1-E6 tumors treated with empty GC46 (FIG. 7A). Immunofluorescence was next used to determine the spatial localization of these T lymphocytes. Whereas analysis of MOC1-E6 tumors treated with empty GC46 revealed CD8+ T lymphocytes localized to the periphery of the tumor, MOC1-E6 tumors treated with AdV-HPV6/11 demonstrated increased numbers of CD8+ T lymphocytes that infiltrated throughout the tumor parenchyma (FIGS. 7B-D). AdV-HPV6/11 treatment did not significantly increase CD4+ T lymphocytes.

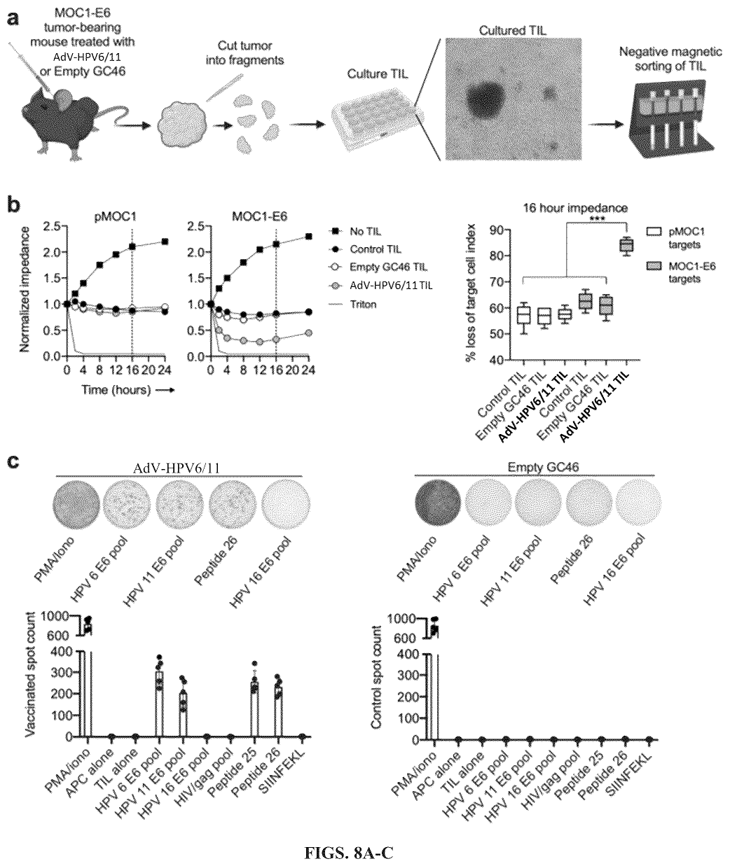

To experimentally determine if these T lymphocytes measured by flow cytometry and immunofluorescence within MOC1-E6 tumors represent E6-specific T lymphocytes, tumor-infiltrating lymphocyte (TIL) were cultured from tumors from mice treated with AdV-HPV6/11 or empty GC46 and tested for their ability to kill MOC1 cells expressing or lacking HPV6 E6 (FIG. 8A). TIL cultured from mice bearing MOC1-E6 tumors treated with PBS alone (control) or empty GC46 killed MOC1 cells expressing or lacking HPV6 E6 to a similar degree (FIG. 8B), suggesting the presence of TIL specific for endogenous MOC1 antigens. However, TIL cultured from MOC1-E6 tumors treated with AdV-HPV6/11 killed MOC1 cells expressing HPV6 E6 to a significantly greater degree than MOC1 cells lacking E6, suggesting the presence of E6-specific TIL. To validate the presence of E6-specific TIL, coculture ELISpot experiments were performed. TIL cultured from MOC1-E6 tumors treated with AdV-HPV6/11 but not empty GC46 demonstrated responses to 15-mer overlapping peptides corresponding to HPV6 E6 and HPV11 E6 as well as the peptides 25 and 26 (FIG. 8C), similar to the in vivo vaccination studies.

These data demonstrated that AdV-HPV6/11 can induce HPV-specific T lymphocytes that can traffic into E6 antigen-expressing neoplasms and induce effector responses sufficient to result in TGD.

### Example 5: AdV-HPV6/11 In-Human Phase I/II Study

This example describes an in-human phase I/II study of AdV-HPV6/11 for treating patients with RRP, specifically a phase I/II, 3+3 dose escalation clinical trial evaluating AdV-HPV6/11 at two dose levels (1×10¹¹ and 5×10¹¹ particle units (PU)) administered as adjuvant therapy after standard-of-care debulking surgery.

The protocol enrolled subjects with an RRP disease burden that required repeated surgical procedures for management. Participants with a pathologically confirmed diagnosis of papilloma and a clinical diagnosis of RRP were screened for the study's protocol. Participants who appeared to be eligible for treatment were examined via flexible nasopharyngolaryngoscopy and/or tracheoscopy in the outpatient clinic by the Otolaryngology Service. Participants were required to have laryngotracheal disease accessible for endoscopic surgical cleanout to be eligible, and participants with concurrent pulmonary RRP in addition to laryngotracheal disease were permitted. Participants that met the eligibility criteria were enrolled into the study.

In the phase I portion, the study enrolled up to 3-6 subjects at each dose level, and 12 subjects were treated at the maximum tolerated dose. In the phase II portion of the study, up to 40 participants (including participants treated at the recommended phase II adjuvant dose in the phase I portion of the study) may be treated in order to determine the percentage of participants who have an increase in their surgery-free interval following adjuvant treatment with AdV-HPV6/11.

After enrollment for Phase I, enrollment for the phase II portion began with participant enrollment. Participants underwent endoscopic operative removal of all visible papillomatous disease using standard techniques by the study team (e.g., removal of papilloma under general anesthesia and laser ablation of papilloma under local anesthesia). They received their first peripheral subcutaneous (SQ) injection with AdV-HPV6/11 after surgery. Participants may be observed as inpatients overnight (18-23 hours) for safety. Two weeks later (Day 15), participants underwent a clinical safety check and airway evaluation by the Otolaryngology Service, and outpatient administration of a second peripheral SQ injection of AdV-HPV6/11.

Around six weeks after receiving their first peripheral SQ injection with AdV-HPV6/11 (Day 43), participants underwent a clinical safety check and airway evaluation by the Otolaryngology Service. If papillomatous disease had recurred, the participant underwent a second endoscopic operative removal of all visible papillomatous disease using standard techniques. A third peripheral SQ dose of AdV-HPV6/11 was given after surgery. If participants had no recurrence of papillomatous disease at this 6-week timepoint, a third peripheral SQ injection of AdV-HPV6/11 was given without surgery.

Around twelve weeks after receiving their first peripheral SQ injection with AdV-HPV6/11 (Day 85), participants underwent a clinical safety check and airway evaluation by the Otolaryngology Service. If papillomatous disease had recurred, the participant underwent a third endoscopic operative removal of all visible papillomatous disease using standard techniques. A fourth peripheral SQ injection of AdV-HPV6/11 was given after surgery. If participants had no recurrence of papillomatous disease at the 12-week timepoint, a fourth peripheral SQ dose of AdV-HPV6/11 was without surgery.

In participants with pulmonary RRP, a CT neck/chest was performed at baseline and at the 6-week follow-up following completion of treatment, and responses were determined according to Response Evaluation Criteria in Solid Tumors (RECIST) criteria.

AdV-HPV6/11 was administered up to 4 separate SQ injections in separate limbs. The minimum time between administrations of AdV-HPV6/11 in each individual participant was 11 days. In the phase I portion of the study, dose escalation of AdV-HPV6/11 (1×10¹⁰ and 5×10¹⁰ PU) occurred in a standard 3+3 format. In the phase II portion of the study, participants will be treated with the recommended phase II adjuvant dosing (RP2D) which was determined to be 5×10¹¹ PU. The volume for each injection in all dose levels of AdV-HPV6/11 will be 1.0 mL.

Vital signs were measured within 30 minutes before and 30 minutes following administration of AdV-HPV6/11. Participants were observed for 2 hours after the first administration of AdV-HPV6/11. If no adverse reactions are observed, the participants were be monitored for only 30 minutes after subsequent doses. Participants returned at 6 weeks, 12 weeks and 24 weeks following the last administration of AdV-HPV6/11 for safety check and to assess for papilloma recurrence.

In coordination with the participant's home Otolaryngologist, surgical debridement of recurrent papillomatous disease may be performed, if clinically indicated. These visits, along with telephone contact every three months for two years after completion of these follow-up visits, will allow determination of secondary endpoints related to disease recurrence.

Where a participant's condition precluded safe performance of any protocol-driven biopsy, apheresis or other research procedure, the procedure may have been delayed for an additional two weeks or canceled at the discretion of the investigator. If such scenario occurred, it was not considered a protocol deviation.

An optional leukapheresis was performed at the first 6-week follow-up following administration of four doses of AdV-HPV6/11. This allowed investigators to obtain additional peripheral leukocytes for study from participants that have received AdV-HPV6/11 treatment. If participants were willing to undergo this optional leukapheresis, they were enrolled on protocol 16C0061 for banking of biospecimens. During operative interventions, samples for research were obtained from those participants who enroll on protocol 16C0061 for banking of biospecimens.

### Phase I Results

Patients. Fifteen total patients (10 male, 5 female) with histologically confirmed papilloma (FIG. 13) and the clinical diagnosis of RRP that required three or more clinically indicated procedures in the 12 months prior to receiving study therapy were treated with adjuvant AdV-HPV6/11 in this phase I study. Three patients were treated at dose level 1 (1×10¹¹ PU [DL1], patients 1-3) and 12 patients were treated at dose-level 2 (5×10¹¹ PU [DL2], patients 4-12). Each patient received 4 subcutaneous injections, 1 in each limb, over 12 weeks. The first injection was administered immediately following a surgical papilloma cleanout procedure and up to two papilloma removal procedures were performed as needed to maintain minimal residual disease during the 12-week vaccination period. The median age was 51 years (range 30-73 years). Two patients had juvenile-onset RRP with diagnoses prior to 12 years of age and 13 patients had adult-onset RRP. The median number of years since RRP diagnoses was 7 (range 1-43 years), and the median number of lifetime total clinically indicated procedures was 32 (range 9-300+). Patients had an average of 6.2 surgeries (range: 3-10) in the last 12 months before enrolling in the trial. Patients were diagnosed with RRP for an average of 15 years prior to enrollment. All patients had received at least one line of prior adjuvant therapy. Six patients had laryngeal without tracheal disease and 9 patients had both laryngeal and tracheal disease. Four patients had HPV11-related disease and 11 patients had HPV6-related disease. No association was observed between anatomic location of disease and HPV serotype (P=0.56, chi-square and Fisher's exact test).

Adverse Events. Repeated administrations of AdV-HPV6/11 were well-tolerated with no dose-limiting toxicities and no treatment-related adverse events (TRAEs) greater than Grade 2 (Table 7, below). All patients received four administrations of AdV-HPV6/11 at the intended dose level. TRAEs were all mild and reduced in frequency over the treatment interval. The most common TRAE was injection site reaction (self-limited local erythema, edema and/or tenderness), which occurred in all of the patients. Injection site reactions frequently occurred following any of the four individual subcutaneous injections and lasted up to one week. Most other TRAEs occurring in more than one subject were similar to seasonal vaccines and the most common were grade 1 fatigue, fever, and chills (Table 8, below). These symptoms were typically reduced or absent following subsequent injections. Two patients experienced grade 2 fatigue, one lasting for 2 days and the other lasting for 5 days, each occurring after the first injection. Two patients experienced grade 2 myalgia of 1-2 days duration, each treated with other-the-counter analgesics. DL2 was determined to be the recommended phase II dose (RP2D).

TABLE 7
Treatment-related Adverse Events
Total Patients (N = 15)

| | Dose Level 1 | | Dose Level 2 | | All Subjects | |
|---|---|---|---|---|---|---|
| | 1 × 10¹¹ PU (N = 3) | | 5 × 10¹¹ PU (N = 12) | | (N = 15) | |
| | Subjects | Events | Subjects | Events | Subjects | Events |
| | (N, %) | (N) | (N, %) | (N) | (N, %) | (N) |
| Grade 1 | 3 (100%) | 7 | 12 (100%) | 105 | 15 (100%) | 112 |
| Grade 2 | 0 (0%) | 0 | 2 (16.7%) | 4 | 2 (13.3%) | 4 |
| Grades 3-5 | 0 (0%) | 0 | 0 (0%) | 0 | 0 (0%) | 0 |

TABLE 8
Treatment-related Adverse Events by Grade
Total Patients (N =1 5)

| | Grade 1 | | | Grade 2 | | |
|---|---|---|---|---|---|---|
| | Subjects | | Events | Subjects | | Events |
| | (N, %) | | (N) | (N, %) | | (N) |
| Chills | 10/15 | (66.7%) | 14 | 0 | (0%) | 0 |
| Diarrhea | 1/15 | (6.7%) | 1 | 0 | (0%) | 0 |
| Shortness of | 1/15 | (6.7%) | 1 | 0 | (0%) | 0 |
| breath (Dyspnea) | | | | | | |
| Excessive | 2/15 | (13.3%) | 2 | 0 | (0%) | 0 |
| sweating | | | | | | |
| (Hyperhidrosis) | | | | | | |
| Fatigue | 9/15 | (60.0%) | 20 | 2/15 | (13.3%) | 2 |
| Fever | 9/15 | (60.0%) | 17 | 0 | (0%) | 0 |
| Injection site | 15/15 | (100%) | 46 | 0 | (0%) | 0 |
| reaction | | | | | | |
| Muscle | 2/15 | (13.3%) | 2 | 2/15 | (13.3%) | 2 |
| aches (Myalgia) | | | | | | |
| Nausea | 4/15 | (26.7%) | 6 | 0 | (0%) | 0 |
| Skin | 1/15 | (6.7%) | 1 | 0 | (0%) | 0 |
| itching (Pruritus) | | | | | | |
| Vomiting | 2/15 | (13.3%) | 2 | 0 | (0%) | 0 |

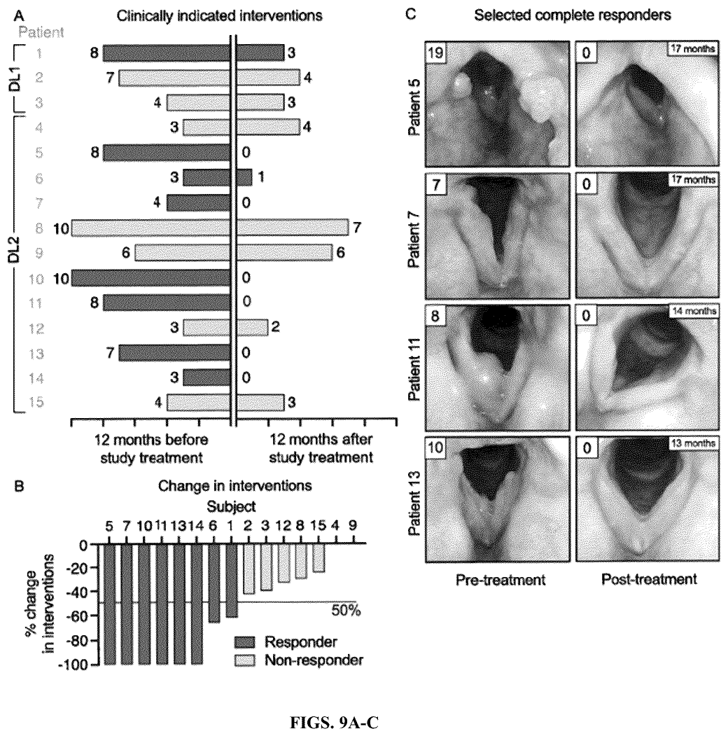

Phase II—Clinical data. To assess clinical response to AdV-HPV6/11, the number of clinically indicated interventions in the 12 months after the study treatment was compared to the number of clinically indicated interventions is the 12 months before the study treatment (FIGS. 9A and B). Among the 15 patients, the median number of interventions in the 12 months before the study was 6. All patients treated with AdV-HPV6/11 at dose level 1 experienced a decrease in the number of interventions in the 12 months following completion of study treatment, and 1/3 (33%) experienced a partial response. The clinical data further show AdV-HPV6/11 treatment significantly reduced the need for surgeries for severe, aggressive RRP patients treated at Dose Level 2. The number of RRP surgeries in the patients (N=12) in Dose Level 2 reduced from a median of 6.5 surgeries (range: 3-10) in the 12-months pre-treatment to 0.5 surgeries (range: 0-6) in 12-months post AdV-HPV6/11 treatment completion. At Dose Level 2, 50% (6 out of 12) patients had a Complete Response, which is defined as no surgeries needed during the 12-month period following AdV-HPV6/11 treatment completion (Table 9, below). Complete responses were durable and ongoing for the 6 CRs, with no interventions required for a median of 15.5 months (range 14-18 months) at the time of data cutoff. Patients in Dose Level 2 had a 58% (7 out of 12) Overall Response Rate, defined as greater than or equal to 50% reduction in the surgeries in 12-months post AdV-HPV6/11 treatment completion compared to 12-months pre-treatment. 83% (10 out of 12) of patients treated at Dose Level 2 had reduced surgeries post AdV-HPV6/11 treatment.

Defining partial response (PR) as a 50% or greater reduction in the need for procedures following completion of study treatment, a PR was observed 1 of 12 patients treated at DL2, giving an overall DL2 response rate (CR+PR) of 58% (7 of 12 patients). One PR was observed at DL1, giving an overall study response rate of 53% (8 responders, 7 non-responders). Considering protocol-indicated surgical cleanout procedures required to maintain minimal residual disease throughout the study period, non-responders consistently required 2 of 2 on-study optional procedures during the study period, whereas all responders required 0 or 1 on-study procedures. No clear differences were observed between responders and non-responders considering the number of lifetime interventions before the trial (P=0.10, Mann Whitney two-tailed test), number of interventions in the 1 year before receiving study treatment (P=0.42), age at treatment (P=0.28), gender (P=0.99, Chi-square with Fisher's exact test), HPV type (P=0.28), the presence of tracheal disease (P=0.12) or individual HLA allele type. Baseline disease burden, measured by calculating the anatomic Derkay score, also did not predict response (P=0.20).

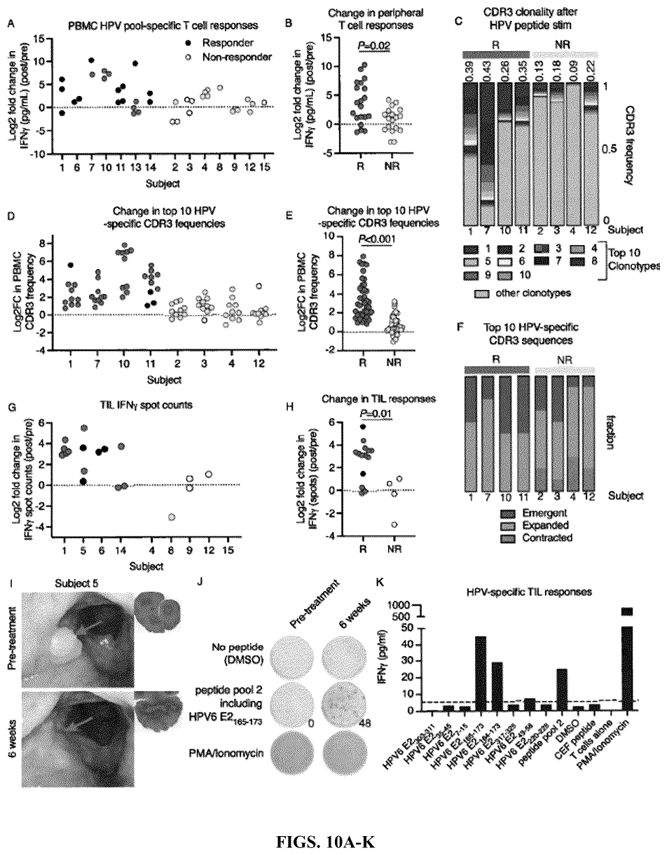

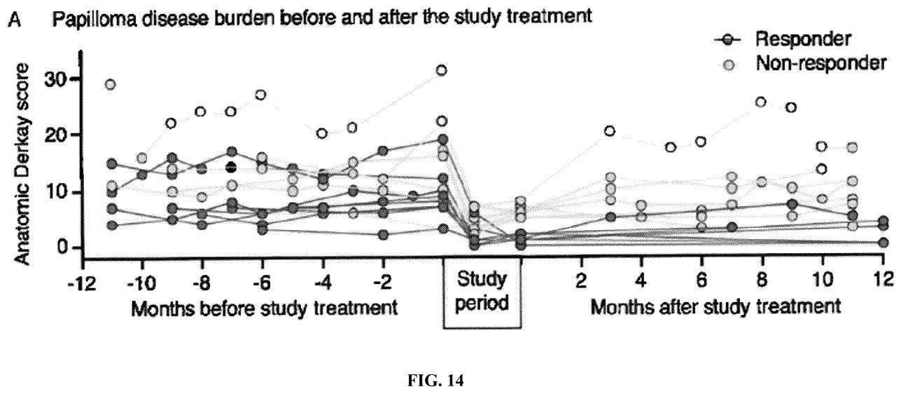

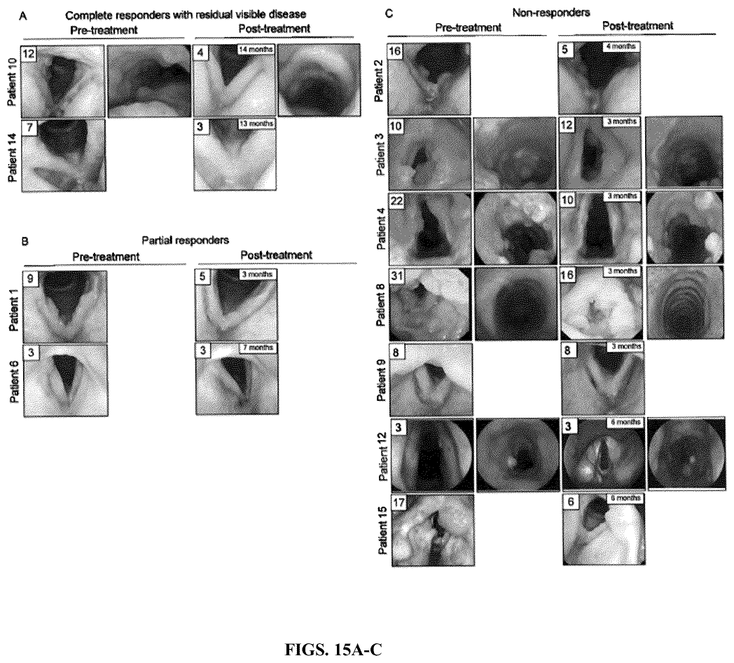

Further, AdV-HPV6/11 treatment showed significant improvement in anatomical Derkay scores, a tool used for research purposes to quantify RRP severity based on involvement of laryngeal structures, and voice quality, evaluated using the validated Vocal Handicap Index-10 (VHI-10). Longitudinal tracking of papilloma disease burden as measured by the anatomic Derkay score was assessed by scoring available representative endoscopic images from the 12 months before and after the study treatment for each patient (FIG. 14; see also FIG. 10I at 6 weeks). Complete visual resolution of disease was observed in 4 of 6 CRs (67%, FIG. 9C, representative endoscopic images of remaining patients in FIGS. 15A-C). Several small foci of true vocal fold papilloma were evident in one CR patient (patient 14), and several small foci of vocal fold and tracheal papilloma were evident in another patient (patient 10) that did not cause symptoms necessitating intervention. Papilloma disease burden was relatively consistent prior to initiation of AdV-HPV6/11 treatment for each patient. After the trial, low disease burden or no detectable papilloma was observed in the responders and return of disease burden similar to pre-trial levels was observed in the non-responders.

In addition to establishing the safety and tolerability of adjuvant AdV-HPV6/11 in adults with RRP, these data indicate that AdV-HPV6/11 is a robust treatment option for RRP since treatment with AdV-HPV6/11 in this study durably reduced papilloma disease recurrence and the need for clinically indicated procedures in greater than 50% of patients treated at the RP2D.

TABLE 9
Clinical Efficacy Summary
Total Patients (N = 15)

| | Dose | Dose |
|---|---|---|
| | Level 1 | Level 2 |
| | (N = 3) | (N = 12) |
| Complete Response (CR) | 0% | 50% |
| No surgeries needed during 12-months post- | (0/3) | (6/12) |
| treatment | | |
| Overall Response Rate (ORR) | 33% | 58% |
| ≥50% reduction in surgeries during 12-months | (1/3) | (7/12) |
| post-treatment compared to 12-months pre-treatment | | |
| Decrease in Rate of Surgery | 100% | 83% |
| 12-months post-treatment compared to 12-months | (3/3) | (10/12) |
| pre-treatment | | |

AdV-HPV6/11 induces peripheral blood HPV-specific T cell responses. To assess the mechanisms underlying clinical response to AdV-HPV6/11, peripheral blood T cells before and after treatment were assayed for HPV-specific responses following stimulation with pools of HPV 6 and 11 peptides encoded in AdV-HPV6/11 (FIG. 10A). Eleven of 14 patients (76%) with evaluable PBMC demonstrated measurable peripheral blood HPV-specific T cell responses prior to treatment. Thirteen of 14 patients (93%) demonstrated expansion of peripheral HPV-specific T cell responses after treatment. Overall, patients that developed clinical responses following treatment with AdV-HPV6/11 displayed a greater magnitude of expansion of peripheral HPV-specific T cell responses compared to non-responders (FIG. 10B, P=0.02, Mann-Whitney two-tailed test). Notably, while 7 of 7 (100%) responders with evaluable clinical material demonstrated expansion of HPV-specific T cell responses to more than one HPV peptide pool, suggesting the presence of a polyclonal HPV-specific T cell response, only three of 7 (43%) non-responders developed expanded HPV-specific T cell responses to more than one HPV peptide pool. Baseline and treatment-enhanced HPV pool-specific responses in the peripheral blood consistently were specific to the HPV type (6 or 11) detected in the papilloma for each patient.

Taken together, the foregoing data indicates that AdV-HPV6/11 treatment consistently results in increased HPV-specific peripheral blood T cell responses.

AdV-HPV6/11 induces papilloma infiltrating T cell HPV-specific responses. T cells cultured from available papilloma biopsies (papilloma infiltrating lymphocytes, PIL) were assayed for HPV peptide pool-specific responses in overnight co-culture assays. Post-treatment papilloma biopsies were not available from four responders that did not develop papilloma recurrence after initiation of AdV-HPV6/11 treatment. In all responders with paired samples, increased HPV-specific T cell responses were detected in post-treatment PIL compared to pre-treatment PIL and polyclonal HPV-specific PIL responses were observed in three of four patients (FIG. 10G).

In five paired samples from non-responders, low magnitude increases in HPV-specific PIL responses were observed in two patients, a reduction in response was observed in one patient, and no responses were detected in two patients. Overall, greater magnitude of a higher number of HPV-specific responses were observed in PIL from responders compared to non-responders (FIG. 10H, P=0.01, Mann-Whitney two-tailed test). A strong post-treatment PIL response to HPV peptide pool 2 in patient 5 that was undetectable in pre-treatment PIL biopsied from the same anatomic location was studied further (FIGS. 101 and 10J). Co-culture of patient 5 post-treatment PIL with antigen presenting cells loaded with individual minimal peptides included in pool 2 identified responses to the HPV6 E2₁₆₅₋₁₇₃ 9-mer peptide (FIG. 10K).

Together, these data indicate that peripheral administration of AdV-HPV6/11 induces HPV-specific T cell responses that can traffic into the papilloma microenvironment.

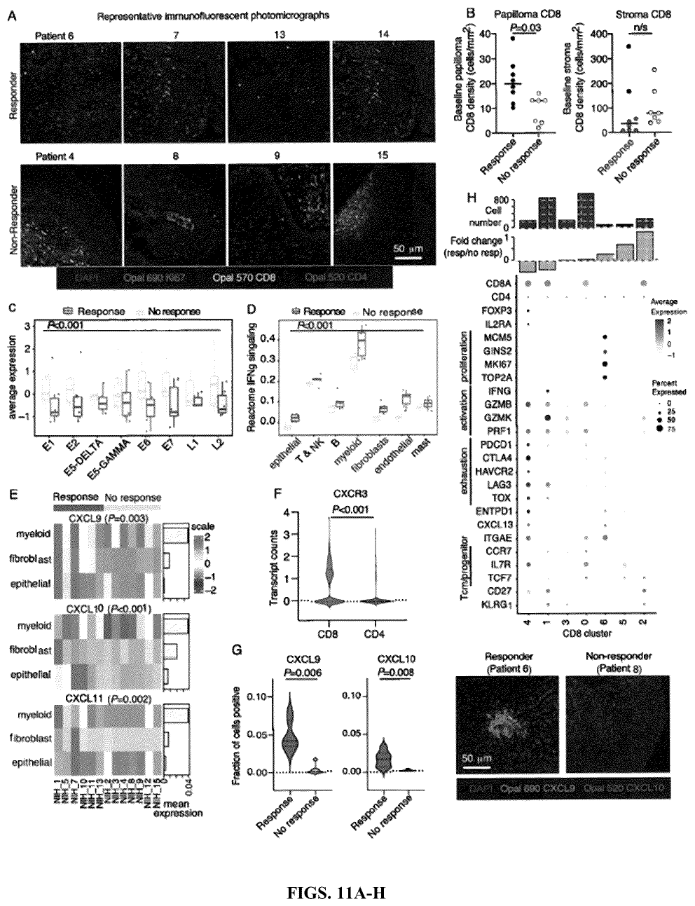

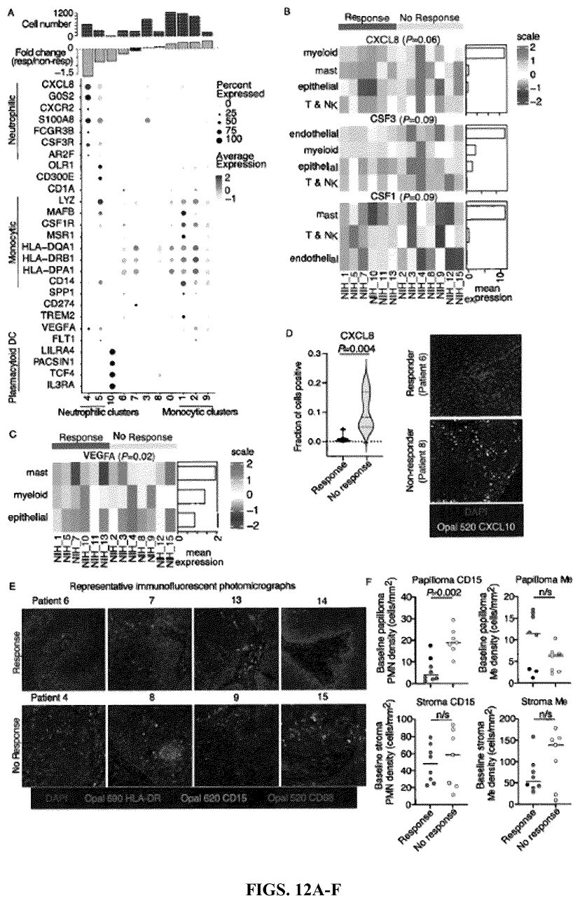

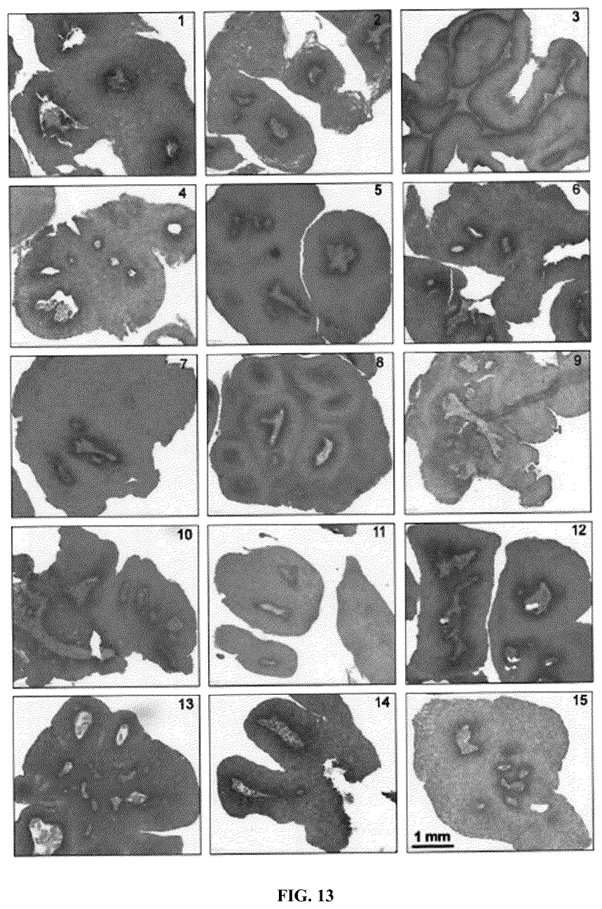

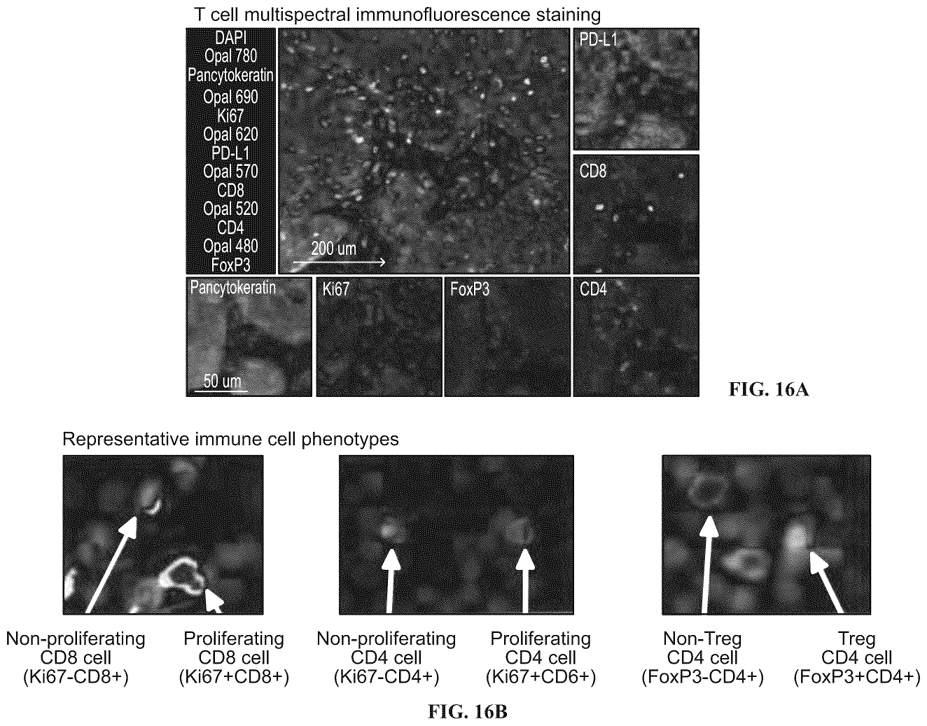

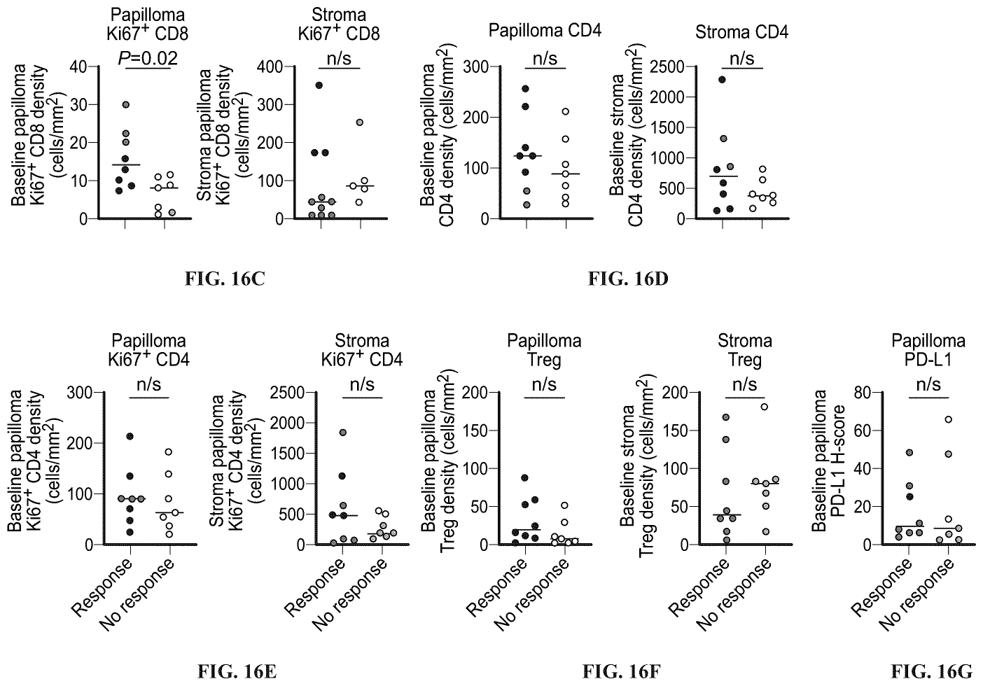

HPV gene expression, papilloma inflammation, and response to AdV-HPV6/11. Immunofluorescence was performed to determine T cell quantity and localization within pre-treatment papilloma tissues collected from a subset patients with low HPV-specific PIL reactivity and lack of clinical response. This analysis revealed increased density of total and proliferating CD8 T cells into the papilloma parenchyma of responders compared to non-responders, but no difference in CD8 T cell density in the stroma (FIGS. 11A and 111B). Similarly, no clear differences were observed in total or proliferating CD4 T cells in the papilloma parenchyma or stroma, regulatory CD4 T cells in the or papilloma parenchyma or stoma, or papilloma PD-L1 expression between responders and non-responders (FIGS. 16D-G). These data point to a selective defect in the ability of CD8 T cells to infiltrate the papilloma parenchyma in patients that do not develop a clinical response to AdV-HPV6/11.

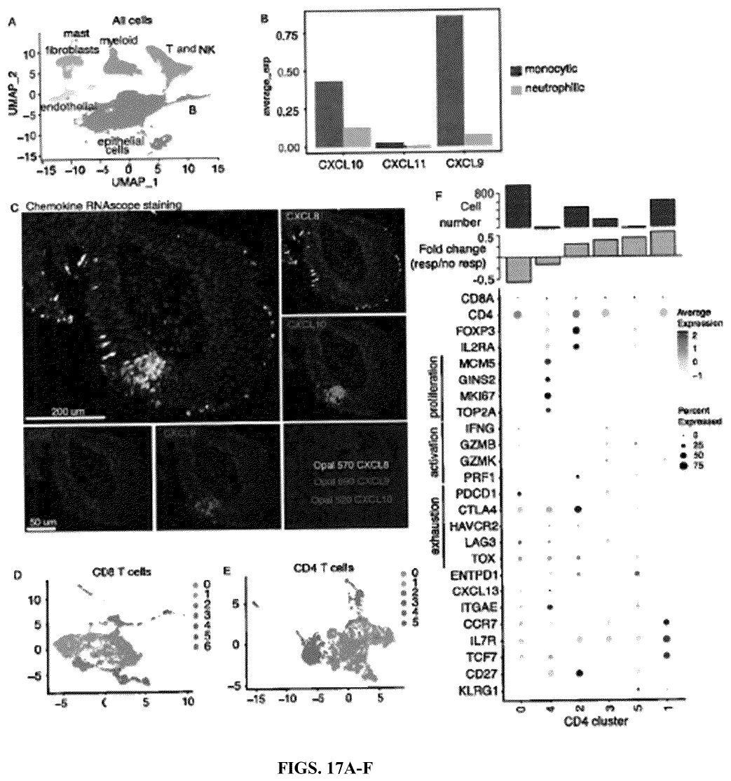

Further, to explore possible underlying mechanisms regulating T cell trafficking in these samples, single cell transcriptomes from fresh pre-treatment papilloma biopsies in 13 of 15 patients, including 6 responders and 7 non-responders was studied (FIG. 17A). Of 75,934 individually sequenced cells, 43,519 were identified as epithelial papilloma cells. Considering expression of all detectable HPV genes, cell-normalized HPV gene expression was significantly reduced in responders compared to non-responders (FIG. 11C) demonstrating an association between magnitude of HPV gene expression and response to AdV-HPV6/11. Gene set enrichment analysis aimed at identifying further differences based upon response identified greater IFNγ signaling in epithelial, stromal, and immune cells in responders compared to non-responders (FIG. 11D).

Next, the expression of the IFN-inducible chemokines CXCL9, CXCL10 and CXCL11 was explored given their role in regulating T cell trafficking. These chemokines, expressed primarily in papilloma-infiltrating myeloid cells but also in fibroblasts and epithelial papilloma cells, were expressed to a greater degree in responders compared to non-responders (FIG. 11E). Within the myeloid compartment, CXCL9/10/11 was predominantly expressed in monocytic and not neutrophilic myeloid cells (FIG. 17B). Greater expression of CXCR3, the cognate receptor for CXCL9/10/11, was observed on CD8 T cells compared to CD4 T cells (FIG. 11F), possibly accounting for the enhanced papilloma parenchyma infiltration of CD8 T cells but not CD4 T cells in responders with high expression of these T cell chemokines. Detection of greater CXCL9 and CXCL10 expression in responders compared to non-responders using single-cell transcriptomics was validated histologically using RNAscope in situ hybridization (FIG. 11G, FIG. 17C). Since papilloma and immune cells that express CXCL9/10 form clusters, papilloma cell HPV gene expression appears to inversely correlate with IFN responses and production of T cell chemokines within the papilloma microenvironment.

In addition, transcriptional analysis was performed on T cells infiltrating the papillomas prior to treatment with AdV-HPV6/11 to assess whether the transcriptional profile of these T cells associated with clinical response. Single cell transcriptional analysis of 12,918 papilloma infiltrating T cells revealed different T cell cluster identities and transcriptional states. Relative enrichment of CD8 (cluster 2) and CD4 (cluster 1) cell clusters expressing central memory and progenitor T cell markers TCF7, IL7R and CCR7 was observed in responders (FIG. 11H; FIGS. 17D-F). Enrichment of clusters (CD8 clusters 1,4; CD4 clusters 0,4) expressing activation (IFNG, GZM, PRF1) and exhaustion markers (PDCD1, CTLA4, HAVCR2, LAG3, TOX) was observed in non-responders with higher HPV gene expression. Notably, expression of markers previously linked to tumor antigen-specificity of CD8 T cells such as ENTPD1 encoding for CD39, ITGAE encoding CD103 and CXCL13¹⁷⁻¹⁹ were expressed to the greatest degree in T cells enriched at baseline in non-responders (cluster 4), indicating that T cells infiltrating these papillomas may become suppressed and dysfunctional in the face of chronic antigen exposure.

Together, these data associate reduced HPV gene expression, greater IFNγ response signatures and greater CXCL9/10/11 expression with a papilloma microenvironment that promotes infiltration of T cells at baseline and clinical response to AdV-HPV6/11 treatment. Conversely, the papilloma microenvironment observed in non-responders with greater HPV antigen burden, lower IFNγ responses and lower CXCL9/10/11 expression appears to poorly recruit T cells and promote lymphocyte exhaustion and dysfunction.

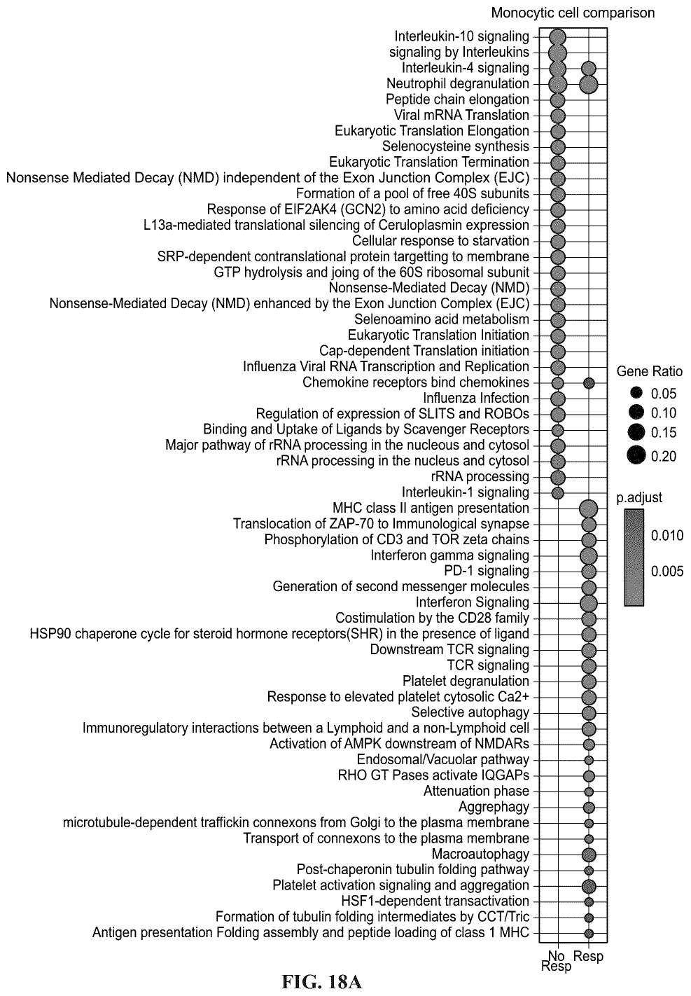

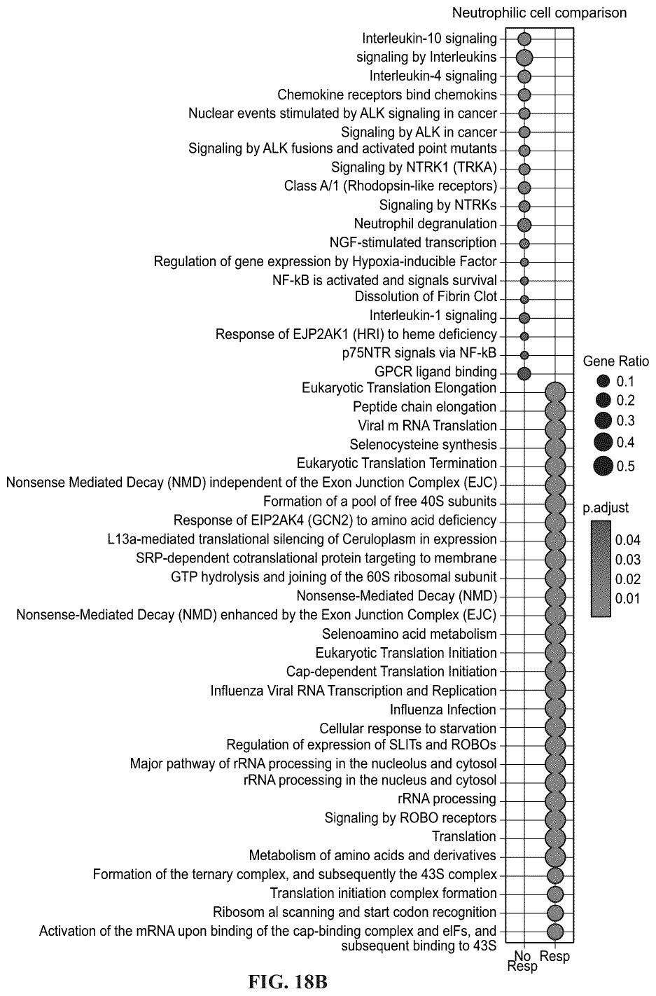

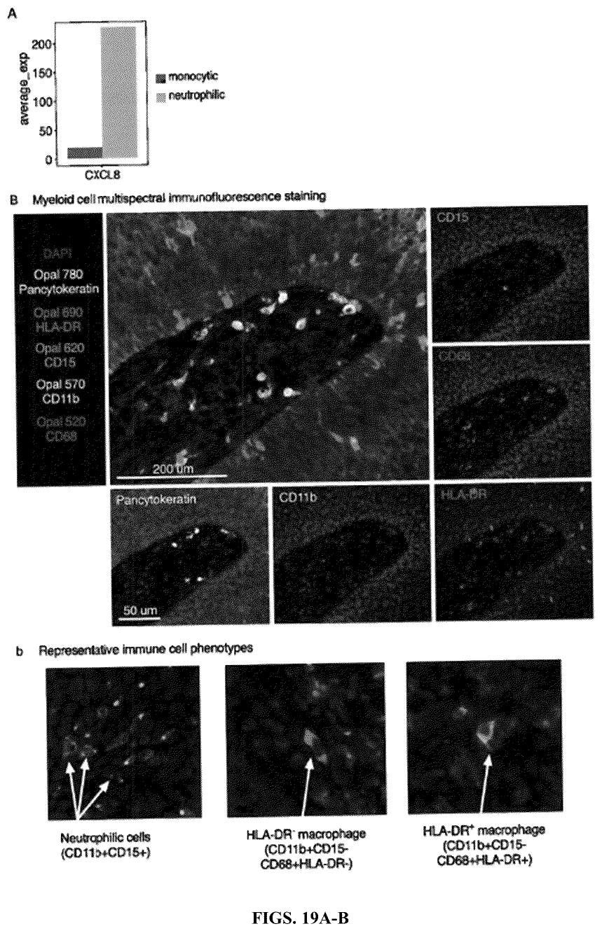

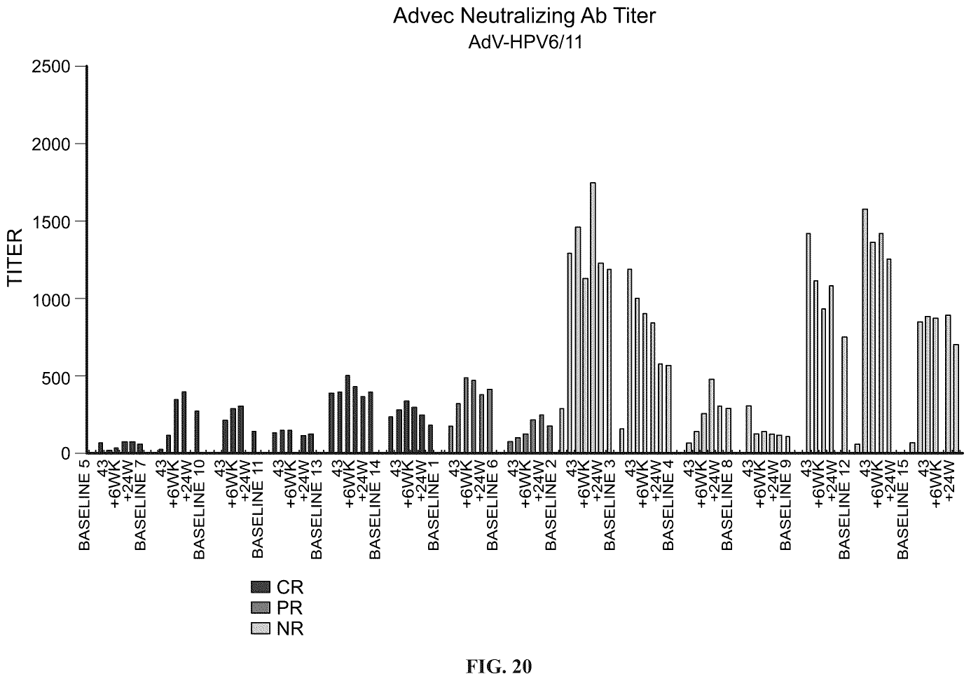

In light of the observations relating to myeloid-derived T cell chemokines, T cell infiltration, and clinical benefit, myeloid cells were studied to assess whether other myeloid parameters in pre-treatment papillomas also associated with clinical response to AdV-HPV6/11. Among 7,629 individually sequenced myeloid cells, multiple cluster identities were identified (FIG. 12A), including monocytic clusters (1, 2, 9) that were enriched in patients that developed a clinical response to AdV-HPV6/11 and neutrophilic clusters (4, 5) that were enriched in non-responders. Notably, myeloid cluster 5, enriched in non-responders, expresses neutrophilic and monocytic lineage markers indicative of an undifferentiated state and a myeloid derived suppressor cell (MDSC) phenotype. Expression of OLR1 (Lox1) and CD300e, previously linked to immunosuppressive capacity, is greatest in cluster 5 further supporting that these cells may represent neutrophilic-MDSC. To further compare transcriptional differences in papilloma infiltrating myeloid cells between responders and non-responders, gene set enrichment analysis (GSEA) was conducted (FIGS. 18A and 18B). Both monocytic and neutrophilic cells from non-responders were enriched for terms related to Th2 cytokine signaling (IL-10 and IL-4), IL-1 signaling, and neutrophil degranulation. Consistent with T cell-permissive microenvironment, monocytic cells from responders were enriched for terms related to interferon and TCR signaling and antigen processing and presentation.

Further, it was assessed whether the expression of myeloid chemokines associated with myeloid infiltration and clinical response. The chemokines CXCLB (I1-8) and CSF3 (G-CSF) regulate myeloid chemotaxis through expression of the CXCR2 and CSF3R receptors, respectively. A trend toward greater CXCL8 (P=0.06, two-way !NOVA), primarily expressed by neutrophilic myeloid cells (FIG. 19A), and CSF3 (P=0.09), primarily expressed by endothelial cells, associated with enrichment of neutrophils that express CXCR2 and CSF3R in non-responders (FIGS. 12A and 12B). The chemokine CSF1 (M-CSF) regulates myeloid chemotaxis through expression of CSF1R. A trend toward greater CSF1 (P=0.10), primarily expressed by mast cells, associated with enrichment of monocytic cells that express CSF1R in responders. VEGF, important for the rich vascularization observed in papillomas, was expressed to a significantly greater degree in the mast, myeloid and epithelial cells of non-responders compared to responders (FIG. 12C). Greater CXCL8 expression in non-responders compared to responders observed in the single cell sequencing dataset was validated histologically using RNAscope in situ hybridization (FIG. 12D, FIG. 17C). Immunofluorescence analysis of baseline papilloma samples revealed greater CD15 positive neutrophilic cell density in the papilloma parenchyma of non-responders compared to responders (FIGS. 12E and 12F, FIG. 19B). No clear difference was observed in monocytic cell (macrophage) density.

These data support a paradigm whereby papilloma cells govern clinical response to AdV-HPV6/11 through modulation of the chemokine and immune profile of the papilloma microenvironment (graphical abstract). Papillomas with low HPV gene expression form a T cell permissive microenvironment high in monocyte derived CXCL9/10/11 that associates with clinical response. Alternatively, papillomas with high HPV gene expression harbor exhausted T cells and form a microenvironment high in CXCL8 and neutrophils that express markers of immunosuppression and associate with no clinical response.

Neutralizing antibody titers. Neutralizing antibody assays were performed on serum samples collected from the phase 1 participants using the general, exemplary methodology disclosed in Johnson T R et al., “Genetic vaccine for respiratory syncytial virus provides protection without disease potentiation,” Mol Ther 22:196-205 (2014) and Limbach, K. et al., “New gorilla adenovirus vaccine vectors induce potent immune responses and protection in a mouse malaria model,” Malar J. 16:263 (2017). Briefly, serum samples were diluted in DMEM at 1:16 to 1:8192, incubated with GC46 vectors expressing the firefly luciferase gene for 1 h at room temperature, and then used to infect 5×10⁴ A549 cells in triplicate at a multiplicity of infection (MOI) of 2000 virus particle units (pu)/cell. Twenty-four hours post-infection, the cells were lysed and luciferase activity was measured using the ONE-Glo™ Luciferase Assay (Promega). Samples that resulted in >90% reduction in luciferase activity compared to the virus-only control were defined as positive for neutralizing antibodies. The endpoint titer was defined as the maximum dilution at which the serum sample displayed a 90% reduction in luciferase activity compared to the virus-only control.

The assay results depicted in FIG. 20 indicate that, pre-treatment (baseline), patients did not possess neutralizing antibodies for the GC46 vector. However, post-treatment with AdV-HPV6/11, some (but not all) patients exhibited neutralizing antibodies to the GC46 vector. Specifically, titers were undetectable or detectable at very low levels prior to study treatment, consistent with previous observations of low rates of seroprevalence of neutralizing antibodies against gorilla adenoviruses compared to human Ad5. Quantitatively low titers were detected after initiation of the study treatment in all responders and in 2 of 7 non-responders. Higher titers were observed after initiation of the study treatment in in 5 of 7 non-responders. Consistent increases in titers with repeat administration of AdV-HPV6/11 were not observed in any patient.

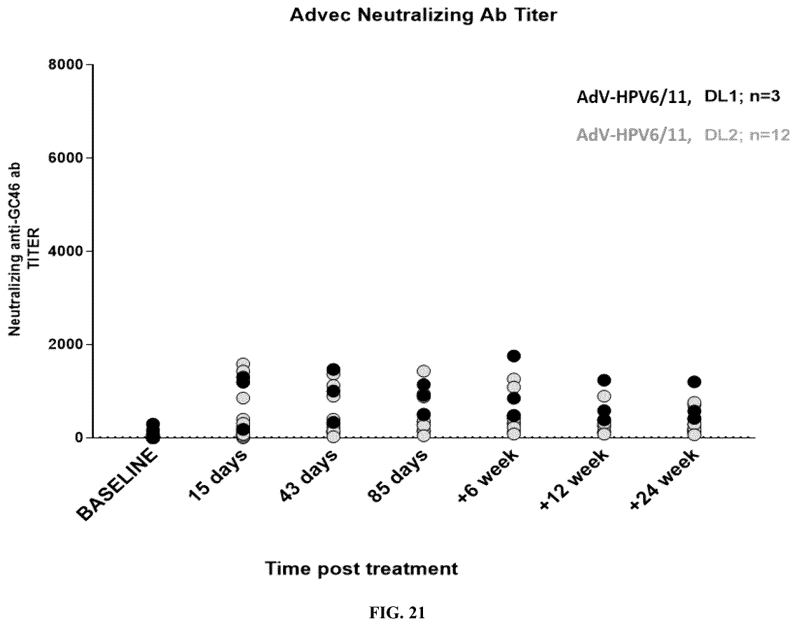

This demonstrates that the GC46 vector itself likely is not affecting the observed immune response but, rather, it is the HPV6/11 antigens that are triggering the immune response. Similarly, the assay results in FIG. 21 indicate that there is no clear correlation between the dosage level of the GC46 vector and detection of neutralizing antibodies over the course of AdV-HPV6/11 treatment.

Accordingly, this data suggests that the GC46 vector itself is not affecting the observed clinical response to AdV-HPV6/11 treatment and that the measured response is attributable to T cell generation in response to the novel HPV6/11 antigens encoded by AdV-HPV6/11. The lack of a significant neutralizing antibody response to gorilla adenovector over time with subsequent additional vaccinations highlights the ability to deliver repeated administrations of AdV-HPV6/11, a differentiating feature of the proprietary gorilla adenovirus technology comprising AdV-HPV6/11. The clinical significance of the observation of greater titers in 5 of 7 non-responders compared to the rest of the study patients, however, is unclear.

### Example 6: Additional Results from AdV-HPV6/11 In-Human Phase I/II Study

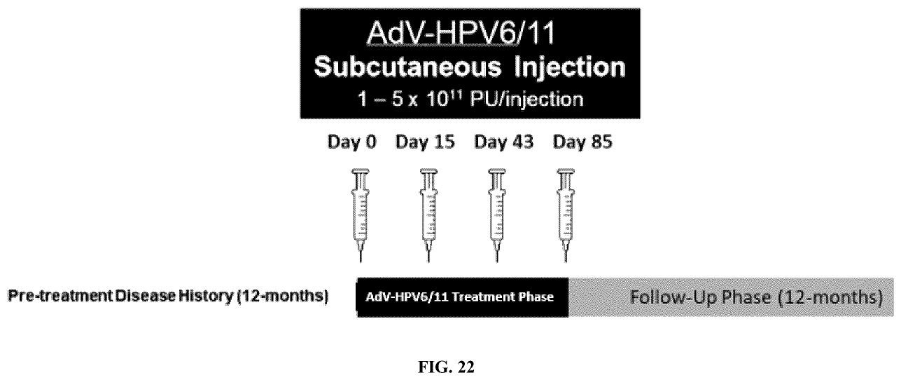

A phase I/II study was conducted following the protocols described in Example 5, with any deviations noted in this example (FIG. 22). The study investigated the immunogenicity, safety, and efficacy of AdV-HPV6/11, a recombinant adenoviral vector expressing HPV 6 and 11 antigens, in patients with recurrent respiratory papillomatosis (RRP).

The results demonstrated that AdV-HPV6/11 successfully elicited HPV 6/11-specific T cell immune responses in the treated patients. The vaccine was found to be safe and well-tolerated, with no significant adverse events reported. Notably, more than 50% of the patients who received the AdV-HPV6/11 vaccine did not require any surgical intervention for their RRP for at least 12 months following the treatment. The median duration of complete response has not yet been reached, with a median follow-up period of 20 months, indicating a potentially long-lasting effect of the vaccine.

The study included a specific number of participants, and the dose per injection was carefully determined. The demographic characteristics of the patients enrolled in the study are provided in Table 10 and Table 11. These tables summarize important information such as age, gender, and other relevant clinical data of the participants.

In conclusion, this phase 1/11 study demonstrates the promising immunogenicity, safety, and efficacy of the AdV-HPV6/11 vaccine in patients with RRP. The results suggest that this vaccine may provide a long-lasting effect in reducing the need for surgical interventions in RRP patients. Further studies with larger patient cohorts and longer follow-up periods may be necessary to confirm these findings and assess the long-term benefits of the AdV-HPV6/11 vaccine in managing RRP.

### Primary Endpoints

Safety: The study evaluated the safety profile of AdV-HPV6/11 in patients with recurrent respiratory papillomatosis (RRP). The safety assessment included monitoring adverse events, laboratory parameters, and vital signs throughout the study period.

Recommended Phase 2 Dose (RP2D): The study aimed to determine the optimal dose of AdV-HPV6/11 for future Phase 2 trials based on the safety and efficacy data collected during the Phase I/II study.

### Complete Response Rate

The complete response rate was defined as the percentage of patients who did not require any surgical intervention to control their RRP in the 12 months following the completion of the AdV-HPV6/11 treatment. This endpoint assesses the long-term efficacy of the vaccine in reducing the need for surgical procedures in RRP patients.

### Secondary Endpoints

HPV-specific Immune Responses: The study evaluated the ability of AdV-HPV6/11 to elicit HPV 6/11-specific immune responses in the treated patients. This assessment included measuring the presence and magnitude of HPV 6/11-specific antibodies and T cell responses following vaccination.

Extent of Papilloma Growth (Derkay Scores): The Derkay score is a clinical assessment tool used to quantify the extent and severity of papilloma growth in RRP patients. The study evaluated changes in the Derkay scores of the participants before and after treatment with AdV-HPV6/11 to assess the vaccine's impact on papilloma growth.

Vocal Function (Vocal Handicap Index: VHI-10): The VHI-10 is a patient-reported outcome measure that assesses the impact of voice disorders on a patient's quality of life. The study evaluated changes in the VHI-10 scores of the participants before and after treatment with AdV-HPV6/11 to determine the vaccine's effect on vocal function and voice-related quality of life.

QoL (Quality of Life): A QOL score is a measure used to quantify an individual's overall well-being and satisfaction with life, taking into account various aspects such as physical health, psychological state, social relationships, and environment.

These endpoints collectively provide a comprehensive assessment of the safety, efficacy, and immunogenicity of the AdV-HPV6/11 vaccine in patients with RRP. The results from this Phase I/II study will inform the design and implementation of future clinical trials to further evaluate the potential of AdV-HPV6/11 as a novel therapeutic approach for RRP.

TABLE 10
Number of Participants Enrolled and Injection Dose

| # Participants Enrolled | Dose Per Injection |
|---|---|
| N = 3 | DL1: 1 × 10¹¹ PU |
| N = 35 | DL2: 5 × 10¹¹ PU |

TABLE 11
Patient Characteristics.

| | Dose Level 1 | | Dose Level 2 | |
|---|---|---|---|---|
| | (N = 3) | | (N=35) | |
| Mean age (range), years | 56.3 | (39-67) | 49.3 | (20-88) |
| Male | 3 | (100%) | 20 | (57%) |
| Patient Characteristics | | | | |
| Age at Diagnosis (years) | 25-60 | | 1-68 | |
| Juvenile onset | 0 | (0%) | 12 | (34%) |
| Adult onset | 3 | (100%) | 23 | (66%) |
| Mean age at diagnosis | 38 | (25-60) | 29 | (1-68) |
| Mean Years since | 19 | (3-42) | 20 | (1-65) |
| diagnosis | | | | |
| Baseline Disease | | | | |

| | | |
|---|---|---|
| Surgeries in last 12 | Mean 6.3 | Mean 4.5 (range 3-10) |
| months | (range 4-8) | |
| Derkay Score | Mean 11.7 (9-16) | Mean 10.6 (range 3-31) |
| QOL score | Mean 27 (17-36) | Mean 23.5 (range 6-40) |

Outcomes: The AdV-HPV6/11 treatment demonstrated a favorable safety profile and was well-tolerated by the study participants. The treatment-related adverse events (TRAEs) observed during the study were predominantly mild to moderate in severity, classified as Grade 1 or 2. Notably, no Grade 3 or higher TRAEs were recorded, indicating the absence of severe treatment-related side effects.

The most frequently reported TRAEs included injection site reactions, chills, fatigue, and fever. These adverse events were generally mild and manageable, with no dose-limiting toxicities (DLTs) or treatment-related serious adverse events encountered throughout the study.

Table 12 provides a comprehensive summary of the TRAEs observed in the study, detailing the specific types of adverse events and their corresponding frequencies. This table allows for a clear understanding of the safety profile of AdV-HPV6/11 and supports the conclusion that the treatment is well-tolerated by patients with RRP.

The favorable safety profile of AdV-HPV6/11 is a promising finding, as it suggests that the treatment can be administered to RRP patients without significant risks or complications. This is particularly important for a patient population that often requires repeated surgical interventions, as a safe and effective alternative treatment could greatly improve their quality of life and reduce the burden of the disease.

TABLE 12
Treatment-Related Emergent Adverse
Events Occurring in >1 Patients.

| | Dose Level 1 | | Dose Level 2 | | |
|---|---|---|---|---|---|
| | (N = 3) | | (N = 35) | | |
| | Grade 1 | Grade 2 | Grade 1 | | Grade 2 |
| Event Term | (N, %) | (N, %) | (N, %) | | (N, %) |
| Chills | — | — | 25 | (71%) | — |
| Fatigue | — | — | 28 | (80%) | 2 (6%) |
| Fever | — | — | 24 | (69%) | — |
| Headache | | | 2 | (6%) | |
| Hyperhidrosis | — | — | 2 | (6%) | — |
| Injection site reaction | 3 (100%) | — | 34 | (97%) | — |
| Myalgia | — | — | 9 | (26%) | 2 (6%) |
| Nausea | — | — | 8 | (23%) | — |
| Vomiting | — | — | 2 | (6%) | — |

Treatment with AdV-HPV6/11 at dose level 2 demonstrated significant efficacy in patients with recurrent respiratory papillomatosis (RRP). The study results showed a 51% complete response rate, defined as no need for surgical intervention for 12 months after the completion of AdV-HPV6/11 treatment. This response rate was consistent across both the Phase 1 (n=12) and Phase 2 (n=23) portions of the study, with 50% and 52% complete response rates, respectively.

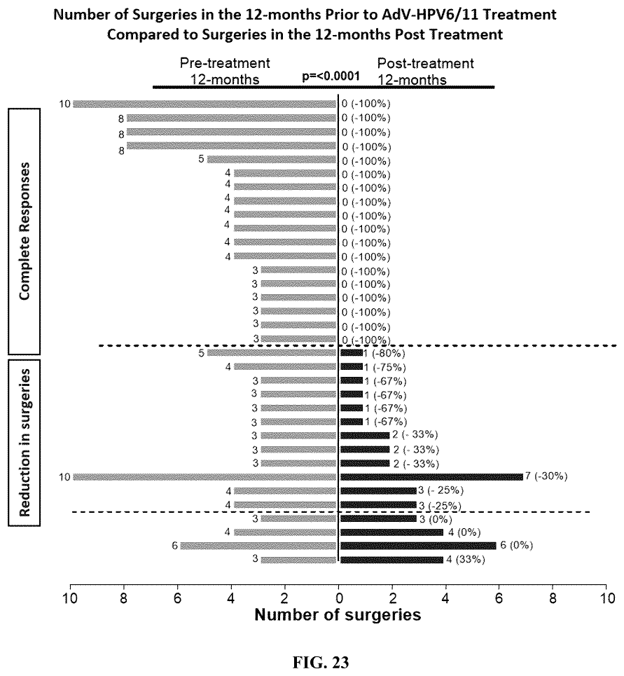

Moreover, 86% of the treated patients experienced a reduction in the number of surgeries required during the 12 months following AdV-HPV6/11 treatment completion compared to the 12 months prior to treatment. This reduction in the need for surgeries was observed in 83% of patients in the Phase 1 study and 87% of patients in the Phase 2 study, resulting in an average of 86% (30/35) of patients demonstrating a decreased need for surgical interventions (FIG. 23).

The AdV-HPV6/11 treatment at dose level 2 (n=35) also led to durable responses in the treated patients. Among the complete responders (n=18), long-term follow-ups were conducted to assess the duration of the response. The duration of response was defined as the time from the completion of AdV-HPV6/11 treatment until the date of the first surgery or the date of the last follow-up visit with no surgery.

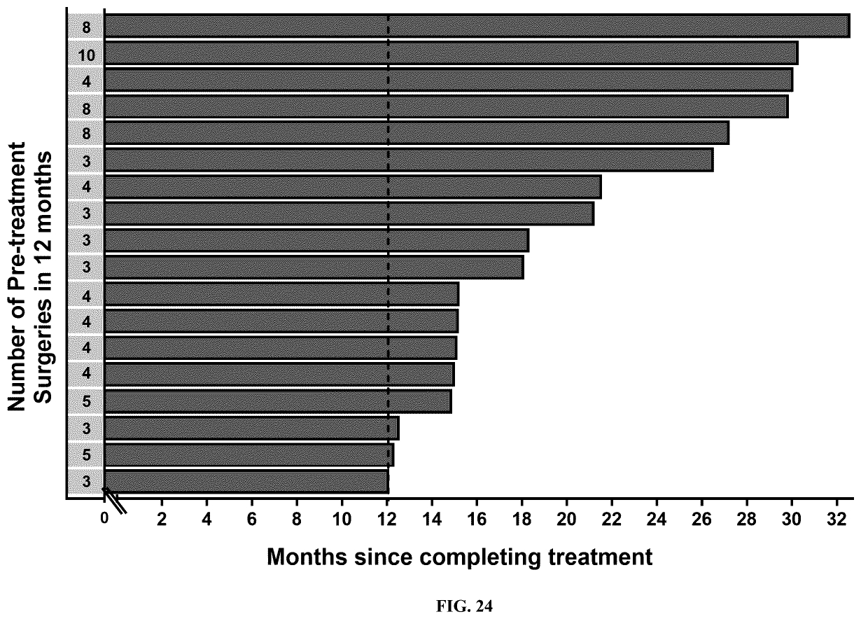

The median duration of follow-up was 20 months and is ongoing, indicating that the majority of patients continue to benefit from the treatment. The median duration of complete response has not yet been reached, as most patients' responses are still ongoing (FIG. 24). This finding suggests that the AdV-HPV6/11 treatment may provide long-lasting benefits for RRP patients, reducing the need for surgical interventions and improving their overall quality of life.

These results highlight the potential of AdV-HPV6/11 as an effective and durable treatment option for patients with RRP, offering a significant improvement over the current standard of care, which relies on repeated surgical procedures to manage the disease.

### Example 7: Constructing Variants of a Multi-Epitope Modular Fusion Protein/Antigen—Shuffling HPV Epitopes (Also Called Antigen Region)

Variant A (SEQ ID NO: 134), variant B (SEQ ID NO: 135), and variant C (SEQ ID NO: 136) are sequence variants of the multi-epitope modular fusion protein/antigen of SEQ ID NO: 68 in which the variant comprises the same HPV epitopes as SEQ ID NO: 68, but the HPV epitopes have been reordered.

In variant A, the HPV E2 epitope and the HPV11 E7 epitope were moved from the beginning of the fusion protein to the end of the multi-epitope modular fusion protein/antigen as compared to the multi-epitope module fusion protein/antigen of SEQ ID NO: 68.

In variant B, the HPV11 E7 epitope and the HPV E6 epitope were moved from the end of the multi-epitope module fusion protein/antigen to the beginning of the multi-epitope modular fusion protein/antigen as compared to the multi-epitope module fusion protein/antigen of SEQ ID NO: 68.

In variant C, the HPV E7 epitope, HPV11E7 epitope, and HPV E6 epitope were moved from the end of the multi-epitope module fusion protein/antigen to the beginning of the multi-epitope module fusion protein/antigen as compared to the multi-epitope module fusion protein/antigen of SEQ ID NO: 68, and HPV E2 epitope and the EVP11 E7 epitope were moved from the beginning of the multi-epitope module fusion protein/antigen to the end of the multi-epitope module fusion protein/antigen as compared to the multi-epitope module fusion protein/antigen of SEQ ID NO: 68.

### Example 8: Constructing Variants of a Multi-Epitope Modular Fusion Protein/Antigen—Fewer HPV Epitopes (Also Called Antigen Region)

Variant D (SEQ ID NO: 137), variant E (SEQ ID NO: 138), and variant F (SEQ ID NO: 139) are sequence variants of the multi-epitope modular fusion protein/antigen of SEQ ID NO: 68 in which the variant comprises fewer HPV epitopes.

Variant D lacks the second copy of the HPV11 E7 epitope that is the last HPV epitope in the multi-epitope modular fusion protein/antigen of SEQ ID NO: 68, and has a total of 10 HPV epitopes.

Variant E lacks the HPV6 E7 epitope compared to the multi-epitope modular fusion protein/antigen of SEQ ID NO: 68, and has a total of 10 HPV epitopes. In addition, variant E is a shuffle variant in which the HPV E2 epitope and the HPV11 E7 epitope were moved from the beginning of the fusion protein to the end of the multi-epitope modular fusion protein/antigen as compared to the multi-epitope module fusion protein/antigen of SEQ ID NO: 68.

Variant F lacks the 56 amino acid long HPV E4 epitope (SEQ ID NO: 3) and the 55 amino acid long HPV E4 (SEQ ID NO: 7) compared to the multi-epitope modular fusion protein/antigen of SEQ ID NO: 68, and has a total of 9 HPV epitopes. In addition, in variant F, the HPV11 E7 epitope and the HPV E6 epitope were moved from the end of the multi-epitope module fusion protein/antigen to the beginning of the multi-epitope modular fusion protein/antigen as compared to the multi-epitope module fusion protein/antigen of SEQ ID NO: 68.

### Example 9: Constructing Variants of a Multi-Epitope Modular Fusion Protein/Antigen—Additional HPV Epitopes (Also Called Antigen Region)

Variant G (SEQ ID NO: 140), variant H (SEQ ID NO: 141) and variant I (SEQ ID NO: 142) are sequence variants of the multi-epitope modular fusion protein/antigen of SEQ ID NO: 68 in which the variant comprises additional HPV epitopes.

Variant G contains an additional HPV11 E6 epitope added to the end of the multi-epitope modular fusion protein/antigen of SEQ ID NO: 68, and has a total of 12 HPV epitopes.

Variant H contains an additional HPV E7 epitope compared to shuffle variant C (SEQ ID NO: 136). Variant H contains the following epitopes, in order, HPVE6 E7 epitope, HPV E7 epitope, HPV11 E7 epitope, HPV E6 epitope, HPV E4 epitope (SEQ ID NO: 3), HPV11 E6 epitope, HPV6 E7 epitope, HPV6 E6 epitope, HPV E4 epitope (SEQ ID NO: 7), HPV11 E6 epitope, HPV E2 epitope, and HPV11 E7 epitope.

Variant I contains an additional HPV E4 epitope (SEQ ID NO: 3) and an additional HPV E7 epitope compared to the multi-epitope modular fusion protein/antigen of SEQ ID NO: 68, and has a total of 13 HPV epitopes. Variant I contains the following epitopes, in order, HPV E2 epitope, HPV11 E7 epitope, HPV E4 epitope (SEQ ID NO: 3), HPV11 E6 epitope, HPV E7 epitope, HPV E4 epitope (SEQ ID NO: 3), HPV6 E6 epitope, HPV E4 epitope (SEQ ID NO: 7), HPV11E6 epitope, HPV E7 epitope, HPV11 E7 epitope, HPV E7 epitope, and HPV E6 epitope.

### Example 10: Constructing Variants of a Multi-Epitope Modular Fusion Protein/Antigen—Conservative Substitutions in HPV Epitopes (Also Called Antigen Region)

Variant J (SEQ ID NO: 143) and Variant K (SEQ ID NO: 144) are sequence variants of the multi-epitope modular fusion protein/antigen of SEQ ID NO: 68 in which the variant comprises conservatively substituted amino acids in one or more HPV epitopes.

Variant J contains the conservative substitution of alanine to serine in the first antigen region (HPV E2 epitope). Specifically, all alanines are substituted with serines in the HPV E2 epitope.

Variant K contains two types of conservative substitutions. First, similar to variant J, all alanines are conservatively substituted with serines in the HPV E2 epitope (first antigen region). Second, all lysines are conservatively substituted with isoleucine in the second antigen region (HPV11 E7).

Additional variants of the multi-epitope modular fusion protein/antigen of SEQ ID NO: 68 may be generated so that the variants comprise one or more conservatively substituted amino acids in the first antigen region (HPV E2, SEQ ID NO: 105) of SEQ ID NO: 68. In these variants, the first antigen region may be any one of SEQ ID NO: 154 through SEQ ID NO: 160.

Variants of the multi-epitope modular fusion protein/antigen of SEQ ID NO: 68 may also be generated so that the variants comprise one or more conservatively substituted amino acids in the second antigen region (HPV11 E7, SEQ ID NO: 106) of SEQ ID NO: 68. In these variants, the second antigen region may be any one of SEQ ID NO: 164 through SEQ ID NO: 168.

Variants of the multi-epitope modular fusion protein/antigen of SEQ ID NO: 68 may also be generated so that the variants comprise one or more conservatively substituted amino acids in the third antigen region (HPV E4, SEQ ID NO: 107) of SEQ ID NO: 68. In these variants, the third antigen region may be any one of SEQ ID NO: 172 through SEQ ID NO: 176.

Variants of the multi-epitope modular fusion protein/antigen of SEQ ID NO: 68 may also be generated so that the variants comprise one or more conservatively substituted amino acids in the fourth antigen region (HPV11 E6, SEQ ID NO: 108) of SEQ ID NO: 68. In these variants, the fourth antigen region may be any one of SEQ ID NO: 180 through SEQ ID NO: 185.

Variants of the multi-epitope modular fusion protein/antigen of SEQ ID NO: 68 may also be generated so that the variants comprise one or more conservatively substituted amino acids in the fifth antigen region (HPV6 E7, SEQ ID NO: 109) of SEQ ID NO: 68. In these variants, the fifth antigen region may be any one of SEQ ID NO: 189 through SEQ ID NO: 193.

Variants of the multi-epitope modular fusion protein/antigen of SEQ ID NO: 68 may also be generated so that the variants comprise one or more conservatively substituted amino acids in the sixth antigen region (HPV6 E6, SEQ ID NO: 110) of SEQ ID NO: 68. In these variants, the sixth antigen region may be any one of SEQ ID NO: 197 through SEQ ID NO: 201.

Variants of the multi-epitope modular fusion protein/antigen of SEQ ID NO: 68 may also be generated so that the variants comprise one or more conservatively substituted amino acids in the seventh antigen region (HPV E4, SEQ ID NO: 111) of SEQ ID NO: 68. In these variants, the seventh antigen region may be any one of SEQ ID NO: 205 through SEQ ID NO: 209.

Variants of the multi-epitope modular fusion protein/antigen of SEQ ID NO: 68 may also be generated so that the variants comprise one or more conservatively substituted amino acids in the eighth antigen region (HPV11 E6, SEQ ID NO: 112) of SEQ ID NO: 68. In these variants, the eighth antigen region may be any one of SEQ ID NO: 213 through SEQ ID NO: 217.

Variants of the multi-epitope modular fusion protein/antigen of SEQ ID NO: 68 may also be generated so that the variants comprise one or more conservatively substituted amino acids in the ninth antigen region (HPV E7, SEQ ID NO: 113) of SEQ ID NO: 68. In these variants, the ninth antigen region may be any one of SEQ ID NO: 221 through SEQ ID NO: 225.

Variants of the multi-epitope modular fusion protein/antigen of SEQ ID NO: 68 may also be generated so that the variants comprise one or more conservatively substituted amino acids in the tenth antigen region (HPV11 E7, SEQ ID NO: 114) of SEQ ID NO: 68. In these variants, the tenth antigen region may be any one of SEQ ID NO: 229 through SEQ ID NO: 233.

Variants of the multi-epitope modular fusion protein/antigen of SEQ ID NO: 68 may also be generated so that the variants comprise one or more conservatively substituted amino acids in the eleventh antigen region (HPV E6, SEQ ID NO: 115) of SEQ ID NO: 68. In these variants, the eleventh antigen region may be any one of SEQ ID NO: 237 through SEQ ID NO: 242.

### Example 11: Constructing Variants of a Multi-Epitope Modular Fusion Protein/Antigen—Amino Acid Additions and Deletions within HPV Epitopes (Also Called Antigen Region)

Variant L (SEQ ID NO: 145) and Variant M (SEQ ID NO: 136) are sequence variants of the multi-epitope modular fusion protein/antigen of SEQ ID NO: 68 in which the variant comprises one or more additional amino acids in one or more HPV epitopes.

Variant L contains 3 additional amino acids (VDG) at the C-terminal end of the second antigen region (HPV11 E7) to generate a variant HPV11 E7 epitope having SEQ ID NO: 149 (HCCYEQLEDSSEIDEVDG), but otherwise Variant L has the same order of HPV epitopes as SEQ ID NO: 68.

Variant M contains 5 additional amino acids (SEQ ID NO: 150: NLKVV) at the C-terminal end of the fourth antigen region (HPV11 E6 epitope) to generate a variant HPV11 E6 epitope having SEQ ID NO: 151 (TAEIYAYVQTNHGLIAYKNLKVV), but otherwise Variant M has the same order of HPV epitopes as SEQ ID NO: 68.

Variant N (SEQ ID NO: 147) and Variant O (SEQ ID NO: 148) are variants of the multi-epitope modular fusion protein/antigen of SEQ ID NO: 68 in which the variant comprises a deletion of one or more amino acids in one or more HPV epitopes.

Variant N has a deletion of 2 amino acids from the fourth antigen region (HPV11 E6 epitope) in amino acid positions 8-9 (AY deleted) to generate a variant HPV11 E6 epitope having SEQ ID NO: 152 (TAEIYAYKNLKVV), but otherwise Variant N has the same order of HPV epitopes as SEQ ID NO: 68.

Variant O has a deletion of 4 amino acids from the seventh antigen region (HPV E4, SEQ ID NO: 3) at amino acid position 7 (G deleted), 25-26 (LD deleted), and 43 (S deleted) to generate a variant HPV E4 epitope having SEQ ID NO: 153 (QCKRRLNEHEESNSPLATPCVWPDPWTVETTTSSLTITTTKDGTTVTVQLR), but otherwise Variant O has the same order of HPV epitopes as SEQ ID NO: 68.

Additional variants of the multi-epitope modular fusion protein/antigen of SEQ ID NO: 68 may be generated so that the variants contain one or more amino acid deletions in the first antigen region (HPV E2, SEQ ID NO: 105) of SEQ ID NO: 68. In these variants, the first antigen region may be any one of SEQ ID NO: 161 through SEQ ID NO: 163.

Variants of the multi-epitope modular fusion protein/antigen of SEQ ID NO: 68 may also be generated so that the variants contain one or more amino acid deletions in the second antigen region (HPV11 E7, SEQ ID NO: 106) of SEQ ID NO: 68. In these variants, the second antigen region may be any one of SEQ ID NO: 169 through SEQ ID NO: 171.

Variants of the multi-epitope modular fusion protein/antigen of SEQ ID NO: 68 may also be generated so that the variants contain one or more amino acid deletions in the third antigen region (HPV E4, SEQ ID NO: 107) of SEQ ID NO: 68. In these variants, the third antigen region may be any one of SEQ ID NO: 177 through SEQ ID NO: 179.

Variants of the multi-epitope modular fusion protein/antigen of SEQ ID NO: 68 may also be generated so that the variants contain one or more amino acid deletions in the fourth antigen region (HPV11 E6, SEQ ID NO: 108) of SEQ ID NO: 68. In these variants, the fourth antigen region may be any one of SEQ ID NO: 186 through SEQ ID NO: 188.

Variants of the multi-epitope modular fusion protein/antigen of SEQ ID NO: 68 may also be generated so that the variants contain one or more amino acid deletions in the fifth antigen region (HPV6 E7, SEQ ID NO: 109) of SEQ ID NO: 68. In these variants, the fifth antigen region may be any one of SEQ ID NO: 194 through SEQ ID NO: 196.

Variants of the multi-epitope modular fusion protein/antigen of SEQ ID NO: 68 may also be generated so that the variants contain one or more amino acid deletions in the sixth antigen region (HPV6 E6, SEQ ID NO: 110) of SEQ ID NO: 68. In these variants, the sixth antigen region may be any one of SEQ ID NO: 202 through SEQ ID NO: 204.

Variants of the multi-epitope modular fusion protein/antigen of SEQ ID NO: 68 may also be generated so that the variants contain one or more amino acid deletions in the seventh antigen region (HPV E4, SEQ ID NO: 111) of SEQ ID NO: 68. In these variants, the seventh antigen region may be any one of SEQ ID NO: 210 through SEQ ID NO: 212.

Variants of the multi-epitope modular fusion protein/antigen of SEQ ID NO: 68 may also be generated so that the variants contain one or more amino acid deletions in the eighth antigen region (HPV11 E6, SEQ ID NO: 112) of SEQ ID NO: 68. In these variants, the eighth antigen region may be any one of SEQ ID NO: 218 through SEQ ID NO: 220.

Variants of the multi-epitope modular fusion protein/antigen of SEQ ID NO: 68 may also be generated so that the variants contain one or more amino acid deletions in the ninth antigen region (HPV E7, SEQ ID NO: 113) of SEQ ID NO: 68. In these variants, the ninth antigen region may be any one of SEQ ID NO: 226 through SEQ ID NO: 228.

Variants of the multi-epitope modular fusion protein/antigen of SEQ ID NO: 68 may also be generated so that the variants contain one or more amino acid deletions in the tenth antigen region (HPV11 E7, SEQ ID NO: 114) of SEQ ID NO: 68. In these variants, the tenth antigen region may be any one of SEQ ID NO: 234 through SEQ ID NO: 236.

Variants of the multi-epitope modular fusion protein/antigen of SEQ ID NO: 68 may also be generated so that the variants contain one or more amino acid deletions in the eleventh antigen region (HPV E6, SEQ ID NO: 115) of SEQ ID NO: 68. In these variants, the eleventh antigen region may be any one of SEQ ID NO: 243 through SEQ ID NO: 246.

### Example 12: Manufacture of a Recombinant Gorilla Adenovirus Vaccine Expressing Variant(s) of a Multi-Epitope Modular Fusion Protein/Antigen

In this example, vaccines expressing variants of the multi-epitope modular fusion protein/antigen (SEQ ID NO: 68) are developed using the gorilla adenoviral vector GC46. The variants of SEQ ID NO: 68 are described in Examples 7-10. GC46 is isolated from the stool of a healthy African gorilla and is rendered replication-incompetent by deleting the E1 and E4 genes. Additionally, the bovine growth hormone (BGH) poly A signal sequence is inserted in place of the E4 open reading frames (ORFs) to stop transcription initiated from the retained E4 promoter.

The GC46 vector is further engineered to express a variant of SEQ ID NO: 68 under the control of a cytomegalovirus (CMV) immediate early promoter-driven expression cassette. The antigen expression cassette is located at the E1 region deletion junction and is oriented right-to-left with respect to the GC46 viral genome. The CMV enhancer/promoter controls the initiation of transcription and includes the viral enhancer CAAT box, TATA box, transcription start site, and 5′ splice site sequences.

The expression cassette sequences are followed by an artificial untranslated region (UTR) containing a splice donor sequence, 3′ splice site sequences, the open reading frame of the gene to be expressed, and the SV40 early polyadenylation and transcriptional termination signal sequences. The tetracycline response element (TRE) is positioned between the TATA box and the transcription initiation site. When this adenovector is produced in cell lines expressing the Tet repressor, the expression of the CMV-driven transgene is reduced.

The manufacturing process for these vaccines is similar to that described in Example 1 for the vaccine expressing the multi-epitope modular fusion protein/antigen of SEQ ID NO: 68. The key differences are the use of the GC46 adenoviral vector and the incorporation of variants of SEQ ID NO: 68 described in Examples 7-10. The modifications made to the GC46 vector, including the deletion of E1 and E4 genes, insertion of the BGH poly A signal sequence, and the addition of the CMV-driven expression cassette, ensure the safe and efficient expression of the desired antigen variants in the target cells.

### Example 13: Treatment with Variants of AdV-HPV6/11 Result in Improved Derkay Score in RRP Patients

Subjects with RRP are treated with a vaccine expressing a variant of SEQ ID NO: 68, as described in Examples 7-10, following the methodology outlined in Examples 5-6. This “variant vaccine” is administered to patients at two dose levels: dose level 1 (1×10¹¹ PU) and dose level 2.

The treatment regimen consists of four subcutaneous injections, one in each limb, administered over a period of 12 weeks. The first injection is given immediately after a surgical papilloma cleanout procedure. Up to two additional papilloma removal procedures are performed as needed during the 12-week vaccination period to maintain minimal residual disease.

To evaluate the clinical response to the variant vaccine, the number of clinically indicated interventions in the 12 months following the study treatment is compared to the number of interventions in the 12 months preceding the study treatment. All patients who receive the variant vaccine at dose level 1 experience a decrease in the number of interventions in the 12 months following the completion of the study treatment.

The clinical data also demonstrate that the variant vaccine treatment significantly reduces the need for surgeries in patients with severe, aggressive RRP who are treated at dose level 2. At this dose level, at least 50% of the patients achieve a Complete Response, and the Overall Response Rate is at least 50%. Furthermore, more than 80% of the patients treated at dose level 2 require fewer surgeries following treatment with the variant vaccine.

In addition to the reduction in surgical interventions, treatment with the variant vaccine significantly improves anatomical Derkay scores, which are used to assess the severity of RRP. The variant vaccine also induces HPV-specific T cell responses that are capable of trafficking into the papilloma microenvironment, suggesting an immunological mechanism for the observed clinical benefits.

These results highlight the potential of the variant vaccine as an effective treatment option for patients with RRP, particularly those with severe and aggressive disease. The significant reduction in the need for surgical interventions and the improvement in anatomical scores demonstrate the clinical efficacy of the variant vaccine, while the induction of HPV-specific T cell responses provides insight into the immunological basis for these therapeutic effects.

### Example 14: Treating RRP with AdV-HPV6/11 in Combination with an Angiogenesis Inhibitor

Subjects with RRP are treated with a combination therapy consisting of AdV-HPV6/11 (or a variant disclosed herein) and Bevacizumab (AVASTIN®). The AdV-HPV6/11 vaccination is administered as described in the previous examples, followed by the administration of Bevacizumab.

The combination therapy works through two complementary mechanisms. First, AdV-HPV6/11 primes the patient's immune system to recognize and target HPV6/11-infected cells. Second, Bevacizumab, a monoclonal antibody, specifically targets and inhibits vascular endothelial growth factor (VEGF), a key signaling molecule involved in the formation of new blood vessels (angiogenesis).

By inhibiting VEGF signaling pathways, Bevacizumab helps to slow down the growth and progression of RRP by limiting the blood supply to the papillomas. This effect, combined with the immune system priming by AdV-HPV6/11, creates a synergistic and targeted attack on HPV6/11-infected cells, effectively slowing or preventing the growth of RRP-associated papillomas.

Patients treated with the combination of AdV-HPV6/11 and Bevacizumab demonstrate significant clinical improvements, as evidenced by a marked reduction in their Derkay scores, which are used to assess the severity and extent of RRP lesions. Additionally, this combination therapy leads to an expansion of peripheral HPV-specific T cell responses, indicating a robust and potent antigen spreading effect against HPV6/11.

The successful treatment of RRP patients with AdV-HPV6/11 in combination with Bevacizumab highlights the potential of this approach to effectively target and eliminate HPV6/11-infected cells while simultaneously inhibiting the growth and spread of RRP-associated papillomas. This combination therapy represents a promising strategy for managing and treating RRP.

### Example 15: Treating RRP with AdV-HPV6/11 in Combination with a PD-1 Inhibitor

Subjects with recurrent respiratory papillomatosis (RRP) are treated with a combination therapy consisting of AdV-HPV6/11 (or a variant disclosed herein) and Pembrolizumab (KEYTRUDA®). The AdV-HPV6/11 vaccination is administered as described in the previous examples, followed by the administration of Pembrolizumab.

The combination therapy works through two complementary mechanisms. First, AdV-HPV6/11 primes the patient's immune system to recognize and target HPV6/11-infected cells. Second, Pembrolizumab, a monoclonal antibody, specifically targets and blocks the programmed cell death protein 1 (PD-1) receptor on T cells.

By binding to the PD-1 receptor and preventing its interaction with PD-L1 and PD-L2, which are often expressed on tumor cells and other cells in the tumor microenvironment, Pembrolizumab reactivates T cells and enhances their ability to recognize and attack HPV6/11-positive cells more effectively. This synergistic effect leads to a more potent and targeted attack on HPV6/11-infected cells.

Patients treated with the combination of AdV-HPV6/11 and Pembrolizumab demonstrate significant clinical improvements, as evidenced by a marked reduction in their Derkay scores, which are used to assess the severity and extent of RRP lesions. Additionally, this combination therapy leads to an expansion of peripheral HPV-specific T cell responses, indicating a robust and potent antigen spreading effect against HPV6/11.

The successful treatment of RRP patients with AdV-HPV6/11 in combination with Pembrolizumab highlights the potential of this approach to effectively target and eliminate HPV6/11-infected cells by enhancing the immune system's ability to recognize and attack these cells. This combination therapy represents a promising strategy for managing and treating RRP.

### Example 16: Treating RRP with AdV-HPV6/11 in Combination with a PD-L1 Inhibitor

Subjects with recurrent respiratory papillomatosis (RRP) caused by human papillomavirus types 6 and 11 (HPV6/11) are treated with a combination therapy consisting of AdV-HPV6/11 (or a variant disclosed herein) and Atezolizumab (TECENTRIQ®). The AdV-HPV6/11 vaccination is administered as described in the previous examples, followed by the administration of Atezolizumab.

The combination therapy works through two complementary mechanisms. First, AdV-HPV6/11 primes the patient's immune system to recognize and target HPV6/11-infected cells. Second, Atezolizumab, a monoclonal antibody, specifically binds to PD-L1, preventing it from interacting with PD-1 and CD80 receptors on T cells.

By blocking the interaction between PD-L1 and its receptors, Atezolizumab lifts the inhibitory signals on T cells and restores their ability to recognize and attack HPV6/11-positive cells. This synergistic effect leads to a more potent and targeted attack on HPV6/11-infected cells.

Patients treated with the combination of AdV-HPV6/11 and Atezolizumab demonstrate significant clinical improvements, as evidenced by a marked reduction in their Derkay scores, which are used to assess the severity and extent of RRP lesions. Additionally, this combination therapy leads to an expansion of peripheral HPV-specific T cell responses, indicating a robust and potent antigen spreading effect against HPV6/11.

The successful treatment of RRP patients with AdV-HPV6/11 in combination with Atezolizumab highlights the potential of this approach to effectively target and eliminate HPV6/11-infected cells by enhancing the immune system's ability to recognize and attack these cells. This combination therapy represents a promising strategy for managing and treating RRP.

### Example 17: Cross-Coverage Analysis of AdV-HPV6/11

MHC-I and MHC-II epitopes were predicted in the provided ADV-HPV6/11 protein sequence with MHC binding prediction algorithms in a local installation of IEDB tools. For MHC-I epitopes, the IEDB recommended 2020.09 method (NetMHCpan EL 4.1) was used. The epitope length range was 8-10 AA; the prediction was done in the IEDB HLA allele reference set of the 27 most common MHC-I alleles.

For MHC-II epitopes, the IEDB recommended 2.22 method was employed; this method uses a consensus approach, combining NN-align 2.3, SMM-align 1.1, CombLib 1.0, and Sturniolo 1.0 if any corresponding predictor is available for the MHC allele, otherwise NetMHCIIpan 3.2 is used. The epitope length range was 12-15 and 19-22 AA; the prediction was done in the IEDB HLA allele reference set of the 27 most common MHC-II alleles.

Representative AdV-HPV6/11 epitopes were selected based on their binding prediction ranks (MHC-I: percentile rank <=1; MHC-II: percentile rank <=10), removing any completely overlapping redundant sequences resulting from different epitope lengths in each class and only keeping the longest epitope sequences.

The representative AdV-HPV6/11 epitope sequences were aligned to protein sequences of HPV genotypes 6 and 11 that had been downloaded from NCBI (https://www.ncbi.nlm.nih.gov/protein/), using blastp in a local BLAST installation, optimized for short input sequences. Representative AdV-HPV6/11 epitopes that yielded BLAST alignments with >75% overall identity to any of the HPV proteins (calculated from % coverage×% identity of the covered sequence) were considered for further evaluation.

Furthermore, MHC-I and MHC-II epitope predictions were performed in HPV-6 and HPV-11 for HPV reference protein sequences downloaded from PaVE (https://pave.niaid.nih.gov/), with the same methods and parameters as in ADV-HPV6/11. Predicted HPV-6 and HPV-11 epitopes were compared with representative AdV-HPV6/11 epitopes, using an in-house developed script.

IEDB-predicted epitopes in HPV-6 and HPV-11 reference protein sequences were considered covered by AdV-HPV6/11 if their sequence was completely contained without mismatch in HPV peptides of the respective HPV genotypes that aligned to a representative AdV-HPV6/11 epitope with >75% overall identity and had themselves >75% sequence identity to at least one representative AdV-HPV6/11 epitope. Representative HPV-6 and HPV-11 epitopes were selected based on their binding prediction ranks (MHC-I: percentile rank <=1; MHC-II: length-adjusted percentile rank <=10; this selected strong binders), removing any completely overlapping redundant sequences resulting from different epitope lengths in each class and only keeping the longest epitope sequences.

AdV-HPV6/11 coverage of MHC-I and MHC-II epitopes was predicted in both analyzed HPV genotypes. Coverage of HPV-6 was higher than that of HPV-11, as could be expected based on the proportions of AdV-HPV6/11 sequence that originate from HPV-6 and HPV-11, respectively. The relative coverage differences increased with increasing sequence identity thresholds of >75%, >=90%, and 100% (Tables 10, 11). Good overall sequence similarity of HPV-6-derived ADV-HPV6/11 sequences to HPV-11, and therefore potential to confer immunoreactivity also to HPV-11, is reflected in the fact that a higher proportion of ADV-HPV6/11 sequence was covered by strongly binding HPV-11 epitopes at all thresholds than originated from HPV-11. Differences between HPV-6 and HPV-11 in coverage with overlapping representative epitopes (2+ or more) were more pronounced.

Table 10, below, shows predicted MHC-I epitope coverage by AdV-HPV6/11. Percentages of AdV-HPV6/11 sequence covered by strongly binding HPV-6 and HPV-11 epitopes are shown at different sequence similarity thresholds. Coverage with a minimum number of 2 overlapping representative epitopes is indicated by epitope coverage of 2+; analogous for higher numbers.

TABLE 10
Prediction of MHC-I Epitope Coverage by AdV-HPV6/11

| | HPV-6 | | | HPV-11 | | |
|---|---|---|---|---|---|---|
| | Sim. >75% | Sim. >=90% | Sim. 100% | Sim. >75% | Sim. >=90% | Sim. 100% |
| Epitope | % of | % of | % of | % of | % of | % of |
| coverage | length | length | length | length | length | length |
| 0 | 17.1 | 18.7 | 20.2 | 35.7 | 58.5 | 75.1 |
| 1+ | 82.9 | 81.3 | 79.8 | 64.3 | 41.5 | 24.9 |
| 2+ | 68.6 | 66.6 | 63 | 36.2 | 19.8 | 9.4 |
| 3+ | 46.6 | 45 | 42.6 | 16.7 | 8.5 | 3.8 |
| 4+ | 24.1 | 22 | 22 | 7.1 | 4 | 2.7 |
| 5+ | 8.7 | 8.5 | 8.5 | 2 | 1.6 | 1.4 |
| 6+ | 1.8 | 1.6 | 1.6 | 0.1 | 0.1 | 0.1 |
| 7 | 0.3 | 0.3 | 0.3 | 0 | 0 | 0 |

Table 11, below, shows predicted MHC-II epitope coverage by AdV-HPV6/11. Percentages of AdV-HPV6/11 sequence covered by strongly binding HPV-6 and HPV-11 epitopes are shown at different sequence similarity thresholds. Coverage with a minimum number of 2 overlapping representative epitopes is indicated by Epitope coverage of 2+; analogous for higher numbers.

TABLE 11
Prediction of MHC-II Epitope Coverage by AdV-HPV6/11

| | HPV-6 | | | HPV-11 | | |
|---|---|---|---|---|---|---|
| | Sim. >75% | Sim. >=90% | Sim. 100% | Sim. >75% | Sim. >=90% | Sim. 100% |
| Epitope | % of | % of | % of | % of | % of | % of |
| coverage | length | length | length | length | length | length |
| 0 | 28.1 | 29 | 34.9 | 36.7 | 64.7 | 88.9 |
| 1+ | 71.9 | 71 | 65.1 | 63.3 | 35.3 | 11.1 |
| 2+ | 68 | 65.6 | 60.4 | 55.1 | 23.1 | 6.6 |
| 3+ | 63.8 | 61.7 | 56.4 | 47.8 | 14.7 | 5.8 |
| 4+ | 60.4 | 58.5 | 49.1 | 40.1 | 10.2 | 5.1 |
| 5+ | 56 | 54.4 | 45.3 | 32.6 | 8.6 | 4.2 |
| 6+ | 49.3 | 47.9 | 38.9 | 24.8 | 7.2 | 1.3 |
| 7+ | 43.9 | 42.7 | 34.4 | 17.1 | 4.2 | 0 |
| 8+ | 36.3 | 36.3 | 29 | 12.4 | 3.4 | 0 |
| 9+ | 30.1 | 30.1 | 23.9 | 10.2 | 2 | 0 |
| 10+ | 23.3 | 23.3 | 19 | 8.2 | 0.9 | 0 |
| 11+ | 16.7 | 16.7 | 14.2 | 5.7 | 0.6 | 0 |
| 12+ | 11.8 | 11.8 | 10.5 | 3.8 | 0 | 0 |
| 13+ | 5.4 | 5.4 | 5.3 | 1.9 | 0 | 0 |
| 14+ | 3.5 | 3.5 | 3.5 | 0.3 | 0 | 0 |
| 15 | 1 | 1 | 1 | 0 | 0 | 0 |

ADV-HPV6/11 coverage of MHC-J and MHC-JJ epitopes was predicted in both analyzed HPV genotypes. AdV-HPV6/11 epitope coverage profiles were generated by counting overlaps of representative HPV-6 and HPV-11 epitopes with ADV-HPV6/11 antigen region positions at different sequence similarity thresholds. Largely due to length differences between MHC-J and MHC-JJ epitopes, observed peaks of MHC-JJ coverage were higher on average than MHC-J coverage peaks. Coverage with HPV-11 epitopes was also observed in large parts of HPV-6-derived ADV-HPV6/11 antigen regions, not only HPV-11 derived regions.

Average and median coverage with HPV-6 epitopes was higher than with HPV-11 epitopes; the difference increased with increasing sequence similarity thresholds. Decreases in coverage with increasing similarity threshold were more pronounced in HPV-11 than in HPV-6, as can be expected, as >92% of the AdV-HPV6/11 sequence derives from HPV-6.

Ultimately, the foregoing results suggest that AdV-HPV6/11 can confer immunity to HPV-6 and HPV-11 in similar measure, but with a considerably better therapeutic effect against HPV-6 than HPV-11 infections.

### SEQUENCE LISTING

SEQUENCE ID NOs. 1-81 disclosed in WO 2022/115470 A1 are hereby incorporated by reference as if fully and expressly set forth herein as the same. SEQUENCE ID NOs. 82-148 (some of which may be duplicative of SEQUENCE ID NOs. 1-81) are as follows:

Description of key regions of AdV-HPV6/11

| | | | | SE- |
|---|---|---|---|---|
| | Size | | | QUENCE |
| Location | (bp) | Element | Function | ID NO. |
| 1-458 | 458 | ITR and | Inverted terminal repeat, | 82 |
| | | Packaging | serves as starting point for | |
| | | domain | viral replication, and | |
| | | | packaging domain controls | |
| | | | for the | |
| | | | packaging/encapsidation | |
| | | | of the viral genome to | |
| | | | form a mature viral | |
| | | | particle | |
| 483-663 | 181 | SV40 | Part of transgene cassette, | 83 |
| | | poly A | inserted into deleted E1 | |
| | | and | region, aids in | |
| | | 3′UTR | transcription termination, | |
| | | | export of mRNA from the | |
| | | | nucleus. | |
| 664-3039 | 2376 | AdV- | Part of the transgene | 84 |
| | | HPV6/11 | cassette, inserted into | |
| | | antigen | deleted E1 regions, | |
| | | ORF | encodes for the AdV- | |
| | | | HPV6/11 antigen protein | |
| | | | expressed and includes the | |
| | | | start and stop codon of the | |
| | | | ORF | |
| 3040-3959 | 920 | CMV | Part of transgene cassette, | 85 |
| | | promoter | inserted into deleted E1 | |
| | | | region, transcriptional | |
| | | | promoter element which | |
| | | | includes the transcriptional | |
| | | | donor and acceptor splice | |
| | | | sites and 5′ UTR | |
| 5680-10973 | 5294 | E2B | Encodes for proteins | 86 |
| | | | involved in viral | |
| | | | replication, contains the | |
| | | | genes for DNA | |
| | | | polymerase and precursor | |
| | | | terminal protein | |
| 11471-14510 | 3040 | L1 | Encodes for structural | 87 |
| | | | proteins including | |
| | | | encapsidation and capsid | |
| | | | protein precursors | |
| 14582-18606 | 4025 | L2 | Encodes for structural | 88 |
| | | | proteins including the | |
| | | | penton base | |
| 18706-23083 | 4378 | L3 | Encodes for structural | 89 |
| | | | proteins including hexon | |
| | | | and core proteins | |
| Anti- | 1638 | E2A | Encodes for proteins | 90 |
| gen23176- | | | involved in replication of | |
| 24813 | | | viral DNA | |
| 24857-28701 | 3845 | L4 | Encodes for structural | 91 |
| | | | proteins including hexon | |
| | | | assembly proteins and | |
| | | | capsid protein precursors | |
| 28702-32589 | 3888 | E3 | Encodes for proteins | 92 |
| | | | involved in protecting the | |
| | | | virus from host antiviral | |
| | | | responses | |
| 32792-34534 | 1743 | L5 | Encodes for fiber protein | 93 |
| 35275-35358 | 84 | ITR | Inverted Terminal Repeat, | 94 |
| | | | serves as starting points | |
| | | | for viral replication | |

CMV—Cytomegalovirus,
ORF—Open Reading Frame,
Poly A—Polyadenylic Acid,
SV40—Simian Virus 40,
UTR—Untranslated Region

Location and sequence of AdV-HPV6/11 E4 spacer

| Location | | SEQ ID |
|---|---|---|
| (bp) | Nucleotide Sequence | NO. |
| 34692- | CAATTGACGGCCGCCATAGAGCCCACCGCATCCCCAGCATGCCT | 95 |
| 34969 | GCTATTGTCTTCCCAATCCTCCCCCTTGCTGTCCTGCCCCACCCC | |
| | ACCCCCCAGAATAGAATGACACCTACTCAGACAATGCGATGCA | |
| | ATTTCCTCATTTTATTAGGAAAGGACAGTGGGAGTGGCACCTTC | |
| | CAGGGTCAAGGAAGGCACGGGGGAGGGGCAAACAACAGATGG | |
| | CTGGCAACTAGAAGGCACAGGGTACCAACGCGTCGTAACTATA | |
| | ACGGTCCTAAGGTAGCG | |

Key regions of AdV-HPV6/11 expression cassette/payload

| | Identi- | Size | | SEQUENCE |
|---|---|---|---|---|
| Element | fication | (bp) | Nucleotide Sequence | ID NO. |
| CMV enhancer | MOD- | 365 | CTAGTTATTAATAGTAATCAAT | 96 |
| | 1763396 | | TACGGGGTCATTAGTTCATAGC | |
| | | | CCATATATGGAGTTCCGCGTTA | |
| | | | CATAACTTACGGTAAATGGCCC | |
| | | | GCCTGGCTGACCGCCCAACGAC | |
| | | | CCCCGCCCATTGACGTCAATAA | |
| | | | TGACGTATGTTCCCATAGTAAC | |
| | | | GCCAATAGGGACTTTCCATTGA | |
| | | | CGTCAATGGGTGGAGTATTTAC | |
| | | | GGTAAACTGCCCACTTGGCAGT | |
| | | | ACATCAAGTGTATCATATGCCA | |
| | | | AGTACGCCCCCTATTGACGTCA | |
| | | | ATGACGGTAAATGGCCCGCCTG | |
| | | | GCATTATGCCCAGTACATGACC | |
| | | | TTATGGGACTTTCCTACTTGGC | |
| | | | AGTACATCTACGTATTAGTCAT | |
| | | | CGCTATTACCATG | |
| CMV promoter | MOD- | 205 | GTGATGCGGTTTTGGCAGTACA | 97 |
| | 1763400 | | TCAATGGGCGTGGATAGCGGTT | |
| | | | TGACTCACGGGGATTTCCAAGT | |
| | | | CTCCACCCCATTGACGTCAATG | |
| | | | GGAGTTTGTTTTGGCACCAAAA | |
| | | | TCAACGGGACTTTCCAAAATGT | |
| | | | CGTAACAACTCCGCCCCATTGA | |
| | | | CGCAAATGGGCGGTAGGCGTGT | |
| | | | ACGGTGGGAGGTCTATATAAGC | |
| | | | AGAGCTC | |
| 2X TetO | MOD- | 42 | TCCCTATCAGTGATAGAGATCT | 98 |
| | 1763401 | | CCCTATCAGTGATAGAGATC | |
| TSS + 5′ UTR | MOD- | 295 | GTCGACGAGCTCGTTTAGTGAA | 99 |
| with splice unit | 1763404 | | CCGTCAGATCGCCTGGAGACGC | |
| | | | CATCCACGCTGTTTTGACCTCC | |
| | | | ATAGAAGACACCGGGACCGAT | |
| | | | CCAGCCTCCGCGGCCGGGAACG | |
| | | | GTGCATTGGAACGCGGATTCCC | |
| | | | CGTGCCAAGAGTGACGTAAGTA | |
| | | | CCGCCTATAGAGTCTATAGGCC | |
| | | | CACCCCCTTGGCTTCACTTGAG | |
| | | | GTGTGGCAGGCTTGAGATCTGG | |
| | | | CCATACACTTGAGTGACAATGA | |
| | | | CATCCACTTTGCCTTTCTCTCCA | |
| | | | CAGGTGTCCACTCCCAGGTCCA | |
| | | | ACTGCAGCC | |
| KpnI | MOD- | 6 | GGTACC | |
| Restriction Site | 944 | | | |
| Kozak | MOD- | 7 | CGCCACC | |
| | 213794 | | | |
| Antigen | MOD- | 2376 | ATGGAAGCCATTGCCAAAAGA | 102 |
| | 1761058 | | CTCGACGCTTGCGCCGAGCAGC | |
| | | | TGCTGGAGCTGGCCGAAGAGA | |
| | | | ATAGCACAGACCTCCACAAACA | |
| | | | CGTCCTGCATTGGAAGTGCATG | |
| | | | AGACATGCCTCCGTGCTCCTGT | |
| | | | ATAAGGCCAAGCAGATGGGAC | |
| | | | TGTCCCACATTGGAATGCAGGT | |
| | | | CGTGCCTCCCCTCAAGGTCAGC | |
| | | | GAAGCCAAAGGCCATAACGCT | |
| | | | ATCGAAATGCAAATGCACCTGG | |
| | | | AAAGCCTCCTGAGGACCGAATA | |
| | | | CTCCATGGAACCCTGGACCCTC | |
| | | | CAGGAAACCTCCTACGCTATGT | |
| | | | GGCAGACACCCCCTAAGAGGT | |
| | | | GCTTTAAGAAAAGAGGAAAGA | |
| | | | CAGTGGAAGTGAAATTCGATGG | |
| | | | CTGTGCCAATAACACAATGGAT | |
| | | | TACGTCGTGTGGACCGATGTGT | |
| | | | ATGTGCAAGACAATGACACATG | |
| | | | GGTCAAGGTCCACTCCATGGTG | |
| | | | GACGCTAAGGGAATCTATTACA | |
| | | | CATGCGGACAGTTTAAGACATA | |
| | | | CTATGTGAATTTCGTCAAGGAA | |
| | | | GCCGAAAAGTATGGCTCCACCA | |
| | | | AACACTGGGAGGTCTGCTATGG | |
| | | | CTCCACCGTCATCTGTAGCCCT | |
| | | | GCCTCCGTGTCCAGCACAACCC | |
| | | | AAGAGGTCAGCATTCCCGAAA | |
| | | | GCACAACCTATACCCCTGCCCA | |
| | | | GACCTCCACCCTCGTGTCCAGC | |
| | | | TCCACCAAAGAGGATGCCGTCC | |
| | | | AGACACCCCCTAGAAAAAGAG | |
| | | | CTAGAGGAGTGCAACAGTCCCC | |
| | | | CTGTAACGCTCTGTGTGTGGCT | |
| | | | CACATTGGCCCTGTGGATAGCG | |
| | | | GAAACCATAACCTCATCACAAA | |
| | | | CAATCACGATCAGCATCAGAGG | |
| | | | AGAAATAACTCCAACTCCAGCG | |
| | | | CTACCCCTATCGTCCAGTTTCA | |
| | | | GGGAGAGTCCAACTGTCTGAAA | |
| | | | TGCTTTAGATATAGACTCAACG | |
| | | | ATAGACATAGACATCTGTTTGA | |
| | | | CCTCATCTCCAGCACATGGCAT | |
| | | | TGGGCCAGCTCCAAGGCTCCCC | |
| | | | ATAAGCACGCCATTGTGACAGT | |
| | | | GACATACGATAGCGAAGAGCA | |
| | | | AAGACAACAGTTTCTGGATGTG | |
| | | | GTCAAGATTCCCCCTACCATTA | |
| | | | GCCATAAGCTCGGCTTTATGTC | |
| | | | CCTGCATCTGCTCCATTGCTAT | |
| | | | GAGCAACTGGAAGACTCCAGC | |
| | | | GAAGACGAAGTGGATATGGCA | |
| | | | GACGATTCTGCACTGCATAAAA | |
| | | | AGTACCCATTCCTGAACCTGCT | |
| | | | GCATACCCCACCGCATCGCCCA | |
| | | | CCGCCACTGTGTCCGCAAGCTC | |
| | | | CACGCAAGACCCAATGCAAGC | |
| | | | GCCGTCTGGGTAACGAGCACGA | |
| | | | GGAATCCAACTCCCCGCTGGCT | |
| | | | ACTCCGTGTACAGCCGAAATCT | |
| | | | ATGCCTATGCCTATAAGAATCT | |
| | | | GAAAGTGGTCCAATGCACAGA | |
| | | | GACAGACATTAGAGAAGTGCA | |
| | | | ACAGCTCCTGCTCGGCACACTG | |
| | | | AATATCGTCTGCCCTATCTGTG | |
| | | | CCCCTAAGACAACAGCCGAAAT | |
| | | | CTATAGCTATGCCTATAAGCAA | |
| | | | CTGAAAGTGCTCCAATGCAAGC | |
| | | | GCCGTCTGGGTAACGAGCACGA | |
| | | | GGAATCCAACTCCCCGCTGGCT | |
| | | | ACTCCGTGTGTTTGGCCGACTC | |
| | | | TGGACCCGTGGACCGTGGAAAC | |
| | | | TACCACTTCTTCCCTGACTATCA | |
| | | | CTACCTCCACCAAGGACGGCAC | |
| | | | CACTGTTACTGTTCAACTGCGT | |
| | | | ACCGCTGAGATTTACGCTTACG | |
| | | | CTTACAAAAACCTCAAGGTCGT | |
| | | | GATGCACGGCAGGCACGTCACC | |
| | | | CTCAAGGATATCGTCCTGGATC | |
| | | | TGCAACCCCCTGACCCTGTGGG | |
| | | | ACTGCATTGCTATGAGCAACTG | |
| | | | GTGGATTCCAGCGAAGACGAA | |
| | | | GTGGATGAGGTGGACGGACAG | |
| | | | GATAGCCAACCCCTCAAGCAAC | |
| | | | ACTTTCAGATTGTGACATGCTG | |
| | | | TCACTGTTACGAACAGCTGGAG | |
| | | | GATAGCTCCGAGGATGAGGTG | |
| | | | GACATGGAAAGCGCTAACGCC | |
| | | | AGCACAAGCGCTACCACAATCG | |
| | | | ACCAGCTCTGCAAAACCTTTAA | |
| | | | CCTCTCCATGCACACACTGCAA | |
| | | | ATCAACTGCGTCTTCTGTAAGA | |
| | | | ATGCCCTCACCACAGCCGAAAT | |
| | | | CTATAGCTATGCCTATAAGCAT | |
| | | | CTGAAAGTGCTCTTCAGGGGCG | |
| | | | GATACCCTTACGCTGCCTGTGC | |
| | | | CTGTTGCCTGGAGTTTCACGGA | |
| | | | AAGATTAACCAATACGCTCACT | |
| | | | TTGACTATGCCGGATACGCTAC | |
| | | | CACAGTGGAAGAGGAAACCAA | |
| | | | ACAGGATATCCTCGACGTGCTG | |
| | | | ATTAGATGTTACCTCTGCCATA | |
| | | | AGCCTCAGTGTGAGGTCGAGAA | |
| | | | AGTGAAACACATTCTGACAAAG | |
| | | | GCTAGATTTATCAAACTGAATT | |
| | | | GCACAAGAAAAGGCAGGTGCC | |
| | | | TCCACTGTTGGACAACCTGTAT | |
| | | | GGAAGACATGCTGCCTTGA | |
| 3′UTR | MOD- | 49 | GCGGCCGCTCTCGAGTCTAGCT | 103 |
| | 1755853 | | AGTCTAGACTAGCTAGAAAGAT | |
| | | | CCGGG | |
| SV40 poly A | MOD- | 132 | AACTTGTTTATTGCAGCTTATA | 104 |
| | 6581 | | ATGGTTACAAATAAAGCAATAG | |
| | | | CATCACAAATTTCACAAATAAA | |
| | | | GCATTTTTTTCACTGCATTCTAG | |
| | | | TTGTGGTTTGTCCAAACTCATC | |
| | | | AATGTATCTTATCATGTCTGG | |

CMV-Cytomegalovirus, TetO-tetracycline operator, TSS-transcription start site, UTR-untranslated region, MCS-multiple cloning site

Description of AdV-HPV6/11 antigen regions

| Antigen | Size | | SEQ ID |
|---|---|---|---|
| region | (aa) | Amino acid sequence | NO. |
| HPV E2 | 368 | MEAIAKRLDACAEQLLELAEENSTDLHKHVLHWKCMRH | 105 |
| | | ASVLLYKAKQMGLSHIGMQVVPPLKVSEAKGHNAIEMQ | |
| | | MHLESLLRTEYSMEPWTLQETSYAMWQTPPKRCFKKRG | |
| | | KTVEVKFDGCANNTMDYVVWTDVYVQDNDTWVKVHS | |
| | | MVDAKGIYYTCGQFKTYYVNFVKEAEKYGSTKHWEVC | |
| | | YGSTVICSPASVSSTTQEVSIPESTTYTPAQTSTLVSSSTKE | |
| | | DAVQTPPRKRARGVQQSPCNALCVAHIGPVDSGNHNLIT | |
| | | NNHDQHQRRNNSNSSATPIVQFQGESNCLKCFRYRLNDR | |
| | | HRHLFDLISSTWHWASSKAPHKHAIVTVTYDSEEQRQQF | |
| | | LDVVKIPPTISHKLGFMSLHLL | |
| HPV11 E7 | 15 | HCYEQLEDSSEDEVD | 106 |
| HPV E4 | 56 | MADDSALHKKYPFLNLLHTPPHRPPPLCPQAPRKTQCKR | 107 |
| | | RLGNEHEESNSPLATPC | |
| HPV11 E6 | 15 | TAEIYAYAYKNLKVV | 108 |
| HPV6 E7 | 29 | QCTETDIREVQQLLLGTLNIVCPICAPKT | 109 |
| HPV6 E6 | 15 | TAEIYSYAYKQLKVL | 110 |
| HPV E4 | 55 | QCKRRLGNEHEESNSPLATPCVWPTLDPWTVETTTSSLTI | 111 |
| | | TTSTKDGTTVTVQLR | |
| HPV11 E6 | 15 | TAEIYAYAYKNLKVV | 112 |
| HPV E7 | 58 | MHGRHVTLKDIVLDLQPPDPVGLHCYEQLVDSSEDEVDE | 113 |
| | | VDGQDSQPLKQHFQIVTCC | |
| HPV11 E7 | 15 | HCYEQLEDSSEDEVD | 114 |
| HPV E6 | 150 | MESANASTSATTIDQLCKTFNLSMHTLQINCVFCKNALTT | 115 |
| | | AEIYSYAYKHLKVLFRGGYPYAACACCLEFHGKINQYAH | |
| | | FDYAGYATTVEEETKQDILDVLIRCYLCHKPQCEVEKVK | |
| | | HILTKARFIKLNCTRKGRCLHCWTTCMEDMLP | |

SEQUENCE ID NO. 116: AdV-HPV6/11 antigen DNA open reading frame
sequence

ATGGAAGCCATTGCCAAAAGACTCGACGCTTGCGCCGAGCAGCTGCTGGAGCTG
GCCGAAGAGAATAGCACAGACCTCCACAAACACGTCCTGCATTGGAAGTGCATG
AGACATGCCTCCGTGCTCCTGTATAAGGCCAAGCAGATGGGACTGTCCCACATT
GGAATGCAGGTCGTGCCTCCCCTCAAGGTCAGCGAAGCCAAAGGCCATAACGCT
ATCGAAATGCAAATGCACCTGGAAAGCCTCCTGAGGACCGAATACTCCATGGAA
CCCTGGACCCTCCAGGAAACCTCCTACGCTATGTGGCAGACACCCCCTAAGAGG
TGCTTTAAGAAAAGAGGAAAGACAGTGGAAGTGAAATTCGATGGCTGTGCCAAT
AACACAATGGATTACGTCGTGTGGACCGATGTGTATGTGCAAGACAATGACACA
TGGGTCAAGGTCCACTCCATGGTGGACGCTAAGGGAATCTATTACACATGCGGA
CAGTTTAAGACATACTATGTGAATTTCGTCAAGGAAGCCGAAAAGTATGGCTCC
ACCAAACACTGGGAGGTCTGCTATGGCTCCACCGTCATCTGTAGCCCTGCCTCCG
TGTCCAGCACAACCCAAGAGGTCAGCATTCCCGAAAGCACAACCTATACCCCTG
CCCAGACCTCCACCCTCGTGTCCAGCTCCACCAAAGAGGATGCCGTCCAGACAC
CCCCTAGAAAAAGAGCTAGAGGAGTGCAACAGTCCCCCTGTAACGCTCTGTGTG
TGGCTCACATTGGCCCTGTGGATAGCGGAAACCATAACCTCATCACAAACAATC
ACGATCAGCATCAGAGGAGAAATAACTCCAACTCCAGCGCTACCCCTATCGTCC
AGTTTCAGGGAGAGTCCAACTGTCTGAAATGCTTTAGATATAGACTCAACGATA
GACATAGACATCTGTTTGACCTCATCTCCAGCACATGGCATTGGGCCAGCTCCAA
GGCTCCCCATAAGCACGCCATTGTGACAGTGACATACGATAGCGAAGAGCAAAG
ACAACAGTTTCTGGATGTGGTCAAGATTCCCCCTACCATTAGCCATAAGCTCGGC
TTTATGTCCCTGCATCTGCTCCATTGCTATGAGCAACTGGAAGACTCCAGCGAAG
ACGAAGTGGATATGGCAGACGATTCTGCACTGCATAAAAAGTACCCATTCCTGA
ACCTGCTGCATACCCCACCGCATCGCCCACCGCCACTGTGTCCGCAAGCTCCACG
CAAGACCCAATGCAAGCGCCGTCTGGGTAACGAGCACGAGGAATCCAACTCCCC
GCTGGCTACTCCGTGTACAGCCGAAATCTATGCCTATGCCTATAAGAATCTGAAA
GTGGTCCAATGCACAGAGACAGACATTAGAGAAGTGCAACAGCTCCTGCTCGGC
ACACTGAATATCGTCTGCCCTATCTGTGCCCCTAAGACAACAGCCGAAATCTATA
GCTATGCCTATAAGCAACTGAAAGTGCTCCAATGCAAGCGCCGTCTGGGTAACG
AGCACGAGGAATCCAACTCCCCGCTGGCTACTCCGTGTGTTTGGCCGACTCTGGA
CCCGTGGACCGTGGAAACTACCACTTCTTCCCTGACTATCACTACCTCCACCAAG
GACGGCACCACTGTTACTGTTCAACTGCGTACCGCTGAGATTTACGCTTACGCTT
ACAAAAACCTCAAGGTCGTGATGCACGGCAGGCACGTCACCCTCAAGGATATCG
TCCTGGATCTGCAACCCCCTGACCCTGTGGGACTGCATTGCTATGAGCAACTGGT
GGATTCCAGCGAAGACGAAGTGGATGAGGTGGACGGACAGGATAGCCAACCCC
TCAAGCAACACTTTCAGATTGTGACATGCTGTCACTGTTACGAACAGCTGGAGG
ATAGCTCCGAGGATGAGGTGGACATGGAAAGCGCTAACGCCAGCACAAGCGCT
ACCACAATCGACCAGCTCTGCAAAACCTTTAACCTCTCCATGCACACACTGCAA
ATCAACTGCGTCTTCTGTAAGAATGCCCTCACCACAGCCGAAATCTATAGCTATG
CCTATAAGCATCTGAAAGTGCTCTTCAGGGGCGGATACCCTTACGCTGCCTGTGC
CTGTTGCCTGGAGTTTCACGGAAAGATTAACCAATACGCTCACTTTGACTATGCC
GGATACGCTACCACAGTGGAAGAGGAAACCAAACAGGATATCCTCGACGTGCTG
ATTAGATGTTACCTCTGCCATAAGCCTCAGTGTGAGGTCGAGAAAGTGAAACAC
ATTCTGACAAAGGCTAGATTTATCAAACTGAATTGCACAAGAAAAGGCAGGTGC
CTCCACTGTTGGACAACCTGTATGGAAGACATGCTGCCT

SEQUENCE ID NO. 117:
AdV-HPV6/11 antigen protein sequence
M E A I A K R L D A C A E Q L L E L
A E E N S T D L H K H V L H W K C M
R H A S V L L Y K A K Q M G L S H I
G M Q V V P P L K V S E A K G H N A
I E M Q M H L E S L L R T E Y S M E
P W T L Q E T S Y A M W Q T P P K R
C F K K R G K T V E V K F D G C A N
N T M D Y V V W T D V Y V Q D N D T
W V K V H S M V D A K G I Y Y T C G
Q F K T Y Y V N F V K E A E K Y G S
T K H W E V C Y G S T V I C S P A S
V S S T T Q E V S I P E S T T Y T P
A Q T S T L V S S S T K E D A V Q T
P P R K R A R G V Q Q S P C N A L C
V A H I G P V D S G N H N L I T N N
H D Q H Q R R N N S N S S A T P I V
Q F Q G E S N C L K C F R Y R L N D
R H R H L F D L I S S T W H W A S S
K A P H K H A I V T V T Y D S E E Q
R Q Q F L D V V K I P P T I S H K L
G F M S L H L L H C Y E Q L E D S S
E D E V D M A D D S A L H K K Y P F
L N L L H T P P H R P P P L C P Q A
P R K T Q C K R R L G N E H E E S N
S P L A T P C T A E I Y A Y A Y K N
L K V V Q C T E T D I R E V Q Q L L
L G T L N I V C P I C A P K T T A E
I Y S Y A Y K Q L K V L Q C K R R L
G N E H E E S N S P L A T P C V W P
T L D P W T V E T T T S S L T I T T
S T K D G T T V T V Q L R T A E I Y
A Y A Y K N L K V V M H G R H V T L
K D I V L D L Q P P D P V G L H C Y
E Q L V D S S E D E V D E V D G Q D
S Q P L K Q H F Q I V T C C H C Y E
Q L E D S S E D E V D M E S A N A S
T S A T T I D Q L C K T F N L S M H
T L Q I N C V F C K N A L T T A E I
Y S Y A Y K H L K V L F R G G Y P Y
A A C A C C L E F H G K I N Q Y A H
F D Y A G Y A T T V E E E T K Q D I
L D V L I R C Y L C H K P Q C E V E
K V K H I L T K A R F I K L N C T R
K G R C L H C W T T C M E D M L P

SEQUENCE ID NO. 118:
AdV-HPV6/11 DNA sequence
CATCATCAATAATATACCTTATTTTGGATTGTGGCCAATATGATAA
TGAGGTGGGCGGGGAGAGGCGGGGGGGGTGACGTAGGACGCGCGA
GTAGGGTTGGGAGGTGTGGCGGAAGTGTGGCATTTGCAAGTGGGA
GGAGCTCACATGCAAGCTTCCGTCGCGGAAAATGTGACGTTTTTG
ATGAGCGCCGCCTACCTCCGGAAGTGCCAATTTTCGCGCGCTTTT
CACCGGATATCGTAGTAATTTTGGGCGGGACCATGTAAGATTTGG
CCATTTTCGCGCGAAAAGTGAAACGGGGAAGTGAAAACTGAATAA
TAGGGCGTTAGTCATAGTGCGTAATATTTACCGAGGGCCGAGGGA
CTTTGACCGATTACGTGGAGGACTCGCCCAGGTGTTTTTTACGTG
AATTTCCGCGTTCCGGGTCAAAGTCTCCGTTTTATTGTCACCGTC
ATTTGACAGATCGGGATCGGTGATCACCGATCCAGACATGATAAG
ATACATTGATGAGTTTGGACAAACCACAACTAGAATGCAGTGAAA
AAAATGCTTTATTTGTGAAATTTGTGATGCTATTGCTTTATTTGT
AACCATTATAAGCTGCAATAAACAAGTTCCCGGATCTTTCTAGCT
AGTCTAGACTAGCTAGACTCGAGAGCGGCCGCTCAAGGCAGCATG
TCTTCCATACAGGTTGTCCAACAGTGGAGGCACCTGCCTTTTCTT
GTGCAATTCAGTTTGATAAATCTAGCCTTTGTCAGAATGTGTTTC
ACTTTCTCGACCTCACACTGAGGCTTATGGCAGAGGTAACATCTA
ATCAGCACGTCGAGGATATCCTGTTTGGTTTCCTCTTCCACTGTG
GTAGCGTATCCGGCATAGTCAAAGTGAGCGTATTGGTTAATCTTT
CCGTGAAACTCCAGGCAACAGGCACAGGCAGCGTAAGGGTATCCG
CCCCTGAAGAGCACTTTCAGATGCTTATAGGCATAGCTATAGATT
TCGGCTGTGGTGAGGGCATTCTTACAGAAGACGCAGTTGATTTGC
AGTGTGTGCATGGAGAGGTTAAAGGTTTTGCAGAGCTGGTCGATT
GTGGTAGCGCTTGTGCTGGCGTTAGCGCTTTCCATGTCCACCTCA
TCCTCGGAGCTATCCTCCAGCTGTTCGTAACAGTGACAGCATGTC
ACAATCTGAAAGTGTTGCTTGAGGGGTTGGCTATCCTGTCCGTCC
ACCTCATCCACTTCGTCTTCGCTGGAATCCACCAGTTGCTCATAG
CAATGCAGTCCCACAGGGTCAGGGGGTTGCAGATCCAGGACGATA
TCCTTGAGGGTGACGTGCCTGCCGTGCATCACGACCTTGAGGTTT
TTGTAAGCGTAAGCGTAAATCTCAGCGGTACGCAGTTGAACAGTA
ACAGTGGTGCCGTCCTTGGTGGAGGTAGTGATAGTCAGGGAAGAA
GTGGTAGTTTCCACGGTCCACGGGTCCAGAGTCGGCCAAACACAC
GGAGTAGCCAGCGGGGAGTTGGATTCCTCGTGCTCGTTACCCAGA
CGGCGCTTGCATTGGAGCACTTTCAGTTGCTTATAGGCATAGCTA
TAGATTTCGGCTGTTGTCTTAGGGGCACAGATAGGGCAGACGATA
TTCAGTGTGCCGAGCAGGAGCTGTTGCACTTCTCTAATGTCTGTC
TCTGTGCATTGGACCACTTTCAGATTCTTATAGGCATAGGCATAG
ATTTCGGCTGTACACGGAGTAGCCAGCGGGGAGTTGGATTCCTCG
TGCTCGTTACCCAGACGGCGCTTGCATTGGGTCTTGCGTGGAGCT
TGCGGACACAGTGGCGGTGGGCGATGCGGTGGGGTATGCAGCAGG
TTCAGGAATGGGTACTTTTTATGCAGTGCAGAATCGTCTGCCATA
TCCACTTCGTCTTCGCTGGAGTCTTCCAGTTGCTCATAGCAATGG
AGCAGATGCAGGGACATAAAGCCGAGCTTATGGCTAATGGTAGGG
GGAATCTTGACCACATCCAGAAACTGTTGTCTTTGCTCTTCGCTA
TCGTATGTCACTGTCACAATGGCGTGCTTATGGGGAGCCTTGGAG
CTGGCCCAATGCCATGTGCTGGAGATGAGGTCAAACAGATGTCTA
TGTCTATCGTTGAGTCTATATCTAAAGCATTTCAGACAGTTGGAC
TCTCCCTGAAACTGGACGATAGGGGTAGCGCTGGAGTTGGAGTTA
TTTCTCCTCTGATGCTGATCGTGATTGTTTGTGATGAGGTTATGG
TTTCCGCTATCCACAGGGCCAATGTGAGCCACACACAGAGCGTTA
CAGGGGGACTGTTGCACTCCTCTAGCTCTTTTTCTAGGGGGTGTC
TGGACGGCATCCTCTTTGGTGGAGCTGGACACGAGGGTGGAGGTC
TGGGCAGGGGTATAGGTTGTGCTTTCGGGAATGCTGACCTCTTGG
GTTGTGCTGGACACGGAGGCAGGGCTACAGATGACGGTGGAGCCA
TAGCAGACCTCCCAGTGTTTGGTGGAGCCATACTTTTCGGCTTCC
TTGACGAAATTCACATAGTATGTCTTAAACTGTCCGCATGTGTAA
TAGATTCCCTTAGCGTCCACCATGGAGTGGACCTTGACCCATGTG
TCATTGTCTTGCACATACACATCGGTCCACACGACGTAATCCATT
GTGTTATTGGCACAGCCATCGAATTTCACTTCCACTGTCTTTCCT
CTTTTCTTAAAGCACCTCTTAGGGGGTGTCTGCCACATAGCGTAG
GAGGTTTCCTGGAGGGTCCAGGGTTCCATGGAGTATTCGGTCCTC
AGGAGGCTTTCCAGGTGCATTTGCATTTCGATAGCGTTATGGCCT
TTGGCTTCGCTGACCTTGAGGGGAGGCACGACCTGCATTCCAATG
TGGGACAGTCCCATCTGCTTGGCCTTATACAGGAGCACGGAGGCA
TGTCTCATGCACTTCCAATGCAGGACGTGTTTGTGGAGGTCTGTG
CTATTCTCTTCGGCCAGCTCCAGCAGCTGCTCGGCGCAAGCGTCG
AGTCTTTTGGCAATGGCTTCCATGGTGGCGGGTACCGGCTGCAGT
TGGACCTGGGAGTGGACACCTGTGGAGAGAAAGGCAAAGTGGATG
TCATTGTCACTCAAGTGTATGGCCAGATCTCAAGCCTGCCACACC
TCAAGTGAAGCCAAGGGGGTGGGCCTATAGACTCTATAGGCGGTA
CTTACGTCACTCTTGGCACGGGGAATCCGCGTTCCAATGCACCGT
TCCCGGCCGCGGAGGCTGGATCGGTCCCGGTGTCTTCTATGGAGG
TCAAAACAGCGTGGATGGCGTCTCCAGGCGATCTGACGGTTCACT
AAACGAGCTCGTCGACGATCTCTATCACTGATAGGGAGATCTCTA
TCACTGATAGGGAGAGCTCTGCTTATATAGACCTCCCACCGTACA
CGCCTACCGCCCATTTGCGTCAATGGGGCGGAGTTGTTACGACAT
TTTGGAAAGTCCCGTTGATTTTGGTGCCAAAACAAACTCCCATTG
ACGTCAATGGGGTGGAGACTTGGAAATCCCCGTGAGTCAAACCGC
TATCCACGCCCATTGATGTACTGCCAAAACCGCATCACCATGGTA
ATAGCGATGACTAATACGTAGATGTACTGCCAAGTAGGAAAGTCC
CATAAGGTCATGTACTGGGCATAATGCCAGGCGGGCCATTTACCG
TCATTGACGTCAATAGGGGGCGTACTTGGCATATGATACACTTGA
TGTACTGCCAAGTGGGCAGTTTACCGTAAATACTCCACCCATTGA
CGTCAATGGAAAGTCCCTATTGGCGTTACTATGGGAACATACGTC
ATTATTGACGTCAATGGGCGGGGGTCGTTGGGCGGTCAGCCAGGC
GGGCCATTTACCGTAAGTTATGTAACGCGGAACTCCATATATGGG
CTATGAACTAATGACCCCGTAATTGATTACTATTAATAACTAGAG
GCCTGACCATCTGGTGCTGGCCTGCACCAGGGCCGAGTTTGGGTC
TAGCGATGAGGATACCGATTGAGGTGGGTAAGGTGGGCGTGGCTA
GAAGGGTGGGGCGTGTATAAATTGGGGGTCTAAGGGTCTCTCTGT
TTTGTCTTGCAACAGCCGCCGCCATGAGCGACACCGGCAACAGCT
TTGATGGAAGCATCTTTAGCCCCTATCTGACAGTGCGCATGCCTC
ACTGGGCTGGAGTGCGTCAGAATGTGATGGGTTCCAACGTGGATG
GACGCCCCGTTCTGCCTTCAAATTCGTCTACAATGGCCTACGCGA
CCGTGGGAGGAACTCCGCTGGACGCCGCGACCTCCGCCGCCGCCT
CCGCCGCCGCCGCGACCGCGCGCAGCATGGCTACGGACCTTTACA
GCTCTTTGGTGGCGAGCGGCGCGGCCTCTCGCGCGTCTGCTCGGG
ATGAGAAACTGACCGCTCTGCTGCTTAAACTGGAAGACTTGACCC
GGGAGCTGGCTCAACTGACCCAGCAGGTCTCCAGCTTGCGTGAGA
GCAGCCTTGCCTCCCCCTAATGGCCCATAATATAAATAAAAGCCA
GTCTGTTTGGATTAAGCAAGTGTATGTTCTTTATTTAACTCTCCG
CGCGCGGTAAGCCCGGGACCAGCGGTCTCGGTCGTTTAGGGTGCG
GTGGATTCTTTCCAACACGTGGTACAGGTGGCTCTGGATGTTTAG
ATACATGGGCATGAGTCCATCCCTGGGGTGGAGGTAGCACCACTG
CAGAGCTTCGTGCTCGGGGGTGGTGTTGTATATGATCCAGTCGTA
GCAGGAGCGCTGGGCGTGGTGCTGAAAAATGTCCTTAAGCAAGAG
GCTTATAGCTAGGGGGAGGCCCTTGGTGTAAGTGTTTACAAATCT
GCTCAGTTGGGAGGGGTGCATCCGGGGGGATATAATGTGCATCTT
GGACTGGATTTTTAGGTTGGCTATGTTCCCACCCAGATCCCTTCT
GGGATTCATGTTGTGCAGGACCACCAGCACGGTATATCCAGTACA
CTTGGGAAATTTATCGTGGAGCTTAGACGGGAATGCATGGAAGAA
CTTGGAGACGCCCTTGTGGCCTCCCAGATTTTCCATACATTCGTC
CATGATGATGGCAATGGGCCCGTGGGAAGCTGCCTGAGCAAAAAT
GTTTCTGGGATCGCTCACATCGTAGTTATGTTCCAGGGTGAGGTC
ATCATAGGACATCTTTACAAATCGGGGGCGGAGGGTCCCGGACTG
GGGGATGATGGTGCCCTCGGGCCCCGGGGCGTAGTTCCCCTCACA
GATCTGCATCTCCCAGGCTTTCATTTCAGAGGGAGGGATCATATC
CACCTGCGGAGCGATGAAAAACACAGTTTCTGGCGCAGGGGAGAT
TAACTGGGATGAGAGCAGGTTTCTGAGCAGCTGTGACTTTCCACA
GCCGGTGGGCCCATATATCACGCCTATCACCGGCTGCAGCTGGTA
GTTAAGAGAGCTGCAGCTGCCGTCCTCCCGGAGCAGGGGGGCCAC
CTCGTTCAGCATATCCCTGACGTGGATGTTCTCCCTGACCAATTC
CGCCAGAAGGCGCTCGCCGCCCAGCGAAAGCAGCTCTTGCAAGGA
AGCAAAATTTTTCAGCGGTTTTAGGCCGTCGGCCGTGGGCATGTT
TTTCAGCGTCTGGGTCAGCAGTTCCAGTCTGTCCCACAGCTCGGT
GATGTGCTCTACGGCATCTCGATCCAGCAGATCTCCTCGTTTCGC
GGGTTGGGGCGGCTTTCGCTGTAGGGCACCAGCCGATGGGCGTCC
AGCGGGGCCAGAGTCATGTCCTTCCATGGGCGCAGGGTCCTCGTC
AGGGTGGTCTGGGTCACGGTGAAGGGGTGCGCTCCGGGTTGGGCG
CTGGCCAGGGTGCGCTTGAGGCTGGTTCTGCTGGTGCTGAATCGC
TGCCGCTCTTCGCCCTGCGCGTCGGCCAGGTAGCATTTGACCATG
GTCTCGTAGTCGAGACCCTCGGCGGCGTGCCCCTTGGCGCGGAGC
TTTCCCTTGGAGGTGGCGCCGCACGAGGGGCACTGCAGGCTCTTC
AGGGCGTAGAGCTTGGGAGCGAGAAACACGGACTCTGGGGAGTAG
GCGTCCGCGCCGCAGGAAGCGCAGACCGTCTCGCATTCCACCAGC
CAAGTGAGCTCCGGGCGGTCAGGGTCAAAAACCAGGTTGCCCCCA
TGCTTTTTGATGCGTTTCTTACCTCGGCTCTCCATGAGGCGGTGT
CCCTTCTCGGTGACGAAGAGGCTGTCCGTGTCTCCGTAGACCGAC
TTCAGGGGCCTGTCTTCCAGCGGAGTGCCTCTGTCCTCCTCGTAG
AGAAACTCTGACCACTCTGAGACGAAGGCCCGCGTCCAGGCCAGG
ACGAAGGAGGCCACGTGGGAGGGGTAGCGGTCGTTGTCCACTAGC
GGGTCCACCTTCTCCAGGGTGTGCAGGCACATGTCCCCCTCCTCC
GCGTCCAGAAAAGTGATTGGCTTGTAGGTGTAGGACACGTGACCG
GGGGTTCCCGACGGGGGGGTATAAAAGGGGGTGGGCACCCTTTCA
TCTTCACTCTCTTCCGCATCGCTGTCTGCGAGAGCCAGCTGCTGG
GGTAAGTATTCCCTCTCGAAGGCGGGCATGACCTCAGCGCTCAGG
TTGTCAGTTTCTAAAAATGAGGAGGATTTGATGTTCACCTGTCCG
GAGGTGATACCTTTGAGGGTACCTGGGTCCATCTGGTCAGAAAAC
ACTATTTTTTTGTTGTCAAGCTTGGTGGCGAACGACCCGTAGAGG
GCGTTGGAGAGCAGCTTGGCGATGGAGCGCAGGGTCTGGTTTTTG
TCGCGGTCGGCTCGCTCCTTGGCCGCGATGTTGAGTTGCACGTAC
TCGCGGGCCACGCACTTCCACTCGGGGAAGACGGTGGTGCGCTCG
TCTGGGATCAGGCGCACCCTCCAGCCTCGGTTGTGCAGGGTGACC
ATGTCGACGCTGGTGGCGACCTCGCCGCGCAGGCGCTCGTTGGTC
CAGCAGAGGCGGCCGCCCTTGCGCGAGCAGAAGGGGGGTAGGGGG
TCCAGCTGGTCCTCGTTTGGGGGGTCCGCGTCGATGGTGAAGACC
CCGGGGAGCAAGCGCGGGTCAAAGTAGTCGATCTTGCAAGCTTGC
ATGTCCAGAGCCCGCTGCCATTCGCGGGCGGCGAGCGCGCGCTCG
TAGGGGTTGAGGGGGGGGCCCCAGGGCATGGGGTGGGTGAGCGCG
GAGGCGTACATGCCGCAGATGTCATACACGTACAGGGGTTCCCTG
AGGATGCCGAGGTAGGTGGGGTAGCAGCGCCCCCCGCGGATGCTG
GCGCGCACGTAGTCATAGAGCTCGTGGGAGGGGGCCAGCATGTTG
GGCCCGAGGTTGGTGCGCTGGGGGCGCTCGGCGCGGAAGGCGATC
TGCCTGAAGATGGCATGGGAGTTGGAGGAGATGGTGGGCCGCTGG
AAGACGTTGAAGCTTGCTTCTTGCAAGCCCACCGAGTCCCTGACG
AAGCAGGCGTAGGACTCGCGCAGCTTGTGCACCAGCTCGGCGGTG
ACCTGGACGTCGAGCGCGCAGTAGTCGAGGGTCTCGCGGATGATG
TCATACTTATCCTCCCCCTTCTTTTTCCACAGCTCGCGGTTGAGG
ACGAACTCTTCGCGGTCTTTCCAGTACTCTTGGAGGGGAAACCCG
TCCGTGTCCGAACGGTAAGAGCCTAGCATGTAGAACTGGTTGACG
GCCTGGTAGGGGCAACAGCCCTTCTCCACGGGCAGCGCGTAGGCC
TGCGCCGCCTTGCGGAGGGAGGTGTGGGTGAGGGCGAAAGTGTCC
CTGACCATGACTTTGAGGTATTGATGTTTGAAGTCTGTGTCATCG
CAGCCGCCCTGTTCCCACAGGGTGTAGTCCGTGCGCTTTTTGGAG
CGCGGGTTGGGCAGGGAGAAGGTGAGGTCATTGAAGAGGATCTTC
CCCGCTCGAGGCATGAAGTTTCTGGTGATGCGAAAGGGCCCTGGG
ACCGAGGAGCGGTTGTTGATGACCTGGGCGGCCAGGACGATCTCG
TCAAAGCCGTTTATGTTGTGGCCCACGATGTAGAGCTCCAAAAAG
CGGGGCTGGCCCTTGATGGAGGGGAGCTTTTTGAGTTCCTCGTAG
GTGAGCTCCTCGGGCGATTCCAGGCCGTGCTCCTCCAGGGCCCAG
TCTTGCAAGTGAGGGTTGGCCGCCAGGAAGGATCGCCAGAGGTCG
CGGGCCATGAGGGTCTGCAGGCGGTCGCGGAAGGTTCTGAACTGT
CGCCCCACGGCCATCTTTTCGGGGGTGATGCAGTAGAAGGTGAGG
GGGTCTTTCTCCCAGGGGTCCCATCTGAGCTCTCGGGCGAGGTCG
CGCGCGGCGGCGACCAGAGCCTCGTTGCCCCCCAGTTTCATGACC
AGCATGAAGGGCACGAGCTGCTTGCCAAAGGCTCCCATCCAAGTG
TAGGTCTCTACATCGTAGGTGACAAAGAGGCGCTCCGTGCGAGGA
TGAGAGCCGATCGGGAAGAACTGGATCTCCCGCCACCAGTTGGAG
GATTGGCTGTTGATGTGGTGAAAGTAGAAGTCCCGTCTGCGGGCC
GAGCACTCGTGCTGGCTTTTGTAAAAGCGACCGCAGTACTGGCAG
CGCTGCACGGGTTGTATATCTTGCACGAGGTGAACCTGGCGACCT
CTGACGAGGAAGCGCAGCGGGAATCTAAGTCCCCCGCCTGGGGTC
CCGTGTGGCTGGTGGTCTTCTACTTTGGTTGTCTGGCCGCCAGCA
TCTGTCTCCTGGAGGGCGATGGTGGAGCAGACCACCACGCCGCGA
GAGCCGCAGGTCCAGATCTCGGCGCTCGGCGGGCGGAGTTTGATG
ACGACATCGCGCACATTGGAGCTGTCCATGGTCTCCAGCTCCCGC
GGCGGCAGGTCAGCTGGGAGTTCCTGGAGGTTCACCTCGCAGAGA
CGGGTCAAGGCGCGGGCAGTGTTGAGATGGTATCTGATTTCAAGG
GGCGTGTTGGCGGCGGAGTCGATGGCTTGCAGGAGGCCGCAGCCC
CGGGGGGCCACGATGGTTCCCCGCGGGGCGCGAGGGGAGGCGGAA
GCTGGGGGTGTGTTCAGAAGCGGTGACGCGGGCGGGCCCCCGGAG
GTAGGGGGGGTTCCGGCCCCACAGGCATGGGCGGCAGGGGCACGT
CTTCGCCGCGCGCGGGCAGGGGCTGGTGCTGGCTCCGAAGAGCGC
TTGCGTGCGCGACGACGCGACGGTTGGTGTCCTGTATCTGACGCC
TCTGAGTGAAGACCACGGGTCCCGTGACCTTGAACCTGAAAGAGA
GTTCGACAGAATCAATCTCGGCATCGTTGACAGCGGCCTGGCGCA
GGATCTCCTGCACGTCGCCCGAGTTGTCCTGGTAGGCGATCTCTG
CCATGAACTGCTCGATCTCTTCTTCCTGGAGATCTCCTCGTCCGG
CGCGCTCCACGGTGGCCGCCAGGTCGTTGGAGATGCGACCCATGA
GCTGCGAGAAGGCGTTGAGCCCGCCCTCGTTCCAGACCCGGCTGT
AGACCACGCCCCCCTCGGCGTTGCGGGCGCGCATGACCACCTGGG
CCAGGTTGAGCTCCACGTGTCGCGTGAAGACGGCGTAGTTGCGCA
GGCGCTGGAAAAGGTAGTTCAGGGTGGTGGCGGTGTGCTCGGCGA
CGAAGAAGTACATGACCCAGCGCCGCAACGTGGATTCATTGATGT
CCCCCAAGGCCTCCAGGCGCTCCATGGCCTCGTAGAAGTCCACGG
CGAAGTTGAAAAACTGGGAGTTGCGAGCGGACACGGTCAACTCCT
CCTCCAGAAGACGGATGAGCTCGGCGACAGTGTCGCGCACCTCGC
GCTCGAAGGCCACGGGGGGCGCTTCTTCCTCTTCCACCTCTTCTT
CCATGATCGCTTCTTCTTCTTCCTCAGCCGGGACGGGAGGGGGCG
GCGGCGGCGGGGGAGGGGCGCGGCGGCGGCGGCGGCGCACCGGGA
GGCGGTCGATGAAGCGCTCGATCATCTCCCCCCGCATGCGGCGCA
TGGTCTCGGTGACGGCGCGGCCGTTCTCCCGGGGGCGCAGCTCGA
AGACGCCGCCTCTCATCTCGCCGCGGGGGGGGCGGCCGTGAGGTA
GCGAGACGGCGCTGACTATGCATCTTAACAATTGCTGTGTAGGTA
CACCGCCGAGGGACCTGATTGAGTCCAGATCCACCGGATCCGAAA
ACCTTTGGAGGAAAGCGTCTATCCAGTCGCAGTCGCAAGGTAGGC
TGAGCACCGTGGCGGGCGGGGGCGGGTCTGGAGAGTTCCTGGCGG
AGATGCTGCTGATGATGTAATTAAAGTAGGCGGTCTTGAGAAGGC
GGATGGTGGACAGGAGCACCATGTCTTTGGGTCCGGCCTGTTGGA
TGCGGAGGCGGTCGGCCATGCCCCAGGCCTCGTTCTGACACCGGC
GCAGGTCTTTGTAGTAGTCTTGCATGAGTCTTTCCACCGGCACCT
CTTCTCCTTCCTCTTCTCCATCTCGCCGGTGGTTTCTCGCGCCGC
CCATGCGCGTGACCCCAAAGCCCCTGAGCGGCTGCAGCAGGGCCA
GGTCGGCGACCACGCGCTCGGCCAAGATGGCCTGCTGCACCTGAG
TGAGGGTCCTCTCGAAGTCATCCATGTCCACGAAGCGGTGGTAGG
CGCCCGTGTTGATGGTGTAGGTGCAGTTGGCCATGACGGACCAGT
TGACGGTCTGGTGTCCCGGCTGCGAGAGCTCCGTGTACCGCAGGC
GCGAGAAGGCGCGGGAATCGAACACGTAGTCGTTGCAAGTCCGCA
CCAGATACTGGTAGCCCACCAGGAAGTGCGGCGGAGGTTGGCGAT
AGAGGGGCCAGCGCTGGGTGGCGGGGGCGCCGGGCGCCAGGTTTT
CCAGCATGAGGCGGTGGTATCCGTAGATGTACCTGGACATCCAGG
TGATGCCGGCGGCGGTGGTGGTGGCGCGCGCGTAGTCGCGGACCC
GGTTCCAGATGTTTCGCAGGGGCGAGAAGTGTTCCATGGTCGGCA
CGCTCTGGCCGGTGAGGCGCGCGCAGTCGTTGACGCTCTATACAC
ACACAAAAACGAAAGCGTTTACAGGGCTTTCGTTCTGTAGCCTGG
AGGAAAGTAAATGGGTTGGGTTGCGGTGTGCCCCGGTTCGAGACC
AAGCTGAGCTCGGCCGGCTGAAGCCGCAGCTAACGTGGTATTGGC
AGTCCCGTCTCGACCCAGGCCCTGTATCCTCCAGGATACGGTCGA
GAGCCCTTTTGCTTTCTTGGCCAAGCGCCCGTGGCGCGATCTGGG
ATAGATGGTCGCGATGAGAGGACAAAAGCGGCTCGCTTCCGTAGT
CTGGAGAAACAATCGCCAGGGTTGCGTTGCGGCGTACCCCGGTTC
GAGCCCCTATGGCGGCTTGAATCGGCCGGAACCGCGGCTAACGAG
GGCCGTGGCAGCCCCGTCCTCAGGACCCCGCCAGCCGACTTCTCC
AGTTACGGGAGCGAGCCCCTTTTGTTTTTTATTTTTTAGATGCAT
CCCGTGCTGCGGCAGATGCGCCCCTCGCCCCGGCCCGATCAGCAG
CAGCAACAGCAGGCATGCAGACCCCCCTCTCCCCTTTCCGCCCCG
GTCACCACGGCCGCGGCGGCCGTGTCGGGCGCGGGGGGCGCGCTG
GAGTCAGATGAGCCACCGCGGCGGCGACCTAGGCAGTATCTGGAC
TTGGAAGAGGGCGAGGGACTGGCGCGGCTGGGGGCGAACTCTCCA
GAGCGCCACCCGCGGGTGCAGTTGAAAAGGGACGCGCGCGAGGCG
TACCTGCCGCGGCAGAACCTGTTTCGCGACCGCGGGGGCGAGGAG
CCCGAGGAGATGCGAGACTGCAGGTTCCAAGCGGGGCGCGAGCTG
CGGCGCGGGCTGGACAGACAGCGCCTGCTGCGCGAGGAGGACTTT
GAGCCCGACACGCAGACGGGCATCAGCCCCGCGCGCGCGCACGTA
GCCGCGGCCGACCTGGTGACCGCCTACGAGCAGACGGTGAACCAG
GAGCGCAACTTCCAAAAGAGCTTCAACAACCACGTGCGCACGCTG
GTGGCGCGCGAGGAGGTGACCCTGGGTCTCATGCATCTGTGGGAC
CTGGTGGAGGCGATCGTGCAGAACCCCAGCAGCAAGCCCCTGACC
GCGCAGCTGTTCCTGGTGGTGCAGCACAGCAGGGACAACGAGGCC
TTCAGGGAGGCGCTGCTGAACATCACCGAGCCGGAGGGGCGCTGG
CTCCTGGACCTGATAAACATCCTGCAGAGCATAGTGGTGCAGGAG
CGCAGCCTGAGCCTGGCCGAGAAGGTGGCGGCCATCAACTACTCT
ATGCTGAGCCTGGGCAAGTTCTACGCCCGCAAGATCTACAAGACC
CCCTACGTGCCCATAGACAAGGAGGTGAAGATAGACAGCTTCTAC
ATGCGCATGGCGCTGAAGGTGCTGACCCTGAGCGACGACCTGGGA
GTGTACCGCAACGAGCGCATCCACAAGGCCGTGAGCGCCAGCCGG
CGGCGCGAGCTGAGCGACCGCGAGCTGATGCACAGTCTGCAGCGC
GCGCTGACCGGCGCGGGCGAGGGCGACAGGGAGGTCGAGTCCTAC
TTCGACATGGGGGCCGACCTGCACTGGCAGCCGAGCCGCCGCGCC
CTGGAGGCGGCGGGGGCGTACGGCGGCCCCCTGGCGGCCGATGAC
CAGGAAGAGGAGGACTATGAGCTAGAGGAGGGCGAGTACCTGGAG
GACTGACCTGGCTGGTGGTGTTTTGGTATAGATGCAAGATCCGAA
CGTGGCGGACCCGGCGGTCCGGGCGGCGCTGCAAAGCCAGCCGTC
CGGCATTAACTCCTCTGACGACTGGGCCGCGGCCATGGGTCGCAT
CATGGCCCTGACCGCGCGCAACCCCGAGGCTTTCAGGCAGCAGCC
TCAGGCCAACCGGCTGGCGGCCATCTTGGAAGCGGTAGTGCCCGC
GCGCTCCAACCCCACCCACGAGAAGGTGCTGGCCATAGTCAACGC
GCTGGCGGAGAGCAGGGCCATCCGCGCGGACGAGGCCGGACTGGT
GTACGATGCGCTGCTGCAGCGGGTGGCGCGGTACAACAGCGGCAA
CGTGCAGACCAACCTGGACCGCCTGGTGACGGACGTGCGCGAGGC
CGTGGCGCAGCGCGAGCGCTTGCATCAGGACGGTAACCTGGGCTC
GCTGGTGGCGCTAAACGCCTTCCTCAGCACCCAGCCGGCCAACGT
ACCGCGGGGGCAGGAGGACTACACCAACTTTTTGAGCGCGCTGCG
GCTGATGGTGACCGAGGTCCCTCAGAGCGAGGTGTACCAGTCGGG
GCCCGACTACTTCTTCCAGACCAGCAGACAGGGCTTGCAAACCGT
GAACCTGAGCCAGGCTTTCAAGAACCTGCGGGGGCTGTGGGGAGT
GAAGGCGCCCACCGGCGACCGGGCTACGGTGTCCAGCCTGCTAAC
CCCCAACTCGCGCCTGCTGCTGCTGCTGATCGCGCCCTTCACGGA
CAGCGGGAGCGTCTCGCGGGAGACCTATCTGGGCCACCTGCTGAC
GCTGTACCGCGAGGCCATCGGGCAGGCGCAGGTGGACGAGCACAC
CTTCCAAGAGATCACCAGCGTGAGCCACGCGCTGGGGCAGGAGGA
CACGGGCAGCCTGCAGGCGACCCTGAACTACCTGCTGACCAACAG
GCGGCAGAAGATTCCCACGCTGCACAGCCTGACCCAGGAGGAGGA
GCGCATCTTGCGCTACGTGCAGCAGAGCGTGAGCCTGAACCTGAT
GCGCGACGGCGTGACGCCCAGCGTGGCGCTGGACATGACCGCGCG
CAACATGGAACCGGGCATGTACGCCTCCCACCGGCCGTTCATCAA
CCGCCTGATGGACTACTTGCATCGGGCGGCGGCCGTGAACCCCGA
GTACTTCACTAATGCCATTCTGAATCCCCACTGGATGCCCCCTCC
GGGTTTCTACAACGGGGACTTTGAGGTGCCCGAGGTCAACGACGG
GTTCCTCTGGGATGACATGGATGACAGTGTGTTCTCACCCAACCC
GCTGCGCGCCGCGTCTCTGCGATTGAAGGAGGGCTCTGACAGGGA
AGGACCGAGAAGTCTGGCCTCCTCCCTGGCTCTGGGAGCGGTGGG
CGCCACGGGCGCGGCGGCGCGGGGCAGTAGCCCCTTCCCCAGCCT
GGCAGACTCTCTGAACAGCGGGCGGGTGAGCAGGCCCCGCTTGCT
AGGCGAGGAGGAGTATCTGAACAACTCCCTGCTGCAGCCCGCGAG
GGACAAGAACGCTCAGCGGCAGCAGTTTCCCAACAATGGGATAGA
GAGCCTGGTGGACAAGATGTCCAGATGGAAGACGTATGCGCAGGA
GTACAAGGAGTGGGAGGACCGCCAGCCGCGGCCCTTGCCGCCCCC
TAGGCAGCGCTGGCAGCGGCGCGCGTCCAACCGCCGCTGGAGGCA
GGGGCCCGAGGACGATGATGACTCTGCAGATGACAGCAGCGTGTT
GGACCTGGGCGGGAGCGGGAACCCCTTTTCGCACCTGCGCCCACG
CCTGGGCAAGATGTTTTAAAAGAAAAAAAAAAAAATAAAACTCAC
CAAGGCCATGGCGACGAGCGTTGGTTTTTTGTTCCCTTCCTTAGT
ATGCGGCGCGCGGCGATGTTCGAGGAGGGGCCTCCCCCCTCTTAC
GAGAGCGCGATGGGGATTTCTCCTGCGGCGCCCCTGCAGCCTCCC
TACGTGCCTCCTCGGTACCTGCAACCTACAGGGGGGAGAAATAGC
ATCTGTTACTCTGAGCTGCAGCCCCTGTACGATACCACCAGACTG
TACCTGGTGGACAACAAGTCCGCGGACGTGGCCTCCCTGAACTAC
CAGAACGACCACAGCGATTTTTTGACCACGGTGATCCAAAACAAC
GACTTCACCCCAACCGAGGCCAGCACTCAGACCATAAACCTGGAT
AACAGGTCGAACTGGGGCGGCGACCTGAAGACCATCTTGCACACC
AACATGCCCAACGTGAACGAGTTCATGTTCACCAACTCTTTTAAG
GCGCGGGTGATGGTGGCGCGCGAGCAGGGGGAGGCGAAGTACGAG
TGGGTGGACTTCACGCTGCCCGAGGGCAACTACTCAGAGACCATG
ACTCTCGACCTGATGAACAATGCGATCGTGGAACACTATCTGAAA
GTGGGCAGGCAGAACGGGGTGAAGGAAAGCGATATCGGGGTCAAG
TTTGACACCAGAAACTTCCGTCTGGGCTGGGACCCCGTGACCGGG
CTGGTCATGCCGGGGGTCTACACCAACGAGGCCTTTCATCCCGAC
ATAGTGCTTCTGCCCGGCTGTGGGGTGGACTTCACCCAGAGCCGG
CTGAGCAACCTGCTGGGCATTCGCAAGCGGCAGCCTTTCCAGGAG
GGTTTCAAGATCACCTATGAGGATCTGAAGGGGGGCAACATTCCC
GCGCTCCTTGATCTGGACGCCTACGAGGAGAGCTTGAAACCCGAG
GAGAGCGCTGGCGACAGCGGCGAGAGTGGCGAGGAGCAAGCCGGC
GGCGGTGGCGGCGCGTCGGTAGAAAACGAAAGTACGCCCGCAGTG
GCGGCGGACGCTGCGGAGGTCGAGCCGGAGGCCATGCAGCAGGAC
GCAGAGGAGGGCGCACAGGAGGGCGCGCAGAAGGACATGAACGAT
GGGGAGATCAGGGGAGACACATTCGCCACCCGGGGCGAAGAAAAA
GAGGCAGAGGCGGCGGCGGCGGCGACGGCGGAGGCCGAAACCGAG
GTTGAGGCAGAGGCAGAGCCCGAGACCGAAGTTATGGAAGACATG
AATGATGGAGAACGTAGGGGCGACACGTTCGCCACCCGGGGCGAA
GAGAAGGCGGCGGAGGCAGAAGCCGCGGCTGAGGAGGCGGCTGCG
GCTGCGGCCAAGACTGAGGCTGCGGCTAAGGCTGAGGTCGAAGCC
AATGTTGCGGTTGAGGCTCAGGCTGAGGAGGAGGCGGCGGCTGAA
GCAGTTAAGGAAAAGGCCCAGGCAGAGCAGGAAGAGAAAAAACCT
GTCATTCAACCTCTAAAAGAAGATAGCAAAAAGCGCAGTTACAAC
GTCATCGAGGGCAGCACCTTTACCCAGTACCGCAGCTGGTACCTG
GCGTACAACTACGGCGACCCGGTCAAGGGGGTGCGCTCGTGGACC
CTGCTCTGCACGCCGGACGTCACCTGCGGCTCCGAGCAGATGTAC
TGGTCGCTGCCGAACATGATGCAAGACCCGGTGACCTTCCGCTCC
ACGCGGCAGGTTAGCAACTTCCCGGTGGTGGGCGCCGAACTGCTG
CCCGTGCACTCCAAGAGTTTTTACAACGAGCAGGCCGTCTACTCC
CAGCTGATCCGCCAGGCCACCTCTCTGACCCACGTGTTCAATCGC
TTTCCCGAGAACCAGATTTTGGCGCGCCCGCCGGCCCCCACCATC
ACCACCGTGAGTGAAAACGTTCCTGCCCTCACAGATCACGGGACG
CTACCGCTGCGCAACAGCATCTCAGGAGTCCAGCGAGTGACCATT
ACTGACGCCAGACGCCGGACCTGCCCCTACGTTTACAAGGCCTTG
GGCATAGTCTCGCCGCGCGTCCTCTCCAGTCGCACTTTTTAAAAC
ACATCTACCCACACGTTCCAAAATCATGTCCGTACTCATCTCACC
CAGCAACAACACCGGCTGGGGGCTGCGCGCGCCCAGCAAGATGTT
TGGAGGGGCGAGGAAGCGCTCCGACCAGCACCCTGTGCGCGTGCG
CGGCCACTACCGCGCGCCCTGGGGAGCGCACAAGCGCGGGCGCAC
AGGGCGCACCACTGTGGACGACGTCATTGACTCCGTAGTGGAGCA
AGCGCGCCACTACACACCCGGCGCGCCGACCGCCCCCGCCGTGTC
CACCGTGGACCAGGCGATCGAAAGCGTGGTACAGGGCGCGCGGCA
CTATGCCAACCTTAAAAGTCGCCGCCGCCGCGTGGCCCGCCGCCA
TCGCCGGAGACCCCGGGCCACCGCCGCCGCGCGCCTTACTAAGGC
TCTGCTCAGGCGCGCCAGGCGAACTGGCCACCGGGCCGCCATGAG
GGCCGCACGGCGGGCTGCCGCTGCCGCAAGCGCCGTGGCCCCGCG
GGCACGAAGGCGCGCGGCCGCCGCCGCCGCCGCCGCCATTTCCAG
CTTGGCCTCGACGCGGCGCGGTAACATATACTGGGTGCGCGACTC
GGTAACCGGCACGCGGGTACCCGTGCGCTTTCGCCCCCCGCGGAA
TTAGCACAAGACAACATACACACTGAGTCTCCTGCTGTTGTGTAT
CCCAGCGGCGACCGTCAGCAGCGGCGACATGTCCAAGCGCAAAAT
TAAAGAAGAGATGCTCCAGGTCATCGCGCCGGAGATCTATGGGCC
CCCGAAGAAGGAGGAGGATGATTACAAGCCCCGCAAGCTAAAGCG
GGTCAAAAAGAAAAAGAAAGATGATGATGATGACGAGGCGGTGGA
GTTTGTCCGCCGCATGGCACCCAGGCGCCCCGTGCAGTGGAAGGG
CCGGCGCGTGCAGCGCGTTTTGCGCCCCGGCACCGCGGTGGTCTT
CACGCCCGGCGAGCGCTCCACGCGCACTTTCAAGCGGGTGTACGA
TGAGGTGTACGGCGACGAGGACCTGTTGGAGCAGGCCAACCAGCG
CTTTGGGGAGTTTGCATATGGGAAACGGCCCCGCGAGAGTCTAAA
AGAGGACCTGCTGGCGCTACCGCTGGACGAGGGCAATCCCACCCC
GAGTCTGAAGCCGGTAACCCTGCAACAGGTGCTGCCTTTGAGCGC
GCCCAGCGAGCATAAGCGAGGGTTGAAGCGCGAAGGCGGGGACCT
GGCGCCCACCGTGCAGTTGATGGTGCCCAAGCGGCAGAAGCTGGA
GGACGTGCTGGAGAAAATGAAAGTAGAGCCCGGGATCCAGCCCGA
GATCAAGGTCCGCCCCATCAAGCAGGTGGCGCCCGGCGTGGGAGT
CCAGACCGTGGACGTTAGGATTCCCACGGAGGAGATGGAAACCCA
AACCGCCACTCCCTCTTCGGCGGCCAGCGCCACCACCGGCACCGC
TTCGGTAGAGGTGCAGACGGACCCCTGGCTACCCGCCACCGCTGT
TGCCGCCGCCGCCCCCCGTTCGCGCGGGCGCAAGAGAAATTATCC
AGCGGCCAGCGCGCTCATGCCCCAGTACGCACTGCATCCATCCAT
CGCGCCCACCCCCGGCTACCGCGGGTACTCGTACCGCCCGCGCAG
ATCAGCCGGCACTCGCGGCCGCCGCCGCCGTGCGACCACAACCAG
CCGCCGCCGTCGCCGCCGCCGCCAGCCAGTGCTGACCCCCGTGTC
TGTAAGGAAGGTGGCTCGCTCGGGGAGCACGCTGGTGGTGCCCAG
AGCGCGCTACCACCCCAGCATCGTTTAAAGCCGGTCTCTGTATGG
TTCTTGCAGATATGGCCCTCACTTGTCGCCTCCGCTTCCCGGTGC
CGGGATACCGAGGAAGAACTCACCGCCGCAGAGGCATGGCGGGCA
GCGGTCTCCGCGGCGGCCGTCGCCATCGCCGGCGCGCAAAAAGCA
GGCGCATGCGCGGCGGTGTGCTGCCTCTGCTAATCCCGCTAATCG
CCGCGGCGATCGGTGCCGTACCCGGGATCGCCTCCGTGGCCCTGC
AGGCGTCCCAGAAACGTTGACTCTTGCAACCTTGCAAGCTTGCAT
TTTTTGGAGGAAAAAATAAAAAAAGTCTAGACTCTCACGCTCGCT
TGGTCCTGTGACTATTTTGTAGAAAAAAGATGGAAGACATCAACT
TTGCGTCGCTGGCCCCGCGTCACGGCTCGCGCCCGTTCATGGGAG
ACTGGACAGATATCGGCACCAGCAATATGAGCGGTGGCGCCTTCA
GCTGGGGCAGTCTGTGGAGCGGCCTTAAAAATTTTGGTTCCACCA
TTAAGAACTATGGCAACAAAGCGTGGAACAGCAGCACGGGCCAGA
TGCTGAGAGACAAGTTGAAAGAGCAGAACTTCCAGGAGAAGGTGG
CGCAGGGCCTGGCCTCTGGCATCAGCGGGGTGGTGGACATAGCTA
ACCAGGCCGTGCAGAAAAAGATAAACAGTCATCTGGACCCCCGTC
CTCAGGTGGAGGAAATGCCTCCAGCGATGGAGACGGTGTCTCCCG
AGGGCAAAGGCGAAAAGCGCCCGCGGCCCGACAGAGAAGAGACCC
TGGTGTCACACACCGAGGAGCCGCCCTCTTACGAGGAGGCAGTCA
AGGCCGGCCTGCCCACCACTCGCCCCATAGCCCCCATGGCCACCG
GTGTGGTGGGCCACAGGCAACACACTCCCGCAACACTAGATCTGC
CCCCGCCGTCCGAGCCGCCGCGCCAGCCAAAGGCGGCGACGGTGC
CCGCTCCCTCCACTTCCGCCGCCAACAGAGTGCCCCTGCGCCGCG
CCGCGAGCGGCCCCCGGGCCTCGCGAGTTAGCGGCAACTGGCAGA
GCACACTGAACAGCATCGTGGGCCTGGGAGTGAGGAGTGTGAAGC
GCCGCCGTTGCTACTGAATGAGCAAGCTAGCTAACGTGTTGTATG
TGTGTATGCGTCCTATGTCGCCGCCAGAGGAGCTGTTGAGCCGCC
GGCGCCGTCTGCACTCCAGCGAATTTCAAGATGGCGACCCCATCG
ATGATGCCTCAGTGGTCGTACATGCACATCTCGGGCCAGGACGCT
TCGGAGTACCTGAGCCCCGGGCTGGTGCAGTTCGCCCGCGCCACA
GACACCTACTTCAACATGAGTAACAAGTTCAGGAACCCCACTGTG
GCGCCCACCCACGATGTGACCACGGACCGGTCGCAGCGCCTGACG
CTGCGGTTCATCCCCGTGGATCGGGAGGACACCGCCTACTCTTAC
AAGGCGCGGTTCACGCTGGCCGTGGGCGACAACCGCGTGCTGGAC
ATGGCCTCCACTTACTTTGACATCAGGGGGGTGCTGGACAGGGGC
CCCACCTTCAAGCCCTACTCGGGTACTGCCTACAACTCCCTGGCC
CCCAAGGGCGCTCCCAATTCTTGCGAGTGGGAACAAGAGGAAAAT
CAGGTGGTCGCTGCAGATGATGAACTTGAAGATGAAGAAGCGCAA
GCTCAAGAGGACGCCCCAGCTAAAAAAATTCATGTATATGCCCAG
GCGCCTCTTGCTGGCGAAAAGATTACCAAGGATGGTTTGCAAATA
GGTACTGAAGTTGTAGGAGATACATCTAAGGACACTTTTGCAGAC
AAAACATTCCAACCCGAACCTCAGATAGGCGAGTCTCAGTGGAAC
GAGGCTGATGCCACAGTAGCAGGAGGCAGAGTCTTGAAAAAAACC
ACCCCTATGAGACCTTGCTATGGATCCTATGCCAGGCCTACAAAT
GCCAACGGGGGTCAAGGAATTATGGTTGCCAATGAACAAGGAGTG
TTGGAGTCTAAAGTGGAGATGCAATTTTTTTCTAACACTACAACC
CTTAATGCGCGGGATGGAGCTGGCAATCCCGAACCAAAGGTGGTG
TTGTACAGTGAAGATGTCCACTTGGAATCTCCTGACACTCATTTG
TCTTACAAGCCCAAAAAGGATGATGTTAATGCTAAAATTATGTTG
GGTCAGCAAGCTATGGCTAACAGGCCCAACCTCATTGCTTTTAGA
GATAATTTCATTGGACTCATGTACTACAACAGCACTGGTAACATG
GGAGTGCTGGCGGGTCAGGCCTCTCAGTTGAATGCCGTGGTGGAC
CTGCAGGATAGAAACACAGAACTGTCATATCAGCTTATGCTTGAT
TCCATTGGGGATAGATCCAGATACTTCTCCATGTGGAACCAGGCA
GTGGATAGCTATGACCCAGATGTCAGAATCATTGAAAACCATGGT
GTCGAGGACGAGCTACCCAACTACTGCTTCCCTCTGGGCGGCATA
GGAATTACTGATACTTATCAAGGGATCAAAAATACCAATGGCAAT
GGTCAGTGGACCAAAGATGATCAGTTCGCGGACCGTAATGAAATA
GGGGTGGGAAACAACTTCGCCATGGAGATCAACATCCAGGCCAAC
CTCTGGAGGAACTTCCTCTATGCGAACGTGGGGCTCTACCTGCCA
GACAAGCTCAAGTACAACCCCACCAACGTGGACATCTCTGACAAC
CCCAACACCTATGACTACATGAACAAGCGTGTGGTGGCTCCCGGC
CTGGTGGACTGCTTTGTCAATGTGGGAGCCAGGTGGTCCCTGGAC
TACATGGACAACGTCAACCCCTTCAACCACCACCGCAATGCGGGT
CTGCGCTACCGCTCCATGATCCTGGGCAACGGGCGCTACGTGCCC
TTCCACATTCAGGTGCCCCAGAAGTTCTTTGCCATCAAGAACCTC
CTCCTCCTGCCGGGCTCCTACACTTACGAGTGGAACTTCAGGAAG
GATGTCAACATGGTCCTGCAGAGCTCTCTGGGCAATGACCTTAGG
GTGGACGGGGCCAGCATCAAGTTTGACAGCGTCACCCTCTATGCT
ACCTTCTTCCCCATGGCTCACAACACCGCCTCCACGCTCGAGGCC
ATGCTGAGGAACGACACCAACGACCAGTCCTTCAATGACTACCTC
TCTGGGGCCAACATGCTCTACCCCATCCCCGCCAAGGCCACCAAC
GTGCCCATCTCCATTCCCTCTCGCAACTGGGCCGCCTTCAGAGGC
TGGGCCTTTACCCGCCTTAAGACCAAGGAAACCCCCTCCCTGGGC
TCGGGTTTTGACCCCTACTTTGTCTACTCGGGATCCATCCCCTAC
CTGGATGGCACCTTCTACCTCAACCACACTTTTAAGAAGATATCC
ATCATGTATGACTCCTCCGTCAGCTGGCCGGGCAATGACCGCCTG
CTCACCCCCAATGAGTTCGAGGTCAAGCGCGCCGTGGACGGCGAG
GGCTACAACGTGGCCCAGTGCAACATGACCAAGGACTGGTTCCTG
GTGCAGATGCTGGCCAACTACAACATAGGCTACCAGGGCTTCTAC
ATCCCAGAGAGCTACAAGGACAGGATGTACTCCTTCTTCAGAAAT
TTCCAACCCATGAGCAGGCAGGTGGTGGACGAGACCAAATACAAG
GACTATCAGGCCATTGGCATCACTCACCAGCACAACAACTCGGGA
TTCGTGGGCTACCTGGCTCCCACCATGCGCGAGGGGCAGGCCTAC
CCCGCCAACTTCCCCTACCCGTTGATAGGCAAGACCGCGGTCGAC
AGCGTCACCCAGAAAAAGTTCCTCTGCGACCGCACCCTCTGGCGC
ATCCCCTTCTCTAGCAACTTCATGTCCATGGGTGCGCTCACGGAC
CTGGGCCAGAACCTGCTCTATGCCAACTCCGCCCATGCGCTGGAC
ATGACTTTTGAGGTGGACCCCATGGACGAGCCCACCCTTCTCTAT
ATTGTGTTTGAAGTGTTCGACGTGGTCAGAGTGCACCAGCCGCAC
CGCGGTGTCATCGAGACCGTGTACCTGCGCACGCCCTTCTCGGCC
GGCAACGCCACCACCTAAGGAGACAGCGCCGCCGCCTGCATGACG
GGTTCCACCGAGCAAGAGCTCAGGGCCATCGCCAGAGACCTGGGA
TGCGGACCCTATTTTTTGGGCACCTATGACAAACGCTTCCCGGGC
TTCATCTCCCGAGACAAGCTCGCCTGCGCCATCGTCAACACGGCC
GCGCGCGAGACCGGGGGCGTGCACTGGCTGGCCTTTGGCTGGGAC
CCGCGCTCCAAAACCTGCTACCTCTTCGACCCCTTTGGCTTCTCC
GATCAGCGCCTCAGACAGATCTATGAGTTTGAGTACGAGGGGCTG
CTGCGCCGCAGCGCGCTTGCCTCCTCGCCCGACCGCTGCATCACC
CTTGAGAAGTCCACCGAGACCGTGCAGGGGCCCCACTCGGCCGCC
TGCGGTCTCTTCTGCTGCATGTTTTTGCACGCCTTTGTGCGCTGG
CCCCAGAGTCCCATGGATCGCAACCCCACCATGAACTTGCTCAAG
GGAGTGCCCAACGCCATGCTCCAGAGCCCCCAGGTCCAGCCCACC
CTGCGCCACAACCAGGAACAGCTCTACCGCTTCCTGGAGCGCCAC
TCCCCCTACTTCCGCAGTCACAGCGCGCACATCCGGGGGGCCACC
TCTTTCTGCCACTTGCAACAAAACATGCAAGACGGAAAATGATGT
ACAGCTCGCTTTTTAATAAATGTAAAGACTGTGCACTTTATTTAT
ACACGGGCTCTTTCTGGTTATTTATTCAACACCGCCGTCGCCATC
TAGAAATCGAAAGGGTTCTGCCGCGCGTCGCCGTGCGCCACGGGC
AGAGACACGTTGCGATACTGGAAGCGGCTCGCCCACTTGAACTCG
GGCACCACCATGCGGGGCAGTGGCTCCTCGGGGAAGTTCTCGCCC
CACAGGGTGCGGGTCAGCTGCAGCGCGCTCAGGAGGTCGGGAGCC
GAGATCTTGAAGTCGCAGTTGGGGCCGGAACCCTGCGCGCGCGAG
TTGCGGTACACGGGGTTGCAGCACTGGAACACCAGCAGGGCCGGA
TTACGCACGCTGGCCAGCAGGCTCTCGTCGCTGATCATGTCGCTG
TCCAGATCCTCCGCGTTGCTCAGGGCGAATGGGGTCATCTTGCAG
ACCTGCCTGCCCAGGAAAGGCGGCAGCCCGGGCTTGCCGTTGCAG
TCGCAGCGCAGGGGCATCAGCAGGTGCCCGTGGCCCGTCTGCGCC
TGCGGGTACAGCGCGCGCATGAAGGCTTCGATCTGCCTGAAAGCC
ACCTGCGTCTTGGCTCCCTCCGAAAAGAACATCCCACAGGACTTG
CTGGAGAACTGGTTCGCGGGACAGCTGGCATCGTGCAGGCAGCAG
CGCGCGTCGGTGTTGGCGATCTGCACCACGTTGCGACCCCACCGG
TTCTTCACTATCTTGGCCTTGGAAGCCTGCTCCTTCAGCGCGCGC
TGGCCGTTCTCGCTGGTCACATCCATCTCTATCACCTGCTCCTTG
TTGATCATGTTTGTCCCGTGCAGACACTTCAGGTCGCCCTCCGTC
TGGGTGCAGCGGTGCTCCCACAGCGCGCAACCGGTGGGCTCCCAA
TTTTTGTGGGTCACCCCCGCGTAGGCCTGCAGGTAGGCCTGCAAG
AAGCGCCCCATCATGGCCACAAAGGTCTTCTGGCTCGTAAAGGTC
AGCTGCAGGCCGCGATGCTCTTCGTTCAGCCAGGTCTTGCAGATG
GCGGCCAGCGCCTCGGTCTGCTCGGGCAGCATCCTAAAATTTGTC
TTCAGGTCGTTATCCACGTGGTACTTGTCCATCATGGCGCGCGCC
GCCTCCATGCCCTTCTCCCAGGCGGACACCATGGGCAGGCTTAGG
GGGTTTATCACTTCCACCGGCGAGGACACCGTACTTTCGATTTCT
TCTTCCTCCCCCTCTTCCCGGCGCGCGCCCACGCTGCTGCGCGCT
CTCACCGCCTGCACCAAGGGGTCGTCTTCAGGCAAGCGCCGCACC
GAGCGCTTGCCGCCCTTGACCTGCTTAATCAGCACCGGCGGGTTG
CTGAAGCCCACCATGGTCAGCTCCGCCTGCTCTTCTTCGTCTTCG
CTGTCTACCACTATCTCTGGGGAAGGGCTTCTCCGCTCTGCGGCG
GTGCGCTTCTTTTTTTTCTTGGGAGCAGCCGTGACGGAGTCCGCC
ACGGCGACGGAGGTCGAGGGCGTGGGGCTGGGGGTGCGCGGTACC
AGGGCCTCGTCGCCCTCGGACTCTTCCTCTGACTCCAGGCGGCGG
CGGAGACGCTTCTTTGGGGGCGCGCGCGTCAGCGGCGGCGGAGAC
GGGGACGGGGACGGGGACGGGACGCCCTCCACAGGGGGTGGTCTT
CGCGCAGACCCGCGGCCGCGCTCGGGGGTCTTTTCGAGCTGGTCT
TGGTCCCGACTGGCCATTGTATCCTCCTCCTCCTAGGCAGAGAGA
CATAAGGAGTCTATCATGCAAGTCGAGAAGGAGGAGAGCTTAACC
ACCCCCTCTGAGACCGCCGATGCGCCCGCCGTCGCCGTCGCCCCC
GCTGCCGCCGACGCGCCCGCCACACCGAGCGACACCCCCGCGGAC
CCCCCAGCCGACGCACCCCTGTTCGAGGAAGCGGCCGTGGAGCAG
GACCCGGGCTTTGTCTCGGCAGAGGAGGATTTGCGAGAGGAGGAG
GATAAGGAGAAGAAGCCCTCAGTGCCAAAAGATGATAAAGAGCAA
GACGAGCACGACGCAGATGCACACCAGGGTGAAGTCGGGCGGGGG
GACGGAGGGCATGACGGCGCCGACTACCTAGACGAAGGGAACGAC
GTGCTCTTGAAGCACCTGCATCGTCAGTGCGCCATCGTTTGCGAC
GCTCTGCAGGAGCGCAGCGAAGTGCCCCTCAGCGTGGCGGAGGTC
AGCCACGCCTACGAGCTCAGCCTCTTCTCCCCCCGGGTGCCCCCC
CGCCGCCGCGAAAACGGCACATGCGAGCCCAACCCGCGCCTCAAC
TTCTACCCCGCCTTTGTGGTGCCCGAGGTCCTGGCCACCTATCAC
ATCTTCTTTCAAAATTGCAAGATCCCCCTCTCGTGCCGCGCCAAC
CGTAGCCGCGCCGATAAGATGCTGGCCCTGCGCCAGGGCGACCAC
ATACCTGATATCGCCGCTTTGGAAGATGTGCCAAAGATCTTCGAG
GGTCTGGGTCGCAACGAGAAGCGGGCAGCAAACTCTCTGCAACAG
GAAAACAGCGAAAATGAGAGTCACACCGGGGTACTGGTGGAGCTC
GAGGGCGACAACGCCCGCCTGGCGGTGGTCAAGCGCAGCATCGAG
GTCACCCACTTTGCCTACCCCGCGCTCAACCTGCCCCCCAAAGTC
ATGAACGCGGCCATGGACGGGCTGATCATGCGCCGCGGCCGGCCC
CTCGCTCCAGATGCAAACTTGCATGAGGAGACCGAGGACGGCCAG
CCCGTGGTCAGCGACGAGCAGCTGGCGCGCTGGCTGGAGACCGCG
GACCCCGCCGAACTGGAGGAGCGGCGCAAGATGATGATGGCCGCG
GTGCTGGTCACCGTAGAGCTGGAGTGTCTGCAGCGCTTCTTCGGC
GACCCCGAGATGCAGAGAAAGGTCGAGGAGACCCTGCACTACACC
TTCCGCCAGGGCTACGTGCGCCAGGCTTGCAAGATCTCCAACGTG
GAGCTCAGCAACCTGGTGTCCTACCTGGGCATCTTGCATGAGAAC
CGCCTCGGGCAGAGCGTGCTGCACTCCACCCTGCGCGGGGAGGCG
CGCCGCGACTACGTGCGCGACTGCGTTTACCTCTTCCTCTGCTAC
ACCTGGCAGACGGCCATGGGGGTCTGGCAGCAGTGCCTGGAGGAG
CGCAACCTCAAGGAGCTGGAGAAGCTCCTGCAGCGCGCGCTCAAA
GACCTCTGGACGGGCTACAACGAGCGCTCGGTGGCCGCCGCGCTG
GCCGACCTCATCTTCCCCGAGCGCCTGCTCAAAACCCTCCAGCAG
GGGCTGCCCGACTTCACCAGCCAAAGCATGTTGCAAAACTTCAGG
AACTTTATCCTGGAGCGTTCTGGCATCCTACCCGCCACCTGCTGC
GCCCTGCCCAGCGACTTTGTCCCCCTCGTGTACCGCGAGTGCCCC
CCGCCGCTGTGGGGTCACTGCTACCTGTTCCAACTGGCCAACTAC
CTGTCCTACCACGCGGACCTCATGGAGGACTCCAGCGGCGAGGGG
CTCATGGAGTGCCACTGCCGCTGCAACCTCTGCACGCCCCACCGC
TCCCTGGTCTGCAACACCCAACTGCTCAGCGAGAGTCAGATTATC
GGTACCTTCGAGCTACAGGGTCCGTCCTCCTCAGACGAGAAGTCC
GCGGCTCCGGGGCTAAAACTCACTCCGGGGCTGTGGACTTCCGCC
TACCTGCGCAAATTTGTACCTGAAGACTACCACGCCCACGAGATC
AGGTTTTACGAAGACCAATCCCGCCCGCCCAAGGCGGAGCTGACC
GCCTGCGTCATCACCCAGGGCGAGATCCTAGGCCAATTGCAAGCC
ATCCAAAAAGCCCGCCAAGATTTTTTGCTGAGAAAGGGTCGGGGG
GTGTATCTGGACCCCCAGTCGGGTGAGGAGCTCAACCCGGTTCCC
CCGCTGCCGCCGCCGCGGGACCTTGCTTCCCAGGATAAGCATCGC
CATGGCTCCCAGAAAGAAGCAGCAGCGGCCGCCACTGCCGCCACC
CCACATGCTGGAGGAAGAGGAGGAATACTGGGACAGTCAGGCAGA
GGAGGTTTCGGACGAGGAGGAGCCGGAGACGGAGATGGAAGAGTG
GGAGGAGGACAGCTTAGACGAGGAGGCTTCCGAAGCCGAAGAGGC
AGACGCAACACCGTCACCCTCGGCCGCAGCCCCCTCGCAGGCGCC
CCCGAAGTCCGCTCCCAGCATCAGCAGCAACAGCAGCGCTATAAC
CTCCGCTCCTCCACCGCCGCGACCCACGGCCGACCGCAGACCCAA
CCGTAGATGGGACACCACCGGAACCGGGGCCGGTAAGTCCTCCGG
GAGAGGCAAGCAAGCGCAGCGCCAAGGCTACCGCTCGTGGCGCGC
TCACAAGAACGCCATAGTCGCTTGCTTGCAAGACTGCGGGGGGAA
CATCTCCTTCGCCCGCCGCTTCCTGCTCTTCCACCACGGTGTGGC
CTTCCCCCGTAACGTCCTGCATTACTACCGTCATCTCTACAGCCC
CTACTGCGGCGGCAGTGAGCCAGAGACGGTCGGCGGCGGCGGCGG
CGCCCGTTTCGGCGCCTAGGAAGACCCAGGGCAAGACTTCAGCCA
AGAAACTCGCGGCGGCCGCGGCGAACGCGGTCGCGGGGGCCCTGC
GCCTGACGGTGAACGAACCCCTGTCGACCCGCGAACTGAGAAACC
GAATCTTCCCCACTCTCTATGCCATCTTCCAGCAGAGCAGAGGGC
AGGATCAGGAACTGAAAGTAAAAAACAGGTCTCTGCGCTCCCTCA
CCCGCAGCTGTCTGTATCACAAGAGCGAAGACCAGCTTCGGCGCA
CGCTGGAGGACGCTGAGGCACTCTTCAGCAAATACTGCGCGCTCA
CTCTTAAGGACTAGCTCCGCGCCCTTCTCGAATTTAGGCGGGAAC
GCCTACGTCATCGCAGCGCCGCCGTCATGAGCAAGGACATTCCCA
CGCCATACATGTGGAGCTATCAGCCGCAGATGGGACTCGCGGCGG
GCGCCTCCCAAGACTACTCCACCCGCATGAACTGGCTCAGTGCCG
GCCCACACATGATCTCACAGGTTAATGATATCCGCACCCATCGAA
ACCAAATATTGGTGGAGCAGGCGGCAATTACCACCACGCCCCGCA
ATAATCCCAACCCCAGGGAGTGGCCCGCGTCCCTGGTGTATCAGG
AAATTCCCGGCCCCACCACCGTACTACTTCCGCGTGATTCCCAGG
CCGAAGTCCAAATGACTAACTCAGGGGCGCAGCTCGCGGGCGGCT
GTCGTCACAGGGTGCGGCCTCCTCGCCAGGGTATAACTCACCTGG
AGATCCGAGGCAGAGGTATTCAGCTCAACGACGAGTCGGTGAGCT
CCTCGCTCGGTCTAAGACCTGACGGGACCTTCCAGATAGCCGGAG
CCGGCCGATCTTCCTTCACGCCCCGCCAGGCGTACCTGACTCTGC
AGAGCTCGTCCTCGGCGCCGCGCTCGGGCGGCATCGGGACTCTCC
AGTTCGTGCAGGAGTTTGTGCCCTCGGTCTACTTCAACCCCTTCT
CGGGCTCTCCCGGTCGCTACCCGGACCAGTTCATCTCGAACTTTG
ACGCCGCGAGGGACTCGGTGGACGGCTACGACTGAATGTCGGGTG
GACCCGGTGCAGAGCAACTTCGCCTGAAGCACCTCGACCACTGCC
GCCGCCCTCAGTGCTTTGCCCGCTGTCAGACCGGTGAGTTCCAGT
ACTTTTCCCTGCCCGACTCGCACCCGGACGGCCCGGCGCACGGGG
TGCGCTTTTTCATCCCGAGTCAGGTGCGCTCTACCCTAATCAGGG
AGTTTACCGCCCGTCCCCTACTGGCGGAGTTGGAAAAGGGGCCTT
CTATCCTAACCATTGCCTGCATCTGCTCTAACCCTGGATTGCACC
AAGATCTTTGCTGTCATTTGTGTGCTGAGTATAATAAAGGCTGAG
ATCAGAATCTACTCGGGCTCCTGTCGCCATCCTGTCAACGCCACC
GTCCAAGCCCGGCCCGATCAGCCCGAGGTGAACCTCACCTGCGGT
CTGCACCGGCGCCTGAGGAAATACCTAGCTTGGTACTACAACAGC
ACTCCCTTTGTGGTTTACAACAGCTTTGACCAGGACGGGGTCTCA
CTGAGGGATAACCTCTCGAACCTGAGCTACTCCATCAGGAAGAAC
AGCACCCTCGAGCTACTTCCTCCTTACCTGCCCGGGACTTACCAG
TGTGTCACCGGTCCCTGCACCCACACCCACCTGTTGATCGTAAAC
GACTCTCTTCCGAGAACAGACCTCAATAACTCCTCTTCGCAGTTC
CCCAGAACAGGAGGTGAGCTCAGGAAACCCCGGGTAAAGAAGGGT
GGACAAGAGTTAACACTTGTGGGGTTTCTGGTGTATGTGACGCTG
GTGGTGGCTCTTTTGATTAAGGCTTTTCCTTCCATGTCTGAACTC
TCCCTCTTTTATGAACAACTCGACTAGTGCTAACGGGACCCTACC
CAACGAATCGGGATTGAATATCGGTAACCAGGTTGCAGTTTCACT
TTTGATTACCTTCATAGTCCTCTTCCTGCTAGTGCTGTCGCTTCT
GTGCCTGCGGATCGGGGGCTGCTGCATCCACGTTTATATCTGGTG
CTGGCTGTTTAGAAGGTTCGGAGACCATCGCAGGTAGAATAAACA
TGCTGCTGCTTACCCTCTTTGTCCTGGCGCTGGCCGCCAGCTGCC
AAGCCTTTTCCGAGGCTGACTTTATAGAGCCCCAGTGTAACGTGA
CTTTTAAAGCCCATGCACAGCGTTGTCATACTATAATCAAATGTG
CCACCGAACACGATGAATACCTTATCCAGTATAAAGATAAATCAC
ACAAAGTGGCACTTGTTGACATCTGGAAACCCGAAGACCCTTTGG
AATACAATGTGACCGTTTTCCAGGGTGACCTCTTCAAAATTTACA
ATTACACTTTCCCATTTGACCAGATGTGTGACTTTGTCATGTACA
TGGAAAAGCAGCACAAGCTGTGGCCTCCGACTCCCCAGGGCTGTG
TGGAAAATCCAGGCTCTTTCTGCATGATCTCTCTCTGTGTAACTG
TGCTGGCACTAATACTCACGCTTTTGTATATCAGATTTAAATCAA
GGCAAAGCTTCATCGATGAAAAGAAAATGCCTTAAACGCTTTCAC
GCTTGATTGCTAACACCGGGTTTTTATCCGCAGAATGATTGGAAT
CACCCTACTAATCACCTCCCTCCTTGCGATTGCCCATGGGTTGGA
ACGAATCGAAGCCCCTGTGGGGGCCAATGTTACCCTGGTGGGGCC
TGTCGGCAATGCTACATTAATGTGGGAAAAATATACTAAAAATCA
ATGGGTCTCTTACTGCACTAACAAAAACAGCCACAAGCCCAGAGC
CATCTGCGATGGGCAAAATCTAACCTTGATTGATGTTCAAATGCT
GGATGCGGGCTACTATTATGGGCAGCTGGGTACAATGATTAATTA
CTGGAGACCCCACAAAGATTACATGCTCCACGTAGTAAAGGGTCC
CCTTAGCAGCCCACCCACTACCACCTCTACTACCCCCACTACCAC
CACTACTCCCACCACCAGCACTGCCGCCCAGCCTCCTCATAGCAG
AACAACCACTTTTATCAATTCCAAGTCCCACTCCCCCCACATTGC
CGGCGGGCCCTCCGCCTCAGACTCCGAGACCACCGAGATCTGCTT
CTGCAAATGCTCTGACGCCTTTGCTGAGGATTTGGAAGACCACGA
GGAAGATGAGCATGACTTCGCAGATGCATGCCAGGCATCAGAGGC
AGAAGCGCTGCCGGTGGCCCTCAAACAGTATGCAGACCCCCACAC
CACCCCCAACCTTCCTCCACCTTCCCAGAAGCCAAGTTTCCTGGG
GGAAAATGAAACTCTGCCTCTCTCCATACTCGCTCTGACATCTGT
TGCTATGTTGACCGCTCTGCTGGTGCTTCTATGCTCTATATGCTA
CCTGATCTGCTGCAGAAAGAAAAAATCTCACGGCCATGCTCACCA
GCCCCTCATGCACTTCCCTTACCCTCCAGAGCTGGGCGACCACAA
ACTTTAAGTCTGCAGTAACTATCTGCCCATCCCTTGTCAGTCGAC
AGCGATGAGCCCCACTAATCTAACGGCCTCTGGACTTACAACATC
GTCTCTTAATGAGACCACCGCTCCTCAAGACCTGTACGATGGTGT
CTCCGCGCTGGTTAACCAGTGGGATCACCTGGGCATATGGTGGCT
CCTCATAGGAGCAGTGACCCTGTGCCTAATCCTGGTCTGGATCAT
CTGCTGCATCAAAAGCAGAAGACCCAGGCGGCGGCCCATCTACAG
GCCCTTTGTCATCACACCTGAAGATGATGATGACACCACTTCCAG
GCTGCAGAGGCTAAAGCAGCTACTCTTCTCTTTTACAGCATGGTA
AATTGAATCATGCCTCGCATTTTCATCTACTTGTCTCTCCTTCCA
CTTTTTCTGGGCTCTTCTACATTGGCCGCTGTGTCCCACATCGAG
GTAGACTGCCTCACGCCCTTCACAGTCTACCTGCTTTTCGGCTTT
GTCATCTGCACCTTTGTCTGCAGCGTTATCACTGTAGTGATCTGC
TTCATACAGTGCATCGACTACGTCTGCGTGCGGGTGGCTTACTTT
AGACACCACCCCCAGTATCGCAACAGGGACATAGCGGCTCTCCTA
AGACTTGTTTAAAATCATGGCCAAATTAACTGTGATTGGTCTTCT
GATCATCTGCTGCGTCCTAGCCGCGATTGGGACTCAAGCTCCTAC
CACCACCAGCGCTCCCAGAAAGAGACATGTATCCTGCAGCTTCAA
GCGTCCCTGGAATATACCCCAATGCTTTACTGATGAACCTGAAAT
CTCTTTGGCTTGGTACTTCAGCGTCACCGCCCTTCTTATCTTCTG
CAGTACGGTTATTGCCCTTGCCATCTACCCTTCCCTTGACCTGGG
CTGGAATGCTGTCAACTCTATGGAATATCCCACCTTCCCAGAACC
AGACCTGCCAGACCTGGTTGTTCTAAACGCGTTTCCTCCTCCTGC
TCCCGTTCAAAATCAGTTTCGCCCTCCGTCCCCCACGCCCACTGA
GGTCAGCTACTTTAATCTAACAGGCGGAGATGACTGAAAACCTAG
ACCTAGAAATGGACGGTCTCTGCAGCGAGCAACGCACACTAGAGA
GGCGCCGGCAAAAAGAGCTCGAGCGTCTTAAACAAGAGCTCCAAG
ACGCGGTGGCCATACACCAGTGCAAAAAAGGTGTCTTCTGTCTGG
TAAAACAGGCCACGCTCACCTATGAAAAAACAGGTGACACCCACC
GCCTAGGATACAAGCTGCCCACACAGCGCCAGAAGTTCGCCCTCA
TGATAGGCGAACAACCCATCACCGTGACCCAGCACTCCGTGGAGA
CAGAAGGCTGCATACACGCTCCCTGTAGGGGCGCTGACTGCCTCT
ACACCTTGATCAAAACCCTCTGCGGTCTCAGAGACCTCATCCCTT
TTAATTAATCATAACTGTAATCAATAAAAAATCACTTACTTGAAA
TCTGATAGCAAGCCTCTGTCCAATTTTTTCAGCAACACTTCCTTC
CCCTCCTCCCAACTCTGGTACTCTAGGCGCCTCCTAGCTGCAAAC
TTCCTCCACAGTCTGAAGGGAATGTCAGATTCCTCCTCCTGTCCC
TCCGCACCCACGATCTTCATGTTGTTGCAGATGAAACGCGCGAGA
TCGTCTGACGAGACCTTCAACCCCGTGTACCCCTACGATACCGAG
ATCGCTCCGACTTCTGTCCCTTTCCTTACCCCTCCCTTTGTGTCA
TCCGCAGGAATGCAAGAAAATCCAGCTGGGGTGCTGTCCCTGCAC
TTGTCAGAGCCCCTTACCACCCACAATGGGGCCCTGACTCTAAAA
ATGGGGGGCGGCCTGACCCTGGACAAGGAAGGGAATCTCACTTCC
CAAAACATCACCAGTGTCGATCCCCCTCTCAAAAAAAGCAAGAAC
AACATCAGCCTTCAGACCGCCGCACCCCTCGCCGTCAGCTCCGGG
GCCCTAACACTTTTTGCCACTCCCCCCCTAGCGGTCAGTGGTGAC
AACCTTACTGTGCAGTCTCAGGCCCCTCTCACTTTGGAAGACTCA
AAACTAACTCTGGCCACCAAAGGACCCCTAACTGTGTCCGAAGGC
AAACTTGTCCTAGAAACAGAGGCTCCCCTGCATGCAAGTGACAGC
AGCAGCCTGGGCCTTAGCGTTACGGCCCCACTTAGCATTAACAAT
GACAGCCTAGGACTAGACATGCAAGCGCCCATTAGCTCTCGAGAT
GGAAAACTGGCTCTAACAGTGGCGGCCCCCCTAACTGTGGTCGAG
GGTATCAATGCTTTGGCAGTAGCCACAGGTAAGGGTATTGGGCTA
AATGAAACCAACACACACCTGCAGGCAAAACTGGTCGCACCCCTA
GGCTTTGATACCAACGGCAACATTAAGCTAAGCGTTGCAGGAGGC
ATGAGGCTAAACAATAACACACTGATACTAGATGTAAACTACCCA
TTTGAGGCTCAAGGCCAACTGAGCCTAAGAGTGGGCTCGGGCCCA
CTATATGTAGATTCTAGTAGTCATAACCTAACCATTAGATGCCTT
AGGGGATTGTATATAACATCTTCTAACAACCAAAACGGTCTAGAA
GCCAACATTAAACTAACAAGAGGCCTTGTGTATGACGGAAATGCC
ATAGCAGTTAATGTTGGCAAAGGGCTGGAATACAGCCCTACTGAC
ACAACAGAAAAACCTATACAGACTAAAATAGGTCTAGGCATGGAG
TATGATACCGAGGGAGCCATGATGACAAAACTAGGCTCTGGACTA
AGCTTTGACAATTCAGGAGCCATTGTAGTGGGAAACAAAAATGAT
GACAGGCTTACTTTGTGGACCACACCGGACCCATCGCCCAACTGT
CAGATCTACTCTGAAAAAGATGCTAAACTAACCTTGGTACTGACT
AAATGTGGCAGTCAGGTTGTAGGCACAGTATCTATTGCCGCTCTT
AAAGGTAGCCTCGTGCCAATCACTAGTGCAATCAGTGTGGTTCAG
GTATACCTAAGGTTTGATGAAAATGGGGTACTAATGAGTAACTCT
TCACTTAATGGCGAATACTGGAATTTTAGAAACGGAGACTCAACT
AATGGCACACCATATACAAACGCAGTGGGTTTCATGCCTAATCTA
CTGGCCTATCCTAAAGGTCAAACTACAACTGCAAAAAGTAACATT
GTCAGCCAGGTCTACATGAATGGGGACGATACTAAACCCATGACA
TTTACAATCAACTTCAATGGCCTTAGTGAAACAGGGGATACCCCT
GTTAGTAAATATTCCATGACATTCTCATGGAGGTGGCCAAATGGA
AGCTACATAGGGCACAATTTTGTAACAAACTCCTTTACCTTCTCC
TACATCGCCCAAGAATAAAGAAAGCACAGAGATGCTTGTTTTTGA
TTTCAAAATTGTGTGCTTTTATTTATTTTCAGCTTACAGTATTTC
CAGTAGTCATTCAAATAAAGCTTAATCAAACTGCATGAGAACCCT
TCCACATAGCTTAAATTAGCACCAGTGCAAATGGAGAAAACAATT
GACGGCCGCCATAGAGCCCACCGCATCCCCAGCATGCCTGCTATT
GTCTTCCCAATCCTCCCCCTTGCTGTCCTGCCCCACCCCACCCCC
CAGAATAGAATGACACCTACTCAGACAATGCGATGCAATTTCCTC
ATTTTATTAGGAAAGGACAGTGGGAGTGGCACCTTCCAGGGTCAA
GGAAGGCACGGGGGAGGGGCAAACAACAGATGGCTGGCAACTAGA
AGGCACAGGGTACCAACGCGTCGTAACTATAACGGTCCTAAGGTA
GCGAAAAGCACTCTCACAGCACCAGCACTAATCAGAGTGTGAAGA
GGGCCAAGTGCCGAACGAGTATATATAGGAATAAAAAATGACGTA
AATGTGTAAAGGTCAGAAAACGCCCAGAAAAATACACAGACCAAC
GCCCGAAACGAAAACCCGCGAAAAAATACCCAGAACTTCCTCAAC
AACCGCCACTTCCGCTTTCTCACGGTACGTCACTTCCGCAAGAAA
AGCAAAACTACATTTCCCACATGTGTAAAAACGAAACCCCGCCCC
TTGTAACCGCCCACAACTTACATCATCAAAACGTAAACTCCTACG
TCACCCGCCCCGCCTCTCCCCGCCCACCTCATTATCATATTGGCC
ACAATCCAAAATAAGGTATATTATTGATGATG

SEQUENCE ID NO. 119:
GC46 Gorilla Adenovirus Vector-Full Length Genome
CATCATCAATAATATACCTTATTTTGGATTGTGGCCAATATGATAA
TGAGGTGGGCGGGGAGAGGCGGGGGGGGTGACGTAGGACGCGCGA
GTAGGGTTGGGAGGTGTGGCGGAAGTGTGGCATTTGCAAGTGGGA
GGAGCTCACATGCAAGCTTCCGTCGCGGAAAATGTGACGTTTTTG
ATGAGCGCCGCCTACCTCCGGAAGTGCCAATTTTCGCGCGCTTTT
CACCGGATATCGTAGTAATTTTGGGCGGGACCATGTAAGATTTGG
CCATTTTCGCGCGAAAAGTGAAACGGGGAAGTGAAAACTGAATAA
TAGGGCGTTAGTCATAGTGCGTAATATTTACCGAGGGCCGAGGGA
CTTTGACCGATTACGTGGAGGACTCGCCCAGGTGTTTTTTACGTG
AATTTCCGCGTTCCGGGTCAAAGTCTCCGTTTTATTGTCACCGTC
ATTTGACGCGGCCGCCTAGGAACACCCACTACACCCCCACTCAAG
GGAACACCGTGCCCAGCTACCCTGAGACAGCCCCACTCCCAGGAA
ATGCAGGTGCCAACCAGCCCCAGTGAGGCATGGCACCTGAGCCAG
ACACCCCCAAATAGTCCCTTCCCACCTCCTCAAGCATCTGCACTC
TGGGTGAACCCAGAAAAACTGGGGCACCTGGGGAGACATAGAAGG
GCCAGGCCAGGAACTCCCCAATAAGCAGGAACAGAGACCTGACCC
CTGAGCCTCCCCCACCCTCTAAGGCTGCTCAATGTCAAGGCAGGA
GGGATCTCCACACACCTGCACTCTGGGTAATTCCCAACTTGCCCC
TTGCTCCATACCACCCCCCTCCACCCCATAATATTATAGAAGGAC
ACCTAGTCAGACAAAATGATGCAACTTAATTTTATTAGGACAAGG
CTGGTGGGCACTGGAGTGGCAACTTCCAGGGCCAGGAGAGGCACT
GGGGAGGGGTCACAGGGATGCCACCCATCAATCGATTCAGTTTCG
TATCTTCATTGTCATGTAGGCTTCTATGTAGTTGATGAAGATGTC
AAACTCACTCATGGCTTTGTAGATGCCTTTCTCTTGGAGCTTGTT
GAAGGCATTCTTCACTTGCTCCACGGCCTTGCTCTTGTTTTCACA
GGGAAGGAATCTGTGACAGCGCCGTAGTCTCAGTCTGAGGGTTTT
CAGGTTCTCCCCCAGGGAGTTCACATGCGCCTTGATGTCTGGGTC
TTGGTTCTCAGCTTGGGGCATCACCTCCTCCAGGTAAAACTGGAT
CATCTCAGACAAGGCTTGGCAACCCAGGTAACCCTTAAAGTCCTC
CAGCAAGGACTCCTTTAACAACAAGTTGTCCAGCTGATCCTTCAT
TTGAAAGAAGGTTTTCACTCTGCTGAAGGCATCTCTCAGGTCTCT
AAGCATGTTGGGGAGGTTGCCTGGGAAGTGGGTGCAGCTGTTCTC
AGACTGGGTGCCCTGGCCTGGGCTGGCCCTCACCCCAGTCAGGAG
GACCAGGCAACAGAGCAGGGCGCTGCTGTGCATGGTGGCGCTAGC
GGCTCTCAATTCCTTTTTTCCAAAGTAGAGGACAAGAAGTGTCCG
GCAACGAAGGTACCCAGCAAGGTTTTTCCTGTCAGGAACAAACAT
TTGTTGTAAATGTCATCCTAACATCAGCACACAAAAGGGATTAGC
AAAAAAAAAAAAAAAGAGCATGTTAAGATTACTCACCTTCTTCGC
CTGCAGGTTCTATGGAGGTCAAAACACTCGGCTCGGGATCCGTCT
CCAGAATGAGGGTTCACTAAACGTCGACGATCTCTATCACTGATA
GGGAGATCTCTATCACTGATAGGGAGAGCTCTGCTTATATAGACC
TCGCTCGTGATACGTCACTTCCTGGTGGATCACGCGCGATCCAAC
AAGTGCGGCGGACAGGCGAGCGCCCGGGCTTGAGAACGGTACTTT
CCCATTAATCAGCTATGGGAAAGTACCGTTTAAAGGTCACGTTGC
ATTAGTTTCAATAGCCCATTGACGTCAATGGTGGGAAAGTACATG
GCGTTTCAATTAAATTGGCTGGAAAAACCCAATGACTCACCCCTA
TTGACCTTATGTACGTGCCAATAATGGGAAAAACCCATTGACTCA
CCCCCTATTGACCTTTTGTACTGGGCAAAACCCAATGGAAAGTCC
CTATTGACTCAGTGTACTTGGCTCCAATGGGACTTTCCTGTTGCC
TATTCACCCTATTGACCTTATGTACTGGGCAAAACCCATGGAAAG
TCCCTAATGACTCAGTATACGTGCCAGTAATGGGAAAAACCCATT
GGCTTACCTCCCATTGACCTTATTGGTACTGGGCAAAACCCATGG
AAAGTCCCCTATTGACTCAATGTACTTGGCTCCAATGGAACTTTC
CTGTTGACTCACCCCCTATTGACCTTATGTACTGGGCAAAACCCA
ATGGAAAGTCCCTATTGAGTCAGTGTACTTGGCTCCAATGGGTTT
TTCCCATTGACTCATCCCCTATTGACCTTATGTACTGGGCAAAAC
CCAATGGAAAGTCCCTATTGACGCAGTGTACTTGGCTCCAATGGG
ACTTTTCCGTTGATTCACCTATTGACCTTATGTACGGGCAGGTAC
AATGGTACCCCTGCATTATGGATAGAGAAATGTTCTGGCACCTGG
ACTTGCACTGGGGACAGCCTATTTTGCTAGTTTGTTTTGTTTCGT
TTTGTTTTGATGGAGAGCGTATGTTAGTACTATGGATTCACACAA
AAAACCAACACACAGATGTAATGAAAATAAAGATATTTTATTGCG
GCACGCGTAGGCCTGACCATCTGGTGCTGGCCTGCACCAGGGCCG
AGTTTGGGTCTAGCGATGAGGATACCGATTGAGGTGGGTAAGGTG
GGCGTGGCTAGAAGGGTGGGGCGTGTATAAATTGGGGGTCTAAGG
GTCTCTCTGTTTTGTCTTGCAACAGCCGCCGCCATGAGCGACACC
GGCAACAGCTTTGATGGAAGCATCTTTAGCCCCTATCTGACAGTG
CGCATGCCTCACTGGGCTGGAGTGCGTCAGAATGTGATGGGTTCC
AACGTGGATGGACGCCCCGTTCTGCCTTCAAATTCGTCTACAATG
GCCTACGCGACCGTGGGAGGAACTCCGCTGGACGCCGCGACCTCC
GCCGCCGCCTCCGCCGCCGCCGCGACCGCGCGCAGCATGGCTACG
GACCTTTACAGCTCTTTGGTGGCGAGCGGCGCGGCCTCTCGCGCG
TCTGCTCGGGATGAGAAACTGACCGCTCTGCTGCTTAAACTGGAA
GACTTGACCCGGGAGCTGGCTCAACTGACCCAGCAGGTCTCCAGC
TTGCGTGAGAGCAGCCTTGCCTCCCCCTAATGGCCCATAATATAA
ATAAAAGCCAGTCTGTTTGGATTAAGCAAGTGTATGTTCTTTATT
TAACTCTCCGCGCGCGGTAAGCCCGGGACCAGCGGTCTCGGTCGT
TTAGGGTGCGGTGGATTCTTTCCAACACGTGGTACAGGTGGCTCT
GGATGTTTAGATACATGGGCATGAGTCCATCCCTGGGGTGGAGGT
AGCACCACTGCAGAGCTTCGTGCTCGGGGGTGGTGTTGTATATGA
TCCAGTCGTAGCAGGAGCGCTGGGCGTGGTGCTGAAAAATGTCCT
TAAGCAAGAGGCTTATAGCTAGGGGGAGGCCCTTGGTGTAAGTGT
TTACAAATCTGCTCAGTTGGGAGGGGTGCATCCGGGGGGATATAA
TGTGCATCTTGGACTGGATTTTTAGGTTGGCTATGTTCCCACCCA
GATCCCTTCTGGGATTCATGTTGTGCAGGACCACCAGCACGGTAT
ATCCAGTACACTTGGGAAATTTATCGTGGAGCTTAGACGGGAATG
CATGGAAGAACTTGGAGACGCCCTTGTGGCCTCCCAGATTTTCCA
TACATTCGTCCATGATGATGGCAATGGGCCCGTGGGAAGCTGCCT
GAGCAAAAATGTTTCTGGGATCGCTCACATCGTAGTTATGTTCCA
GGGTGAGGTCATCATAGGACATCTTTACAAATCGGGGGCGGAGGG
TCCCGGACTGGGGGATGATGGTGCCCTCGGGCCCCGGGGCGTAGT
TCCCCTCACAGATCTGCATCTCCCAGGCTTTCATTTCAGAGGGAG
GGATCATATCCACCTGCGGAGCGATGAAAAACACAGTTTCTGGCG
CAGGGGAGATTAACTGGGATGAGAGCAGGTTTCTGAGCAGCTGTG
ACTTTCCACAGCCGGTGGGCCCATATATCACGCCTATCACCGGCT
GCAGCTGGTAGTTAAGAGAGCTGCAGCTGCCGTCCTCCCGGAGCA
GGGGGGCCACCTCGTTCAGCATATCCCTGACGTGGATGTTCTCCC
TGACCAATTCCGCCAGAAGGCGCTCGCCGCCCAGCGAAAGCAGCT
CTTGCAAGGAAGCAAAATTTTTCAGCGGTTTTAGGCCGTCGGCCG
TGGGCATGTTTTTCAGCGTCTGGGTCAGCAGTTCCAGTCTGTCCC
ACAGCTCGGTGATGTGCTCTACGGCATCTCGATCCAGCAGATCTC
CTCGTTTCGCGGGTTGGGGCGGCTTTCGCTGTAGGGCACCAGCCG
ATGGGCGTCCAGCGGGGCCAGAGTCATGTCCTTCCATGGGCGCAG
GGTCCTCGTCAGGGTGGTCTGGGTCACGGTGAAGGGGTGCGCTCC
GGGTTGGGCGCTGGCCAGGGTGCGCTTGAGGCTGGTTCTGCTGGT
GCTGAATCGCTGCCGCTCTTCGCCCTGCGCGTCGGCCAGGTAGCA
TTTGACCATGGTCTCGTAGTCGAGACCCTCGGCGGCGTGCCCCTT
GGCGCGGAGCTTTCCCTTGGAGGTGGCGCCGCACGAGGGGCACTG
CAGGCTCTTCAGGGCGTAGAGCTTGGGAGCGAGAAACACGGACTC
TGGGGAGTAGGCGTCCGCGCCGCAGGAAGCGCAGACCGTCTCGCA
TTCCACCAGCCAAGTGAGCTCCGGGCGGTCAGGGTCAAAAACCAG
GTTGCCCCCATGCTTTTTGATGCGTTTCTTACCTCGGCTCTCCAT
GAGGCGGTGTCCCTTCTCGGTGACGAAGAGGCTGTCCGTGTCTCC
GTAGACCGACTTCAGGGGCCTGTCTTCCAGCGGAGTGCCTCTGTC
CTCCTCGTAGAGAAACTCTGACCACTCTGAGACGAAGGCCCGCGT
CCAGGCCAGGACGAAGGAGGCCACGTGGGAGGGGTAGCGGTCGTT
GTCCACTAGCGGGTCCACCTTCTCCAGGGTGTGCAGGCACATGTC
CCCCTCCTCCGCGTCCAGAAAAGTGATTGGCTTGTAGGTGTAGGA
CACGTGACCGGGGGTTCCCGACGGGGGGGTATAAAAGGGGGTGGG
CACCCTTTCATCTTCACTCTCTTCCGCATCGCTGTCTGCGAGAGC
CAGCTGCTGGGGTAAGTATTCCCTCTCGAAGGCGGGCATGACCTC
AGCGCTCAGGTTGTCAGTTTCTAAAAATGAGGAGGATTTGATGTT
CACCTGTCCGGAGGTGATACCTTTGAGGGTACCTGGGTCCATCTG
GTCAGAAAACACTATTTTTTTGTTGTCAAGCTTGGTGGCGAACGA
CCCGTAGAGGGCGTTGGAGAGCAGCTTGGCGATGGAGCGCAGGGT
CTGGTTTTTGTCGCGGTCGGCTCGCTCCTTGGCCGCGATGTTGAG
TTGCACGTACTCGCGGGCCACGCACTTCCACTCGGGGAAGACGGT
GGTGCGCTCGTCTGGGATCAGGCGCACCCTCCAGCCTCGGTTGTG
CAGGGTGACCATGTCGACGCTGGTGGCGACCTCGCCGCGCAGGCG
CTCGTTGGTCCAGCAGAGGCGGCCGCCCTTGCGCGAGCAGAAGGG
GGGTAGGGGGTCCAGCTGGTCCTCGTTTGGGGGGTCCGCGTCGAT
GGTGAAGACCCCGGGGAGCAAGCGCGGGTCAAAGTAGTCGATCTT
GCAAGCTTGCATGTCCAGAGCCCGCTGCCATTCGCGGGCGGCGAG
CGCGCGCTCGTAGGGGTTGAGGGGGGGGCCCCAGGGCATGGGGTG
GGTGAGCGCGGAGGCGTACATGCCGCAGATGTCATACACGTACAG
GGGTTCCCTGAGGATGCCGAGGTAGGTGGGGTAGCAGCGCCCCCC
GCGGATGCTGGCGCGCACGTAGTCATAGAGCTCGTGGGAGGGGGC
CAGCATGTTGGGCCCGAGGTTGGTGCGCTGGGGGCGCTCGGCGCG
GAAGGCGATCTGCCTGAAGATGGCATGGGAGTTGGAGGAGATGGT
GGGCCGCTGGAAGACGTTGAAGCTTGCTTCTTGCAAGCCCACCGA
GTCCCTGACGAAGCAGGCGTAGGACTCGCGCAGCTTGTGCACCAG
CTCGGCGGTGACCTGGACGTCGAGCGCGCAGTAGTCGAGGGTCTC
GCGGATGATGTCATACTTATCCTCCCCCTTCTTTTTCCACAGCTC
GCGGTTGAGGACGAACTCTTCGCGGTCTTTCCAGTACTCTTGGAG
GGGAAACCCGTCCGTGTCCGAACGGTAAGAGCCTAGCATGTAGAA
CTGGTTGACGGCCTGGTAGGGGCAACAGCCCTTCTCCACGGGCAG
CGCGTAGGCCTGCGCCGCCTTGCGGAGGGAGGTGTGGGTGAGGGC
GAAAGTGTCCCTGACCATGACTTTGAGGTATTGATGTTTGAAGTC
TGTGTCATCGCAGCCGCCCTGTTCCCACAGGGTGTAGTCCGTGCG
CTTTTTGGAGCGCGGGTTGGGCAGGGAGAAGGTGAGGTCATTGAA
GAGGATCTTCCCCGCTCGAGGCATGAAGTTTCTGGTGATGCGAAA
GGGCCCTGGGACCGAGGAGCGGTTGTTGATGACCTGGGCGGCCAG
GACGATCTCGTCAAAGCCGTTTATGTTGTGGCCCACGATGTAGAG
CTCCAAAAAGCGGGGCTGGCCCTTGATGGAGGGGAGCTTTTTGAG
TTCCTCGTAGGTGAGCTCCTCGGGCGATTCCAGGCCGTGCTCCTC
CAGGGCCCAGTCTTGCAAGTGAGGGTTGGCCGCCAGGAAGGATCG
CCAGAGGTCGCGGGCCATGAGGGTCTGCAGGCGGTCGCGGAAGGT
TCTGAACTGTCGCCCCACGGCCATCTTTTCGGGGGTGATGCAGTA
GAAGGTGAGGGGGTCTTTCTCCCAGGGGTCCCATCTGAGCTCTCG
GGCGAGGTCGCGCGCGGCGGCGACCAGAGCCTCGTTGCCCCCCAG
TTTCATGACCAGCATGAAGGGCACGAGCTGCTTGCCAAAGGCTCC
CATCCAAGTGTAGGTCTCTACATCGTAGGTGACAAAGAGGCGCTC
CGTGCGAGGATGAGAGCCGATCGGGAAGAACTGGATCTCCCGCCA
CCAGTTGGAGGATTGGCTGTTGATGTGGTGAAAGTAGAAGTCCCG
TCTGCGGGCCGAGCACTCGTGCTGGCTTTTGTAAAAGCGACCGCA
GTACTGGCAGCGCTGCACGGGTTGTATATCTTGCACGAGGTGAAC
CTGGCGACCTCTGACGAGGAAGCGCAGCGGGAATCTAAGTCCCCC
GCCTGGGGTCCCGTGTGGCTGGTGGTCTTCTACTTTGGTTGTCTG
GCCGCCAGCATCTGTCTCCTGGAGGGCGATGGTGGAGCAGACCAC
CACGCCGCGAGAGCCGCAGGTCCAGATCTCGGCGCTCGGCGGGCG
GAGTTTGATGACGACATCGCGCACATTGGAGCTGTCCATGGTCTC
CAGCTCCCGCGGCGGCAGGTCAGCTGGGAGTTCCTGGAGGTTCAC
CTCGCAGAGACGGGTCAAGGCGCGGGCAGTGTTGAGATGGTATCT
GATTTCAAGGGGCGTGTTGGCGGCGGAGTCGATGGCTTGCAGGAG
GCCGCAGCCCCGGGGGGCCACGATGGTTCCCCGCGGGGCGCGAGG
GGAGGCGGAAGCTGGGGGTGTGTTCAGAAGCGGTGACGCGGGCGG
GCCCCCGGAGGTAGGGGGGGTTCCGGCCCCACAGGCATGGGCGGC
AGGGGCACGTCTTCGCCGCGCGCGGGCAGGGGCTGGTGCTGGCTC
CGAAGAGCGCTTGCGTGCGCGACGACGCGACGGTTGGTGTCCTGT
ATCTGACGCCTCTGAGTGAAGACCACGGGTCCCGTGACCTTGAAC
CTGAAAGAGAGTTCGACAGAATCAATCTCGGCATCGTTGACAGCG
GCCTGGCGCAGGATCTCCTGCACGTCGCCCGAGTTGTCCTGGTAG
GCGATCTCTGCCATGAACTGCTCGATCTCTTCTTCCTGGAGATCT
CCTCGTCCGGCGCGCTCCACGGTGGCCGCCAGGTCGTTGGAGATG
CGACCCATGAGCTGCGAGAAGGCGTTGAGCCCGCCCTCGTTCCAG
ACCCGGCTGTAGACCACGCCCCCCTCGGCGTTGCGGGCGCGCATG
ACCACCTGGGCCAGGTTGAGCTCCACGTGTCGCGTGAAGACGGCG
TAGTTGCGCAGGCGCTGGAAAAGGTAGTTCAGGGTGGTGGCGGTG
TGCTCGGCGACGAAGAAGTACATGACCCAGCGCCGCAACGTGGAT
TCATTGATGTCCCCCAAGGCCTCCAGGCGCTCCATGGCCTCGTAG
AAGTCCACGGCGAAGTTGAAAAACTGGGAGTTGCGAGCGGACACG
GTCAACTCCTCCTCCAGAAGACGGATGAGCTCGGCGACAGTGTCG
CGCACCTCGCGCTCGAAGGCCACGGGGGGCGCTTCTTCCTCTTCC
ACCTCTTCTTCCATGATCGCTTCTTCTTCTTCCTCAGCCGGGACG
GGAGGGGGCGGCGGCGGCGGGGGAGGGGCGCGGCGGCGGCGGCGG
CGCACCGGGAGGCGGTCGATGAAGCGCTCGATCATCTCCCCCCGC
ATGCGGCGCATGGTCTCGGTGACGGCGCGGCCGTTCTCCCGGGGG
CGCAGCTCGAAGACGCCGCCTCTCATCTCGCCGCGGGGCGGGCGG
CCGTGAGGTAGCGAGACGGCGCTGACTATGCATCTTAACAATTGC
TGTGTAGGTACACCGCCGAGGGACCTGATTGAGTCCAGATCCACC
GGATCCGAAAACCTTTGGAGGAAAGCGTCTATCCAGTCGCAGTCG
CAAGGTAGGCTGAGCACCGTGGCGGGCGGGGGCGGGTCTGGAGAG
TTCCTGGCGGAGATGCTGCTGATGATGTAATTAAAGTAGGCGGTC
TTGAGAAGGCGGATGGTGGACAGGAGCACCATGTCTTTGGGTCCG
GCCTGTTGGATGCGGAGGCGGTCGGCCATGCCCCAGGCCTCGTTC
TGACACCGGCGCAGGTCTTTGTAGTAGTCTTGCATGAGTCTTTCC
ACCGGCACCTCTTCTCCTTCCTCTTCTCCATCTCGCCGGTGGTTT
CTCGCGCCGCCCATGCGCGTGACCCCAAAGCCCCTGAGCGGCTGC
AGCAGGGCCAGGTCGGCGACCACGCGCTCGGCCAAGATGGCCTGC
TGCACCTGAGTGAGGGTCCTCTCGAAGTCATCCATGTCCACGAAG
CGGTGGTAGGCGCCCGTGTTGATGGTGTAGGTGCAGTTGGCCATG
ACGGACCAGTTGACGGTCTGGTGTCCCGGCTGCGAGAGCTCCGTG
TACCGCAGGCGCGAGAAGGCGCGGGAATCGAACACGTAGTCGTTG
CAAGTCCGCACCAGATACTGGTAGCCCACCAGGAAGTGCGGCGGA
GGTTGGCGATAGAGGGGCCAGCGCTGGGTGGCGGGGGCGCCGGGC
GCCAGGTTTTCCAGCATGAGGCGGTGGTATCCGTAGATGTACCTG
GACATCCAGGTGATGCCGGCGGCGGTGGTGGTGGCGCGCGCGTAG
TCGCGGACCCGGTTCCAGATGTTTCGCAGGGGCGAGAAGTGTTCC
ATGGTCGGCACGCTCTGGCCGGTGAGGCGCGCGCAGTCGTTGACG
CTCTATACACACACAAAAACGAAAGCGTTTACAGGGCTTTCGTTC
TGTAGCCTGGAGGAAAGTAAATGGGTTGGGTTGCGGTGTGCCCCG
GTTCGAGACCAAGCTGAGCTCGGCCGGCTGAAGCCGCAGCTAACG
TGGTATTGGCAGTCCCGTCTCGACCCAGGCCCTGTATCCTCCAGG
ATACGGTCGAGAGCCCTTTTGCTTTCTTGGCCAAGCGCCCGTGGC
GCGATCTGGGATAGATGGTCGCGATGAGAGGACAAAAGCGGCTCG
CTTCCGTAGTCTGGAGAAACAATCGCCAGGGTTGCGTTGCGGCGT
ACCCCGGTTCGAGCCCCTATGGCGGCTTGAATCGGCCGGAACCGC
GGCTAACGAGGGCCGTGGCAGCCCCGTCCTCAGGACCCCGCCAGC
CGACTTCTCCAGTTACGGGAGCGAGCCCCTTTTGTTTTTTATTTT
TTAGATGCATCCCGTGCTGCGGCAGATGCGCCCCTCGCCCCGGCC
CGATCAGCAGCAGCAACAGCAGGCATGCAGACCCCCCTCTCCCCT
TTCCGCCCCGGTCACCACGGCCGCGGCGGCCGTGTCGGGCGCGGG
GGGCGCGCTGGAGTCAGATGAGCCACCGCGGCGGCGACCTAGGCA
GTATCTGGACTTGGAAGAGGGCGAGGGACTGGCGCGGCTGGGGGC
GAACTCTCCAGAGCGCCACCCGCGGGTGCAGTTGAAAAGGGACGC
GCGCGAGGCGTACCTGCCGCGGCAGAACCTGTTTCGCGACCGCGG
GGGCGAGGAGCCCGAGGAGATGCGAGACTGCAGGTTCCAAGCGGG
GCGCGAGCTGCGGCGCGGGCTGGACAGACAGCGCCTGCTGCGCGA
GGAGGACTTTGAGCCCGACACGCAGACGGGCATCAGCCCCGCGCG
CGCGCACGTAGCCGCGGCCGACCTGGTGACCGCCTACGAGCAGAC
GGTGAACCAGGAGCGCAACTTCCAAAAGAGCTTCAACAACCACGT
GCGCACGCTGGTGGCGCGCGAGGAGGTGACCCTGGGTCTCATGCA
TCTGTGGGACCTGGTGGAGGCGATCGTGCAGAACCCCAGCAGCAA
GCCCCTGACCGCGCAGCTGTTCCTGGTGGTGCAGCACAGCAGGGA
CAACGAGGCCTTCAGGGAGGCGCTGCTGAACATCACCGAGCCGGA
GGGGCGCTGGCTCCTGGACCTGATAAACATCCTGCAGAGCATAGT
GGTGCAGGAGCGCAGCCTGAGCCTGGCCGAGAAGGTGGCGGCCAT
CAACTACTCTATGCTGAGCCTGGGCAAGTTCTACGCCCGCAAGAT
CTACAAGACCCCCTACGTGCCCATAGACAAGGAGGTGAAGATAGA
CAGCTTCTACATGCGCATGGCGCTGAAGGTGCTGACCCTGAGCGA
CGACCTGGGAGTGTACCGCAACGAGCGCATCCACAAGGCCGTGAG
CGCCAGCCGGCGGCGCGAGCTGAGCGACCGCGAGCTGATGCACAG
TCTGCAGCGCGCGCTGACCGGCGCGGGCGAGGGCGACAGGGAGGT
CGAGTCCTACTTCGACATGGGGGCCGACCTGCACTGGCAGCCGAG
CCGCCGCGCCCTGGAGGCGGCGGGGGCGTACGGCGGCCCCCTGGC
GGCCGATGACCAGGAAGAGGAGGACTATGAGCTAGAGGAGGGCGA
GTACCTGGAGGACTGACCTGGCTGGTGGTGTTTTGGTATAGATGC
AAGATCCGAACGTGGCGGACCCGGCGGTCCGGGCGGCGCTGCAAA
GCCAGCCGTCCGGCATTAACTCCTCTGACGACTGGGCCGCGGCCA
TGGGTCGCATCATGGCCCTGACCGCGCGCAACCCCGAGGCTTTCA
GGCAGCAGCCTCAGGCCAACCGGCTGGCGGCCATCTTGGAAGCGG
TAGTGCCCGCGCGCTCCAACCCCACCCACGAGAAGGTGCTGGCCA
TAGTCAACGCGCTGGCGGAGAGCAGGGCCATCCGCGCGGACGAGG
CCGGACTGGTGTACGATGCGCTGCTGCAGCGGGTGGCGCGGTACA
ACAGCGGCAACGTGCAGACCAACCTGGACCGCCTGGTGACGGACG
TGCGCGAGGCCGTGGCGCAGCGCGAGCGCTTGCATCAGGACGGTA
ACCTGGGCTCGCTGGTGGCGCTAAACGCCTTCCTCAGCACCCAGC
CGGCCAACGTACCGCGGGGGCAGGAGGACTACACCAACTTTTTGA
GCGCGCTGCGGCTGATGGTGACCGAGGTCCCTCAGAGCGAGGTGT
ACCAGTCGGGGCCCGACTACTTCTTCCAGACCAGCAGACAGGGCT
TGCAAACCGTGAACCTGAGCCAGGCTTTCAAGAACCTGCGGGGGC
TGTGGGGAGTGAAGGCGCCCACCGGCGACCGGGCTACGGTGTCCA
GCCTGCTAACCCCCAACTCGCGCCTGCTGCTGCTGCTGATCGCGC
CCTTCACGGACAGCGGGAGCGTCTCGCGGGAGACCTATCTGGGCC
ACCTGCTGACGCTGTACCGCGAGGCCATCGGGCAGGCGCAGGTGG
ACGAGCACACCTTCCAAGAGATCACCAGCGTGAGCCACGCGCTGG
GGCAGGAGGACACGGGCAGCCTGCAGGCGACCCTGAACTACCTGC
TGACCAACAGGCGGCAGAAGATTCCCACGCTGCACAGCCTGACCC
AGGAGGAGGAGCGCATCTTGCGCTACGTGCAGCAGAGCGTGAGCC
TGAACCTGATGCGCGACGGCGTGACGCCCAGCGTGGCGCTGGACA
TGACCGCGCGCAACATGGAACCGGGCATGTACGCCTCCCACCGGC
CGTTCATCAACCGCCTGATGGACTACTTGCATCGGGCGGCGGCCG
TGAACCCCGAGTACTTCACTAATGCCATTCTGAATCCCCACTGGA
TGCCCCCTCCGGGTTTCTACAACGGGGACTTTGAGGTGCCCGAGG
TCAACGACGGGTTCCTCTGGGATGACATGGATGACAGTGTGTTCT
CACCCAACCCGCTGCGCGCCGCGTCTCTGCGATTGAAGGAGGGCT
CTGACAGGGAAGGACCGAGAAGTCTGGCCTCCTCCCTGGCTCTGG
GAGCGGTGGGCGCCACGGGCGCGGCGGCGCGGGGCAGTAGCCCCT
TCCCCAGCCTGGCAGACTCTCTGAACAGCGGGCGGGTGAGCAGGC
CCCGCTTGCTAGGCGAGGAGGAGTATCTGAACAACTCCCTGCTGC
AGCCCGCGAGGGACAAGAACGCTCAGCGGCAGCAGTTTCCCAACA
ATGGGATAGAGAGCCTGGTGGACAAGATGTCCAGATGGAAGACGT
ATGCGCAGGAGTACAAGGAGTGGGAGGACCGCCAGCCGCGGCCCT
TGCCGCCCCCTAGGCAGCGCTGGCAGCGGCGCGCGTCCAACCGCC
GCTGGAGGCAGGGGCCCGAGGACGATGATGACTCTGCAGATGACA
GCAGCGTGTTGGACCTGGGCGGGAGCGGGAACCCCTTTTCGCACC
TGCGCCCACGCCTGGGCAAGATGTTTTAAAAGAAAAAAAAAAAAA
TAAAACTCACCAAGGCCATGGCGACGAGCGTTGGTTTTTTGTTCC
CTTCCTTAGTATGCGGCGCGCGGCGATGTTCGAGGAGGGGCCTCC
CCCCTCTTACGAGAGCGCGATGGGGATTTCTCCTGCGGCGCCCCT
GCAGCCTCCCTACGTGCCTCCTCGGTACCTGCAACCTACAGGGGG
GAGAAATAGCATCTGTTACTCTGAGCTGCAGCCCCTGTACGATAC
CACCAGACTGTACCTGGTGGACAACAAGTCCGCGGACGTGGCCTC
CCTGAACTACCAGAACGACCACAGCGATTTTTTGACCACGGTGAT
CCAAAACAACGACTTCACCCCAACCGAGGCCAGCACTCAGACCAT
AAACCTGGATAACAGGTCGAACTGGGGCGGCGACCTGAAGACCAT
CTTGCACACCAACATGCCCAACGTGAACGAGTTCATGTTCACCAA
CTCTTTTAAGGCGCGGGTGATGGTGGCGCGCGAGCAGGGGGAGGC
GAAGTACGAGTGGGTGGACTTCACGCTGCCCGAGGGCAACTACTC
AGAGACCATGACTCTCGACCTGATGAACAATGCGATCGTGGAACA
CTATCTGAAAGTGGGCAGGCAGAACGGGGTGAAGGAAAGCGATAT
CGGGGTCAAGTTTGACACCAGAAACTTCCGTCTGGGCTGGGACCC
CGTGACCGGGCTGGTCATGCCGGGGGTCTACACCAACGAGGCCTT
TCATCCCGACATAGTGCTTCTGCCCGGCTGTGGGGTGGACTTCAC
CCAGAGCCGGCTGAGCAACCTGCTGGGCATTCGCAAGCGGCAGCC
TTTCCAGGAGGGTTTCAAGATCACCTATGAGGATCTGAAGGGGGG
CAACATTCCCGCGCTCCTTGATCTGGACGCCTACGAGGAGAGCTT
GAAACCCGAGGAGAGCGCTGGCGACAGCGGCGAGAGTGGCGAGGA
GCAAGCCGGCGGCGGTGGCGGCGCGTCGGTAGAAAACGAAAGTAC
GCCCGCAGTGGCGGCGGACGCTGCGGAGGTCGAGCCGGAGGCCAT
GCAGCAGGACGCAGAGGAGGGCGCACAGGAGGGCGCGCAGAAGGA
CATGAACGATGGGGAGATCAGGGGAGACACATTCGCCACCCGGGG
CGAAGAAAAAGAGGCAGAGGCGGCGGCGGCGGCGACGGCGGAGGC
CGAAACCGAGGTTGAGGCAGAGGCAGAGCCCGAGACCGAAGTTAT
GGAAGACATGAATGATGGAGAACGTAGGGGCGACACGTTCGCCAC
CCGGGGCGAAGAGAAGGCGGCGGAGGCAGAAGCCGCGGCTGAGGA
GGCGGCTGCGGCTGCGGCCAAGACTGAGGCTGCGGCTAAGGCTGA
GGTCGAAGCCAATGTTGCGGTTGAGGCTCAGGCTGAGGAGGAGGC
GGCGGCTGAAGCAGTTAAGGAAAAGGCCCAGGCAGAGCAGGAAGA
GAAAAAACCTGTCATTCAACCTCTAAAAGAAGATAGCAAAAAGCG
CAGTTACAACGTCATCGAGGGCAGCACCTTTACCCAGTACCGCAG
CTGGTACCTGGCGTACAACTACGGCGACCCGGTCAAGGGGGTGCG
CTCGTGGACCCTGCTCTGCACGCCGGACGTCACCTGCGGCTCCGA
GCAGATGTACTGGTCGCTGCCGAACATGATGCAAGACCCGGTGAC
CTTCCGCTCCACGCGGCAGGTTAGCAACTTCCCGGTGGTGGGCGC
CGAACTGCTGCCCGTGCACTCCAAGAGTTTTTACAACGAGCAGGC
CGTCTACTCCCAGCTGATCCGCCAGGCCACCTCTCTGACCCACGT
GTTCAATCGCTTTCCCGAGAACCAGATTTTGGCGCGCCCGCCGGC
CCCCACCATCACCACCGTGAGTGAAAACGTTCCTGCCCTCACAGA
TCACGGGACGCTACCGCTGCGCAACAGCATCTCAGGAGTCCAGCG
AGTGACCATTACTGACGCCAGACGCCGGACCTGCCCCTACGTTTA
CAAGGCCTTGGGCATAGTCTCGCCGCGCGTCCTCTCCAGTCGCAC
TTTTTAAAACACATCTACCCACACGTTCCAAAATCATGTCCGTAC
TCATCTCACCCAGCAACAACACCGGCTGGGGGCTGCGCGCGCCCA
GCAAGATGTTTGGAGGGGCGAGGAAGCGCTCCGACCAGCACCCTG
TGCGCGTGCGCGGCCACTACCGCGCGCCCTGGGGAGCGCACAAGC
GCGGGCGCACAGGGCGCACCACTGTGGACGACGTCATTGACTCCG
TAGTGGAGCAAGCGCGCCACTACACACCCGGCGCGCCGACCGCCC
CCGCCGTGTCCACCGTGGACCAGGCGATCGAAAGCGTGGTACAGG
GCGCGCGGCACTATGCCAACCTTAAAAGTCGCCGCCGCCGCGTGG
CCCGCCGCCATCGCCGGAGACCCCGGGCCACCGCCGCCGCGCGCC
TTACTAAGGCTCTGCTCAGGCGCGCCAGGCGAACTGGCCACCGGG
CCGCCATGAGGGCCGCACGGCGGGCTGCCGCTGCCGCAAGCGCCG
TGGCCCCGCGGGCACGAAGGCGCGCGGCCGCCGCCGCCGCCGCCG
CCATTTCCAGCTTGGCCTCGACGCGGCGCGGTAACATATACTGGG
TGCGCGACTCGGTAACCGGCACGCGGGTACCCGTGCGCTTTCGCC
CCCCGCGGAATTAGCACAAGACAACATACACACTGAGTCTCCTGC
TGTTGTGTATCCCAGCGGCGACCGTCAGCAGCGGCGACATGTCCA
AGCGCAAAATTAAAGAAGAGATGCTCCAGGTCATCGCGCCGGAGA
TCTATGGGCCCCCGAAGAAGGAGGAGGATGATTACAAGCCCCGCA
AGCTAAAGCGGGTCAAAAAGAAAAAGAAAGATGATGATGATGACG
AGGCGGTGGAGTTTGTCCGCCGCATGGCACCCAGGCGCCCCGTGC
AGTGGAAGGGCCGGCGCGTGCAGCGCGTTTTGCGCCCCGGCACCG
CGGTGGTCTTCACGCCCGGCGAGCGCTCCACGCGCACTTTCAAGC
GGGTGTACGATGAGGTGTACGGCGACGAGGACCTGTTGGAGCAGG
CCAACCAGCGCTTTGGGGAGTTTGCATATGGGAAACGGCCCCGCG
AGAGTCTAAAAGAGGACCTGCTGGCGCTACCGCTGGACGAGGGCA
ATCCCACCCCGAGTCTGAAGCCGGTAACCCTGCAACAGGTGCTGC
CTTTGAGCGCGCCCAGCGAGCATAAGCGAGGGTTGAAGCGCGAAG
GCGGGGACCTGGCGCCCACCGTGCAGTTGATGGTGCCCAAGCGGC
AGAAGCTGGAGGACGTGCTGGAGAAAATGAAAGTAGAGCCCGGGA
TCCAGCCCGAGATCAAGGTCCGCCCCATCAAGCAGGTGGCGCCCG
GCGTGGGAGTCCAGACCGTGGACGTTAGGATTCCCACGGAGGAGA
TGGAAACCCAAACCGCCACTCCCTCTTCGGCGGCCAGCGCCACCA
CCGGCACCGCTTCGGTAGAGGTGCAGACGGACCCCTGGCTACCCG
CCACCGCTGTTGCCGCCGCCGCCCCCCGTTCGCGCGGGCGCAAGA
GAAATTATCCAGCGGCCAGCGCGCTCATGCCCCAGTACGCACTGC
ATCCATCCATCGCGCCCACCCCCGGCTACCGCGGGTACTCGTACC
GCCCGCGCAGATCAGCCGGCACTCGCGGCCGCCGCCGCCGTGCGA
CCACAACCAGCCGCCGCCGTCGCCGCCGCCGCCAGCCAGTGCTGA
CCCCCGTGTCTGTAAGGAAGGTGGCTCGCTCGGGGAGCACGCTGG
TGGTGCCCAGAGCGCGCTACCACCCCAGCATCGTTTAAAGCCGGT
CTCTGTATGGTTCTTGCAGATATGGCCCTCACTTGTCGCCTCCGC
TTCCCGGTGCCGGGATACCGAGGAAGAACTCACCGCCGCAGAGGC
ATGGCGGGCAGCGGTCTCCGCGGCGGCCGTCGCCATCGCCGGCGC
GCAAAAAGCAGGCGCATGCGCGGCGGTGTGCTGCCTCTGCTAATC
CCGCTAATCGCCGCGGCGATCGGTGCCGTACCCGGGATCGCCTCC
GTGGCCCTGCAGGCGTCCCAGAAACGTTGACTCTTGCAACCTTGC
AAGCTTGCATTTTTTGGAGGAAAAAATAAAAAAAGTCTAGACTCT
CACGCTCGCTTGGTCCTGTGACTATTTTGTAGAAAAAAGATGGAA
GACATCAACTTTGCGTCGCTGGCCCCGCGTCACGGCTCGCGCCCG
TTCATGGGAGACTGGACAGATATCGGCACCAGCAATATGAGCGGT
GGCGCCTTCAGCTGGGGCAGTCTGTGGAGCGGCCTTAAAAATTTT
GGTTCCACCATTAAGAACTATGGCAACAAAGCGTGGAACAGCAGC
ACGGGCCAGATGCTGAGAGACAAGTTGAAAGAGCAGAACTTCCAG
GAGAAGGTGGCGCAGGGCCTGGCCTCTGGCATCAGCGGGGTGGTG
GACATAGCTAACCAGGCCGTGCAGAAAAAGATAAACAGTCATCTG
GACCCCCGTCCTCAGGTGGAGGAAATGCCTCCAGCGATGGAGACG
GTGTCTCCCGAGGGCAAAGGCGAAAAGCGCCCGCGGCCCGACAGA
GAAGAGACCCTGGTGTCACACACCGAGGAGCCGCCCTCTTACGAG
GAGGCAGTCAAGGCCGGCCTGCCCACCACTCGCCCCATAGCCCCC
ATGGCCACCGGTGTGGTGGGCCACAGGCAACACACTCCCGCAACA
CTAGATCTGCCCCCGCCGTCCGAGCCGCCGCGCCAGCCAAAGGCG
GCGACGGTGCCCGCTCCCTCCACTTCCGCCGCCAACAGAGTGCCC
CTGCGCCGCGCCGCGAGCGGCCCCCGGGCCTCGCGAGTTAGCGGC
AACTGGCAGAGCACACTGAACAGCATCGTGGGCCTGGGAGTGAGG
AGTGTGAAGCGCCGCCGTTGCTACTGAATGAGCAAGCTAGCTAAC
GTGTTGTATGTGTGTATGCGTCCTATGTCGCCGCCAGAGGAGCTG
TTGAGCCGCCGGCGCCGTCTGCACTCCAGCGAATTTCAAGATGGC
GACCCCATCGATGATGCCTCAGTGGTCGTACATGCACATCTCGGG
CCAGGACGCTTCGGAGTACCTGAGCCCCGGGCTGGTGCAGTTCGC
CCGCGCCACAGACACCTACTTCAACATGAGTAACAAGTTCAGGAA
CCCCACTGTGGCGCCCACCCACGATGTGACCACGGACCGGTCGCA
GCGCCTGACGCTGCGGTTCATCCCCGTGGATCGGGAGGACACCGC
CTACTCTTACAAGGCGCGGTTCACGCTGGCCGTGGGCGACAACCG
CGTGCTGGACATGGCCTCCACTTACTTTGACATCAGGGGGGTGCT
GGACAGGGGCCCCACCTTCAAGCCCTACTCGGGTACTGCCTACAA
CTCCCTGGCCCCCAAGGGCGCTCCCAATTCTTGCGAGTGGGAACA
AGAGGAAAATCAGGTGGTCGCTGCAGATGATGAACTTGAAGATGA
AGAAGCGCAAGCTCAAGAGGACGCCCCAGCTAAAAAAATTCATGT
ATATGCCCAGGCGCCTCTTGCTGGCGAAAAGATTACCAAGGATGG
TTTGCAAATAGGTACTGAAGTTGTAGGAGATACATCTAAGGACAC
TTTTGCAGACAAAACATTCCAACCCGAACCTCAGATAGGCGAGTC
TCAGTGGAACGAGGCTGATGCCACAGTAGCAGGAGGCAGAGTCTT
GAAAAAAACCACCCCTATGAGACCTTGCTATGGATCCTATGCCAG
GCCTACAAATGCCAACGGGGGTCAAGGAATTATGGTTGCCAATGA
ACAAGGAGTGTTGGAGTCTAAAGTGGAGATGCAATTTTTTTCTAA
CACTACAACCCTTAATGCGCGGGATGGAGCTGGCAATCCCGAACC
AAAGGTGGTGTTGTACAGTGAAGATGTCCACTTGGAATCTCCTGA
CACTCATTTGTCTTACAAGCCCAAAAAGGATGATGTTAATGCTAA
AATTATGTTGGGTCAGCAAGCTATGGCTAACAGGCCCAACCTCAT
TGCTTTTAGAGATAATTTCATTGGACTCATGTACTACAACAGCAC
TGGTAACATGGGAGTGCTGGCGGGTCAGGCCTCTCAGTTGAATGC
CGTGGTGGACCTGCAGGATAGAAACACAGAACTGTCATATCAGCT
TATGCTTGATTCCATTGGGGATAGATCCAGATACTTCTCCATGTG
GAACCAGGCAGTGGATAGCTATGACCCAGATGTCAGAATCATTGA
AAACCATGGTGTCGAGGACGAGCTACCCAACTACTGCTTCCCTCT
GGGCGGCATAGGAATTACTGATACTTATCAAGGGATCAAAAATAC
CAATGGCAATGGTCAGTGGACCAAAGATGATCAGTTCGCGGACCG
TAATGAAATAGGGGTGGGAAACAACTTCGCCATGGAGATCAACAT
CCAGGCCAACCTCTGGAGGAACTTCCTCTATGCGAACGTGGGGCT
CTACCTGCCAGACAAGCTCAAGTACAACCCCACCAACGTGGACAT
CTCTGACAACCCCAACACCTATGACTACATGAACAAGCGTGTGGT
GGCTCCCGGCCTGGTGGACTGCTTTGTCAATGTGGGAGCCAGGTG
GTCCCTGGACTACATGGACAACGTCAACCCCTTCAACCACCACCG
CAATGCGGGTCTGCGCTACCGCTCCATGATCCTGGGCAACGGGCG
CTACGTGCCCTTCCACATTCAGGTGCCCCAGAAGTTCTTTGCCAT
CAAGAACCTCCTCCTCCTGCCGGGCTCCTACACTTACGAGTGGAA
CTTCAGGAAGGATGTCAACATGGTCCTGCAGAGCTCTCTGGGCAA
TGACCTTAGGGTGGACGGGGCCAGCATCAAGTTTGACAGCGTCAC
CCTCTATGCTACCTTCTTCCCCATGGCTCACAACACCGCCTCCAC
GCTCGAGGCCATGCTGAGGAACGACACCAACGACCAGTCCTTCAA
TGACTACCTCTCTGGGGCCAACATGCTCTACCCCATCCCCGCCAA
GGCCACCAACGTGCCCATCTCCATTCCCTCTCGCAACTGGGCCGC
CTTCAGAGGCTGGGCCTTTACCCGCCTTAAGACCAAGGAAACCCC
CTCCCTGGGCTCGGGTTTTGACCCCTACTTTGTCTACTCGGGATC
CATCCCCTACCTGGATGGCACCTTCTACCTCAACCACACTTTTAA
GAAGATATCCATCATGTATGACTCCTCCGTCAGCTGGCCGGGCAA
TGACCGCCTGCTCACCCCCAATGAGTTCGAGGTCAAGCGCGCCGT
GGACGGCGAGGGCTACAACGTGGCCCAGTGCAACATGACCAAGGA
CTGGTTCCTGGTGCAGATGCTGGCCAACTACAACATAGGCTACCA
GGGCTTCTACATCCCAGAGAGCTACAAGGACAGGATGTACTCCTT
CTTCAGAAATTTCCAACCCATGAGCAGGCAGGTGGTGGACGAGAC
CAAATACAAGGACTATCAGGCCATTGGCATCACTCACCAGCACAA
CAACTCGGGATTCGTGGGCTACCTGGCTCCCACCATGCGCGAGGG
GCAGGCCTACCCCGCCAACTTCCCCTACCCGTTGATAGGCAAGAC
CGCGGTCGACAGCGTCACCCAGAAAAAGTTCCTCTGCGACCGCAC
CCTCTGGCGCATCCCCTTCTCTAGCAACTTCATGTCCATGGGTGC
GCTCACGGACCTGGGCCAGAACCTGCTCTATGCCAACTCCGCCCA
TGCGCTGGACATGACTTTTGAGGTGGACCCCATGGACGAGCCCAC
CCTTCTCTATATTGTGTTTGAAGTGTTCGACGTGGTCAGAGTGCA
CCAGCCGCACCGCGGTGTCATCGAGACCGTGTACCTGCGCACGCC
CTTCTCGGCCGGCAACGCCACCACCTAAGGAGACAGCGCCGCCGC
CTGCATGACGGGTTCCACCGAGCAAGAGCTCAGGGCCATCGCCAG
AGACCTGGGATGCGGACCCTATTTTTTGGGCACCTATGACAAACG
CTTCCCGGGCTTCATCTCCCGAGACAAGCTCGCCTGCGCCATCGT
CAACACGGCCGCGCGCGAGACCGGGGGCGTGCACTGGCTGGCCTT
TGGCTGGGACCCGCGCTCCAAAACCTGCTACCTCTTCGACCCCTT
TGGCTTCTCCGATCAGCGCCTCAGACAGATCTATGAGTTTGAGTA
CGAGGGGCTGCTGCGCCGCAGCGCGCTTGCCTCCTCGCCCGACCG
CTGCATCACCCTTGAGAAGTCCACCGAGACCGTGCAGGGGCCCCA
CTCGGCCGCCTGCGGTCTCTTCTGCTGCATGTTTTTGCACGCCTT
TGTGCGCTGGCCCCAGAGTCCCATGGATCGCAACCCCACCATGAA
CTTGCTCAAGGGAGTGCCCAACGCCATGCTCCAGAGCCCCCAGGT
CCAGCCCACCCTGCGCCACAACCAGGAACAGCTCTACCGCTTCCT
GGAGCGCCACTCCCCCTACTTCCGCAGTCACAGCGCGCACATCCG
GGGGGCCACCTCTTTCTGCCACTTGCAACAAAACATGCAAGACGG
AAAATGATGTACAGCTCGCTTTTTAATAAATGTAAAGACTGTGCA
CTTTATTTATACACGGGCTCTTTCTGGTTATTTATTCAACACCGC
CGTCGCCATCTAGAAATCGAAAGGGTTCTGCCGCGCGTCGCCGTG
CGCCACGGGCAGAGACACGTTGCGATACTGGAAGCGGCTCGCCCA
CTTGAACTCGGGCACCACCATGCGGGGCAGTGGCTCCTCGGGGAA
GTTCTCGCCCCACAGGGTGCGGGTCAGCTGCAGCGCGCTCAGGAG
GTCGGGAGCCGAGATCTTGAAGTCGCAGTTGGGGCCGGAACCCTG
CGCGCGCGAGTTGCGGTACACGGGGTTGCAGCACTGGAACACCAG
CAGGGCCGGATTACGCACGCTGGCCAGCAGGCTCTCGTCGCTGAT
CATGTCGCTGTCCAGATCCTCCGCGTTGCTCAGGGCGAATGGGGT
CATCTTGCAGACCTGCCTGCCCAGGAAAGGCGGCAGCCCGGGCTT
GCCGTTGCAGTCGCAGCGCAGGGGCATCAGCAGGTGCCCGTGGCC
CGTCTGCGCCTGCGGGTACAGCGCGCGCATGAAGGCTTCGATCTG
CCTGAAAGCCACCTGCGTCTTGGCTCCCTCCGAAAAGAACATCCC
ACAGGACTTGCTGGAGAACTGGTTCGCGGGACAGCTGGCATCGTG
CAGGCAGCAGCGCGCGTCGGTGTTGGCGATCTGCACCACGTTGCG
ACCCCACCGGTTCTTCACTATCTTGGCCTTGGAAGCCTGCTCCTT
CAGCGCGCGCTGGCCGTTCTCGCTGGTCACATCCATCTCTATCAC
CTGCTCCTTGTTGATCATGTTTGTCCCGTGCAGACACTTCAGGTC
GCCCTCCGTCTGGGTGCAGCGGTGCTCCCACAGCGCGCAACCGGT
GGGCTCCCAATTTTTGTGGGTCACCCCCGCGTAGGCCTGCAGGTA
GGCCTGCAAGAAGCGCCCCATCATGGCCACAAAGGTCTTCTGGCT
CGTAAAGGTCAGCTGCAGGCCGCGATGCTCTTCGTTCAGCCAGGT
CTTGCAGATGGCGGCCAGCGCCTCGGTCTGCTCGGGCAGCATCCT
AAAATTTGTCTTCAGGTCGTTATCCACGTGGTACTTGTCCATCAT
GGCGCGCGCCGCCTCCATGCCCTTCTCCCAGGCGGACACCATGGG
CAGGCTTAGGGGGTTTATCACTTCCACCGGCGAGGACACCGTACT
TTCGATTTCTTCTTCCTCCCCCTCTTCCCGGCGCGCGCCCACGCT
GCTGCGCGCTCTCACCGCCTGCACCAAGGGGTCGTCTTCAGGCAA
GCGCCGCACCGAGCGCTTGCCGCCCTTGACCTGCTTAATCAGCAC
CGGCGGGTTGCTGAAGCCCACCATGGTCAGCTCCGCCTGCTCTTC
TTCGTCTTCGCTGTCTACCACTATCTCTGGGGAAGGGCTTCTCCG
CTCTGCGGCGGTGCGCTTCTTTTTTTTCTTGGGAGCAGCCGTGAC
GGAGTCCGCCACGGCGACGGAGGTCGAGGGCGTGGGGCTGGGGGT
GCGCGGTACCAGGGCCTCGTCGCCCTCGGACTCTTCCTCTGACTC
CAGGCGGCGGCGGAGACGCTTCTTTGGGGGCGCGCGCGTCAGCGG
CGGCGGAGACGGGGACGGGGACGGGGACGGGACGCCCTCCACAGG
GGGTGGTCTTCGCGCAGACCCGCGGCCGCGCTCGGGGGTCTTTTC
GAGCTGGTCTTGGTCCCGACTGGCCATTGTATCCTCCTCCTCCTA
GGCAGAGAGACATAAGGAGTCTATCATGCAAGTCGAGAAGGAGGA
GAGCTTAACCACCCCCTCTGAGACCGCCGATGCGCCCGCCGTCGC
CGTCGCCCCCGCTGCCGCCGACGCGCCCGCCACACCGAGCGACAC
CCCCGCGGACCCCCCAGCCGACGCACCCCTGTTCGAGGAAGCGGC
CGTGGAGCAGGACCCGGGCTTTGTCTCGGCAGAGGAGGATTTGCG
AGAGGAGGAGGATAAGGAGAAGAAGCCCTCAGTGCCAAAAGATGA
TAAAGAGCAAGACGAGCACGACGCAGATGCACACCAGGGTGAAGT
CGGGCGGGGGGACGGAGGGCATGACGGCGCCGACTACCTAGACGA
AGGGAACGACGTGCTCTTGAAGCACCTGCATCGTCAGTGCGCCAT
CGTTTGCGACGCTCTGCAGGAGCGCAGCGAAGTGCCCCTCAGCGT
GGCGGAGGTCAGCCACGCCTACGAGCTCAGCCTCTTCTCCCCCCG
GGTGCCCCCCCGCCGCCGCGAAAACGGCACATGCGAGCCCAACCC
GCGCCTCAACTTCTACCCCGCCTTTGTGGTGCCCGAGGTCCTGGC
CACCTATCACATCTTCTTTCAAAATTGCAAGATCCCCCTCTCGTG
CCGCGCCAACCGTAGCCGCGCCGATAAGATGCTGGCCCTGCGCCA
GGGCGACCACATACCTGATATCGCCGCTTTGGAAGATGTGCCAAA
GATCTTCGAGGGTCTGGGTCGCAACGAGAAGCGGGCAGCAAACTC
TCTGCAACAGGAAAACAGCGAAAATGAGAGTCACACCGGGGTACT
GGTGGAGCTCGAGGGCGACAACGCCCGCCTGGCGGTGGTCAAGCG
CAGCATCGAGGTCACCCACTTTGCCTACCCCGCGCTCAACCTGCC
CCCCAAAGTCATGAACGCGGCCATGGACGGGCTGATCATGCGCCG
CGGCCGGCCCCTCGCTCCAGATGCAAACTTGCATGAGGAGACCGA
GGACGGCCAGCCCGTGGTCAGCGACGAGCAGCTGGCGCGCTGGCT
GGAGACCGCGGACCCCGCCGAACTGGAGGAGCGGCGCAAGATGAT
GATGGCCGCGGTGCTGGTCACCGTAGAGCTGGAGTGTCTGCAGCG
CTTCTTCGGCGACCCCGAGATGCAGAGAAAGGTCGAGGAGACCCT
GCACTACACCTTCCGCCAGGGCTACGTGCGCCAGGCTTGCAAGAT
CTCCAACGTGGAGCTCAGCAACCTGGTGTCCTACCTGGGCATCTT
GCATGAGAACCGCCTCGGGCAGAGCGTGCTGCACTCCACCCTGCG
CGGGGAGGCGCGCCGCGACTACGTGCGCGACTGCGTTTACCTCTT
CCTCTGCTACACCTGGCAGACGGCCATGGGGGTCTGGCAGCAGTG
CCTGGAGGAGCGCAACCTCAAGGAGCTGGAGAAGCTCCTGCAGCG
CGCGCTCAAAGACCTCTGGACGGGCTACAACGAGCGCTCGGTGGC
CGCCGCGCTGGCCGACCTCATCTTCCCCGAGCGCCTGCTCAAAAC
CCTCCAGCAGGGGCTGCCCGACTTCACCAGCCAAAGCATGTTGCA
AAACTTCAGGAACTTTATCCTGGAGCGTTCTGGCATCCTACCCGC
CACCTGCTGCGCCCTGCCCAGCGACTTTGTCCCCCTCGTGTACCG
CGAGTGCCCCCCGCCGCTGTGGGGTCACTGCTACCTGTTCCAACT
GGCCAACTACCTGTCCTACCACGCGGACCTCATGGAGGACTCCAG
CGGCGAGGGGCTCATGGAGTGCCACTGCCGCTGCAACCTCTGCAC
GCCCCACCGCTCCCTGGTCTGCAACACCCAACTGCTCAGCGAGAG
TCAGATTATCGGTACCTTCGAGCTACAGGGTCCGTCCTCCTCAGA
CGAGAAGTCCGCGGCTCCGGGGCTAAAACTCACTCCGGGGCTGTG
GACTTCCGCCTACCTGCGCAAATTTGTACCTGAAGACTACCACGC
CCACGAGATCAGGTTTTACGAAGACCAATCCCGCCCGCCCAAGGC
GGAGCTGACCGCCTGCGTCATCACCCAGGGCGAGATCCTAGGCCA
ATTGCAAGCCATCCAAAAAGCCCGCCAAGATTTTTTGCTGAGAAA
GGGTCGGGGGGTGTATCTGGACCCCCAGTCGGGTGAGGAGCTCAA
CCCGGTTCCCCCGCTGCCGCCGCCGCGGGACCTTGCTTCCCAGGA
TAAGCATCGCCATGGCTCCCAGAAAGAAGCAGCAGCGGCCGCCAC
TGCCGCCACCCCACATGCTGGAGGAAGAGGAGGAATACTGGGACA
GTCAGGCAGAGGAGGTTTCGGACGAGGAGGAGCCGGAGACGGAGA
TGGAAGAGTGGGAGGAGGACAGCTTAGACGAGGAGGCTTCCGAAG
CCGAAGAGGCAGACGCAACACCGTCACCCTCGGCCGCAGCCCCCT
CGCAGGCGCCCCCGAAGTCCGCTCCCAGCATCAGCAGCAACAGCA
GCGCTATAACCTCCGCTCCTCCACCGCCGCGACCCACGGCCGACC
GCAGACCCAACCGTAGATGGGACACCACCGGAACCGGGGCCGGTA
AGTCCTCCGGGAGAGGCAAGCAAGCGCAGCGCCAAGGCTACCGCT
CGTGGCGCGCTCACAAGAACGCCATAGTCGCTTGCTTGCAAGACT
GCGGGGGGAACATCTCCTTCGCCCGCCGCTTCCTGCTCTTCCACC
ACGGTGTGGCCTTCCCCCGTAACGTCCTGCATTACTACCGTCATC
TCTACAGCCCCTACTGCGGCGGCAGTGAGCCAGAGACGGTCGGCG
GCGGCGGCGGCGCCCGTTTCGGCGCCTAGGAAGACCCAGGGCAAG
ACTTCAGCCAAGAAACTCGCGGCGGCCGCGGCGAACGCGGTCGCG
GGGGCCCTGCGCCTGACGGTGAACGAACCCCTGTCGACCCGCGAA
CTGAGAAACCGAATCTTCCCCACTCTCTATGCCATCTTCCAGCAG
AGCAGAGGGCAGGATCAGGAACTGAAAGTAAAAAACAGGTCTCTG
CGCTCCCTCACCCGCAGCTGTCTGTATCACAAGAGCGAAGACCAG
CTTCGGCGCACGCTGGAGGACGCTGAGGCACTCTTCAGCAAATAC
TGCGCGCTCACTCTTAAGGACTAGCTCCGCGCCCTTCTCGAATTT
AGGCGGGAACGCCTACGTCATCGCAGCGCCGCCGTCATGAGCAAG
GACATTCCCACGCCATACATGTGGAGCTATCAGCCGCAGATGGGA
CTCGCGGCGGGCGCCTCCCAAGACTACTCCACCCGCATGAACTGG
CTCAGTGCCGGCCCACACATGATCTCACAGGTTAATGATATCCGC
ACCCATCGAAACCAAATATTGGTGGAGCAGGCGGCAATTACCACC
ACGCCCCGCAATAATCCCAACCCCAGGGAGTGGCCCGCGTCCCTG
GTGTATCAGGAAATTCCCGGCCCCACCACCGTACTACTTCCGCGT
GATTCCCAGGCCGAAGTCCAAATGACTAACTCAGGGGCGCAGCTC
GCGGGCGGCTGTCGTCACAGGGTGCGGCCTCCTCGCCAGGGTATA
ACTCACCTGGAGATCCGAGGCAGAGGTATTCAGCTCAACGACGAG
TCGGTGAGCTCCTCGCTCGGTCTAAGACCTGACGGGACCTTCCAG
ATAGCCGGAGCCGGCCGATCTTCCTTCACGCCCCGCCAGGCGTAC
CTGACTCTGCAGAGCTCGTCCTCGGCGCCGCGCTCGGGCGGCATC
GGGACTCTCCAGTTCGTGCAGGAGTTTGTGCCCTCGGTCTACTTC
AACCCCTTCTCGGGCTCTCCCGGTCGCTACCCGGACCAGTTCATC
TCGAACTTTGACGCCGCGAGGGACTCGGTGGACGGCTACGACTGA
ATGTCGGGTGGACCCGGTGCAGAGCAACTTCGCCTGAAGCACCTC
GACCACTGCCGCCGCCCTCAGTGCTTTGCCCGCTGTCAGACCGGT
GAGTTCCAGTACTTTTCCCTGCCCGACTCGCACCCGGACGGCCCG
GCGCACGGGGTGCGCTTTTTCATCCCGAGTCAGGTGCGCTCTACC
CTAATCAGGGAGTTTACCGCCCGTCCCCTACTGGCGGAGTTGGAA
AAGGGGCCTTCTATCCTAACCATTGCCTGCATCTGCTCTAACCCT
GGATTGCACCAAGATCTTTGCTGTCATTTGTGTGCTGAGTATAAT
AAAGGCTGAGATCAGAATCTACTCGGGCTCCTGTCGCCATCCTGT
CAACGCCACCGTCCAAGCCCGGCCCGATCAGCCCGAGGTGAACCT
CACCTGCGGTCTGCACCGGCGCCTGAGGAAATACCTAGCTTGGTA
CTACAACAGCACTCCCTTTGTGGTTTACAACAGCTTTGACCAGGA
CGGGGTCTCACTGAGGGATAACCTCTCGAACCTGAGCTACTCCAT
CAGGAAGAACAGCACCCTCGAGCTACTTCCTCCTTACCTGCCCGG
GACTTACCAGTGTGTCACCGGTCCCTGCACCCACACCCACCTGTT
GATCGTAAACGACTCTCTTCCGAGAACAGACCTCAATAACTCCTC
TTCGCAGTTCCCCAGAACAGGAGGTGAGCTCAGGAAACCCCGGGT
AAAGAAGGGTGGACAAGAGTTAACACTTGTGGGGTTTCTGGTGTA
TGTGACGCTGGTGGTGGCTCTTTTGATTAAGGCTTTTCCTTCCAT
GTCTGAACTCTCCCTCTTTTATGAACAACTCGACTAGTGCTAACG
GGACCCTACCCAACGAATCGGGATTGAATATCGGTAACCAGGTTG
CAGTTTCACTTTTGATTACCTTCATAGTCCTCTTCCTGCTAGTGC
TGTCGCTTCTGTGCCTGCGGATCGGGGGCTGCTGCATCCACGTTT
ATATCTGGTGCTGGCTGTTTAGAAGGTTCGGAGACCATCGCAGGT
AGAATAAACATGCTGCTGCTTACCCTCTTTGTCCTGGCGCTGGCC
GCCAGCTGCCAAGCCTTTTCCGAGGCTGACTTTATAGAGCCCCAG
TGTAACGTGACTTTTAAAGCCCATGCACAGCGTTGTCATACTATA
ATCAAATGTGCCACCGAACACGATGAATACCTTATCCAGTATAAA
GATAAATCACACAAAGTGGCACTTGTTGACATCTGGAAACCCGAA
GACCCTTTGGAATACAATGTGACCGTTTTCCAGGGTGACCTCTTC
AAAATTTACAATTACACTTTCCCATTTGACCAGATGTGTGACTTT
GTCATGTACATGGAAAAGCAGCACAAGCTGTGGCCTCCGACTCCC
CAGGGCTGTGTGGAAAATCCAGGCTCTTTCTGCATGATCTCTCTC
TGTGTAACTGTGCTGGCACTAATACTCACGCTTTTGTATATCAGA
TTTAAATCAAGGCAAAGCTTCATCGATGAAAAGAAAATGCCTTAA
ACGCTTTCACGCTTGATTGCTAACACCGGGTTTTTATCCGCAGAA
TGATTGGAATCACCCTACTAATCACCTCCCTCCTTGCGATTGCCC
ATGGGTTGGAACGAATCGAAGCCCCTGTGGGGGCCAATGTTACCC
TGGTGGGGCCTGTCGGCAATGCTACATTAATGTGGGAAAAATATA
CTAAAAATCAATGGGTCTCTTACTGCACTAACAAAAACAGCCACA
AGCCCAGAGCCATCTGCGATGGGCAAAATCTAACCTTGATTGATG
TTCAAATGCTGGATGCGGGCTACTATTATGGGCAGCTGGGTACAA
TGATTAATTACTGGAGACCCCACAAAGATTACATGCTCCACGTAG
TAAAGGGTCCCCTTAGCAGCCCACCCACTACCACCTCTACTACCC
CCACTACCACCACTACTCCCACCACCAGCACTGCCGCCCAGCCTC
CTCATAGCAGAACAACCACTTTTATCAATTCCAAGTCCCACTCCC
CCCACATTGCCGGCGGGCCCTCCGCCTCAGACTCCGAGACCACCG
AGATCTGCTTCTGCAAATGCTCTGACGCCTTTGCTGAGGATTTGG
AAGACCACGAGGAAGATGAGCATGACTTCGCAGATGCATGCCAGG
CATCAGAGGCAGAAGCGCTGCCGGTGGCCCTCAAACAGTATGCAG
ACCCCCACACCACCCCCAACCTTCCTCCACCTTCCCAGAAGCCAA
GTTTCCTGGGGGAAAATGAAACTCTGCCTCTCTCCATACTCGCTC
TGACATCTGTTGCTATGTTGACCGCTCTGCTGGTGCTTCTATGCT
CTATATGCTACCTGATCTGCTGCAGAAAGAAAAAATCTCACGGCC
ATGCTCACCAGCCCCTCATGCACTTCCCTTACCCTCCAGAGCTGG
GCGACCACAAACTTTAAGTCTGCAGTAACTATCTGCCCATCCCTT
GTCAGTCGACAGCGATGAGCCCCACTAATCTAACGGCCTCTGGAC
TTACAACATCGTCTCTTAATGAGACCACCGCTCCTCAAGACCTGT
ACGATGGTGTCTCCGCGCTGGTTAACCAGTGGGATCACCTGGGCA
TATGGTGGCTCCTCATAGGAGCAGTGACCCTGTGCCTAATCCTGG
TCTGGATCATCTGCTGCATCAAAAGCAGAAGACCCAGGCGGCGGC
CCATCTACAGGCCCTTTGTCATCACACCTGAAGATGATGATGACA
CCACTTCCAGGCTGCAGAGGCTAAAGCAGCTACTCTTCTCTTTTA
CAGCATGGTAAATTGAATCATGCCTCGCATTTTCATCTACTTGTC
TCTCCTTCCACTTTTTCTGGGCTCTTCTACATTGGCCGCTGTGTC
CCACATCGAGGTAGACTGCCTCACGCCCTTCACAGTCTACCTGCT
TTTCGGCTTTGTCATCTGCACCTTTGTCTGCAGCGTTATCACTGT
AGTGATCTGCTTCATACAGTGCATCGACTACGTCTGCGTGCGGGT
GGCTTACTTTAGACACCACCCCCAGTATCGCAACAGGGACATAGC
GGCTCTCCTAAGACTTGTTTAAAATCATGGCCAAATTAACTGTGA
TTGGTCTTCTGATCATCTGCTGCGTCCTAGCCGCGATTGGGACTC
AAGCTCCTACCACCACCAGCGCTCCCAGAAAGAGACATGTATCCT
GCAGCTTCAAGCGTCCCTGGAATATACCCCAATGCTTTACTGATG
AACCTGAAATCTCTTTGGCTTGGTACTTCAGCGTCACCGCCCTTC
TTATCTTCTGCAGTACGGTTATTGCCCTTGCCATCTACCCTTCCC
TTGACCTGGGCTGGAATGCTGTCAACTCTATGGAATATCCCACCT
TCCCAGAACCAGACCTGCCAGACCTGGTTGTTCTAAACGCGTTTC
CTCCTCCTGCTCCCGTTCAAAATCAGTTTCGCCCTCCGTCCCCCA
CGCCCACTGAGGTCAGCTACTTTAATCTAACAGGCGGAGATGACT
GAAAACCTAGACCTAGAAATGGACGGTCTCTGCAGCGAGCAACGC
ACACTAGAGAGGCGCCGGCAAAAAGAGCTCGAGCGTCTTAAACAA
GAGCTCCAAGACGCGGTGGCCATACACCAGTGCAAAAAAGGTGTC
TTCTGTCTGGTAAAACAGGCCACGCTCACCTATGAAAAAACAGGT
GACACCCACCGCCTAGGATACAAGCTGCCCACACAGCGCCAGAAG
TTCGCCCTCATGATAGGCGAACAACCCATCACCGTGACCCAGCAC
TCCGTGGAGACAGAAGGCTGCATACACGCTCCCTGTAGGGGCGCT
GACTGCCTCTACACCTTGATCAAAACCCTCTGCGGTCTCAGAGAC
CTCATCCCTTTTAATTAATCATAACTGTAATCAATAAAAAATCAC
TTACTTGAAATCTGATAGCAAGCCTCTGTCCAATTTTTTCAGCAA
CACTTCCTTCCCCTCCTCCCAACTCTGGTACTCTAGGCGCCTCCT
AGCTGCAAACTTCCTCCACAGTCTGAAGGGAATGTCAGATTCCTC
CTCCTGTCCCTCCGCACCCACGATCTTCATGTTGTTGCAGATGAA
ACGCGCGAGATCGTCTGACGAGACCTTCAACCCCGTGTACCCCTA
CGATACCGAGATCGCTCCGACTTCTGTCCCTTTCCTTACCCCTCC
CTTTGTGTCATCCGCAGGAATGCAAGAAAATCCAGCTGGGGTGCT
GTCCCTGCACTTGTCAGAGCCCCTTACCACCCACAATGGGGCCCT
GACTCTAAAAATGGGGGGCGGCCTGACCCTGGACAAGGAAGGGAA
TCTCACTTCCCAAAACATCACCAGTGTCGATCCCCCTCTCAAAAA
AAGCAAGAACAACATCAGCCTTCAGACCGCCGCACCCCTCGCCGT
CAGCTCCGGGGCCCTAACACTTTTTGCCACTCCCCCCCTAGCGGT
CAGTGGTGACAACCTTACTGTGCAGTCTCAGGCCCCTCTCACTTT
GGAAGACTCAAAACTAACTCTGGCCACCAAAGGACCCCTAACTGT
GTCCGAAGGCAAACTTGTCCTAGAAACAGAGGCTCCCCTGCATGC
AAGTGACAGCAGCAGCCTGGGCCTTAGCGTTACGGCCCCACTTAG
CATTAACAATGACAGCCTAGGACTAGACATGCAAGCGCCCATTAG
CTCTCGAGATGGAAAACTGGCTCTAACAGTGGCGGCCCCCCTAAC
TGTGGTCGAGGGTATCAATGCTTTGGCAGTAGCCACAGGTAAGGG
TATTGGGCTAAATGAAACCAACACACACCTGCAGGCAAAACTGGT
CGCACCCCTAGGCTTTGATACCAACGGCAACATTAAGCTAAGCGT
TGCAGGAGGCATGAGGCTAAACAATAACACACTGATACTAGATGT
AAACTACCCATTTGAGGCTCAAGGCCAACTGAGCCTAAGAGTGGG
CTCGGGCCCACTATATGTAGATTCTAGTAGTCATAACCTAACCAT
TAGATGCCTTAGGGGATTGTATATAACATCTTCTAACAACCAAAA
CGGTCTAGAAGCCAACATTAAACTAACAAGAGGCCTTGTGTATGA
CGGAAATGCCATAGCAGTTAATGTTGGCAAAGGGCTGGAATACAG
CCCTACTGACACAACAGAAAAACCTATACAGACTAAAATAGGTCT
AGGCATGGAGTATGATACCGAGGGAGCCATGATGACAAAACTAGG
CTCTGGACTAAGCTTTGACAATTCAGGAGCCATTGTAGTGGGAAA
CAAAAATGATGACAGGCTTACTTTGTGGACCACACCGGACCCATC
GCCCAACTGTCAGATCTACTCTGAAAAAGATGCTAAACTAACCTT
GGTACTGACTAAATGTGGCAGTCAGGTTGTAGGCACAGTATCTAT
TGCCGCTCTTAAAGGTAGCCTCGTGCCAATCACTAGTGCAATCAG
TGTGGTTCAGGTATACCTAAGGTTTGATGAAAATGGGGTACTAAT
GAGTAACTCTTCACTTAATGGCGAATACTGGAATTTTAGAAACGG
AGACTCAACTAATGGCACACCATATACAAACGCAGTGGGTTTCAT
GCCTAATCTACTGGCCTATCCTAAAGGTCAAACTACAACTGCAAA
AAGTAACATTGTCAGCCAGGTCTACATGAATGGGGACGATACTAA
ACCCATGACATTTACAATCAACTTCAATGGCCTTAGTGAAACAGG
GGATACCCCTGTTAGTAAATATTCCATGACATTCTCATGGAGGTG
GCCAAATGGAAGCTACATAGGGCACAATTTTGTAACAAACTCCTT
TACCTTCTCCTACATCGCCCAAGAATAAAGAAAGCACAGAGATGC
TTGTTTTTGATTTCAAAATTGTGTGCTTTTATTTATTTTCAGCTT
ACAGTATTTCCAGTAGTCATTCAAATAAAGCTTAATCAAACTGCA
TGAGAACCCTTCCACATAGCTTAAATTAGCACCAGTGCAAATGGA
GAAAACAATTGACGGCCGCCATAGAGCCCACCGCATCCCCAGCAT
GCCTGCTATTGTCTTCCCAATCCTCCCCCTTGCTGTCCTGCCCCA
CCCCACCCCCCAGAATAGAATGACACCTACTCAGACAATGCGATG
CAATTTCCTCATTTTATTAGGAAAGGACAGTGGGAGTGGCACCTT
CCAGGGTCAAGGAAGGCACGGGGGAGGGGCAAACAACAGATGGCT
GGCAACTAGAAGGCACAGGGTACCAACGCGTCGTAACTATAACGG
TCCTAAGGTAGCGAAAAGCACTCTCACAGCACCAGCACTAATCAG
AGTGTGAAGAGGGCCAAGTGCCGAACGAGTATATATAGGAATAAA
AAATGACGTAAATGTGTAAAGGTCAGAAAACGCCCAGAAAAATAC
ACAGACCAACGCCCGAAACGAAAACCCGCGAAAAAATACCCAGAA
CTTCCTCAACAACCGCCACTTCCGCTTTCTCACGGTACGTCACTT
CCGCAAGAAAAGCAAAACTACATTTCCCACATGTGTAAAAACGAA
ACCCCGCCCCTTGTAACCGCCCACAACTTACATCATCAAAACGTA
AACTCCTACGTCACCCGCCCCGCCTCTCCCCGCCCACCTCATTAT
CATATTGGCCACAATCCAAAATAAGGTATATTATTGATGATG

VARIANTS OF SEQ ID NO: 68

SEQ ID NO: 134
MADDSALHKKYPFLNLLHTPPHRPPPLCPQAPRKTQCKRRLGNEHEESNSPLATPCTA
EIYAYAYKNLKVVQCTETDIREVQQLLLGTLNIVCPICAPKTTAEIYSYAYKQLKVLQ
CKRRLGNEHEESNSPLATPCVWPTLDPWTVETTTSSLTITTSTKDGTTVTVQLRTAEIY
AYAYKNLKVVMHGRHVTLKDIVLDLQPPDPVGLHCYEQLVDSSEDEVDEVDGQDSQ
PLKQHFQIVTCCHCYEQLEDSSEDEVDMESANASTSATTIDQLCKTFNLSMHTLQINC
VFCKNALTTAEIYSYAYKHLKVLFRGGYPYAACACCLEFHGKINQYAHFDYAGYATT
VEEETKQDILDVLIRCYLCHKPQCEVEKVKHILTKARFIKLNCTRKGRCLHCWTTCME
DMLPMEAIAKRLDACAEQLLELAEENSTDLHKHVLHWKCMRHASVLLYKAKQMGL
SHIGMQVVPPLKVSEAKGHNAIEMQMHLESLLRTEYSMEPWTLQETSYAMWQTPPK
RCFKKRGKTVEVKFDGCANNTMDYVVWTDVYVQDNDTWVKVHSMVDAKGIYYTC
GQFKTYYVNFVKEAEKYGSTKHWEVCYGSTVICSPASVSSTTQEVSIPESTTYTPAQTS
TLVSSSTKEDAVQTPPRKRARGVQQSPCNALCVAHIGPVDSGNHNLITNNHDQHQRR
NNSNSSATPIVQFQGESNCLKCFRYRLNDRHRHLFDLISSTWHWASSKAPHKHAIVTV
TYDSEEQRQQFLDVVKIPPTISHKLGFMSLHLLHCYEQLEDSSEDEVD
SEQ ID NO: 135
MESANASTSATTIDQLCKTFNLSMHTLQINCVFCKNALTTAEIYSYAYKHLKVLFRGG
YPYAACACCLEFHGKINQYAHFDYAGYATTVEEETKQDILDVLIRCYLCHKPQCEVE
KVKHILTKARFIKLNCTRKGRCLHCWTTCMEDMLPHCYEQLEDSSEDEVDMEAIAKR
LDACAEQLLELAEENSTDLHKHVLHWKCMRHASVLLYKAKQMGLSHIGMQVVPPL
KVSEAKGHNAIEMQMHLESLLRTEYSMEPWTLQETSYAMWQTPPKRCFKKRGKTVE
VKFDGCANNTMDYVVWTDVYVQDNDTWVKVHSMVDAKGIYYTCGQFKTYYVNFV
KEAEKYGSTKHWEVCYGSTVICSPASVSSTTQEVSIPESTTYTPAQTSTLVSSSTKEDA
VQTPPRKRARGVQQSPCNALCVAHIGPVDSGNHNLITNNHDQHQRRNNSNSSATPIV
QFQGESNCLKCFRYRLNDRHRHLFDLISSTWHWASSKAPHKHAIVTVTYDSEEQRQQ
FLDVVKIPPTISHKLGFMSLHLLHCYEQLEDSSEDEVDMADDSALHKKYPFLNLLHTP
PHRPPPLCPQAPRKTQCKRRLGNEHEESNSPLATPCTAEIYAYAYKNLKVVQCTETDI
REVQQLLLGTLNIVCPICAPKTTAEIYSYAYKQLKVLQCKRRLGNEHEESNSPLATPCV
WPTLDPWTVETTTSSLTITTSTKDGTTVTVQLRTAEIYAYAYKNLKVVMHGRHVTLK
DIVLDLQPPDPVGLHCYEQLVDSSEDEVDEVDGQDSQPLKQHFQIVTCC
SEQ ID NO: 136
MHGRHVTLKDIVLDLQPPDPVGLHCYEQLVDSSEDEVDEVDGQDSQPLKQHFQIVTC
CHCYEQLEDSSEDEVDMESANASTSATTIDQLCKTFNLSMHTLQINCVFCKNALTTAE
IYSYAYKHLKVLFRGGYPYAACACCLEFHGKINQYAHFDYAGYATTVEEETKQDILD
VLIRCYLCHKPQCEVEKVKHILTKARFIKLNCTRKGRCLHCWTTCMEDMLPMADDSA
LHKKYPFLNLLHTPPHRPPPLCPQAPRKTQCKRRLGNEHEESNSPLATPCTAEIYAYAY
KNLKVVQCTETDIREVQQLLLGTLNIVCPICAPKTTAEIYSYAYKQLKVLQCKRRLGN
EHEESNSPLATPCVWPTLDPWTVETTTSSLTITTSTKDGTTVTVQLRTAEIYAYAYKNL
KVVMEAIAKRLDACAEQLLELAEENSTDLHKHVLHWKCMRHASVLLYKAKQMGLS
HIGMQVVPPLKVSEAKGHNAIEMQMHLESLLRTEYSMEPWTLQETSYAMWQTPPKR
CFKKRGKTVEVKFDGCANNTMDYVVWTDVYVQDNDTWVKVHSMVDAKGIYYTCG
QFKTYYVNFVKEAEKYGSTKHWEVCYGSTVICSPASVSSTTQEVSIPESTTYTPAQTST
LVSSSTKEDAVQTPPRKRARGVQQSPCNALCVAHIGPVDSGNHNLITNNHDQHQRRN
NSNSSATPIVQFQGESNCLKCFRYRLNDRHRHLFDLISSTWHWASSKAPHKHAIVTVT
YDSEEQRQQFLDVVKIPPTISHKLGFMSLHLLHCYEQLEDSSEDEVD
SEQ ID NO: 137
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYAYKNLKVVQ
CTETDIREVQQLLLGTLNIVCPICAPKTTAEIYSYAYKQLKVLQCKRRLGNEHEESNSP
LATPCVWPTLDPWTVETTTSSLTITTSTKDGTTVTVQLRTAEIYAYAYKNLKVVMHG
RHVTLKDIVLDLQPPDPVGLHCYEQLVDSSEDEVDEVDGQDSQPLKQHFQIVTCCME
SANASTSATTIDQLCKTFNLSMHTLQINCVFCKNALTTAEIYSYAYKHLKVLFRGGYP
YAACACCLEFHGKINQYAHFDYAGYATTVEEETKQDILDVLIRCYLCHKPQCEVEKV
KHILTKARFIKLNCTRKGRCLHCWTTCMEDMLP
SEQ ID NO: 138
MADDSALHKKYPFLNLLHTPPHRPPPLCPQAPRKTQCKRRLGNEHEESNSPLATPCTA
EIYAYAYKNLKVVTAEIYSYAYKQLKVLQCKRRLGNEHEESNSPLATPCVWPTLDPW
TVETTTSSLTITTSTKDGTTVTVQLRTAEIYAYAYKNLKVVMHGRHVTLKDIVLDLQP
PDPVGLHCYEQLVDSSEDEVDEVDGQDSQPLKQHFQIVTCCHCYEQLEDSSEDEVDM
ESANASTSATTIDQLCKTFNLSMHTLQINCVFCKNALTTAEIYSYAYKHLKVLFRGGY
PYAACACCLEFHGKINQYAHFDYAGYATTVEEETKQDILDVLIRCYLCHKPQCEVEK
VKHILTKARFIKLNCTRKGRCLHCWTTCMEDMLPMEAIAKRLDACAEQLLELAEENS
TDLHKHVLHWKCMRHASVLLYKAKQMGLSHIGMQVVPPLKVSEAKGHNAIEMQMH
LESLLRTEYSMEPWTLQETSYAMWQTPPKRCFKKRGKTVEVKFDGCANNTMDYVV
WTDVYVQDNDTWVKVHSMVDAKGIYYTCGQFKTYYVNFVKEAEKYGSTKHWEVC
YGSTVICSPASVSSTTQEVSIPESTTYTPAQTSTLVSSSTKEDAVQTPPRKRARGVQQSP
CNALCVAHIGPVDSGNHNLITNNHDQHQRRNNSNSSATPIVQFQGESNCLKCFRYRLN
DRHRHLFDLISSTWHWASSKAPHKHAIVTVTYDSEEQRQQFLDVVKIPPTISHKLGFM
SLHLLHCYEQLEDSSEDEVD
SEQ ID NO: 139
MHGRHVTLKDIVLDLQPPDPVGLHCYEQLVDSSEDEVDEVDGQDSQPLKQHFQIVTC
CHCYEQLEDSSEDEVDMESANASTSATTIDQLCKTFNLSMHTLQINCVFCKNALTTAE
IYSYAYKHLKVLFRGGYPYAACACCLEFHGKINQYAHFDYAGYATTVEEETKQDILD
VLIRCYLCHKPQCEVEKVKHILTKARFIKLNCTRKGRCLHCWTTCMEDMLPTAEIYA
YAYKNLKVVQCTETDIREVQQLLLGTLNIVCPICAPKTTAEIYSYAYKQLKVLTAEIYA
YAYKNLKVVMEAIAKRLDACAEQLLELAEENSTDLHKHVLHWKCMRHASVLLYKA
KQMGLSHIGMQVVPPLKVSEAKGHNAIEMQMHLESLLRTEYSMEPWTLQETSYAMW
QTPPKRCFKKRGKTVEVKFDGCANNTMDYVVWTDVYVQDNDTWVKVHSMVDAKG
IYYTCGQFKTYYVNFVKEAEKYGSTKHWEVCYGSTVICSPASVSSTTQEVSIPESTTYT
PAQTSTLVSSSTKEDAVQTPPRKRARGVQQSPCNALCVAHIGPVDSGNHNLITNNHDQ
HQRRNNSNSSATPIVQFQGESNCLKCFRYRLNDRHRHLFDLISSTWHWASSKAPHKH
AIVTVTYDSEEQRQQFLDVVKIPPTISHKLGFMSLHLLHCYEQLEDSSEDEVD
SEQ ID NO: 140
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYAYKNLKVVQ
CTETDIREVQQLLLGTLNIVCPICAPKTTAEIYSYAYKQLKVLQCKRRLGNEHEESNSP
LATPCVWPTLDPWTVETTTSSLTITTSTKDGTTVTVQLRTAEIYAYAYKNLKVVMHG
RHVTLKDIVLDLQPPDPVGLHCYEQLVDSSEDEVDEVDGQDSQPLKQHFQIVTCCHC
YEQLEDSSEDEVDMESANASTSATTIDQLCKTFNLSMHTLQINCVFCKNALTTAEIYS
YAYKHLKVLFRGGYPYAACACCLEFHGKINQYAHFDYAGYATTVEEETKQDILDVLI
RCYLCHKPQCEVEKVKHILTKARFIKLNCTRKGRCLHCWTTCMEDMLPTAEIYAYAY
KNLKVV
SEQ ID NO: 141
QCTETDIREVQQLLLGTLNIVCPICAPKTMHGRHVTLKDIVLDLQPPDPVGLHCYEQL
VDSSEDEVDEVDGQDSQPLKQHFQIVTCCHCYEQLEDSSEDEVDMESANASTSATTID
QLCKTFNLSMHTLQINCVFCKNALTTAEIYSYAYKHLKVLFRGGYPYAACACCLEFH
GKINQYAHFDYAGYATTVEEETKQDILDVLIRCYLCHKPQCEVEKVKHILTKARFIKL
NCTRKGRCLHCWTTCMEDMLPMADDSALHKKYPFLNLLHTPPHRPPPLCPQAPRKT
QCKRRLGNEHEESNSPLATPCTAEIYAYAYKNLKVVQCTETDIREVQQLLLGTLNIVC
PICAPKTTAEIYSYAYKQLKVLQCKRRLGNEHEESNSPLATPCVWPTLDPWTVETTTS
SLTITTSTKDGTTVTVQLRTAEIYAYAYKNLKVVMEAIAKRLDACAEQLLELAEENST
DLHKHVLHWKCMRHASVLLYKAKQMGLSHIGMQVVPPLKVSEAKGHNAIEMQMHL
ESLLRTEYSMEPWTLQETSYAMWQTPPKRCFKKRGKTVEVKFDGCANNTMDYVVW
TDVYVQDNDTWVKVHSMVDAKGIYYTCGQFKTYYVNFVKEAEKYGSTKHWEVCY
GSTVICSPASVSSTTQEVSIPESTTYTPAQTSTLVSSSTKEDAVQTPPRKRARGVQQSPC
NALCVAHIGPVDSGNHNLITNNHDQHQRRNNSNSSATPIVQFQGESNCLKCFRYRLN
DRHRHLFDLISSTWHWASSKAPHKHAIVTVTYDSEEQRQQFLDVVKIPPTISHKLGFM
SLHLLHCYEQLEDSSEDEVD
SEQ ID NO: 142
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYAYKNLKVVQ
CTETDIREVQQLLLGTLNIVCPICAPKTMADDSALHKKYPFLNLLHTPPHRPPPLCPQA
PRKTQCKRRLGNEHEESNSPLATPCTAEIYSYAYKQLKVLQCKRRLGNEHEESNSPLA
TPCVWPTLDPWTVETTTSSLTITTSTKDGTTVTVQLRTAEIYAYAYKNLKVVMHGRH
VTLKDIVLDLQPPDPVGLHCYEQLVDSSEDEVDEVDGQDSQPLKQHFQIVTCCHCYE
QLEDSSEDEVDMHGRHVTLKDIVLDLQPPDPVGLHCYEQLVDSSEDEVDEVDGQDSQ
PLKQHFQIVTCCMESANASTSATTIDQLCKTFNLSMHTLQINCVFCKNALTTAEIYSYA
YKHLKVLFRGGYPYAACACCLEFHGKINQYAHFDYAGYATTVEEETKQDILDVLIRC
YLCHKPQCEVEKVKHILTKARFIKLNCTRKGRCLHCWTTCMEDMLP
SEQ ID NO: 143
MESISKRLDSCSEQLLELSEENSTDLHKHVLHWKCMRHSSVLLYKSKQMGLSHIGMQ
VVPPLKVSESKGHNSIEMQMHLESLLRTEYSMEPWTLQETSYSMWQTPPKRCFKKRG
KTVEVKFDGCSNNTMDYVVWTDVYVQDNDTWVKVHSMVDSKGIYYTCGQFKTYY
VNFVKESEKYGSTKHWEVCYGSTVICSPSSVSSTTQEVSIPESTTYTPSQTSTLVSSSTK
EDSVQTPPRKRSRGVQQSPCNSLCVSHIGPVDSGNHNLITNNHDQHQRRNNSNSSSTPI
VQFQGESNCLKCFRYRLNDRHRHLFDLISSTWHWSSSKSPHKHSIVTVTYDSEEQRQQ
FLDVVKIPPTISHKLGFMSLHLLHCYEQLEDSSEDEVDMADDSALHKKYPFLNLLHTP
PHRPPPLCPQAPRKTQCKRRLGNEHEESNSPLATPCTAEIYAYAYKNLKVVQCTETDI
REVQQLLLGTLNIVCPICAPKTTAEIYSYAYKQLKVLQCKRRLGNEHEESNSPLATPCV
WPTLDPWTVETTTSSLTITTSTKDGTTVTVQLRTAEIYAYAYKNLKVVMHGRHVTLK
DIVLDLQPPDPVGLHCYEQLVDSSEDEVDEVDGQDSQPLKQHFQIVTCCHCYEQLEDS
SEDEVDMESANASTSATTIDQLCKTFNLSMHTLQINCVFCKNALTTAEIYSYAYKHLK
VLFRGGYPYAACACCLEFHGKINQYAHFDYAGYATTVEEETKQDILDVLIRCYLCHK
PQCEVEKVKHILTKARFIKLNCTRKGRCLHCWTTCMEDMLP
SEQ ID NO: 144
MESISKRLDSCSEQLLELSEENSTDLHKHVLHWKCMRHSSVLLYKSKQMGLSHIGMQ
VVPPLKVSESKGHNSIEMQMHLESLLRTEYSMEPWTLQETSYSMWQTPPKRCFKKRG
KTVEVKFDGCSNNTMDYVVWTDVYVQDNDTWVKVHSMVDSKGIYYTCGQFKTYY
VNFVKESEKYGSTKHWEVCYGSTVICSPSSVSSTTQEVSIPESTTYTPSQTSTLVSSSTK
EDSVQTPPRKRSRGVQQSPCNSLCVSHIGPVDSGNHNLITNNHDQHQRRNNSNSSSTPI
VQFQGESNCLKCFRYRLNDRHRHLFDLISSTWHWSSSKSPHKHSIVTVTYDSEEQRQQ
FLDVVKIPPTISHKLGFMSLHLLHCYEQIEDSSEDEVDMADDSALHKKYPFLNLLHTPP
HRPPPLCPQAPRKTQCKRRLGNEHEESNSPLATPCTAEIYAYAYKNLKVVQCTETDIR
EVQQLLLGTLNIVCPICAPKTTAEIYSYAYKQLKVLQCKRRLGNEHEESNSPLATPCV
WPTLDPWTVETTTSSLTITTSTKDGTTVTVQLRTAEIYAYAYKNLKVVMHGRHVTLK
DIVLDLQPPDPVGLHCYEQLVDSSEDEVDEVDGQDSQPLKQHFQIVTCCHCYEQLEDS
SEDEVDMESANASTSATTIDQLCKTFNLSMHTLQINCVFCKNALTTAEIYSYAYKHLK
VLFRGGYPYAACACCLEFHGKINQYAHFDYAGYATTVEEETKQDILDVLIRCYLCHK
PQCEVEKVKHILTKARFIKLNCTRKGRCLHCWTTCMEDMLP
SEQ ID NO: 145
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCCYEQLEDSSEIDEVDGMADDSALHKKY
PFLNLLHTPPHRPPPLCPQAPRKTQCKRRLGNEHEESNSPLATPCTAEIYAYAYKNLKV
VQCTETDIREVQQLLLGTLNIVCPICAPKTTAEIYSYAYKQLKVLQCKRRLGNEHEESN
SPLATPCVWPTLDPWTVETTTSSLTITTSTKDGTTVTVQLRTAEIYAYAYKNLKVVMH
GRHVTLKDIVLDLQPPDPVGLHCYEQLVDSSEDEVDEVDGQDSQPLKQHFQIVTCCH
CYEQLEDSSEDEVDMESANASTSATTIDQLCKTFNLSMHTLQINCVFCKNALTTAEIYS
YAYKHLKVLFRGGYPYAACACCLEFHGKINQYAHFDYAGYATTVEEETKQDILDVLI
RCYLCHKPQCEVEKVKHILTKARFIKLNCTRKGRCLHCWTTCMEDMLP
SEQ ID NO: 146
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYVQTNHGLIAY
KNLKVVQCTETDIREVQQLLLGTLNIVCPICAPKTTAEIYSYAYKQLKVLQCKRRLGN
EHEESNSPLATPCVWPTLDPWTVETTTSSLTITTSTKDGTTVTVQLRTAEIYAYAYKNL
KVVMHGRHVTLKDIVLDLQPPDPVGLHCYEQLVDSSEDEVDEVDGQDSQPLKQHFQI
VTCCHCYEQLEDSSEDEVDMESANASTSATTIDQLCKTFNLSMHTLQINCVFCKNALT
TAEIYSYAYKHLKVLFRGGYPYAACACCLEFHGKINQYAHFDYAGYATTVEEETKQD
ILDVLIRCYLCHKPQCEVEKVKHILTKARFIKLNCTRKGRCLHCWTTCMEDMLP
SEQ ID NO: 147
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYKNLKVVQCT
ETDIREVQQLLLGTLNIVCPICAPKTTAEIYSYAYKQLKVLQCKRRLGNEHEESNSPLA
TPCVWPTLDPWTVETTTSSLTITTSTKDGTTVTVQLRTAEIYAYAYKNLKVVMHGRH
VTLKDIVLDLQPPDPVGLHCYEQLVDSSEDEVDEVDGQDSQPLKQHFQIVTCCHCYE
QLEDSSEDEVDMESANASTSATTIDQLCKTFNLSMHTLQINCVFCKNALTTAEIYSYA
YKHLKVLFRGGYPYAACACCLEFHGKINQYAHFDYAGYATTVEEETKQDILDVLIRC
YLCHKPQCEVEKVKHILTKARFIKLNCTRKGRCLHCWTTCMEDMLP
SEQ ID NO: 148
MEAIAKRLDACAEQLLELAEENSTDLHKHVLHWKCMRHASVLLYKAKQMGLSHIG
MQVVPPLKVSEAKGHNAIEMQMHLESLLRTEYSMEPWTLQETSYAMWQTPPKRCFK
KRGKTVEVKFDGCANNTMDYVVWTDVYVQDNDTWVKVHSMVDAKGIYYTCGQFK
TYYVNFVKEAEKYGSTKHWEVCYGSTVICSPASVSSTTQEVSIPESTTYTPAQTSTLVS
SSTKEDAVQTPPRKRARGVQQSPCNALCVAHIGPVDSGNHNLITNNHDQHQRRNNSN
SSATPIVQFQGESNCLKCFRYRLNDRHRHLFDLISSTWHWASSKAPHKHAIVTVTYDS
EEQRQQFLDVVKIPPTISHKLGFMSLHLLHCYEQLEDSSEDEVDMADDSALHKKYPFL
NLLHTPPHRPPPLCPQAPRKTQCKRRLGNEHEESNSPLATPCTAEIYAYAYKNLKVVQ
CTETDIREVQQLLLGTLNIVCPICAPKTTAEIYSYAYKQLKVLQCKRRLNEHEESNSPL
ATPCVWPDPWTVETTTSSLTITSTKDGTTVTVQLRTAEIYAYAYKNLKVVMHGRHVT
LKDIVLDLQPPDPVGLHCYEQLVDSSEDEVDEVDGQDSQPLKQHFQIVTCCHCYEQLE
DSSEDEVDMESANASTSATTIDQLCKTFNLSMHTLQINCVFCKNALTTAEIYSYAYKH
LKVLFRGGYPYAACACCLEFHGKINQYAHFDYAGYATTVEEETKQDILDVLIRCYLC
HKPQCEVEKVKHILTKARFIKLNCTRKGRCLHCWTTCMEDMLP

| Antigen | Size | | SEQ ID |
|---|---|---|---|
| region | (aa) | Amino acid sequence | NO: |
| HPV E2- | 368 | MEAIAKRLDACAEQLLELAEENSTDLHKHVLHWKCMR | 154 |
| Variant | | HASVLLYKAKQMGLSHIGMQVVPPLKVSEAKGHNAIE | |
| A | | MQMHLESLLRTEYSMEPWTLQETSYGMWQTPPKRCFK | |
| | | KRGKTVEVKFDGCANNTMDYVVWTDVYVQDNDTWV | |
| | | KVHSMVDAKGIYYTCGQFKTYYVNFVKEAEKYGSTKH | |
| | | WEVCYGSTVICSPGSVSSTTQEVSIPESTTYTPAQTSTLV | |
| | | SSSTKEDAVQTPPRKRARGVQQSPCNALCVAHIGPVDS | |
| | | GNHNLITNNHDQHQRRNNSNSSGTPIVQFQGESNCLKC | |
| | | FRYRLNDRHRHLFDLISSTWHWASSKAPHKHAIVTVTY | |
| | | DSEEQRQQFLDVVKIPPTISHKLGFMSLHLL | |
| HPV E2- | 368 | MEAIAKRLSACAEQLLELAEENSTDLHKHVLHWKCMR | 155 |
| Variant | | HASVLLYKAKQMGLSHIGMQVVPPLKVSEAKGHNAIE | |
| B | | MQMHLESLLRTEYSMEPWTLQETSYAMWQTPPKRCFK | |
| | | KRGKTVEVKFSGCANNTMDYVVWTDVYVQDNDTWV | |
| | | KVHSMVDAKGIYYTCGQFKTYYVNFVKEAEKYGSTKH | |
| | | WEVCYGSTVICSPASVSSTTQEVSIPESTTYTPAQTSTLV | |
| | | SSSTKESAVQTPPRKRARGVQQSPCNALCVAHIGPVDS | |
| | | GNHNLITNNHDQHQRRNNSNSSATPIVQFQGESNCLKC | |
| | | FRYRLNDRHRHLFDLISSTWHWASSKAPHKHAIVTVTY | |
| | | DSEEQRQQFLDVVKIPPTISHKLGFMSLHLL | |
| HPV E2 | 368 | MEAIAKRLDACAEQLLELAEENSTDMHKHVMHWKCM | 156 |
| Variant | | RHASVLLYKAKQMGLSHIGMQVVPPLKVSEAKGHNAI | |
| C | | EMQMHLESLLRTEYSMEPWTLQETSYAMWQTPPKRCF | |
| | | KKRGKTVEVKFDGCANNTMDYVVWTDVYVQDNDTW | |
| | | VKVHSMVDAKGIYYTCGQFKTYYVNFVKEAEKYGSTK | |
| | | HWEVCYGSTVICSPASVSSTTQEVSIPESTTYTPAQTSTL | |
| | | VSSSTKEDAVQTPPRKRARGVQQSPCNALCVAHIGPVD | |
| | | SGNHNLITNNHDQHQRRNNSNSSATPIVQFQGESNCLK | |
| | | CFRYRLNDRHRHLFDLISSTWHWASSKAPHKHAIVTVT | |
| | | YDSEEQRQQFLDVVKIPPTISHKLGFMSLHLL | |
| HPV E2- | 368 | MEAIAKRLDACAEQLLELAEENSTDLHKHVLHWKCMR | 157 |
| Variant | | HASVLLYKAKQMGLSHIGMQTTPPLKVSEAKGHNAIE | |
| D | | MQMHLESLLRTEYSMEPWTLQETSYAMWQTPPKRCFK | |
| | | KRGKTVEVKFDGCANNTMDYVVWTDVYVQDNDTWV | |
| | | KVHSMVDAKGIYYTCGQFKTYYVNFVKEAEKYGSTKH | |
| | | WEVCYGSTVICSPASTSSTTQEVSIPESTTYTPAQTSTLV | |
| | | SSSTKEDAVQTPPRKRARGVQQSPCNALCVAHIGPVDS | |
| | | GNHNLITNNHDQHQRRNNSNSSATPIVQFQGESNCLKC | |
| | | FRYRLNDRHRHLFDLISSTWHWASSKAPHKHAIVTVTY | |
| | | DSEEQRQQFLDTTKIPPTISHKLGFMSLHLL | |
| HPV E2- | 368 | MEAIAKRLDSCSEQLLELAEENSTDLHKHVLHWKCMR | 158 |
| Variant | | HASVLLYKAKQMGLSHIGMQVVPPLKVSEAKGHNAIE | |
| E | | MQMHLESLLRTEYSMEPWTLQETSYAMWQTPPKRCFK | |
| | | KRGKTVEVKFDGCANNTMDYVVWTDVYVQDNDTWV | |
| | | KVHSMVDAKAIYYTCAQFKTYYVNFVKEAEKYGSTKH | |
| | | WEVCYGSTVICSPASVSSTTQEVSIPESTTYTPAQTSTLV | |
| | | SSSTKEDAVQTPPRKRARGVQQSPCNALCVAHIGPVDS | |
| | | GNHNLITNNHDQHQRRNNSNSSATPIVQFQGESNCLKC | |
| | | FRYRLNDRHRHLFDLISSTWHWASSKAPHKHAIVTVTY | |
| | | DSEEQRQQFLDVVKIPPTISHKLGFMSLHLL | |
| HPV E2- | 368 | MEAIAKRLDACAEQLLELAEENSTDLHKHVLHWKCMR | 159 |
| Variant | | HASVLLYDADQMGLSHIGMQVVPPLKVSEAKGHNAIE | |
| F | | MQMHLESLLRTEYSMEPWTLQETSYAMWQTPPKRCFK | |
| | | KRGKTVEVKFDGCANNTMDYVVWTDVYVQSNSTWV | |
| | | KVHSMVDAKGIYYTCGQFKTYYVNFVKEAEKYGSTKH | |
| | | WEVCYGSTVICSPASVSSTTQEVSIPESTTYTPAQTSTLV | |
| | | SSSTKEDAVQTPPRKRARGVDDSPCNALCVAHIGPVDS | |
| | | GNHNLITNNHDQHQKKNNSNSSATPIVQFQGESNCLKC | |
| | | FRYRLNDRHRHLFDLISSTWHWASSKAPHKHAIVAVAY | |
| | | DSEEQRQQFLDVVKIPPTISHKLGFMSLHLL | |
| HPV E2- | 368 | MEAIAKRLDACAEQVVELAEENSTDLHKHVLHWKCM | 160 |
| Variant | | RHASVLLYKAKQMGLSHIGMQLLPPLKVSEAKGHNAIE | |
| G | | MQMHLESMMRTEYSMEPWTLQETSYAMWQTPPKRCF | |
| | | KKRGKTVEVKFDGCANNTMDYAAWTDVYVQDNDTW | |
| | | LKLHSMVDAKGIYYTCGQFKTYYVNFVKEAEKYGSTK | |
| | | HWEVCYGSTVICSPASVGGTTQEVSIPESAAYTPAQTST | |
| | | LVSSSTKEDAVQTPPRKRARGVQQSPCNALCVAHIGPV | |
| | | DSGGHGLITNNHDQHQRRNNSNSSATPIVQFQGESNCL | |
| | | KCFRYRLNDRHRHLFDLIAATWHWASSKAPHKHAIVT | |
| | | VTYDSPPQRQQFLDVVKIPPTISHKLGFMSLHLL | |
| HPV E2- | 363 | MEAIAKRLDACAEQLLELAEENSTDLHKHVLHWKCMR | 161 |
| Variant | | HASVLLYKAKQMGLSHIGMQVVPPLKVSEAKGHNAIE | |
| H | | MQMHLESLLRTEYSMEPWTLQETSYAMWQTPPKRCFK | |
| | | KRGKTVEVKFDGCANNTMDYVVWTDVYVQDNDTWV | |
| | | KVHSMVDAKGIYYTCGQFKTYYVNFVKEAEKYGSTKH | |
| | | WEVCYGSTVICSPASVSSTTQEVSIPESTTYTPAQTSTLV | |
| | | SSSTKEDAVQTPPRKRARGVQQSPCNALCVAHIGPVDS | |
| | | GNHNLITNNHDQHQRRNNSNSSATPIVQFQGESNCLKC | |
| | | FRYRLNDRHRHLFDLISSTWHWASSKAPHKHAIVTVTY | |
| | | DSEEQRQQFLDVVKIPPTISHKLGFM | |
| HPV E2- | 359 | LDACAEQLLELAEENSTDLHKHVLHWKCMRHASVLLY | 162 |
| Variant | | KAKQMGLSHIGMQVVPPLKVSEAKGHNAIEMQMHLES | |
| I | | LLRTEYSMEPWTLQETSYAMWQTPPKRCFKKRGKTVE | |
| | | VKFDGCANNTMDYVVWTDVYVQDNDTWVKVHSMVD | |
| | | AKGIYYTCGQFKTYYVNFVKEAEKYGSTKHWEVCYGS | |
| | | TVICSPASVSSTTQEVSIPESTTYTPAQTSTLVSSSTKEDA | |
| | | VQTPPRKRARGVQQSPCNALCVAHIGPVDSGNHNLITN | |
| | | NHDQHQRRNNSNSSATPIVQFQGESNCLKCFRYRLNDR | |
| | | HRHLFDLISSTWHWASSKAPHKHAIVTVTYDSEEQRQQ | |
| | | FLDVVKIPPTISHKLGFMSLHLL | |
| HPVE2- | 360 | MEAIAKRLDACAEQLLELAEENSTDLHKHVLHWKCMR | 163 |
| Variant | | HASVLLYKAKQMGLSHIGMQVVPPLKVSEAKGHNAIE | |
| J | | MQMHLESLLRTEYSMEPWTLQETSYAMWQTPPKRCFK | |
| | | KRGKTVEVKFDGCANNTMDYVVWTDVYVQDNDTWV | |
| | | KVHSMVDGQFKTYYVNFVKEAEKYGSTKHWEVCYGS | |
| | | TVICSPASVSSTTQEVSIPESTTYTPAQTSTLVSSSTKEDA | |
| | | VQTPPRKRARGVQQSPCNALCVAHIGPVDSGNHNLITN | |
| | | NHDQHQRRNNSNSSATPIVQFQGESNCLKCFRYRLNDR | |
| | | HRHLFDLISSTWHWASSKAPHKHAIVTVTYDSEEQRQQ | |
| | | FLDVVKIPPTISHKLGFMSLHLL | |

HPV11 E7 Variant Sequences (Variants of SEQ ID NO: 106)

| | Size | Amino acid | |
|---|---|---|---|
| Antigen region | (aa) | sequence | SEQ ID NO: |
| HPV11 E7-Variant A | 15 | HCYEQLEDGGEDEVD | 164 |
| HPV11 E7-Variant B | 15 | HCYEQLEDSSESEVS | 165 |
| HPV11 E7-Variant C | 15 | HCYPQLPDSSEDEVD | 166 |
| HPV11 E7-Variant D | 15 | HCYPQLPDSSPDEVD | 167 |
| HPV11 E7-Variant E | 15 | HCYEQMEDGGEDEVD | 168 |
| HPV11 E7-Variant F | 10 | HCYEQLEDSS | 169 |
| HPV11 E7-Variant G | 12 | EQLEDSSEDEVD | 170 |
| HPV11 E7-Variant H | 13 | HCYLEDSSEDEVD | 171 |

HPV E4 Variant Sequences (Variants of SEQ ID NO: 107)

| Antigen | Size | | SEQ |
|---|---|---|---|
| region | (aa) | Amino acid sequence | ID NO: |
| HPV E4- | 56 | MADDGALHKKYPFLNLLHTPPHRPPPLCPQAPRKTQCKR | 172 |
| Variant A | | RLGNEHEEGNGPLATPC | |
| HPV E4- | 56 | MGDDSGLHKKYPFLNLLHTPPHRPPPLCPQGPRKTQCKR | 173 |
| Variant B | | RLGNEHEESNSPLGTPC | |
| HPV E4- | 56 | MADDSAMHKKYPFLNLLHTPPHRPPPLCPQAPRKTQCKR | 174 |
| Variant C | | RMGNEHEESNSPMATPC | |
| HPV E4- | 56 | MTDDSTLHKKYPFLNLLHTPPHRPPPLCPQAPRKAQCKR | 175 |
| Variant D | | RLGNEHEESNSPLAAPC | |
| HPV E4- | 56 | MADDSALHKKYPFLNLLHTPPHRPPPLCPQAPRKTQCKE | 176 |
| Variant E | | ELGNEHEESNSPLATPC | |
| HPV E4- | 53 | MADDSALHKKYPFLNLLHTPPHRPPPLCPQAPRKTQCKR | 177 |
| Variant F | | RLGNEHEESNSPLA | |
| HPV E4- | 50 | DSALHKKYPFLNLLHTPPHRPPPLCPQAPRKTQCKRRLGN | 178 |
| Variant G | | EHEESNSPLA | |
| HPV E4- | 54 | DDSALHKKYPFLNLLHTPPHRPPPLCPQAPRKTQCKRRLG | 179 |
| Variant H | | NEHEESNSPLATPC | |

HPV11 E6 Variant Sequences (Variants of SEQ ID NO: 108)

| | Size | | |
|---|---|---|---|
| Antigen region | (aa) | Amino acid sequence | SEQ ID NO: |
| HPV11 E6-Variant A | 15 | TAEIYGYGYKNLKVV | 180 |
| HPV11 E6-Variant B | 15 | TAEIYAMAMKNLKVV | 181 |
| HPV11 E6-Variant C | 15 | TAEIYAYAYDNLDVV | 182 |
| HPV11 E6-Variant D | 15 | TAEIYSYSYKNLKVV | 183 |
| HPV11 E6-Variant E | 15 | TAEIYAYAYKNLKAA | 184 |
| HPV11 E6-Variant F | 15 | TAEIYTYTYKNMKVV | 185 |
| HPV11 E6-Variant G | 14 | AEIYAYAYKNLKVV | 186 |
| HPV11 E6-Variant H | 12 | IYAYAYKNLKVV | 187 |
| HPV11 E6-Variant I | 10 | TAEIYAYAYK | 188 |

HPV6 E7 Variant Sequences (Variants of SEQ ID NO: 109)

| | Size | | SEQ ID |
|---|---|---|---|
| Antigen region | (aa) | Amino acid sequence | NO: |
| HPV6 E7-Variant A | 29 | QCTETDIREVDDLLLGTLNIVCPICAPKT | 189 |
| HPV6 E7-Variant B | 29 | QCTETDIREVQQLMMGTLNIVCPICAPKT | 190 |
| HPV6 E7-Variant C | 29 | QCNENDIREVQQLLLGTLNIVCPICAPKT | 191 |
| HPV6 E7-Variant D | 29 | QCTETDVREVQQLLLGTLNIVCPVCAPKT | 192 |
| HPV6 E7-Variant E | 29 | QCAEADIREVSSLLLGTLNIVCPICAPKT | 193 |
| HPV6 E7-Variant F | 28 | QCTETDIREVQQLLLGTNIVCPICAPKT | 194 |
| HPV6 E7-Variant G | 26 | QCTETDIREVQQLLLGTLNIVCPICA | 195 |
| HPV6 E7-Variant H | 25 | TETDIREVQQLLLGTLNIVCPICAP | 196 |

HPV6 E6 Variant Sequences (Variants of SEQ ID
NO: 110)

| | | | SEQ |
|---|---|---|---|
| | Size | | ID |
| Antigen region | (aa) | Amino acid sequence | NO: |
| HPV6 E6-Variant A | 15 | TAEILSLAYKQLKVL | 197 |
| HPV6 E6-Variant B | 15 | TAEIYSYAYDQLDVL | 198 |
| HPV6 E6-Variant C | 15 | TSEIYSYSYKQLKVL | 199 |
| HPV6 E6-Variant D | 15 | TAEIFSFAYEQLEVL | 200 |
| HPV6 E6-Variant E | 15 | TAEIISIAIKQVKVV | 201 |
| HPV6 E6-Variant F | 14 | TAEIYSYAYQLKVL | 202 |
| HPV6 E6-Variant G | 12 | TAEIYSYAYKQLK | 203 |
| HPV6 E6-Variant H | 11 | EIYSYAYKQLK | 204 |

HPV E4 Variant Sequences (Variants of SEQ ID NO: 111)

| | | | SEQ |
|---|---|---|---|
| Antigen | Size | | ID |
| region | (aa) | Amino acid sequence | NO: |
| HPV E4- | 55 | QCKRRLGNEHPPSNSPLATPCVWPTLDPWTVETTTSSLTI | 205 |
| Variant A | | TTSTKDGTTVTVQLR | |
| HPV E4- | 55 | QCKRRLGNEHEESNSPLATPCVWPTLDPWTVETTTSSLTI | 206 |
| Variant B | | NNSTKDGTTVTVQLR | |
| HPV E4- | 55 | QCDRRLGNEHEESNSPLATPCVWPNLDPWNVETTTSSLTI | 207 |
| Variant C | | TTSTKDGTTVTVQLR | |
| HPV E4- | 55 | QCKRRLGNEHEESNSPLATPCVWPTLDPWTVETTTSSLTI | 208 |
| Variant D | | TTSTKDGTTATAQLR | |
| HPV E4- | 55 | QCKEELGNEHEESNSPLATPCVWPTLDPWTVETTTGGLTI | 209 |
| Variant E | | TTSTKDGTTVTVQLR | |
| HPV E4- | 54 | QCKRRLGNEHEESNSPLATPCVWPTLDPWTVETTTSSLTI | 210 |
| Variant F | | TTSTKDGTTVTVQL | |
| HPV E4- | 52 | QCKRRLGNEHEESNSPLATPCVWPTLDPWTVETTTSSLTI | 211 |
| Variant G | | TTSTKDGTTVTV | |
| HPV E4- | 50 | KRRLGNEHEESNPLATPCVWPTLDPWTVETTTSSLTITTS | 212 |
| Variant H | | TKDGTTVTVQ | |

HPV11 E6 Variant Sequences (Variants of SEQ ID NO: 112)

| | Size | | SEQ ID |
|---|---|---|---|
| Antigen region | (aa) | Amino acid sequence | NO: |
| HPV11 E6-Variant A | 15 | TAEILAYAYKNLKVV | 213 |
| HPV11 E6-Variant B | 15 | TAEILALAYKNLKVV | 214 |
| HPV11 E6-Variant C | 15 | TAEIYGYGYKNLKVV | 215 |
| HPV11 E6-Variant D | 15 | TAEIYAYAYDNLDVV | 216 |
| HPV11 E6-Variant E | 15 | TVEIYAYAYKNLKVV | 217 |
| HPV11 E6-Variant F | 14 | AEIYAYAYKNLKVV | 218 |
| HPV11 E6-Variant G | 13 | EIYAYAYKNLKVV | 219 |
| HPV11 E6-Variant H | 12 | AEIYAYAYKLKV | 220 |

HPV E7 Variant Sequences (Variants of SEQ ID NO: 113)

| | | | SEQ |
|---|---|---|---|
| Antigen | Size | | ID |
| region | (aa) | Amino acid sequence | NO: |
| HPV E7- | 58 | MHGRHVTLKDIVLDLQEEDPVGLHCYEQLVDSSEDE | 221 |
| Variant A | | VDEVDGQDSQPLKQHFQIVTCC | |
| HPV E7- | 58 | MHGRHVTLKDIVLDLQPPDPVGLHCYEQLVDSSEDEV | 222 |
| Variant B | | SEVSGQDSQPLKQHFQIVTCC | |
| HPV E7- | 58 | MHGRHVNLKDIVLDLQPPDPVGLDCYEQLVDSSEDE | 223 |
| Variant C | | VDEVDGQDSQPLKQHFQIVTCC | |
| HPV E7- | 58 | MHGEHVTLKDIVLDLQPPDPAGLHCYEQMVDSSEDE | 224 |
| Variant D | | VDEVDGQDSQPLKQHFQIVTCC | |
| HPV E7- | 58 | MHGRHVTLKDIVLDLQPPDPVGLHCYEQMVDSSEDE | 225 |
| Variant E | | VDEVDGQDSQPLKKHFKIVTCC | |
| HPV E7- | 57 | MHGRHVTLKDIVLDLQPPDPVGLHCEQLVDSSEDEVD | 226 |
| Variant F | | EVDGQDSQPLKQHFQIVTCC | |
| HPV E7- | 55 | MHGRHVTLKDIVLDLQPPDPVGLHCYEQLVDSSEDEV | 227 |
| Variant G | | DEVDGQDSQPLKQHFQIV | |
| HPV E7- | 54 | GRHVTLKDIVLDLQPPDPVGLHCYEQVDSSEDEVDEV | 228 |
| Variant H | | DQDSQPLKQHFQIVTCC | |

HPV11 E7 Variant Sequences (Variants of SEQ ID NO: 114)

| | Size | | SEQ ID |
|---|---|---|---|
| Antigen region | (aa) | Amino acid sequence | NO: |
| HPV11 E7-Variant A | 15 | HCIEQLEDSSEDEVD | 229 |
| HPV11 E7-Variant B | 15 | HCYEQLEDGGEDEVD | 230 |
| HPV11 E7-Variant C | 15 | HCYEQLPDSSPDEVD | 231 |
| HPV11 E7-Variant D | 15 | HCYSQLEDSSEDEVD | 232 |
| HPV11 E7-Variant E | 15 | HCYEQLSDSSEDSVD | 233 |
| HPV11 E7-Variant F | 14 | HCYEQLEDSSEDEV | 234 |
| HPV11 E7-Variant G | 13 | YEQLEDSSEDEVD | 235 |
| HPV11 E7-Variant H | 12 | EQLEDSSEDEVD | 236 |

HPV E6 Variant Sequences (Variants of SEQ ID NO: 115)

| | | | SEQ |
|---|---|---|---|
| Antigen | Size | | ID |
| region | (aa) | Amino acid sequence | NO: |
| HPV E6- | 150 | MESANASTSSTTIDQLCKTFNLSMHTLQINCVFCKNALT | 237 |
| Variant A | | TAEIYSYAYKHLKVLFRGGYPYAACACCLEFHGKINQY | |
| | | AHFDYAGYTTTVEEETKQDILDVLIRCYLCHKPQCEVEK | |
| | | VKHILTKARFIKLNCTRKGRCLHCWTTCMEDMLP | |
| HPV E6- | 150 | MESANASTSATTIDQLCKTFNLSMHTLDINCVFCKNALT | 238 |
| Variant B | | TAEIYSYAYKHLKVLFRGGYPYAACACCLEFHGKINDY | |
| | | AHFDYAGYATTVEEETKDDILDVLIRCYLCHKPQCEVEK | |
| | | VKHILTKARFIKLNCTRKGRCLHCWTTCMEDMLP | |
| HPV E6- | 150 | MESANSSTSATTIDQLCKTFNLSMHTLQINCVFCKNALT | 239 |
| Variant C | | TAEIYSYAYKHLKVLFRGGYPYAACSCCLEFHGKINQYA | |
| | | HFDYAGYSTTVEEETKQDILDVLIRCYLCHKPQCEVEKV | |
| | | KHILTKARFIKLNCTRKGRCLHCWTTCMEDMLP | |
| HPV E6- | 150 | MESANASTSATTIDQLCKTFNLSMHTLQINCVFCKNALT | 240 |
| Variant D | | TGEIYSYAYKHLKVLFRGGHPHAACACCLEFHGKINQY | |
| | | AHFDYAGYATTVEEETKQDILDVLIRCHLCHKPQCEVEK | |
| | | VKHMLTKARFIKLNCTRKGRCLHCWTTCMEDMLP | |
| HPV E6- | 150 | MESANASTSATTIDQLCKTFNLSMHTLQINCVFCKNALT | 241 |
| Variant E | | TAEIYSYAYKHLKVLFRGGYPYVACVCCLEFHGKINQY | |
| | | AHFDYVGYVTTVEEETKQDILDVLIRCYLCHKPQCEVEK | |
| | | VKHILTKARFIKLNCTRKGRCLHCWTTCMEDMLP | |
| HPV E6- | 150 | MEGANASTSATTIDHLCKTFNLSMHTLQINCTFCKNALT | 242 |
| Variant F | | TAEIYSYGYKHLKVLFRGGYPYAACACCLEFHGKINQY | |
| | | AHFDYAGYATTVEEETKQDILDVLIECYLCHKPQCEVEK | |
| | | VKHILSKARFIKLNCTRKGRCLHCWTTCMEDMLP | |
| HPV E6- | 146 | SANASTSATTIDQLCKTFNLSMHTLQINCVFCKNALTTA | 243 |
| Variant G | | EIYSYAYKHLKVLFRGGYPYAACACCFHGKINQYAHFD | |
| | | YAGYATTVEEETKQDILDVLIRCYLCHKPQCEVEKVKHI | |
| | | LTKARFIKLNCTRKGRCLHCWTTCMEDMLP | |
| HPV E6- | 149 | MESANASTSATTIDQLCKTFNLSMHTLQINCVFCKNALT | 244 |
| Variant H | | TAEIYSYAYKHLKVLFRGGYPYAACACCLEFHGKINQY | |
| | | AHFDYAGYATTVEEETKQDILDVLIRCYLCHKPQCEVEK | |
| | | VKHILTKARFIKLNCTRGRCLHCWTTCMEDMLP | |
| HPV E6- | 148 | MESANASTSATTIDQLCKTFNLSMHTLQINCVFCKNALT | 245 |
| Variant I | | TAEIYSYAYKHLKVLFRGGYPYAACACCLEFHGKINQY | |
| | | AHFDYAGYATTVEEETKQDILDVLIRCYLCHKPQCEVEK | |
| | | VKHILTKFIKLNCTRKGRCLHCWTTCMEDMLP | |
| HPV E6- | 145 | MESANASTSATTIDQLCKTFNLSMHTLQINCVFCKNALT | 246 |
| Variant J | | TAEIYSYAYKHLKFRGGYPYAACACCLEFHGKINQYAH | |
| | | FDYAGYATTVEEETKQDILDVLIRCYLCHKPQCEVEKVK | |
| | | HILTKARFIKLNCTRKGRCLHCWTTCMED | |

1. A polynucleotide encoding a non-naturally occurring polypeptide comprising at least one epitope derived from a human papilloma virus 6 (HPV6) protein and at least one epitope derived from a human papilloma virus 11 (HPV11) protein.

2-59. (canceled)

60. The polynucleotide of claim 1, wherein:
the epitope derived from an HPV6 protein is:
an amino acid sequence having at least 80% identity with SEQ ID NO: 1;
an amino acid sequence having at least 80% identity with SEQ ID NO: 3 or 7;
an amino acid sequence having at least 80% identity with SEQ ID NO: 11; or
an amino acid sequence having at least 80% identity with SEQ ID NO: 5 or 9; and
the epitope derived from an HPV11 protein is:
an amino acid sequence having at least 80% identity with SEQ ID NO: 42; or
an amino acid sequence having at least 80% identity with SEQ ID NO: 45.

61. The polynucleotide of claim 60, wherein:
the epitope derived from an HPV6 protein is:
an amino acid sequence having at least 90% identity with SEQ ID NO: 1;
an amino acid sequence having at least 90% identity with SEQ ID NO: 3;
an amino acid sequence having at least 90% identity with SEQ ID NO: 11; or
an amino acid sequence having at least 90% identity with SEQ ID NO: 5; and
the epitope derived from an HPV11 protein is:
an amino acid sequence having at least 90% identity with SEQ ID NO: 42; or
an amino acid sequence having at least 90% identity with SEQ ID NO: 45.

62. The polynucleotide of claim 60, wherein:
the epitope derived from an HPV6 protein is:
an amino acid sequence having at least 90% identity with SEQ ID NO: 1;
an amino acid sequence having at least 90% identity with SEQ ID NO: 3;
an amino acid sequence having at least 90% identity with SEQ ID NO: 11; or
an amino acid sequence having at least 90% identity with SEQ ID NO: 9; and
the epitope derived from an HPV11 protein is:
an amino acid sequence having at least 90% identity with SEQ ID NO: 42; or
an amino acid sequence having at least 90% identity with SEQ ID NO: 45.

63. The polynucleotide of claim 60, wherein:
the epitope derived from an HPV6 protein is:
an amino acid sequence having at least 90% identity with SEQ ID NO: 1;
an amino acid sequence having at least 90% identity with SEQ ID NO: 7;
an amino acid sequence having at least 90% identity with SEQ ID NO: 11; or
an amino acid sequence having at least 90% identity with SEQ ID NO: 5; and
the epitope derived from an HPV11 protein is:
an amino acid sequence having at least 90% identity with SEQ ID NO: 42; or
an amino acid sequence having at least 90% identity with SEQ ID NO: 45.

64. The polynucleotide of claim 60, wherein:
the epitope derived from an HPV6 protein is:
an amino acid sequence having at least 90% identity with SEQ ID NO: 1;
an amino acid sequence having at least 90% identity with SEQ ID NO: 7;
an amino acid sequence having at least 90% identity with SEQ ID NO: 11; or
an amino acid sequence having at least 90% identity with SEQ ID NO: 9; and
the epitope derived from an HPV11 protein is:
an amino acid sequence having at least 90% identity with SEQ ID NO: 42; or
an amino acid sequence having at least 90% identity with SEQ ID NO: 45.

65. The polynucleotide of claim 60, wherein:
the epitope derived from an HPV6 protein is:
the amino acid sequence of SEQ ID NO: 1;
the amino acid sequence of SEQ ID NO: 3;
the amino acid sequence of SEQ ID NO: 11; or
the amino acid sequence of SEQ ID NO: 5; and
the epitope derived from an HPV11 protein is:
the amino acid sequence of SEQ ID NO: 42; or
the amino acid sequence of SEQ ID NO: 45.

66. The polynucleotide of claim 60, wherein:
the epitope derived from an HPV6 protein is:
the amino acid sequence of SEQ ID NO: 1;
the amino acid sequence of SEQ ID NO: 3;
the amino acid sequence of SEQ ID NO: 11; or
the amino acid sequence of SEQ ID NO: 9; and
the epitope derived from an HPV11 protein is:
the amino acid sequence of SEQ ID NO: 42; or
the amino acid sequence of SEQ ID NO: 45.

67. The polynucleotide of claim 60, wherein:
the epitope derived from an HPV6 protein is:
the amino acid sequence of SEQ ID NO: 1;
the amino acid sequence of SEQ ID NO: 7;
the amino acid sequence of SEQ ID NO: 11; or
the amino acid sequence of SEQ ID NO: 5; and
the epitope derived from an HPV11 protein is:
the amino acid sequence of SEQ ID NO: 42; or
the amino acid sequence of SEQ ID NO: 45.

68. The polynucleotide of claim 60, wherein:
the epitope derived from an HPV6 protein is:
the amino acid sequence of SEQ ID NO: 1;
the amino acid sequence of SEQ ID NO: 7;
the amino acid sequence of SEQ ID NO: 11; or
the amino acid sequence of SEQ ID NO: 9; and
the epitope derived from an HPV11 protein is:
the amino acid sequence of SEQ ID NO: 42; or
the amino acid sequence of SEQ ID NO: 45.

69. A polynucleotide encoding a non-naturally occurring polypeptide comprising HPV epitopes derived from any three of:
an HPV6 E2 protein;
an HPV6 E4 protein;
an HPV6 E6 protein; and
an HPV6 E7 protein.

70. The polynucleotide of claim 69, wherein:
the HPV epitope derived from an HPV6 E2 protein, if present, comprises an amino acid sequence having at least 80% identity with SEQ ID NO: 1;
the HPV epitope derived from an HPV6 E4 protein, if present, comprises an amino acid sequence having at least 80% identity with SEQ ID NO: 3 or 7;
the HPV epitope derived from an HPV6 E6 protein, if present, comprises an amino acid sequence having at least 80% identity with SEQ ID NO: 11; and
the HPV epitope derived from an HPV6 E7 protein, if present, comprises an amino acid sequence having at least 80% identity with SEQ ID NO: 5 or 9.

71. The polynucleotide of claim 70, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 3;
the HPV epitope derived from an HPV6 E6 protein, if present, comprises an amino acid sequence having at least 90% identity with SEQ ID NO: 11; and
the HPV epitope derived from an HPV6 E7 protein, if present, comprises an amino acid sequence having at least 90% identity with SEQ ID NO: 5.

72. The polynucleotide of claim 70, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 3;
the HPV epitope derived from an HPV6 E6 protein, if present, comprises an amino acid sequence having at least 90% identity with SEQ ID NO: 11; and
the HPV epitope derived from an HPV6 E7 protein, if present, comprises an amino acid sequence having at least 90% identity with SEQ ID NO: 9.

73. The polynucleotide of claim 70, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 7;
the HPV epitope derived from an HPV6 E6 protein, if present, comprises an amino acid sequence having at least 90% identity with SEQ ID NO: 11; and
the HPV epitope derived from an HPV6 E7 protein, if present, comprises an amino acid sequence having at least 90% identity with SEQ ID NO: 5.

74. The polynucleotide of claim 70, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 7;
the HPV epitope derived from an HPV6 E6 protein, if present, comprises an amino acid sequence having at least 90% identity with SEQ ID NO: 11; and
the HPV epitope derived from an HPV6 E7 protein, if present, comprises an amino acid sequence having at least 90% identity with SEQ ID NO: 9.

75. The polynucleotide of claim 70, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 3;
the HPV epitope derived from an HPV6 E6 protein, if present, comprises the amino acid sequence of SEQ ID NO: 11; and
the HPV epitope derived from an HPV6 E7 protein, if present, comprises the amino acid sequence of SEQ ID NO: 5.

76. The polynucleotide of claim 70, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 3;
the HPV epitope derived from an HPV6 E6 protein, if present, comprises the amino acid sequence of SEQ ID NO: 11; and
the HPV epitope derived from an HPV6 E7 protein, if present, comprises the amino acid sequence of SEQ ID NO: 9.

77. The polynucleotide of claim 70, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 7;
the HPV epitope derived from an HPV6 E6 protein, if present, comprises the amino acid sequence of SEQ ID NO: 11; and
the HPV epitope derived from an HPV6 E7 protein, if present, comprises the amino acid sequence of SEQ ID NO: 5.

78. The polynucleotide of claim 70, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 7;
the HPV epitope derived from an HPV6 E6 protein, if present, comprises the amino acid sequence of SEQ ID NO: 11; and
the HPV epitope derived from an HPV6 E7 protein, if present, comprises the amino acid sequence of SEQ ID NO: 9.

79. A polynucleotide encoding a non-naturally occurring polypeptide comprising HPV epitopes derived from any four of:
an HPV6 E2 protein;
an HPV6 E4 protein;
an HPV6 E6 protein;
an HPV6 E7 protein;
an HPV11 E6; and
an HPV11 E7 protein.

80. The polynucleotide of claim 79, wherein:
the HPV epitope derived from an HPV6 E2 protein, if present, comprises an amino acid sequence having at least 80% identity with SEQ ID NO: 1;
the HPV epitope derived from an HPV6 E4 protein, if present, comprises an amino acid sequence having at least 80% identity with SEQ ID NO: 3 or 7;
the HPV epitope derived from an HPV6 E6 protein, if present, comprises an amino acid sequence having at least 80% identity with SEQ ID NO: 11; and
the HPV epitope derived from an HPV6 E7 protein, if present, comprises an amino acid sequence having at least 80% identity with SEQ ID NO: 5 or 9;
the HPV epitope derived from an HPV11 E6 protein, if present, comprises an amino acid sequence having at least 80% identity with SEQ ID NO: 42; and
the HPV epitope derived from an HPV11 E7 protein, if present, comprises an amino acid sequence having at least 80% identity with SEQ ID NO: 45.

81. The polynucleotide of claim 80, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 3;
the HPV epitope derived from an HPV6 E6 protein, if present, comprises an amino acid sequence having at least 90% identity with SEQ ID NO: 11;
the HPV epitope derived from an HPV6 E7 protein, if present, comprises an amino acid sequence having at least 90% identity with SEQ ID NO: 5;
the HPV epitope derived from an HPV11 E6 protein, if present, comprises an amino acid sequence having at least 90% identity with SEQ ID NO: 42; and
the HPV epitope derived from an HPV11 E7 protein, if present, comprises an amino acid sequence having at least 90% identity with SEQ ID NO: 45.

82. The polynucleotide of claim 80, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 3;
the HPV epitope derived from an HPV6 E6 protein, if present, comprises an amino acid sequence having at least 90% identity with SEQ ID NO: 11;
the HPV epitope derived from an HPV6 E7 protein, if present, comprises an amino acid sequence having at least 90% identity with SEQ ID NO: 9;
the HPV epitope derived from an HPV11 E6 protein, if present, comprises an amino acid sequence having at least 90% identity with SEQ ID NO: 42; and
the HPV epitope derived from an HPV11 E7 protein, if present, comprises an amino acid sequence having at least 90% identity with SEQ ID NO: 45.

83. The polynucleotide of claim 80, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 7;
the HPV epitope derived from an HPV6 E6 protein, if present, comprises an amino acid sequence having at least 90% identity with SEQ ID NO: 11;
the HPV epitope derived from an HPV6 E7 protein, if present, comprises an amino acid sequence having at least 90% identity with SEQ ID NO: 5;
the HPV epitope derived from an HPV11 E6 protein, if present, comprises an amino acid sequence having at least 90% identity with SEQ ID NO: 42; and
the HPV epitope derived from an HPV11 E7 protein, if present, comprises an amino acid sequence having at least 90% identity with SEQ ID NO: 45.

84. The polynucleotide of claim 80, wherein:
the HPV epitope derived from an HPV6 E2 protein, if present, comprises an amino acid sequence having at least 90% identity with SEQ ID NO: 1;
the HPV epitope derived from an HPV6 E4 protein, if present, comprises an amino acid sequence having at least 90% identity with SEQ ID NO: 7;
the HPV epitope derived from an HPV6 E6 protein, if present, comprises an amino acid sequence having at least 90% identity with SEQ ID NO: 11;
the HPV epitope derived from an HPV6 E7 protein, if present, comprises an amino acid sequence having at least 90% identity with SEQ ID NO: 9;
the HPV epitope derived from an HPV11 E6 protein, if present, comprises an amino acid sequence having at least 90% identity with SEQ ID NO: 42; and
the HPV epitope derived from an HPV11 E7 protein, if present, comprises an amino acid sequence having at least 90% identity with SEQ ID NO: 45.

85. The polynucleotide of claim 80, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 3;
the HPV epitope derived from an HPV6 E6 protein, if present, comprises the amino acid sequence of SEQ ID NO: 11;
the HPV epitope derived from an HPV6 E7 protein, if present, comprises the amino acid sequence of SEQ ID NO: 5;
the HPV epitope derived from an HPV11 E6 protein, if present, comprises the amino acid sequence of SEQ ID NO: 42; and
the HPV epitope derived from an HPV11 E7 protein, if present, comprises the amino acid sequence of SEQ ID NO: 45.

86. The polynucleotide of claim 80, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 3;
the HPV epitope derived from an HPV6 E6 protein, if present, comprises the amino acid sequence of SEQ ID NO: 11;
the HPV epitope derived from an HPV6 E7 protein, if present, comprises the amino acid sequence of SEQ ID NO: 9;
the HPV epitope derived from an HPV11 E6 protein, if present, comprises the amino acid sequence of SEQ ID NO: 42; and
the HPV epitope derived from an HPV11 E7 protein, if present, comprises the amino acid sequence of SEQ ID NO: 45.

87. The polynucleotide of claim 80, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 7;
the HPV epitope derived from an HPV6 E6 protein, if present, comprises the amino acid sequence of SEQ ID NO: 11;
the HPV epitope derived from an HPV6 E7 protein, if present, comprises the amino acid sequence of SEQ ID NO: 5;
the HPV epitope derived from an HPV11 E6 protein, if present, comprises the amino acid sequence of SEQ ID NO: 42; and
the HPV epitope derived from an HPV11 E7 protein, if present, comprises the amino acid sequence of SEQ ID NO: 45.

88. The polynucleotide of claim 80, wherein:
the HPV epitope derived from an HPV6 E2 protein, if present, comprises the amino acid sequence of SEQ ID NO: 1;
the HPV epitope derived from an HPV6 E4 protein, if present, comprises the amino acid sequence of SEQ ID NO: 7;
the HPV epitope derived from an HPV6 E6 protein, if present, comprises the amino acid sequence of SEQ ID NO: 11;
the HPV epitope derived from an HPV6 E7 protein, if present, comprises the amino acid sequence of SEQ ID NO: 9;
the HPV epitope derived from an HPV11 E6 protein, if present, comprises the amino acid sequence of SEQ ID NO: 42; and
the HPV epitope derived from an HPV11 E7 protein, if present, comprises the amino acid sequence of SEQ ID NO: 45.

89. A polynucleotide encoding a non-naturally occurring polypeptide comprising HPV epitopes derived from each of:
an HPV6 E2 protein;
an HPV6 E4 protein;
an HPV6 E6 protein; and
an HPV6 E7 protein.

90. The polynucleotide of claim 89, wherein:
the HPV epitope derived from an HPV6 E2 protein comprises an amino acid sequence having at least 80% identity with SEQ ID NO: 1;
the HPV epitope derived from an HPV6 E4 protein comprises an amino acid sequence having at least 80% identity with SEQ ID NO: 3 or 7;
the HPV epitope derived from an HPV6 E6 protein comprises an amino acid sequence having at least 80% identity with SEQ ID NO: 11; and
the HPV epitope derived from an HPV6 E7 protein comprises an amino acid sequence having at least 80% identity with SEQ ID NO: 5 or 9.

91. The polynucleotide of claim 90, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 3;
the HPV epitope derived from an HPV6 E6 protein comprises an amino acid sequence having at least 90% identity with SEQ ID NO: 11; and
the HPV epitope derived from an HPV6 E7 protein comprises an amino acid sequence having at least 90% identity with SEQ ID NO: 5.

92. The polynucleotide of claim 90, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 3;
the HPV epitope derived from an HPV6 E6 protein comprises an amino acid sequence having at least 90% identity with SEQ ID NO: 11; and
the HPV epitope derived from an HPV6 E7 protein comprises an amino acid sequence having at least 90% identity with SEQ ID NO: 9.

93. The polynucleotide of claim 90, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 7;
the HPV epitope derived from an HPV6 E6 protein comprises an amino acid sequence having at least 90% identity with SEQ ID NO: 11; and
the HPV epitope derived from an HPV6 E7 protein comprises an amino acid sequence having at least 90% identity with SEQ ID NO: 5.

94. The polynucleotide of claim 90, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 7;
the HPV epitope derived from an HPV6 E6 protein comprises an amino acid sequence having at least 90% identity with SEQ ID NO: 11; and
the HPV epitope derived from an HPV6 E7 protein comprises an amino acid sequence having at least 90% identity with SEQ ID NO: 9.

95. The polynucleotide of claim 90, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 3;
the HPV epitope derived from an HPV6 E6 protein comprises the amino acid sequence of SEQ ID NO: 11; and
the HPV epitope derived from an HPV6 E7 protein comprises the amino acid sequence of SEQ ID NO: 5.

96. The polynucleotide of claim 90, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 3;
the HPV epitope derived from an HPV6 E6 protein comprises the amino acid sequence of SEQ ID NO: 11; and
the HPV epitope derived from an HPV6 E7 protein comprises the amino acid sequence of SEQ ID NO: 9.

97. The polynucleotide of claim 90, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 7;
the HPV epitope derived from an HPV6 E6 protein comprises the amino acid sequence of SEQ ID NO: 11; and
the HPV epitope derived from an HPV6 E7 protein comprises the amino acid sequence of SEQ ID NO: 5.

98. The polynucleotide of claim 90, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 7;
the HPV epitope derived from an HPV6 E6 protein comprises the amino acid sequence of SEQ ID NO: 11; and
the HPV epitope derived from an HPV6 E7 protein comprises the amino acid sequence of SEQ ID NO: 9.

99. The polynucleotide of claim 89, wherein the non-naturally occurring polypeptide further comprises epitopes derived from an HPV11 E6 protein and an HPV11 E7 protein.

100. The polynucleotide of claim 99, wherein:
the HPV epitope derived from an HPV6 E2 protein comprises an amino acid sequence having at least 80% identity with SEQ ID NO: 1;
the HPV epitope derived from an HPV6 E4 protein comprises an amino acid sequence having at least 80% identity with SEQ ID NO: 3 or 7;
the HPV epitope derived from an HPV6 E6 protein comprises an amino acid sequence having at least 80% identity with SEQ ID NO: 11;
the HPV epitope derived from an HPV6 E7 protein comprises an amino acid sequence having at least 80% identity with SEQ ID NO: 5 or 9;
the HPV epitope derived from an HPV11 E6 protein comprises an amino acid sequence having at least 80% identity with SEQ ID NO: 42; and
the HPV epitope derived from an HPV11 E7 protein comprises an amino acid sequence having at least 80% identity with SEQ ID NO: 45.

101. The polynucleotide of claim 100, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 3;
the HPV epitope derived from an HPV6 E6 protein comprises an amino acid sequence having at least 90% identity with SEQ ID NO: 11;
the HPV epitope derived from an HPV6 E7 protein comprises an amino acid sequence having at least 90% identity with SEQ ID NO: 5;
the HPV epitope derived from an HPV11 E6 protein comprises an amino acid sequence having at least 90% identity with SEQ ID NO: 42; and
the HPV epitope derived from an HPV11 E7 protein comprises an amino acid sequence having at least 90% identity with SEQ ID NO: 45.

102. The polynucleotide of claim 100, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 3;
the HPV epitope derived from an HPV6 E6 protein comprises an amino acid sequence having at least 90% identity with SEQ ID NO: 11;
the HPV epitope derived from an HPV6 E7 protein comprises an amino acid sequence having at least 90% identity with SEQ ID NO: 9;
the HPV epitope derived from an HPV11 E6 protein comprises an amino acid sequence having at least 90% identity with SEQ ID NO: 42; and
the HPV epitope derived from an HPV11 E7 protein comprises an amino acid sequence having at least 90% identity with SEQ ID NO: 45.

103. The polynucleotide of claim 100, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 7;
the HPV epitope derived from an HPV6 E6 protein comprises an amino acid sequence having at least 90% identity with SEQ ID NO: 11;
the HPV epitope derived from an HPV6 E7 protein comprises an amino acid sequence having at least 90% identity with SEQ ID NO: 5;
the HPV epitope derived from an HPV11 E6 protein comprises an amino acid sequence having at least 90% identity with SEQ ID NO: 42; and
the HPV epitope derived from an HPV11 E7 protein comprises an amino acid sequence having at least 90% identity with SEQ ID NO: 45.

104. The polynucleotide of claim 100, wherein:
the HPV epitope derived from an HPV6 E2 protein comprises an amino acid sequence having at least 90% identity with SEQ ID NO: 1;
the HPV epitope derived from an HPV6 E4 protein comprises an amino acid sequence having at least 90% identity with SEQ ID NO: 7;
the HPV epitope derived from an HPV6 E6 protein comprises an amino acid sequence having at least 90% identity with SEQ ID NO: 11;
the HPV epitope derived from an HPV6 E7 protein comprises an amino acid sequence having at least 90% identity with SEQ ID NO: 9;
the HPV epitope derived from an HPV11 E6 protein comprises an amino acid sequence having at least 90% identity with SEQ ID NO: 42; and
the HPV epitope derived from an HPV11 E7 protein comprises an amino acid sequence having at least 90% identity with SEQ ID NO: 45.

105. The polynucleotide of claim 100, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 3;
the HPV epitope derived from an HPV6 E6 protein comprises the amino acid sequence of SEQ ID NO: 11;
the HPV epitope derived from an HPV6 E7 protein comprises the amino acid sequence of SEQ ID NO: 5;
the HPV epitope derived from an HPV11 E6 protein comprises the amino acid sequence of SEQ ID NO: 42; and
the HPV epitope derived from an HPV11 E7 protein comprises the amino acid sequence of SEQ ID NO: 45.

106. The polynucleotide of claim 100, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 3;
the HPV epitope derived from an HPV6 E6 protein comprises the amino acid sequence of SEQ ID NO: 11;
the HPV epitope derived from an HPV6 E7 protein comprises the amino acid sequence of SEQ ID NO: 9;
the HPV epitope derived from an HPV11 E6 protein comprises the amino acid sequence of SEQ ID NO: 42; and
the HPV epitope derived from an HPV11 E7 protein comprises the amino acid sequence of SEQ ID NO: 45.

107. The polynucleotide of claim 100, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 7;
the HPV epitope derived from an HPV6 E6 protein comprises the amino acid sequence of SEQ ID NO: 11;
the HPV epitope derived from an HPV6 E7 protein comprises the amino acid sequence of SEQ ID NO: 5;
the HPV epitope derived from an HPV11 E6 protein comprises the amino acid sequence of SEQ ID NO: 42; and
the HPV epitope derived from an HPV11 E7 protein comprises the amino acid sequence of SEQ ID NO: 45.

108. The polynucleotide of claim 100, wherein:
the HPV epitope derived from an HPV6 E2 protein comprises the amino acid sequence of SEQ ID NO: 1;
the HPV epitope derived from an HPV6 E4 protein comprises the amino acid sequence of SEQ ID NO: 7;
the HPV epitope derived from an HPV6 E6 protein comprises the amino acid sequence of SEQ ID NO: 11;
the HPV epitope derived from an HPV6 E7 protein comprises the amino acid sequence of SEQ ID NO: 9;
the HPV epitope derived from an HPV11 E6 protein comprises the amino acid sequence of SEQ ID NO: 42; and
the HPV epitope derived from an HPV11 E7 protein comprises the amino acid sequence of SEQ ID NO: 45.

109. The polynucleotide of claim 99, wherein the non-naturally occurring polypeptide comprises, in order from N-terminus to C-terminus, epitopes derived from HPV6 E2, HPV11 E7, HPV6 E4, HPV11 E6, HPV6 E7, HPV6 E6, HPV6 E4, HPV11 E6, HPV6 E7, HPV11 E7, and HPV6 E6.

110. A polynucleotide encoding a non-naturally occurring polypeptide comprising:
an amino acid sequence having at least 80% identity with SEQ ID NO: 3; and an amino acid sequence having at least 80% identity with SEQ ID NO: 7;
an amino acid sequences having at least 80% identity with SEQ ID NO: 11; and an amino acid sequence having at least 80% identity with SEQ ID NO: 40; or
an amino acid sequences having at least 80% identity with SEQ ID NO: 5; and an amino acid sequence having at least 80% identity with SEQ ID NO: 9.

111. The polynucleotide of claim 110, wherein the non-naturally occurring polypeptide comprises: an amino acid sequence having at least 80% identity with SEQ ID NO: 3; and an amino acid sequence having at least 80% identity with SEQ ID NO: 7.

112. The polynucleotide of claim 111, wherein the non-naturally occurring polypeptide comprises: an amino acid sequence having at least 90% identity with SEQ ID NO: 3; and an amino acid sequence having at least 90% identity with SEQ ID NO: 7.

113. The polynucleotide of claim 112, wherein the non-naturally occurring polypeptide further comprises: an amino acid sequence having at least 90% identity with SEQ ID NO: 11; and an amino acid sequence having at least 90% identity with SEQ ID NO: 40.

114. The polynucleotide of claim 112, wherein the non-naturally occurring polypeptide further comprises: an amino acid sequence having at least 90% identity with SEQ ID NO: 5; and an amino acid sequence having at least 90% identity with SEQ ID NO: 9.

115. The polynucleotide of claim 111, wherein the non-naturally occurring polypeptide comprises the amino acid sequences of SEQ ID NO: 3 and SEQ ID NO: 7.

116. The polynucleotide of claim 115, wherein the non-naturally occurring polypeptide further comprises the amino acid sequences of SEQ ID NO: 11 and SEQ ID NO: 40.

117. The polynucleotide of claim 115, wherein the non-naturally occurring polypeptide further comprises the amino acid sequences of SEQ ID NO: 5 and SEQ ID NO: 9.

118. The polynucleotide of claim 110, wherein the non-naturally occurring polypeptide comprises: an amino acid sequence having at least 80% identity with SEQ ID NO: 11; and an amino acid sequence having at least 80% identity with SEQ ID NO: 40.

119. The polynucleotide of claim 118, wherein the non-naturally occurring polypeptide comprises: an amino acid sequence having at least 90% identity with SEQ ID NO: 11; and an amino acid sequence having at least 90% identity with SEQ ID NO: 40.

120. The polynucleotide of claim 119, wherein the non-naturally occurring polypeptide further comprises: an amino acid sequence having at least 90% identity with SEQ ID NO: 3; and an amino acid sequence having at least 90% identity with SEQ ID NO: 7.

121. The polynucleotide of claim 119, wherein the non-naturally occurring polypeptide further comprises: an amino acid sequence having at least 90% identity with SEQ ID NO: 5; and an amino acid sequence having at least 90% identity with SEQ ID NO: 9.

122. The polynucleotide of claim 118, wherein the non-naturally occurring polypeptide comprises the amino acid sequences of SEQ ID NO: 11 and SEQ ID NO: 40.

123. The polynucleotide of claim 122, wherein the non-naturally occurring polypeptide further comprises the amino acid sequences of SEQ ID NO: 3 and SEQ ID NO: 7.

124. The polynucleotide of claim 122, wherein the non-naturally occurring polypeptide further comprises the amino acid sequences of SEQ ID NO: 5 and SEQ ID NO: 9.

125. The polynucleotide of claim 110, wherein the non-naturally occurring polypeptide comprises: an amino acid sequence having at least 80% identity with SEQ ID NO: 5; and an amino acid sequence having at least 80% identity with SEQ ID NO: 9.

126. The polynucleotide of claim 125, wherein the non-naturally occurring polypeptide comprises: an amino acid sequence having at least 90% identity with SEQ ID NO: 5; and an amino acid sequence having at least 90% identity with SEQ ID NO: 9.

127. The polynucleotide of claim 126, wherein the non-naturally occurring polypeptide further comprises: an amino acid sequence having at least 90% identity with SEQ ID NO: 3 and an amino acid sequence having at least 90% identity with SEQ ID NO: 7.

128. The polynucleotide of claim 126, wherein the non-naturally occurring polypeptide further comprises: an amino acid sequence having at least 90% identity with SEQ ID NO: 11; and an amino acid sequence having at least 90% identity with SEQ ID NO: 40.

129. The polynucleotide of claim 125, wherein the non-naturally occurring polypeptide comprises the amino acid sequences of SEQ ID NO: 5 and SEQ ID NO: 9.

130. The polynucleotide of claim 129, wherein the non-naturally occurring polypeptide further comprises the amino acid sequences of SEQ ID NO: 3 and SEQ ID NO: 7.

131. The polynucleotide of claim 129, wherein the non-naturally occurring polypeptide further comprises the amino acid sequences of SEQ ID NO: 11 and SEQ ID NO: 40.

132. A polynucleotide encoding a non-naturally occurring polypeptide comprising the amino acid sequences of each of:
SEQ ID NO: 105, or a functional variant thereof;
SEQ ID NO: 106, or a functional variant thereof;
SEQ ID NO: 107, or a functional variant thereof;
SEQ ID NO: 108, or a functional variant thereof;
SEQ ID NO: 109, or a functional variant thereof;
SEQ ID NO: 110, or a functional variant thereof;
SEQ ID NO: 111, or a functional variant thereof;
SEQ ID NO: 112, or a functional variant thereof;
SEQ ID NO: 113, or a functional variant thereof;
SEQ ID NO: 114, or a functional variant thereof; and
SEQ ID NO: 115, or a functional variant thereof.

133. The polynucleotide of claim 132, wherein the non-naturally occurring polypeptide comprises:
an amino acid sequence having at least 80% identity with SEQ ID NO: 105;
an amino acid sequence having at least 80% identity with SEQ ID NO: 106;
an amino acid sequence having at least 80% identity with SEQ ID NO: 107;
an amino acid sequence having at least 80% identity with SEQ ID NO: 108;
an amino acid sequence having at least 80% identity with SEQ ID NO: 109;
an amino acid sequence having at least 80% identity with SEQ ID NO: 110;
an amino acid sequence having at least 80% identity with SEQ ID NO: 111;
an amino acid sequence having at least 80% identity with SEQ ID NO: 112;
an amino acid sequence having at least 80% identity with SEQ ID NO: 113;
an amino acid sequence having at least 80% identity with SEQ ID NO: 114; and
an amino acid sequence having at least 80% identity with SEQ ID NO: 115.

133. The polynucleotide of claim 132, wherein the non-naturally occurring polypeptide comprises:
an amino acid sequence having at least 90% identity with SEQ ID NO: 105;
an amino acid sequence having at least 90% identity with SEQ ID NO: 106;
an amino acid sequence having at least 90% identity with SEQ ID NO: 107;
an amino acid sequence having at least 90% identity with SEQ ID NO: 108;
an amino acid sequence having at least 90% identity with SEQ ID NO: 109;
an amino acid sequence having at least 90% identity with SEQ ID NO: 110;
an amino acid sequence having at least 90% identity with SEQ ID NO: 111;
an amino acid sequence having at least 90% identity with SEQ ID NO: 112;
an amino acid sequence having at least 90% identity with SEQ ID NO: 113;
an amino acid sequence having at least 90% identity with SEQ ID NO: 114; and
an amino acid sequence having at least 90% identity with SEQ ID NO: 115.

135. The polynucleotide of claim 132, wherein the non-naturally occurring polypeptide comprises:
an amino acid sequence having at least 95% identity with SEQ ID NO: 105;
an amino acid sequence having at least 95% identity with SEQ ID NO: 106;
an amino acid sequence having at least 95% identity with SEQ ID NO: 107;
an amino acid sequence having at least 95% identity with SEQ ID NO: 108;
an amino acid sequence having at least 95% identity with SEQ ID NO: 109;
an amino acid sequence having at least 95% identity with SEQ ID NO: 110;
an amino acid sequence having at least 95% identity with SEQ ID NO: 111;
an amino acid sequence having at least 95% identity with SEQ ID NO: 112;
an amino acid sequence having at least 95% identity with SEQ ID NO: 113;
an amino acid sequence having at least 95% identity with SEQ ID NO: 114 and
an amino acid sequence having at least 95% identity with SEQ ID NO: 115.

136. The polynucleotide of claim 132, wherein the non-naturally occurring polypeptide comprises the amino acid sequences of each of:
SEQ ID NO: 105, or a conservatively substituted variant thereof;
SEQ ID NO: 106, or a conservatively substituted variant thereof;
SEQ ID NO: 107, or a conservatively substituted variant thereof;
SEQ ID NO: 108, or a conservatively substituted variant thereof;
SEQ ID NO: 109, or a conservatively substituted variant thereof;
SEQ ID NO: 110, or a conservatively substituted variant thereof;
SEQ ID NO: 111, or a conservatively substituted variant thereof;
SEQ ID NO: 112, or a conservatively substituted variant thereof;
SEQ ID NO: 113, or a conservatively substituted variant thereof;
SEQ ID NO: 114, or a conservatively substituted variant thereof; and
SEQ ID NO: 115, or a conservatively substituted variant thereof.

137. The polynucleotide of claim 132, wherein the non-naturally occurring polypeptide comprises the amino acid sequences of each of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, and SEQ ID NO: 115.

138. The polynucleotide of claim 132, wherein the polynucleotide comprises the nucleic acid sequences of SEQ ID NO: 2, SEQ ID NO: 4, SEQ ID NO: 6, SEQ ID NO: 8, SEQ ID NO: 10, and SEQ ID NO: 12.

139. A polynucleotide encoding a non-naturally occurring polypeptide comprising:
SEQ ID NO: 66, or a functional variant thereof;
SEQ ID NO: 68, or a functional variant thereof;
SEQ ID NO: 70, or a functional variant thereof;
SEQ ID NO: 72, or a functional variant thereof; or
SEQ ID NO: 74, or a functional variant thereof.

140. The polynucleotide of claim 139, wherein the non-naturally occurring polypeptide comprises the amino acid sequence of SEQ ID NO: 68, or a functional variant thereof.

141. The polynucleotide of claim 140, wherein the non-naturally occurring polypeptide comprises an amino acid sequence having at least 80% identity with SEQ ID NO: 68.

142. The polynucleotide of claim 140, wherein the non-naturally occurring polypeptide comprises an amino acid sequence having at least 90% identity with SEQ ID NO: 68.

143. The polynucleotide of claim 140, wherein the non-naturally occurring polypeptide comprises an amino acid sequence having at least 95% identity with SEQ ID NO: 68.

144. The polynucleotide of claim 140, wherein the non-naturally occurring polypeptide comprises the amino acid sequence of SEQ ID NO: 68, or a conservatively substituted variant thereof.

145. The polynucleotide of claim 140, wherein the non-naturally occurring polypeptide comprises the amino acid sequence of SEQ ID NO: 68.

146. The polynucleotide of claim 140, wherein the polynucleotide comprises the nucleic acid sequence of SEQ ID NO: 69.

147. A non-naturally occurring polypeptide encoded by the polynucleotide of claim 1.

148. A vector comprising the polynucleotide of claim 1.

149. The vector of claim 148, wherein the vector is a plasmid, a viral, or a non-viral vector.

150. The vector of claim 149, wherein the vector is an adenoviral vector.

151. The vector of claim 150, wherein the adenoviral vector is a gorilla adenoviral vector.

152. A method of inducing an anti-HPV immune response in a subject in need thereof comprising administering a therapeutically effective amount of the vector of claim 148 to the subject.

153. A method of treating an HPV-associated disease or disorder in a subject in need thereof comprising administering a therapeutically effective amount of the vector of claim 148 to the subject.

154. The method of claim 153, wherein the HPV-associated disease or disorder is an HPV6-associated disease or disorder or an HPV11-associated disease or disorder.

155. The method of claim 154, wherein the HPV-associated disease or disorder is Recurrent Respiratory Papillomatosis (RRP), anogenital warts, lower genital tract neoplasia, cervical cancer, vulvar cancer, anal cancer, penile cancer, or head and neck cancer.

156. The method of claim 155, wherein the disease or disorder is Recurrent Respiratory Papillomatosis (RRP).


SEQUENCE LISTING (153 sequences)

SEQ ID NO: 1 - protein, 368 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLL

SEQ ID NO: 2 - DNA, 1104 bp, synthetic construct
  source 1..1104: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctc

SEQ ID NO: 3 - protein, 56 aa, synthetic construct
MADDSALHKKYPFLNLLHTPPHRPPPLCPQAPRKTQCKRRLGNEHEESNSPLATPC

SEQ ID NO: 4 - DNA, 168 bp, synthetic construct
  source 1..168: mol_type = other DNA
atggcagacgattctgcactgcataaaaagtacccattcctgaacctgctgcataccccaccgcatcgcccaccgccactgtgtccgcaagctccacgcaagacccaatgcaagcgccgtctgggtaacgagcacgaggaatccaactccccgctggctactccgtgt

SEQ ID NO: 5 - protein, 29 aa, synthetic construct
QCTETDIREVQQLLLGTLNIVCPICAPKT

SEQ ID NO: 6 - DNA, 87 bp, synthetic construct
  source 1..87: mol_type = other DNA
caatgcacagagacagacattagagaagtgcaacagctcctgctcggcacactgaatatcgtctgccctatctgtgcccctaagaca

SEQ ID NO: 7 - protein, 55 aa, synthetic construct
QCKRRLGNEHEESNSPLATPCVWPTLDPWTVETTTSSLTITTSTKDGTTVTVQLR

SEQ ID NO: 8 - DNA, 165 bp, synthetic construct
  source 1..165: mol_type = other DNA
caatgcaagcgccgtctgggtaacgagcacgaggaatccaactccccgctggctactccgtgtgtttggccgactctggacccgtggaccgtggaaactaccacttcttccctgactatcactacctccaccaaggacggcaccactgttactgttcaactgcgt

SEQ ID NO: 9 - protein, 58 aa, synthetic construct
MHGRHVTLKDIVLDLQPPDPVGLHCYEQLVDSSEDEVDEVDGQDSQPLKQHFQIVTCC

SEQ ID NO: 10 - DNA, 174 bp, synthetic construct
  source 1..174: mol_type = other DNA
atgcacggcaggcacgtcaccctcaaggatatcgtcctggatctgcaaccccctgaccctgtgggactgcattgctatgagcaactggtggattccagcgaagacgaagtggatgaggtggacggacaggatagccaacccctcaagcaacactttcagattgtgacatgctgt

SEQ ID NO: 11 - protein, 150 aa, synthetic construct
MESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 12 - DNA, 450 bp, synthetic construct
  source 1..450: mol_type = other DNA
atggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattctgacaaaggctagatttatcaaactgaattgcacaagaaaaggcaggtgcctccactgttggacaacctgtatggaagacatgctgcct

SEQ ID NO: 13 - protein, 267 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQERHRHLFDLISSTWHWASSKAPHKHAIVTVTYDSEEQRQQFLDVVKIPPTISHKLGFMSLHLL

SEQ ID NO: 14 - DNA, 1104 bp, synthetic construct; identical to SEQ ID NO: 2
  source 1..1104: mol_type = other DNA

SEQ ID NO: 15 - protein, 40 aa, synthetic construct
MADDSALHKKYPFLNLLHTPPLATPCVWPTLDPWTVETTT

SEQ ID NO: 16 - DNA, 120 bp, synthetic construct
  source 1..120: mol_type = other DNA
atggcagacgattctgcactgcataaaaagtacccattcctgaacctgctgcataccccaccgctggctactccgtgtgtttggccgactctggacccgtggaccgtggaaactaccact

SEQ ID NO: 17 - protein, 40 aa, synthetic construct; identical to SEQ ID NO: 15

SEQ ID NO: 18 - DNA, 60 bp, synthetic construct
  source 1..60: mol_type = other DNA
ccgctcgcaacaccatgcgtttggcccacactcgatccctggactgtcgagacaaccaca

SEQ ID NO: 19 - DNA, 60 bp, synthetic construct
  source 1..60: mol_type = other DNA
ccgctggctactccgtgtgtttggccgactctggacccgtggaccgtggaaactaccact

SEQ ID NO: 20 - protein, 120 aa, synthetic construct
MESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHI

SEQ ID NO: 21 - DNA, 360 bp, synthetic construct
  source 1..360: mol_type = other DNA
atggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacatt

SEQ ID NO: 22 - protein, 62 aa, synthetic construct
MHGRHVTLKDIVLDLQPPDPVGLHCYEQLVDSSEDEVDEIREVQQLLLGTLNIVCPICAPKT

SEQ ID NO: 23 - DNA, 186 bp, synthetic construct
  source 1..186: mol_type = other DNA
atgcacggcaggcacgtcaccctcaaggatatcgtcctggatctgcaaccccctgaccctgtgggactgcattgctatgagcaactggtggattccagcgaagacgaagtggatgagattagagaagtgcaacagctcctgctcggcacactgaatatcgtctgccctatctgtgcccctaagaca

SEQ ID NO: 24 - protein, 205 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQE

SEQ ID NO: 25 - DNA, 615 bp, synthetic construct
  source 1..615: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagag

SEQ ID NO: 26 - protein, 20 aa, synthetic construct
MADDSALHKKYPFLNLLHTP

SEQ ID NO: 27 - DNA, 60 bp, synthetic construct
  source 1..60: mol_type = other DNA
atggcagacgattctgcactgcataaaaagtacccattcctgaacctgctgcatacccca

SEQ ID NO: 28 - protein, 62 aa, synthetic construct
RHRHLFDLISSTWHWASSKAPHKHAIVTVTYDSEEQRQQFLDVVKIPPTISHKLGFMSLHLL

SEQ ID NO: 29 - DNA, 186 bp, synthetic construct
  source 1..186: mol_type = other DNA
agacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctc

SEQ ID NO: 30 - protein, 22 aa, synthetic construct
ALHKKYPFLNLLHTPPHRPPPL

SEQ ID NO: 31 - DNA, 66 bp, synthetic construct
  source 1..66: mol_type = other DNA
gcactgcataaaaagtacccattcctgaacctgctgcataccccaccgcatcgcccaccgccactg

SEQ ID NO: 32 - protein, 23 aa, synthetic construct
PLATPCVWPTLDPWTVETTTSSL

SEQ ID NO: 33 - DNA, 69 bp, synthetic construct
  source 1..69: mol_type = other DNA
ccgctggctactccgtgtgtttggccgactctggacccgtggaccgtggaaactaccacttcttccctg

SEQ ID NO: 34 - protein, 10 aa, synthetic construct
EAAAKEAAAK

SEQ ID NO: 35 - DNA, 30 bp, synthetic construct
  source 1..30: mol_type = other DNA
gaggctgccgctaaggaagctgccgccaaa

SEQ ID NO: 36 - DNA, 30 bp, synthetic construct
  source 1..30: mol_type = other DNA
gaggctgccgccaaggaggccgccgctaag

SEQ ID NO: 37 - DNA, 30 bp, synthetic construct
  source 1..30: mol_type = other DNA
gaggccgcggctaaagaggcggccgcgaaa

SEQ ID NO: 38 - DNA, 30 bp, synthetic construct
  source 1..30: mol_type = other DNA
gaggcagccgcaaaggaggctgccgcaaaa

SEQ ID NO: 39 - DNA, 30 bp, synthetic construct
  source 1..30: mol_type = other DNA
gaggcagccgcaaaggaggccgcagctaag

SEQ ID NO: 40 - protein, 15 aa, synthetic construct
TAEIYSYAYKQLKVL

SEQ ID NO: 41 - DNA, 45 bp, synthetic construct
  source 1..45: mol_type = other DNA
acagccgaaatctatagctatgcctataagcaactgaaagtgctc

SEQ ID NO: 42 - protein, 15 aa, synthetic construct
TAEIYAYAYKNLKVV

SEQ ID NO: 43 - DNA, 45 bp, synthetic construct
  source 1..45: mol_type = other DNA
acagccgaaatctatgcctatgcctataagaatctgaaagtggtc

SEQ ID NO: 44 - DNA, 45 bp, synthetic construct
  source 1..45: mol_type = other DNA
accgctgagatttacgcttacgcttacaaaaacctcaaggtcgtg

SEQ ID NO: 45 - protein, 15 aa, synthetic construct
HCYEQLEDSSEDEVD

SEQ ID NO: 46 - DNA, 45 bp, synthetic construct
  source 1..45: mol_type = other DNA
cattgctatgagcaactggaagactccagcgaagacgaagtggat

SEQ ID NO: 47 - DNA, 45 bp, synthetic construct
  source 1..45: mol_type = other DNA
cactgttacgaacagctggaggatagctccgaggatgaggtggac

SEQ ID NO: 48 - protein, 9 aa, synthetic construct
KLPQLCTEV

SEQ ID NO: 49 - DNA, 27 bp, synthetic construct
  source 1..27: mol_type = other DNA
aagctcccccaactgtgtaccgaagtg

SEQ ID NO: 50 - protein, 10 aa, synthetic construct
QLYNKPLCDV

SEQ ID NO: 51 - DNA, 30 bp, synthetic construct
  source 1..30: mol_type = other DNA
cagctctacaataagcctctgtgtgacgtg

SEQ ID NO: 52 - protein, 10 aa, synthetic construct
RTLEDLLMGV

SEQ ID NO: 53 - DNA, 30 bp, synthetic construct
  source 1..30: mol_type = other DNA
agaacactggaagacctcctgatgggcgtc

SEQ ID NO: 54 - DNA, 30 bp, synthetic construct
  source 1..30: mol_type = other DNA
agaaccctggaggatcttctcatgggggtg

SEQ ID NO: 55 - DNA, 30 bp, synthetic construct
  source 1..30: mol_type = other DNA
agaacgctagaagatcttctcatgggtgtg

SEQ ID NO: 56 - DNA, 30 bp, synthetic construct
  source 1..30: mol_type = other DNA
cgaacactggaagacttgctgatgggagtc

SEQ ID NO: 57 - protein, 9 aa, synthetic construct
VSQTSKLTR

SEQ ID NO: 58 - DNA, 27 bp, synthetic construct
  source 1..27: mol_type = other DNA
gtgtcccagacaagcaaactgacaaga

SEQ ID NO: 59 - DNA, 27 bp, synthetic construct
  source 1..27: mol_type = other DNA
gtaagccagacaagcaagcttaccaga

SEQ ID NO: 60 - DNA, 27 bp, synthetic construct
  source 1..27: mol_type = other DNA
gttagccaaaccagtaagttgaccagg

SEQ ID NO: 61 - DNA, 27 bp, synthetic construct
  source 1..27: mol_type = other DNA
gtatcccagactagcaagctcaccagg

SEQ ID NO: 62 - DNA, 27 bp, synthetic construct
  source 1..27: mol_type = other DNA
gttagtcagaccagtaagttgacaagg

SEQ ID NO: 63 - DNA, 27 bp, synthetic construct
  source 1..27: mol_type = other DNA
gtgtcccagacatcaaagctgactaga

SEQ ID NO: 64 - DNA, 27 bp, synthetic construct
  source 1..27: mol_type = other DNA
gtctctcagacctctaaactcacccga

SEQ ID NO: 65 - DNA, 27 bp, synthetic construct
  source 1..27: mol_type = other DNA
gtgtcccagacaagcaaactgacaaga

SEQ ID NO: 66 - protein, 766 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLEAAAKEAAAKMADDSALHKKYPFLNLLHTPPHRPPPLCPQAPRKTQCKRRLGNEHEESNSPLATPCEAAAKEAAAKQCTETDIREVQQLLLGTLNIVCPICAPKTEAAAKEAAAKQCKRRLGNEHEESNSPLATPCVWPTLDPWTVETTTSSLTITTSTKDGTTVTVQLREAAAKEAAAKMHGRHVTLKDIVLDLQPPDPVGLHCYEQLVDSSEDEVDEVDGQDSQPLKQHFQIVTCCEAAAKEAAAKMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 67 - DNA, 2298 bp, synthetic construct
  source 1..2298: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctcgaggctgccgctaaggaagctgccgccaaaatggcagacgattctgcactgcataaaaagtacccattcctgaacctgctgcataccccaccgcatcgcccaccgccactgtgtccgcaagctccacgcaagacccaatgcaagcgccgtctgggtaacgagcacgaggaatccaactccccgctggctactccgtgtgaggctgccgccaaggaggccgccgctaagcaatgcacagagacagacattagagaagtgcaacagctcctgctcggcacactgaatatcgtctgccctatctgtgcccctaagacagaggccgcggctaaagaggcggccgcgaaacaatgcaagcgccgtctgggtaacgagcacgaggaatccaactccccgctggctactccgtgtgtttggccgactctggacccgtggaccgtggaaactaccacttcttccctgactatcactacctccaccaaggacggcaccactgttactgttcaactgcgtgaggcagccgcaaaggaggctgccgcaaaaatgcacggcaggcacgtcaccctcaaggatatcgtcctggatctgcaaccccctgaccctgtgggactgcattgctatgagcaactggtggattccagcgaagacgaagtggatgaggtggacggacaggatagccaacccctcaagcaacactttcagattgtgacatgctgtgaggcagccgcaaaggaggccgcagctaagatggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattctgacaaaggctagatttatcaaactgaattgcacaagaaaaggcaggtgcctccactgttggacaacctgtatggaagacatgctgcct

SEQ ID NO: 68 - protein, 791 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 69 - DNA, 2373 bp, synthetic construct
  source 1..2373: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctccattgctatgagcaactggaagactccagcgaagacgaagtggatatggcagacgattctgcactgcataaaaagtacccattcctgaacctgctgcataccccaccgcatcgcccaccgccactgtgtccgcaagctccacgcaagacccaatgcaagcgccgtctgggtaacgagcacgaggaatccaactccccgctggctactccgtgtacagccgaaatctatgcctatgcctataagaatctgaaagtggtccaatgcacagagacagacattagagaagtgcaacagctcctgctcggcacactgaatatcgtctgccctatctgtgcccctaagacaacagccgaaatctatagctatgcctataagcaactgaaagtgctccaatgcaagcgccgtctgggtaacgagcacgaggaatccaactccccgctggctactccgtgtgtttggccgactctggacccgtggaccgtggaaactaccacttcttccctgactatcactacctccaccaaggacggcaccactgttactgttcaactgcgtaccgctgagatttacgcttacgcttacaaaaacctcaaggtcgtgatgcacggcaggcacgtcaccctcaaggatatcgtcctggatctgcaaccccctgaccctgtgggactgcattgctatgagcaactggtggattccagcgaagacgaagtggatgaggtggacggacaggatagccaacccctcaagcaacactttcagattgtgacatgctgtcactgttacgaacagctggaggatagctccgaggatgaggtggacatggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattctgacaaaggctagatttatcaaactgaattgcacaagaaaaggcaggtgcctccactgttggacaacctgtatggaagacatgctgcct

SEQ ID NO: 70 - protein, 599 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQERHRHLFDLISSTWHWASSKAPHKHAIVTVTYDSEEQRQQFLDVVKIPPTISHKLGFMSLHLLEAAAKEAAAKTAEIYAYAYKNLKVVEAAAKEAAAKMADDSALHKKYPFLNLLHTPPLATPCVWPTLDPWTVETTTEAAAKEAAAKHCYEQLEDSSEDEVDEAAAKEAAAKPLATPCVWPTLDPWTVETTTEAAAKEAAAKMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHIEAAAKEAAAKMHGRHVTLKDIVLDLQPPDPVGLHCYEQLVDSSEDEVDEIREVQQLLLGTLNIVCPICAPKT

SEQ ID NO: 71 - DNA, 1797 bp, synthetic construct
  source 1..1797: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagagagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctcgaggctgccgctaaggaagctgccgccaaaacagccgaaatctatgcctatgcctataagaatctgaaagtggtcgaggctgccgccaaggaggccgccgctaagatggcagacgattctgcactgcataaaaagtacccattcctgaacctgctgcataccccaccgctggctactccgtgtgtttggccgactctggacccgtggaccgtggaaactaccactgaggccgcggctaaagaggcggccgcgaaacattgctatgagcaactggaagactccagcgaagacgaagtggatgaggcagccgcaaaggaggctgccgcaaaaccgctcgcaacaccatgcgtttggcccacactcgatccctggactgtcgagacaaccacagaggcagccgcaaaggaggccgcagctaagatggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattgaggctgccgctaaggaagctgccgccaaaatgcacggcaggcacgtcaccctcaaggatatcgtcctggatctgcaaccccctgaccctgtgggactgcattgctatgagcaactggtggattccagcgaagacgaagtggatgagattagagaagtgcaacagctcctgctcggcacactgaatatcgtctgccctatctgtgcccctaagaca

SEQ ID NO: 72 - protein, 411 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQERTLEDLLMGVTAEIYAYAYKNLKVVRTLEDLLMGVMADDSALHKKYPFLNLLHTPKLPQLCTEVHCYEQLEDSSEDEVDQLYNKPLCDVPLATPCVWPTLDPWTVETTTRTLEDLLMGVTAEIYSYAYKQLKVLRTLEDLLMGVRHRHLFDLISSTWHWASSKAPHKHAIVTVTYDSEEQRQQFLDVVKIPPTISHKLGFMSLHLL

SEQ ID NO: 73 - DNA, 1233 bp, synthetic construct
  source 1..1233: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagagagaacactggaagacctcctgatgggcgtcacagccgaaatctatgcctatgcctataagaatctgaaagtggtcagaaccctggaggatcttctcatgggggtgatggcagacgattctgcactgcataaaaagtacccattcctgaacctgctgcataccccaaagctcccccaactgtgtaccgaagtgcattgctatgagcaactggaagactccagcgaagacgaagtggatcagctctacaataagcctctgtgtgacgtgccgctggctactccgtgtgtttggccgactctggacccgtggaccgtggaaactaccactcgaacactggaagacttgctgatgggagtcacagccgaaatctatagctatgcctataagcaactgaaagtgctcagaacgctagaagatcttctcatgggtgtgagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctc

SEQ ID NO: 74 - protein, 559 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLVSQTSKLTRTAEIYSYAYKQLKVLVSQTSKLTRALHKKYPFLNLLHTPPHRPPPLVSQTSKLTRPLATPCVWPTLDPWTVETTTSSLVSQTSKLTRHCYEQLEDSSEDEVDVSQTSKLTRTAEIYAYAYKNLKVVVSQTSKLTRKLPQLCTEVVSQTSKLTRQLYNKPLCDVVSQTSKLTRRTLEDLLMGV

SEQ ID NO: 75 - DNA, 1677 bp, synthetic construct
  source 1..1677: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctcgtgtcccagacaagcaaactgacaagaacagccgaaatctatagctatgcctataagcaactgaaagtgctcgtaagccagacaagcaagcttaccagagcactgcataaaaagtacccattcctgaacctgctgcataccccaccgcatcgcccaccgccactggttagccaaaccagtaagttgaccaggccgctggctactccgtgtgtttggccgactctggacccgtggaccgtggaaactaccacttcttccctggtatcccagactagcaagctcaccaggcattgctatgagcaactggaagactccagcgaagacgaagtggatgttagtcagaccagtaagttgacaaggacagccgaaatctatgcctatgcctataagaatctgaaagtggtcgtgtcccagacatcaaagctgactagaaagctcccccaactgtgtaccgaagtggtctctcagacctctaaactcacccgacagctctacaataagcctctgtgtgacgtggtgtcccagacaagcaaactgacaagaagaacactggaagacctcctgatgggcgtc

SEQ ID NO: 76 - protein, 5 aa, synthetic construct
EAAAK

SEQ ID NO: 77 - DNA, 15 bp, synthetic construct
  source 1..15: mol_type = other DNA
gaggctgccgctaag

SEQ ID NO: 78 - DNA, 15 bp, synthetic construct
  source 1..15: mol_type = other DNA
gaggctgccgccaag

SEQ ID NO: 79 - DNA, 15 bp, synthetic construct
  source 1..15: mol_type = other DNA
gaggccgcggctaaa

SEQ ID NO: 80 - DNA, 15 bp, synthetic construct
  source 1..15: mol_type = other DNA
gaggcagccgcaaag

SEQ ID NO: 81 - DNA, 15 bp, synthetic construct
  source 1..15: mol_type = other DNA
gaggcagccgcaaag

SEQ ID NO: 82 - DNA, 458 bp, synthetic construct
  source 1..458: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgac

SEQ ID NO: 83 - DNA, 181 bp, synthetic construct
  source 1..181: mol_type = other DNA
ccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgc

SEQ ID NO: 84 - DNA, 2376 bp, synthetic construct
  source 1..2376: mol_type = other DNA
tcaaggcagcatgtcttccatacaggttgtccaacagtggaggcacctgccttttcttgtgcaattcagtttgataaatctagcctttgtcagaatgtgtttcactttctcgacctcacactgaggcttatggcagaggtaacatctaatcagcacgtcgaggatatcctgtttggtttcctcttccactgtggtagcgtatccggcatagtcaaagtgagcgtattggttaatctttccgtgaaactccaggcaacaggcacaggcagcgtaagggtatccgcccctgaagagcactttcagatgcttataggcatagctatagatttcggctgtggtgagggcattcttacagaagacgcagttgatttgcagtgtgtgcatggagaggttaaaggttttgcagagctggtcgattgtggtagcgcttgtgctggcgttagcgctttccatgtccacctcatcctcggagctatcctccagctgttcgtaacagtgacagcatgtcacaatctgaaagtgttgcttgaggggttggctatcctgtccgtccacctcatccacttcgtcttcgctggaatccaccagttgctcatagcaatgcagtcccacagggtcagggggttgcagatccaggacgatatccttgagggtgacgtgcctgccgtgcatcacgaccttgaggtttttgtaagcgtaagcgtaaatctcagcggtacgcagttgaacagtaacagtggtgccgtccttggtggaggtagtgatagtcagggaagaagtggtagtttccacggtccacgggtccagagtcggccaaacacacggagtagccagcggggagttggattcctcgtgctcgttacccagacggcgcttgcattggagcactttcagttgcttataggcatagctatagatttcggctgttgtcttaggggcacagatagggcagacgatattcagtgtgccgagcaggagctgttgcacttctctaatgtctgtctctgtgcattggaccactttcagattcttataggcataggcatagatttcggctgtacacggagtagccagcggggagttggattcctcgtgctcgttacccagacggcgcttgcattgggtcttgcgtggagcttgcggacacagtggcggtgggcgatgcggtggggtatgcagcaggttcaggaatgggtactttttatgcagtgcagaatcgtctgccatatccacttcgtcttcgctggagtcttccagttgctcatagcaatggagcagatgcagggacataaagccgagcttatggctaatggtagggggaatcttgaccacatccagaaactgttgtctttgctcttcgctatcgtatgtcactgtcacaatggcgtgcttatggggagccttggagctggcccaatgccatgtgctggagatgaggtcaaacagatgtctatgtctatcgttgagtctatatctaaagcatttcagacagttggactctccctgaaactggacgataggggtagcgctggagttggagttatttctcctctgatgctgatcgtgattgtttgtgatgaggttatggtttccgctatccacagggccaatgtgagccacacacagagcgttacagggggactgttgcactcctctagctctttttctagggggtgtctggacggcatcctctttggtggagctggacacgagggtggaggtctgggcaggggtataggttgtgctttcgggaatgctgacctcttgggttgtgctggacacggaggcagggctacagatgacggtggagccatagcagacctcccagtgtttggtggagccatacttttcggcttccttgacgaaattcacatagtatgtcttaaactgtccgcatgtgtaatagattcccttagcgtccaccatggagtggaccttgacccatgtgtcattgtcttgcacatacacatcggtccacacgacgtaatccattgtgttattggcacagccatcgaatttcacttccactgtctttcctcttttcttaaagcacctcttagggggtgtctgccacatagcgtaggaggtttcctggagggtccagggttccatggagtattcggtcctcaggaggctttccaggtgcatttgcatttcgatagcgttatggcctttggcttcgctgaccttgaggggaggcacgacctgcattccaatgtgggacagtcccatctgcttggccttatacaggagcacggaggcatgtctcatgcacttccaatgcaggacgtgtttgtggaggtctgtgctattctcttcggccagctccagcagctgctcggcgcaagcgtcgagtcttttggcaatggcttccat

SEQ ID NO: 85 - DNA, 920 bp, synthetic construct
  source 1..920: mol_type = other DNA
ggtggcgggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactag

SEQ ID NO: 86 - DNA, 5294 bp, synthetic construct
  source 1..5294: mol_type = other DNA
ctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtctccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcaccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggatcaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaagcaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgttgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgcgagaaggcgttgagcccgccctcgttccagacccggctgtagaccacgcccccctcggcgttgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgtcgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgggcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggttttccagcatgaggcggtggtatccgtagatgtacctggacatccaggtgatgccggcggcggtggtggtggcgcgcgcgtagtcgcggacccggttccagatgtttcgcaggggcgagaagtgttccat

SEQ ID NO: 87 - DNA, 3040 bp, synthetic construct
  source 1..3040: mol_type = other DNA
atgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtgaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgttcatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgagaagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaa

SEQ ID NO: 88 - DNA, 4025 bp, synthetic construct
  source 1..4025: mol_type = other DNA
atgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcactcagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgccgtggccccgcgggcacgaaggcgcgcggccgccgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttga

SEQ ID NO: 89 - DNA, 4378 bp, synthetic construct
  source 1..4378: mol_type = other DNA
atggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagaggaaaatcaggtggtcgctgcagatgatgaacttgaagatgaagaagcgcaagctcaagaggacgccccagctaaaaaaattcatgtatatgcccaggcgcctcttgctggcgaaaagattaccaaggatggtttgcaaataggtactgaagttgtaggagatacatctaaggacacttttgcagacaaaacattccaacccgaacctcagataggcgagtctcagtggaacgaggctgatgccacagtagcaggaggcagagtcttgaaaaaaaccacccctatgagaccttgctatggatcctatgccaggcctacaaatgccaacgggggtcaaggaattatggttgccaatgaacaaggagtgttggagtctaaagtggagatgcaatttttttctaacactacaacccttaatgcgcgggatggagctggcaatcccgaaccaaaggtggtgttgtacagtgaagatgtccacttggaatctcctgacactcatttgtcttacaagcccaaaaaggatgatgttaatgctaaaattatgttgggtcagcaagctatggctaacaggcccaacctcattgcttttagagataatttcattggactcatgtactacaacagcactggtaacatgggagtgctggcgggtcaggcctctcagttgaatgccgtggtggacctgcaggatagaaacacagaactgtcatatcagcttatgcttgattccattggggatagatccagatacttctccatgtggaaccaggcagtggatagctatgacccagatgtcagaatcattgaaaaccatggtgtcgaggacgagctacccaactactgcttccctctgggcggcataggaattactgatacttatcaagggatcaaaaataccaatggcaatggtcagtggaccaaagatgatcagttcgcggaccgtaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaagaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaacaaaacatgcaagacggaaaatga

SEQ ID NO: 90 - DNA, 1638 bp, synthetic construct
  source 1..1638: mol_type = other DNA
ctagaaatcgaaagggttctgccgcgcgtcgccgtgcgccacgggcagagacacgttgcgatactggaagcggctcgcccacttgaactcgggcaccaccatgcggggcagtggctcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattacgcacgctggccagcaggctctcgtcgctgatcatgtcgctgtccagatcctccgcgttgctcagggcgaatggggtcatcttgcagacctgcctgcccaggaaaggcggcagcccgggcttgccgttgcagtcgcagcgcaggggcatcagcaggtgcccgtggcccgtctgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtcccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagctccgcctgctcttcttcgtcttcgctgtctaccactatctctggggaagggcttctccgctctgcggcggtgcgcttcttttttttcttgggagcagccgtgacggagtccgccacggcgacggaggtcgagggcgtggggctgggggtgcgcggtaccagggcctcgtcgccctcggactcttcctctgactccaggcggcggcggagacgcttctttgggggcgcgcgcgtcagcggcggcggagacggggacggggacggggacgggacgccctccacagggggtggtcttcgcgcagacccgcggccgcgctcgggggtcttttcgagctggtcttggtcccgactggccat

SEQ ID NO: 91 - DNA, 3845 bp, synthetic construct
  source 1..3845: mol_type = other DNA
atgcaagtcgagaaggaggagagcttaaccaccccctctgagaccgccgatgcgcccgccgtcgccgtcgcccccgctgccgccgacgcgcccgccacaccgagcgacacccccgcggaccccccagccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccatcgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtgcccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtgccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctcaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgcggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagacctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagattttttgctgagaaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgagaaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcgcagctcgcgggcggctgtcgtcacagggtgcggcctcctcgccagggtataactcacctggagatccgaggcagaggtattcagctcaacgacgagtcggtgagctcctcgctcggtctaagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactga

SEQ ID NO: 92 - DNA, 3888 bp, synthetic construct
  source 1..3888: mol_type = other DNA
atgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacaagagttaacacttgtggggtttctggtgtatgtgacgctggtggtggctcttttgattaaggcttttccttccatgtctgaactctccctcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaacgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcatcgatgaaaagaaaatgccttaaacgctttcacgcttgattgctaacaccgggtttttatccgcagaatgattggaatcaccctactaatcacctccctccttgcgattgcccatgggttggaacgaatcgaagcccctgtgggggccaatgttaccctggtggggcctgtcggcaatgctacattaatgtgggaaaaatatactaaaaatcaatgggtctcttactgcactaacaaaaacagccacaagcccagagccatctgcgatgggcaaaatctaaccttgattgatgttcaaatgctggatgcgggctactattatgggcagctgggtacaatgattaattactggagaccccacaaagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgagaccaccgagatctgcttctgcaaatgctctgacgcctttgctgaggatttggaagaccacgaggaagatgagcatgacttcgcagatgcatgccaggcatcagaggcagaagcgctgccggtggccctcaaacagtatgcagacccccacaccacccccaaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccagaagttcgccctcatgataggcgaacaacccatcaccgtgacccagcactccgtggagacagaaggctgcatacacgctccctgtaggggcgctgactgcctctacaccttgatcaaaaccctctgcggtctcagagacctcatcccttttaattaa

SEQ ID NO: 93 - DNA, 1743 bp, synthetic construct
  source 1..1743: mol_type = other DNA
atgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagacatgcaagcgcccattagctctcgagatggaaaactggctctaacagtggcggcccccctaactgtggtcgagggtatcaatgctttggcagtagccacaggtaagggtattgggctaaatgaaaccaacacacacctgcaggcaaaactggtcgcacccctaggctttgataccaacggcaacattaagctaagcgttgcaggaggcatgaggctaaacaataacacactgatactagatgtaaactacccatttgaggctcaaggccaactgagcctaagagtgggctcgggcccactatatgtagattctagtagtcataacctaaccattagatgccttaggggattgtatataacatcttctaacaaccaaaacggtctagaagccaacattaaactaacaagaggccttgtgtatgacggaaatgccatagcagttaatgttggcaaagggctggaatacagccctactgacacaacagaaaaacctatacagactaaaataggtctaggcatggagtatgataccgagggagccatgatgacaaaactaggctctggactaagctttgacaattcaggagccattgtagtgggaaacaaaaatgatgacaggcttactttgtggaccacaccggacccatcgcccaactgtcagatctactctgaaaaagatgctaaactaaccttggtactgactaaatgtggcagtcaggttgtaggcacagtatctattgccgctcttaaaggtagcctcgtgccaatcactagtgcaatcagtgtggttcaggtatacctaaggtttgatgaaaatggggtactaatgagtaactcttcacttaatggcgaatactggaattttagaaacggagactcaactaatggcacaccatatacaaacgcagtgggtttcatgcctaatctactggcctatcctaaaggtcaaactacaactgcaaaaagtaacattgtcagccaggtctacatgaatggggacgatactaaacccatgacatttacaatcaacttcaatggccttagtgaaacaggggatacccctgttagtaaatattccatgacattctcatggaggtggccaaatggaagctacatagggcacaattttgtaacaaactcctttaccttctcctacatcgcccaagaataa

SEQ ID NO: 94 - DNA, 84 bp, synthetic construct
  source 1..84: mol_type = other DNA
tcctacgtcacccgccccgcctctccccgcccacctcattatcatattggccacaatccaaaataaggtatattattgatgatg

SEQ ID NO: 95 - DNA, 278 bp, synthetic construct
  source 1..278: mol_type = other DNA
caattgacggccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagggtaccaacgcgtcgtaactataacggtcctaaggtagcg

SEQ ID NO: 96 - DNA, 365 bp, synthetic construct
  source 1..365: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 97 - DNA, 205 bp, synthetic construct
  source 1..205: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 98 - DNA, 42 bp, synthetic construct
  source 1..42: mol_type = other DNA
tccctatcagtgatagagatctccctatcagtgatagagatc

SEQ ID NO: 99 - DNA, 295 bp, synthetic construct
  source 1..295: mol_type = other DNA
gtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagcc

SEQ ID NO: 100-101 - skipped (2 entries)

SEQ ID NO: 102 - DNA, 2376 bp, synthetic construct
  source 1..2376: mol_type = other DNA
atggaagccattgccaaaagactcgacgcttgcgccgagcagctgctggagctggccgaagagaatagcacagacctccacaaacacgtcctgcattggaagtgcatgagacatgcctccgtgctcctgtataaggccaagcagatgggactgtcccacattggaatgcaggtcgtgcctcccctcaaggtcagcgaagccaaaggccataacgctatcgaaatgcaaatgcacctggaaagcctcctgaggaccgaatactccatggaaccctggaccctccaggaaacctcctacgctatgtggcagacaccccctaagaggtgctttaagaaaagaggaaagacagtggaagtgaaattcgatggctgtgccaataacacaatggattacgtcgtgtggaccgatgtgtatgtgcaagacaatgacacatgggtcaaggtccactccatggtggacgctaagggaatctattacacatgcggacagtttaagacatactatgtgaatttcgtcaaggaagccgaaaagtatggctccaccaaacactgggaggtctgctatggctccaccgtcatctgtagccctgcctccgtgtccagcacaacccaagaggtcagcattcccgaaagcacaacctatacccctgcccagacctccaccctcgtgtccagctccaccaaagaggatgccgtccagacaccccctagaaaaagagctagaggagtgcaacagtccccctgtaacgctctgtgtgtggctcacattggccctgtggatagcggaaaccataacctcatcacaaacaatcacgatcagcatcagaggagaaataactccaactccagcgctacccctatcgtccagtttcagggagagtccaactgtctgaaatgctttagatatagactcaacgatagacatagacatctgtttgacctcatctccagcacatggcattgggccagctccaaggctccccataagcacgccattgtgacagtgacatacgatagcgaagagcaaagacaacagtttctggatgtggtcaagattccccctaccattagccataagctcggctttatgtccctgcatctgctccattgctatgagcaactggaagactccagcgaagacgaagtggatatggcagacgattctgcactgcataaaaagtacccattcctgaacctgctgcataccccaccgcatcgcccaccgccactgtgtccgcaagctccacgcaagacccaatgcaagcgccgtctgggtaacgagcacgaggaatccaactccccgctggctactccgtgtacagccgaaatctatgcctatgcctataagaatctgaaagtggtccaatgcacagagacagacattagagaagtgcaacagctcctgctcggcacactgaatatcgtctgccctatctgtgcccctaagacaacagccgaaatctatagctatgcctataagcaactgaaagtgctccaatgcaagcgccgtctgggtaacgagcacgaggaatccaactccccgctggctactccgtgtgtttggccgactctggacccgtggaccgtggaaactaccacttcttccctgactatcactacctccaccaaggacggcaccactgttactgttcaactgcgtaccgctgagatttacgcttacgcttacaaaaacctcaaggtcgtgatgcacggcaggcacgtcaccctcaaggatatcgtcctggatctgcaaccccctgaccctgtgggactgcattgctatgagcaactggtggattccagcgaagacgaagtggatgaggtggacggacaggatagccaacccctcaagcaacactttcagattgtgacatgctgtcactgttacgaacagctggaggatagctccgaggatgaggtggacatggaaagcgctaacgccagcacaagcgctaccacaatcgaccagctctgcaaaacctttaacctctccatgcacacactgcaaatcaactgcgtcttctgtaagaatgccctcaccacagccgaaatctatagctatgcctataagcatctgaaagtgctcttcaggggcggatacccttacgctgcctgtgcctgttgcctggagtttcacggaaagattaaccaatacgctcactttgactatgccggatacgctaccacagtggaagaggaaaccaaacaggatatcctcgacgtgctgattagatgttacctctgccataagcctcagtgtgaggtcgagaaagtgaaacacattctgacaaaggctagatttatcaaactgaattgcacaagaaaaggcaggtgcctccactgttggacaacctgtatggaagacatgctgccttga

SEQ ID NO: 103 - DNA, 49 bp, synthetic construct
  source 1..49: mol_type = other DNA
gcggccgctctcgagtctagctagtctagactagctagaaagatccggg

SEQ ID NO: 104 - DNA, 132 bp, synthetic construct
  source 1..132: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 105 - protein, 368 aa, synthetic construct; identical to SEQ ID NO: 1

SEQ ID NO: 106 - protein, 15 aa, synthetic construct
HCYEQLEDSSEDEVD

SEQ ID NO: 107 - protein, 56 aa, synthetic construct; identical to SEQ ID NO: 3

SEQ ID NO: 108 - protein, 15 aa, synthetic construct
TAEIYAYAYKNLKVV

SEQ ID NO: 109 - protein, 29 aa, synthetic construct
QCTETDIREVQQLLLGTLNIVCPICAPKT

SEQ ID NO: 110 - protein, 15 aa, synthetic construct
TAEIYSYAYKQLKVL

SEQ ID NO: 111 - protein, 55 aa, synthetic construct; identical to SEQ ID NO: 7

SEQ ID NO: 112 - protein, 15 aa, synthetic construct
TAEIYAYAYKNLKVV

SEQ ID NO: 113 - protein, 58 aa, synthetic construct; identical to SEQ ID NO: 9

SEQ ID NO: 114 - protein, 15 aa, synthetic construct
HCYEQLEDSSEDEVD

SEQ ID NO: 115 - protein, 150 aa, synthetic construct; identical to SEQ ID NO: 11

SEQ ID NO: 116 - DNA, 2373 bp, synthetic construct; identical to SEQ ID NO: 69
  source 1..2373: mol_type = other DNA

SEQ ID NO: 117 - protein, 791 aa, synthetic construct; identical to SEQ ID NO: 68

SEQ ID NO: 118 - DNA, 32767 bp, synthetic construct
  source 1..32767: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacagatcgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgctcaaggcagcatgtcttccatacaggttgtccaacagtggaggcacctgccttttcttgtgcaattcagtttgataaatctagcctttgtcagaatgtgtttcactttctcgacctcacactgaggcttatggcagaggtaacatctaatcagcacgtcgaggatatcctgtttggtttcctcttccactgtggtagcgtatccggcatagtcaaagtgagcgtattggttaatctttccgtgaaactccaggcaacaggcacaggcagcgtaagggtatccgcccctgaagagcactttcagatgcttataggcatagctatagatttcggctgtggtgagggcattcttacagaagacgcagttgatttgcagtgtgtgcatggagaggttaaaggttttgcagagctggtcgattgtggtagcgcttgtgctggcgttagcgctttccatgtccacctcatcctcggagctatcctccagctgttcgtaacagtgacagcatgtcacaatctgaaagtgttgcttgaggggttggctatcctgtccgtccacctcatccacttcgtcttcgctggaatccaccagttgctcatagcaatgcagtcccacagggtcagggggttgcagatccaggacgatatccttgagggtgacgtgcctgccgtgcatcacgaccttgaggtttttgtaagcgtaagcgtaaatctcagcggtacgcagttgaacagtaacagtggtgccgtccttggtggaggtagtgatagtcagggaagaagtggtagtttccacggtccacgggtccagagtcggccaaacacacggagtagccagcggggagttggattcctcgtgctcgttacccagacggcgcttgcattggagcactttcagttgcttataggcatagctatagatttcggctgttgtcttaggggcacagatagggcagacgatattcagtgtgccgagcaggagctgttgcacttctctaatgtctgtctctgtgcattggaccactttcagattcttataggcataggcatagatttcggctgtacacggagtagccagcggggagttggattcctcgtgctcgttacccagacggcgcttgcattgggtcttgcgtggagcttgcggacacagtggcggtgggcgatgcggtggggtatgcagcaggttcaggaatgggtactttttatgcagtgcagaatcgtctgccatatccacttcgtcttcgctggagtcttccagttgctcatagcaatggagcagatgcagggacataaagccgagcttatggctaatggtagggggaatcttgaccacatccagaaactgttgtctttgctcttcgctatcgtatgtcactgtcacaatggcgtgcttatggggagccttggagctggcccaatgccatgtgctggagatgaggtcaaacagatgtctatgtctatcgttgagtctatatctaaagcatttcagacagttggactctccctgaaactggacgataggggtagcgctggagttggagttatttctcctctgatgctgatcgtgattgtttgtgatgaggttatggtttccgctatccacagggccaatgtgagccacacacagagcgttacagggggactgttgcactcctctagctctttttctagggggtgtctggacggcatcctctttggtggagctggacacgagggtggaggtctgggcaggggtataggttgtgctttcgggaatgctgacctcttgggttgtgctggacacggaggcagggctacagatgacggtggagccatagcagacctcccagtgtttggtggagccatacttttcggcttccttgacgaaattcacatagtatgtcttaaactgtccgcatgtgtaatagattcccttagcgtccaccatggagtggaccttgacccatgtgtcattgtcttgcacatacacatcggtccacacgacgtaatccattgtgttattggcacagccatcgaatttcacttccactgtctttcctcttttcttaaagcacctcttagggggtgtctgccacatagcgtaggaggtttcctggagggtccagggttccatggagtattcggtcctcaggaggctttccaggtgcatttgcatttcgatagcgttatggcctttggcttcgctgaccttgaggggaggcacgacctgcattccaatgtgggacagtcccatctgcttggccttatacaggagcacggaggcatgtctcatgcacttccaatgcaggacgtgtttgtggaggtctgtgctattctcttcggccagctccagcagctgctcggcgcaagcgtcgagtcttttggcaatggcttccatggtggcgggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctggctcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtacacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacaaatcgggggcggagggtcccggactgggggatgatggtgccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagtctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtctccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcaccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggatcaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaagcaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgttgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgcgagaaggcgttgagcccgccctcgttccagacccggctgtagaccacgcccccctcggcgttgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgtcgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgggcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggttttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtgaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgttcatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgagaagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcactcagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgccgtggccccgcgggcacgaaggcgcgcggccgccgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaagtctagactctcacgctcgcttggtcctgtgactattttgtagaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagaggaaaatcaggtggtcgctgcagatgatgaacttgaagatgaagaagcgcaagctcaagaggacgccccagctaaaaaaattcatgtatatgcccaggcgcctcttgctggcgaaaagattaccaaggatggtttgcaaataggtactgaagttgtaggagatacatctaaggacacttttgcagacaaaacattccaacccgaacctcagataggcgagtctcagtggaacgaggctgatgccacagtagcaggaggcagagtcttgaaaaaaaccacccctatgagaccttgctatggatcctatgccaggcctacaaatgccaacgggggtcaaggaattatggttgccaatgaacaaggagtgttggagtctaaagtggagatgcaatttttttctaacactacaacccttaatgcgcgggatggagctggcaatcccgaaccaaaggtggtgttgtacagtgaagatgtccacttggaatctcctgacactcatttgtcttacaagcccaaaaaggatgatgttaatgctaaaattatgttgggtcagcaagctatggctaacaggcccaacctcattgcttttagagataatttcattggactcatgtactacaacagcactggtaacatgggagtgctggcgggtcaggcctctcagttgaatgccgtggtggacctgcaggatagaaacacagaactgtcatatcagcttatgcttgattccattggggatagatccagatacttctccatgtggaaccaggcagtggatagctatgacccagatgtcagaatcattgaaaaccatggtgtcgaggacgagctacccaactactgcttccctctgggcggcataggaattactgatacttatcaagggatcaaaaataccaatggcaatggtcagtggaccaaagatgatcagttcgcggaccgtaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaagaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaacaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttgaactcgggcaccaccatgcggggcagtggctcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattacgcacgctggccagcaggctctcgtcgctgatcatgtcgctgtccagatcctccgcgttgctcagggcgaatggggtcatcttgcagacctgcctgcccaggaaaggcggcagcccgggcttgccgttgcagtcgcagcgcaggggcatcagcaggtgcccgtggcccgtctgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtcccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagctccgcctgctcttcttcgtcttcgctgtctaccactatctctggggaagggcttctccgctctgcggcggtgcgcttcttttttttcttgggagcagccgtgacggagtccgccacggcgacggaggtcgagggcgtggggctgggggtgcgcggtaccagggcctcgtcgccctcggactcttcctctgactccaggcggcggcggagacgcttctttgggggcgcgcgcgtcagcggcggcggagacggggacggggacggggacgggacgccctccacagggggtggtcttcgcgcagacccgcggccgcgctcgggggtcttttcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggaccccccagccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccatcgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtgcccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtgccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctcaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgcggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagacctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagattttttgctgagaaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgagaaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcgcagctcgcgggcggctgtcgtcacagggtgcggcctcctcgccagggtataactcacctggagatccgaggcagaggtattcagctcaacgacgagtcggtgagctcctcgctcggtctaagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacaagagttaacacttgtggggtttctggtgtatgtgacgctggtggtggctcttttgattaaggcttttccttccatgtctgaactctccctcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaacgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcatcgatgaaaagaaaatgccttaaacgctttcacgcttgattgctaacaccgggtttttatccgcagaatgattggaatcaccctactaatcacctccctccttgcgattgcccatgggttggaacgaatcgaagcccctgtgggggccaatgttaccctggtggggcctgtcggcaatgctacattaatgtgggaaaaatatactaaaaatcaatgggtctcttactgcactaacaaaaacagccacaagcccagagccatctgcgatgggcaaaatctaaccttgattgatgttcaaatgctggatgcgggctactattatgggcagctgggtacaatgattaattactggagaccccacaaagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgagaccaccgagatctgcttctgcaaatgctctgacgcctttgctgaggatttggaagaccacgaggaagatgagcatgacttcgcagatgcatgccaggcatcagaggcagaagcgctgccggtggccctcaaacagtatgcagacccccacaccacccccaaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccagaagttcgccctcatgataggcgaacaacccatcaccgtgacccagcactccgtggagacagaaggctgcatacacgctccctgtaggggcgctgactgcctctacaccttgatcaaaaccctctgcggtctcagagacctcatcccttttaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgca

SEQ ID NO: 119 - DNA, 32767 bp, synthetic construct
  source 1..32767: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacgcggccgcctaggaacacccactacacccccactcaagggaacaccgtgcccagctaccctgagacagccccactcccaggaaatgcaggtgccaaccagccccagtgaggcatggcacctgagccagacacccccaaatagtcccttcccacctcctcaagcatctgcactctgggtgaacccagaaaaactggggcacctggggagacatagaagggccaggccaggaactccccaataagcaggaacagagacctgacccctgagcctcccccaccctctaaggctgctcaatgtcaaggcaggagggatctccacacacctgcactctgggtaattcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccatcaatcgattcagtttcgtatcttcattgtcatgtaggcttctatgtagttgatgaagatgtcaaactcactcatggctttgtagatgcctttctcttggagcttgttgaaggcattcttcacttgctccacggccttgctcttgttttcacagggaaggaatctgtgacagcgccgtagtctcagtctgagggttttcaggttctcccccagggagttcacatgcgccttgatgtctgggtcttggttctcagcttggggcatcacctcctccaggtaaaactggatcatctcagacaaggcttggcaacccaggtaacccttaaagtcctccagcaaggactcctttaacaacaagttgtccagctgatccttcatttgaaagaaggttttcactctgctgaaggcatctctcaggtctctaagcatgttggggaggttgcctgggaagtgggtgcagctgttctcagactgggtgccctggcctgggctggccctcaccccagtcaggaggaccaggcaacagagcagggcgctgctgtgcatggtggcgctagcggctctcaattccttttttccaaagtagaggacaagaagtgtccggcaacgaaggtacccagcaaggtttttcctgtcaggaacaaacatttgttgtaaatgtcatcctaacatcagcacacaaaagggattagcaaaaaaaaaaaaaaagagcatgttaagattactcaccttcttcgcctgcaggttctatggaggtcaaaacactcggctcgggatccgtctccagaatgagggttcactaaacgtcgacgatctctatcactgatagggagatctctatcactgatagggagagctctgcttatatagacctcgctcgtgatacgtcacttcctggtggatcacgcgcgatccaacaagtgcggcggacaggcgagcgcccgggcttgagaacggtactttcccattaatcagctatgggaaagtaccgtttaaaggtcacgttgcattagtttcaatagcccattgacgtcaatggtgggaaagtacatggcgtttcaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgcctattcaccctattgaccttatgtactgggcaaaacccatggaaagtccctaatgactcagtatacgtgccagtaatgggaaaaacccattggcttacctcccattgaccttattggtactgggcaaaacccatggaaagtcccctattgactcaatgtacttggctccaatggaactttcctgttgactcaccccctattgaccttatgtactgggcaaaacccaatggaaagtccctattgagtcagtgtacttggctccaatgggtttttcccattgactcatcccctattgaccttatgtactgggcaaaacccaatggaaagtccctattgacgcagtgtacttggctccaatgggacttttccgttgattcacctattgaccttatgtacgggcaggtacaatggtacccctgcattatggatagagaaatgttctggcacctggacttgcactggggacagcctattttgctagtttgttttgtttcgttttgttttgatggagagcgtatgttagtactatggattcacacaaaaaaccaacacacagatgtaatgaaaataaagatattttattgcggcacgcgtaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctggctcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtacacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacaaatcgggggcggagggtcccggactgggggatgatggtgccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagtctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtctccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcaccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggatcaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaagcaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgttgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgcgagaaggcgttgagcccgccctcgttccagacccggctgtagaccacgcccccctcggcgttgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgtcgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgggcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggttttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtgaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgttcatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgagaagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcactcagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgccgtggccccgcgggcacgaaggcgcgcggccgccgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaagtctagactctcacgctcgcttggtcctgtgactattttgtagaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagaggaaaatcaggtggtcgctgcagatgatgaacttgaagatgaagaagcgcaagctcaagaggacgccccagctaaaaaaattcatgtatatgcccaggcgcctcttgctggcgaaaagattaccaaggatggtttgcaaataggtactgaagttgtaggagatacatctaaggacacttttgcagacaaaacattccaacccgaacctcagataggcgagtctcagtggaacgaggctgatgccacagtagcaggaggcagagtcttgaaaaaaaccacccctatgagaccttgctatggatcctatgccaggcctacaaatgccaacgggggtcaaggaattatggttgccaatgaacaaggagtgttggagtctaaagtggagatgcaatttttttctaacactacaacccttaatgcgcgggatggagctggcaatcccgaaccaaaggtggtgttgtacagtgaagatgtccacttggaatctcctgacactcatttgtcttacaagcccaaaaaggatgatgttaatgctaaaattatgttgggtcagcaagctatggctaacaggcccaacctcattgcttttagagataatttcattggactcatgtactacaacagcactggtaacatgggagtgctggcgggtcaggcctctcagttgaatgccgtggtggacctgcaggatagaaacacagaactgtcatatcagcttatgcttgattccattggggatagatccagatacttctccatgtggaaccaggcagtggatagctatgacccagatgtcagaatcattgaaaaccatggtgtcgaggacgagctacccaactactgcttccctctgggcggcataggaattactgatacttatcaagggatcaaaaataccaatggcaatggtcagtggaccaaagatgatcagttcgcggaccgtaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaagaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaacaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttgaactcgggcaccaccatgcggggcagtggctcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattacgcacgctggccagcaggctctcgtcgctgatcatgtcgctgtccagatcctccgcgttgctcagggcgaatggggtcatcttgcagacctgcctgcccaggaaaggcggcagcccgggcttgccgttgcagtcgcagcgcaggggcatcagcaggtgcccgtggcccgtctgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtcccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagctccgcctgctcttcttcgtcttcgctgtctaccactatctctggggaagggcttctccgctctgcggcggtgcgcttcttttttttcttgggagcagccgtgacggagtccgccacggcgacggaggtcgagggcgtggggctgggggtgcgcggtaccagggcctcgtcgccctcggactcttcctctgactccaggcggcggcggagacgcttctttgggggcgcgcgcgtcagcggcggcggagacggggacggggacggggacgggacgccctccacagggggtggtcttcgcgcagacccgcggccgcgctcgggggtcttttcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggaccccccagccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccatcgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtgcccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtgccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctcaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgcggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagacctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagattttttgctgagaaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgagaaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcgcagctcgcgggcggctgtcgtcacagggtgcggcctcctcgccagggtataactcacctggagatccgaggcagaggtattcagctcaacgacgagtcggtgagctcctcgctcggtctaagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacaagagttaacacttgtggggtttctggtgtatgtgacgctggtggtggctcttttgattaaggcttttccttccatgtctgaactctccctcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaacgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcatcgatgaaaagaaaatgccttaaacgctttcacgcttgattgctaacaccgggtttttatccgcagaatgattggaatcaccctactaatcacctccctccttgcgattgcccatgggttggaacgaatcgaagcccctgtgggggccaatgttaccctggtggggcctgtcggcaatgctacattaatgtgggaaaaatatactaaaaatcaatgggtctcttactgcactaacaaaaacagccacaagcccagagccatctgcgatgggcaaaatctaaccttgattgatgttcaaatgctggatgcgggctactattatgggcagctgggtacaatgattaattactggagaccccacaaagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgagaccaccgagatctgcttctgcaaatgctctgacgcctttgctgaggatttggaagaccacgaggaagatgagcatgacttcgcagatgcatgccaggcatcagaggcagaagcgctgccggtggccctcaaacagtatgcagacccccacaccacccccaaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccagaagttcgccctcatgataggcgaacaacccatcaccgtgacccagcactccgtggagacagaaggctgcatacacgctccctgtaggggcgctgactgcctctacaccttgatcaaaaccctctgcggtctcagagacctcatcccttttaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagacatgcaagcgcccattagctctcgagatggaaaactggctctaacagtggcggcccccctaactgtggtcgagggtatcaatgctttggcagtagccacaggtaagggtattgggctaaatgaaaccaacacacacctgcaggcaaaactggtcgcacccctaggctttgataccaacggcaacattaagctaagcgttgcaggaggcatgaggctaaacaataacacactgatactagatgtaaactacccatttgaggctcaaggccaactgagcctaagagtgggctcgggcccactatatgtagattctagtagtcataacctaaccattagatgccttaggggattgtatataacatcttctaacaaccaaaacggtctagaagccaacattaaactaacaagaggccttgtgtatgacggaaatgccatagcagttaatgttggcaaagggctggaatacagccctactgacacaacagaaaaacctatacagactaaaataggtctaggcatggagtatgataccgagggagccatgatgacaaaactaggctctgg

SEQ ID NO: 120 - protein, 10 aa, unidentified
IYSYAYKHLK

SEQ ID NO: 121 - protein, 4 aa, synthetic construct
SGSG

SEQ ID NO: 122 - protein, 8 aa, unidentified
  VARIANT 2: note = V or I
  VARIANT 4: note = Any amino acid
DXEXNPGP

SEQ ID NO: 123 - protein, 4 aa, synthetic construct
RAKR

SEQ ID NO: 124 - protein, 8 aa, synthetic construct
APVKQGSG

SEQ ID NO: 125 - DNA, 12 bp, synthetic construct
  source 1..12: mol_type = other DNA
cgtgcaaagcgt

SEQ ID NO: 126 - DNA, 12 bp, synthetic construct
  source 1..12: mol_type = other DNA
agagctaagagg

SEQ ID NO: 127 - protein, 5 aa, synthetic construct
GGGGS

SEQ ID NO: 128 - protein, 27 aa, synthetic construct
  VARIANT 2..26: note = This region may encompass 2-5 EAAAK repeating units
AEAAAKEAAAKEAAAKEAAAKEAAAKA

SEQ ID NO: 129 - protein, 30 aa, synthetic construct
  VARIANT 1..30: note = This region may encompass 0-15 GS repeating units
GSGSGSGSGSGSGSGSGSGSGSGSGSGSGS

SEQ ID NO: 130 - protein, 30 aa, synthetic construct
  VARIANT 1..30: note = This region may encompass 0-15 SG repeating units
SGSGSGSGSGSGSGSGSGSGSGSGSGSGSG

SEQ ID NO: 131 - protein, 45 aa, synthetic construct
  VARIANT 1..45: note = This region may encompass 0-15 GSG repeating units
GSGGSGGSGGSGGSGGSGGSGGSGGSGGSGGSGGSGGSGGSGGSG

SEQ ID NO: 132 - protein, 60 aa, synthetic construct
  VARIANT 1..60: note = This region may encompass 0-15 SGSG repeating units
SGSGSGSGSGSGSGSGSGSGSGSGSGSGSGSGSGSGSGSGSGSGSGSGSGSGSGSGSGSG

SEQ ID NO: 133 - protein, 5 aa, synthetic construct
APVKQ

SEQ ID NO: 134 - protein, 791 aa, Synthetic construct
MADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVD

SEQ ID NO: 135 - protein, 791 aa, synthetic construct
MESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPHCYEQLEDSSEDEVDMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCC

SEQ ID NO: 136 - protein, 791 aa, Synthetic construct
MHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVD

SEQ ID NO: 137 - protein, 776 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 138 - protein, 762 aa, synthetic construct
MADDSALHKKYPFLNLLHTPPHRPPPLCPQAPRKTQCKRRLGNEHEESNSPLATPCTAEIYAYAYKNLKVVTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVD

SEQ ID NO: 139 - protein, 680 aa, synthetic construct
MHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPTAEIYAYAYKNLKVVQCTETDIREVQQLLLGTLNIVCPICAPKTTAEIYSYAYKQLKVLTAEIYAYAYKNLKVVMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVD

SEQ ID NO: 140 - protein, 806 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPTAEIYAYAYKNLKVV

SEQ ID NO: 141 - protein, 820 aa, synthetic construct
QCTETDIREVQQLLLGTLNIVCPICAPKTMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLPMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVD

SEQ ID NO: 142 - protein, 905 aa, synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTMADDSALHKKYPFLNLLHTPPHRPPPLCPQAPRKTQCKRRLGNEHEESNSPLATPCTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMHGRHVTLKDIVLDLQPPDPVGLHCYEQLVDSSEDEVDEVDGQDSQPLKQHFQIVTCCMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 143 - protein, 791 aa, Synthetic construct
MESISKRLDSCSEQLLELSEENSTDLHKHVLHWKCMRHSSVLLYKSKQMGLSHIGMQVVPPLKVSESKGHNSIEMQMHLESLLRTEYSMEPWTLQETSYSMWQTPPKRCFKKRGKTVEVKFDGCSNNTMDYVVWTDVYVQDNDTWVKVHSMVDSKGIYYTCGQFKTYYVNFVKESEKYGSTKHWEVCYGSTVICSPSSVSSTTQEVSIPESTTYTPSQTSTLVSSSTKEDSVQTPPRKRSRGVQQSPCNSLCVSHIGPVDSGNHNLITNNHDQHQRRNNSNSSSTPIVQFQGESNCLKCFRYRLNDRHRHLFDLISSTWHWSSSKSPHKHSIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 144 - protein, 791 aa, synthetic construct
MESISKRLDSCSEQLLELSEENSTDLHKHVLHWKCMRHSSVLLYKSKQMGLSHIGMQVVPPLKVSESKGHNSIEMQMHLESLLRTEYSMEPWTLQETSYSMWQTPPKRCFKKRGKTVEVKFDGCSNNTMDYVVWTDVYVQDNDTWVKVHSMVDSKGIYYTCGQFKTYYVNFVKESEKYGSTKHWEVCYGSTVICSPSSVSSTTQEVSIPESTTYTPSQTSTLVSSSTKEDSVQTPPRKRSRGVQQSPCNSLCVSHIGPVDSGNHNLITNNHDQHQRRNNSNSSSTPIVQFQGESNCLKCFRYRLNDRHRHLFDLISSTWHWSSSKSPHKHSIVTVTYDSEEQRQQFLDVVKIPPTISHKLGFMSLHLLHCYEQIEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 145 - protein, 794 aa, Synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCCYEQLEDSSEIDEVDGMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 146 - protein, 799 aa, Synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYVQTNHGLIAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 147 - protein, 789 aa, Synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYKNLKVVQCTETDIREVQQLLLGTLNIVCPICAPKTTAEIYSYAYKQLKVLQCKRRLGNEHEESNSPLATPCVWPTLDPWTVETTTSSLTITTSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 148 - protein, 787 aa, Synthetic construct
MEAIAKRLDACAEQLLELAEENSTDLHKHVLHWKCMRHASVLLYKAKQMGLSHIGMQVVPPLKVSEAKGHNAIEMQMHLESLLRTEYSMEPWTLQETSYAMWQTPPKRCFKKRGKTVEVKFDGCANNTMDYVVWTDVYVQDNDTWVKVHSMVDAKGIYYTCGQFKTYYVNFVKEAEKYGSTKHWEVCYGSTVICSPASVSSTTQEVSIPESTTYTPAQTSTLVSSSTKEDAVQTPPRKRARGVQQSPCNALCVAHIGPVDSGNHNLITNNHDQHQRRNNSNSSATPIVQFQGESNCLKCFRYRLNDRHRHLFDLISSTWHWASSKAPHKHAIVTVTYDSEEQRQQFLDVVKIPPTISHKLGFMSLHLLHCYEQLEDSSEDEVDMADDSALHKKYPFLNLLHTPPHRPPPLCPQAPRKTQCKRRLGNEHEESNSPLATPCTAEIYAYAYKNLKVVQCTETDIREVQQLLLGTLNIVCPICAPKTTAEIYSYAYKQLKVLQCKRRLNEHEESNSPLATPCVWPDPWTVETTTSSLTITSTKDGTTVTVQLRTAEIYAYAYKNLKVVMHGRHVTLKDIVLDLQPPDPVGLHCYEQLVDSSEDEVDEVDGQDSQPLKQHFQIVTCCHCYEQLEDSSEDEVDMESANASTSATTIDQLCKTFNLSMHTLQINCVFCKNALTTAEIYSYAYKHLKVLFRGGYPYAACACCLEFHGKINQYAHFDYAGYATTVEEETKQDILDVLIRCYLCHKPQCEVEKVKHILTKARFIKLNCTRKGRCLHCWTTCMEDMLP

SEQ ID NO: 149 - protein, 18 aa, synthetic construct
HCCYEQLEDSSEIDEVDG

SEQ ID NO: 150 - protein, 5 aa, synthetic construct
NLKVV

SEQ ID NO: 151 - protein, 23 aa, synthetic construct
TAEIYAYVQTNHGLIAYKNLKVV

SEQ ID NO: 152 - protein, 13 aa, synthetic construct
TAEIYAYKNLKVV

SEQ ID NO: 153 - protein, 51 aa, synthetic construct
QCKRRLNEHEESNSPLATPCVWPDPWTVETTTSSLTITTTKDGTTVTVQLR